(19) United States
(12) Patent Application Publication
(10) Pub. No.: US 2024/0398805 A1
(43) Pub. Date: Dec. 5, 2024

(54) QUINAZOLINE DERIVED COMPOUNDS AS EGFR INHIBITORS AND THEIR USES THEREOF

(71) Applicant: ACCUTAR BIOTECHNOLOGY INC., Brooklyn, NJ (US)

(72) Inventors: Yimin Qian, Plainsboro, NJ (US); Wei HE, Zionsville, IN (US); Robert LUO, New City, NY (US); Jie Fan, New York, NY (US)

(73) Assignee: ACCUTAR BIOTECHNOLOGY INC., Cranbury, NJ (US)

(21) Appl. No.: 18/263,993

(22) PCT Filed: Feb. 4, 2022
(86) PCT No.: PCT/US2022/015236

§ 371 Date: Aug. 2, 2023

Related U.S. Application Data
(60) Provisional application No. 63/146,208, filed on Feb. 5, 2021.

(51) Int. Cl. A61K 31/517; A61K 31/5377; A61K 31/541; A61K 31/553; A61P 35/00; C07D 401/14; C07D 405/14; C07D 413/14; C07D 417/14; C07D 471/04; C07D 487/04; C07D 491/056; C07D 491/107; C07D 498/08

(52) U.S. Cl. CPC A61K 31/517; A61K 31/5377; A61K 31/541; A61K 31/553; A61P 35/00; C07D 401/14; C07D 405/14; C07D 413/14; C07D 417/14; C07D 471/04; C07D 487/04; C07D 491/056; C07D 491/107; C07D 498/08

(57) ABSTRACT

The disclosure provides quinazoline based compounds and pharmaceutical compositions thereof, which are useful for inhibiting EGFR, as well as methods for using such compounds to treat cancer associated with an EGFR or HER2 exon 20 insertion mutation.

This application claims priority from U.S. Provisional Patent Application No. 63/146,208, filed Feb. 5, 2021, which is hereby incorporated by reference in its entirety.

### FIELD OF THE DISCLOSURE

The present invention relates to novel quinazoline derived compounds, or pharmaceutically acceptable salts thereof, which possess anti-tumor activity and are accordingly useful in methods of treatment of the human or animal body. In particular, the present invention relates to compounds that bind irreversibly to Epidermal Growth Factor Receptor (EGFR), pharmaceutical compositions comprising the compounds, and methods of use therefor.

### BACKGROUND OF THE DISCLOSURE

Protein kinases are a group of enzymes that regulate diverse, important biological processes including, for example, cell growth, proliferation, survival, invasion and differentiation, organ formation, tissue repair and regeneration. Protein kinases exert their physiological functions through catalyzing the phosphorylation of protein and thereby modulating cellular activities. Because protein kinases have profound effects on cells, their activities are highly regulated. Kinases are turned on or off by phosphorylation (sometimes by autophosphorylation), by binding of activator proteins or inhibitor proteins, or small molecules, or by controlling their location in the cell relative to their substrates.

EGFR is a transmembrane protein tyrosine kinase member of the erbB receptor family. Upon binding of a growth factor ligand such as epidermal growth factor (EGF), the receptor can homo-dimerize with another EGFR molecule or hetero-dimerize with another family member such as erbB2 (HER2), erbB3 (HER3), or erbB4 (HER4).

Homo- and/or hetero-dimerization of erbB receptors results in the phosphorylation of key tyrosine residues in the intracellular domain and leads to the stimulation of numerous intracellular signal transduction pathways involved in cell proliferation and survival.

Deregulation of erbB family signaling promotes proliferation, invasion, metastasis, angiogenesis, and tumor cell survival and has been described in many human cancers, including those of the lung, head and neck and breast.

The erbB family therefore represents a rational target for anticancer drug development and a number of agents targeting EGFR or erbB2 are now clinically available, including gefitinib (IRESSA®), erlotinib (TARCEVA®) and lapatinib (TYKERB®). Detailed reviews of erbB receptor signaling and its involvement in tumorigenesis are provided in New England Journal of Medicine (2008) Vol. 358, 1160-74 and Biochemical and Biophysical Research Communications (2004) Vol. 319, 1-11.

In 2004 it was reported (Science [2004] Vol. 304, 1497-500 and New England Journal of Medicine [2004] Vol. 350, 2129-39) that activating mutations in EGFR correlated with response to gefitinib therapy in non-small-cell lung cancer (NSCLC). The most common EGFR activating mutations, L858R and delE746_A750, result in an increase in affinity for small molecule tyrosine kinase inhibitors such as gefitinib and erlotinib and a decrease in affinity for adenosine triphosphate (ATP) relative to wild type (WT) EGFR. Ultimately, acquired resistance to therapy with gefitinib or erlotinib arises, for example by mutation of the gatekeeper residue T790M, which is reportedly detected in 50% of clinically resistant patients. This mutation is not believed to hinder the binding of gefitinib or erlotinib to EGFR sterically, merely to alter the affinity to ATP to levels comparable to WT EGFR.

In view of the importance of this mutation in resistance to existing therapies targeting EGFR, it is believed that agents which can inhibit EGFR harboring the gatekeeper mutation may be especially useful in the treatment of cancer.

Exon20 insertion mutations represents the third most common erbB family activating mutations in NSCLC. EGFR exon 20 insertion mutations are collectively representing approximately 4% to 10% of all EGFR mutations. Most of EGFR exon 20 insertion mutations occur near the end of αC-helix after residue Met766, with EGFR D770_N771 insSVD and V769_D770insASV accounting for about 40% of them. As EGFR exon20 insertion mutations, erbB2 exon20 insertion mutations occur in a similar prevalence in NSCLC and also in a similar position after residue Met774, with erbB2 A775_G776insYVMA accounting for about 80% of them. See, Jang, J. et al. Angew. Chem. Int. Ed. (2018) Vol. 57(36), 11629-11633.

HER2 mutations are reportedly present in about 2-4% of NSCLC (See, Stephens et al. Nature (2004) Vol. 431, 525-526). The most common mutation is an in-frame insertion within exon 20. In 83% of patients having HER2 associated NSCLC, a four amino acid YVMA insertion mutation occurs at codon 775 in exon 20 of HER2. (See, Arcila et al. Clin Cancer Res (2012) Vol. 18, 4910-4918). The exon 20 insertion results in increased HER2 kinase activity and enhanced signaling through downstream pathways, resulting in increased survival, invasiveness, and tumorigenicity (See, Wang et al. Cancer Cell (2006) Vol. 10, 25-38). Tumors harboring the HER2 YVMA mutation are largely resistant to known EGFR inhibitors. (See, Arcila et al. 2012).

Exon 20 insertion mutations are not restricted to lung cancer. Recent analysis of sinonasal squamous cell carcinoma (SNSCC), a rear form of head and neck cancer, demonstrated a remarkably high frequency of EGFR mutations (77% of SNSCC tumors), the majority of which were exon 20 insertions (88% of all EGFR mutations). See, Udager, A. M. et al. Cancer Res. (2015) Vol. 75, 2600-2606.

Exon 20 insertion mutations rarely respond to treatment with currently approved EGFR and HER2 TKIs, such as gefitinib, erlotinib or afatinib, or chemotherapies.

There remains a need for compounds that may exhibit favorable potency profiles against WT EGFR versus activating mutant forms of EGFR/erbB2 (for example the L858R EGFR mutant, or the delE746_A750 mutant or the Exon19 deletion EGFR mutant, or EGFR/erbB2 exon20 insertion mutations) and/or resistant mutant forms of EGFR (for example T790M EGFR mutant), and/or selectivity over other enzyme receptors which may make the compounds especially promising for development as therapeutic agents. In this regard, there remains a need for compounds that show a higher inhibition of certain activating or resistance mutant forms of EGFR and HER2 while at the same time showing relatively low inhibition of WT EGFR. Such compounds may be expected to be more suitable as therapeutic agents, particularly for the treatment of cancer, due to reduction of toxicology associated with WT EGFR inhibition. Such toxicologies are known to manifest themselves in man as skin rashes and/or diarrhea. The inventors have found novel quinazoline derived compounds that have high potency against several mutant forms of EGFR and HER2 while at the same showing relatively low inhibition of WT EGFR.

### SUMMARY OF THE DISCLOSURE

The invention provides compounds, compositions and methods for modulating the activity of EGFR.

In one aspect, the invention provides compounds which act as inhibitors of EGFR.

In some embodiments, the present disclosure is directed to a compound of Formula (I), or a tautomer, stereoisomer or a mixture of stereoisomers, or a pharmaceutically acceptable salt, or hydrate, or deuterated derivative thereof:

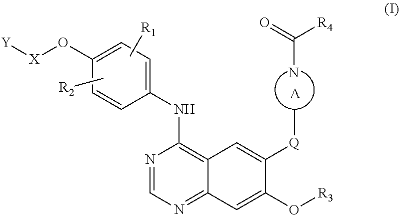

wherein:
X is absent or is chosen from 5- to 6-member cycloalkyl, 5- to 6-member aryl, 5- to 6-member heterocycle, or a monocyclic or bicyclic 5- to 12-member heteroaryl, each of which is independently substituted with 0, 1, 2, or 3 R^{a};
Y is absent or is chosen from an ester group, a carboxylic acid group, an amide group, an group, an alkylamino group, a dialkylamino group, C₁₋₄ alkyl, O-heterocycle, NH-heterocycle, 5- to 6-member cycloalkyl, 5- to 6-member aryl, 4- to 10-member monocyclic or bicyclic heterocycle, or a monocyclic or bicyclic 5- to 12-member heteroaryl, each of which is independently substituted with 0, 1, 2, or 3 R^{a};
R₁ and R₂ are each independently chosen from hydrogen, halogen, —CN, hydroxyl, C₁-C₅alkyl, deuterated C₁-C₅alkyl, C₁-C₅alkoxy, deuterated C₁-C₅alkoxy, and C₁-C₅haloalkyl;
R₃ is chosen from H, C₁-C₅alkyl, deuterated C₁-C₅alkyl, C₁-C₅alkoxy, C₁-C₅haloalkyl, C₃-C₇cycloalkyl, and heterocycle, wherein each is independently substituted with 0, 1, 2, or 3 R^{a};
R₄ is C₂₋₆ alkenyl or C₂₋₆ alkynyl, wherein each is independently substituted with 0, 1, 2, or 3 R^{a};
Q is “NH” or “O”;
Ring A is chosen from piperidine, pyrrolidine, bicyclic amine, bridge cyclic amine and a amine, each of which is optionally substituted with halogen, C₁-C₅alkyl, or C₁-C₅alkoxy; and
R^{a} is independently chosen from hydrogen, deuterium, hydroxyl, halogen, amino group, alkylamino group, dialkylamino group, —CN, C₁₋₆ alkyl, deuterated C₁₋₆ alkyl, C₁₋₆ alkoxyl, deuterated C₁₋₆ alkoxyl, C₁₋₆ haloalkyl, C₁₋₆ haloalkoxyl, C₃₋₆ cycloalkyl, heterocycle, heterocycloalkyl, aryl, and heteroaryl.
   In some embodiments, X is chosen from

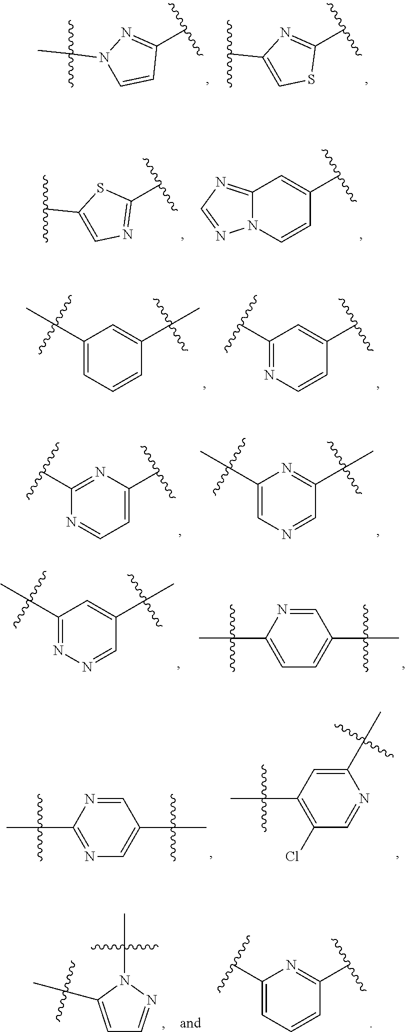

In some embodiments, Y is chosen from

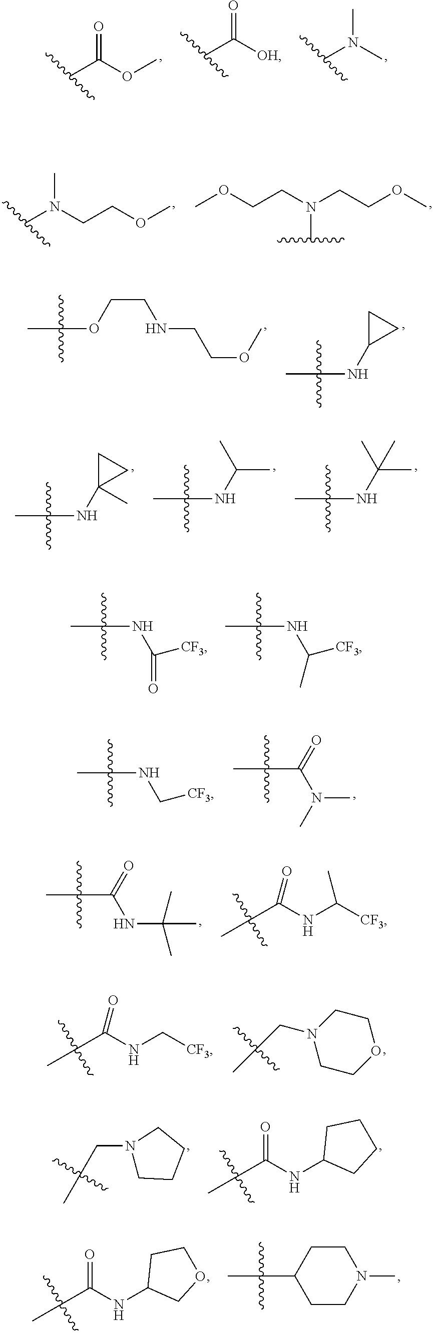

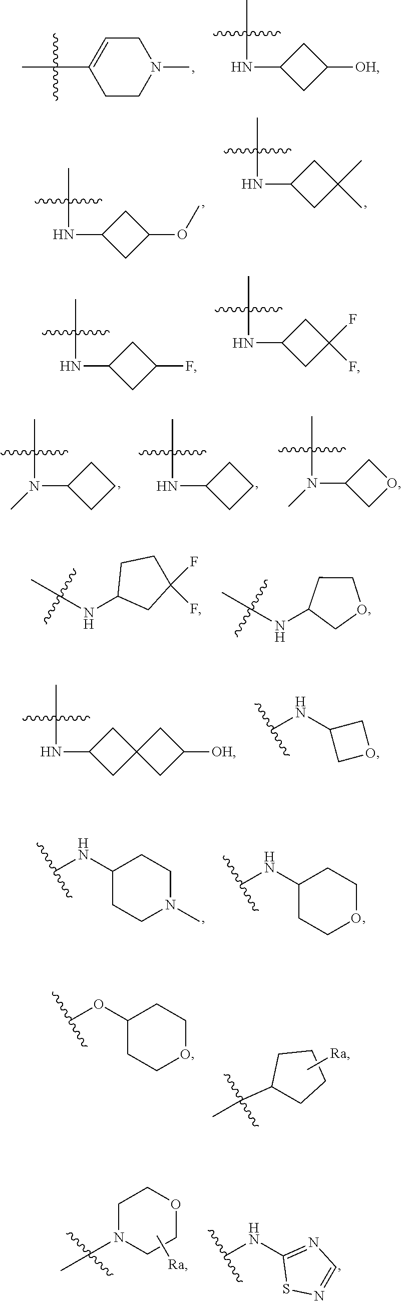

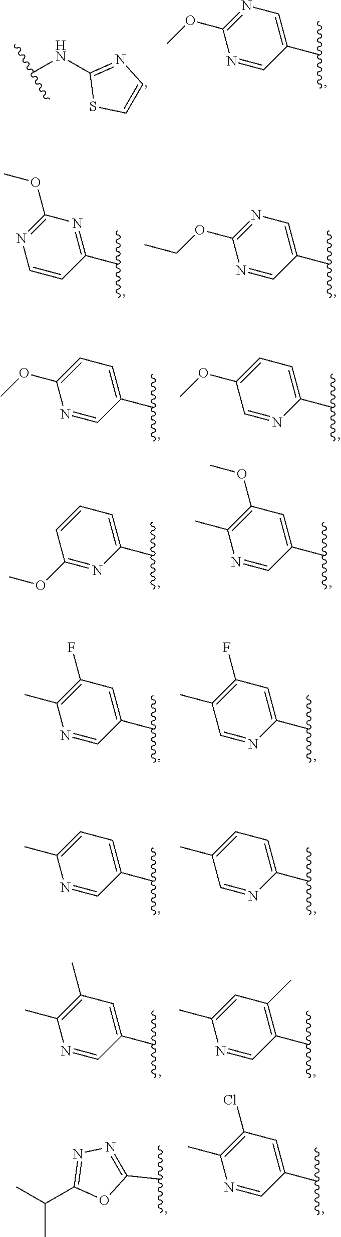

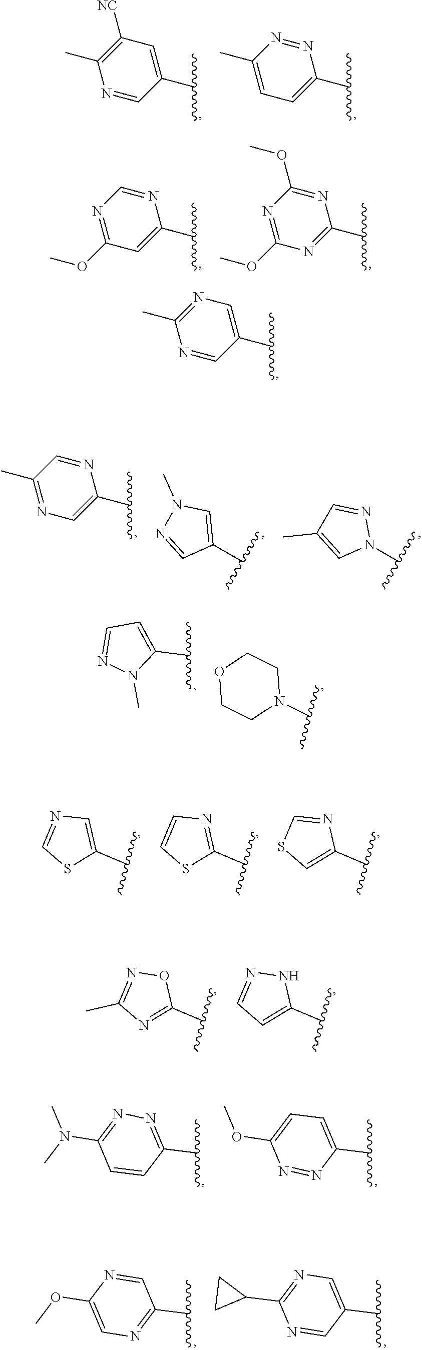

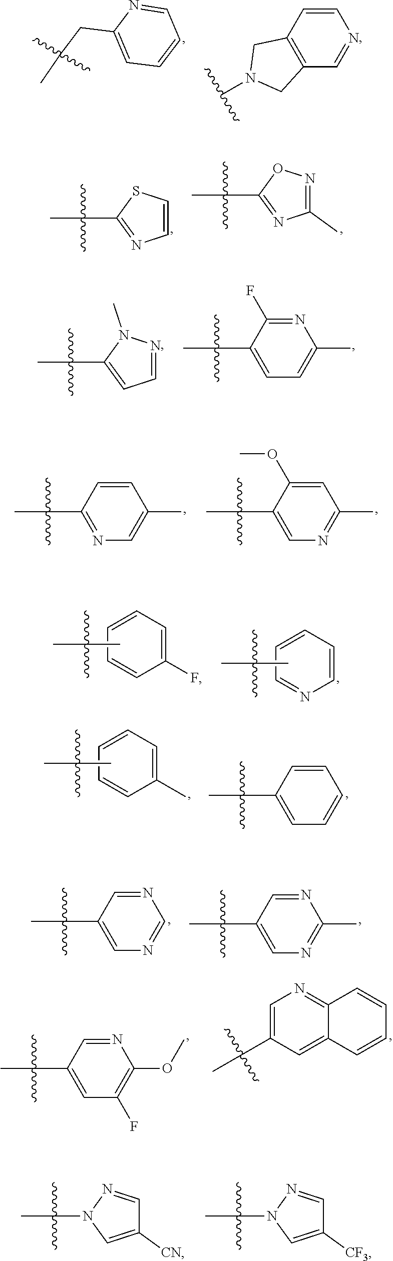

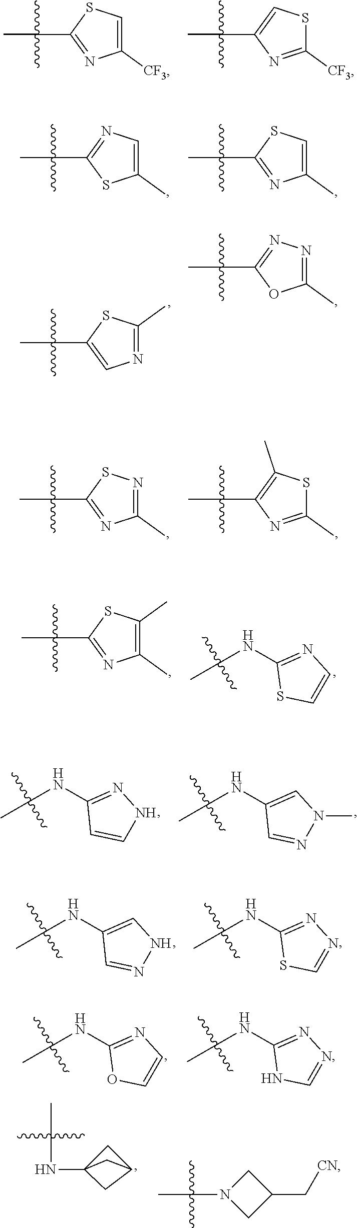

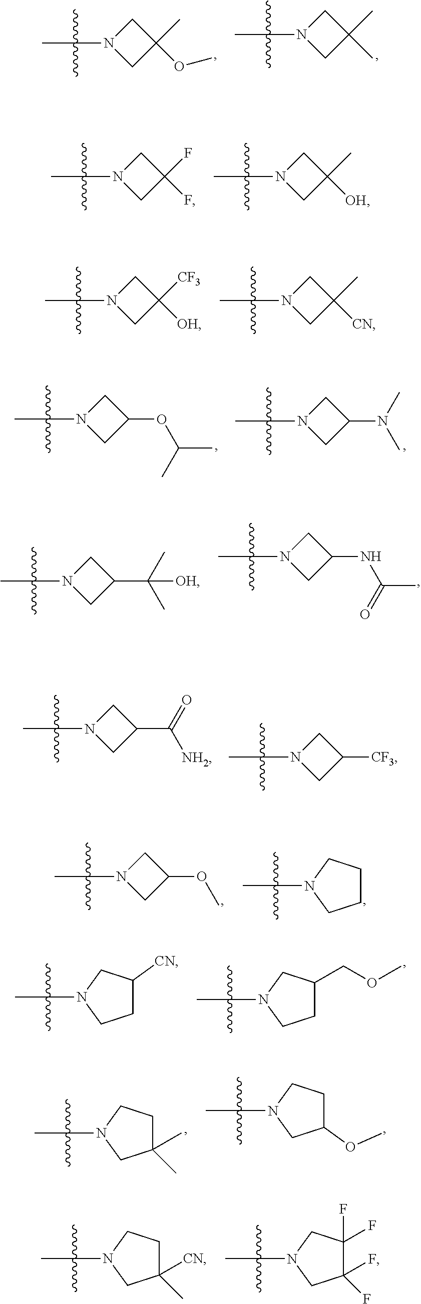

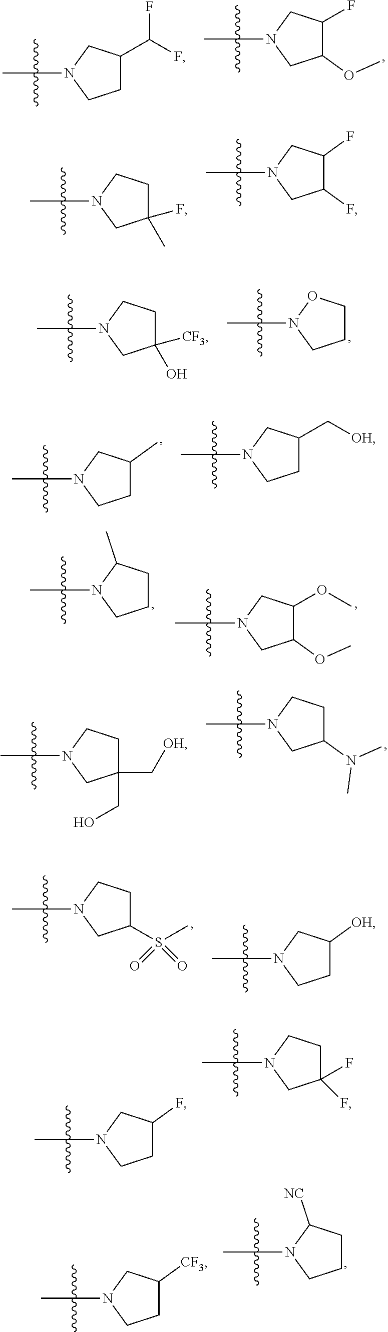

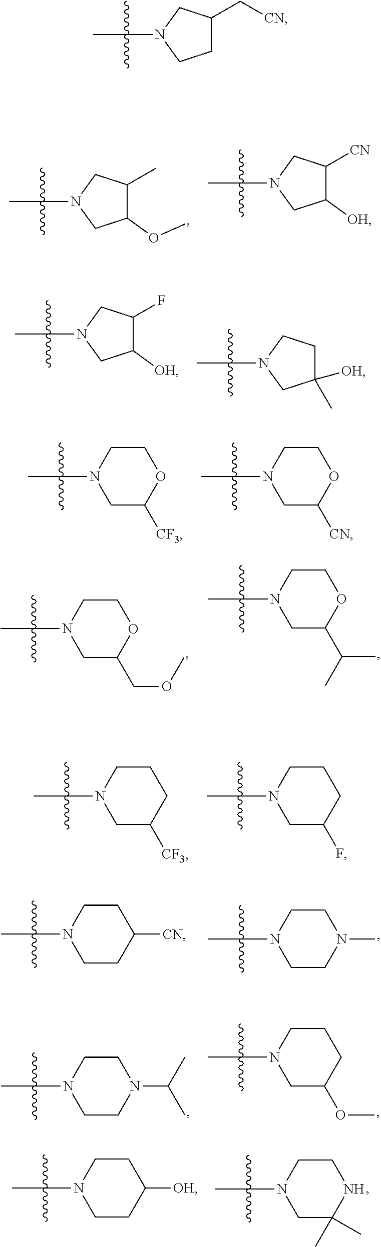

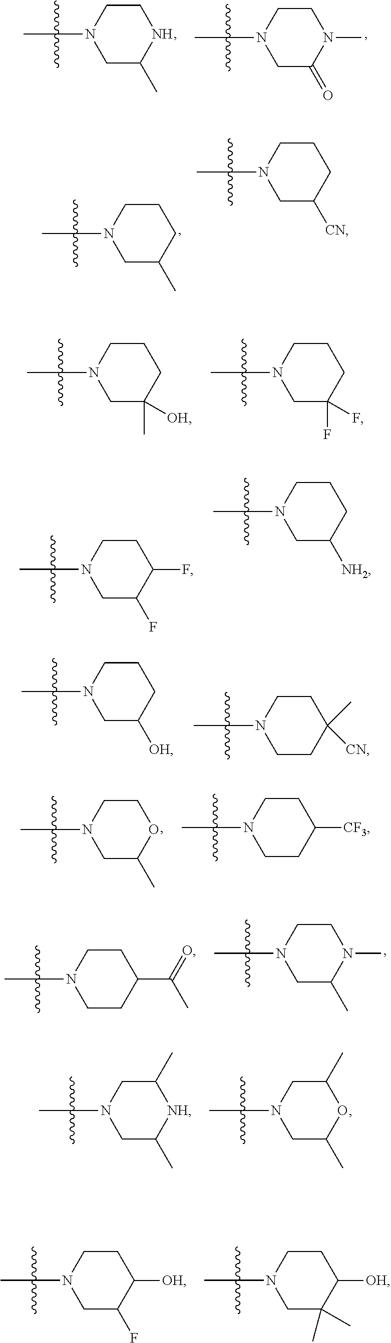

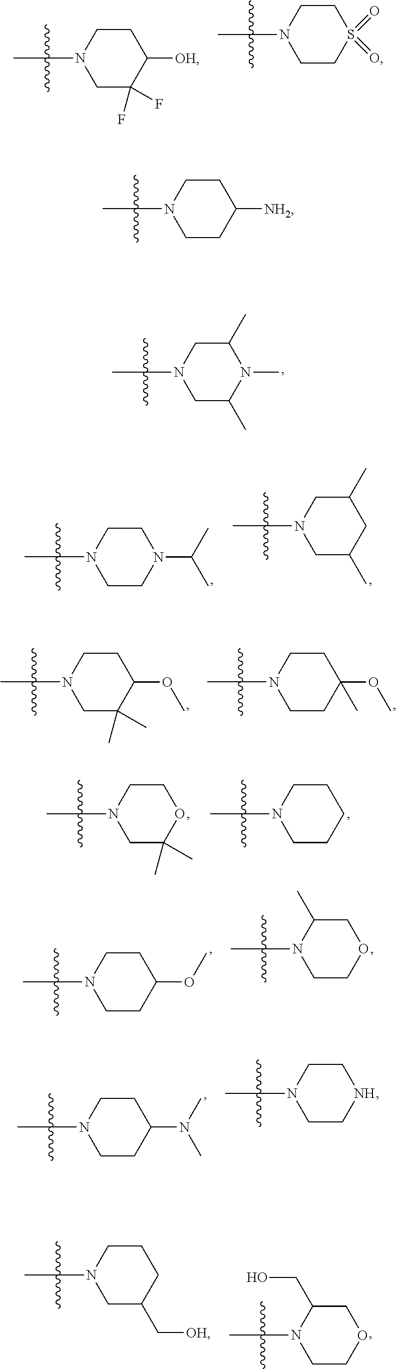

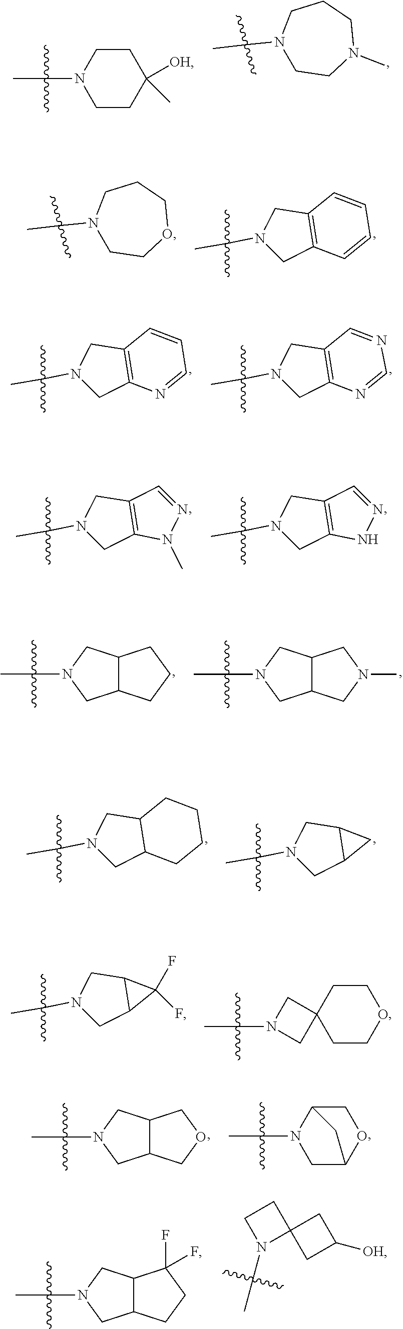

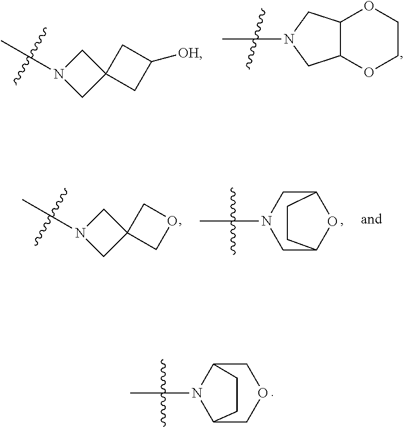 [SMILES: *N1CC2(CC(O)C2)C1 ; *N1CC2OCCOC2C1 ; *N1CC2(COC2)C1 ; *N1CC2CCC(C1)O2 ; *N1C2CCC1COC2]

In some embodiments, R₁ and R₂ are each independently chosen from hydrogen, halogen, —ON, C₁₋₄alkyl, and C₁₋₄haloalkyl.

In some embodiments, R₃ is a C₁₋₄ alkyl. In some embodiments, R₃ is chosen from CD₃, —CH₃, —CH₂—CH₃, —CH(CH₃)₂, —CH₂CH₂OCH₃, —CHF₂, —CH₂CHF₂, cyclopropyl, —CH₂CH₂OH, CH₂CH₂N(CH₃)₂, CH₂CH₂NHCH₃,

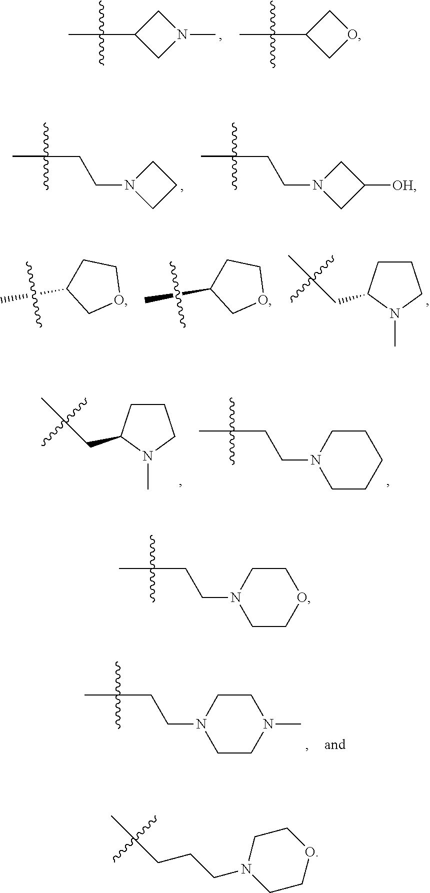

In some embodiments, R₄ is chosen from —CH═CH₂,

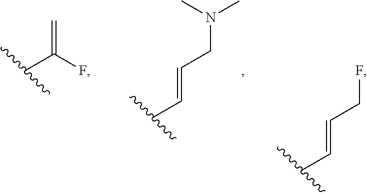

—C≡C—CH₃, —CH═CH—CH₃, and

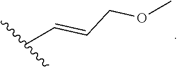 [SMILES: */C=C/COC]

In some embodiments, ring A is chosen from

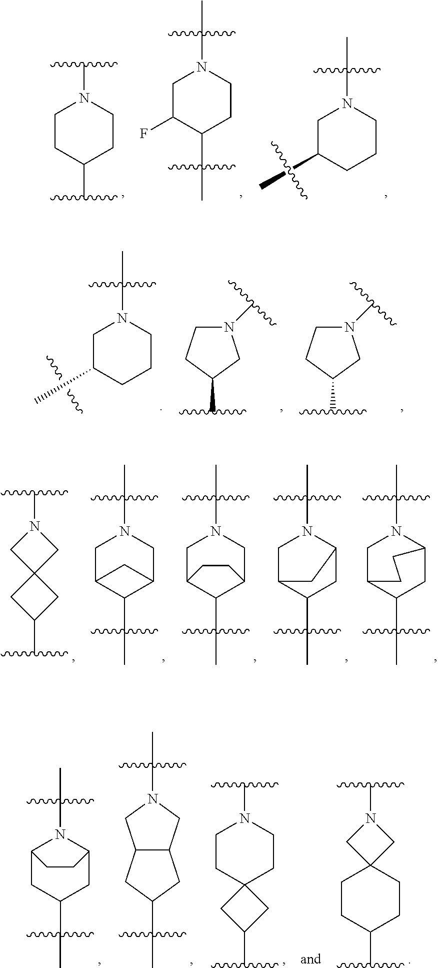

In some embodiments, the compound of Formula (I) may encompass both stereoisomers and a mixture of stereoisomers. In some embodiments, the compound of Formula (I) may encompass both racemic isomers and enantiomeric isomers.

In some embodiments, provided herein is a pharmaceutical composition comprising a compound of Formula (I), or a tautomer, stereoisomer or a mixture of stereoisomers, or a pharmaceutically acceptable salt, or hydrate, or deuterated derivative thereof, and one or more of a pharmaceutically acceptable carrier, a pharmaceutically acceptable vehicle, a pharmaceutically acceptable excipient, or combinations thereof.

Also disclosed herein is a combination therapy comprising a compound of Formula (I), or a tautomer, stereoisomer or a mixture of stereoisomers, or a pharmaceutically acceptable salt, or hydrate, or deuterated derivative thereof, and an additional agent.

In some embodiments, provided herein is the use of a compound of Formula (I), or a tautomer, stereoisomer or a mixture of stereoisomers, or a pharmaceutically acceptable salt, or hydrate, or deuterated derivative thereof, for inhibiting EGFR.

In some embodiments, provided herein is the use of a compound of Formula (I), or a tautomer, stereoisomer or a mixture of stereoisomers, or a pharmaceutically acceptable salt, or hydrate, or deuterated derivative thereof, in the manufacture of a medicament for treating a condition associated with EGFR or HER2 exon 20 insertion mutation.

In some embodiments, provided herein is the use of a compound of Formula (I), or a tautomer, stereoisomer or a mixture of stereoisomers, or a pharmaceutically acceptable salt, or hydrate, or deuterated derivative thereof, for treating a condition associated with EGFR or HER2 exon 20 insertion mutation.

In some embodiments, provided herein is the use of a compound of Formula (I), or a tautomer, stereoisomer or a mixture of stereoisomers, or a pharmaceutically acceptable salt, or hydrate, or deuterated derivative thereof, for treating cancer associated with EGFR or HER2 exon 20 insertion mutation, wherein the cancer is selected from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

In some embodiments, provided herein is a method for inhibiting EGFR, comprising administering to a system or subject a therapeutically effective amount of a compound of Formula (I), or a tautomer, stereoisomer or a mixture of stereoisomers, or a pharmaceutically acceptable salt, or hydrate, or deuterated derivative thereof.

Also disclosed herein is a method for treating a condition associated with EGFR or HER2 exon 20 insertion mutation, comprising administering to a system or subject in need of such treatment an effective amount of a compound of Formula (I), or a tautomer, stereoisomer or a mixture of stereoisomers, or a pharmaceutically acceptable salt, or hydrate, or deuterated derivative thereof.

### DETAILED DESCRIPTION OF THE DISCLOSURE

### Definitions

A dash (“-”) that is not between two letters or symbols is used to indicate a point of attachment for a substituent. For example, —CN is attached through the carbon atom.

When a range of values is listed, it is intended to encompass each value and sub-range within the range. For example, “C₁-C₆ alkyl” or “C₁₋₆alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C_{4- 6}, C₄₋₅, and C₅₋₆ alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C₁₋₁₀ acyl radical which refers to the total number of chain or ring atoms of the, for example, alkyl, alkenyl, alkynyl, aryl, cycloalkyl, or heteroaryl, portion plus the carbonyl carbon of acyl. For example, a C₄-acyl has three other ring or chain atoms plus carbonyl.

The term “alkenyl” as used herein refers to an unsaturated straight or branched hydrocarbon having at least one carbon-carbon double bond, such as a straight or branched group of 2 to 8 carbon atoms, referred to herein as C₂₋₈alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl, 2-butenyl, and 4-(2-methyl-3-butene)-pentenyl.

The term “alkyl” as used herein refers to a saturated straight or branched hydrocarbon, such as a straight or branched group of 1 to 8 carbon atoms, referred to herein as C1-8alkyl. Exemplary alkyl groups include, but are not limited to, methyl, ethyl, propyl, isopropyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-1-pentyl, 3 methyl-1-pentyl, 4-methyl-1-pentyl, 2-methyl-2-pentyl, 3-methyl-2-pentyl, 4 methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein refers to a divalent alkyl radical.

Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2 to 8 carbon atoms, referred to herein as C₂₋₈alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “aryl” as used herein refers to a mono-, bi-, or other multi carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “C₆-aryl.”

The term “cyano” as used herein refers to CN.

The term “cycloalkyl” as used herein refers to a saturated or unsaturated cyclic, bicyclic, or bridged bicyclic hydrocarbon group of 3-16 carbons, or 3-8 carbons, referred to herein as “C₃₋₈cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

The term “heteroatoms,” as used herein, refers to nitrogen (N), oxygen (O), sulfur (S) or phosphorus (P) atoms, wherein the N, S and P can optionally be oxidized to various oxidation states.

The term “heteroaryl” as used herein refers to a mono-, bi-, or multi-cyclic, aromatic ring system containing one or more heteroatoms, for example 1 to 3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Exemplary heteroaryl groups include, but are not limited to, a monocyclic aromatic ring, wherein the ring comprises 2 to 5 carbon atoms and 1 to 3 heteroatoms, referred to herein as “C₂₋₅heteroaryl.” Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups also include, but are not limited to, a bicyclic aromatic ring, wherein the ring comprises 5 to 14 carbon atoms and 1 to 3 heteroatoms, referred to herein as “C₅₋₁₄heteroaryl.” Representative examples of heteroaryl include, but not limited to, indazolyl, indolyl, azaindolyl, indolinyl, benzotriazolyl, benzoxadiazolyl, imidazolyl, cinnolinyl, imidazopyridyl, pyrazolopyridyl, pyrrolopyridyl, quinolinyl, isoquinolinyl, quinazolinyl, quinazolinonyl, indolinonyl, isoindolinonyl, tetrahydronaphthyl, tetrahydroquinolinyl, tetrahydroisoquinolinyl, and the like.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

“Spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. A (C₃₋₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The terms “composition” or “pharmaceutical composition,” as used herein, refers to a mixture of at least one compound, such as a compound Formula (I), or a tautomer, stereoisomer or a mixture of stereoisomers, or a pharmaceutically acceptable salt, or hydrate, or deuterated derivative thereof, with at least one and optionally more than one other pharmaceutically acceptable chemical components, such as carriers, stabilizers, diluents, dispersing agents, suspending agents, thickening agents, and/or excipients.

The terms “effective amount” or “therapeutically effective amount,” as used herein, refer to a sufficient amount of a compound described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “pharmaceutically acceptable,” as used herein, refers to a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “carrier,” as used herein, refers to chemical compounds or agents that facilitate the incorporation of a compound described herein into cells or tissues. The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329; Remington: The Science and Practice of Pharmacy, 21^{st} Ed. Pharmaceutical Press 2011; and subsequent versions thereof). Except insofar as any conventional carrier is incompatible with the active ingredient, its use in the therapeutic or pharmaceutical compositions is contemplated.

The term “pharmaceutically acceptable carrier” or “pharmaceutically acceptable vehicle” is used interchangeably and as is known to those skilled in the art, can be any and all solvents, dispersion media, Coatings, surfactants, antioxidants, preservatives (e.g., antimicrobial agents, antifungal agents), isotonic agents, absorption delaying agents, salts, preservatives, drug stabilizers, binders, excipients, disintegrants, lubricant, including sweeteners, flavors, dyes, and the like, and combinations thereof (e.g., Remington's Pharmaceutical Sciences, 18th Ed. Mack Printing Company, 1990, pp. 1289-1329). Except insofar as any conventional carrier is incompatible with the active ingredient, its use in therapeutic or pharmaceutical compositions is contemplated.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, “cancer” refers to diseases, disorders, and conditions that involve abnormal cell growth with the potential to invade or spread to other parts of the body.

Exemplary cancers include, but are not limited to, breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, a subject is “in need of” a treatment if such subject would benefit biologically, medically or in quality of life from such treatment.

As used herein, “exon 20 insertion mutation” refers to a mutation in which one or more amino acids (preferably 1 to 7, more preferably 1 to 4) are inserted in the exon 20 region of EGFR, and includes, but is not limited to, a mutation in which amino acid sequence SVD (serine, valine, and aspartic acid in this order from the N-terminus) is inserted between the 770th aspartic acid and 771st asparagine in the exon 20 region (D770_N771 insSVD); a mutation in which amino acid sequence ASV (alanine, serine, and valine in this order from the N-terminus) is inserted between the 769th alanine and 770th aspartic acid in the exon region (A769_D770insASV); and a mutation in which amino acid sequence YVMA (tyrosine, valine, methionine, and alanine in this order from the N-terminus) is inserted between the 775th alanine and 776th glycine in the exon region (A775_G776ins YVMA).

The term “administration” or “administering” of the subject compound means providing a compound of the invention, a pharmaceutically acceptable salt, a pharmaceutically acceptable solvate, or solvate thereof to a subject in need of treatment.

The term “combination” as used herein means a product that results from the mixing or combining of more than one active ingredient and includes both fixed and non-fixed combinations of the active ingredients. The term “fixed combination” means that the active ingredients, by way of example, a compound of Formula (I), or a pharmaceutically acceptable salt thereof, and an additional anti-cancer agent, are both administered to a patient simultaneously in the form of a single entity or dosage. The term “non-fixed combination” means that the active ingredients, by way of example, a compound of Formula (I) or a pharmaceutically acceptable salt thereof, and an additional anti-cancer agent, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, C, ¹³C, ¹⁴C, ¹⁵N, ¹⁸F, ³¹P, ³²P, ³⁵S, ³⁶Cl and ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. Isotopically-labeled compounds of the invention can generally be prepared by conventional techniques known to those skilled in the art or by processes analogous to those described in the accompanying Examples and Processes using an appropriate isotopically-labeled reagents in place of the non-labeled reagent previously employed.

### Compounds

In some embodiments, provided herein are compounds of Formula (I) or a tautomer, stereoisomer or a mixture of stereoisomers, or a pharmaceutically acceptable salt, or hydrate, or deuterated derivative thereof:

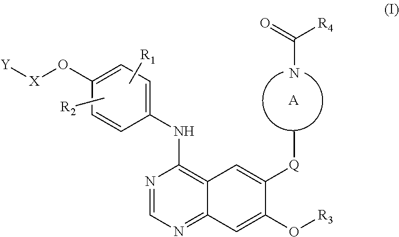

wherein:
X is absent or is chosen from 5- to 6-member cycloalkyl, 5- to 6-member aryl, 5- to 6-member heterocycle, or a monocyclic or bicyclic 5- to 12-member heteroaryl, each of which is independently substituted with 0, 1, 2, or 3 R^{a};
Y is absent or is chosen from an ester group, a carboxylic acid group, an amide group, an amino group, an alkylamino group, a dialkylamino group, C₁₋₄ alkyl, O-heterocycle, N-heterocycle, 5- to 6-member cycloalkyl, 5- to 6-member aryl, 4- to 10-member monocyclic or bicyclic heterocycle, or a monocyclic or bicyclic 5- to 12-member heteroaryl, each of which is independently substituted with 0, 1, 2, or 3 R^{a};
R₁ and R₂ are each independently chosen from hydrogen, halogen, —CN, hydroxyl, C₁-C₅alkyl, deuterated C₁-C₅alkyl, C₁-C₅alkoxy, deuterated C₁-C₅alkoxy, and C₁-C₅haloalkyl;
R₃ is chosen from H, C₁-C₅alkyl, deuterated C₁-C₅alkyl, C₁-C₅alkoxy, C₁-C₅haloalkyl, C₃-C₇cycloalkyl, and heterocycle, wherein each is independently substituted with 0, 1, 2, or 3 R^{a};
R₄ is C₂₋₆ alkenyl or C₂₋₆ alkynyl, wherein each is independently substituted with 0, 1, 2, or 3 R^{a};
Q is “NH” or “O”;
Ring A is chosen from piperidine, pyrrolidine, bicyclic amine, bridge cyclic amine and a spirocyclic amine, each of which is optionally substituted with halogen, C₁-C₅alkyl, or C₁-C₅alkoxy; and
R^{a} is independently chosen from hydrogen, deuterium, hydroxyl, halogen, amino group, alkylamino group, dialkylamino group, —CN, C₁₋₆ alkyl, deuterated C₁₋₆ alkyl, C₁₋₆ alkoxyl, deuterated C₁₋₆ alkoxyl, C₁₋₆ haloalkyl, C₁₋₆haloalkoxyl, C₃₋₆cycloalkyl, heterocycle, heterocycloalkyl, aryl, and heteroaryl.

In some embodiments, X is chosen from

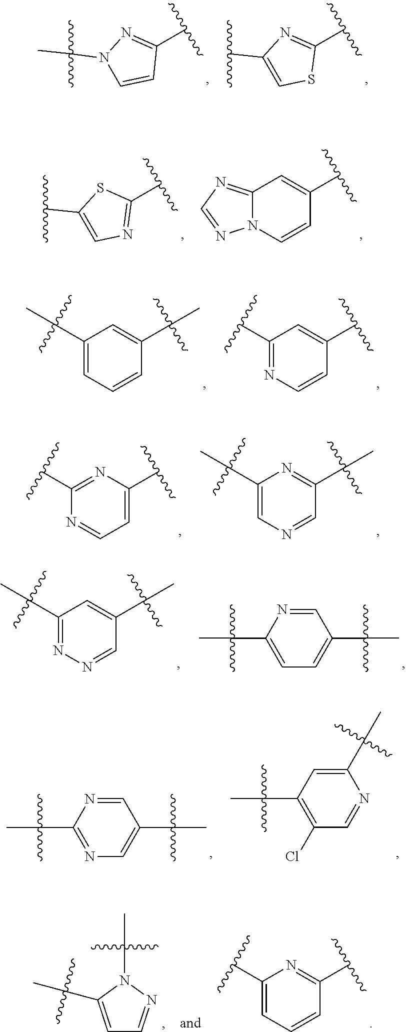

In some embodiments, Y is absent. In some embodiments, Y is chosen from

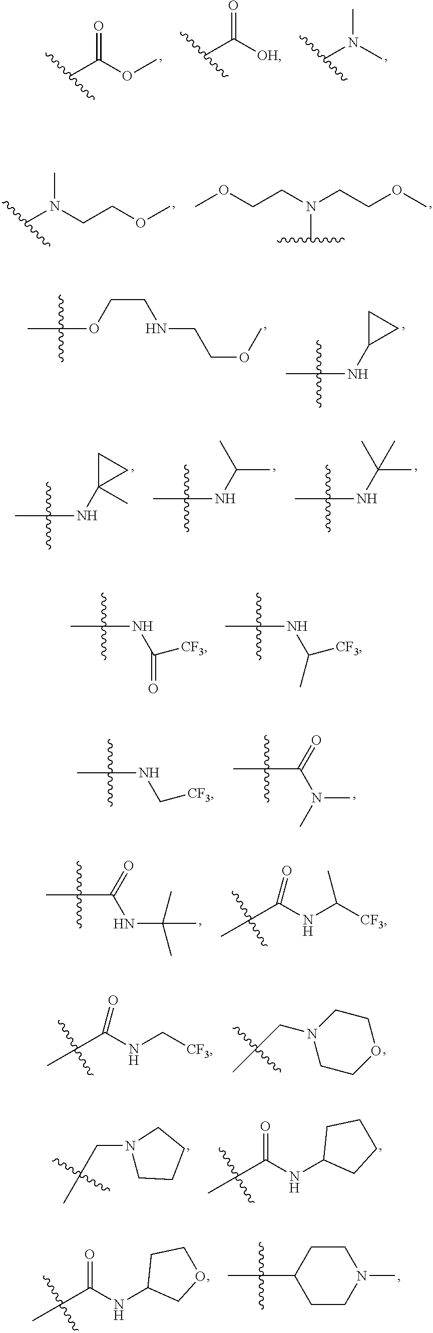

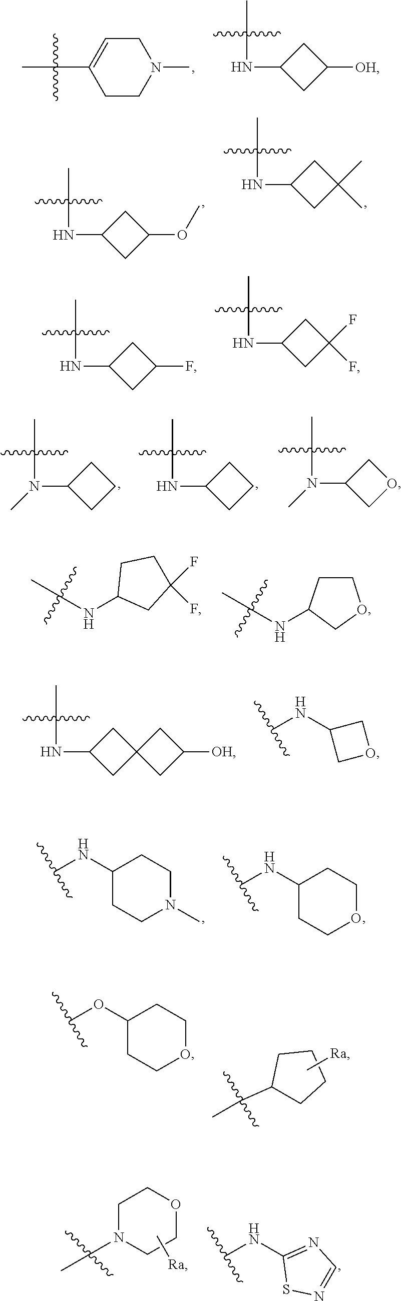

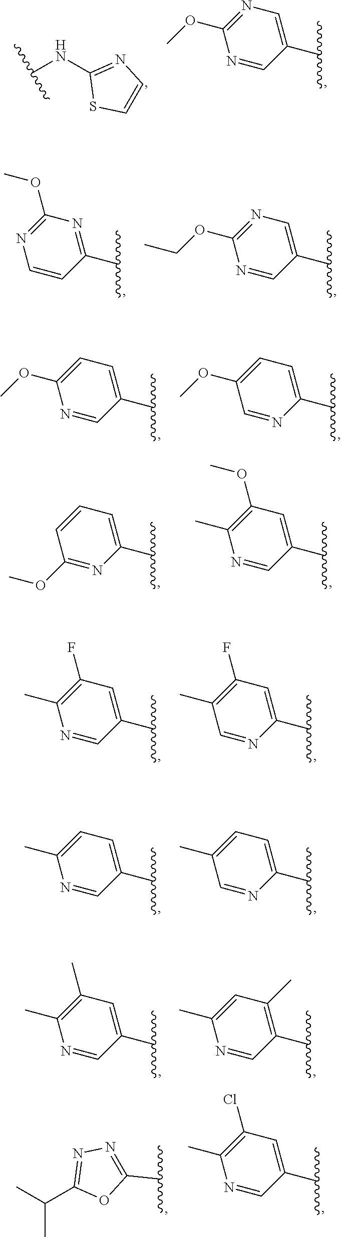

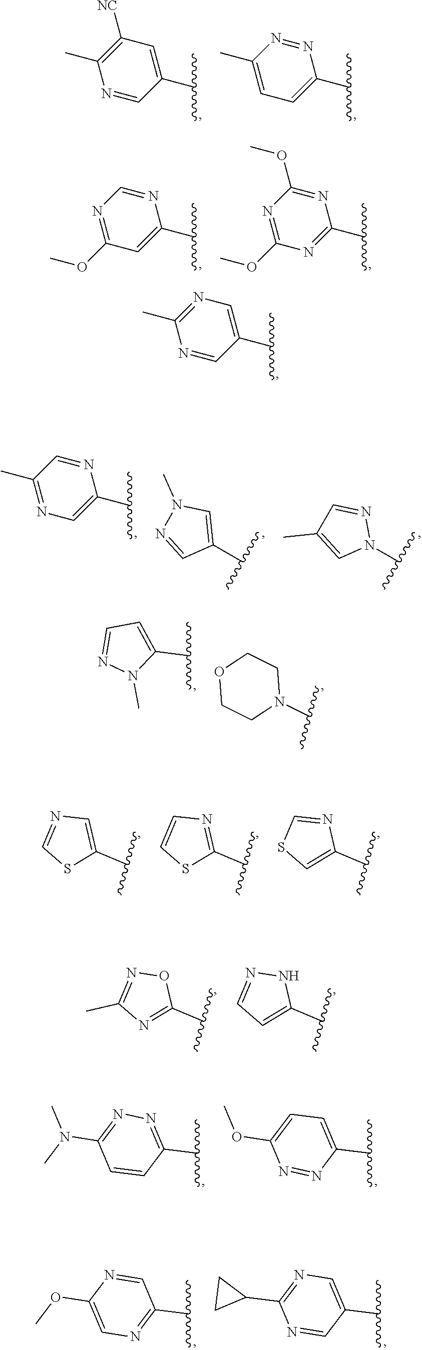

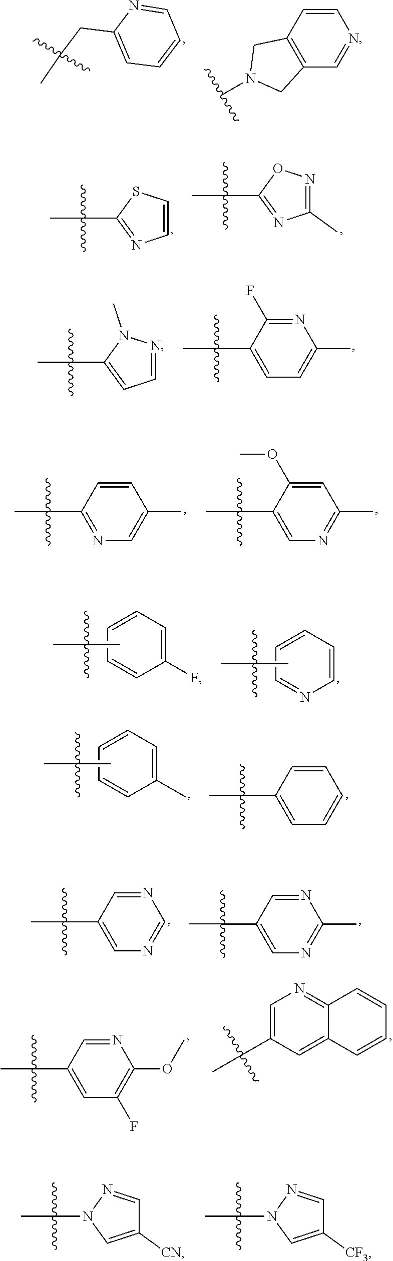

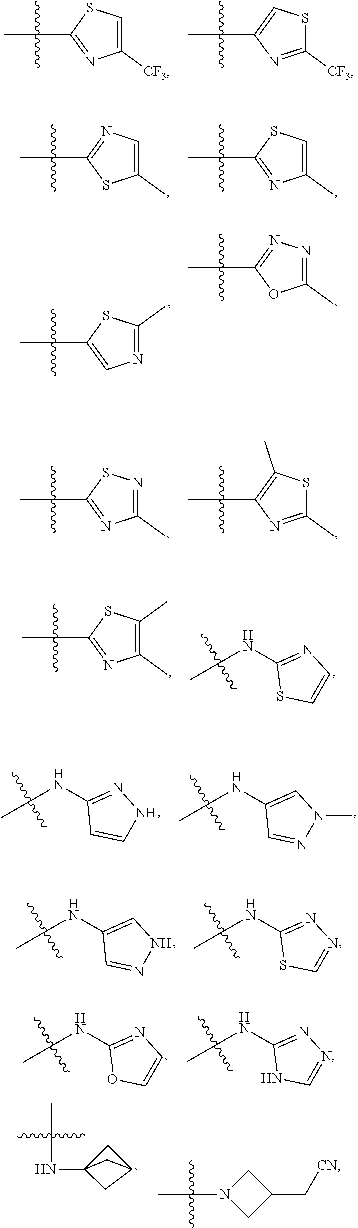

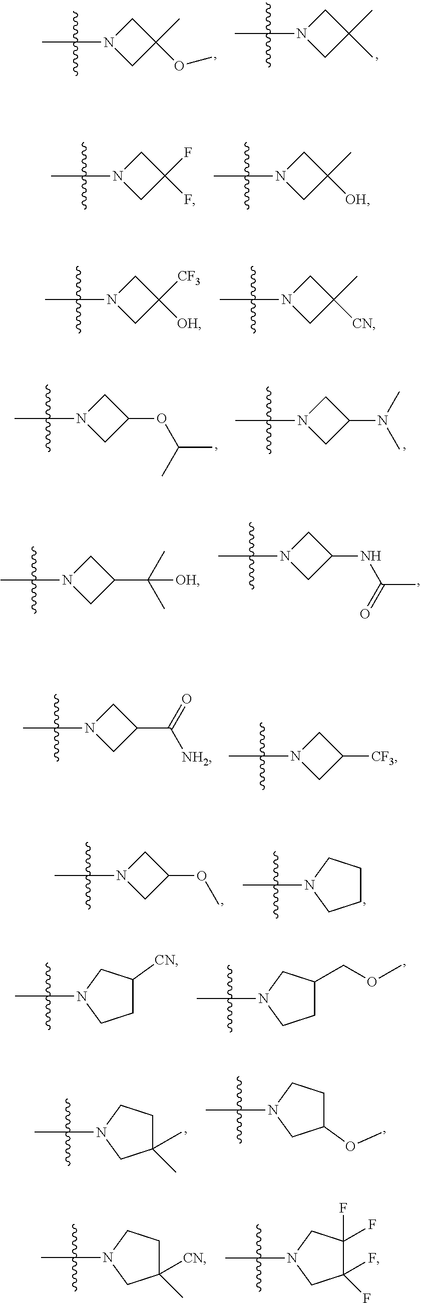

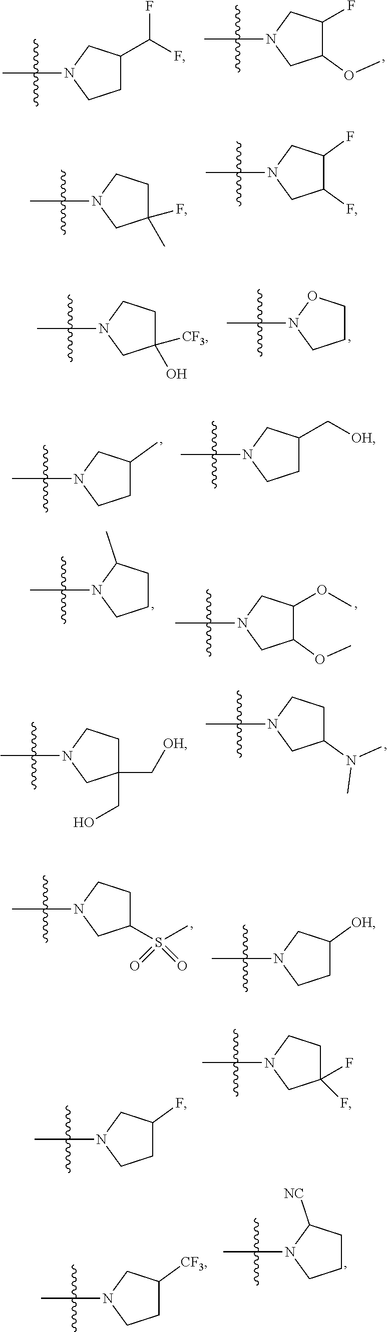

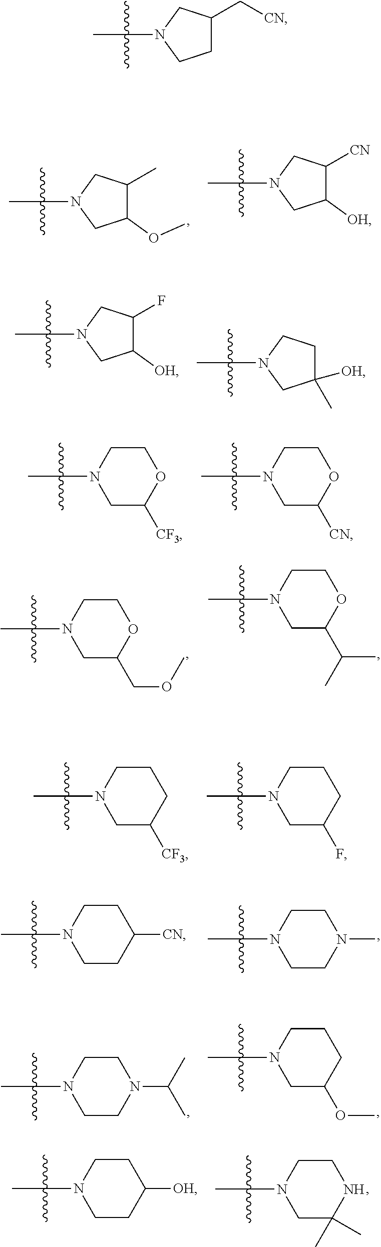

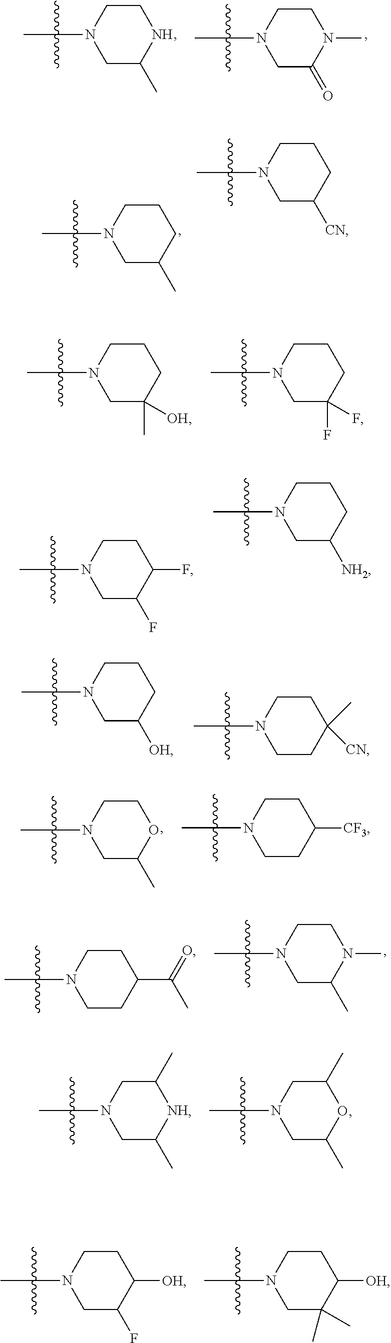

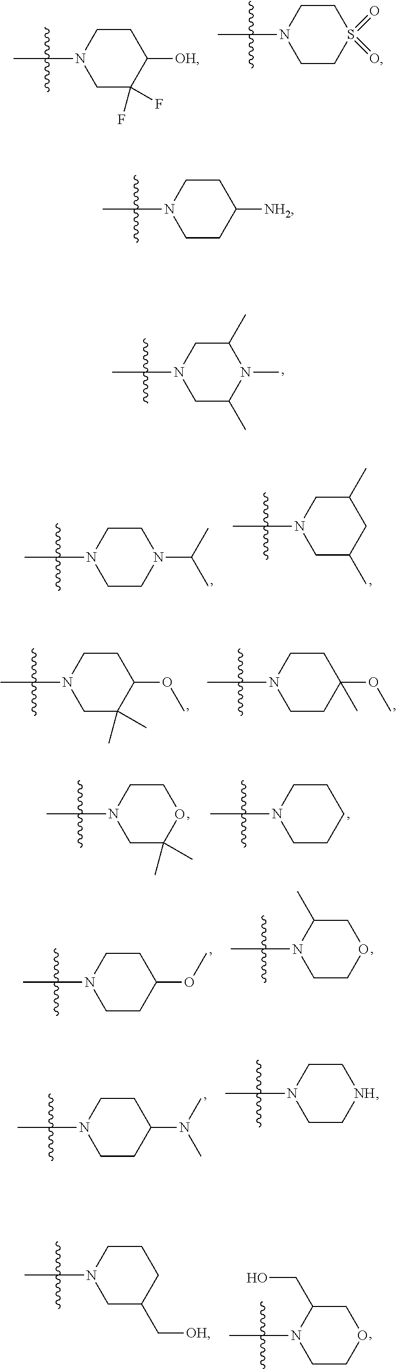

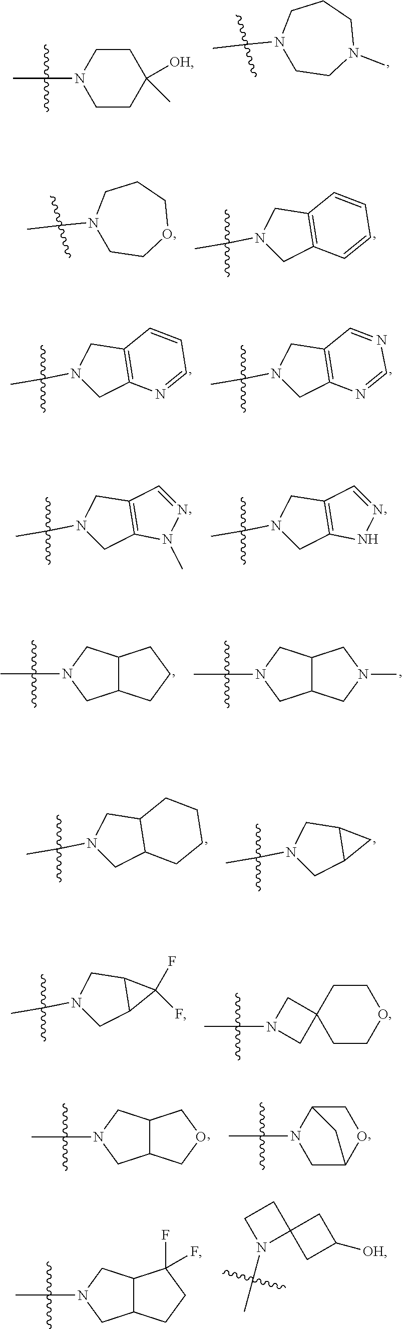

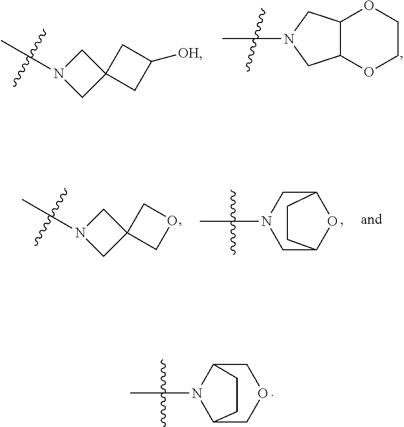 [SMILES: *N1CC2(CC(O)C2)C1 ; *N1CC2OCCOC2C1 ; *N1CC2(COC2)C1 ; *N1CC2CCC(C1)O2 ; *N1C2CCC1COC2]

In some embodiments, R₁ and R₂ are each independently chosen from hydrogen, halogen, —CN, C₁₋₄alkyl, and C₁₋₄haloalkyl. In another embodiment, R₁ and R₂ are each H. In one embodiment, R₁ is H and R₂ is F. In one embodiment, R₁ is F and R₂ is H. In one embodiment, R₁ is F and R₂ is Cl. In one embodiment, R₁ is Cl and R₂ is F. In one embodiment, R₁ is Cl and R₂ is H. In one embodiment, R₁ is H and R₂ is Cl.

In some embodiments, R₃ is a H, C₁₋₄ alkyl. In some embodiments, the C₁₋₄ alkyl is substituted with deuterium, halogen, hydroxyl, C₁₋₄ alkoxyl, amino group, alkylamino group, dialkylamino group, morpholinyl, piperidinyl, pyrrolidinyl, and azetidinyl. In another embodiment, R₃ is chosen from CD₃, —CH₃, —CH₂—CH₃, —CH(CH₃)₂, —CH₂CH₂OCH₃, —CHF₂, —CH₂CHF₂, cyclopropyl, —CH₂CH₂OH, CH₂CH₂N(CH₃)₂, CH₂CH₂NHCH₃,

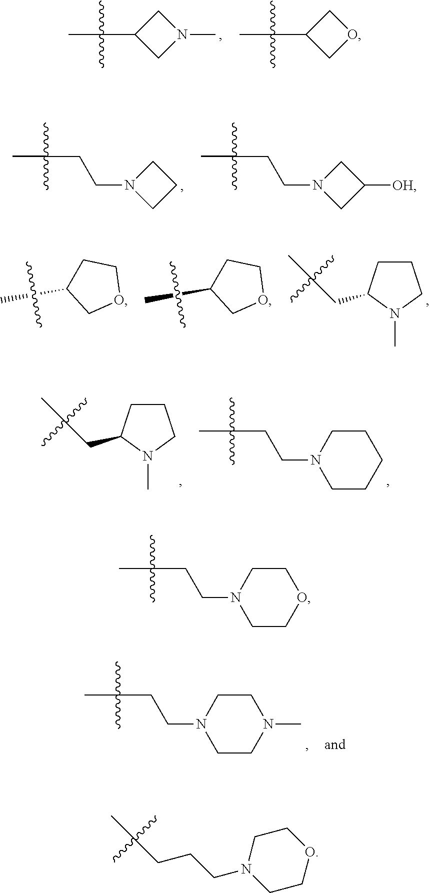

In some embodiments, R₄ is chosen from —CH═CH₂,

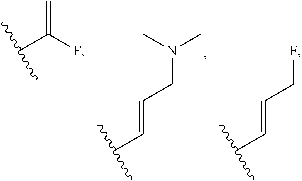

—C≡C—CH₃, —CH═CH—CH₃, and

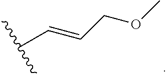 [SMILES: */C=C/COC]

In one embodiment, R₄ is —CH═CH₂. In one embodiment, R₄ is

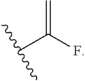 [SMILES: *C(=C)F]

In one embodiment, R₄ is

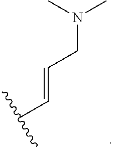 [SMILES: */C=C/CN(C)C]

In one embodiment, R₄ is

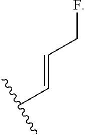 [SMILES: */C=C/CF]

In one embodiment, R₄ is —C≡C—CH₃. In one embodiment, R₄ is —CH═CH—CH₃. In one embodiment, R₄ is

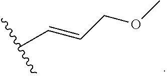 [SMILES: */C=C/COC]

In one embodiment, Q is “NH” or “O”, with the proviso that when Q is “O”, A is not

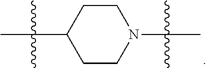 [SMILES: *C1CCN(*)CC1]

In some embodiments, ring A is chosen from

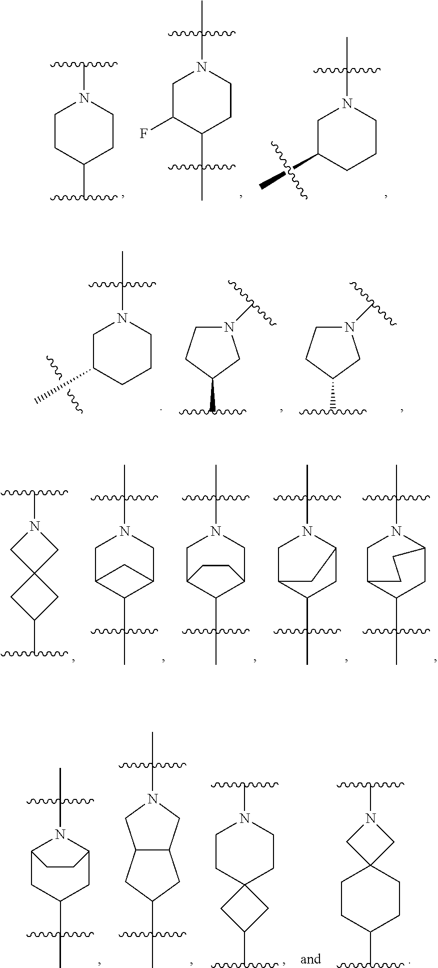

In one embodiment, ring A is

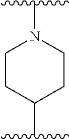 [SMILES: *C1CCN(*)CC1]

with the proviso that when Q is “O”, A is not

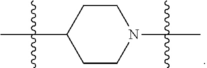 [SMILES: *C1CCN(*)CC1]

In one embodiment, ring A is

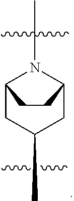

In one embodiment, ring A is

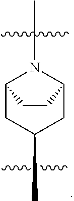

In some embodiments, provided herein is a compound, or pharmaceutically acceptable salt thereof, chosen from the compounds listed in Table 1.

TABLE 1
Exemplary Compounds of the Present Disclosure

| Com- | | |
|---|---|---|
| pound | | |
| No. | Chemical Structure | IUPAC Nomenclature |
| 1 | 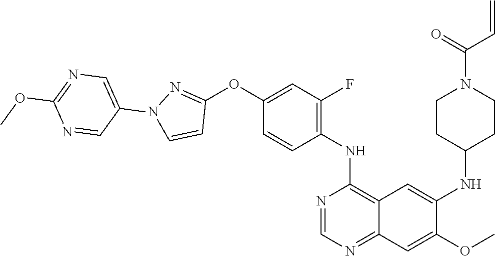 | 1-(4-((4-((2-fluoro-4-((1-(2- methoxypyrimidin- 5-yl)-1H-pyrazol- 3-yl)oxy)phenyl)amino)-7- methoxyquinazolin-6- yl)amino)piperidin- 1-yl)prop-2-en-1- one |
| 2 | 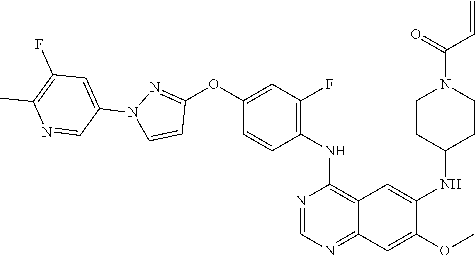 | 1-(4-((4-((2-fluoro- 4-((1-(5-fluoro-6- methylpyridin-3- yl)-1H-pyrazol-3- yl)oxy)phenyl)amino)-7- methoxyquinazolin-6- yl)amino)piperidin- 1-yl)prop-2-en-1- one |
| 3 | 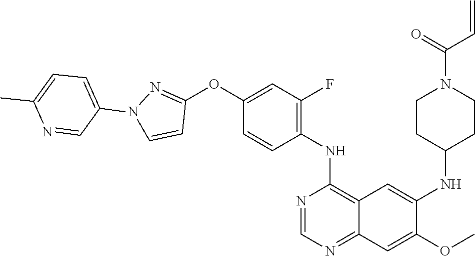 | 1-(4-((4-((2-fluoro-4-((1-(6- methylpyridin-3- yl)-1H-pyrazol-3- yl)oxy)phenyl)amino)-7- methoxyquinazolin-6- yl)amino)piperidin- 1-yl)prop-2-en-1- one |
| 4 | 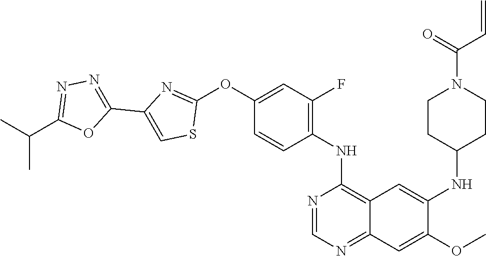 [SMILES: C=CC(=O)N1CCC(Nc2cc3c(Nc4ccc(Oc5nc(-c6nnc(C(C)C)o6)cs5)cc4F)ncnc3cc2OC)CC1] | 1-(4-((4-((2-fluoro- 4-((4-(5-isopropyl- 1,3,4-oxadiazol-2- yl)thiazol-2- yl)oxy)phenyl)amino)-7- methoxyquinazolin-6- yl)amino)piperidin- 1-yl)prop-2-en-1- one |
| 5 | 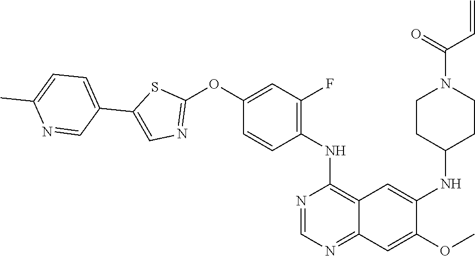 | 1-(4-((4-((2-fluoro-4-((5-(6- methylpyridin-3-yl)thiazol-2- yl)oxy)phenyl)amino)-7- methoxyquinazolin-6- yl)amino)piperidin- 1-yl)prop-2-en-1- one |
| 6 | 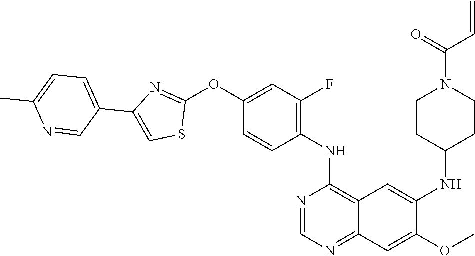 | 1-(4-((4-((2-fluoro-4-((4-(6- methylpyridin-3-yl)thiazol-2- yl)oxy)phenyl)amino)-7- methoxyquinazolin-6- yl)amino)piperidin- 1-yl)prop-2-en-1- one |
| 7 | 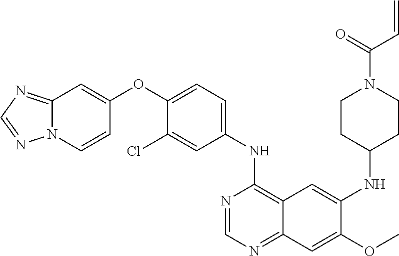 [SMILES: C=CC(=O)N1CCC(Nc2cc3c(Nc4ccc(Oc5ccn6ncnc6c5)c(Cl)c4)ncnc3cc2OC)CC1] | 1-(4-((4-((4- ([1,2,4]triazolo[1,5- a]pyridin-7-yloxy)-3- chlorophenyl)amino)-7- methoxyquinazolin-6- yl)amino)piperidin- 1-yl)prop-2-en-1- one |
| 8 | 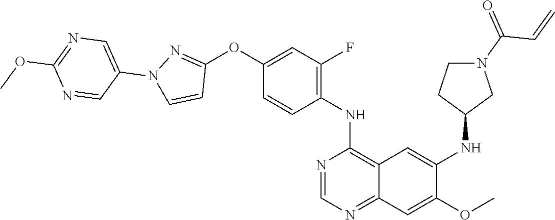 [SMILES: C=CC(=O)N1CC[C@H](Nc2cc3c(Nc4ccc(Oc5ccn(-c6cnc(OC)nc6)n5)cc4F)ncnc3cc2OC)C1] | (S)-1-(3-((4-((2- fluoro-4-((1-(2- methoxypyrimidin- 5-yl)-1H-pyrazol- 3-yl)oxy)phenyl)amino)-7- methoxyquinazolin-6- yl)amino)pyrrolidin- 1-yl)prop-2-en-1- one |
| 9 | 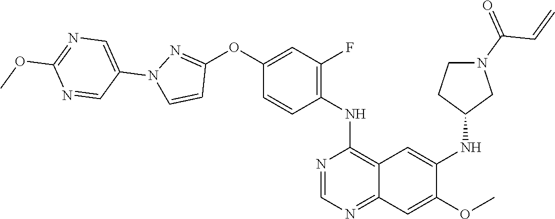 [SMILES: C=CC(=O)N1CC[C@@H](Nc2cc3c(Nc4ccc(Oc5ccn(-c6cnc(OC)nc6)n5)cc4F)ncnc3cc2OC)C1] | (R)-1-(3-((4-((2- fluoro-4-((1-(2- methoxypyrimidin- 5-yl)-1H-pyrazol- 3-yl)oxy)phenyl)amino)-7- methoxyquinazolin-6- yl)amino)pyrrolidin- 1-yl)prop-2-en-1- one |
| 10 | 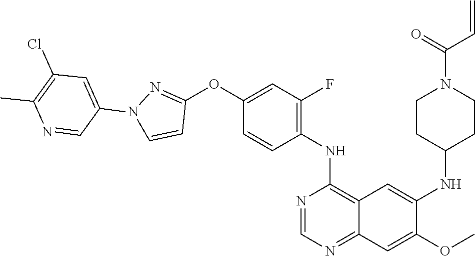 | 1-(4-((4-((4-((1-(5-chloro-6- methylpyridin-3-yl)- 1H-pyrazol-3- yl)oxy)-2- fluorophenyl)amino)-7- methoxyquinazolin-6- yl)amino)piperidin- 1-yl)prop-2-en-1- one |
| 11 | 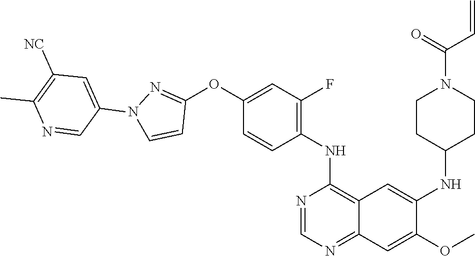 | 5-(3-(4-((6-((1- acryloylpiperidin-4- yl)amino)-7- methoxyquinazolin-4- yl)amino)-3- fluorophenoxy)-1H- pyrazol-1-yl)-2- methylnicotinonitrile |
| 12 | 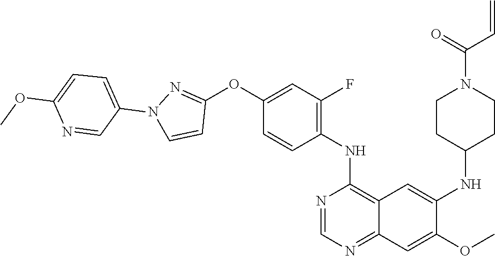 | 1-(4-((4-((2-fluoro-4-((1-(6- methoxypyridin-3- yl)-1H-pyrazol-3- yl)oxy)phenyl)amino)-7- methoxyquinazolin-6- yl)amino)piperidin- 1-yl)prop-2-en-1- one |
| 13 | 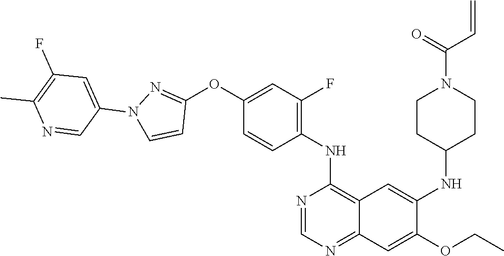 [SMILES: C=CC(=O)N1CCC(Nc2cc3c(Nc4ccc(Oc5ccn(-c6cnc(C)c(F)c6)n5)cc4F)ncnc3cc2OCC)CC1] | 1-(4-((7-ethoxy-4- ((2-fluoro-4-((1-(5- fluoro-6-methylpyridin- 3-yl)-1H- pyrazol-3- yl)oxy)phenyl) amino)quinazolin-6- yl)amino)piperidin- 1-yl)prop-2-en-1- one |
| 14 | 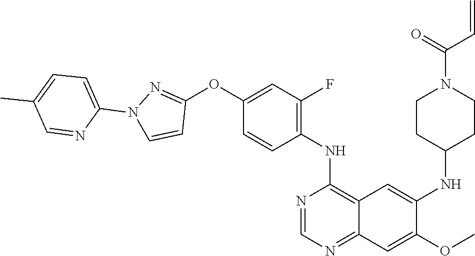 | 1-(4-((4-((2-fluoro-4-((1-(5- methylpyridin-2-yl)- 1H-pyrazol-3- yl)oxy)phenyl)amino)-7- methoxyquinazolin-6- yl)amino)piperidin- 1-yl)prop-2-en-1- one |
| 15 | 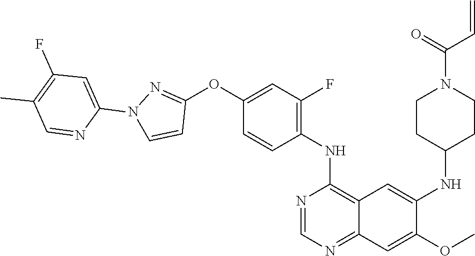 | 1-(4-((4-((2-fluoro- 4-((1-(4-fluoro-5- methylpyridin-2-yl)- 1H-pyrazol-3- yl)oxy)phenyl)amino)-7- methoxyquinazolin-6- yl)amino)piperidin- 1-yl)prop-2-en-1- one |
| 16 | 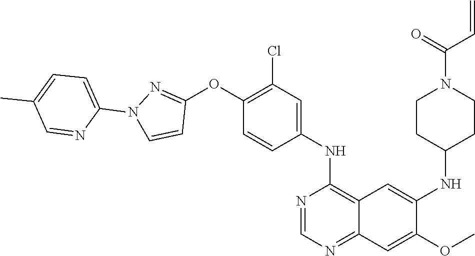 | 1-(4-((4-((3-chloro-4-((1-(5- methylpyridin-2- yl)-1H-pyrazol-3- yl)oxy)phenyl)amino)-7- methoxyquinazolin-6- yl)amino)piperidin- 1-yl)prop-2-en-1- one |
| 17 | 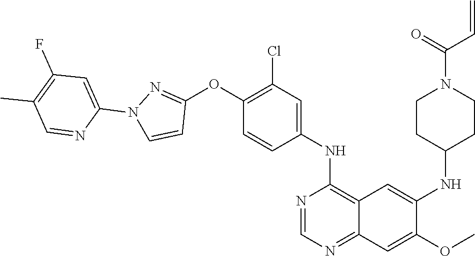 | 1-(4-((4-((3-chloro- 4-((1-(4-fluoro-5- methylpyridin-2-yl)- 1H-pyrazol-3- yl)oxy)phenyl)amino)-7- methoxyquinazolin-6- yl)amino)piperidin- 1-yl)prop-2-en-1- one |
| 18 | 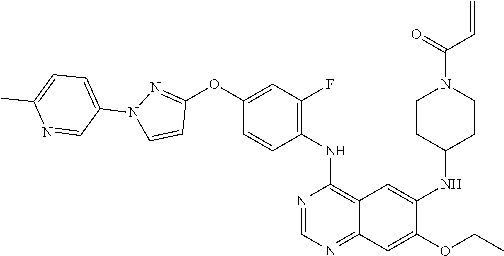 [SMILES: C=CC(=O)N1CCC(Nc2cc3c(Nc4ccc(Oc5ccn(-c6ccc(C)nc6)n5)cc4F)ncnc3cc2OCC)CC1] | 1-(4-((7-ethoxy- 4-((2-fluoro-4-((1-(6- methylpyridin-3-yl)- 1H-pyrazol-3- yl)oxy)phenyl)amino) quinazolin-6- yl)amino)piperidin- 1-yl)prop-2-en-1- one |
| 19 | 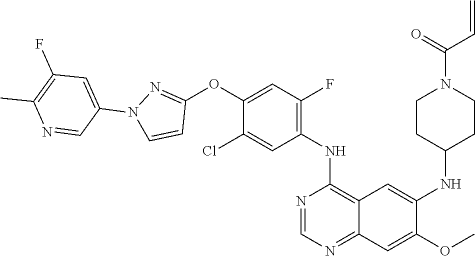 | 1-(4-((4-((5-chloro- 2-fluoro-4-((1-(5- fluoro-6-methylpyridin- 3-yl)-1H- pyrazol-3-yl)oxy) phenyl)amino)-7- methoxyquinazolin-6- yl)amino)piperidin- 1-yl)prop-2-en-1- one |
| 20 | 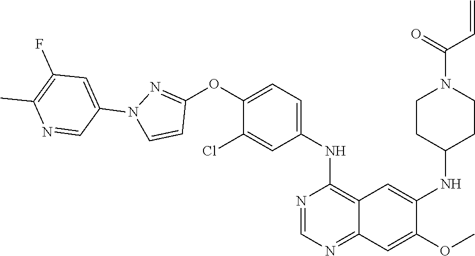 | 1-(4-((4-((3-chloro- 4-((1-(5-fluoro-6- methylpyridin-3- yl)-1H-pyrazol-3- yl)oxy)phenyl)amino)-7- methoxyquinazolin-6- yl)amino)piperidin- 1-yl)prop-2-en-1- one |
| 21 | 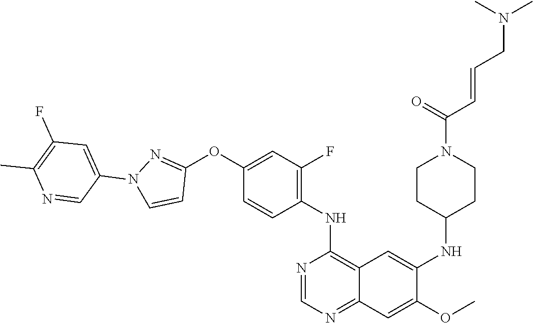 | (E)-4-(dimethylamino)- 1-(4-((4-((2- fluoro-4-((1-(5-fluoro-6- methylpyridin-3-yl)- 1H-pyrazol-3- yl)oxy)phenyl)amino)-7- methoxyquinazolin-6- yl)amino)piperidin- 1-yl)but-2-en-1- one |
| 22 | 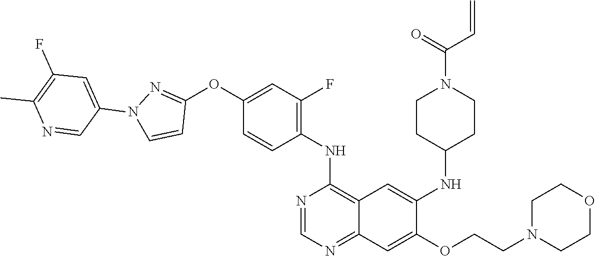 [SMILES: C=CC(=O)N1CCC(Nc2cc3c(Nc4ccc(Oc5ccn(-c6cnc(C)c(F)c6)n5)cc4F)ncnc3cc2OCCN2CCOCC2)CC1] | 1-(4-((4-((2-fluoro- 4-((1-(5-fluoro-6- methylpyridin-3-yl)- 1H-pyrazol-3- yl)oxy)phenyl)amino)-7-(2- morpholinoethoxy) quinazolin-6- yl)amino)piperidin- 1-yl)prop-2-en-1- one |
| 23 | 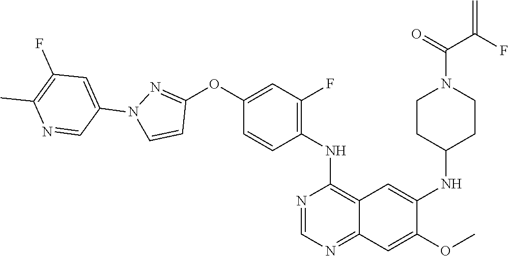 [SMILES: C=C(F)C(=O)N1CCC(Nc2cc3c(Nc4ccc(Oc5ccn(-c6cnc(C)c(F)c6)n5)cc4F)ncnc3cc2OC)CC1] | 2-fluoro-1-(4-((4- ((2-fluoro-4-((1-(5- fluoro-6-methylpyridin- 3-yl)-1H- pyrazol-3-yl)oxy) phenyl)amino)-7- methoxyquinazolin-6- yl)amino)piperidin- 1-yl)prop-2-en-1- one |
| 24 | 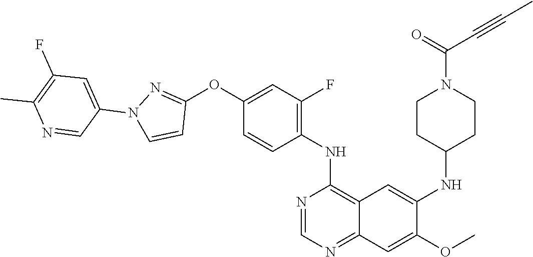 [SMILES: CC#CC(=O)N1CCC(Nc2cc3c(Nc4ccc(Oc5ccn(-c6cnc(C)c(F)c6)n5)cc4F)ncnc3cc2OC)CC1] | 1-(4-((4-((2-fluoro- 4-((1-(5-fluoro-6- methylpyridin-3- yl)-1H-pyrazol-3- yl)oxy)phenyl)amino)-7- methoxyquinazolin-6- yl)amino)piperidin- 1-yl)but-2-yn-1- one |
| 25 | 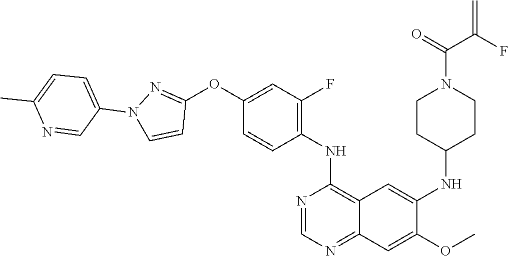 [SMILES: C=C(F)C(=O)N1CCC(Nc2cc3c(Nc4ccc(Oc5ccn(-c6ccc(C)nc6)n5)cc4F)ncnc3cc2OC)CC1] | 2-fluoro-1-(4-((4- ((2-fluoro-4-((1-(6- methylpyridin-3- yl)-1H-pyrazol-3- yl)oxy)phenyl)amino)-7- methoxyquinazolin-6- yl)amino)piperidin- 1-yl)prop-2-en-1- one |
| 26 | 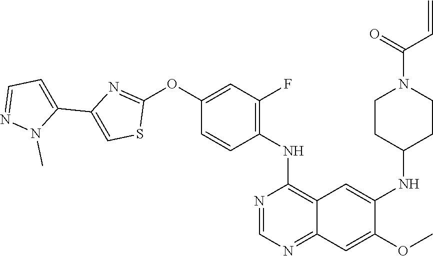 [SMILES: C=CC(=O)N1CCC(Nc2cc3c(Nc4ccc(Oc5nc(-c6ccnn6C)cs5)cc4F)ncnc3cc2OC)CC1] | 1-(4-((4-((2-fluoro- 4-((4-(1-methyl- 1H-pyrazol-5-yl)thiazol-2- yl)oxy)phenyl)amino)-7- methoxyquinazolin-6- yl)amino)piperidin- 1-yl)prop-2-en-1- one |
| 27 | 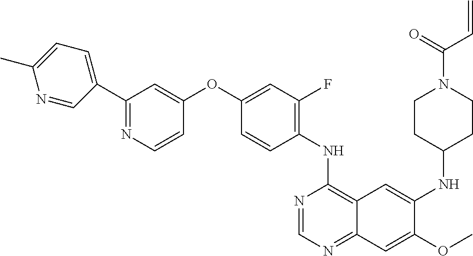 | 1-(4-((4-((2-fluoro- 4-((6′-methyl-[2,3′- bipyridin]-4-yl)oxy) phenyl)amino)-7- methoxyquinazolin-6- yl)amino)piperidin- 1-yl)prop-2-en-1- one |
| 28 | 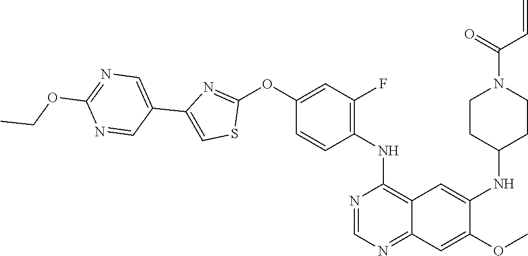 [SMILES: C=CC(=O)N1CCC(Nc2cc3c(Nc4ccc(Oc5nc(-c6cnc(OCC)nc6)cs5)cc4F)ncnc3cc2OC)CC1] | 1-(4-((4-((4-((4-(2- ethoxypyrimidin-5- yl)thiazol-2-yl)oxy)-2- fluorophenyl)amino)-7- methoxyquinazolin-6- yl)amino)piperidin- 1-yl)prop-2-en-1- one |
| 29 | 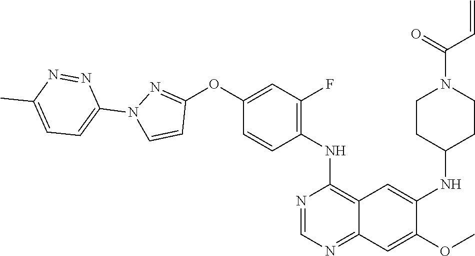 | 1-(4-((4-((2-fluoro-4-((1-(6- methylpyridazin-3- yl)-1H-pyrazol-3- yl)oxy)phenyl)amino)-7- methoxyquinazolin-6- yl)amino)piperidin- 1-yl)prop-2-en-1- one |
| 30 | 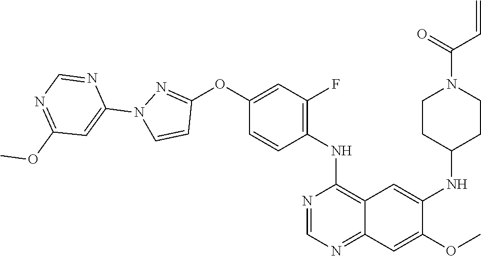 | 1-(4-((4-((2-fluoro-4-((1-(6- methoxypyrimidin-4- yl)-1H-pyrazol- 3-yl)oxy)phenyl)amino)-7- methoxyquinazolin-6- yl)amino)piperidin- 1-yl)prop-2-en-1- one |
| 31 | 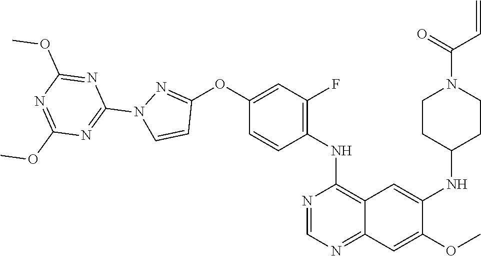 | 1-(4-((4-((4-((1- (4,6-dimethoxy- 1,3,5-triazin-2-yl)- 1H-pyrazol-3- yl)oxy)-2- fluorophenyl)amino)-7- methoxyquinazolin-6- yl)amino)piperidin- 1-yl)prop-2-en-1- one |
| 32 | 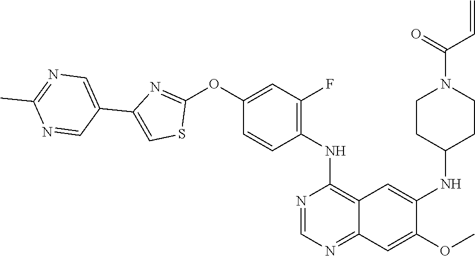 | 1-(4-((4-((2-fluoro-4-((4-(2- methylpyrimidin- 5-yl)thiazol-2- yl)oxy)phenyl)amino)-7- methoxyquinazolin-6- yl)amino)piperidin- 1-yl)prop-2-en-1- one |
| 33 | 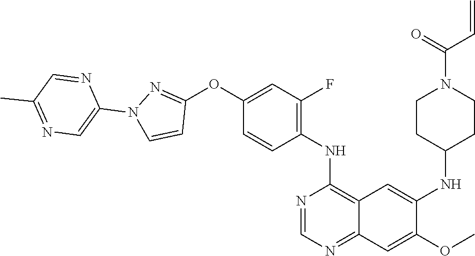 | 1-(4-((4-((2-fluoro-4-((1-(5- methylpyrazin-2-yl)- 1H-pyrazol-3- yl)oxy)phenyl)amino)-7- methoxyquinazolin-6- yl)amino)piperidin- 1-yl)prop-2-en-1- one |
| 34 | 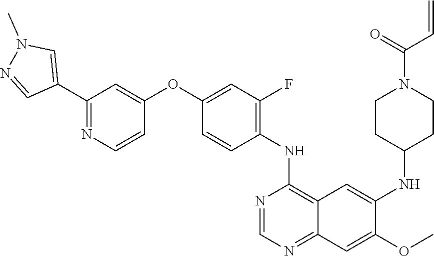 [SMILES: C=CC(=O)N1CCC(Nc2cc3c(Nc4ccc(Oc5ccnc(-c6cnn(C)c6)c5)cc4F)ncnc3cc2OC)CC1] | 1-(4-((4-((2-fluoro- 4-((2-(1-methyl- 1H-pyrazol-4-yl)pyridin-4- yl)oxy)phenyl)amino)-7- methoxyquinazolin-6- yl)amino)piperidin- 1-yl)prop-2-en-1- one |
| 35 | 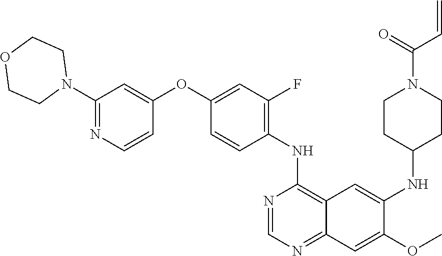 [SMILES: C=CC(=O)N1CCC(Nc2cc3c(Nc4ccc(Oc5ccnc(N6CCOCC6)c5)cc4F)ncnc3cc2OC)CC1] | 1-(4-((4-((2-fluoro-4-((2- morpholinopyridin-4- yl)oxy)phenyl)amino)-7- methoxyquinazolin-6- yl)amino)piperidin- 1-yl)prop-2-en-1- one |
| 36 | 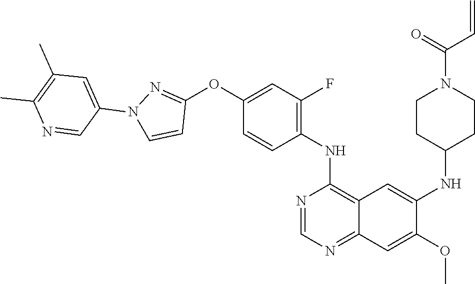 [SMILES: C=CC(=O)N1CCC(Nc2cc3c(Nc4ccc(Oc5ccn(-c6cnc(C)c(C)c6)n5)cc4F)ncnc3cc2OC)CC1] | 1-(4-((4-((4-((1- (5,6-dimethylpyridin- 3-yl)-1H-pyrazol- 3-yl)oxy)-2- fluorophenyl)amino)-7- methoxyquinazolin-6- yl)amino)piperidin- 1-yl)prop-2-en-1- one |
| 37 | 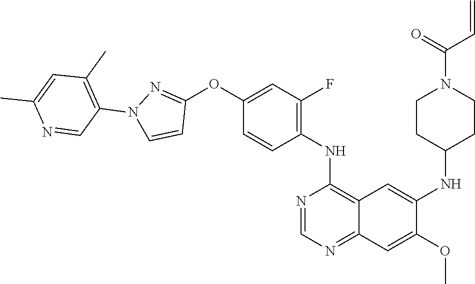 [SMILES: C=CC(=O)N1CCC(Nc2cc3c(Nc4ccc(Oc5ccn(-c6cnc(C)cc6C)n5)cc4F)ncnc3cc2OC)CC1] | 1-(4-((4-((4-((1- (4,6-dimethylpyridin- 3-yl)-1H-pyrazol- 3-yl)oxy)-2- fluorophenyl)amino)-7- methoxyquinazolin-6- yl)amino)piperidin- 1-yl)prop-2-en-1- one |
| 38 | 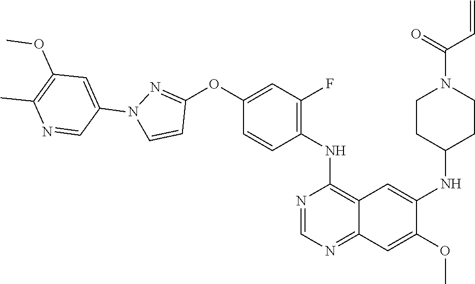 [SMILES: C=CC(=O)N1CCC(Nc2cc3c(Nc4ccc(Oc5ccn(-c6cnc(C)c(OC)c6)n5)cc4F)ncnc3cc2OC)CC1] | 1-(4-((4-((2-fluoro- 4-((1-(5-methoxy- 6-methylpyridin-3- yl)-1H-pyrazol-3- yl)oxy)phenyl)amino)-7- methoxyquinazolin-6- yl)amino)piperidin- 1-yl)prop-2-en-1- one |
| 39 | 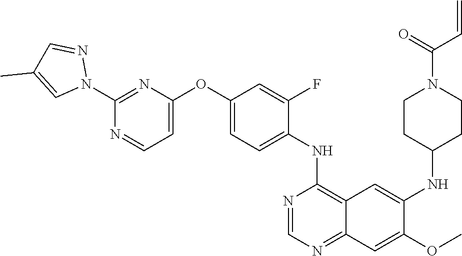 | 1-(4-((4-((2-fluoro- 4-((2-(4-methyl- 1H-pyrazol-1- yl)pyrimidin-4- yl)oxy)phenyl)amino)-7- methoxyquinazolin-6- yl)amino)piperidin- 1-yl)prop-2-en-1- one |
| 40 | 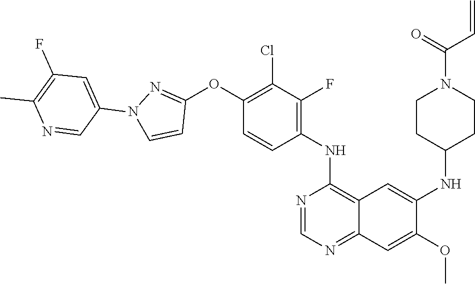 [SMILES: C=CC(=O)N1CCC(Nc2cc3c(Nc4ccc(Oc5ccn(-c6cnc(C)c(F)c6)n5)c(Cl)c4F)ncnc3cc2OC)CC1] | 1-(4-((4-((3-chloro- 2-fluoro-4-((1-(5- fluoro-6-methylpyridin- 3-yl)-1H- pyrazol-3-yl)oxy) phenyl)amino)-7- methoxyquinazolin-6- yl)amino)piperidin- 1-yl)prop-2-en-1- one |
| 41 | 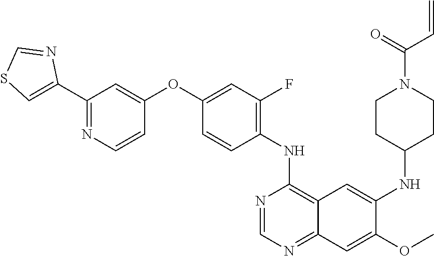 [SMILES: C=CC(=O)N1CCC(Nc2cc3c(Nc4ccc(Oc5ccnc(-c6cscn6)c5)cc4F)ncnc3cc2OC)CC1] | 1-(4-((4-((2-fluoro- 4-((2-(thiazol-4- yl)pyridin-4-yl)oxy) phenyl)amino)-7- methoxyquinazolin-6- yl)amino)piperidin- 1-yl)prop-2-en-1- one |
| 42 | 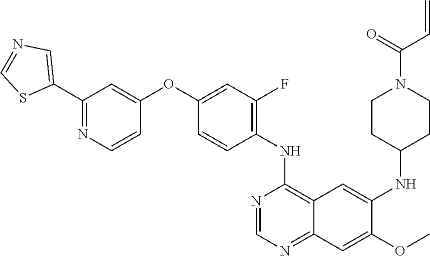 [SMILES: C=CC(=O)N1CCC(Nc2cc3c(Nc4ccc(Oc5ccnc(-c6cncs6)c5)cc4F)ncnc3cc2OC)CC1] | 1-(4-((4-((2-fluoro- 4-((2-(thiazol-5- yl)pyridin-4-yl)oxy) phenyl)amino)-7- methoxyquinazolin-6- yl)amino)piperidin- 1-yl)prop-2-en-1- one |
| 43 | 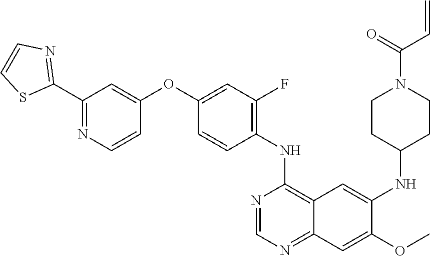 [SMILES: C=CC(=O)N1CCC(Nc2cc3c(Nc4ccc(Oc5ccnc(-c6nccs6)c5)cc4F)ncnc3cc2OC)CC1] | 1-(4-((4-((2-fluoro- 4-((2-(thiazol-2- yl)pyridin-4-yl)oxy) phenyl)amino)-7- methoxyquinazolin-6- yl)amino)piperidin- 1-yl)prop-2-en-1- one |
| 44 | 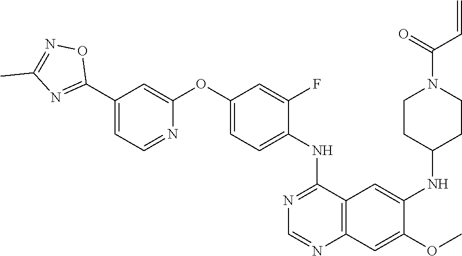 | 1-(4-((4-((2-fluoro- 4-((4-(3-methyl- 1,2,4-oxadiazol- 5-yl)pyridin-2- yl)oxy)phenyl)amino)-7- methoxyquinazolin-6- yl)amino)piperidin- 1-yl)prop-2-en-1- one |
| 45 | 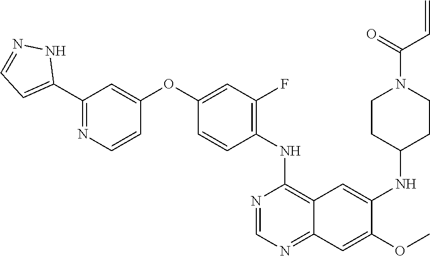 [SMILES: C=CC(=O)N1CCC(Nc2cc3c(Nc4ccc(Oc5ccnc(-c6ccn[nH]6)c5)cc4F)ncnc3cc2OC)CC1] | 1-(4-((4-((4-((2- (1H-pyrazol-5- yl)pyridin-4-yl)oxy)-2- fluorophenyl)amino)-7- methoxyquinazolin-6- yl)amino)piperidin- 1-yl)prop-2-en-1- one |
| 46 | 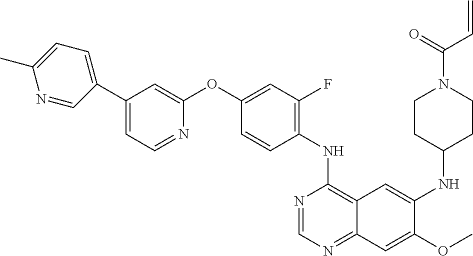 | 1-(4-((4-((2-fluoro- 4-((6-methyl-[3,4′- bipyridin]-2′-yl)oxy) phenyl)amino)-7- methoxyquinazolin-6- yl)amino)piperidin- 1-yl)prop-2-en-1- one |
| 47 | 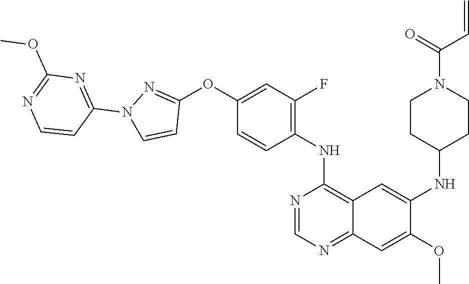 [SMILES: C=CC(=O)N1CCC(Nc2cc3c(Nc4ccc(Oc5ccn(-c6ccnc(OC)n6)n5)cc4F)ncnc3cc2OC)CC1] | 1-(4-((4-((2-fluoro- 4-((1-(2- methoxypyrimidin- 4-yl)-1H-pyrazol- 3-yl)oxy)phenyl)amino)-7- methoxyquinazolin-6- yl)amino)piperidin- 1-yl)prop-2-en-1- one |
| 48 | 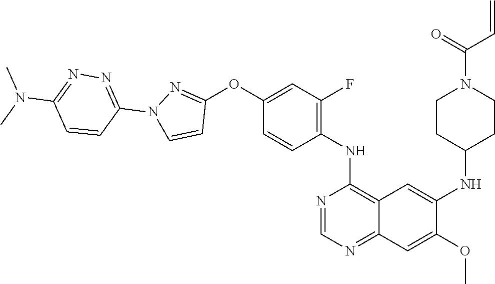 [SMILES: C=CC(=O)N1CCC(Nc2cc3c(Nc4ccc(Oc5ccn(-c6ccc(N(C)C)nn6)n5)cc4F)ncnc3cc2OC)CC1] | 1-(4-((4-((4-((1-(6- (dimethylamino) pyridazin-3-yl)-1H- pyrazol-3-yl)oxy)-2- fluorophenyl)amino)-7- methoxyquinazolin-6- yl)amino)piperidin- 1-yl)prop-2-en-1- one |
| 49 | 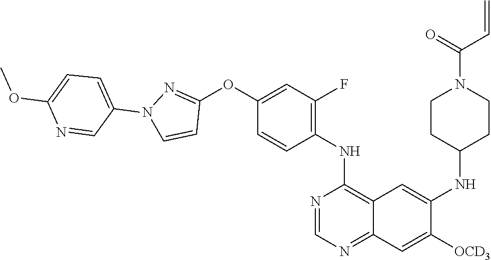 | 1-(4-((4-((2-fluoro-4-((1-(6- methoxypyridin-3- yl)-1H-pyrazol-3- yl)oxy)phenyl)amino)- 7-(methoxy- d3)quinazolin-6-yl) amino)piperidin- 1-yl)prop-2-en-1-one |
| 50 | 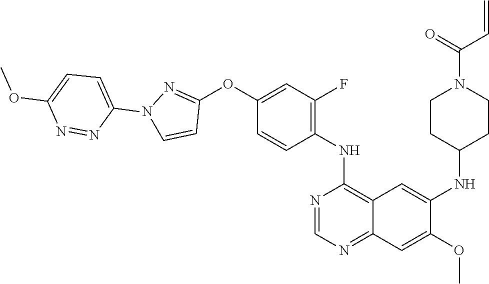 [SMILES: C=CC(=O)N1CCC(Nc2cc3c(Nc4ccc(Oc5ccn(-c6ccc(OC)nn6)n5)cc4F)ncnc3cc2OC)CC1] | 1-(4-((4-((2-fluoro-4-((1-(6- methoxypyridazin- 3-yl)-1H-pyrazol- 3-yl)oxy)phenyl)amino)-7- methoxyquinazolin-6- yl)amino)piperidin- 1-yl)prop-2-en-1- one |
| 51 | 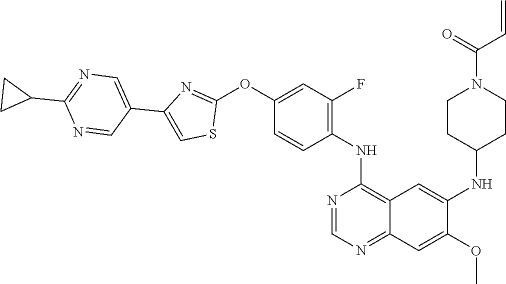 [SMILES: C=CC(=O)N1CCC(Nc2cc3c(Nc4ccc(Oc5nc(-c6cnc(C7CC7)nc6)cs5)cc4F)ncnc3cc2OC)CC1] | 1-(4-((4-((4-((4-(2- cyclopropylpyrimidin- 5-yl)thiazol-2- yl)oxy)-2- fluorophenyl)amino)-7- methoxyquinazolin-6- yl)amino)piperidin- 1-yl)prop-2-en-1- one |
| 52 | 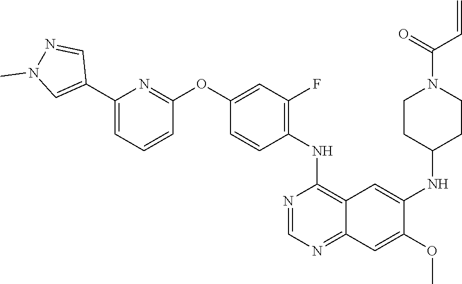 | 1-(4-((4-((2-fluoro- 4-((6-(1-methyl- 1H-pyrazol-4-yl)pyridin-2- yl)oxy)phenyl)amino)-7- methoxyquinazolin-6- yl)amino)piperidin- 1-yl)prop-2-en-1- one |
| 53 | 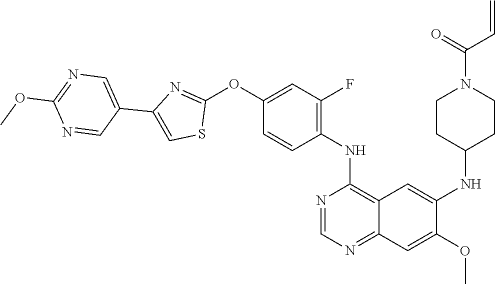 [SMILES: C=CC(=O)N1CCC(Nc2cc3c(Nc4ccc(Oc5nc(-c6cnc(OC)nc6)cs5)cc4F)ncnc3cc2OC)CC1] | 1-(4-((4-((2-fluoro-4-((4-(2- methoxypyrimidin- 5-yl)thiazol-2- yl)oxy)phenyl)amino)-7- methoxyquinazolin-6- yl)amino)piperidin- 1-yl)prop-2-en-1- one |
| 54 | 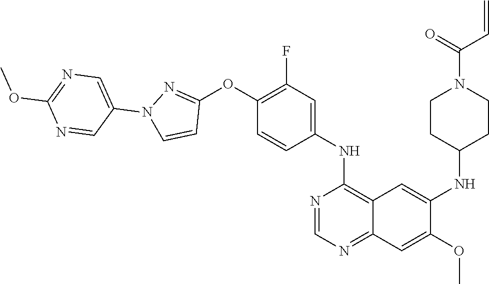 [SMILES: C=CC(=O)N1CCC(Nc2cc3c(Nc4ccc(Oc5ccn(-c6cnc(OC)nc6)n5)c(F)c4)ncnc3cc2OC)CC1] | 1-(4-((4-((3-fluoro-4-((1-(2- methoxypyrimidin- 5-yl)-1H-pyrazol- 3-yl)oxy)phenyl)amino)-7- methoxyquinazolin-6- yl)amino)piperidin- 1-yl)prop-2-en-1- one |
| 55 | 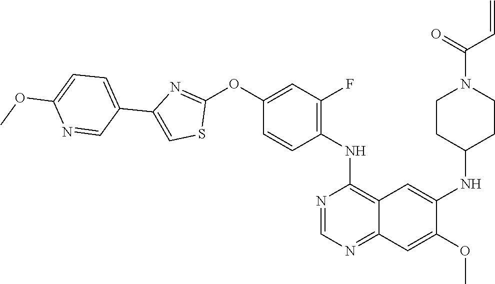 [SMILES: C=CC(=O)N1CCC(Nc2cc3c(Nc4ccc(Oc5nc(-c6ccc(OC)nc6)cs5)cc4F)ncnc3cc2OC)CC1] | 1-(4-((4-((2-fluoro-4-((4-(6- methoxypyridin- 3-yl)thiazol-2- yl)oxy)phenyl)amino)-7- methoxyquinazolin-6- yl)amino)piperidin- 1-yl)prop-2-en-1- one |
| 56 | 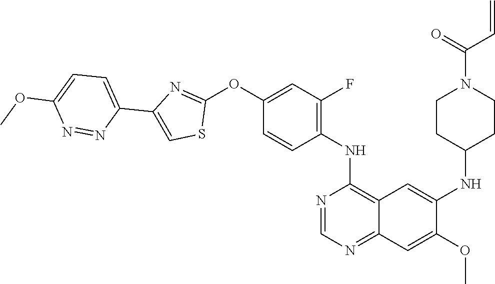 [SMILES: C=CC(=O)N1CCC(Nc2cc3c(Nc4ccc(Oc5nc(-c6ccc(OC)nn6)cs5)cc4F)ncnc3cc2OC)CC1] | 1-(4-((4-((2-fluoro-4-((4-(6- methoxypyridazin- 3-yl)thiazol-2- yl)oxy)phenyl)amino)-7- methoxyquinazolin-6- yl)amino)piperidin- 1-yl)prop-2-en-1- one |
| 57 | 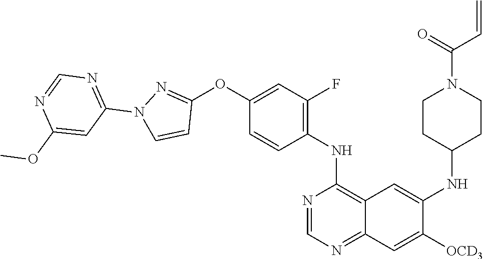 | 1-(4-((4-((2-fluoro-4-((1-(6- methoxypyrimidin- 4-yl)-1H-pyrazol- 3-yl)oxy)phenyl) amino)-7-(methoxy- d3)quinazolin-6-yl) amino)piperidin- 1-yl)prop-2-en-1-one |
| 58 | 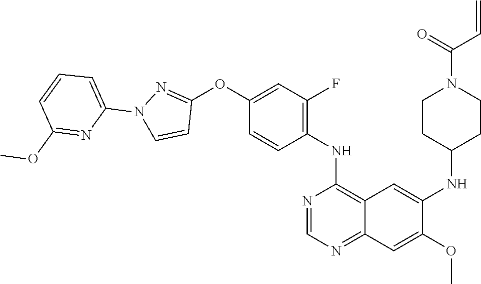 [SMILES: C=CC(=O)N1CCC(Nc2cc3c(Nc4ccc(Oc5ccn(-c6cccc(OC)n6)n5)cc4F)ncnc3cc2OC)CC1] | 1-(4-((4-((2-fluoro-4-((1-(6- methoxypyridin-2- yl)-1H-pyrazol-3- yl)oxy)phenyl)amino)-7- methoxyquinazolin-6- yl)amino)piperidin- 1-yl)prop-2-en-1- one |
| 59 | 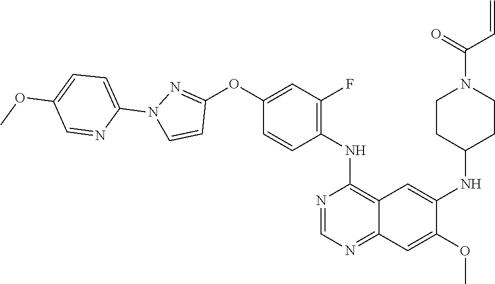 [SMILES: C=CC(=O)N1CCC(Nc2cc3c(Nc4ccc(Oc5ccn(-c6ccc(OC)cn6)n5)cc4F)ncnc3cc2OC)CC1] | 1-(4-((4-((2-fluoro-4-((1-(5- methoxypyridin-2- yl)-1H-pyrazol-3- yl)oxy)phenyl)amino)-7- methoxyquinazolin-6- yl)amino)piperidin- 1-yl)prop-2-en-1- one |
| 60 | 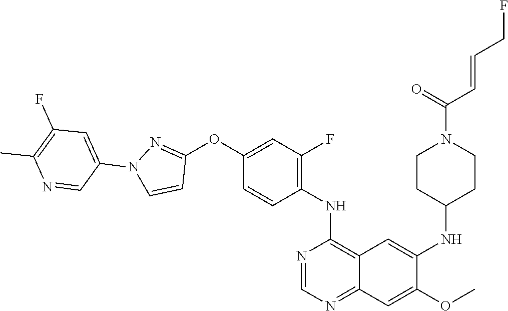 [SMILES: COc1cc2ncnc(Nc3ccc(Oc4ccn(-c5cnc(C)c(F)c5)n4)cc3F)c2cc1NC1CCN(C(=O)/C=C/CF)CC1] | (E)-4-fluoro-1-(4- ((4-((2-fluoro-4-((1- (5-fluoro-6- methylpyridin-3-yl)-1H- pyrazol-3-yl)oxy) phenyl)amino)-7- methoxyquinazolin-6- yl)amino)piperidin- 1-yl)but-2-en-1- one |
| 61 | 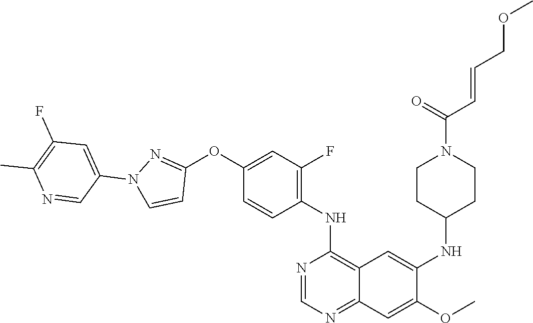 | (E)-1-(4-((4-((2- fluoro-4-((1-(5- fluoro-6-methylpyridin- 3-yl)-1H- pyrazol-3-yl)oxy) phenyl)amino)-7- methoxyquinazolin-6- yl)amino)piperidin-1-yl)-4- methoxybut-2-en-1-one |
| 62 | 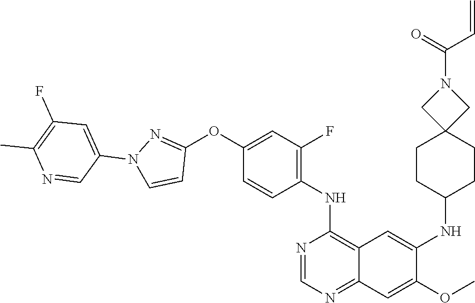 [SMILES: C=CC(=O)N1CC2(CCC(Nc3cc4c(Nc5ccc(Oc6ccn(-c7cnc(C)c(F)c7)n6)cc5F)ncnc4cc3OC)CC2)C1] | 1-(7-((4-((2-fluoro- 4-((1-(5-fluoro-6- methylpyridin-3-yl)- 1H-pyrazol-3- yl)oxy)phenyl)amino)-7- methoxyquinazolin- 6-yl)amino)-2- azaspiro[3.5]nonan- 2-yl)prop-2-en- 1-one |
| 63 | 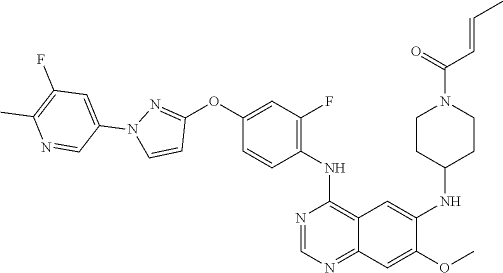 [SMILES: C/C=C/C(=O)N1CCC(Nc2cc3c(Nc4ccc(Oc5ccn(-c6cnc(C)c(F)c6)n5)cc4F)ncnc3cc2OC)CC1] | (E)-1-(4-((4- ((2-fluoro-4-((1-(5- fluoro-6-methylpyridin- 3-yl)-1H- pyrazol-3-yl)oxy) phenyl)amino)-7- methoxyquinazolin-6- yl)amino)piperidin- 1-yl)but-2-en-1- one |
| 64 | 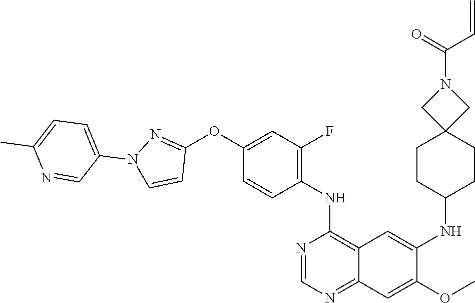 [SMILES: C=CC(=O)N1CC2(CCC(Nc3cc4c(Nc5ccc(Oc6ccn(-c7ccc(C)nc7)n6)cc5F)ncnc4cc3OC)CC2)C1] | 1-(7-((4-((2-fluoro-4-((1-(6- methylpyridin-3- yl)-1H-pyrazol-3- yl)oxy)phenyl)amino)-7- methoxyquinazolin- 6-yl)amino)-2- azaspiro[3.5]nonan- 2-yl)prop-2-en- 1-one |
| 65 | 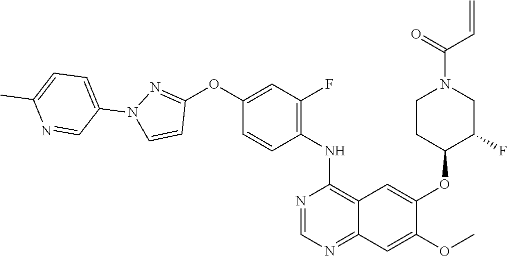 [SMILES: C=CC(=O)N1CC[C@H](Oc2cc3c(Nc4ccc(Oc5ccn(-c6ccc(C)nc6)n5)cc4F)ncnc3cc2OC)[C@@H](F)C1] | 1-((3S,4S)-3-fluoro- 4-((4-((2-fluoro- 4-((1-(6-methylpyridin- 3-yl)-1H- pyrazol-3-yl)oxy) phenyl)amino)-7- methoxyquinazolin-6- yl)oxy)piperidin- 1-yl)prop-2-en-1- one |
| 66 | 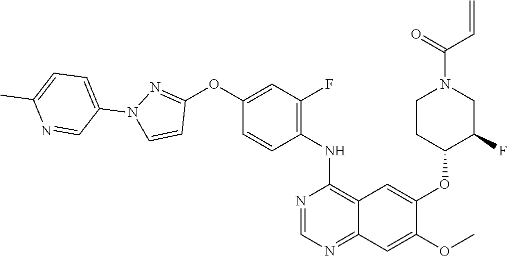 [SMILES: C=CC(=O)N1CC[C@@H](Oc2cc3c(Nc4ccc(Oc5ccn(-c6ccc(C)nc6)n5)cc4F)ncnc3cc2OC)[C@H](F)C1] | 1-((3R,4R)-3-fluoro- 4-((4-((2-fluoro- 4-((1-(6-methylpyridin- 3-yl)-1H- pyrazol-3-yl)oxy) phenyl)amino)-7- methoxyquinazolin-6- yl)oxy)piperidin- 1-yl)prop-2-en-1- one |
| 67 | 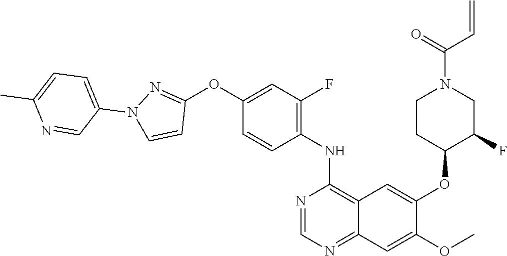 [SMILES: C=CC(=O)N1CC[C@H](Oc2cc3c(Nc4ccc(Oc5ccn(-c6ccc(C)nc6)n5)cc4F)ncnc3cc2OC)[C@H](F)C1] | 1-((3R,4S)-3-fluoro- 4-((4-((2-fluoro- 4-((1-(6-methylpyridin- 3-yl)-1H- pyrazol-3-yl)oxy) phenyl)amino)-7- methoxyquinazolin-6- yl)oxy)piperidin- 1-yl)prop-2-en-1- one |
| 68 | 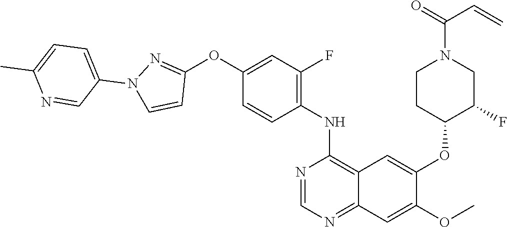 [SMILES: C=CC(=O)N1CC[C@@H](Oc2cc3c(Nc4ccc(Oc5ccn(-c6ccc(C)nc6)n5)cc4F)ncnc3cc2OC)[C@@H](F)C1] | 1-((3S,4R)-3-fluoro- 4-((4-((2-fluoro- 4-((1-(6-methylpyridin- 3-yl)-1H- pyrazol-3-yl)oxy) phenyl)amino)-7- methoxyquinazolin-6- yl)oxy)piperidin- 1-yl)prop-2-en-1- one |
| 69 | 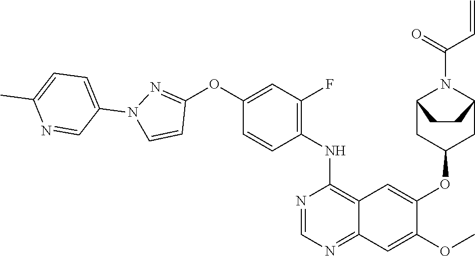 [SMILES: C=CC(=O)N1[C@@H]2CC[C@H]1C[C@@H](Oc1cc3c(Nc4ccc(Oc5ccn(-c6ccc(C)nc6)n5)cc4F)ncnc3cc1OC)C2] | 1-((1R,3r,5S)-3- ((4-((2-fluoro-4-((1- (6-methylpyridin-3- yl)-1H-pyrazol-3- yl)oxy)phenyl)amino)-7- methoxyquinazolin- 6-yl)oxy)-8- azabicyclo[3.2.1] octan-8-yl)prop-2- en-1-one |
| 70 | 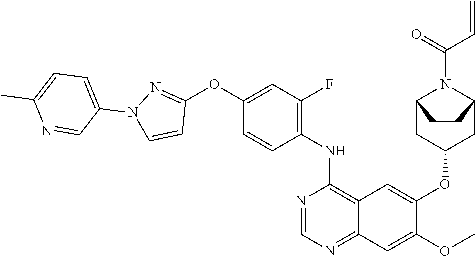 [SMILES: C=CC(=O)N1[C@@H]2CC[C@H]1C[C@H](Oc1cc3c(Nc4ccc(Oc5ccn(-c6ccc(C)nc6)n5)cc4F)ncnc3cc1OC)C2] | 1-((1R,3s,5S)-3-((4- ((2-fluoro-4-((1- (6-methylpyridin-3- yl)-1H-pyrazol-3- yl)oxy)phenyl)amino)-7- methoxyquinazolin- 6-yl)oxy)-8- azabicyclo[3.2.1] octan-8-yl)prop-2- en-1-one |
| 71 | 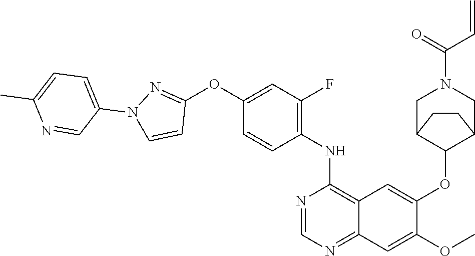 [SMILES: C=CC(=O)N1CC2CCC(C1)C2Oc1cc2c(Nc3ccc(Oc4ccn(-c5ccc(C)nc5)n4)cc3F)ncnc2cc1OC] | 1-((1R,5S,8s)-8-((4- ((2-fluoro-4-((1- (6-methylpyridin-3- yl)-1H-pyrazol-3- yl)oxy)phenyl)amino)-7- methoxyquinazolin- 6-yl)oxy)-3- azabicyclo[3.2.1] octan-3-yl)prop-2- en-1-one |
| 72 | 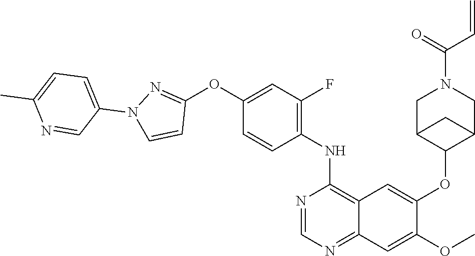 [SMILES: C=CC(=O)N1CC2CC(C1)C2Oc1cc2c(Nc3ccc(Oc4ccn(-c5ccc(C)nc5)n4)cc3F)ncnc2cc1OC] | 1-(6-((4-((2-fluoro-4-((1-(6- methylpyridin-3- yl)-1H-pyrazol-3- yl)oxy)phenyl)amino)-7- methoxyquinazolin- 6-yl)oxy)-3- azabicyclo[3.1.1] heptan-3-yl)prop-2- en-1-one |
| 73 | 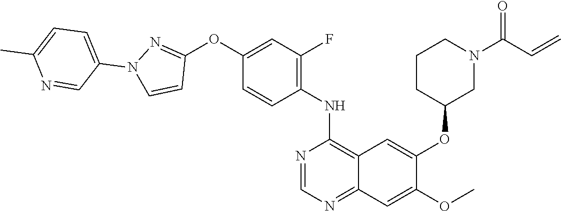 [SMILES: C=CC(=O)N1CCC[C@H](Oc2cc3c(Nc4ccc(Oc5ccn(-c6ccc(C)nc6)n5)cc4F)ncnc3cc2OC)C1] | (S)-1-(3-((4-((2- fluoro-4-((1-(6- methylpyridin-3- yl)-1H-pyrazol-3- yl)oxy)phenyl)amino)-7- methoxyquinazolin-6- yl)oxy)piperidin- 1-yl)prop-2-en-1- one |
| 74 | 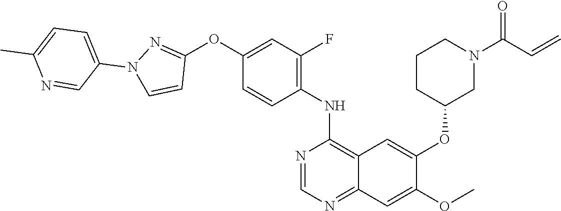 [SMILES: C=CC(=O)N1CCC[C@@H](Oc2cc3c(Nc4ccc(Oc5ccn(-c6ccc(C)nc6)n5)cc4F)ncnc3cc2OC)C1] | (R)-1-(3-((4-((2- fluoro-4-((1-(6- methylpyridin-3-yl)- 1H-pyrazol-3- yl)oxy)phenyl)amino)-7- methoxyquinazolin-6- yl)oxy)piperidin- 1-yl)prop-2-en-1- one |
| 75 | 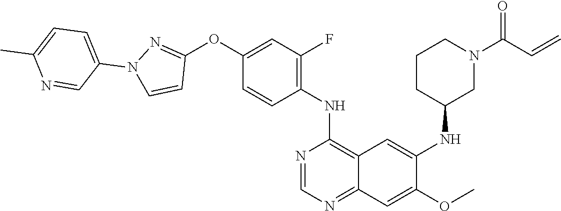 [SMILES: C=CC(=O)N1CCC[C@H](Nc2cc3c(Nc4ccc(Oc5ccn(-c6ccc(C)nc6)n5)cc4F)ncnc3cc2OC)C1] | (S)-1-(3-((4-((2- fluoro-4-((1-(6- methylpyridin-3-yl)- 1H-pyrazol-3- yl)oxy)phenyl)amino)-7- methoxyquinazolin-6- yl)amino)piperidin- 1-yl)prop-2-en-1- one |
| 76 | 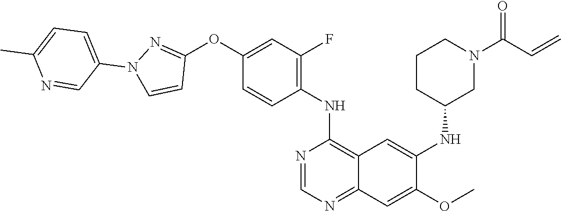 [SMILES: C=CC(=O)N1CCC[C@@H](Nc2cc3c(Nc4ccc(Oc5ccn(-c6ccc(C)nc6)n5)cc4F)ncnc3cc2OC)C1] | (R)-1-(3-((4-((2- fluoro-4-((1-(6- methylpyridin-3-yl)- 1H-pyrazol-3- yl)oxy)phenyl)amino)-7- methoxyquinazolin-6- yl)amino)piperidin- 1-yl)prop-2-en-1- one |
| 77 | 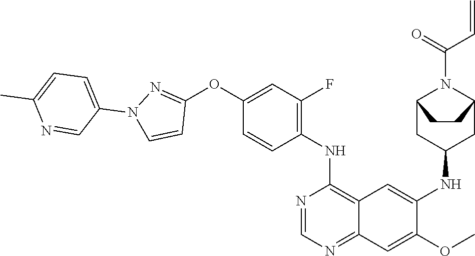 [SMILES: C=CC(=O)N1[C@@H]2CC[C@H]1C[C@@H](Nc1cc3c(Nc4ccc(Oc5ccn(-c6ccc(C)nc6)n5)cc4F)ncnc3cc1OC)C2] | 1-((1R,3r,5S)-3- ((4-((2-fluoro-4-((1- (6-methylpyridin-3- yl)-1H-pyrazol-3- yl)oxy)phenyl)amino)-7- methoxyquinazolin- 6-yl)amino)-8- azabicyclo[3.2.1] octan-8-yl)prop-2- en-1-one |
| 78 | 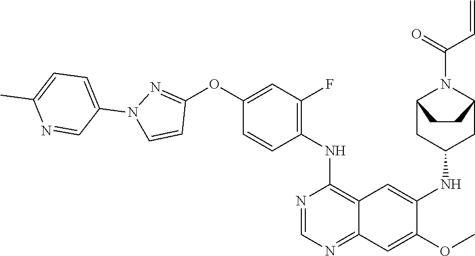 [SMILES: C=CC(=O)N1[C@@H]2CC[C@H]1C[C@H](Nc1cc3c(Nc4ccc(Oc5ccn(-c6ccc(C)nc6)n5)cc4F)ncnc3cc1OC)C2] | 1-((1R,3s,5S)-3- ((4-((2-fluoro-4-((1- (6-methylpyridin-3- yl)-1H-pyrazol-3- yl)oxy)phenyl)amino)-7- methoxyquinazolin- 6-yl)amino)-8- azabicyclo[3.2.1] octan-8-yl)prop-2- en-1-one |
| 79 | 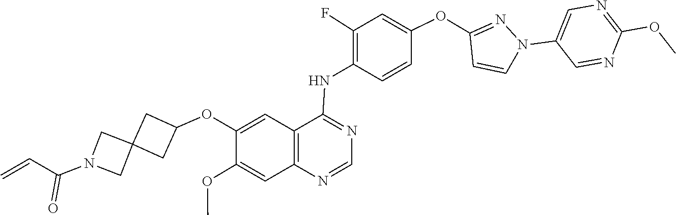 [SMILES: C=CC(=O)N1CC2(CC(Oc3cc4c(Nc5ccc(Oc6ccn(-c7cnc(OC)nc7)n6)cc5F)ncnc4cc3OC)C2)C1] | 1-(6-((4-((2-fluoro-4-((1-(2- methoxypyrimidin-5- yl)-1H-pyrazol- 3-yl)oxy)phenyl)amino)-7- methoxyquinazolin- 6-yl)oxy)-2- azaspiro[3.3]heptan- 2-yl)prop-2-en- 1-one |
| 80 | 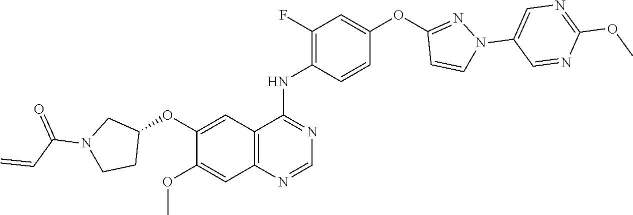 [SMILES: C=CC(=O)N1CC[C@@H](Oc2cc3c(Nc4ccc(Oc5ccn(-c6cnc(OC)nc6)n5)cc4F)ncnc3cc2OC)C1] | (R)-1-(3-((4-((2- fluoro-4-((1-(2- methoxypyrimidin- 5-yl)-1H-pyrazol- 3-yl)oxy)phenyl)amino)-7- methoxyquinazolin-6- yl)oxy)pyrrolidin- 1-yl)prop-2-en-1- one |
| 81 | 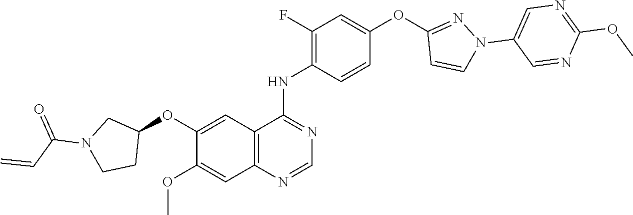 [SMILES: C=CC(=O)N1CC[C@H](Oc2cc3c(Nc4ccc(Oc5ccn(-c6cnc(OC)nc6)n5)cc4F)ncnc3cc2OC)C1] | (S)-1-(3-((4-((2- fluoro-4-((1-(2- methoxypyrimidin- 5-yl)-1H-pyrazol- 3-yl)oxy)phenyl)amino)-7- methoxyquinazolin-6- yl)oxy)pyrrolidin- 1-yl)prop-2-en-1- one |
| 82 | 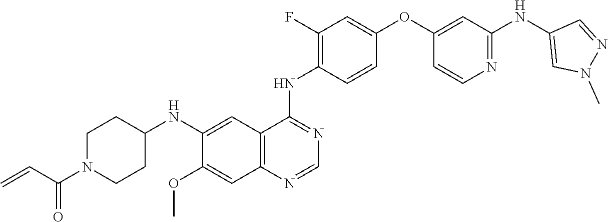 [SMILES: C=CC(=O)N1CCC(Nc2cc3c(Nc4ccc(Oc5ccnc(Nc6cnn(C)c6)c5)cc4F)ncnc3cc2OC)CC1] | 1-(4-((4-((2-fluoro- 4-((2-((1-methyl- 1H-pyrazol-4-yl) amino)pyridin-4- yl)oxy)phenyl)amino)-7- methoxyquinazolin-6- yl)amino)piperidin- 1-yl)prop-2-en-1- one |
| 83 | 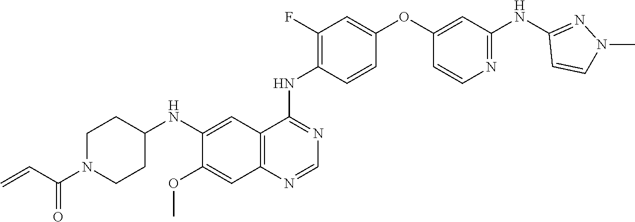 [SMILES: C=CC(=O)N1CCC(Nc2cc3c(Nc4ccc(Oc5ccnc(Nc6ccn(C)n6)c5)cc4F)ncnc3cc2OC)CC1] | 1-(4-((4-((2-fluoro- 4-((2-((1-methyl- 1H-pyrazol-3-yl) amino)pyridin-4- yl)oxy)phenyl)amino)-7- methoxyquinazolin-6- yl)amino)piperidin- 1-yl)prop-2-en-1- one |
| 84 | 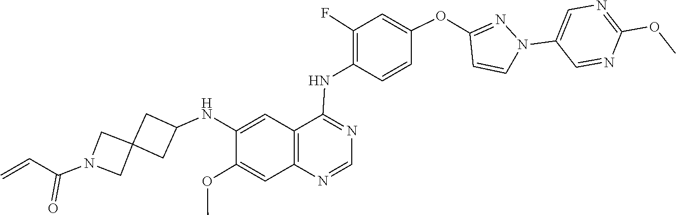 [SMILES: C=CC(=O)N1CC2(CC(Nc3cc4c(Nc5ccc(Oc6ccn(-c7cnc(OC)nc7)n6)cc5F)ncnc4cc3OC)C2)C1] | 1-(6-((4-((2-fluoro-4-((1-(2- methoxypyrimidin- 5-yl)-1H-pyrazol- 3-yl)oxy)phenyl)amino)-7- methoxyquinazolin- 6-yl)amino)-2- azaspiro[3.3]heptan- 2-yl)prop-2-en- 1-one |
| 85 | 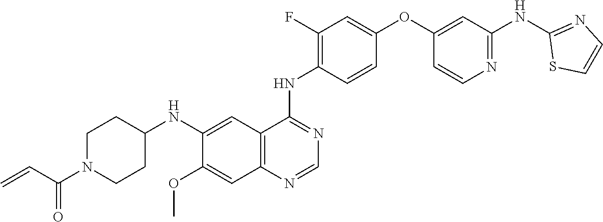 [SMILES: C=CC(=O)N1CCC(Nc2cc3c(Nc4ccc(Oc5ccnc(Nc6nccs6)c5)cc4F)ncnc3cc2OC)CC1] | 1-(4-((4-((2-fluoro- 4-((2-(thiazol-2- ylamino)pyridin-4- yl)oxy)phenyl)amino)-7- methoxyquinazolin-6- yl)amino)piperidin- 1-yl)prop-2-en-1- one |
| 86 | 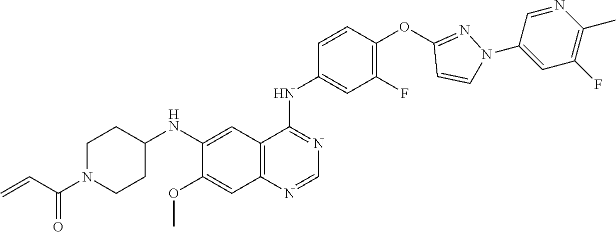 [SMILES: C=CC(=O)N1CCC(Nc2cc3c(Nc4ccc(Oc5ccn(-c6cnc(C)c(F)c6)n5)c(F)c4)ncnc3cc2OC)CC1] | 1-(4-((4-((3-fluoro- 4-((1-(5-fluoro-6- methylpyridin-3-yl)- 1H-pyrazol-3- yl)oxy)phenyl)amino)-7- methoxyquinazolin-6- yl)amino)piperidin- 1-yl)prop-2-en-1- one |
| 87 | 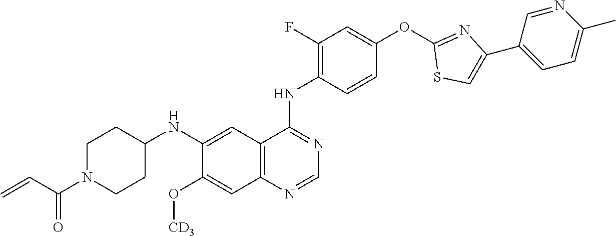 [SMILES: [2H]C([2H])([2H])Oc1cc2ncnc(Nc3ccc(Oc4nc(-c5ccc(C)nc5)cs4)cc3F)c2cc1NC1CCN(C(=O)C=C)CC1] | 1-(4-((4-((2-fluoro-4-((4-(6- methylpyridin-3-yl)thiazol-2- yl)oxy)phenyl)amino)- 7-(methoxy- d3)quinazolin-6-yl) amino)piperidin- 1-yl)prop-2-en-1-one |
| 88 | 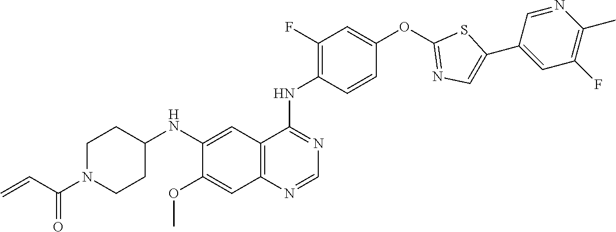 [SMILES: C=CC(=O)N1CCC(Nc2cc3c(Nc4ccc(Oc5ncc(-c6cnc(C)c(F)c6)s5)cc4F)ncnc3cc2OC)CC1] | 1-(4-((4-((2-fluoro- 4-((5-(5-fluoro-6- methylpyridin-3-yl)thiazol-2- yl)oxy)phenyl)amino)-7- methoxyquinazolin-6- yl)amino)piperidin- 1-yl)prop-2-en-1- one |
| 89 | 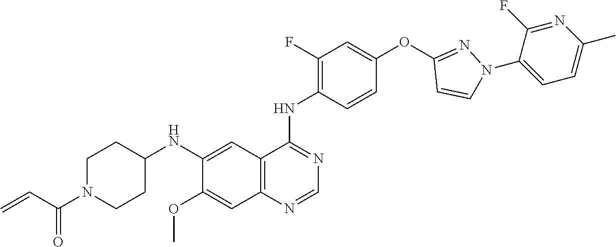 [SMILES: C=CC(=O)N1CCC(Nc2cc3c(Nc4ccc(Oc5ccn(-c6ccc(C)nc6F)n5)cc4F)ncnc3cc2OC)CC1] | 1-(4-((4-((2-fluoro- 4-((1-(2-fluoro-6- methylpyridin-3-yl)- 1H-pyrazol-3- yl)oxy)phenyl)amino)-7- methoxyquinazolin-6- yl)amino)piperidin- 1-yl)prop-2-en-1- one |
| 90 | 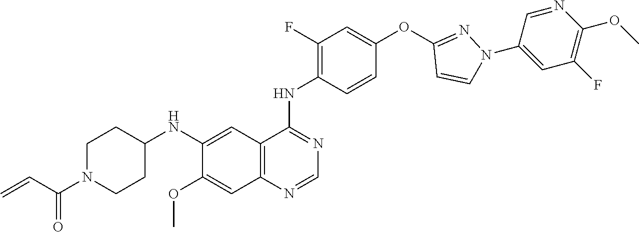 [SMILES: C=CC(=O)N1CCC(Nc2cc3c(Nc4ccc(Oc5ccn(-c6cnc(OC)c(F)c6)n5)cc4F)ncnc3cc2OC)CC1] | 1-(4-((4-((2-fluoro- 4-((1-(5-fluoro-6- methoxypyridin-3-yl)- 1H-pyrazol-3- yl)oxy)phenyl)amino)-7- methoxyquinazolin-6- yl)amino)piperidin- 1-yl)prop-2-en-1- one |
| 91 | 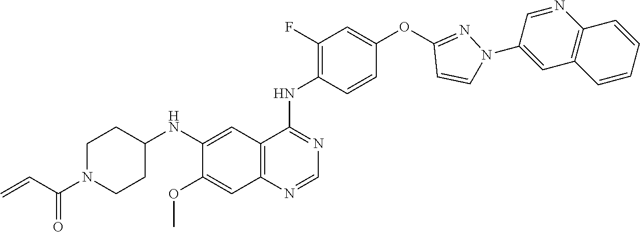 [SMILES: C=CC(=O)N1CCC(Nc2cc3c(Nc4ccc(Oc5ccn(-c6cnc7ccccc7c6)n5)cc4F)ncnc3cc2OC)CC1] | 1-(4-((4-((2-fluoro- 4-((1-(quinolin-3- yl)-1H-pyrazol-3- yl)oxy)phenyl)amino)-7- methoxyquinazolin-6- yl)amino)piperidin- 1-yl)prop-2-en-1- one |
| 92 | 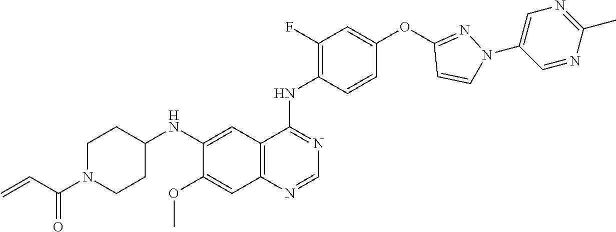 [SMILES: C=CC(=O)N1CCC(Nc2cc3c(Nc4ccc(Oc5ccn(-c6cnc(C)nc6)n5)cc4F)ncnc3cc2OC)CC1] | 1-(4-((4-((2-fluoro-4-((1-(2- methylpyrimidin-5- yl)-1H-pyrazol-3- yl)oxy)phenyl)amino)-7- methoxyquinazolin-6- yl)amino)piperidin- 1-yl)prop-2-en-1- one |
| 93 | 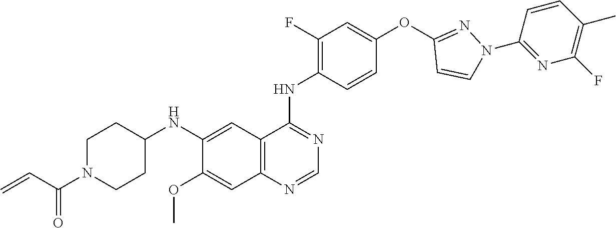 [SMILES: C=CC(=O)N1CCC(Nc2cc3c(Nc4ccc(Oc5ccn(-c6ccc(C)c(F)n6)n5)cc4F)ncnc3cc2OC)CC1] | 1-(4-((4-((2-fluoro- 4-((1-(6-fluoro-5- methylpyridin-2- yl)-1H-pyrazol-3- yl)oxy)phenyl)amino)-7- methoxyquinazolin-6- yl)amino)piperidin- 1-yl)prop-2-en-1- one |
| 94 | 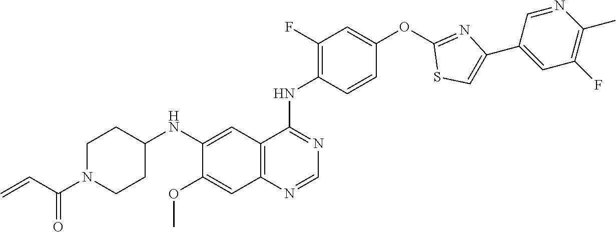 [SMILES: C=CC(=O)N1CCC(Nc2cc3c(Nc4ccc(Oc5nc(-c6cnc(C)c(F)c6)cs5)cc4F)ncnc3cc2OC)CC1] | 1-(4-((4-((2-fluoro- 4-((4-(5-fluoro-6- methylpyridin-3-yl)thiazol-2- yl)oxy)phenyl)amino)-7- methoxyquinazolin-6- yl)amino)piperidin- 1-yl)prop-2-en-1- one |
| 95 | 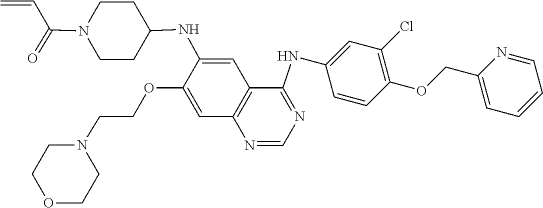 [SMILES: C=CC(=O)N1CCC(Nc2cc3c(Nc4ccc(OCc5ccccn5)c(Cl)c4)ncnc3cc2OCCN2CCOCC2)CC1] | 1-(4-((4-((3-chloro- 4-(pyridin-2- ylmethoxy)phenyl) amino)-7-(2- morpholinoethoxy) quinazolin-6- yl)amino)piperidin- 1-yl)prop-2-en-1- one |
| 96 | 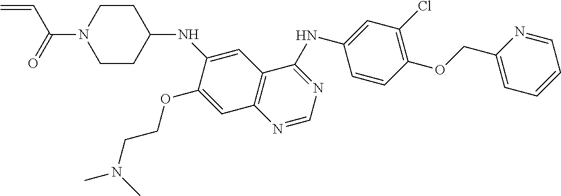 [SMILES: C=CC(=O)N1CCC(Nc2cc3c(Nc4ccc(OCc5ccccn5)c(Cl)c4)ncnc3cc2OCCN(C)C)CC1] | 1-(4-((4-((3-chloro- 4-(pyridin-2- ylmethoxy)phenyl) amino)-7-(2- (dimethylamino) ethoxy)quinazolin-6- yl)amino)piperidin- 1-yl)prop-2-en-1- one |
| 97 | 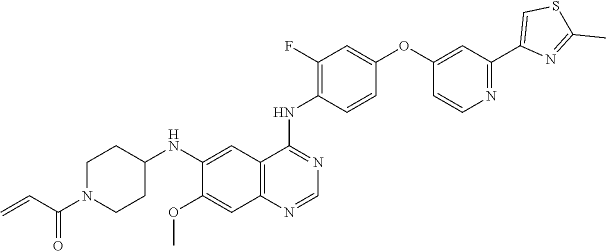 [SMILES: C=CC(=O)N1CCC(Nc2cc3c(Nc4ccc(Oc5ccnc(-c6csc(C)n6)c5)cc4F)ncnc3cc2OC)CC1] | 1-(4-((4-((2-fluoro-4-((2-(2- methylthiazol-4-yl)pyridin-4- yl)oxy)phenyl)amino)-7- methoxyquinazolin-6- yl)amino)piperidin- 1-yl)prop-2-en-1- one |
| 98 | 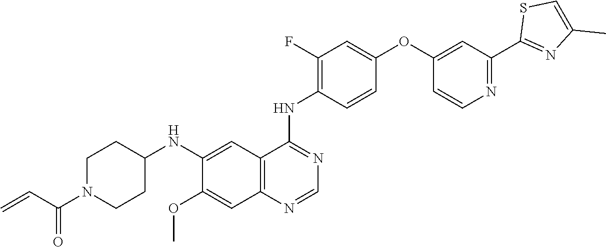 [SMILES: C=CC(=O)N1CCC(Nc2cc3c(Nc4ccc(Oc5ccnc(-c6nc(C)cs6)c5)cc4F)ncnc3cc2OC)CC1] | 1-(4-((4-((2-fluoro-4-((2-(4- methylthiazol-2- yl)pyridin-4- yl)oxy)phenyl)amino)-7- methoxyquinazolin-6- yl)amino)piperidin- 1-yl)prop-2-en-1- one |
| 99 | 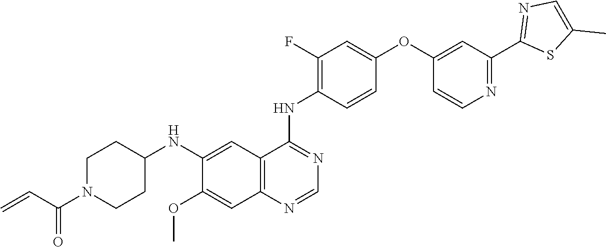 [SMILES: C=CC(=O)N1CCC(Nc2cc3c(Nc4ccc(Oc5ccnc(-c6ncc(C)s6)c5)cc4F)ncnc3cc2OC)CC1] | 1-(4-((4-((2-fluoro-4-((2-(5- methylthiazol-2- yl)pyridin-4- yl)oxy)phenyl)amino)-7- methoxyquinazolin-6- yl)amino)piperidin- 1-yl)prop-2-en-1- one |
| 100 | 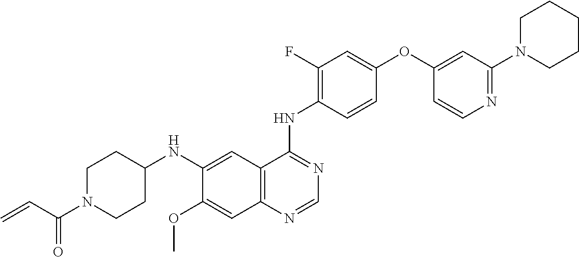 [SMILES: C=CC(=O)N1CCC(Nc2cc3c(Nc4ccc(Oc5ccnc(N6CCCCC6)c5)cc4F)ncnc3cc2OC)CC1] | 1-(4-((4-((2-fluoro- 4-((2-(piperidin-1- yl)pyridin-4-yl)oxy) phenyl)amino)-7- methoxyquinazolin-6- yl)amino)piperidin- 1-yl)prop-2-en-1- one |
| 101 | 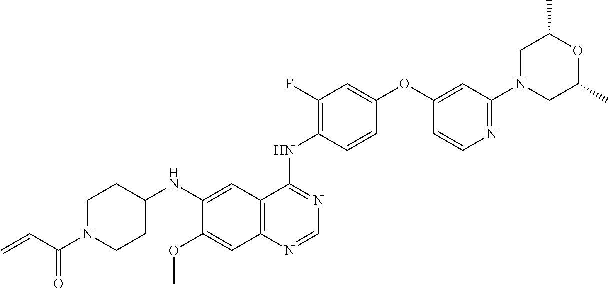 [SMILES: C=CC(=O)N1CCC(Nc2cc3c(Nc4ccc(Oc5ccnc(N6C[C@@H](C)O[C@@H](C)C6)c5)cc4F)ncnc3cc2OC)CC1] | 1-(4-((4-((4-((2-((2R,6S)-2,6- dimethylmorpholino) pyridin-4- yl)oxy)-2-fluorophenyl) amino)-7- methoxyquinazolin-6- yl)amino)piperidin- 1-yl)prop-2-en-1- one |
| 102 | 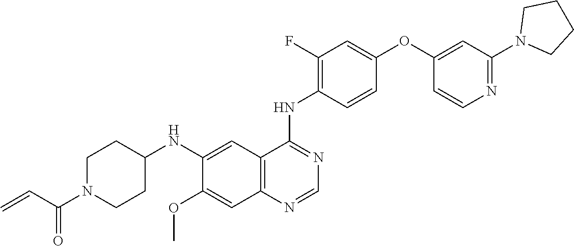 [SMILES: C=CC(=O)N1CCC(Nc2cc3c(Nc4ccc(Oc5ccnc(N6CCCC6)c5)cc4F)ncnc3cc2OC)CC1] | 1-(4-((4-((2-fluoro- 4-((2-(pyrrolidin-1- yl)pyridin-4-yl)oxy) phenyl)amino)-7- methoxyquinazolin-6- yl)amino)piperidin- 1-yl)prop-2-en-1- one |
| 103 | 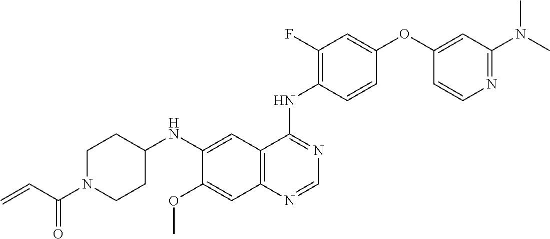 [SMILES: C=CC(=O)N1CCC(Nc2cc3c(Nc4ccc(Oc5ccnc(N(C)C)c5)cc4F)ncnc3cc2OC)CC1] | 1-(4-((4-((4-((2- (dimethylamino) pyridin-4-yl)oxy)-2- fluorophenyl)amino)-7- methoxyquinazolin-6- yl)amino)piperidin- 1-yl)prop-2-en-1- one |
| 104 | 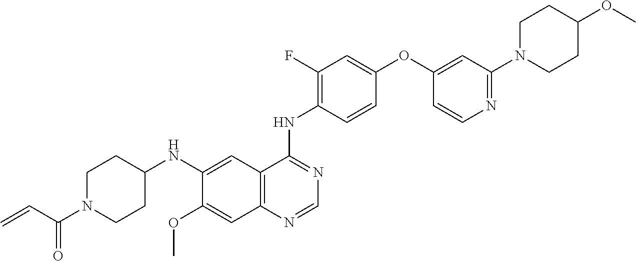 [SMILES: C=CC(=O)N1CCC(Nc2cc3c(Nc4ccc(Oc5ccnc(N6CCC(OC)CC6)c5)cc4F)ncnc3cc2OC)CC1] | 1-(4-((4-((2-fluoro-4-((2-(4- methoxypiperidin- 1-yl)pyridin-4- yl)oxy)phenyl)amino)-7- methoxyquinazolin-6- yl)amino)piperidin- 1-yl)prop-2-en-1- one |
| 105 | 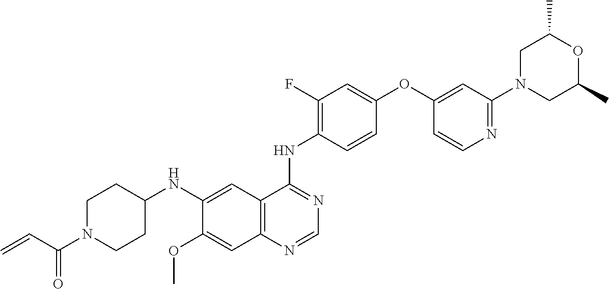 [SMILES: C=CC(=O)N1CCC(Nc2cc3c(Nc4ccc(Oc5ccnc(N6C[C@H](C)O[C@@H](C)C6)c5)cc4F)ncnc3cc2OC)CC1] | 1-(4-((4-((4-((2-((2S,6S)-2,6- dimethylmorpholino) pyridin-4- yl)oxy)-2-fluorophenyl) amino)-7- methoxyquinazolin-6- yl)amino)piperidin- 1-yl)prop-2-en-1- one |
| 106 | 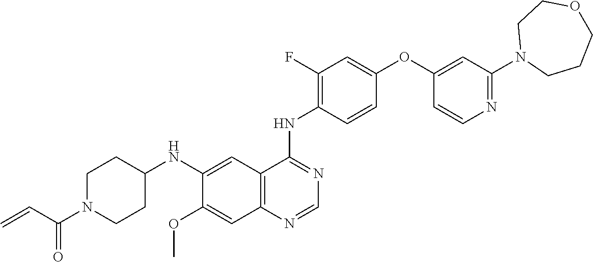 [SMILES: C=CC(=O)N1CCC(Nc2cc3c(Nc4ccc(Oc5ccnc(N6CCCOCC6)c5)cc4F)ncnc3cc2OC)CC1] | 1-(4-((4-((4-((2- (1,4-oxazepan-4- yl)pyridin-4-yl)oxy)-2- fluorophenyl)amino)-7- methoxyquinazolin-6- yl)amino)piperidin- 1-yl)prop-2-en-1- one |
| 107 | 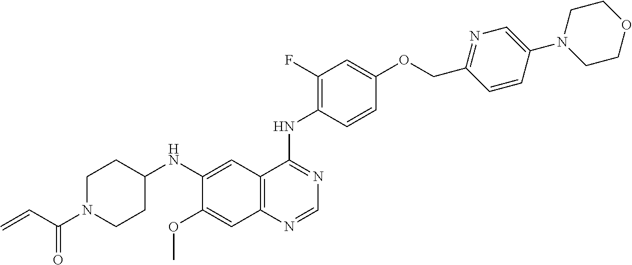 [SMILES: C=CC(=O)N1CCC(Nc2cc3c(Nc4ccc(OCc5ccc(N6CCOCC6)cn5)cc4F)ncnc3cc2OC)CC1] | 1-(4-((4-((2-fluoro-4-((5- morpholinopyridin-2- yl)methoxy)phenyl) amino)-7- methoxyquinazolin-6- yl)amino)piperidin- 1-yl)prop-2-en-1- one |
| 108 | 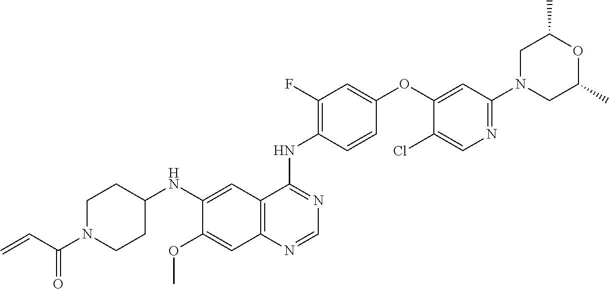 [SMILES: C=CC(=O)N1CCC(Nc2cc3c(Nc4ccc(Oc5cc(N6C[C@@H](C)O[C@@H](C)C6)ncc5Cl)cc4F)ncnc3cc2OC)CC1] | 1-(4-((4-((4-((5- chloro-2-((2R,6S)- 2,6-dimethylmorpholino) pyridin-4- yl)oxy)-2-fluorophenyl) amino)-7- methoxyquinazolin-6- yl)amino)piperidin- 1-yl)prop-2-en-1- one |
| 109 | 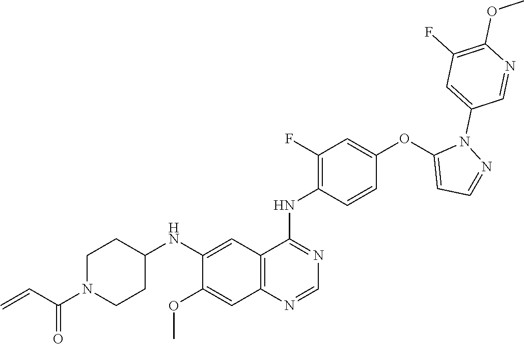 [SMILES: C=CC(=O)N1CCC(Nc2cc3c(Nc4ccc(Oc5ccnn5-c5cnc(OC)c(F)c5)cc4F)ncnc3cc2OC)CC1] | 1-(4-((4-((2-fluoro- 4-((1-(5-fluoro-6- methoxypyridin-3- yl)-1H-pyrazol-5- yl)oxy)phenyl)amino)-7- methoxyquinazolin-6- yl)amino)piperidin- 1-yl)prop-2-en-1- one |
| 110 | 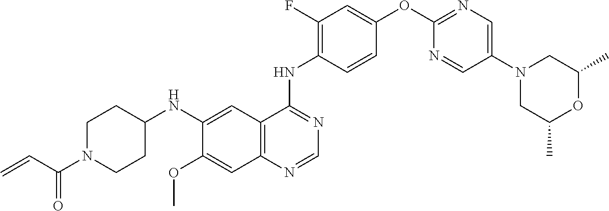 [SMILES: C=CC(=O)N1CCC(Nc2cc3c(Nc4ccc(Oc5ncc(N6C[C@@H](C)O[C@@H](C)C6)cn5)cc4F)ncnc3cc2OC)CC1] | 1-(4-((4-((4-((5-((2R,6S)-2,6- dimethylmorpholino) pyrimidin-2- yl)oxy)-2-fluorophenyl) amino)-7- methoxyquinazolin-6- yl)amino)piperidin- 1-yl)prop-2-en-1- one |
| 111 | 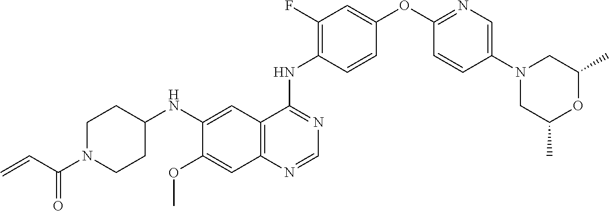 [SMILES: C=CC(=O)N1CCC(Nc2cc3c(Nc4ccc(Oc5ccc(N6C[C@@H](C)O[C@@H](C)C6)cn5)cc4F)ncnc3cc2OC)CC1] | 1-(4-((4-((4-((5-((2R,6S)-2,6- dimethylmorpholino) pyridin-2- yl)oxy)-2-fluorophenyl) amino)-7- methoxyquinazolin-6- yl)amino)piperidin- 1-yl)prop-2-en-1- one |
| 112 | 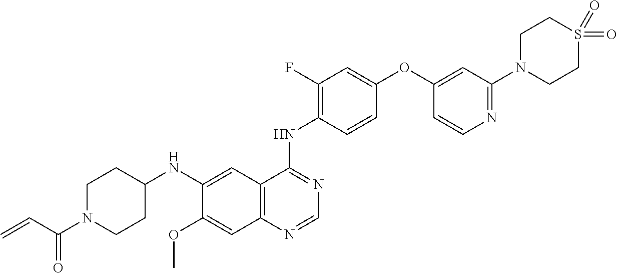 [SMILES: C=CC(=O)N1CCC(Nc2cc3c(Nc4ccc(Oc5ccnc(N6CCS(=O)(=O)CC6)c5)cc4F)ncnc3cc2OC)CC1] | 1-(4-((4-((4-((2-(1,1- dioxidothiomorpholino) pyridin-4- yl)oxy)-2-fluorophenyl) amino)-7- methoxyquinazolin-6- yl)amino)piperidin- 1-yl)prop-2-en-1- one |
| 113 | 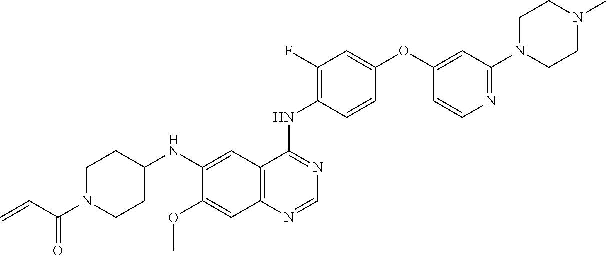 [SMILES: C=CC(=O)N1CCC(Nc2cc3c(Nc4ccc(Oc5ccnc(N6CCN(C)CC6)c5)cc4F)ncnc3cc2OC)CC1] | 1-(4-((4-((2-fluoro-4-((2-(4- methylpiperazin-1- yl)pyridin-4- yl)oxy)phenyl)amino)-7- methoxyquinazolin-6- yl)amino)piperidin- 1-yl)prop-2-en-1- one |
| 114 | 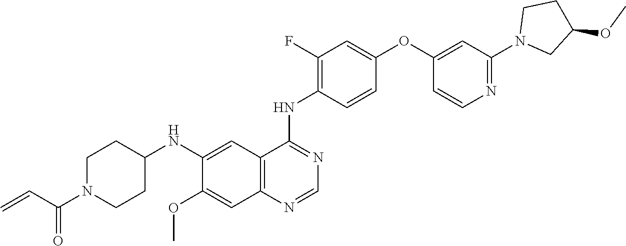 [SMILES: C=CC(=O)N1CCC(Nc2cc3c(Nc4ccc(Oc5ccnc(N6CC[C@@H](OC)C6)c5)cc4F)ncnc3cc2OC)CC1] | (R)-1-(4-((4-((2- fluoro-4-((2-(3- methoxypyrrolidin-1- yl)pyridin-4- yl)oxy)phenyl)amino)-7- methoxyquinazolin-6- yl)amino)piperidin- 1-yl)prop-2-en-1- one |
| 115 | 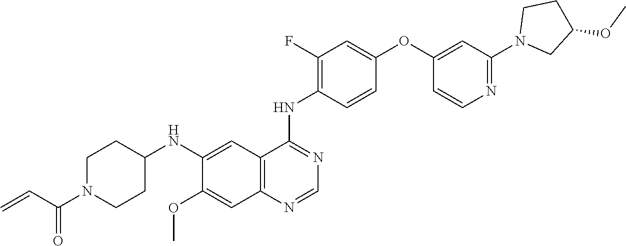 [SMILES: C=CC(=O)N1CCC(Nc2cc3c(Nc4ccc(Oc5ccnc(N6CC[C@H](OC)C6)c5)cc4F)ncnc3cc2OC)CC1] | (S)-1-(4-((4-((2- fluoro-4-((2-(3- methoxypyrrolidin-1- yl)pyridin-4- yl)oxy)phenyl)amino)-7- methoxyquinazolin-6- yl)amino)piperidin- 1-yl)prop-2-en-1- one |
| 116 | 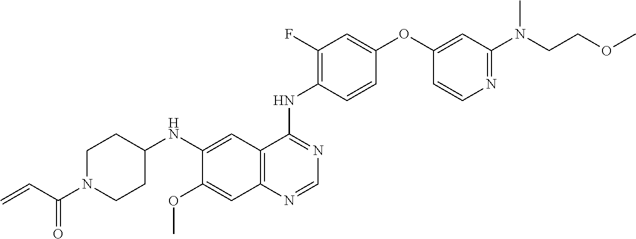 [SMILES: C=CC(=O)N1CCC(Nc2cc3c(Nc4ccc(Oc5ccnc(N(C)CCOC)c5)cc4F)ncnc3cc2OC)CC1] | 1-(4-((4-((2-fluoro-4-((2-((2- methoxyethyl)(methyl) amino)pyridin- 4-yl)oxy)phenyl)amino)-7- methoxyquinazolin-6- yl)amino)piperidin- 1-yl)prop-2-en-1- one |
| 117 | 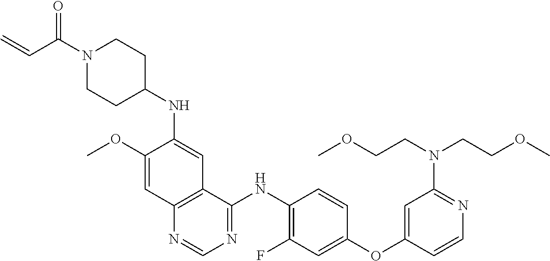 [SMILES: C=CC(=O)N1CCC(Nc2cc3c(Nc4ccc(Oc5ccnc(N(CCOC)CCOC)c5)cc4F)ncnc3cc2OC)CC1] | 1-(4-((4-((4-((2-(bis(2- methoxyethyl) amino)pyridin-4- yl)oxy)-2- fluorophenyl)amino)-7- methoxyquinazolin-6- yl)amino)piperidin- 1-yl)prop-2-en-1- one |
| 118 | 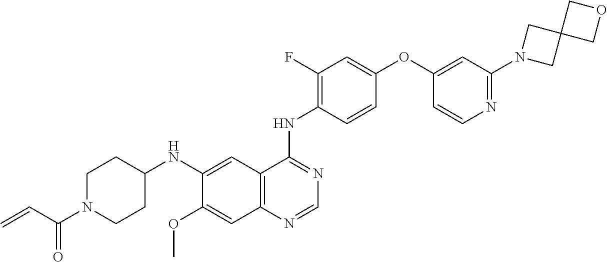 [SMILES: C=CC(=O)N1CCC(Nc2cc3c(Nc4ccc(Oc5ccnc(N6CC7(COC7)C6)c5)cc4F)ncnc3cc2OC)CC1] | 1-(4-((4-((4-((2-(2-oxa-6- azaspiro[3.3]heptan- 6-yl)pyridin-4- yl)oxy)-2- fluorophenyl)amino)-7- methoxyquinazolin-6- yl)amino)piperidin- 1-yl)prop-2-en-1- one |
| 119 | 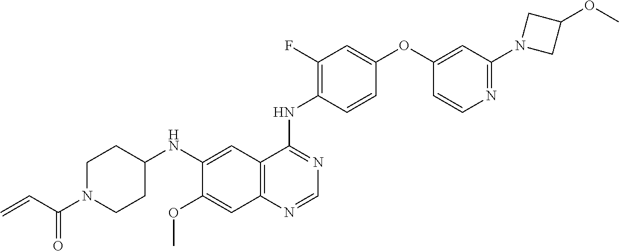 [SMILES: C=CC(=O)N1CCC(Nc2cc3c(Nc4ccc(Oc5ccnc(N6CC(OC)C6)c5)cc4F)ncnc3cc2OC)CC1] | 1-(4-((4-((2-fluoro-4-((2-(3- methoxyazetidin- 1-yl)pyridin-4- yl)oxy)phenyl)amino)-7- methoxyquinazolin-6- yl)amino)piperidin- 1-yl)prop-2-en-1- one |
| 120 | 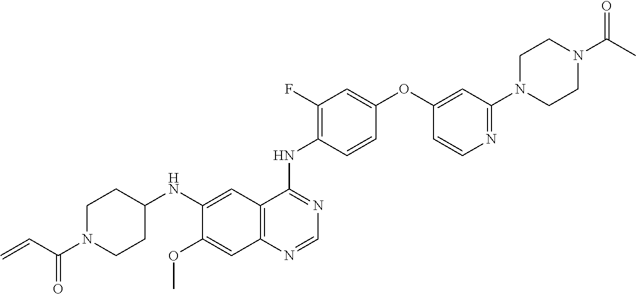 [SMILES: C=CC(=O)N1CCC(Nc2cc3c(Nc4ccc(Oc5ccnc(N6CCN(C(C)=O)CC6)c5)cc4F)ncnc3cc2OC)CC1] | 1-(4-((4-((4-((2-(4- acetylpiperazin-1- yl)pyridin-4-yl)oxy)-2- fluorophenyl)amino)-7- methoxyquinazolin-6- yl)amino)piperidin- 1-yl)prop-2-en-1- one |
| 121 | 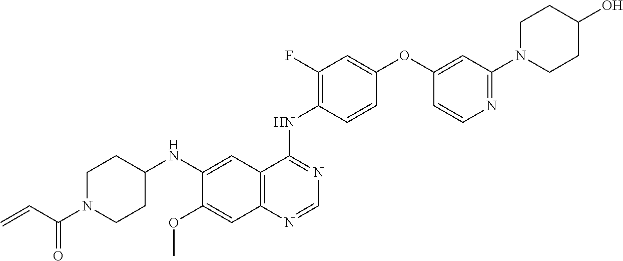 [SMILES: C=CC(=O)N1CCC(Nc2cc3c(Nc4ccc(Oc5ccnc(N6CCC(O)CC6)c5)cc4F)ncnc3cc2OC)CC1] | 1-(4-((4-((2-fluoro-4-((2-(4- hydroxypiperidin- 1-yl)pyridin-4- yl)oxy)phenyl)amino)-7- methoxyquinazolin-6- yl)amino)piperidin- 1-yl)prop-2-en-1- one |
| 122 | 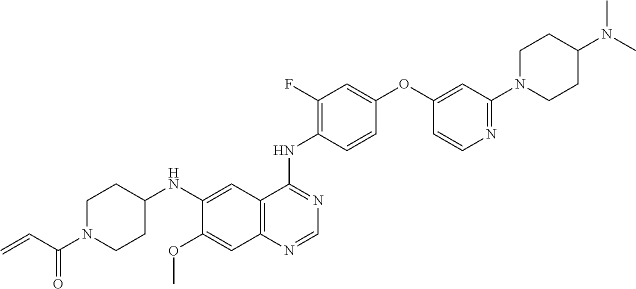 [SMILES: C=CC(=O)N1CCC(Nc2cc3c(Nc4ccc(Oc5ccnc(N6CCC(N(C)C)CC6)c5)cc4F)ncnc3cc2OC)CC1] | 1-(4-((4-((4-((2-(4- (dimethylamino)piperidin-1- yl)pyridin-4-yl)oxy)-2- fluorophenyl)amino)-7- methoxyquinazolin-6- yl)amino)piperidin- 1-yl)prop-2-en-1- one |
| 123 | 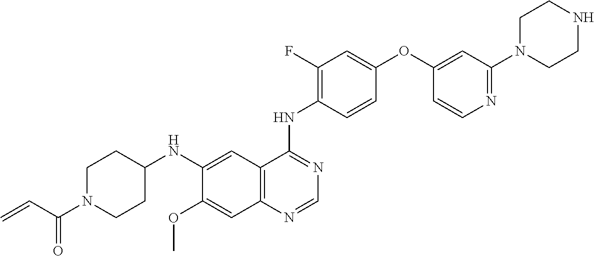 [SMILES: C=CC(=O)N1CCC(Nc2cc3c(Nc4ccc(Oc5ccnc(N6CCNCC6)c5)cc4F)ncnc3cc2OC)CC1] | 1-(4-((4-((2-fluoro- 4-((2-(piperazin-1- yl)pyridin-4-yl)oxy) phenyl)amino)-7- methoxyquinazolin-6- yl)amino)piperidin- 1-yl)prop-2-en-1- one |
| 124 | 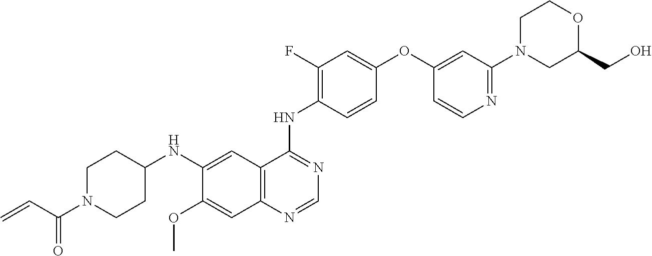 [SMILES: C=CC(=O)N1CCC(Nc2cc3c(Nc4ccc(Oc5ccnc(N6CCO[C@@H](CO)C6)c5)cc4F)ncnc3cc2OC)CC1] | (R)-1-(4-((4-((2- fluoro-4-((2-(2- (hydroxymethyl) morpholino)pyridin- 4-yl)oxy)phenyl)amino)-7- methoxyquinazolin-6- yl)amino)piperidin- 1-yl)prop-2-en-1- one |
| 125 | 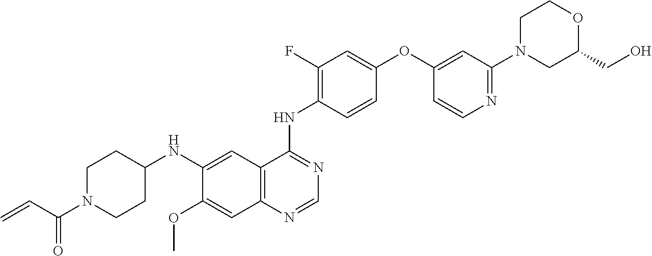 [SMILES: C=CC(=O)N1CCC(Nc2cc3c(Nc4ccc(Oc5ccnc(N6CCO[C@H](CO)C6)c5)cc4F)ncnc3cc2OC)CC1] | (S)-1-(4-((4-((2- fluoro-4-((2-(2- (hydroxymethyl) morpholino)pyridin- 4-yl)oxy)phenyl)amino)-7- methoxyquinazolin-6- yl)amino)piperidin- 1-yl)prop-2-en-1- one |
| 126 | 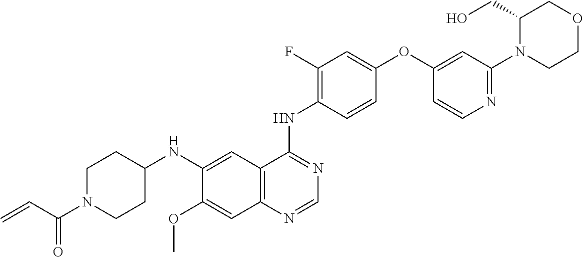 [SMILES: C=CC(=O)N1CCC(Nc2cc3c(Nc4ccc(Oc5ccnc(N6CCOC[C@H]6CO)c5)cc4F)ncnc3cc2OC)CC1] | (R)-1-(4-((4-((2- fluoro-4-((2-(3- (hydroxymethyl) morpholino)pyridin- 4-yl)oxy)phenyl)amino)-7- methoxyquinazolin-6- yl)amino)piperidin- 1-yl)prop-2-en-1- one |
| 127 | 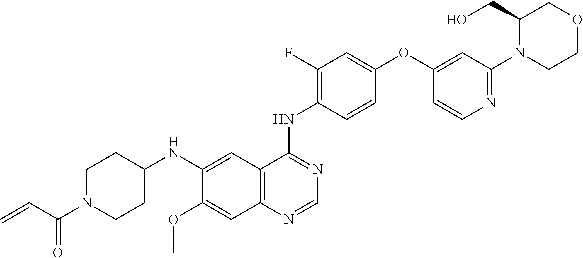 [SMILES: C=CC(=O)N1CCC(Nc2cc3c(Nc4ccc(Oc5ccnc(N6CCOC[C@@H]6CO)c5)cc4F)ncnc3cc2OC)CC1] | (S)-1-(4-((4-((2- fluoro-4-((2-(3- (hydroxymethyl) morpholino)pyridin- 4-yl)oxy)phenyl)amino)-7- methoxyquinazolin-6- yl)amino)piperidin- 1-yl)prop-2-en-1- one |
| 128 | 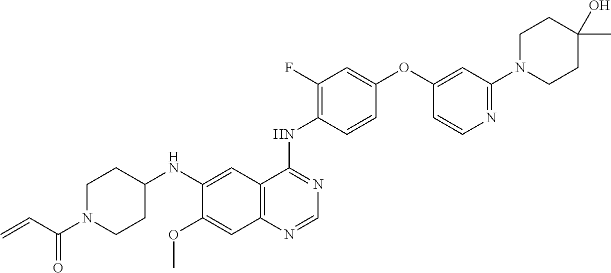 [SMILES: C=CC(=O)N1CCC(Nc2cc3c(Nc4ccc(Oc5ccnc(N6CCC(C)(O)CC6)c5)cc4F)ncnc3cc2OC)CC1] | 1-(4-((4-((2-fluoro- 4-((2-(4-hydroxy- 4-methylpiperidin- 1-yl)pyridin-4- yl)oxy)phenyl)amino)-7- methoxyquinazolin-6- yl)amino)piperidin- 1-yl)prop-2-en-1- one |
| 129 | 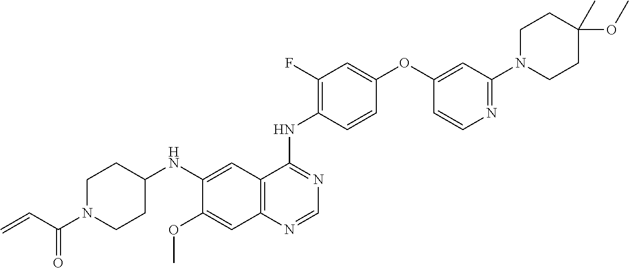 [SMILES: C=CC(=O)N1CCC(Nc2cc3c(Nc4ccc(Oc5ccnc(N6CCC(C)(OC)CC6)c5)cc4F)ncnc3cc2OC)CC1] | 1-(4-((4-((2-fluoro- 4-((2-(4-methoxy- 4-methylpiperidin-1- yl)pyridin-4- yl)oxy)phenyl)amino)-7- methoxyquinazolin-6- yl)amino)piperidin- 1-yl)prop-2-en-1- one |
| 130 | 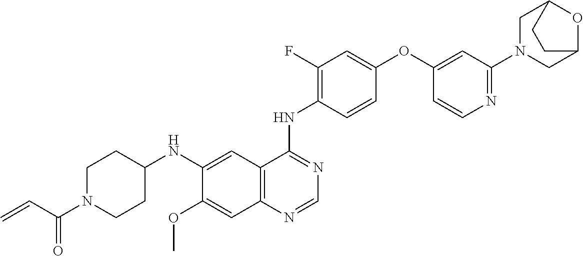 [SMILES: C=CC(=O)N1CCC(Nc2cc3c(Nc4ccc(Oc5ccnc(N6CC7CCC(C6)O7)c5)cc4F)ncnc3cc2OC)CC1] | 1-(4-((4-((4-((2-(8-oxa-3- azabicyclo[3.2.1] octan-3-yl)pyridin- 4-yl)oxy)-2-fluorophenyl) amino)-7- methoxyquinazolin-6- yl)amino)piperidin- 1-yl)prop-2-en-1- one |
| 131 | 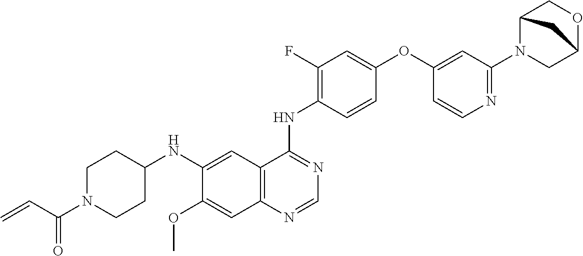 [SMILES: C=CC(=O)N1CCC(Nc2cc3c(Nc4ccc(Oc5ccnc(N6C[C@@H]7C[C@H]6CO7)c5)cc4F)ncnc3cc2OC)CC1] | 1-(4-((4-((4-((2- ((1S,4S)-2-oxa-5- azabicyclo[2.2.1] heptan-5-yl)pyridin- 4-yl)oxy)-2-fluorophenyl) amino)-7- methoxyquinazolin-6- yl)amino)piperidin- 1-yl)prop-2-en-1- one |
| 132 | 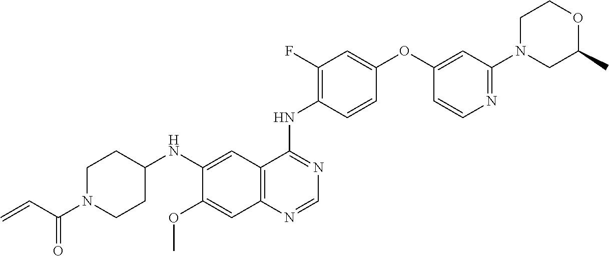 [SMILES: C=CC(=O)N1CCC(Nc2cc3c(Nc4ccc(Oc5ccnc(N6CCO[C@@H](C)C6)c5)cc4F)ncnc3cc2OC)CC1] | (S)-1-(4-((4-((2- fluoro-4-((2-(2- methylmorpholino)pyridin-4- yl)oxy)phenyl)amino)-7- methoxyquinazolin-6- yl)amino)piperidin- 1-yl)prop-2-en-1- one |
| 133 | 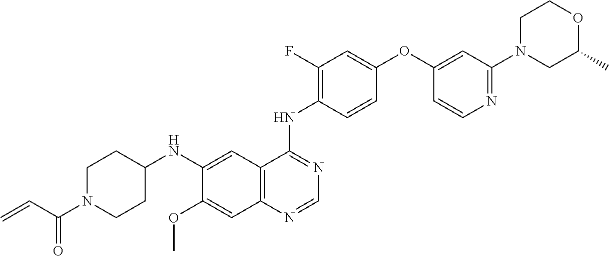 [SMILES: C=CC(=O)N1CCC(Nc2cc3c(Nc4ccc(Oc5ccnc(N6CCO[C@H](C)C6)c5)cc4F)ncnc3cc2OC)CC1] | (R)-1-(4-((4-((2- fluoro-4-((2-(2- methylmorpholino)pyridin-4- yl)oxy)phenyl)amino)-7- methoxyquinazolin-6- yl)amino)piperidin- 1-yl)prop-2-en-1- one |
| 134 | 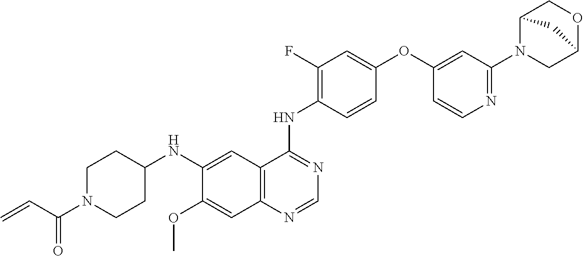 [SMILES: C=CC(=O)N1CCC(Nc2cc3c(Nc4ccc(Oc5ccnc(N6C[C@H]7C[C@@H]6CO7)c5)cc4F)ncnc3cc2OC)CC1] | 1-(4-((4-((4-((2- ((1R,4R)-2-oxa-5- azabicyclo[2.2.1] heptan-5-yl)pyridin- 4-yl)oxy)-2-fluorophenyl) amino)-7- methoxyquinazolin-6- yl)amino)piperidin- 1-yl)prop-2-en-1- one |
| 135 | 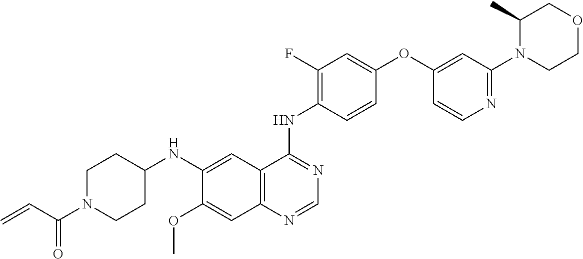 [SMILES: C=CC(=O)N1CCC(Nc2cc3c(Nc4ccc(Oc5ccnc(N6CCOC[C@@H]6C)c5)cc4F)ncnc3cc2OC)CC1] | (S)-1-(4-((4-((2- fluoro-4-((2-(3- methylmorpholino)pyridin-4- yl)oxy)phenyl)amino)-7- methoxyquinazolin-6- yl)amino)piperidin- 1-yl)prop-2-en-1- one |
| 136 | 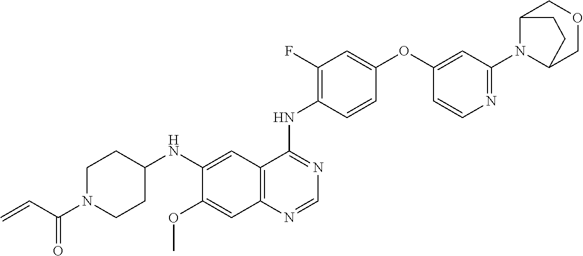 [SMILES: C=CC(=O)N1CCC(Nc2cc3c(Nc4ccc(Oc5ccnc(N6C7CCC6COC7)c5)cc4F)ncnc3cc2OC)CC1] | 1-(4-((4-((4-((2-(3-oxa-8- azabicyclo[3.2.1] octan-8-yl)pyridin- 4-yl)oxy)-2-fluorophenyl) amino)-7- methoxyquinazolin-6- yl)amino)piperidin- 1-yl)prop-2-en-1- one |
| 137 | 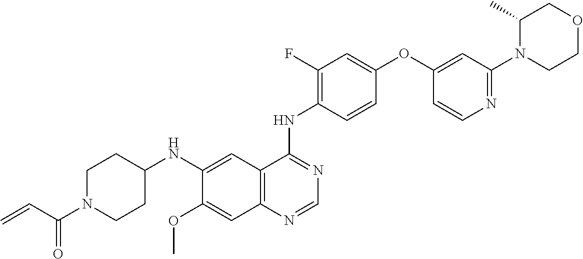 [SMILES: C=CC(=O)N1CCC(Nc2cc3c(Nc4ccc(Oc5ccnc(N6CCOC[C@H]6C)c5)cc4F)ncnc3cc2OC)CC1] | (R)-1-(4-((4-((2- fluoro-4-((2-(3- methylmorpholino)pyridin-4- yl)oxy)phenyl)amino)-7- methoxyquinazolin-6- yl)amino)piperidin- 1-yl)prop-2-en-1- one |
| 138 | 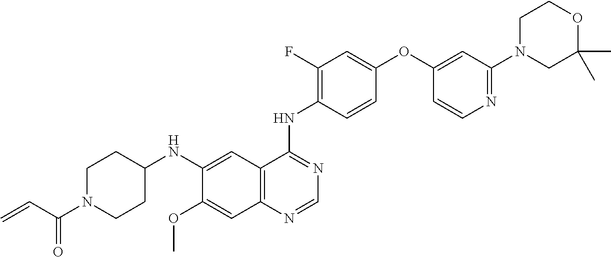 [SMILES: C=CC(=O)N1CCC(Nc2cc3c(Nc4ccc(Oc5ccnc(N6CCOC(C)(C)C6)c5)cc4F)ncnc3cc2OC)CC1] | 1-(4-((4-((4-((2-(2,2- dimethylmorpholino) pyridin-4- yl)oxy)-2-fluorophenyl) amino)-7- methoxyquinazolin-6- yl)amino)piperidin- 1-yl)prop-2-en-1- one |
| 139 | 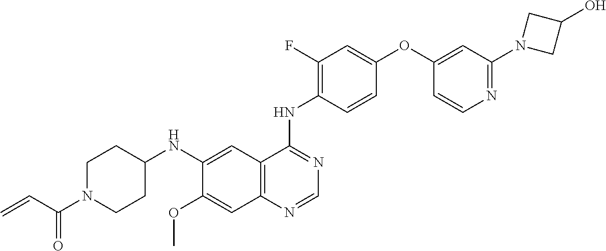 [SMILES: C=CC(=O)N1CCC(Nc2cc3c(Nc4ccc(Oc5ccnc(N6CC(O)C6)c5)cc4F)ncnc3cc2OC)CC1] | 1-(4-((4-((2-fluoro-4-((2-(3- hydroxyazetidin-1- yl)pyridin-4- yl)oxy)phenyl)amino)-7- methoxyquinazolin-6- yl)amino)piperidin- 1-yl)prop-2-en-1- one |
| 140 | 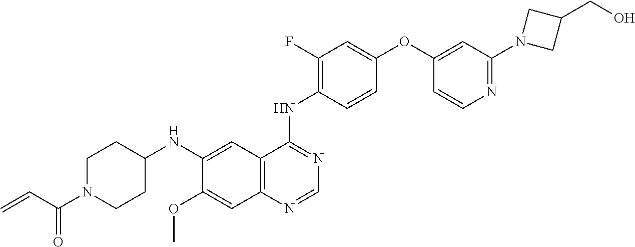 [SMILES: C=CC(=O)N1CCC(Nc2cc3c(Nc4ccc(Oc5ccnc(N6CC(CO)C6)c5)cc4F)ncnc3cc2OC)CC1] | 1-(4-((4-((2-fluoro-4-((2-(3- (hydroxymethyl) azetidin-1-yl)pyridin- 4-yl)oxy)phenyl)amino)-7- methoxyquinazolin-6- yl)amino)piperidin- 1-yl)prop-2-en-1- one |
| 141 | 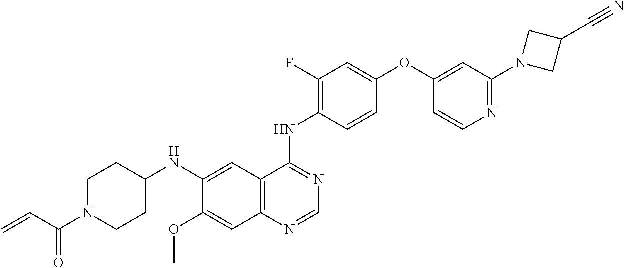 [SMILES: C=CC(=O)N1CCC(Nc2cc3c(Nc4ccc(Oc5ccnc(N6CC(C#N)C6)c5)cc4F)ncnc3cc2OC)CC1] | 1-(4-(4-((6-((1- acryloylpiperidin-4- yl)amino)-7- methoxyquinazolin-4- yl)amino)-3- fluorophenoxy)pyridin-2- yl)azetidine-3-carbonitrile |
| 142 | 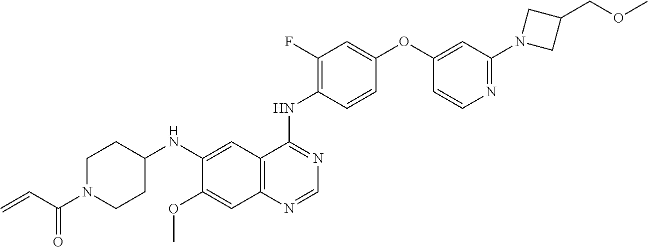 [SMILES: C=CC(=O)N1CCC(Nc2cc3c(Nc4ccc(Oc5ccnc(N6CC(COC)C6)c5)cc4F)ncnc3cc2OC)CC1] | 1-(4-((4-((2-fluoro-4-((2-(3- (methoxymethyl)azetidin-1- yl)pyridin-4-yl)oxy) phenyl)amino)-7- methoxyquinazolin-6- yl)amino)piperidin- 1-yl)prop-2-en-1- one |
| 143 | 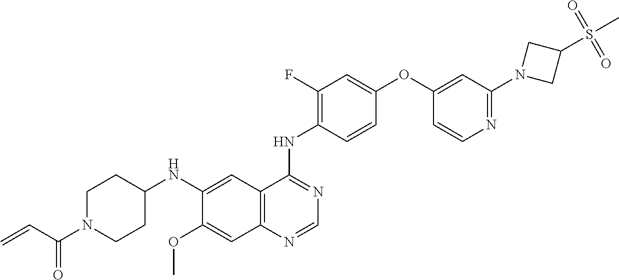 [SMILES: C=CC(=O)N1CCC(Nc2cc3c(Nc4ccc(Oc5ccnc(N6CC(S(C)(=O)=O)C6)c5)cc4F)ncnc3cc2OC)CC1] | 1-(4-((4-((2-fluoro-4-((2-(3- (methylsulfonyl) azetidin-1-yl)pyridin- 4-yl)oxy)phenyl)amino)-7- methoxyquinazolin-6- yl)amino)piperidin- 1-yl)prop-2-en-1- one |
| 144 | 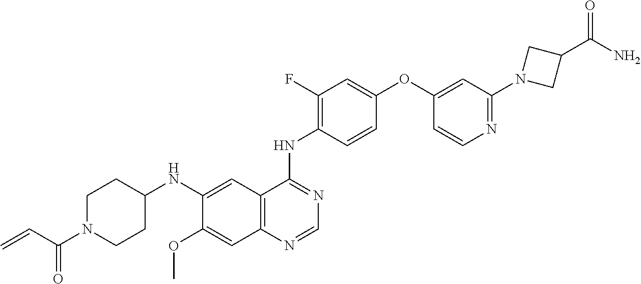 [SMILES: C=CC(=O)N1CCC(Nc2cc3c(Nc4ccc(Oc5ccnc(N6CC(C(N)=O)C6)c5)cc4F)ncnc3cc2OC)CC1] | 1-(4-(4-((6-((1- acryloylpiperidin-4- yl)amino)-7- methoxyquinazolin-4- yl)amino)-3- fluorophenoxy)pyridin-2- yl)azetidine-3-carboxamide |
| 145 | 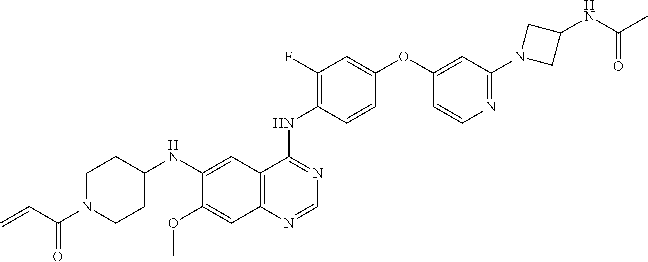 [SMILES: C=CC(=O)N1CCC(Nc2cc3c(Nc4ccc(Oc5ccnc(N6CC(NC(C)=O)C6)c5)cc4F)ncnc3cc2OC)CC1] | N-(1-(4-(4-((6-((1- acryloylpiperidin- 4-yl)amino)-7- methoxyquinazolin-4- yl)amino)-3- fluorophenoxy)pyridin-2- yl)azetidin-3-yl)acetamide |
| 146 | 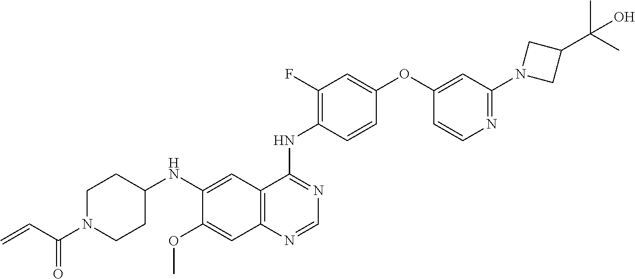 [SMILES: C=CC(=O)N1CCC(Nc2cc3c(Nc4ccc(Oc5ccnc(N6CC(C(C)(C)O)C6)c5)cc4F)ncnc3cc2OC)CC1] | 1-(4-((4-((2-fluoro- 4-((2-(3-(2- hydroxypropan- 2-yl)azetidin-1- yl)pyridin-4-yl)oxy) phenyl)amino)-7- methoxyquinazolin-6- yl)amino)piperidin- 1-yl)prop-2-en-1- one |
| 147 | 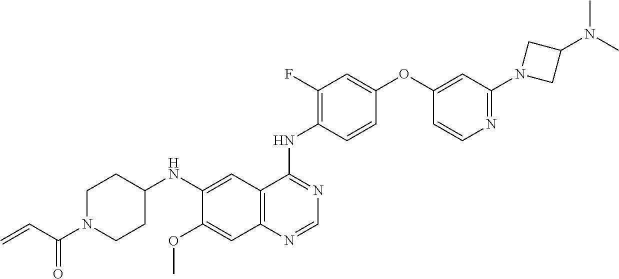 [SMILES: C=CC(=O)N1CCC(Nc2cc3c(Nc4ccc(Oc5ccnc(N6CC(N(C)C)C6)c5)cc4F)ncnc3cc2OC)CC1] | 1-(4-((4-((4-((2-(3- (dimethylamino) azetidin-1-yl)pyridin- 4-yl)oxy)-2-fluorophenyl) amino)-7- methoxyquinazolin-6- yl)amino)piperidin- 1-yl)prop-2-en-1- one |
| 148 | 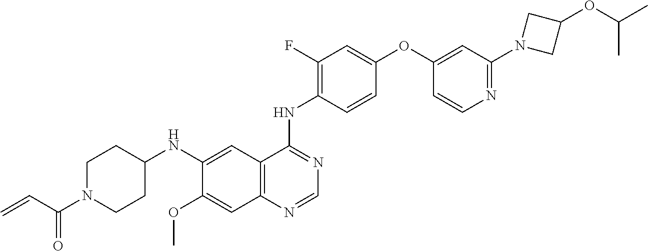 [SMILES: C=CC(=O)N1CCC(Nc2cc3c(Nc4ccc(Oc5ccnc(N6CC(OC(C)C)C6)c5)cc4F)ncnc3cc2OC)CC1] | 1-(4-((4-((2-fluoro-4-((2-(3- isopropoxyazetidin- 1-yl)pyridin-4- yl)oxy)phenyl)amino)-7- methoxyquinazolin-6- yl)amino)piperidin- 1-yl)prop-2-en-1- one |
| 149 | 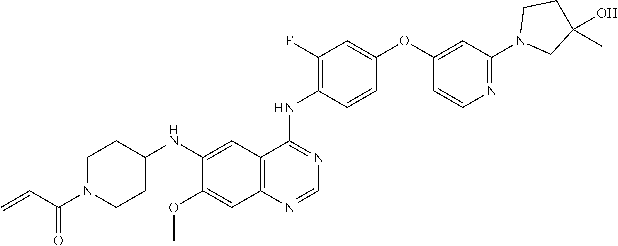 [SMILES: C=CC(=O)N1CCC(Nc2cc3c(Nc4ccc(Oc5ccnc(N6CCC(C)(O)C6)c5)cc4F)ncnc3cc2OC)CC1] | 1-(4-((4-((2-fluoro- 4-((2-(3-hydroxy- 3-methylpyrrolidin- 1-yl)pyridin-4- yl)oxy)phenyl)amino)-7- methoxyquinazolin-6- yl)amino)piperidin- 1-yl)prop-2-en-1- one |
| 150 | 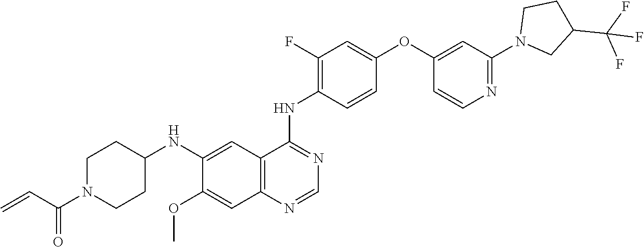 [SMILES: C=CC(=O)N1CCC(Nc2cc3c(Nc4ccc(Oc5ccnc(N6CCC(C(F)(F)F)C6)c5)cc4F)ncnc3cc2OC)CC1] | 1-(4-((4-((2-fluoro-4-((2-(3- (trifluoromethyl) pyrrolidin-1- yl)pyridin-4-yl)oxy) phenyl)amino)-7- methoxyquinazolin-6- yl)amino)piperidin- 1-yl)prop-2-en-1- one |
| 151 | 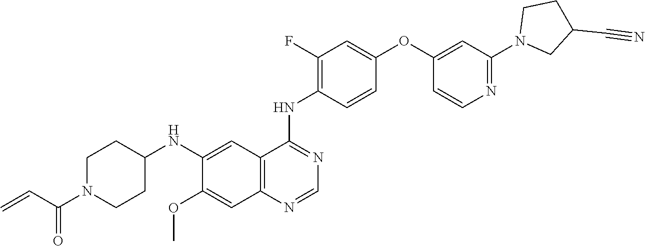 [SMILES: C=CC(=O)N1CCC(Nc2cc3c(Nc4ccc(Oc5ccnc(N6CCC(C#N)C6)c5)cc4F)ncnc3cc2OC)CC1] | 1-(4-(4-((6-((1- acryloylpiperidin-4- yl)amino)-7- methoxyquinazolin-4- yl)amino)-3- fluorophenoxy)pyridin-2- yl)pyrrolidine-3-carbonitrile |
| 152 | 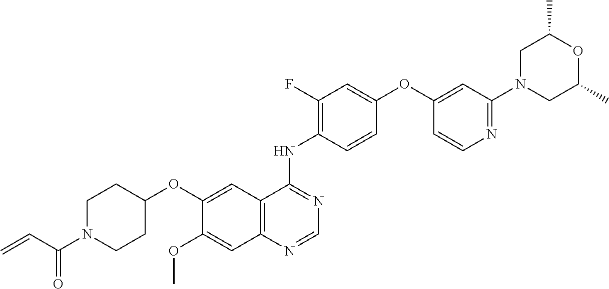 [SMILES: C=CC(=O)N1CCC(Oc2cc3c(Nc4ccc(Oc5ccnc(N6C[C@@H](C)O[C@@H](C)C6)c5)cc4F)ncnc3cc2OC)CC1] | 1-(4-((4-((4-((2-((2R,6S)-2,6- dimethylmorpholino) pyridin-4- yl)oxy)-2- fluorophenyl)amino)-7- methoxyquinazolin-6- yl)oxy)piperidin- 1-yl)prop-2-en-1- one |
| 153 | 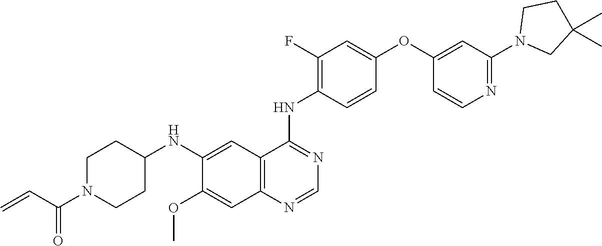 [SMILES: C=CC(=O)N1CCC(Nc2cc3c(Nc4ccc(Oc5ccnc(N6CCC(C)(C)C6)c5)cc4F)ncnc3cc2OC)CC1] | 1-(4-((4-((4-((2-(3,3- dimethylpyrrolidin- 1-yl)pyridin-4- yl)oxy)-2- fluorophenyl)amino)-7- methoxyquinazolin-6- yl)amino)piperidin- 1-yl)prop-2-en-1- one |
| 154 | 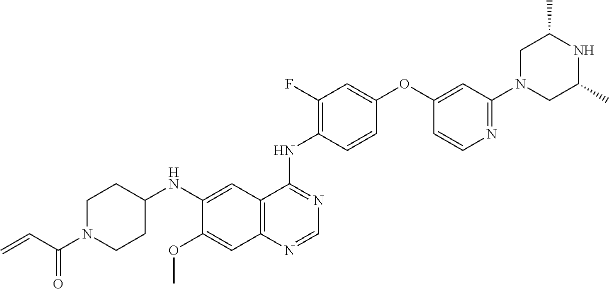 [SMILES: C=CC(=O)N1CCC(Nc2cc3c(Nc4ccc(Oc5ccnc(N6C[C@@H](C)N[C@@H](C)C6)c5)cc4F)ncnc3cc2OC)CC1] | 1-(4-((4-((4-((2-((3R,5S)-3,5- dimethylpiperazin- 1-yl)pyridin-4- yl)oxy)-2-fluorophenyl) amino)-7- methoxyquinazolin-6- yl)amino)piperidin- 1-yl)prop-2-en-1- one |
| 155 | 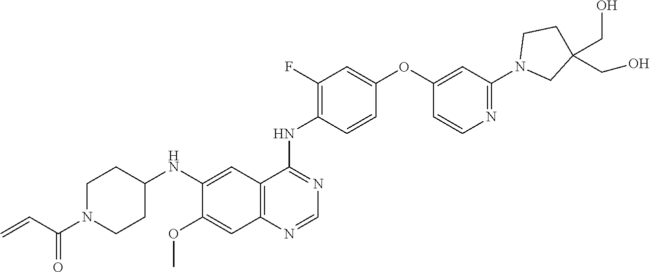 [SMILES: C=CC(=O)N1CCC(Nc2cc3c(Nc4ccc(Oc5ccnc(N6CCC(CO)(CO)C6)c5)cc4F)ncnc3cc2OC)CC1] | 1-(4-((4-((4-((2-(3,3- bis(hydroxymethyl) pyrrolidin-1- yl)pyridin-4-yl)oxy)-2- fluorophenyl)amino)-7- methoxyquinazolin-6- yl)amino)piperidin- 1-yl)prop-2-en-1- one |
| 156 | 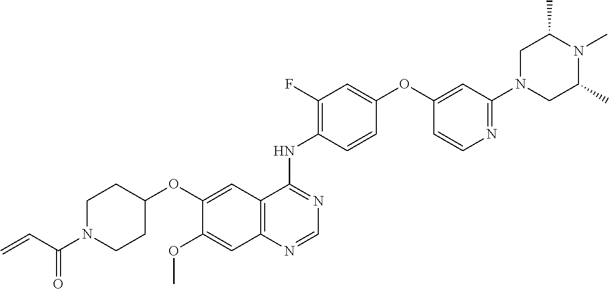 [SMILES: C=CC(=O)N1CCC(Oc2cc3c(Nc4ccc(Oc5ccnc(N6C[C@@H](C)N(C)[C@@H](C)C6)c5)cc4F)ncnc3cc2OC)CC1] | 1-(4-((4-((2-fluoro- 4-((2-((3R,5S)- 3,4,5-trimethylpiperazin- 1-yl)pyridin- 4-yl)oxy)phenyl)amino)-7- methoxyquinazolin-6- yl)oxy)piperidin- 1-yl)prop-2-en-1- one |
| 157 | 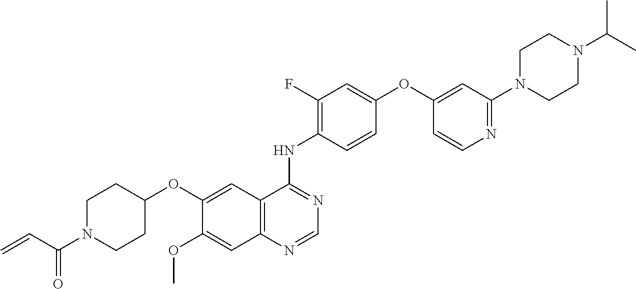 [SMILES: C=CC(=O)N1CCC(Oc2cc3c(Nc4ccc(Oc5ccnc(N6CCN(C(C)C)CC6)c5)cc4F)ncnc3cc2OC)CC1] | 1-(4-((4-((2-fluoro-4-((2-(4- isopropylpiperazin- 1-yl)pyridin-4- yl)oxy)phenyl)amino)-7- methoxyquinazolin-6- yl)oxy)piperidin- 1-yl)prop-2-en-1- one |
| 158 | 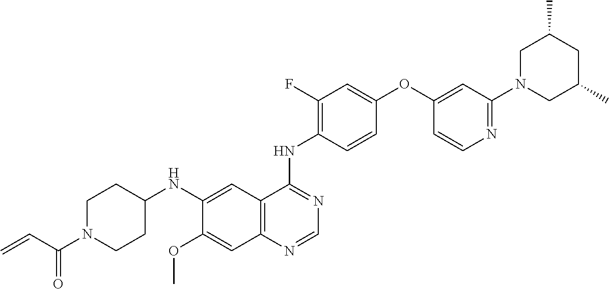 [SMILES: C=CC(=O)N1CCC(Nc2cc3c(Nc4ccc(Oc5ccnc(N6C[C@H](C)C[C@H](C)C6)c5)cc4F)ncnc3cc2OC)CC1] | 1-(4-((4-((4-((2-((3S,5R)-3,5- dimethylpiperidin- 1-yl)pyridin-4- yl)oxy)-2- fluorophenyl)amino)-7- methoxyquinazolin-6- yl)amino)piperidin- 1-yl)prop-2-en-1- one |
| 159 | 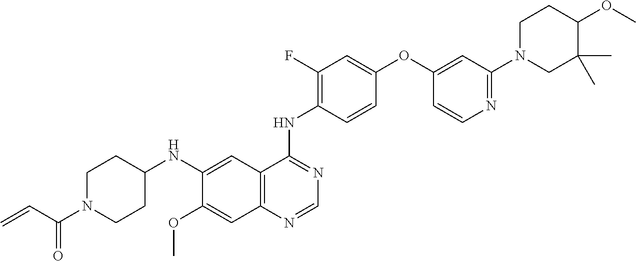 [SMILES: C=CC(=O)N1CCC(Nc2cc3c(Nc4ccc(Oc5ccnc(N6CCC(OC)C(C)(C)C6)c5)cc4F)ncnc3cc2OC)CC1] | 1-(4-((4-((2-fluoro-4- ((2-(4-methoxy- 3,3-dimethylpiperidin- 1-yl)pyridin-4- yl)oxy)phenyl)amino)-7- methoxyquinazolin-6- yl)amino)piperidin- 1-yl)prop-2-en-1- one |
| 160 | 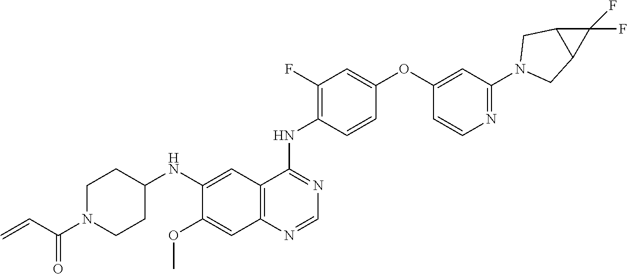 [SMILES: C=CC(=O)N1CCC(Nc2cc3c(Nc4ccc(Oc5ccnc(N6CC7C(C6)C7(F)F)c5)cc4F)ncnc3cc2OC)CC1] | 1-(4-((4-((4-((2- (6,6-difluoro-3- azabicyclo[3.1.0] hexan-3-yl)pyridin- 4-yl)oxy)-2- fluorophenyl)amino)-7- methoxyquinazolin-6- yl)amino)piperidin- 1-yl)prop-2-en-1- one |
| 161 | 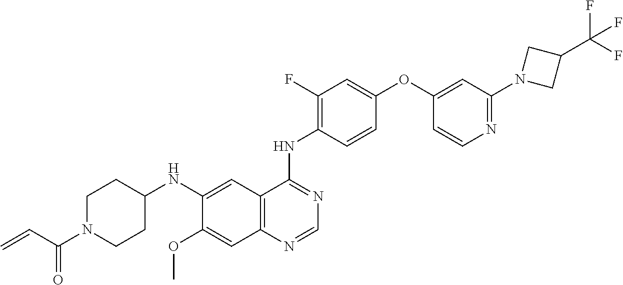 [SMILES: C=CC(=O)N1CCC(Nc2cc3c(Nc4ccc(Oc5ccnc(N6CC(C(F)(F)F)C6)c5)cc4F)ncnc3cc2OC)CC1] | 1-(4-((4-((2-fluoro-4-((2-(3- (trifluoromethyl) azetidin-1-yl)pyridin- 4-yl)oxy)phenyl)amino)-7- methoxyquinazolin-6- yl)amino)piperidin- 1-yl)prop-2-en-1- one |
| 162 | 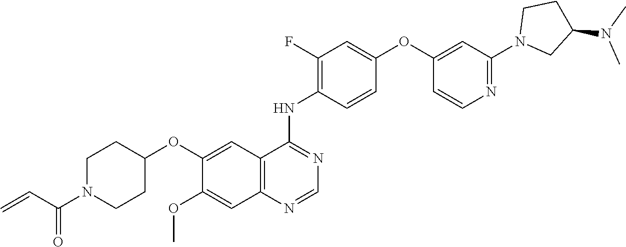 [SMILES: C=CC(=O)N1CCC(Oc2cc3c(Nc4ccc(Oc5ccnc(N6CC[C@@H](N(C)C)C6)c5)cc4F)ncnc3cc2OC)CC1] | (R)-1-(4-((4-((4-((2-(3- (dimethylamino) pyrrolidin-1- yl)pyridin-4-yl)oxy)-2- fluorophenyl)amino)-7- methoxyquinazolin-6- yl)oxy)piperidin- 1-yl)prop-2-en-1- one |
| 163 | 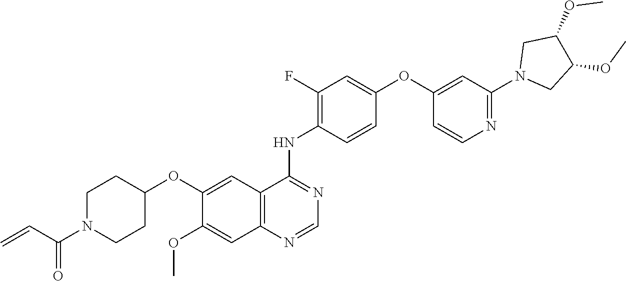 [SMILES: C=CC(=O)N1CCC(Oc2cc3c(Nc4ccc(Oc5ccnc(N6C[C@H](OC)[C@H](OC)C6)c5)cc4F)ncnc3cc2OC)CC1] | 1-(4-((4-((4-((2-((3R,4S)-3,4- dimethoxypyrrolidin- 1-yl)pyridin-4- yl)oxy)-2- fluorophenyl)amino)-7- methoxyquinazolin-6- yl)oxy)piperidin- 1-yl)prop-2-en-1- one |
| 164 | 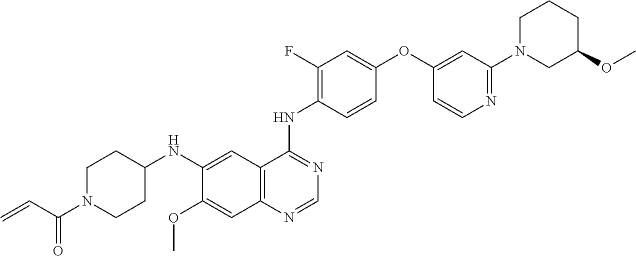 [SMILES: C=CC(=O)N1CCC(Nc2cc3c(Nc4ccc(Oc5ccnc(N6CCC[C@@H](OC)C6)c5)cc4F)ncnc3cc2OC)CC1] | (R)-1-(4-((4-((2- fluoro-4-((2-(3- methoxypiperidin- 1-yl)pyridin-4- yl)oxy)phenyl)amino)-7- methoxyquinazolin-6- yl)amino)piperidin- 1-yl)prop-2-en-1- one |
| 165 | 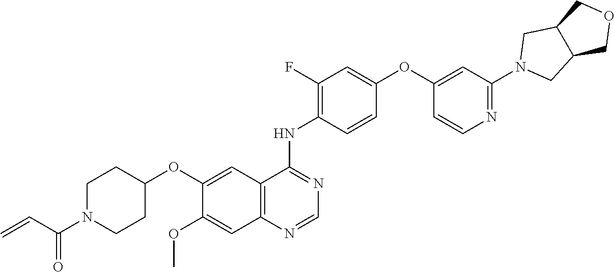 [SMILES: C=CC(=O)N1CCC(Oc2cc3c(Nc4ccc(Oc5ccnc(N6C[C@H]7COC[C@H]7C6)c5)cc4F)ncnc3cc2OC)CC1] | 1-(4-((4-((2-fluoro- 4-((2-((3aR,6aS)- tetrahydro-1H- furo[3,4-c]pyrrol- 5(3H)-yl)pyridin-4- yl)oxy)phenyl)amino)-7- methoxyquinazolin-6- yl)oxy)piperidin- 1-yl)prop-2-en-1- one |
| 166 | 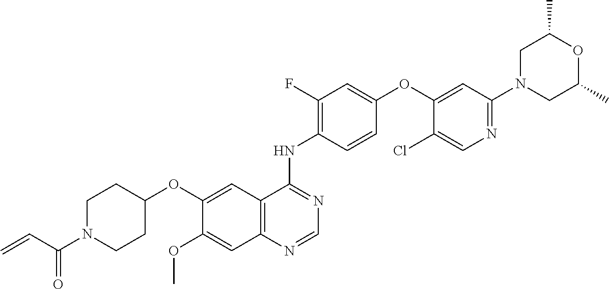 [SMILES: C=CC(=O)N1CCC(Oc2cc3c(Nc4ccc(Oc5cc(N6C[C@@H](C)O[C@@H](C)C6)ncc5Cl)cc4F)ncnc3cc2OC)CC1] | 1-(4-((4-((4-((5- chloro-2-((2R,6S)- 2,6-dimethylmorpholino) pyridin-4- yl)oxy)-2- fluorophenyl)amino)-7- methoxyquinazolin-6- yl)oxy)piperidin- 1-yl)prop-2-en-1- one |
| 167 | 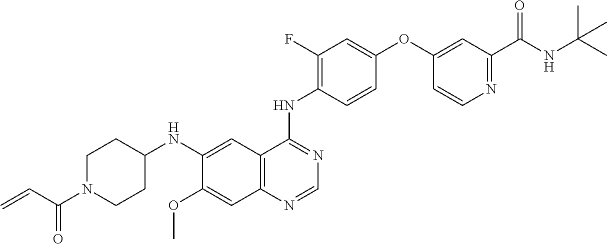 [SMILES: C=CC(=O)N1CCC(Nc2cc3c(Nc4ccc(Oc5ccnc(C(=O)NC(C)(C)C)c5)cc4F)ncnc3cc2OC)CC1] | 4-(4-((6-((1- acryloylpiperidin-4- yl)amino)-7- methoxyquinazolin-4- yl)amino)-3- fluorophenoxy)-N-(tert- butyl)picolinamide |
| 168 | 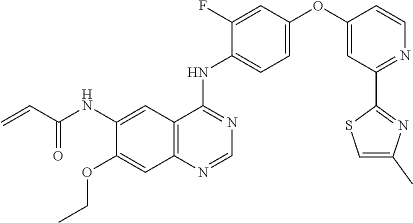 [SMILES: C=CC(=O)Nc1cc2c(Nc3ccc(Oc4ccnc(-c5nc(C)cs5)c4)cc3F)ncnc2cc1OCC] | N-(7-ethoxy-4- ((2-fluoro-4-((2-(4- methylthiazol-2-yl)pyridin-4- yl)oxy)phenyl)amino) quinazolin-6- yl)acrylamide |
| 169 | 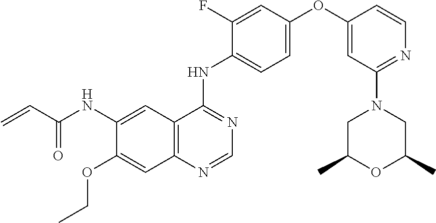 [SMILES: C=CC(=O)Nc1cc2c(Nc3ccc(Oc4ccnc(N5C[C@@H](C)O[C@@H](C)C5)c4)cc3F)ncnc2cc1OCC] | N-(4-((4-((2-((2S,6R)-2,6- dimethylmorpholino) pyridin-4- yl)oxy)- 2-fluorophenyl)amino)-7- ethoxyquinazolin- 6-yl)acrylamide |
| 170 | 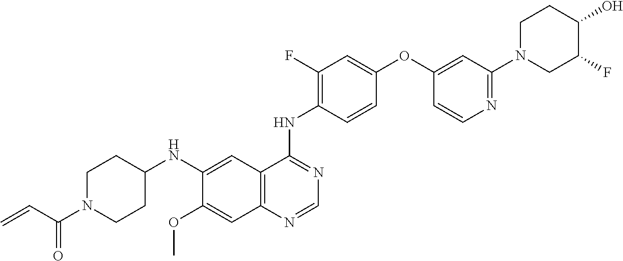 [SMILES: C=CC(=O)N1CCC(Nc2cc3c(Nc4ccc(Oc5ccnc(N6CC[C@H](O)[C@H](F)C6)c5)cc4F)ncnc3cc2OC)CC1] | 1-(4-((4-((2-fluoro- 4-((2-((3R,4S)-3- fluoro-4-hydroxypiperidin-1- yl)pyridin-4-yl)oxy) phenyl)amino)-7- methoxyquinazolin-6- yl)amino)piperidin- 1-yl)prop-2-en-1- one |
| 171 | 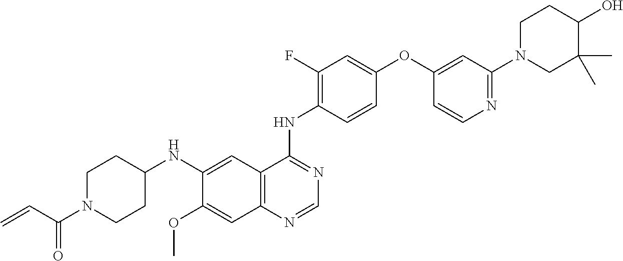 [SMILES: C=CC(=O)N1CCC(Nc2cc3c(Nc4ccc(Oc5ccnc(N6CCC(O)C(C)(C)C6)c5)cc4F)ncnc3cc2OC)CC1] | 1-(4-((4-((2-fluoro- 4-((2-(4-hydroxy- 3,3-dimethylpiperidin- 1-yl)pyridin-4- yl)oxy)phenyl)amino)-7- methoxyquinazolin-6- yl)amino)piperidin- 1-yl)prop-2-en-1- one |
| 172 | 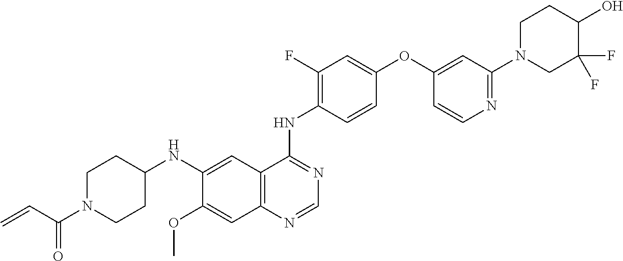 [SMILES: C=CC(=O)N1CCC(Nc2cc3c(Nc4ccc(Oc5ccnc(N6CCC(O)C(F)(F)C6)c5)cc4F)ncnc3cc2OC)CC1] | 1-(4-((4-((4-((2- (3,3-difluoro-4- hydroxypiperidin- 1-yl)pyridin-4- yl)oxy)-2- fluorophenyl)amino)-7- methoxyquinazolin-6- yl)amino)piperidin- 1-yl)prop-2-en-1- one |
| 173 | 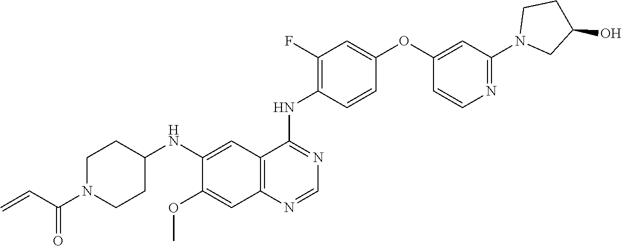 [SMILES: C=CC(=O)N1CCC(Nc2cc3c(Nc4ccc(Oc5ccnc(N6CC[C@@H](O)C6)c5)cc4F)ncnc3cc2OC)CC1] | (R)-1-(4-((4-((2- fluoro-4-((2-(3- hydroxypyrrolidin- 1-yl)pyridin-4- yl)oxy)phenyl)amino)-7- methoxyquinazolin-6- yl)amino)piperidin- 1-yl)prop-2-en-1- one |
| 174 | 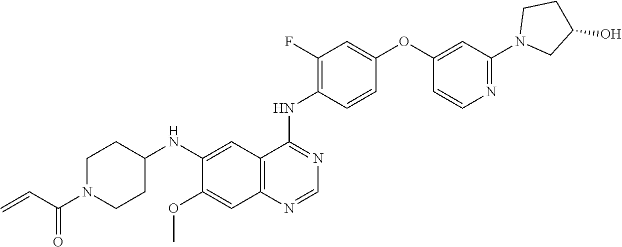 [SMILES: C=CC(=O)N1CCC(Nc2cc3c(Nc4ccc(Oc5ccnc(N6CC[C@H](O)C6)c5)cc4F)ncnc3cc2OC)CC1] | (S)-1-(4-((4-((2- fluoro-4-((2-(3- hydroxypyrrolidin- 1-yl)pyridin-4- yl)oxy)phenyl)amino)-7- methoxyquinazolin-6- yl)amino)piperidin- 1-yl)prop-2-en-1- one |
| 175 | 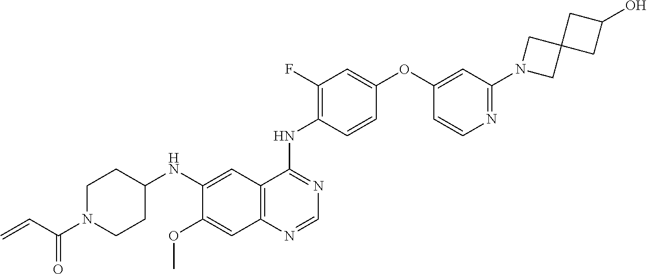 [SMILES: C=CC(=O)N1CCC(Nc2cc3c(Nc4ccc(Oc5ccnc(N6CC7(CC(O)C7)C6)c5)cc4F)ncnc3cc2OC)CC1] | 1-(4-((4-((2-fluoro- 4-((2-(6-hydroxy- 2-azaspiro[3.3]heptan- 2-yl)pyridin-4- yl)oxy)phenyl)amino)-7- methoxyquinazolin-6- yl)amino)piperidin- 1-yl)prop-2-en-1- one |
| 176 | 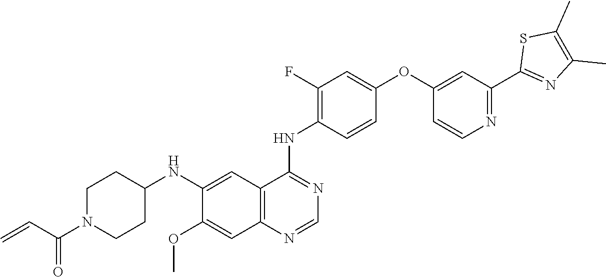 [SMILES: C=CC(=O)N1CCC(Nc2cc3c(Nc4ccc(Oc5ccnc(-c6nc(C)c(C)s6)c5)cc4F)ncnc3cc2OC)CC1] | 1-(4-((4-((4-((2- (4,5-dimethylthiazol- 2-yl)pyridin-4-yl)oxy)-2- fluorophenyl)amino)-7- methoxyquinazolin-6- yl)amino)piperidin- 1-yl)prop-2-en-1- one |
| 177 | 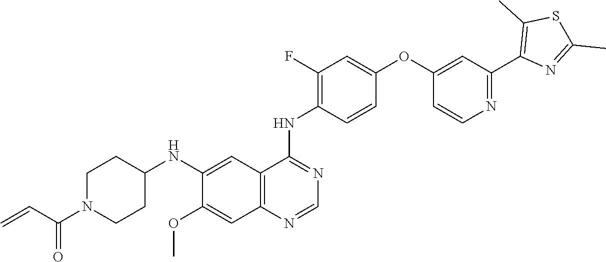 [SMILES: C=CC(=O)N1CCC(Nc2cc3c(Nc4ccc(Oc5ccnc(-c6nc(C)sc6C)c5)cc4F)ncnc3cc2OC)CC1] | 1-(4-((4-((4-((2- (2,5-dimethylthiazol- 4-yl)pyridin-4-yl)oxy)-2- fluorophenyl)amino)-7- methoxyquinazolin-6- yl)amino)piperidin- 1-yl)prop-2-en-1- one |
| 178 | 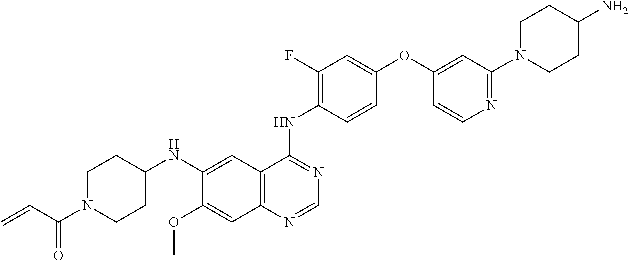 [SMILES: C=CC(=O)N1CCC(Nc2cc3c(Nc4ccc(Oc5ccnc(N6CCC(N)CC6)c5)cc4F)ncnc3cc2OC)CC1] | 1-(4-((4-((4-((2- (4-aminopiperidin-1- yl)pyridin-4-yl)oxy)-2- fluorophenyl)amino)-7- methoxyquinazolin-6- yl)amino)piperidin- 1-yl)prop-2-en-1- one |
| 179 | 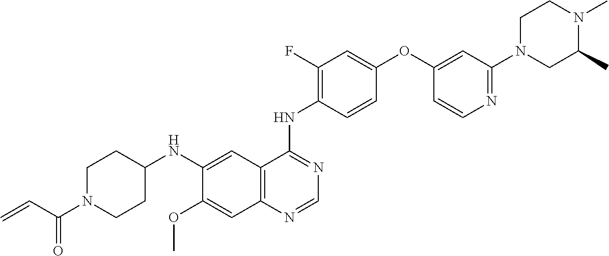 [SMILES: C=CC(=O)N1CCC(Nc2cc3c(Nc4ccc(Oc5ccnc(N6CCN(C)[C@@H](C)C6)c5)cc4F)ncnc3cc2OC)CC1] | (S)-1-(4-((4-((4-((2-(3,4- dimethylpiperazin- 1-yl)pyridin-4- yl)oxy)-2- fluorophenyl)amino)-7- methoxyquinazolin-6- yl)amino)piperidin- 1-yl)prop-2-en-1- one |
| 180 | 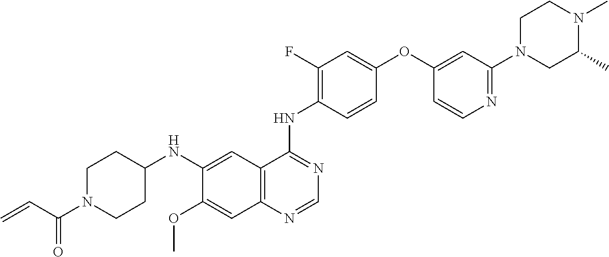 [SMILES: C=CC(=O)N1CCC(Nc2cc3c(Nc4ccc(Oc5ccnc(N6CCN(C)[C@H](C)C6)c5)cc4F)ncnc3cc2OC)CC1] | (R)-1-(4-((4-((4-((2-(3,4- dimethylpiperazin- 1-yl)pyridin-4- yl)oxy)-2-fluorophenyl) amino)-7- methoxyquinazolin-6- yl)amino)piperidin- 1-yl)prop-2-en-1- one |
| 181 | 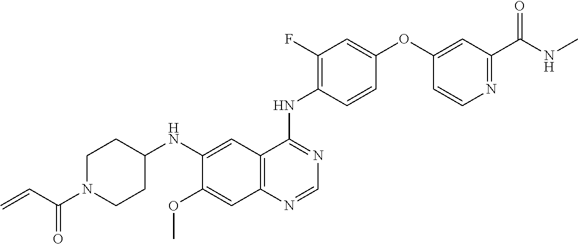 [SMILES: C=CC(=O)N1CCC(Nc2cc3c(Nc4ccc(Oc5ccnc(C(=O)NC)c5)cc4F)ncnc3cc2OC)CC1] | 4-(4-((6-((1- acryloylpiperidin-4- yl)amino)-7- methoxyquinazolin-4- yl)amino)-3- fluorophenoxy)-N- methylpicolinamide |
| 182 | 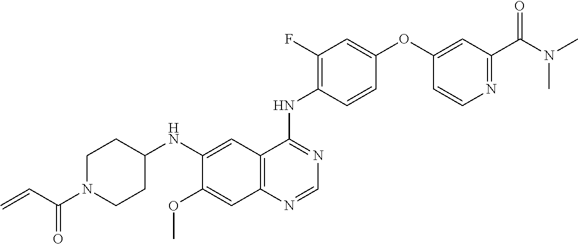 [SMILES: C=CC(=O)N1CCC(Nc2cc3c(Nc4ccc(Oc5ccnc(C(=O)N(C)C)c5)cc4F)ncnc3cc2OC)CC1] | 4-(4-((6-((1- acryloylpiperidin-4- yl)amino)-7- methoxyquinazolin-4- yl)amino)-3- fluorophenoxy)-N,N- dimethylpicolinamide |
| 183 | 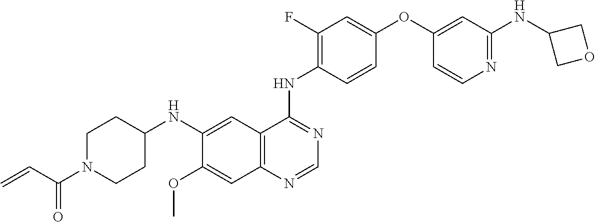 [SMILES: C=CC(=O)N1CCC(Nc2cc3c(Nc4ccc(Oc5ccnc(NC6COC6)c5)cc4F)ncnc3cc2OC)CC1] | 1-(4-((4-((2-fluoro- 4-((2-(oxetan-3- ylamino)pyridin-4- yl)oxy)phenyl)amino)-7- methoxyquinazolin-6- yl)amino)piperidin- 1-yl)prop-2-en-1- one |
| 184 | 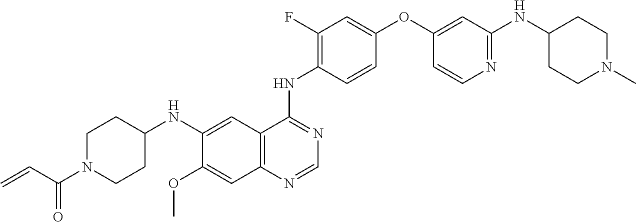 [SMILES: C=CC(=O)N1CCC(Nc2cc3c(Nc4ccc(Oc5ccnc(NC6CCN(C)CC6)c5)cc4F)ncnc3cc2OC)CC1] | 1-(4-((4-((2-fluoro-4-((2-((1- methylpiperidin-4- yl)amino)pyridin- 4-yl)oxy)phenyl)amino)-7- methoxyquinazolin-6- yl)amino)piperidin- 1-yl)prop-2-en-1- one |
| 185 | 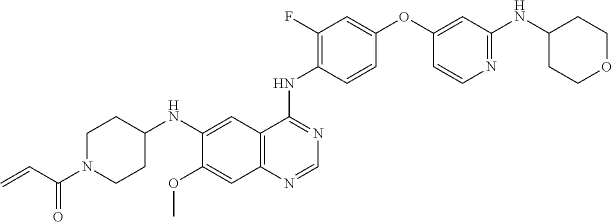 [SMILES: C=CC(=O)N1CCC(Nc2cc3c(Nc4ccc(Oc5ccnc(NC6CCOCC6)c5)cc4F)ncnc3cc2OC)CC1] | 1-(4-((4-((2-fluoro- 4-((2-((tetrahydro- 2H-pyran-4-yl) amino)pyridin-4- yl)oxy)phenyl)amino)-7- methoxyquinazolin-6- yl)amino)piperidin- 1-yl)prop-2-en-1- one |
| 186 | 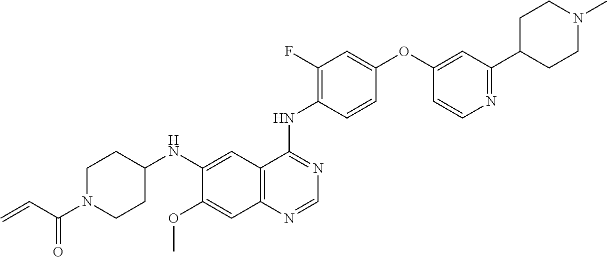 [SMILES: C=CC(=O)N1CCC(Nc2cc3c(Nc4ccc(Oc5ccnc(C6CCN(C)CC6)c5)cc4F)ncnc3cc2OC)CC1] | 1-(4-((4-((2-fluoro-4-((2-(1- methylpiperidin- 4-yl)pyridin-4- yl)oxy)phenyl)amino)-7- methoxyquinazolin-6- yl)amino)piperidin- 1-yl)prop-2-en-1- one |
| 187 | 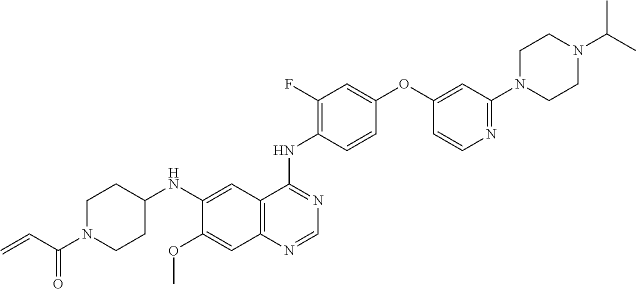 [SMILES: C=CC(=O)N1CCC(Nc2cc3c(Nc4ccc(Oc5ccnc(N6CCN(C(C)C)CC6)c5)cc4F)ncnc3cc2OC)CC1] | 1-(4-((4-((2-fluoro-4-((2-(4- isopropylpiperazin- 1-yl)pyridin-4- yl)oxy)phenyl)amino)-7- methoxyquinazolin-6- yl)amino)piperidin- 1-yl)prop-2-en-1- one |
| 188 | 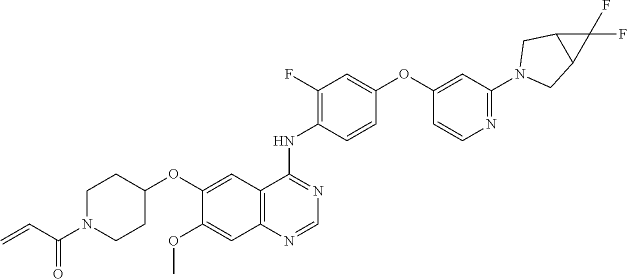 [SMILES: C=CC(=O)N1CCC(Oc2cc3c(Nc4ccc(Oc5ccnc(N6CC7C(C6)C7(F)F)c5)cc4F)ncnc3cc2OC)CC1] | 1-(4-((4-((4-((2- (6,6-difluoro-3- azabicyclo[3.1.0] hexan-3-yl)pyridin- 4-yl)oxy)-2- fluorophenyl)amino)-7- methoxyquinazolin-6- yl)oxy)piperidin- 1-yl)prop-2-en-1- one |
| 189 | 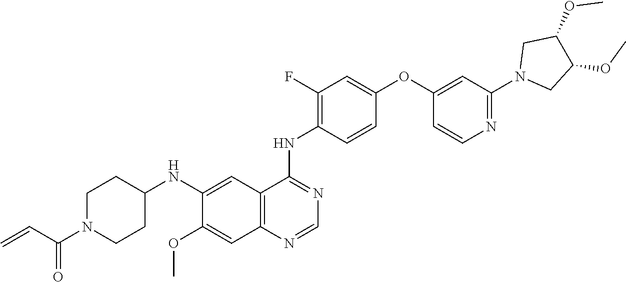 [SMILES: C=CC(=O)N1CCC(Nc2cc3c(Nc4ccc(Oc5ccnc(N6C[C@H](OC)[C@H](OC)C6)c5)cc4F)ncnc3cc2OC)CC1] | 1-(4-((4-((4-((2-((3R,4S)-3,4- dimethoxypyrrolidin- 1-yl)pyridin-4- yl)oxy)-2- fluorophenyl)amino)-7- methoxyquinazolin-6- yl)amino)piperidin- 1-yl)prop-2-en-1- one |
| 190 | 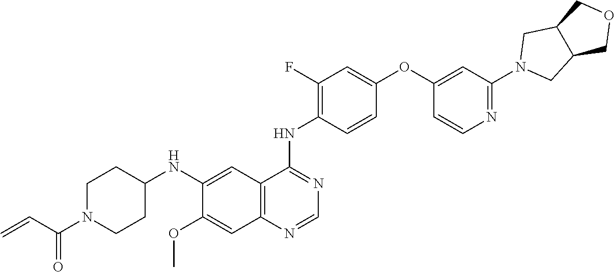 [SMILES: C=CC(=O)N1CCC(Nc2cc3c(Nc4ccc(Oc5ccnc(N6C[C@H]7COC[C@H]7C6)c5)cc4F)ncnc3cc2OC)CC1] | 1-(4-((4-((2-fluoro- 4-((2-((3aR,6aS)- tetrahydro-1H- furo[3,4-c]pyrrol- 5(3H)-yl)pyridin-4- yl)oxy)phenyl)amino)-7- methoxyquinazolin-6- yl)amino)piperidin- 1-yl)prop-2-en-1- one |
| 191 | 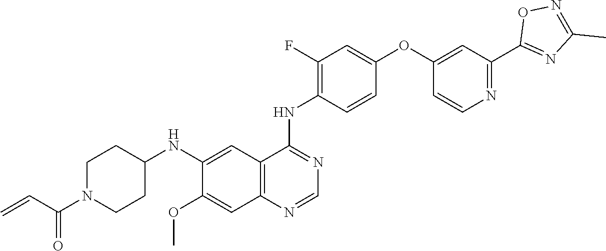 [SMILES: C=CC(=O)N1CCC(Nc2cc3c(Nc4ccc(Oc5ccnc(-c6nc(C)no6)c5)cc4F)ncnc3cc2OC)CC1] | 1-(4-((4-((2-fluoro- 4-((2-(3-methyl- 1,2,4-oxadiazol- 5-yl)pyridin-4- yl)oxy)phenyl)amino)-7- methoxyquinazolin-6- yl)amino)piperidin- 1-yl)prop-2-en-1- one |
| 192 | 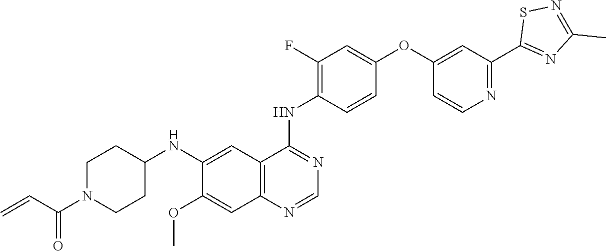 [SMILES: C=CC(=O)N1CCC(Nc2cc3c(Nc4ccc(Oc5ccnc(-c6nc(C)ns6)c5)cc4F)ncnc3cc2OC)CC1] | 1-(4-((4-((2-fluoro- 4-((2-(3-methyl- 1,2,4-thiadiazol- 5-yl)pyridin-4- yl)oxy)phenyl)amino)-7- methoxyquinazolin-6- yl)amino)piperidin- 1-yl)prop-2-en-1- one |
| 193 | 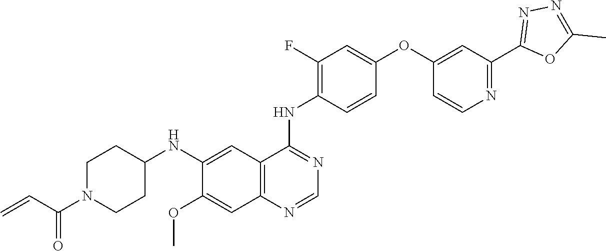 [SMILES: C=CC(=O)N1CCC(Nc2cc3c(Nc4ccc(Oc5ccnc(-c6nnc(C)o6)c5)cc4F)ncnc3cc2OC)CC1] | 1-(4-((4-((2-fluoro- 4-((2-(5-methyl- 1,3,4-oxadiazol- 2-yl)pyridin-4- yl)oxy)phenyl)amino)-7- methoxyquinazolin-6- yl)amino)piperidin- 1-yl)prop-2-en-1- one |
| 194 | 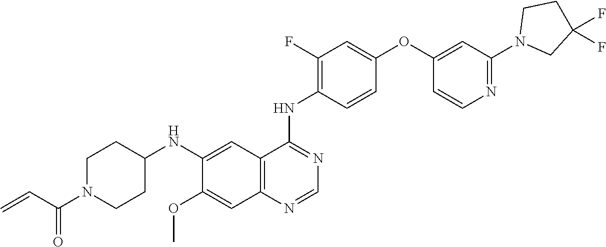 [SMILES: C=CC(=O)N1CCC(Nc2cc3c(Nc4ccc(Oc5ccnc(N6CCC(F)(F)C6)c5)cc4F)ncnc3cc2OC)CC1] | 1-(4-((4-((4-((2-(3,3- difluoropyrrolidin- 1-yl)pyridin-4- yl)oxy)-2-fluorophenyl) amino)-7- methoxyquinazolin-6- yl)amino)piperidin- 1-yl)prop-2-en-1- one |
| 195 | 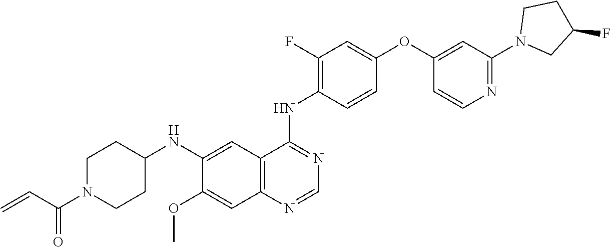 [SMILES: C=CC(=O)N1CCC(Nc2cc3c(Nc4ccc(Oc5ccnc(N6CC[C@@H](F)C6)c5)cc4F)ncnc3cc2OC)CC1] | (R)-1-(4-((4-((2- fluoro-4-((2-(3- fluoropyrrolidin- 1-yl)pyridin-4- yl)oxy)phenyl)amino)-7- methoxyquinazolin-6- yl)amino)piperidin- 1-yl)prop-2-en-1- one |
| 196 | 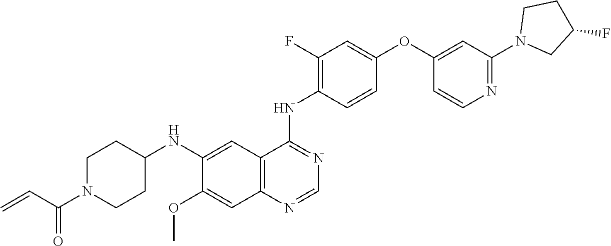 [SMILES: C=CC(=O)N1CCC(Nc2cc3c(Nc4ccc(Oc5ccnc(N6CC[C@H](F)C6)c5)cc4F)ncnc3cc2OC)CC1] | (S)-1-(4-((4-((2- fluoro-4-((2-(3- fluoropyrrolidin- 1-yl)pyridin-4- yl)oxy)phenyl)amino)-7- methoxyquinazolin-6- yl)amino)piperidin- 1-yl)prop-2-en-1- one |
| 197 | 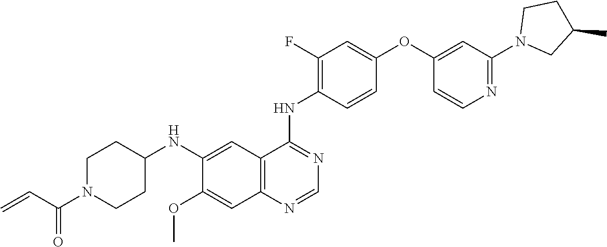 [SMILES: C=CC(=O)N1CCC(Nc2cc3c(Nc4ccc(Oc5ccnc(N6CC[C@@H](C)C6)c5)cc4F)ncnc3cc2OC)CC1] | (R)-1-(4-((4-((2- fluoro-4-((2-(3- methylpyrrolidin- 1-yl)pyridin-4- yl)oxy)phenyl)amino)-7- methoxyquinazolin-6- yl)amino)piperidin- 1-yl)prop-2-en-1- one |
| 198 | 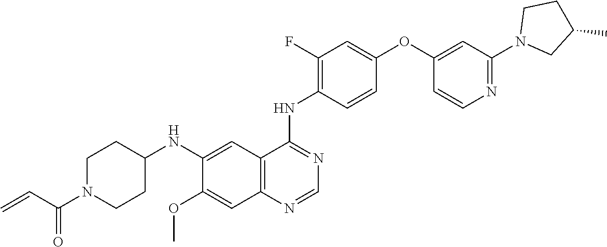 [SMILES: C=CC(=O)N1CCC(Nc2cc3c(Nc4ccc(Oc5ccnc(N6CC[C@H](C)C6)c5)cc4F)ncnc3cc2OC)CC1] | (S)-1-(4-((4-((2- fluoro-4-((2-(3- methylpyrrolidin- 1-yl)pyridin-4- yl)oxy)phenyl)amino)-7- methoxyquinazolin-6- yl)amino)piperidin- 1-yl)prop-2-en-1- one |
| 199 | 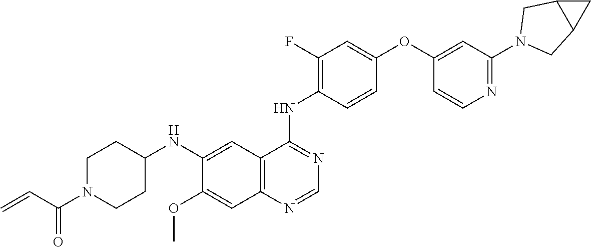 [SMILES: C=CC(=O)N1CCC(Nc2cc3c(Nc4ccc(Oc5ccnc(N6CC7CC7C6)c5)cc4F)ncnc3cc2OC)CC1] | 1-(4-((4-((4-((2-(3- azabicyclo[3.1.0] hexan-3-yl)pyridin- 4-yl)oxy)-2-fluorophenyl) amino)-7- methoxyquinazolin-6- yl)amino)piperidin- 1-yl)prop-2-en-1- one |
| 200 | 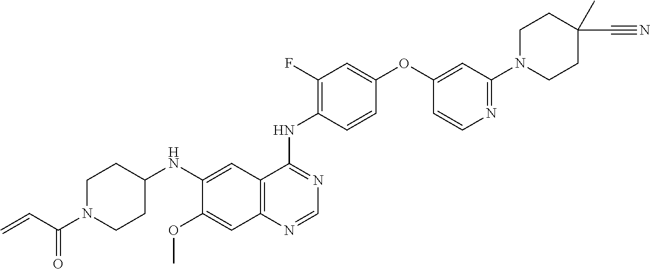 [SMILES: C=CC(=O)N1CCC(Nc2cc3c(Nc4ccc(Oc5ccnc(N6CCC(C)(C#N)CC6)c5)cc4F)ncnc3cc2OC)CC1] | 1-(4-(4-((6-((1- acryloylpiperidin-4- yl)amino)-7- methoxyquinazolin-4- yl)amino)-3- fluorophenoxy)pyridin-2- yl)-4-methylpiperidine- 4-carbonitrile |
| 201 | 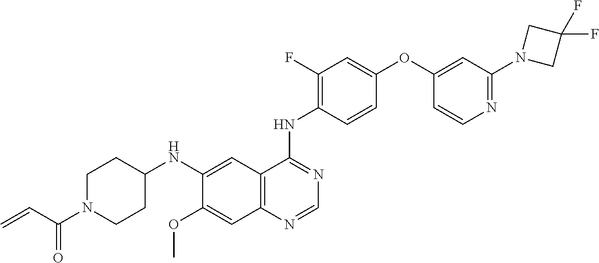 [SMILES: C=CC(=O)N1CCC(Nc2cc3c(Nc4ccc(Oc5ccnc(N6CC(F)(F)C6)c5)cc4F)ncnc3cc2OC)CC1] | 1-(4-((4-((4-((2- (3,3-difluoroazetidin- 1-yl)pyridin-4-yl)oxy)-2- fluorophenyl)amino)-7- methoxyquinazolin-6- yl)amino)piperidin- 1-yl)prop-2-en-1- one |
| 202 | 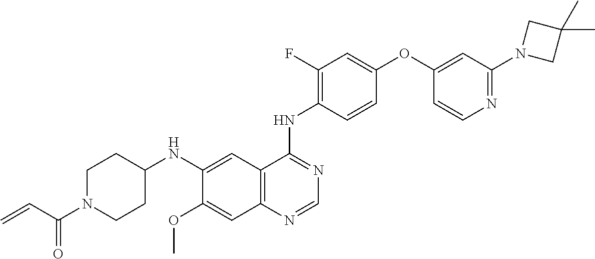 [SMILES: C=CC(=O)N1CCC(Nc2cc3c(Nc4ccc(Oc5ccnc(N6CC(C)(C)C6)c5)cc4F)ncnc3cc2OC)CC1] | 1-(4-((4-((4-((2-(3,3- dimethylazetidin- 1-yl)pyridin-4- yl)oxy)-2- fluorophenyl)amino)-7- methoxyquinazolin-6- yl)amino)piperidin- 1-yl)prop-2-en-1- one |
| 203 | 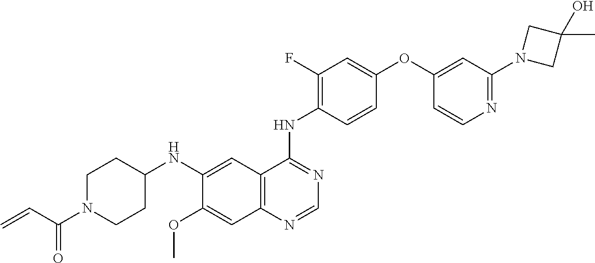 [SMILES: C=CC(=O)N1CCC(Nc2cc3c(Nc4ccc(Oc5ccnc(N6CC(C)(O)C6)c5)cc4F)ncnc3cc2OC)CC1] | 1-(4-((4-((2-fluoro- 4-((2-(3-hydroxy- 3-methylazetidin- 1-yl)pyridin-4- yl)oxy)phenyl)amino)-7- methoxyquinazolin-6- yl)amino)piperidin- 1-yl)prop-2-en-1- one |
| 204 | 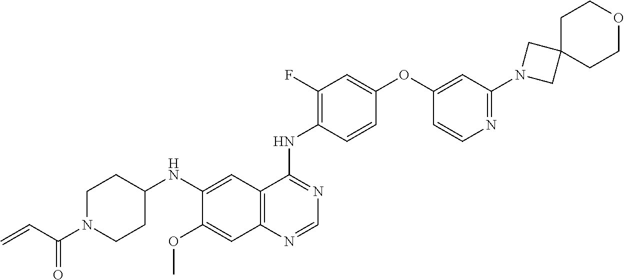 [SMILES: C=CC(=O)N1CCC(Nc2cc3c(Nc4ccc(Oc5ccnc(N6CC7(CCOCC7)C6)c5)cc4F)ncnc3cc2OC)CC1] | 1-(4-((4-((4-((2-(7-oxa-2- azaspiro[3.5]nonan- 2-yl)pyridin-4- yl)oxy)-2-fluorophenyl) amino)-7- methoxyquinazolin-6- yl)amino)piperidin- 1-yl)prop-2-en-1- one |
| 205 | 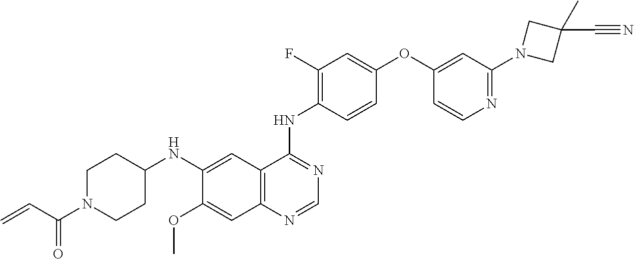 [SMILES: C=CC(=O)N1CCC(Nc2cc3c(Nc4ccc(Oc5ccnc(N6CC(C)(C#N)C6)c5)cc4F)ncnc3cc2OC)CC1] | 1-(4-(4-((6-((1- acryloylpiperidin-4- yl)amino)-7- methoxyquinazolin-4- yl)amino)-3- fluorophenoxy)pyridin-2- yl)-3-methylazetidine- 3-carbonitrile |
| 206 | 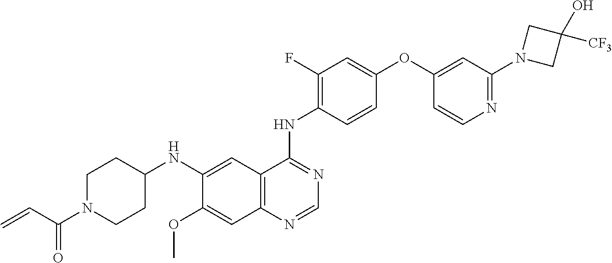 [SMILES: C=CC(=O)N1CCC(Nc2cc3c(Nc4ccc(Oc5ccnc(N6CC(O)(C(F)(F)F)C6)c5)cc4F)ncnc3cc2OC)CC1] | 1-(4-((4-((2-fluoro- 4-((2-(3-hydroxy- 3-(trifluoromethyl) azetidin-1- yl)pyridin-4-yl)oxy) phenyl)amino)-7- methoxyquinazolin-6- yl)amino)piperidin- 1-yl)prop-2-en-1- one |
| 207 | 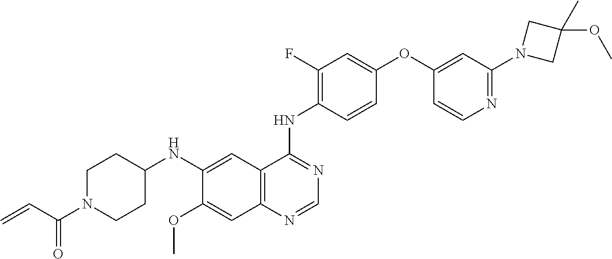 [SMILES: C=CC(=O)N1CCC(Nc2cc3c(Nc4ccc(Oc5ccnc(N6CC(C)(OC)C6)c5)cc4F)ncnc3cc2OC)CC1] | 1-(4-((4-((2-fluoro- 4-((2-(3-methoxy- 3-methylazetidin- 1-yl)pyridin-4- yl)oxy)phenyl)amino)-7- methoxyquinazolin-6- yl)amino)piperidin- 1-yl)prop-2-en-1- one |
| 208 | 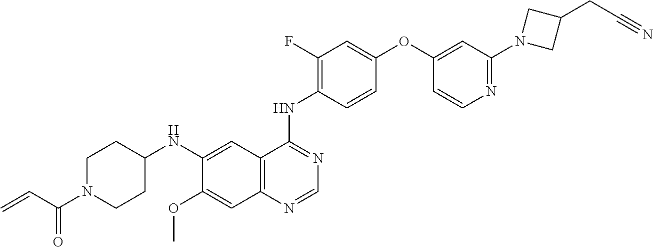 [SMILES: C=CC(=O)N1CCC(Nc2cc3c(Nc4ccc(Oc5ccnc(N6CC(CC#N)C6)c5)cc4F)ncnc3cc2OC)CC1] | 2-(1-(4-(4-((6-((1- acryloylpiperidin-4- yl)amino)-7- methoxyquinazolin-4- yl)amino)-3- fluorophenoxy)pyridin-2- yl)azetidin-3-yl)acetonitrile |
| 209 | 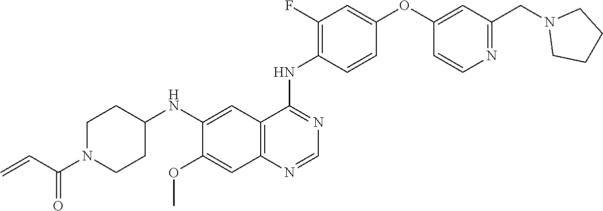 [SMILES: C=CC(=O)N1CCC(Nc2cc3c(Nc4ccc(Oc5ccnc(CN6CCCC6)c5)cc4F)ncnc3cc2OC)CC1] | 1-(4-((4-((2-fluoro- 4-((2-(pyrrolidin-1- ylmethyl)pyridin-4- yl)oxy)phenyl)amino)-7- methoxyquinazolin-6- yl)amino)piperidin- 1-yl)prop-2-en-1- one |
| 210 | 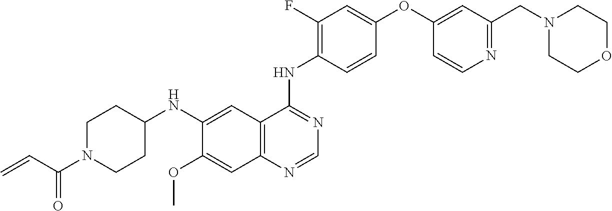 [SMILES: C=CC(=O)N1CCC(Nc2cc3c(Nc4ccc(Oc5ccnc(CN6CCOCC6)c5)cc4F)ncnc3cc2OC)CC1] | 1-(4-((4-((2-fluoro-4-((2- (morpholinomethyl) pyridin-4- yl)oxy)phenyl)amino)-7- methoxyquinazolin-6- yl)amino)piperidin- 1-yl)prop-2-en-1- one |
| 211 | 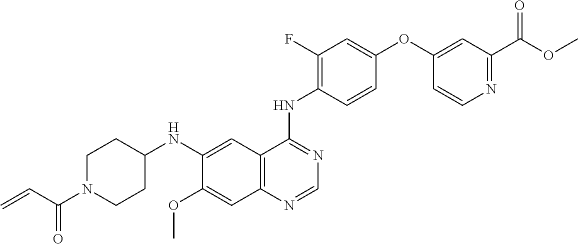 [SMILES: C=CC(=O)N1CCC(Nc2cc3c(Nc4ccc(Oc5ccnc(C(=O)OC)c5)cc4F)ncnc3cc2OC)CC1] | methyl 4-(4-((6-((1- acryloylpiperidin- 4-yl)amino)-7- methoxyquinazolin-4- yl)amino)-3-fluorophenoxy) picolinate |
| 212 | 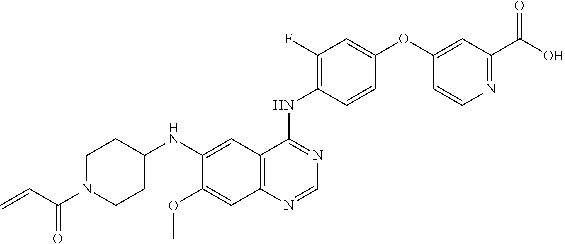 [SMILES: C=CC(=O)N1CCC(Nc2cc3c(Nc4ccc(Oc5ccnc(C(=O)O)c5)cc4F)ncnc3cc2OC)CC1] | 4-(4-((6-((1- acryloylpiperidin-4- yl)amino)-7- methoxyquinazolin-4- yl)amino)-3-fluorophenoxy) picolinic acid |
| 213 | 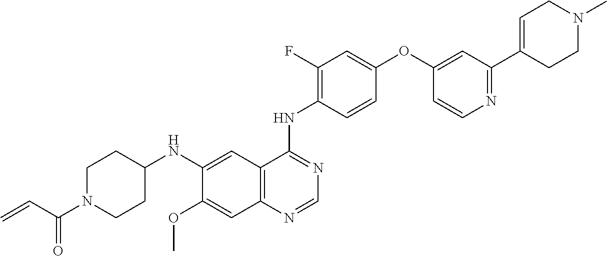 [SMILES: C=CC(=O)N1CCC(Nc2cc3c(Nc4ccc(Oc5ccnc(C6=CCN(C)CC6)c5)cc4F)ncnc3cc2OC)CC1] | 1-(4-((4-((2-fluoro- 4-((1′-methyl- 1′,2′,3′,6′-tetrahydro- [2,4′-bipyridin]- 4-yl)oxy)phenyl)amino)-7- methoxyquinazolin-6- yl)amino)piperidin- 1-yl)prop-2-en-1- one |
| 214 | 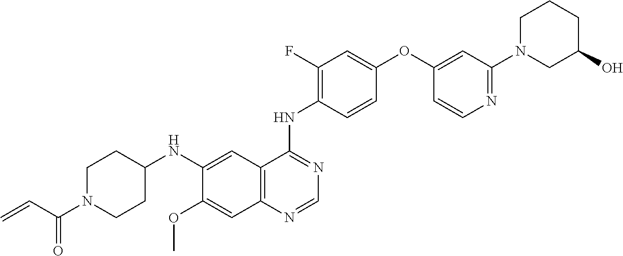 [SMILES: C=CC(=O)N1CCC(Nc2cc3c(Nc4ccc(Oc5ccnc(N6CCC[C@@H](O)C6)c5)cc4F)ncnc3cc2OC)CC1] | (R)-1-(4-((4-((2- fluoro-4-((2-(3- hydroxypiperidin- 1-yl)pyridin-4- yl)oxy)phenyl)amino)-7- methoxyquinazolin-6- yl)amino)piperidin- 1-yl)prop-2-en-1- one |
| 215 | 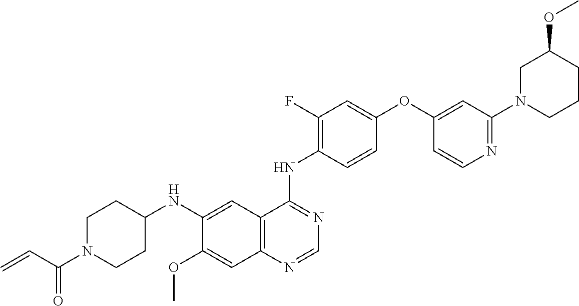 [SMILES: C=CC(=O)N1CCC(Nc2cc3c(Nc4ccc(Oc5ccnc(N6CCC[C@H](OC)C6)c5)cc4F)ncnc3cc2OC)CC1] | (S)-1-(4-((4-((2- fluoro-4-((2-(3- methoxypiperidin- 1-yl)pyridin-4- yl)oxy)phenyl)amino)-7- methoxyquinazolin-6- yl)amino)piperidin- 1-yl)prop-2-en-1- one |
| 216 | 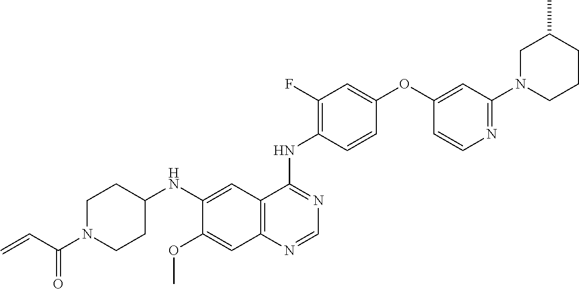 [SMILES: C=CC(=O)N1CCC(Nc2cc3c(Nc4ccc(Oc5ccnc(N6CCC[C@@H](C)C6)c5)cc4F)ncnc3cc2OC)CC1] | (R)-1-(4-((4-((2- fluoro-4-((2-(3- methylpiperidin- 1-yl)pyridin-4- yl)oxy)phenyl)amino)-7- methoxyquinazolin-6- yl)amino)piperidin- 1-yl)prop-2-en-1- one |
| 217 | 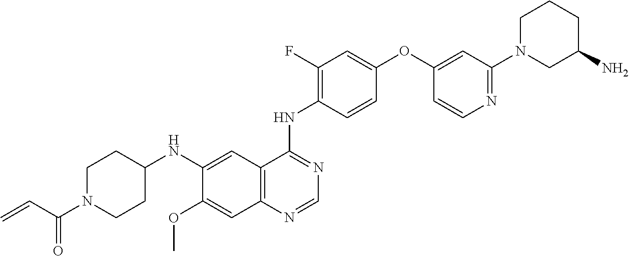 [SMILES: C=CC(=O)N1CCC(Nc2cc3c(Nc4ccc(Oc5ccnc(N6CCC[C@@H](N)C6)c5)cc4F)ncnc3cc2OC)CC1] | (R)-1-(4-((4-((4-((2-(3- aminopiperidin- 1-yl)pyridin-4- yl)oxy)-2- fluorophenyl)amino)-7- methoxyquinazolin-6- yl)amino)piperidin- 1-yl)prop-2-en-1- one |
| 218 | 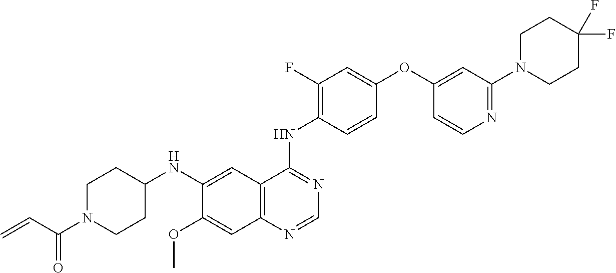 [SMILES: C=CC(=O)N1CCC(Nc2cc3c(Nc4ccc(Oc5ccnc(N6CCC(F)(F)CC6)c5)cc4F)ncnc3cc2OC)CC1] | 1-(4-((4-((4-((2-(4,4- difluoropiperidin- 1-yl)pyridin- 4-yl)oxy)-2- fluorophenyl)amino)-7- methoxyquinazolin-6- yl)amino)piperidin- 1-yl)prop-2-en-1- one |
| 219 | 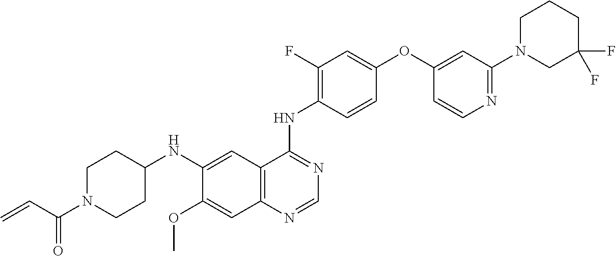 [SMILES: C=CC(=O)N1CCC(Nc2cc3c(Nc4ccc(Oc5ccnc(N6CCCC(F)(F)C6)c5)cc4F)ncnc3cc2OC)CC1] | 1-(4-((4-((4-((2-(3,3- difluoropiperidin- 1-yl)pyridin-4- yl)oxy)-2- fluorophenyl)amino)-7- methoxyquinazolin-6- yl)amino)piperidin- 1-yl)prop-2-en-1- one |
| 220 | 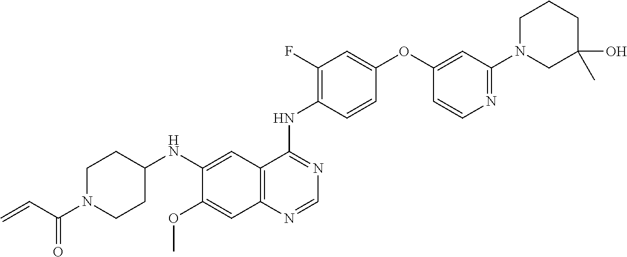 [SMILES: C=CC(=O)N1CCC(Nc2cc3c(Nc4ccc(Oc5ccnc(N6CCCC(C)(O)C6)c5)cc4F)ncnc3cc2OC)CC1] | 1-(4-((4-((2-fluoro- 4-((2-(3-hydroxy- 3-methylpiperidin- 1-yl)pyridin-4- yl)oxy)phenyl)amino)-7- methoxyquinazolin-6- yl)amino)piperidin- 1-yl)prop-2-en-1- one |
| 221 | 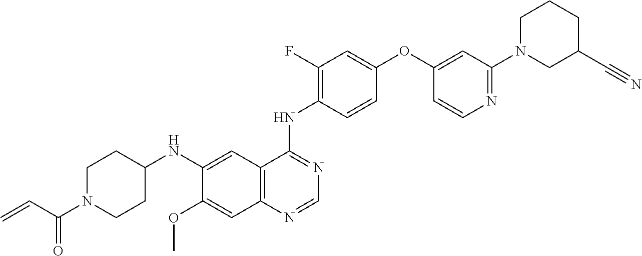 [SMILES: C=CC(=O)N1CCC(Nc2cc3c(Nc4ccc(Oc5ccnc(N6CCCC(C#N)C6)c5)cc4F)ncnc3cc2OC)CC1] | 1-(4-(4-((6-((1- acryloylpiperidin-4- yl)amino)-7- methoxyquinazolin-4- yl)amino)-3-fluorophenoxy) pyridin-2- yl)piperidine-3-carbonitrile |
| 222 | 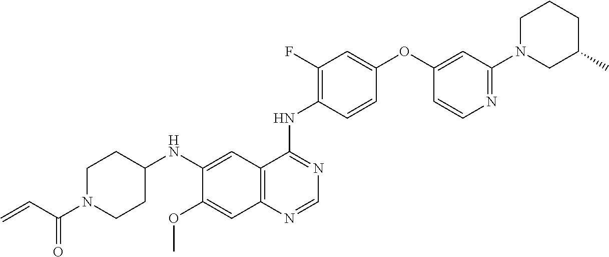 [SMILES: C=CC(=O)N1CCC(Nc2cc3c(Nc4ccc(Oc5ccnc(N6CCC[C@H](C)C6)c5)cc4F)ncnc3cc2OC)CC1] | (S)-1-(4-((4-((2- fluoro-4-((2-(3- methylpiperidin- 1-yl)pyridin-4- yl)oxy)phenyl)amino)-7- methoxyquinazolin-6- yl)amino)piperidin- 1-yl)prop-2-en-1- one |
| 223 | 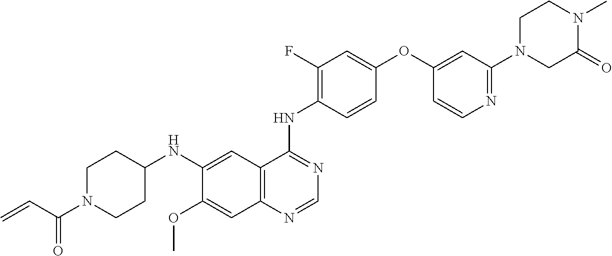 [SMILES: C=CC(=O)N1CCC(Nc2cc3c(Nc4ccc(Oc5ccnc(N6CCN(C)C(=O)C6)c5)cc4F)ncnc3cc2OC)CC1] | 4-(4-(4-((6-((1- acryloylpiperidin-4- yl)amino)-7- methoxyquinazolin-4- yl)amino)-3-fluorophenoxy) pyridin-2- yl)-1-methylpiperazin- 2-one |
| 224 | 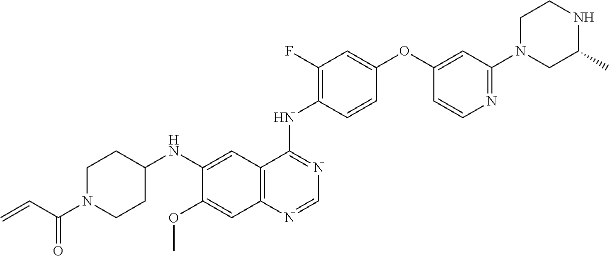 [SMILES: C=CC(=O)N1CCC(Nc2cc3c(Nc4ccc(Oc5ccnc(N6CCN[C@H](C)C6)c5)cc4F)ncnc3cc2OC)CC1] | (R)-1-(4-((4-((2- fluoro-4-((2-(3- methylpiperazin- 1-yl)pyridin-4- yl)oxy)phenyl)amino)-7- methoxyquinazolin-6- yl)amino)piperidin- 1-yl)prop-2-en-1- one |
| 225 | 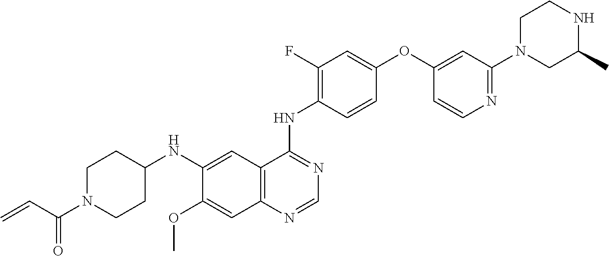 [SMILES: C=CC(=O)N1CCC(Nc2cc3c(Nc4ccc(Oc5ccnc(N6CCN[C@@H](C)C6)c5)cc4F)ncnc3cc2OC)CC1] | (S)-1-(4-((4-((2- fluoro-4-((2-(3- methylpiperazin- 1-yl)pyridin-4- yl)oxy)phenyl)amino)-7- methoxyquinazolin-6- yl)amino)piperidin- 1-yl)prop-2-en-1- one |
| 226 | 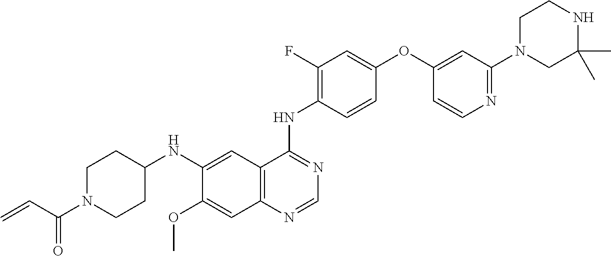 [SMILES: C=CC(=O)N1CCC(Nc2cc3c(Nc4ccc(Oc5ccnc(N6CCNC(C)(C)C6)c5)cc4F)ncnc3cc2OC)CC1] | 1-(4-((4-((4-((2-(3,3- dimethylpiperazin- 1-yl)pyridin- 4-yl)oxy)-2- fluorophenyl)amino)-7- methoxyquinazolin-6- yl)amino)piperidin- 1-yl)prop-2-en-1- one |
| 227 | 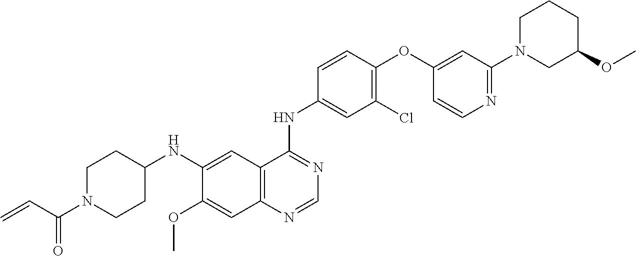 [SMILES: C=CC(=O)N1CCC(Nc2cc3c(Nc4ccc(Oc5ccnc(N6CCC[C@@H](OC)C6)c5)c(Cl)c4)ncnc3cc2OC)CC1] | (R)-1-(4-((4-((3- chloro-4-((2-(3- methoxypiperidin- 1-yl)pyridin-4- yl)oxy)phenyl)amino)-7- methoxyquinazolin-6- yl)amino)piperidin- 1-yl)prop-2-en-1- one |
| 228 | 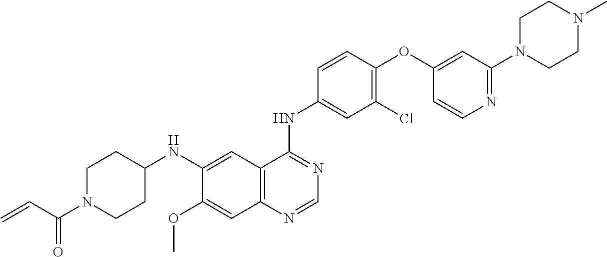 [SMILES: C=CC(=O)N1CCC(Nc2cc3c(Nc4ccc(Oc5ccnc(N6CCN(C)CC6)c5)c(Cl)c4)ncnc3cc2OC)CC1] | 1-(4-((4-((3-chloro-4-((2-(4- methylpiperazin- 1-yl)pyridin-4- yl)oxy)phenyl)amino)-7- methoxyquinazolin-6- yl)amino)piperidin- 1-yl)prop-2-en-1- one |
| 229 | 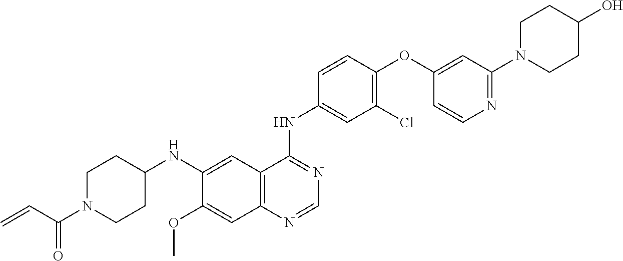 [SMILES: C=CC(=O)N1CCC(Nc2cc3c(Nc4ccc(Oc5ccnc(N6CCC(O)CC6)c5)c(Cl)c4)ncnc3cc2OC)CC1] | 1-(4-((4-((3-chloro-4-((2-(4- hydroxypiperidin- 1-yl)pyridin-4- yl)oxy)phenyl)amino)-7- methoxyquinazolin-6- yl)amino)piperidin- 1-yl)prop-2-en-1- one |
| 230 | 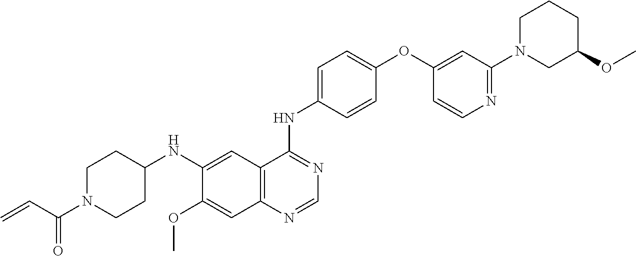 [SMILES: C=CC(=O)N1CCC(Nc2cc3c(Nc4ccc(Oc5ccnc(N6CCC[C@@H](OC)C6)c5)cc4)ncnc3cc2OC)CC1] | (R)-1-(4-((7-methoxy- 4-((4-((2-(3- methoxypiperidin- 1-yl)pyridin-4- yl)oxy)phenyl)amino) quinazolin-6- yl)amino)piperidin- 1-yl)prop-2-en-1- one |
| 231 | 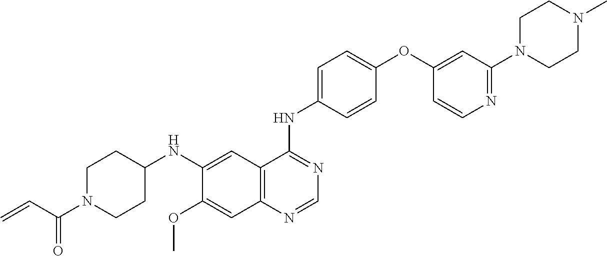 [SMILES: C=CC(=O)N1CCC(Nc2cc3c(Nc4ccc(Oc5ccnc(N6CCN(C)CC6)c5)cc4)ncnc3cc2OC)CC1] | 1-(4-((7-methoxy- 4-((4-((2-(4- methylpiperazin- 1-yl)pyridin-4- yl)oxy)phenyl)amino) quinazolin-6- yl)amino)piperidin- 1-yl)prop-2-en-1- one |
| 232 | 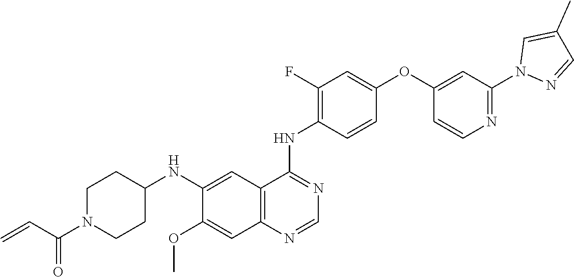 [SMILES: C=CC(=O)N1CCC(Nc2cc3c(Nc4ccc(Oc5ccnc(-n6cc(C)cn6)c5)cc4F)ncnc3cc2OC)CC1] | 1-(4-((4-((2-fluoro- 4-((2-(4-methyl- 1H-pyrazol-1-yl)pyridin-4- yl)oxy)phenyl)amino)-7- methoxyquinazolin-6- yl)amino)piperidin- 1-yl)prop-2-en-1- one |
| 233 | 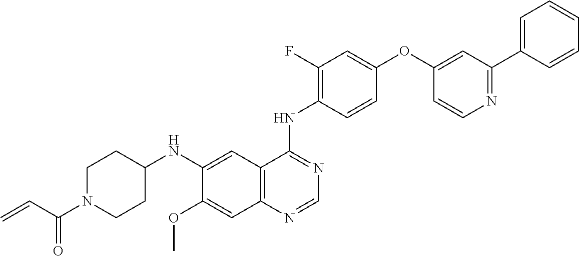 [SMILES: C=CC(=O)N1CCC(Nc2cc3c(Nc4ccc(Oc5ccnc(-c6ccccc6)c5)cc4F)ncnc3cc2OC)CC1] | 1-(4-((4-((2-fluoro-4-((2- phenylpyridin-4- yl)oxy)phenyl)amino)-7- methoxyquinazolin-6- yl)amino)piperidin- 1-yl)prop-2-en-1- one |
| 234 | 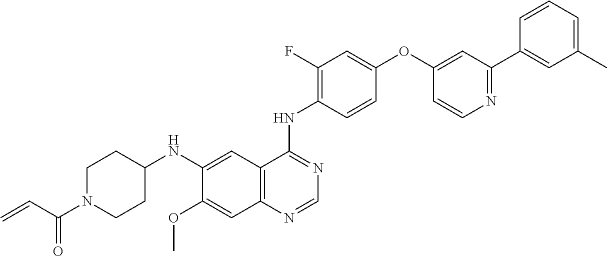 [SMILES: C=CC(=O)N1CCC(Nc2cc3c(Nc4ccc(Oc5ccnc(-c6cccc(C)c6)c5)cc4F)ncnc3cc2OC)CC1] | 1-(4-((4-((2-fluoro-4-((2-(m- tolyl)pyridin-4-yl) oxy)phenyl)amino)- 7-methoxyquinazolin-6- yl)amino)piperidin- 1-yl)prop-2-en-1- one |
| 235 | 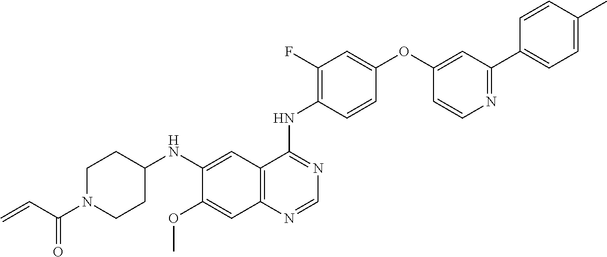 [SMILES: C=CC(=O)N1CCC(Nc2cc3c(Nc4ccc(Oc5ccnc(-c6ccc(C)cc6)c5)cc4F)ncnc3cc2OC)CC1] | 1-(4-((4-((2-fluoro-4-((2-(p- tolyl)pyridin-4-yl)oxy) phenyl)amino)- 7-methoxyquinazolin-6- yl)amino)piperidin- 1-yl)prop-2-en-1- one |
| 236 | 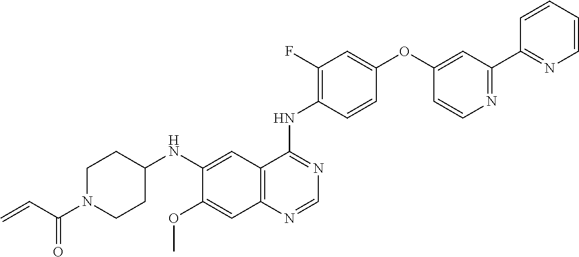 [SMILES: C=CC(=O)N1CCC(Nc2cc3c(Nc4ccc(Oc5ccnc(-c6ccccn6)c5)cc4F)ncnc3cc2OC)CC1] | 1-(4-((4-((4-([2,2′- bipyridin]-4-yloxy)- 2-fluorophenyl)amino)-7- methoxyquinazolin-6- yl)amino)piperidin- 1-yl)prop-2-en-1- one |
| 237 | 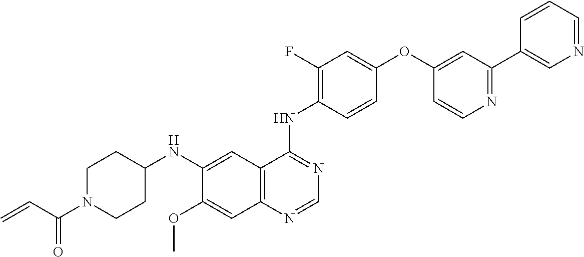 [SMILES: C=CC(=O)N1CCC(Nc2cc3c(Nc4ccc(Oc5ccnc(-c6cccnc6)c5)cc4F)ncnc3cc2OC)CC1] | 1-(4-((4-((4-([2,3′- bipyridin]-4-yloxy)- 2-fluorophenyl)amino)-7- methoxyquinazolin-6- yl)amino)piperidin- 1-yl)prop-2-en-1- one |
| 238 | 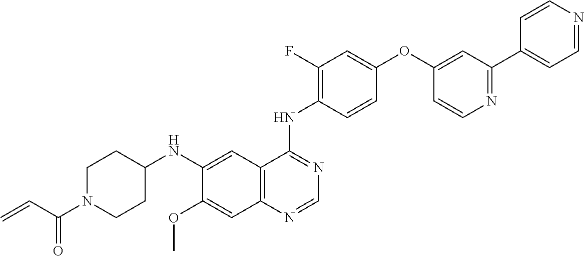 [SMILES: C=CC(=O)N1CCC(Nc2cc3c(Nc4ccc(Oc5ccnc(-c6ccncc6)c5)cc4F)ncnc3cc2OC)CC1] | 1-(4-((4-((4-([2,4′- bipyridin]-4-yloxy)- 2-fluorophenyl)amino)-7- methoxyquinazolin-6- yl)amino)piperidin- 1-yl)prop-2-en-1- one |
| 239 | 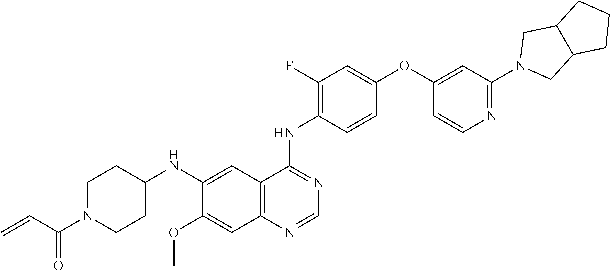 [SMILES: C=CC(=O)N1CCC(Nc2cc3c(Nc4ccc(Oc5ccnc(N6CC7CCCC7C6)c5)cc4F)ncnc3cc2OC)CC1] | 1-(4-((4-((2-fluoro-4-((2- (hexahydrocyclopenta [c]pyrrol- 2(1H)-yl)pyridin-4- yl)oxy)phenyl)amino)-7- methoxyquinazolin-6- yl)amino)piperidin- 1-yl)prop-2-en-1- one |
| 240 | 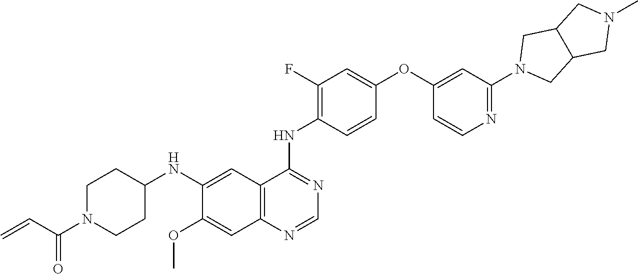 [SMILES: C=CC(=O)N1CCC(Nc2cc3c(Nc4ccc(Oc5ccnc(N6CC7CN(C)CC7C6)c5)cc4F)ncnc3cc2OC)CC1] | 1-(4-((4-((2-fluoro-4-((2-(5- methylhexahydropyrrolo[3,4- c]pyrrol-2(1H)-yl)pyridin-4- yl)oxy)phenyl)amino)-7- methoxyquinazolin-6- yl)amino)piperidin- 1-yl)prop-2-en-1- one |
| 241 | 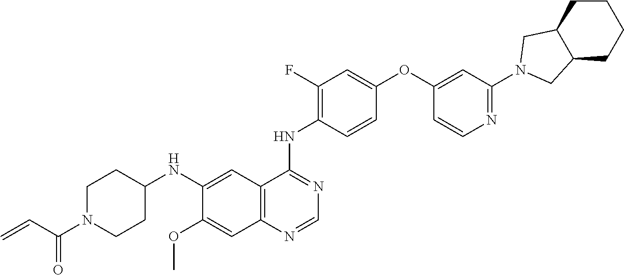 [SMILES: C=CC(=O)N1CCC(Nc2cc3c(Nc4ccc(Oc5ccnc(N6C[C@H]7CCCC[C@H]7C6)c5)cc4F)ncnc3cc2OC)CC1] | 1-(4-((4-((2-fluoro- 4-((2-((3aR,7aS)- octahydro-2H-isoindol- 2-yl)pyridin-4- yl)oxy)phenyl)amino)-7- methoxyquinazolin-6- yl)amino)piperidin- 1-yl)prop-2-en-1- one |
| 242 | 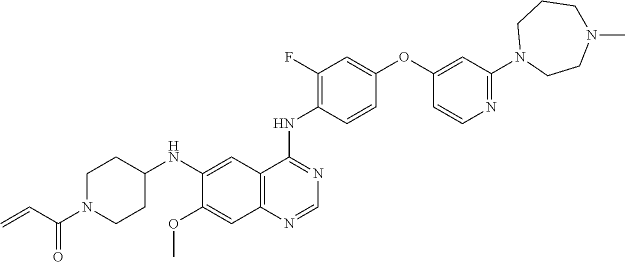 [SMILES: C=CC(=O)N1CCC(Nc2cc3c(Nc4ccc(Oc5ccnc(N6CCCN(C)CC6)c5)cc4F)ncnc3cc2OC)CC1] | 1-(4-((4-((2-fluoro- 4-((2-(4-methyl- 1,4-diazepan-1-yl)pyridin-4- yl)oxy)phenyl)amino)-7- methoxyquinazolin-6- yl)amino)piperidin- 1-yl)prop-2-en-1- one |
| 243 | 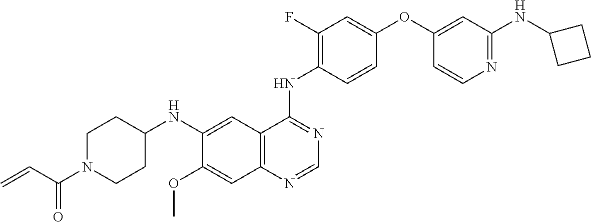 [SMILES: C=CC(=O)N1CCC(Nc2cc3c(Nc4ccc(Oc5ccnc(NC6CCC6)c5)cc4F)ncnc3cc2OC)CC1] | 1-(4-((4-((4-((2- (cyclobutylamino) pyridin-4-yl)oxy)-2- fluorophenyl)amino)-7- methoxyquinazolin-6- yl)amino)piperidin- 1-yl)prop-2-en-1- one |
| 244 | 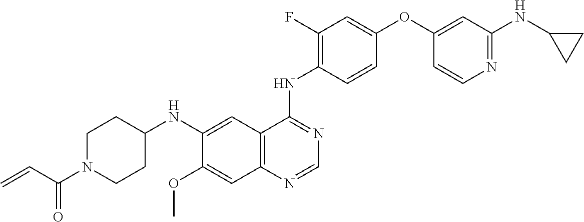 [SMILES: C=CC(=O)N1CCC(Nc2cc3c(Nc4ccc(Oc5ccnc(NC6CC6)c5)cc4F)ncnc3cc2OC)CC1] | 1-(4-((4-((4-((2- (cyclopropylamino) pyridin-4-yl)oxy)- 2-fluorophenyl)amino)-7- methoxyquinazolin-6- yl)amino)piperidin- 1-yl)prop-2-en-1- one |
| 245 | 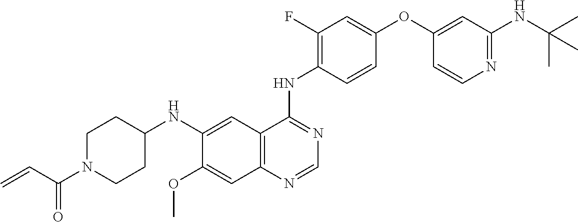 [SMILES: C=CC(=O)N1CCC(Nc2cc3c(Nc4ccc(Oc5ccnc(NC(C)(C)C)c5)cc4F)ncnc3cc2OC)CC1] | 1-(4-((4-((4-((2-(tert- butylamino)pyridin- 4-yl)oxy)-2- fluorophenyl)amino)-7- methoxyquinazolin-6- yl)amino)piperidin- 1-yl)prop-2-en-1- one |
| 246 | 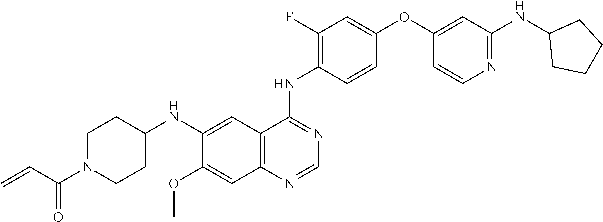 [SMILES: C=CC(=O)N1CCC(Nc2cc3c(Nc4ccc(Oc5ccnc(NC6CCCC6)c5)cc4F)ncnc3cc2OC)CC1] | 1-(4-((4-((4-((2- (cyclopentylamino) pyridin-4-yl)oxy)- 2-fluorophenyl)amino)-7- methoxyquinazolin-6- yl)amino)piperidin- 1-yl)prop-2-en-1- one |
| 247 | 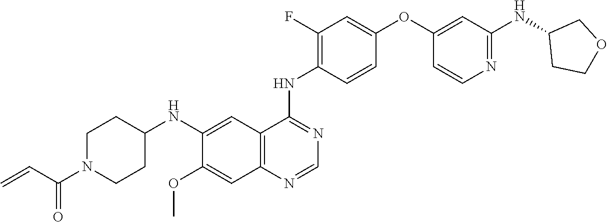 [SMILES: C=CC(=O)N1CCC(Nc2cc3c(Nc4ccc(Oc5ccnc(N[C@H]6CCOC6)c5)cc4F)ncnc3cc2OC)CC1] | (S)-1-(4-((4-((2-fluoro-4-((2- ((tetrahydrofuran-3- yl)amino)pyridin- 4-yl)oxy)phenyl)amino)-7- methoxyquinazolin-6- yl)amino)piperidin- 1-yl)prop-2-en-1- one |
| 248 | 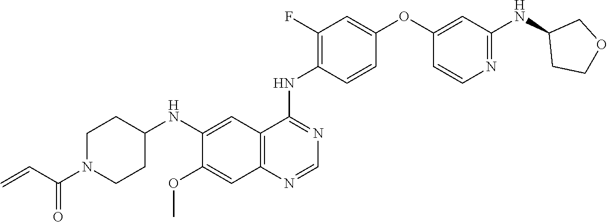 [SMILES: C=CC(=O)N1CCC(Nc2cc3c(Nc4ccc(Oc5ccnc(N[C@@H]6CCOC6)c5)cc4F)ncnc3cc2OC)CC1] | (R)-1-(4-((4-((2-fluoro-4-((2- ((tetrahydrofuran-3- yl)amino)pyridin- 4-yl)oxy)phenyl)amino)-7- methoxyquinazolin-6- yl)amino)piperidin- 1-yl)prop-2-en-1- one |
| 249 | 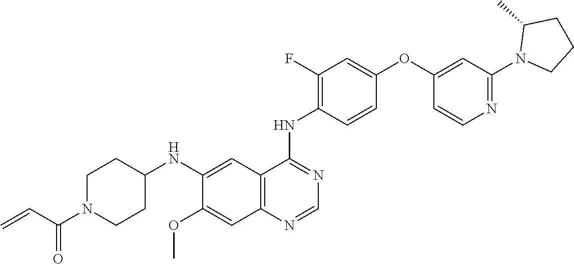 [SMILES: C=CC(=O)N1CCC(Nc2cc3c(Nc4ccc(Oc5ccnc(N6CCC[C@H]6C)c5)cc4F)ncnc3cc2OC)CC1] | (R)-1-(4-((4-((2- fluoro-4-((2-(2- methylpyrrolidin- 1-yl)pyridin-4- yl)oxy)phenyl)amino)-7- methoxyquinazolin-6- yl)amino)piperidin- 1-yl)prop-2-en-1- one |
| 250 | 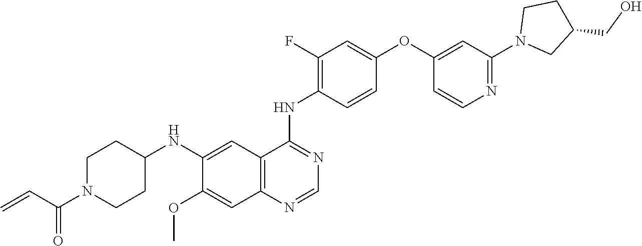 [SMILES: C=CC(=O)N1CCC(Nc2cc3c(Nc4ccc(Oc5ccnc(N6CC[C@H](CO)C6)c5)cc4F)ncnc3cc2OC)CC1] | (S)-1-(4-((4-((2- fluoro-4-((2-(3- (hydroxymethyl)pyrrolidin-1- yl)pyridin-4-yl)oxy) phenyl)amino)-7- methoxyquinazolin-6- yl)amino)piperidin- 1-yl)prop-2-en-1- one |
| 251 | 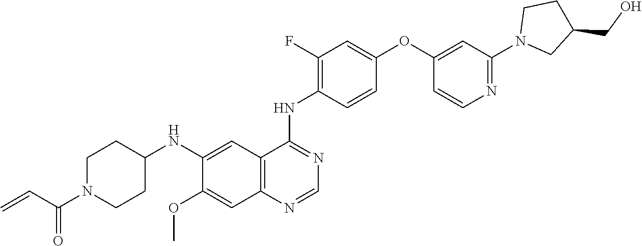 [SMILES: C=CC(=O)N1CCC(Nc2cc3c(Nc4ccc(Oc5ccnc(N6CC[C@@H](CO)C6)c5)cc4F)ncnc3cc2OC)CC1] | (R)-1-(4-((4- ((2-fluoro-4-((2-(3- (hydroxymethyl)pyrrolidin-1- yl)pyridin-4-yl)oxy) phenyl)amino)-7- methoxyquinazolin-6- yl)amino)piperidin- 1-yl)prop-2-en-1- one |
| 252 | 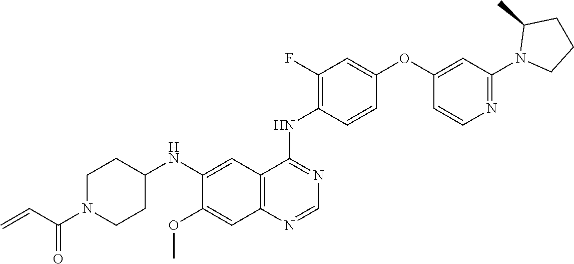 [SMILES: C=CC(=O)N1CCC(Nc2cc3c(Nc4ccc(Oc5ccnc(N6CCC[C@@H]6C)c5)cc4F)ncnc3cc2OC)CC1] | (S)-1-(4-((4-((2- fluoro-4-((2-(2- methylpyrrolidin- 1-yl)pyridin-4- yl)oxy)phenyl)amino)-7- methoxyquinazolin-6- yl)amino)piperidin- 1-yl)prop-2-en-1- one |
| 253 | 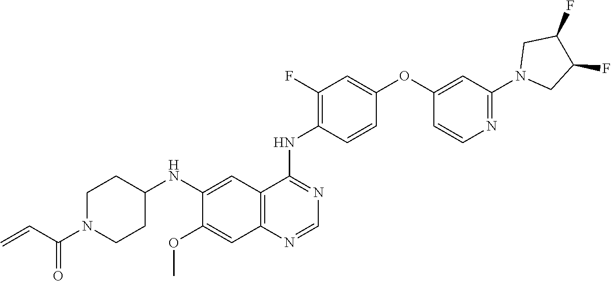 [SMILES: C=CC(=O)N1CCC(Nc2cc3c(Nc4ccc(Oc5ccnc(N6C[C@@H](F)[C@@H](F)C6)c5)cc4F)ncnc3cc2OC)CC1] | 1-(4-((4-((4-((2-((3S,4R)-3,4- difluoropyrrolidin- 1-yl)pyridin-4- yl)oxy)-2-fluorophenyl) amino)-7- methoxyquinazolin-6- yl)amino)piperidin- 1-yl)prop-2-en-1- one |
| 254 | 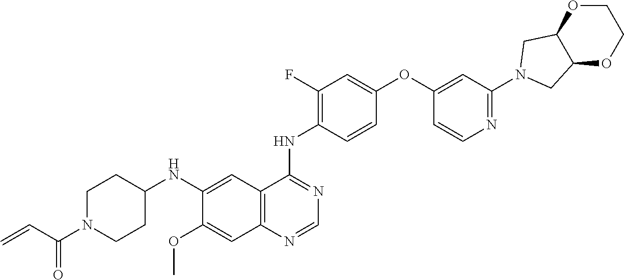 [SMILES: C=CC(=O)N1CCC(Nc2cc3c(Nc4ccc(Oc5ccnc(N6C[C@@H]7OCCO[C@@H]7C6)c5)cc4F)ncnc3cc2OC)CC1] | 1-(4-((4-((2-fluoro- 4-((2-((4aR,7aS)- hexahydro-6H- [1,4]dioxino[2,3- c]pyrrol-6-yl)pyridin-4- yl)oxy)phenyl)amino)-7- methoxyquinazolin-6- yl)amino)piperidin- 1-yl)prop-2-en-1- one |
| 255 | 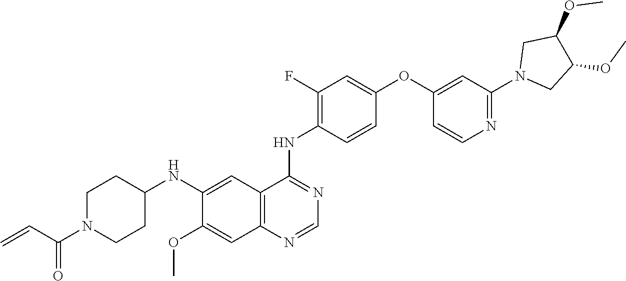 [SMILES: C=CC(=O)N1CCC(Nc2cc3c(Nc4ccc(Oc5ccnc(N6C[C@@H](OC)[C@H](OC)C6)c5)cc4F)ncnc3cc2OC)CC1] | 1-(4-((4-((4-((2-((3R,4R)-3,4- dimethoxypyrrolidin- 1-yl)pyridin-4- yl)oxy)-2-fluorophenyl) amino)-7- methoxyquinazolin-6- yl)amino)piperidin- 1-yl)prop-2-en-1- one |
| 256 | 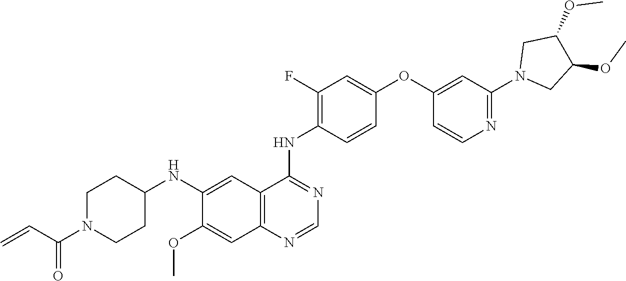 [SMILES: C=CC(=O)N1CCC(Nc2cc3c(Nc4ccc(Oc5ccnc(N6C[C@H](OC)[C@@H](OC)C6)c5)cc4F)ncnc3cc2OC)CC1] | 1-(4-((4-((4-((2-((3S,4S)-3,4- dimethoxypyrrolidin- 1-yl)pyridin-4- yl)oxy)-2- fluorophenyl)amino)-7- methoxyquinazolin-6- yl)amino)piperidin- 1-yl)prop-2-en-1- one |
| 257 | 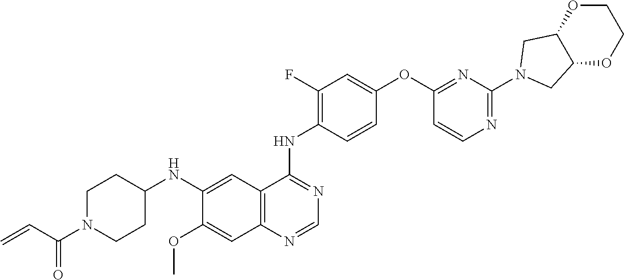 [SMILES: C=CC(=O)N1CCC(Nc2cc3c(Nc4ccc(Oc5ccnc(N6C[C@@H]7OCCO[C@@H]7C6)n5)cc4F)ncnc3cc2OC)CC1] | 1-(4-((4-((2-fluoro- 4-((2-((4aR,7aS)- hexahydro-6H-[1,4] dioxino[2,3- c]pyrrol-6-yl)pyrimidin-4- yl)oxy)phenyl)amino)-7- methoxyquinazolin-6- yl)amino)piperidin- 1-yl)prop-2-en-1- one |
| 258 | 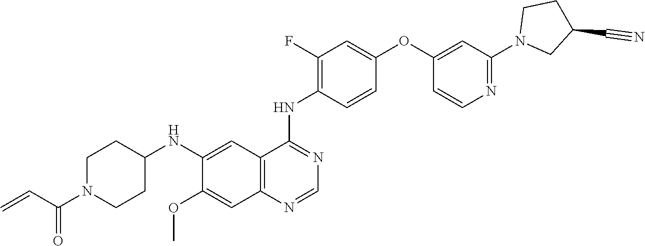 [SMILES: C=CC(=O)N1CCC(Nc2cc3c(Nc4ccc(Oc5ccnc(N6CC[C@@H](C#N)C6)c5)cc4F)ncnc3cc2OC)CC1] | (R)-1-(4-(4-((6-((1- acryloylpiperidin- 4-yl)amino)-7- methoxyquinazolin-4- yl)amino)-3-fluorophenoxy) pyridin-2- yl)pyrrolidine-3-carbonitrile |
| 259 | 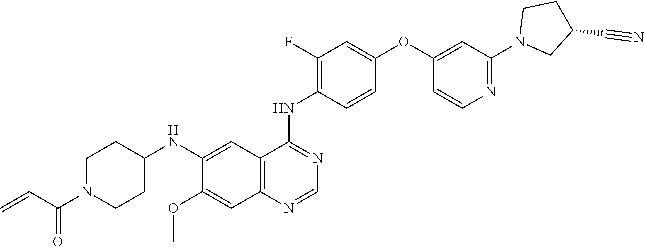 [SMILES: C=CC(=O)N1CCC(Nc2cc3c(Nc4ccc(Oc5ccnc(N6CC[C@H](C#N)C6)c5)cc4F)ncnc3cc2OC)CC1] | (S)-1-(4-(4-((6-((1- acryloylpiperidin- 4-yl)amino)-7- methoxyquinazolin-4- yl)amino)-3-fluorophenoxy) pyridin-2- yl)pyrrolidine-3-carbonitrile |
| 260 | 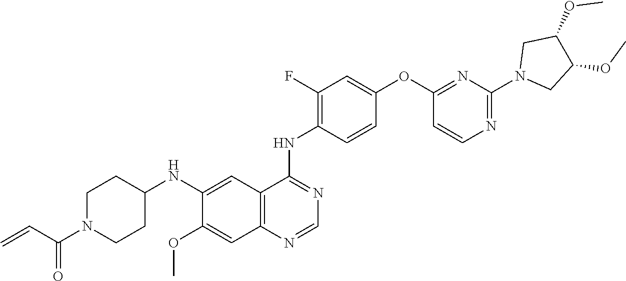 [SMILES: C=CC(=O)N1CCC(Nc2cc3c(Nc4ccc(Oc5ccnc(N6C[C@H](OC)[C@H](OC)C6)n5)cc4F)ncnc3cc2OC)CC1] | 1-(4-((4-((4-((2-((3R,4S)-3,4- dimethoxypyrrolidin- 1-yl)pyrimidin-4- yl)oxy)-2-fluorophenyl) amino)-7- methoxyquinazolin-6- yl)amino)piperidin- 1-yl)prop-2-en-1- one |
| 261 | 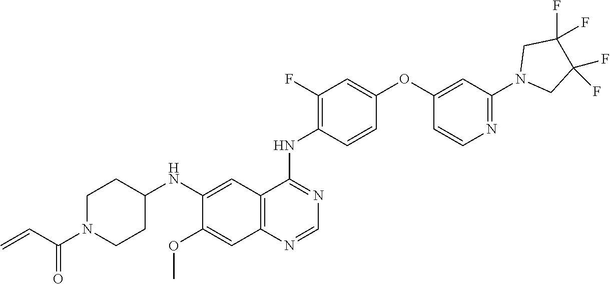 [SMILES: C=CC(=O)N1CCC(Nc2cc3c(Nc4ccc(Oc5ccnc(N6CC(F)(F)C(F)(F)C6)c5)cc4F)ncnc3cc2OC)CC1] | 1-(4-((4-((2-fluoro- 4-((2-(3,3,4,4- tetrafluoropyrrolidin- 1-yl)pyridin-4- yl)oxy)phenyl)amino)-7- methoxyquinazolin-6- yl)amino)piperidin- 1-yl)prop-2-en-1- one |
| 262 | 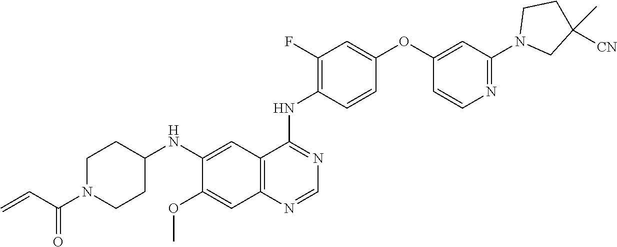 [SMILES: C=CC(=O)N1CCC(Nc2cc3c(Nc4ccc(Oc5ccnc(N6CCC(C)(C#N)C6)c5)cc4F)ncnc3cc2OC)CC1] | 1-(4-(4-((6-((1- acryloylpiperidin-4- yl)amino)-7- methoxyquinazolin-4- yl)amino)-3-fluorophenoxy) pyridin-2- yl)-3-methylpyrrolidine- 3-carbonitrile |
| 263 | 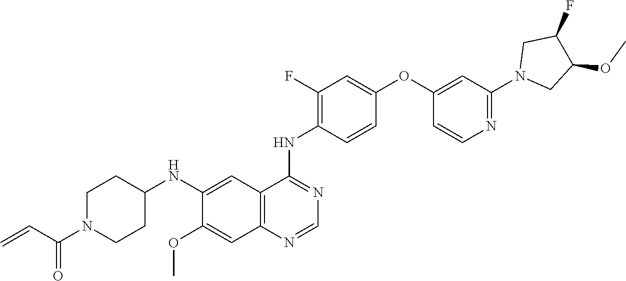 [SMILES: C=CC(=O)N1CCC(Nc2cc3c(Nc4ccc(Oc5ccnc(N6C[C@@H](F)[C@@H](OC)C6)c5)cc4F)ncnc3cc2OC)CC1] | 1-(4-((4-((2-fluoro- 4-((2-((3R,4S)-3- fluoro-4- methoxypyrrolidin-1- yl)pyridin-4-yl)oxy) phenyl)amino)-7- methoxyquinazolin-6- yl)amino)piperidin- 1-yl)prop-2-en-1- one |
| 264 | 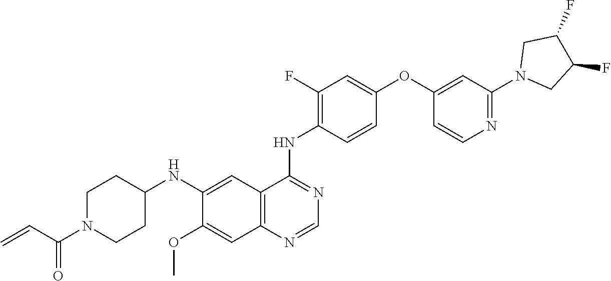 [SMILES: C=CC(=O)N1CCC(Nc2cc3c(Nc4ccc(Oc5ccnc(N6C[C@H](F)[C@@H](F)C6)c5)cc4F)ncnc3cc2OC)CC1] | 1-(4-((4-((4-((2-((3S,4S)-3,4- difluoropyrrolidin- 1-yl)pyridin-4- yl)oxy)-2-fluorophenyl) amino)-7- methoxyquinazolin-6- yl)amino)piperidin- 1-yl)prop-2-en-1- one |
| 265 | 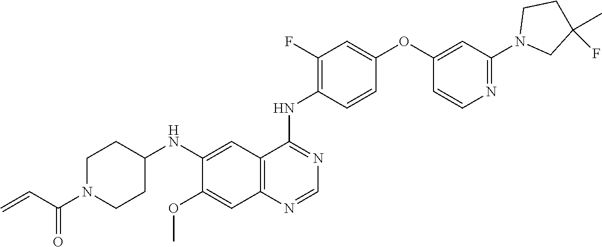 [SMILES: C=CC(=O)N1CCC(Nc2cc3c(Nc4ccc(Oc5ccnc(N6CCC(C)(F)C6)c5)cc4F)ncnc3cc2OC)CC1] | 1-(4-((4-((2-fluoro- 4-((2-(3-fluoro-3- methylpyrrolidin- 1-yl)pyridin-4- yl)oxy)phenyl)amino)-7- methoxyquinazolin-6- yl)amino)piperidin- 1-yl)prop-2-en-1- one |
| 266 | 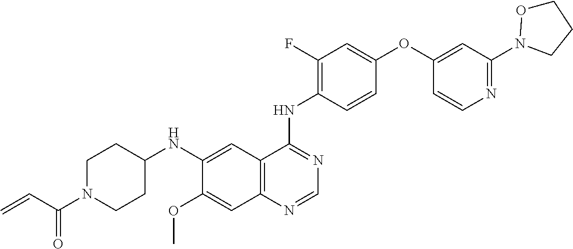 [SMILES: C=CC(=O)N1CCC(Nc2cc3c(Nc4ccc(Oc5ccnc(N6CCCO6)c5)cc4F)ncnc3cc2OC)CC1] | 1-(4-((4-((2-fluoro-4-((2- (isoxazolidin-2-yl)pyridin-4- yl)oxy)phenyl)amino)-7- methoxyquinazolin-6- yl)amino)piperidin- 1-yl)prop-2-en-1- one |
| 267 | 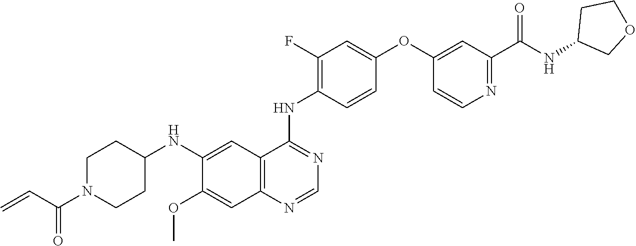 [SMILES: C=CC(=O)N1CCC(Nc2cc3c(Nc4ccc(Oc5ccnc(C(=O)N[C@@H]6CCOC6)c5)cc4F)ncnc3cc2OC)CC1] | (R)-4-(4-((6-((1- acryloylpiperidin-4- yl)amino)-7- methoxyquinazolin-4- yl)amino)-3- fluorophenoxy)-N- (tetrahydrofuran-3- yl)picolinamide |
| 268 | 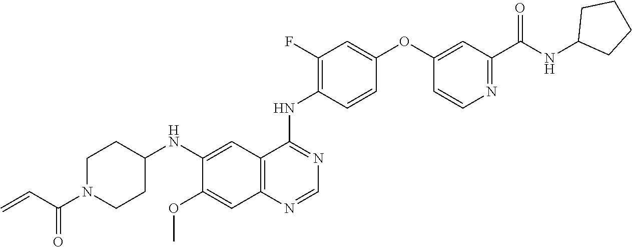 [SMILES: C=CC(=O)N1CCC(Nc2cc3c(Nc4ccc(Oc5ccnc(C(=O)NC6CCCC6)c5)cc4F)ncnc3cc2OC)CC1] | 4-(4-((6-((1- acryloylpiperidin-4- yl)amino)-7- methoxyquinazolin-4- yl)amino)-3- fluorophenoxy)-N- cyclopentylpicolinamide |
| 269 | 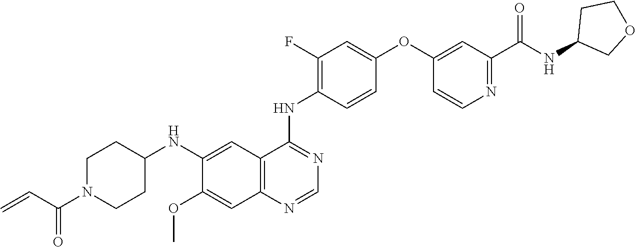 [SMILES: C=CC(=O)N1CCC(Nc2cc3c(Nc4ccc(Oc5ccnc(C(=O)N[C@H]6CCOC6)c5)cc4F)ncnc3cc2OC)CC1] | (S)-4-(4-((6-((1- acryloylpiperidin-4- yl)amino)-7- methoxyquinazolin-4- yl)amino)-3- fluorophenoxy)-N- (tetrahydrofuran-3- yl)picolinamide |
| 270 | 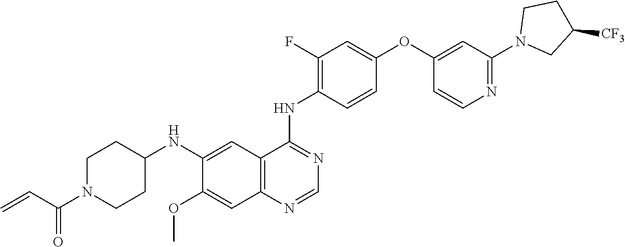 [SMILES: C=CC(=O)N1CCC(Nc2cc3c(Nc4ccc(Oc5ccnc(N6CC[C@@H](C(F)(F)F)C6)c5)cc4F)ncnc3cc2OC)CC1] | (R)-1-(4-((4-((2- fluoro-4-((2-(3- (trifluoromethyl) pyrrolidin-1- yl)pyridin-4-yl)oxy) phenyl)amino)-7- methoxyquinazolin-6- yl)amino)piperidin- 1-yl)prop-2-en-1- one |
| 271 | 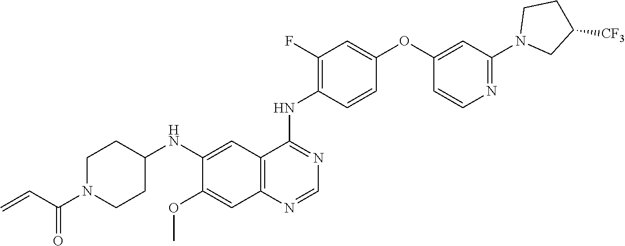 [SMILES: C=CC(=O)N1CCC(Nc2cc3c(Nc4ccc(Oc5ccnc(N6CC[C@H](C(F)(F)F)C6)c5)cc4F)ncnc3cc2OC)CC1] | (S)-1-(4-((4-((2- fluoro-4-((2-(3- (trifluoromethyl)pyrrolidin-1- yl)pyridin-4-yl)oxy) phenyl)amino)-7- methoxyquinazolin-6- yl)amino)piperidin- 1-yl)prop-2-en-1- one |
| 272 | 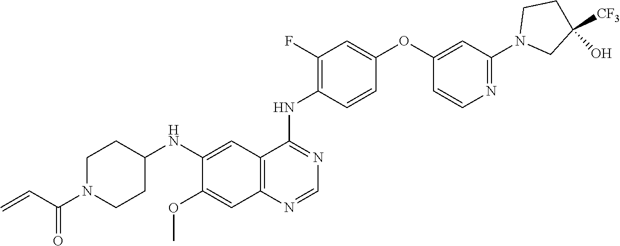 [SMILES: C=CC(=O)N1CCC(Nc2cc3c(Nc4ccc(Oc5ccnc(N6CC[C@@](O)(C(F)(F)F)C6)c5)cc4F)ncnc3cc2OC)CC1] | (S)-1-(4-((4-((2- fluoro-4-((2-(3- hydroxy-3-(trifluoromethyl) pyrrolidin- 1-yl)pyridin-4-yl)oxy) phenyl)amino)- 7-methoxyquinazolin-6- yl)amino)piperidin- 1-yl)prop-2-en-1- one |
| 273 | 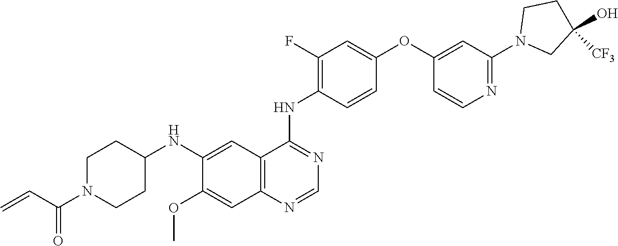 [SMILES: C=CC(=O)N1CCC(Nc2cc3c(Nc4ccc(Oc5ccnc(N6CC[C@](O)(C(F)(F)F)C6)c5)cc4F)ncnc3cc2OC)CC1] | (R)-1-(4-((4-((2- fluoro-4-((2-(3- hydroxy-3-(trifluoromethyl) pyrrolidin- 1-yl)pyridin-4-yl)oxy) phenyl)amino)- 7-methoxyquinazolin-6- yl)amino)piperidin- 1-yl)prop-2-en-1- one |
| 274 | 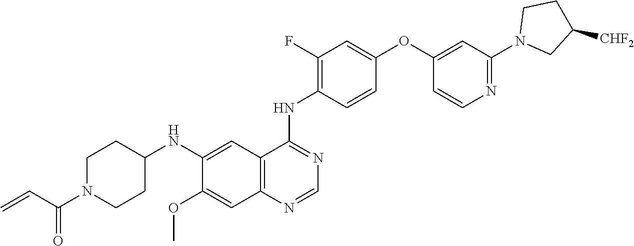 [SMILES: C=CC(=O)N1CCC(Nc2cc3c(Nc4ccc(Oc5ccnc(N6CC[C@@H](C(F)F)C6)c5)cc4F)ncnc3cc2OC)CC1] | (R)-1-(4-((4-((4-((2-(3- (difluoromethyl)pyrrolidin-1- yl)pyridin-4-yl)oxy)-2- fluorophenyl)amino)-7- methoxyquinazolin-6- yl)amino)piperidin- 1-yl)prop-2-en-1- one |
| 275 | 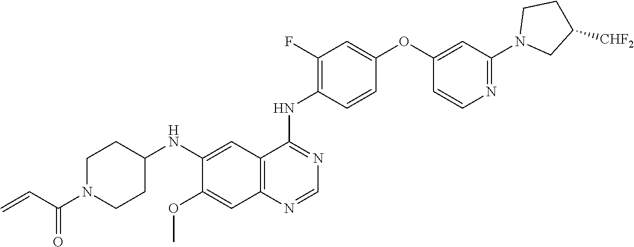 [SMILES: C=CC(=O)N1CCC(Nc2cc3c(Nc4ccc(Oc5ccnc(N6CC[C@H](C(F)F)C6)c5)cc4F)ncnc3cc2OC)CC1] | (S)-1-(4-((4-((4-((2-(3- (difluoromethyl)pyrrolidin-1- yl)pyridin-4-yl)oxy)-2- fluorophenyl)amino)-7- methoxyquinazolin-6- yl)amino)piperidin- 1-yl)prop-2-en-1- one |
| 276 | 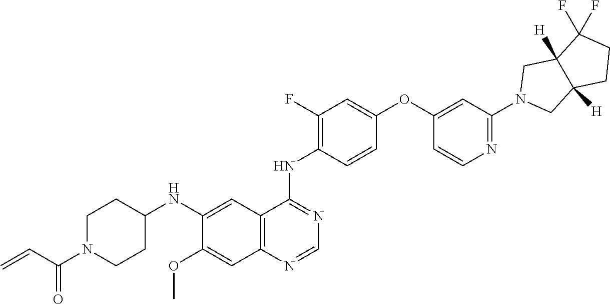 [SMILES: C=CC(=O)N1CCC(Nc2cc3c(Nc4ccc(Oc5ccnc(N6C[C@H]7CCC(F)(F)[C@H]7C6)c5)cc4F)ncnc3cc2OC)CC1] | 1-(4-((4-((4-((2- ((3aR,6aS)-4,4- difluorohexahydro- cyclopenta[c] pyrrol-2(1H)-yl) pyridin-4-yl)oxy)-2- fluorophenyl)amino)-7- methoxyquinazolin-6- yl)amino)piperidin- 1-yl)prop-2-en-1- one |
| 277 | 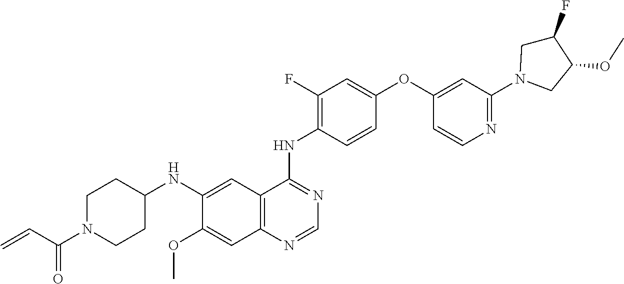 [SMILES: C=CC(=O)N1CCC(Nc2cc3c(Nc4ccc(Oc5ccnc(N6C[C@@H](F)[C@H](OC)C6)c5)cc4F)ncnc3cc2OC)CC1] | 1-(4-((4-((2-fluoro- 4-((2-((3R,4R)-3- fluoro-4-methoxypyrrolidin-1- yl)pyridin-4-yl)oxy) phenyl)amino)-7- methoxyquinazolin-6- yl)amino)piperidin- 1-yl)prop-2-en-1- one |
| 278 | 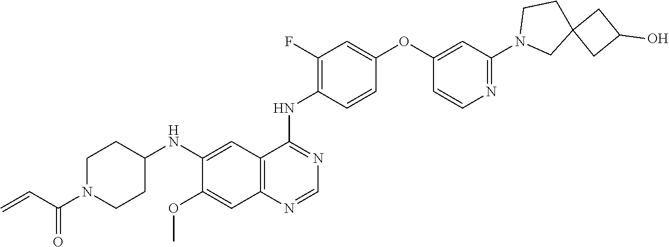 [SMILES: C=CC(=O)N1CCC(Nc2cc3c(Nc4ccc(Oc5ccnc(N6CCC7(CC(O)C7)C6)c5)cc4F)ncnc3cc2OC)CC1] | 1-(4-((4-((2-fluoro- 4-((2-(2-hydroxy- 6-azaspiro[3.4]octan- 6-yl)pyridin-4- yl)oxy)phenyl)amino)-7- methoxyquinazolin-6- yl)amino)piperidin- 1-yl)prop-2-en-1- one |
| 279 | 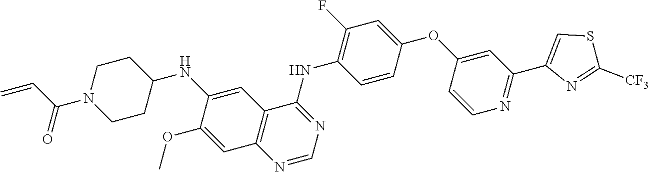 [SMILES: C=CC(=O)N1CCC(Nc2cc3c(Nc4ccc(Oc5ccnc(-c6csc(C(F)(F)F)n6)c5)cc4F)ncnc3cc2OC)CC1] | 1-(4-((4-((2-fluoro-4-((2-(2- (trifluoromethyl) thiazol-4-yl)pyridin- 4-yl)oxy)phenyl)amino)-7- methoxyquinazolin-6- yl)amino)piperidin- 1-yl)prop-2-en-1- one |
| 280 | 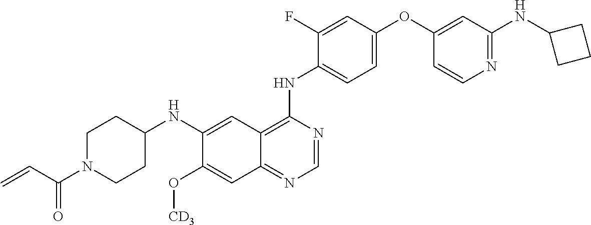 [SMILES: [2H]C([2H])([2H])Oc1cc2ncnc(Nc3ccc(Oc4ccnc(NC5CCC5)c4)cc3F)c2cc1NC1CCN(C(=O)C=C)CC1] | 1-(4-((4-((4-((2- (cyclobutylamino) pyridin-4-yl)oxy)-2- fluorophenyl)amino)- 7-(methoxy- d3)quinazolin-6-yl) amino)piperidin- 1-yl)prop-2-en-1-one |
| 281 | 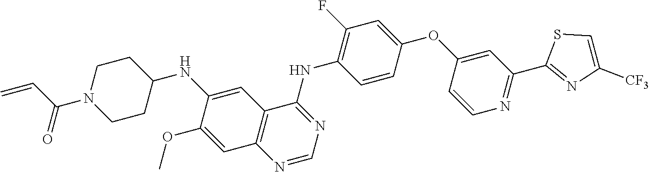 [SMILES: C=CC(=O)N1CCC(Nc2cc3c(Nc4ccc(Oc5ccnc(-c6nc(C(F)(F)F)cs6)c5)cc4F)ncnc3cc2OC)CC1] | 1-(4-((4-((2-fluoro-4-((2-(4- (trifluoromethyl) thiazol-2-yl)pyridin- 4-yl)oxy)phenyl)amino)-7- methoxyquinazolin-6- yl)amino)piperidin- 1-yl)prop-2-en-1- one |
| 282 | 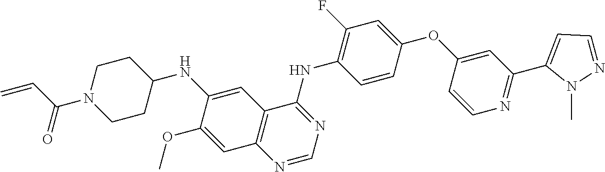 [SMILES: C=CC(=O)N1CCC(Nc2cc3c(Nc4ccc(Oc5ccnc(-c6ccnn6C)c5)cc4F)ncnc3cc2OC)CC1] | 1-(4-((4-((2-fluoro- 4-((2-(1-methyl- 1H-pyrazol-5-yl)pyridin-4- yl)oxy)phenyl)amino)-7- methoxyquinazolin-6- yl)amino)piperidin- 1-yl)prop-2-en-1- one |
| 283 | 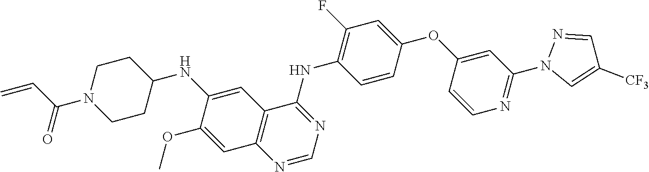 [SMILES: C=CC(=O)N1CCC(Nc2cc3c(Nc4ccc(Oc5ccnc(-n6cc(C(F)(F)F)cn6)c5)cc4F)ncnc3cc2OC)CC1] | 1-(4-((4-((2-fluoro-4-((2-(4- (trifluoromethyl)- 1H-pyrazol-1- yl)pyridin-4-yl)oxy) phenyl)amino)-7- methoxyquinazolin-6- yl)amino)piperidin- 1-yl)prop-2-en-1- one |
| 284 | 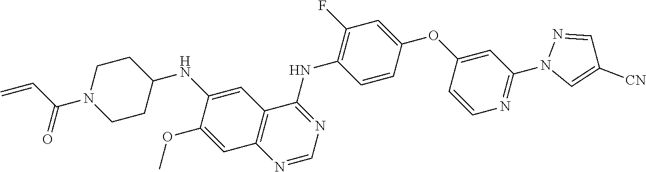 [SMILES: C=CC(=O)N1CCC(Nc2cc3c(Nc4ccc(Oc5ccnc(-n6cc(C#N)cn6)c5)cc4F)ncnc3cc2OC)CC1] | 1-(4-(4-((6-((1- acryloylpiperidin-4- yl)amino)-7- methoxyquinazolin-4- yl)amino)-3-fluorophenoxy) pyridin-2- yl)-1H-pyrazole- 4-carbonitrile |
| 285 | 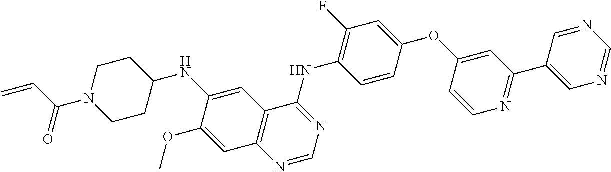 [SMILES: C=CC(=O)N1CCC(Nc2cc3c(Nc4ccc(Oc5ccnc(-c6cncnc6)c5)cc4F)ncnc3cc2OC)CC1] | 1-(4-((4-((2-fluoro- 4-((2-(pyrimidin-5- yl)pyridin-4-yl)oxy) phenyl)amino)-7- methoxyquinazolin-6- yl)amino)piperidin- 1-yl)prop-2-en-1- one |
| 286 | 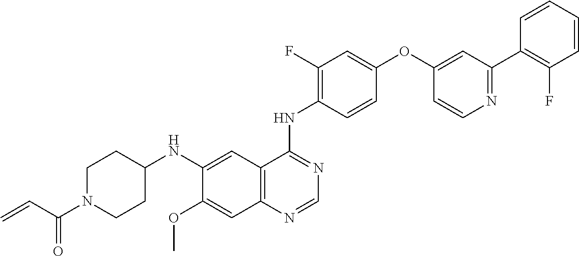 [SMILES: C=CC(=O)N1CCC(Nc2cc3c(Nc4ccc(Oc5ccnc(-c6ccccc6F)c5)cc4F)ncnc3cc2OC)CC1] | 1-(4-((4-((2-fluoro-4-((2-(2- fluorophenyl)pyridin-4- yl)oxy)phenyl)amino)-7- methoxyquinazolin-6- yl)amino)piperidin- 1-yl)prop-2-en-1- one |
| 287 | 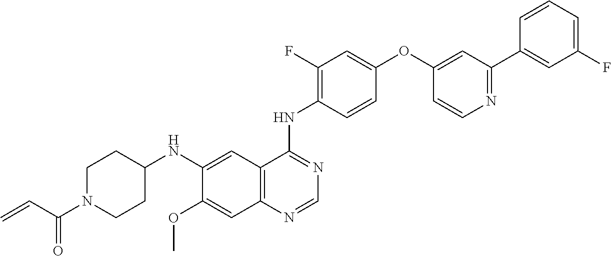 [SMILES: C=CC(=O)N1CCC(Nc2cc3c(Nc4ccc(Oc5ccnc(-c6cccc(F)c6)c5)cc4F)ncnc3cc2OC)CC1] | 1-(4-((4-((2-fluoro-4-((2-(3- fluorophenyl)pyridin-4- yl)oxy)phenyl)amino)-7- methoxyquinazolin-6- yl)amino)piperidin- 1-yl)prop-2-en-1- one |
| 288 | 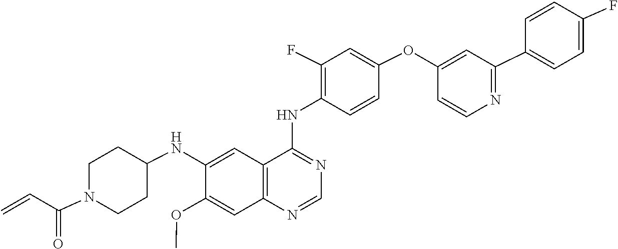 [SMILES: C=CC(=O)N1CCC(Nc2cc3c(Nc4ccc(Oc5ccnc(-c6ccc(F)cc6)c5)cc4F)ncnc3cc2OC)CC1] | 1-(4-((4-((2-fluoro-4-((2-(4- fluorophenyl)pyridin-4- yl)oxy)phenyl)amino)-7- methoxyquinazolin-6- yl)amino)piperidin- 1-yl)prop-2-en-1- one |
| 289 | 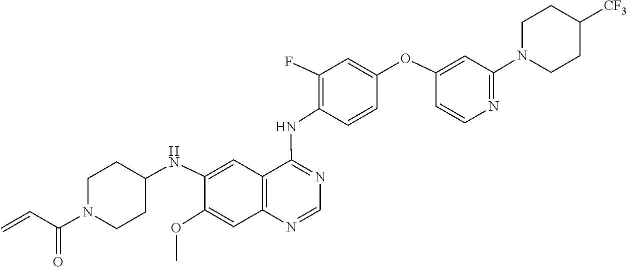 [SMILES: C=CC(=O)N1CCC(Nc2cc3c(Nc4ccc(Oc5ccnc(N6CCC(C(F)(F)F)CC6)c5)cc4F)ncnc3cc2OC)CC1] | 1-(4-((4-((2-fluoro-4-((2-(4- (trifluoromethyl)piperidin-1- yl)pyridin-4-yl)oxy) phenyl)amino)-7- methoxyquinazolin-6- yl)amino)piperidin- 1-yl)prop-2-en-1- one |
| 290 | 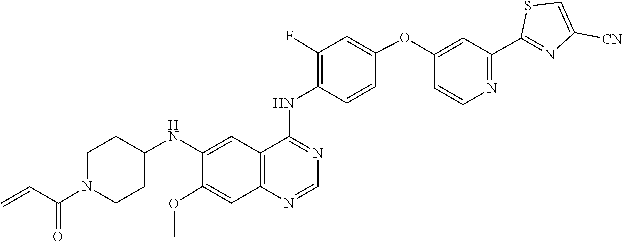 [SMILES: C=CC(=O)N1CCC(Nc2cc3c(Nc4ccc(Oc5ccnc(-c6nc(C#N)cs6)c5)cc4F)ncnc3cc2OC)CC1] | 2-(4-(4-((6-((1- acryloylpiperidin-4- yl)amino)-7- methoxyquinazolin-4- yl)amino)-3-fluorophenoxy) pyridin-2- yl)thiazole-4-carbonitrile |
| 291 | 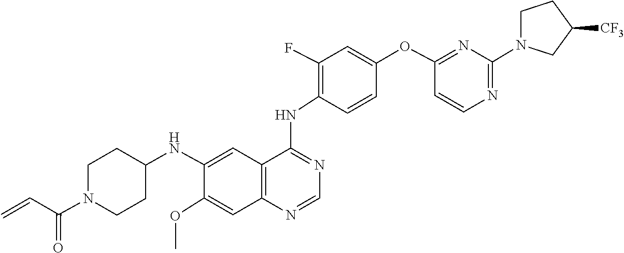 [SMILES: C=CC(=O)N1CCC(Nc2cc3c(Nc4ccc(Oc5ccnc(N6CC[C@@H](C(F)(F)F)C6)n5)cc4F)ncnc3cc2OC)CC1] | (R)-1-(4-((4-((2- fluoro-4-((2-(3- (trifluoromethyl)pyrrolidin-1- yl)pyrimidin-4-yl) oxy)phenyl)amino)- 7-methoxyquinazolin-6- yl)amino)piperidin- 1-yl)prop-2-en-1- one |
| 292 | 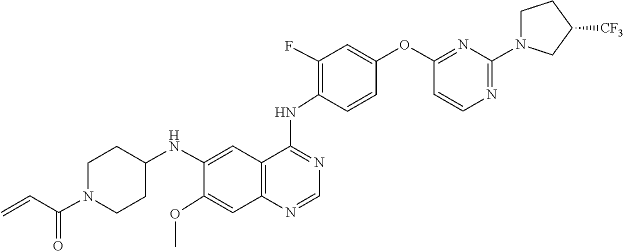 [SMILES: C=CC(=O)N1CCC(Nc2cc3c(Nc4ccc(Oc5ccnc(N6CC[C@H](C(F)(F)F)C6)n5)cc4F)ncnc3cc2OC)CC1] | (S)-1-(4-((4-((2- fluoro-4-((2-(3- (trifluoromethyl)pyrrolidin-1- yl)pyrimidin-4-yl) oxy)phenyl)amino)- 7-methoxyquinazolin-6- yl)amino)piperidin- 1-yl)prop-2-en-1- one |
| 293 | 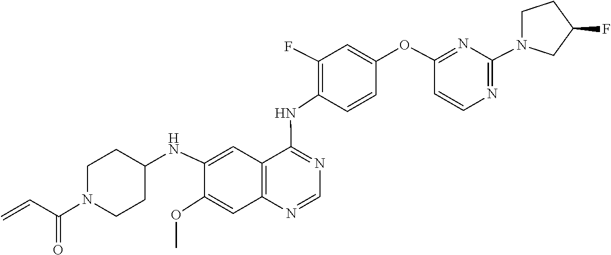 [SMILES: C=CC(=O)N1CCC(Nc2cc3c(Nc4ccc(Oc5ccnc(N6CC[C@@H](F)C6)n5)cc4F)ncnc3cc2OC)CC1] | (R)-1-(4-((4-((2- fluoro-4-((2-(3- fluoropyrrolidin- 1-yl)pyrimidin-4- yl)oxy)phenyl)amino)-7- methoxyquinazolin-6- yl)amino)piperidin- 1-yl)prop-2-en-1- one |
| 294 | 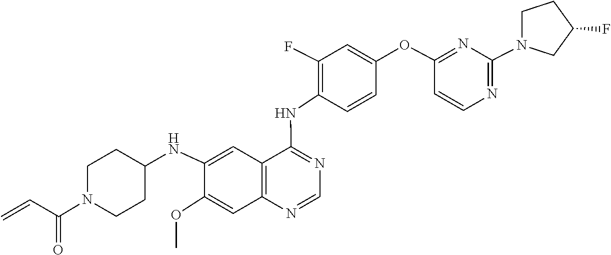 [SMILES: C=CC(=O)N1CCC(Nc2cc3c(Nc4ccc(Oc5ccnc(N6CC[C@H](F)C6)n5)cc4F)ncnc3cc2OC)CC1] | (S)-1-(4-((4-((2- fluoro-4-((2-(3- fluoropyrrolidin- 1-yl)pyrimidin-4- yl)oxy)phenyl)amino)-7- methoxyquinazolin-6- yl)amino)piperidin- 1-yl)prop-2-en-1- one |
| 295 | 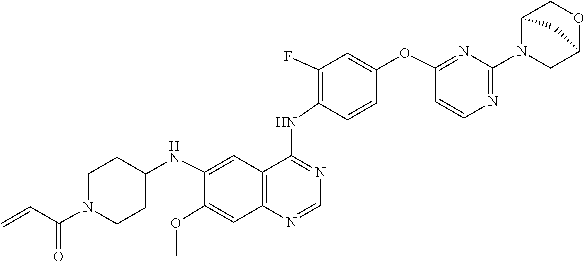 [SMILES: C=CC(=O)N1CCC(Nc2cc3c(Nc4ccc(Oc5ccnc(N6C[C@H]7C[C@@H]6CO7)n5)cc4F)ncnc3cc2OC)CC1] | 1-(4-((4-((4-((2- ((1R,4R)-2-oxa-5- azabicyclo[2.2.1]heptan-5- yl)pyrimidin-4-yl)oxy)-2- fluorophenyl)amino)-7- methoxyquinazolin-6- yl)amino)piperidin- 1-yl)prop-2-en-1- one |
| 296 | 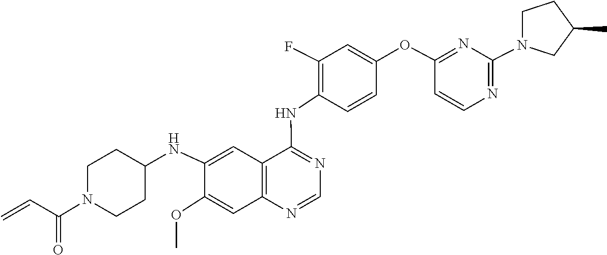 [SMILES: C=CC(=O)N1CCC(Nc2cc3c(Nc4ccc(Oc5ccnc(N6CC[C@@H](C)C6)n5)cc4F)ncnc3cc2OC)CC1] | (R)-1-(4-((4-((2- fluoro-4-((2-(3- methylpyrrolidin- 1-yl)pyrimidin-4- yl)oxy)phenyl)amino)-7- methoxyquinazolin-6- yl)amino)piperidin- 1-yl)prop-2-en-1- one |
| 297 | 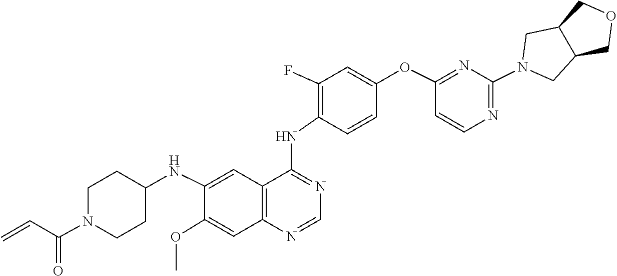 [SMILES: C=CC(=O)N1CCC(Nc2cc3c(Nc4ccc(Oc5ccnc(N6C[C@H]7COC[C@H]7C6)n5)cc4F)ncnc3cc2OC)CC1] | 1-(4-((4-((2-fluoro- 4-((2-((3aR,6aS)- tetrahydro-1H- furo[3,4-c]pyrrol- 5(3H)-yl)pyrimidin-4- yl)oxy)phenyl)amino)-7- methoxyquinazolin-6- yl)amino)piperidin- 1-yl)prop-2-en-1- one |
| 298 | 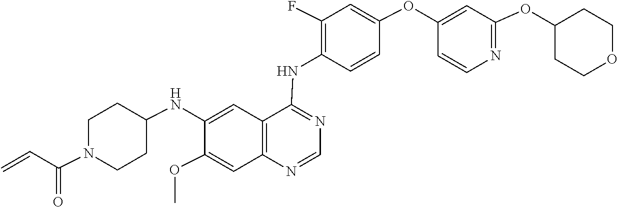 [SMILES: C=CC(=O)N1CCC(Nc2cc3c(Nc4ccc(Oc5ccnc(OC6CCOCC6)c5)cc4F)ncnc3cc2OC)CC1] | 1-(4-((4-((2-fluoro- 4-((2-((tetrahydro- 2H-pyran-4-yl)oxy)pyridin-4- yl)oxy)phenyl)amino)-7- methoxyquinazolin-6- yl)amino)piperidin- 1-yl)prop-2-en-1- one |
| 299 | 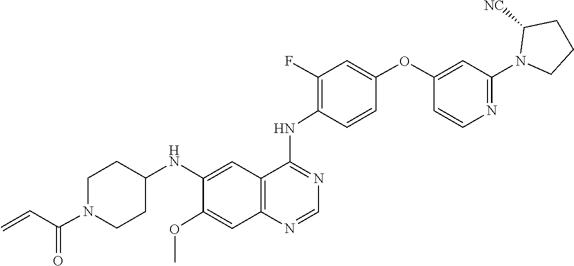 [SMILES: C=CC(=O)N1CCC(Nc2cc3c(Nc4ccc(Oc5ccnc(N6CCC[C@H]6C#N)c5)cc4F)ncnc3cc2OC)CC1] | (S)-1-(4-(4-((6-((1- acryloylpiperidin- 4-yl)amino)-7- methoxyquinazolin-4- yl)amino)-3-fluorophenoxy) pyridin-2- yl)pyrrolidine-2-carbonitrile |
| 300 | 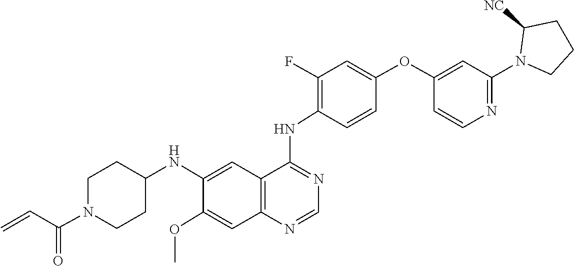 [SMILES: C=CC(=O)N1CCC(Nc2cc3c(Nc4ccc(Oc5ccnc(N6CCC[C@@H]6C#N)c5)cc4F)ncnc3cc2OC)CC1] | (R)-1-(4-(4-((6-((1- acryloylpiperidin- 4-yl)amino)-7- methoxyquinazolin-4- yl)amino)-3-fluorophenoxy) pyridin-2- yl)pyrrolidine-2-carbonitrile |
| 301 | 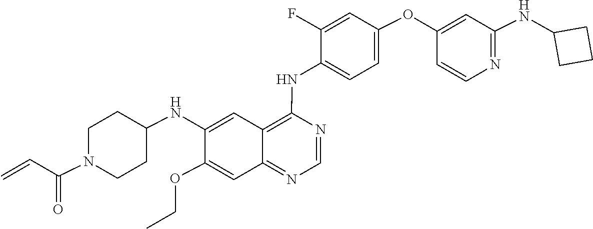 [SMILES: C=CC(=O)N1CCC(Nc2cc3c(Nc4ccc(Oc5ccnc(NC6CCC6)c5)cc4F)ncnc3cc2OCC)CC1] | 1-(4-((4-((4-((2- (cyclobutylamino) pyridin-4-yl)oxy)-2- fluorophenyl)amino)-7- ethoxyquinazolin-6- yl)amino)piperidin- 1-yl)prop-2-en-1- one |
| 302 | 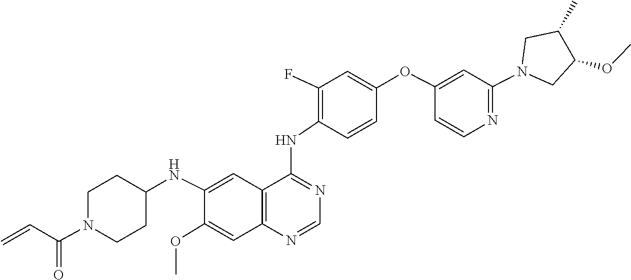 [SMILES: C=CC(=O)N1CCC(Nc2cc3c(Nc4ccc(Oc5ccnc(N6C[C@H](C)[C@H](OC)C6)c5)cc4F)ncnc3cc2OC)CC1] | 1-(4-((4-((2-fluoro- 4-((2-((3S,4S)-3- methoxy-4- methylpyrrolidin-1- yl)pyridin-4-yl)oxy) phenyl)amino)-7- methoxyquinazolin-6- yl)amino)piperidin- 1-yl)prop-2-en-1- one |
| 303 | 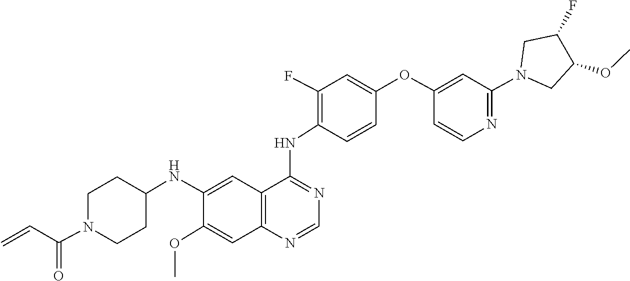 [SMILES: C=CC(=O)N1CCC(Nc2cc3c(Nc4ccc(Oc5ccnc(N6C[C@H](F)[C@H](OC)C6)c5)cc4F)ncnc3cc2OC)CC1] | 1-(4-((4-((2-fluoro- 4-((2-((3S,4R)-3- fluoro-4- methoxypyrrolidin-1- yl)pyridin-4-yl)oxy) phenyl)amino)-7- methoxyquinazolin-6- yl)amino)piperidin- 1-yl)prop-2-en-1- one |
| 304 | 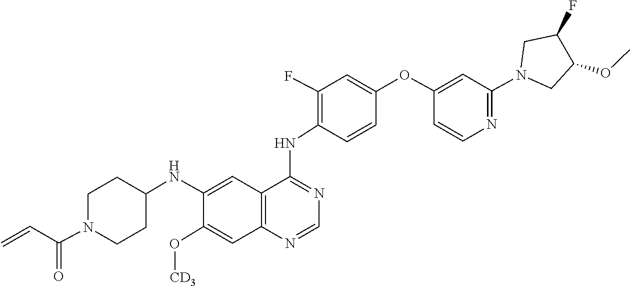 [SMILES: [2H]C([2H])([2H])Oc1cc2ncnc(Nc3ccc(Oc4ccnc(N5C[C@@H](F)[C@H](OC)C5)c4)cc3F)c2cc1NC1CCN(C(=O)C=C)CC1] | 1-(4-((4-((2-fluoro- 4-((2-((3R,4R)-3- fluoro-4- methoxypyrrolidin-1- yl)pyridin-4-yl)oxy) phenyl)amino)-7- (methoxy-d3)quinazolin-6- yl)amino)piperidin- 1-yl)prop-2-en-1- one |
| 305 | 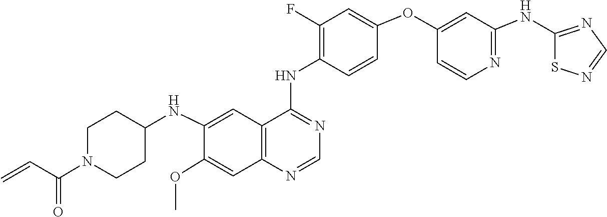 [SMILES: C=CC(=O)N1CCC(Nc2cc3c(Nc4ccc(Oc5ccnc(Nc6ncns6)c5)cc4F)ncnc3cc2OC)CC1] | 1-(4-((4-((4-((2- ((1,2,4-thiadiazol-5- yl)amino)pyridin- 4-yl)oxy)-2- fluorophenyl)amino)-7- methoxyquinazolin-6- yl)amino)piperidin- 1-yl)prop-2-en-1- one |
| 306 | 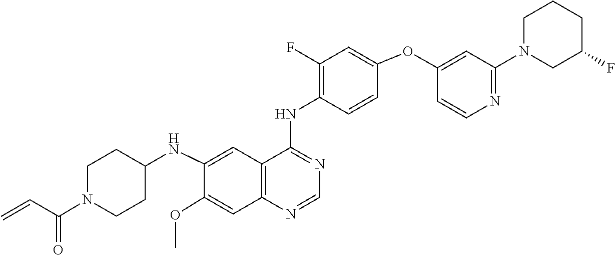 [SMILES: C=CC(=O)N1CCC(Nc2cc3c(Nc4ccc(Oc5ccnc(N6CCC[C@H](F)C6)c5)cc4F)ncnc3cc2OC)CC1] | (S)-1-(4-((4-((2- fluoro-4-((2-(3- fluoropiperidin-1- yl)pyridin-4- yl)oxy)phenyl)amino)-7- methoxyquinazolin-6- yl)amino)piperidin- 1-yl)prop-2-en-1- one |
| 307 | 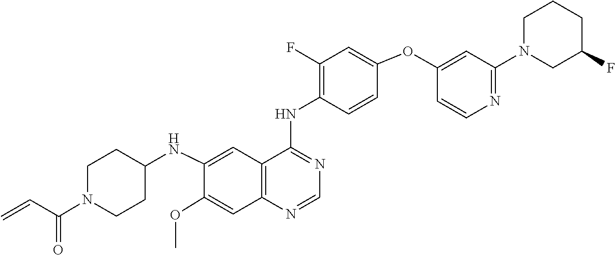 [SMILES: C=CC(=O)N1CCC(Nc2cc3c(Nc4ccc(Oc5ccnc(N6CCC[C@@H](F)C6)c5)cc4F)ncnc3cc2OC)CC1] | (R)-1-(4-((4-((2- fluoro-4-((2-(3- fluoropiperidin- 1-yl)pyridin-4- yl)oxy)phenyl)amino)-7- methoxyquinazolin-6- yl)amino)piperidin- 1-yl)prop-2-en-1- one |
| 308 | 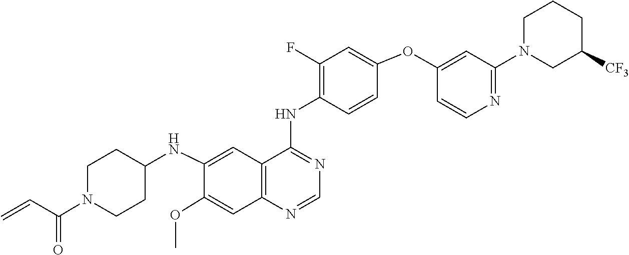 [SMILES: C=CC(=O)N1CCC(Nc2cc3c(Nc4ccc(Oc5ccnc(N6CCC[C@@H](C(F)(F)F)C6)c5)cc4F)ncnc3cc2OC)CC1] | (R)-1-(4-((4-((2- fluoro-4-((2-(3- (trifluoromethyl)piperidin-1- yl)pyridin-4-yl)oxy) phenyl)amino)-7- methoxyquinazolin-6- yl)amino)piperidin- 1-yl)prop-2-en-1- one |
| 309 | 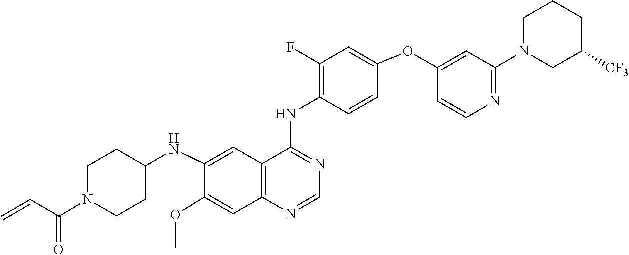 [SMILES: C=CC(=O)N1CCC(Nc2cc3c(Nc4ccc(Oc5ccnc(N6CCC[C@H](C(F)(F)F)C6)c5)cc4F)ncnc3cc2OC)CC1] | (S)-1-(4-((4-((2- fluoro-4-((2-(3- (trifluoromethyl)piperidin-1- yl)pyridin-4-yl)oxy) phenyl)amino)-7- methoxyquinazolin-6- yl)amino)piperidin- 1-yl)prop-2-en-1- one |
| 310 | 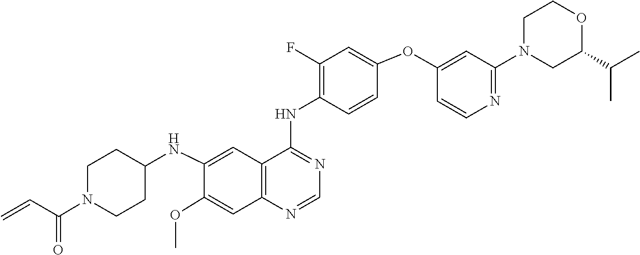 [SMILES: C=CC(=O)N1CCC(Nc2cc3c(Nc4ccc(Oc5ccnc(N6CCO[C@H](C(C)C)C6)c5)cc4F)ncnc3cc2OC)CC1] | (R)-1-(4-((4-((2- fluoro-4-((2-(2- isopropylmorpholino) pyridin-4- yl)oxy)phenyl)amino)-7- methoxyquinazolin-6- yl)amino)piperidin- 1-yl)prop-2-en-1- one |
| 311 | 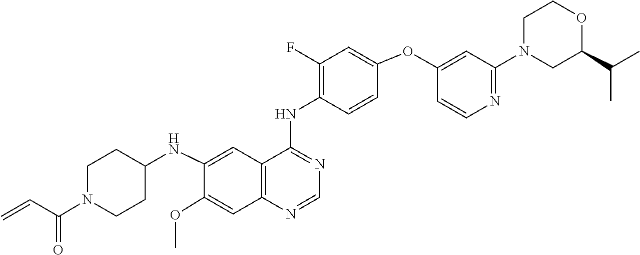 [SMILES: C=CC(=O)N1CCC(Nc2cc3c(Nc4ccc(Oc5ccnc(N6CCO[C@@H](C(C)C)C6)c5)cc4F)ncnc3cc2OC)CC1] | (S)-1-(4-((4-((2- fluoro-4-((2-(2- isopropylmorpholino) pyridin-4- yl)oxy)phenyl)amino)-7- methoxyquinazolin-6- yl)amino)piperidin- 1-yl)prop-2-en-1- one |
| 312 | 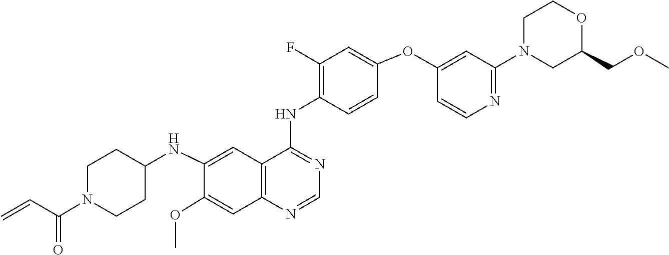 [SMILES: C=CC(=O)N1CCC(Nc2cc3c(Nc4ccc(Oc5ccnc(N6CCO[C@@H](COC)C6)c5)cc4F)ncnc3cc2OC)CC1] | (R)-1-(4-((4-((2- fluoro-4-((2-(2- (methoxymethyl) morpholino)pyridin- 4-yl)oxy)phenyl)amino)-7- methoxyquinazolin-6- yl)amino)piperidin- 1-yl)prop-2-en-1- one |
| 313 | 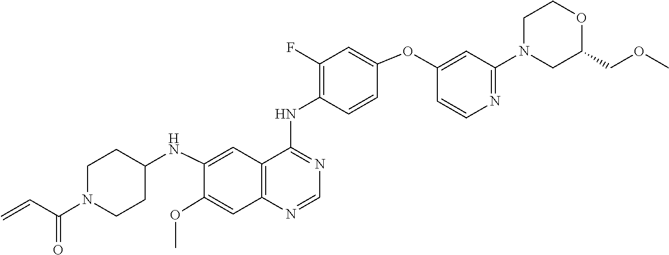 [SMILES: C=CC(=O)N1CCC(Nc2cc3c(Nc4ccc(Oc5ccnc(N6CCO[C@H](COC)C6)c5)cc4F)ncnc3cc2OC)CC1] | (S)-1-(4-((4-((2- fluoro-4-((2-(2- (methoxymethyl) morpholino)pyridin- 4-yl)oxy)phenyl)amino)-7- methoxyquinazolin-6- yl)amino)piperidin- 1-yl)prop-2-en-1- one |
| 314 | 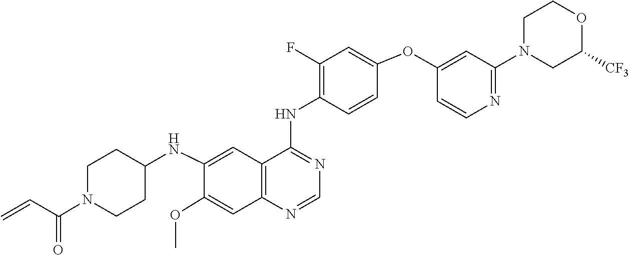 [SMILES: C=CC(=O)N1CCC(Nc2cc3c(Nc4ccc(Oc5ccnc(N6CCO[C@H](C(F)(F)F)C6)c5)cc4F)ncnc3cc2OC)CC1] | (S)-1-(4-((4-((2- fluoro-4-((2-(2- (trifluoromethyl) morpholino)pyridin- 4-yl)oxy)phenyl)amino)-7- methoxyquinazolin-6- yl)amino)piperidin- 1-yl)prop-2-en-1- one |
| 315 | 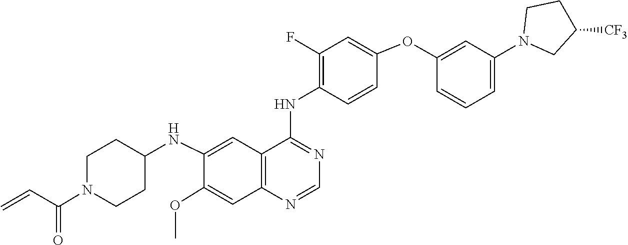 [SMILES: C=CC(=O)N1CCC(Nc2cc3c(Nc4ccc(Oc5cccc(N6CC[C@H](C(F)(F)F)C6)c5)cc4F)ncnc3cc2OC)CC1] | (S)-1-(4-((4-((2- fluoro-4-(3-(3- (trifluoromethyl) pyrrolidin-1- yl)phenoxy) phenyl)amino)-7- methoxyquinazolin-6- yl)amino)piperidin- 1-yl)prop-2-en-1- one |
| 316 | 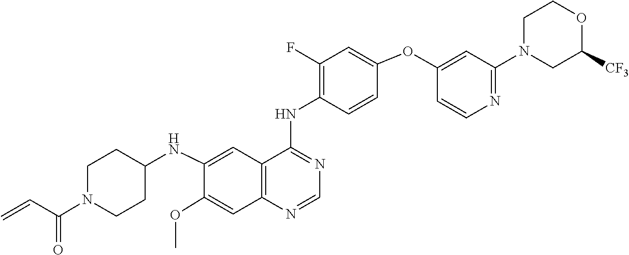 [SMILES: C=CC(=O)N1CCC(Nc2cc3c(Nc4ccc(Oc5ccnc(N6CCO[C@@H](C(F)(F)F)C6)c5)cc4F)ncnc3cc2OC)CC1] | (R)-1-(4-((4-((2- fluoro-4-((2-(2- (trifluoromethyl) morpholino)pyridin- 4-yl)oxy)phenyl)amino)-7- methoxyquinazolin-6- yl)amino)piperidin- 1-yl)prop-2-en-1- one |
| 317 | 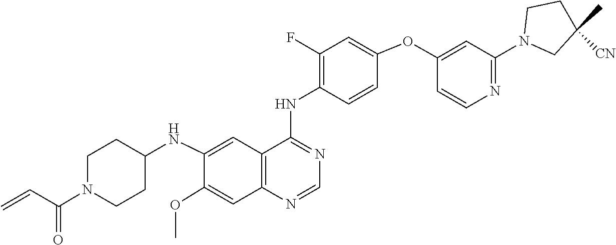 [SMILES: C=CC(=O)N1CCC(Nc2cc3c(Nc4ccc(Oc5ccnc(N6CC[C@](C)(C#N)C6)c5)cc4F)ncnc3cc2OC)CC1] | (S)-1-(4-(4-((6-((1- acryloylpiperidin- 4-yl)amino)-7- methoxyquinazolin-4- yl)amino)-3-fluorophenoxy) pyridin-2- yl)-3-methylpyrrolidine- 3-carbonitrile |
| 318 | 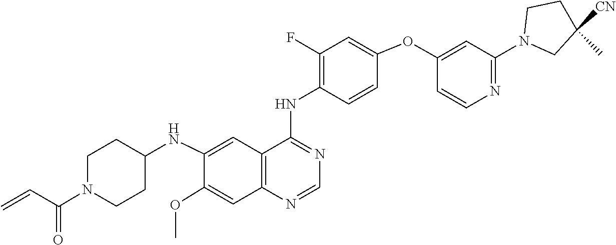 [SMILES: C=CC(=O)N1CCC(Nc2cc3c(Nc4ccc(Oc5ccnc(N6CC[C@@](C)(C#N)C6)c5)cc4F)ncnc3cc2OC)CC1] | (R)-1-(4-(4-((6-((1- acryloylpiperidin- 4-yl)amino)-7- methoxyquinazolin-4- yl)amino)-3-fluorophenoxy )pyridin-2- yl)-3-methylpyrrolidine- 3-carbonitrile |
| 319 | 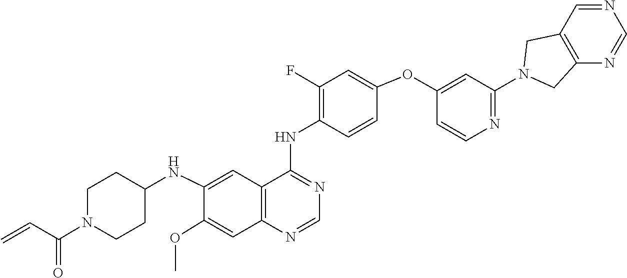 [SMILES: C=CC(=O)N1CCC(Nc2cc3c(Nc4ccc(Oc5ccnc(N6Cc7cncnc7C6)c5)cc4F)ncnc3cc2OC)CC1] | 1-(4-((4-((4-((2- (5,7-dihydro-6H- pyrrolo[3,4-d]pyrimidin- 6-yl)pyridin- 4-yl)oxy)-2- fluorophenyl)amino)-7- methoxyquinazolin-6- yl)amino)piperidin- 1-yl)prop-2-en-1- one |
| 320 | 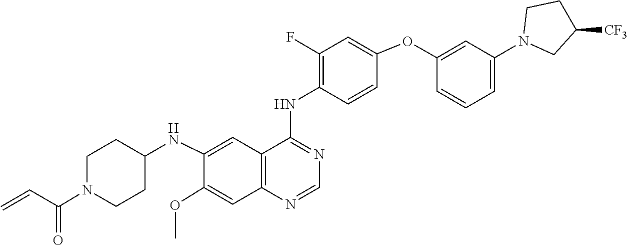 [SMILES: C=CC(=O)N1CCC(Nc2cc3c(Nc4ccc(Oc5cccc(N6CC[C@@H](C(F)(F)F)C6)c5)cc4F)ncnc3cc2OC)CC1] | (R)-1-(4-((4-((2- fluoro-4-(3-(3- (trifluoromethyl) pyrrolidin-1- yl)phenoxy)phenyl) amino)-7- methoxyquinazolin-6- yl)amino)piperidin- 1-yl)prop-2-en-1- one |
| 321 | 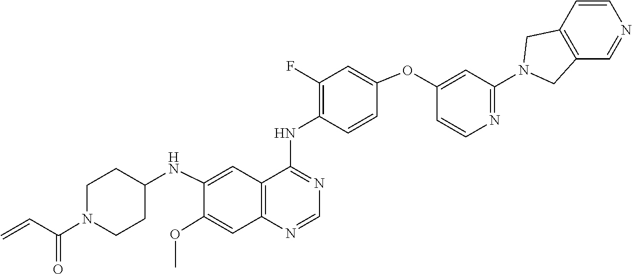 [SMILES: C=CC(=O)N1CCC(Nc2cc3c(Nc4ccc(Oc5ccnc(N6Cc7ccncc7C6)c5)cc4F)ncnc3cc2OC)CC1] | 1-(4-((4-((4-((2- (1,3-dihydro-2H- pyrrolo[3,4-c]pyridin- 2-yl)pyridin-4- yl)oxy)-2- fluorophenyl)amino)-7- methoxyquinazolin-6- yl)amino)piperidin- 1-yl)prop-2-en-1- one |
| 322 | 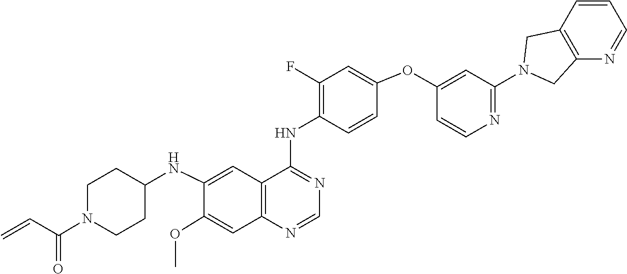 [SMILES: C=CC(=O)N1CCC(Nc2cc3c(Nc4ccc(Oc5ccnc(N6Cc7cccnc7C6)c5)cc4F)ncnc3cc2OC)CC1] | 1-(4-((4-((4-((2- (5,7-dihydro-6H- pyrrolo[3,4-b]pyridin- 6-yl)pyridin-4- yl)oxy)-2-fluorophenyl) amino)-7- methoxyquinazolin-6- yl)amino)piperidin- 1-yl)prop-2-en-1- one |
| 323 | 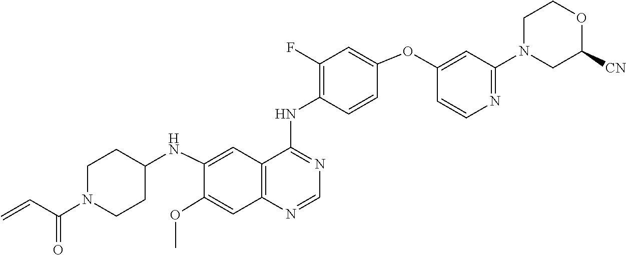 [SMILES: C=CC(=O)N1CCC(Nc2cc3c(Nc4ccc(Oc5ccnc(N6CCO[C@@H](C#N)C6)c5)cc4F)ncnc3cc2OC)CC1] | (R)-4-(4-(4-((6-((1- acryloylpiperidin- 4-yl)amino)-7- methoxyquinazolin-4- yl)amino)-3-fluorophenoxy) pyridin-2- yl)morpholine- 2-carbonitrile |
| 324 | 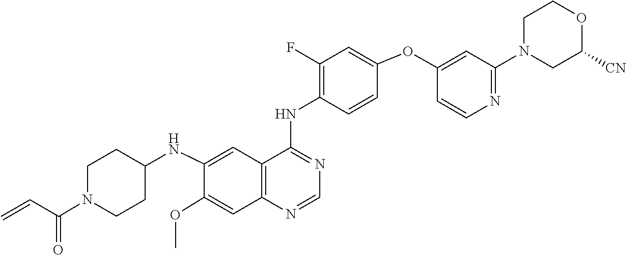 [SMILES: C=CC(=O)N1CCC(Nc2cc3c(Nc4ccc(Oc5ccnc(N6CCO[C@H](C#N)C6)c5)cc4F)ncnc3cc2OC)CC1] | (S)-4-(4-(4-((6-((1- acryloylpiperidin- 4-yl)amino)-7- methoxyquinazolin-4- yl)amino)-3-fluorophenoxy) pyridin-2- yl)morpholine- 2-carbonitrile |
| 325 | 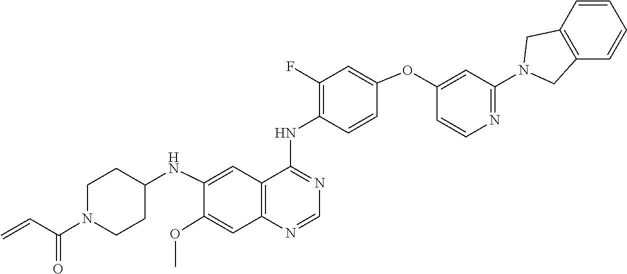 [SMILES: C=CC(=O)N1CCC(Nc2cc3c(Nc4ccc(Oc5ccnc(N6Cc7ccccc7C6)c5)cc4F)ncnc3cc2OC)CC1] | 1-(4-((4-((2-fluoro- 4-((2-(isoindolin- 2-yl)pyridin-4-yl)oxy) phenyl)amino)- 7-methoxyquinazolin-6- yl)amino)piperidin- 1-yl)prop-2-en-1- one |
| 326 | 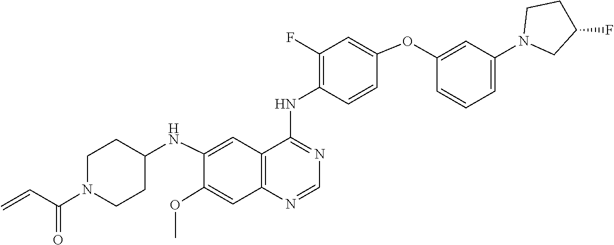 [SMILES: C=CC(=O)N1CCC(Nc2cc3c(Nc4ccc(Oc5cccc(N6CC[C@H](F)C6)c5)cc4F)ncnc3cc2OC)CC1] | (S)-1-(4-((4-((2- fluoro-4-(3-(3- fluoropyrrolidin-1- yl)phenoxy)phenyl) amino)-7- methoxyquinazolin-6- yl)amino)piperidin- 1-yl)prop-2-en-1- one |
| 327 | 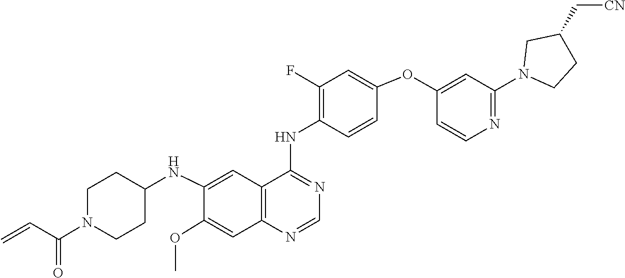 [SMILES: C=CC(=O)N1CCC(Nc2cc3c(Nc4ccc(Oc5ccnc(N6CC[C@@H](CC#N)C6)c5)cc4F)ncnc3cc2OC)CC1] | (S)-2-(1-(4-(4-((6-((1- acryloylpiperidin-4- yl)amino)-7- methoxyquinazolin- 4-yl)amino)-3- fluorophenoxy)pyridin-2- yl)pyrrolidin-3- yl)acetonitrile |
| 328 | 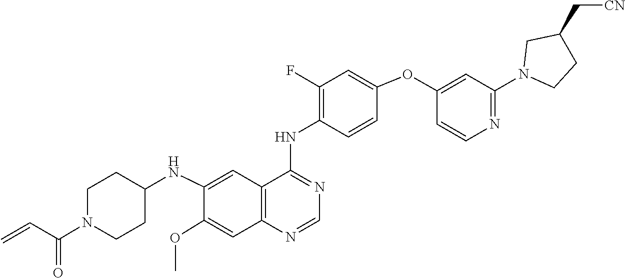 [SMILES: C=CC(=O)N1CCC(Nc2cc3c(Nc4ccc(Oc5ccnc(N6CC[C@H](CC#N)C6)c5)cc4F)ncnc3cc2OC)CC1] | (R)-2-(1-(4-(4-((6-((1- acryloylpiperidin- 4-yl)amino)-7- methoxyquinazolin- 4-yl)amino)-3- fluorophenoxy)pyridin-2- yl)pyrrolidin-3-yl) acetonitrile |
| 329 | 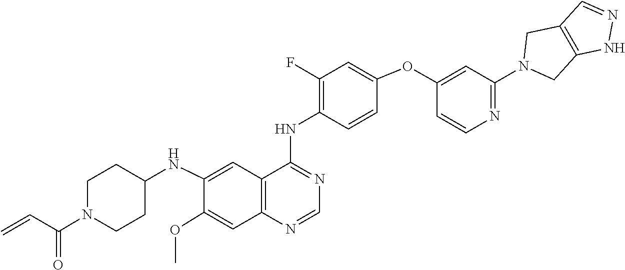 [SMILES: C=CC(=O)N1CCC(Nc2cc3c(Nc4ccc(Oc5ccnc(N6Cc7cn[nH]c7C6)c5)cc4F)ncnc3cc2OC)CC1] | 1-(4-((4-((4-((2-(4,6- dihydropyrrolo[3,4-c] pyrazol-5(1H)- yl)pyridin-4-yl)oxy)-2- fluorophenyl)amino)-7- methoxyquinazolin-6- yl)amino)piperidin- 1-yl)prop-2-en-1- one |
| 330 | 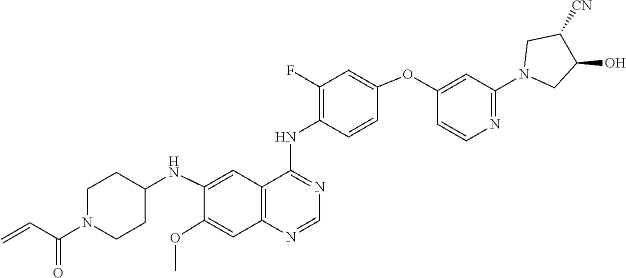 [SMILES: C=CC(=O)N1CCC(Nc2cc3c(Nc4ccc(Oc5ccnc(N6C[C@H](O)[C@@H](C#N)C6)c5)cc4F)ncnc3cc2OC)CC1] | (3R,4R)-1-(4-(4-((6-((1- acryloylpiperidin- 4-yl)amino)-7- methoxyquinazolin- 4-yl)amino)-3- fluorophenoxy) pyridin-2-yl)-4- hydroxypyrrolidine-3- carbonitrile |
| 331 | 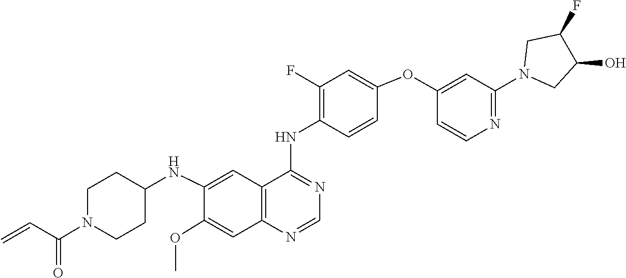 [SMILES: C=CC(=O)N1CCC(Nc2cc3c(Nc4ccc(Oc5ccnc(N6C[C@@H](F)[C@@H](O)C6)c5)cc4F)ncnc3cc2OC)CC1] | 1-(4-((4-((2-fluoro- 4-((2-((3R,4S)-3- fluoro-4- hydroxypyrrolidin-1- yl)pyridin-4-yl)oxy) phenyl)amino)-7- methoxyquinazolin-6- yl)amino)piperidin- 1-yl)prop-2-en-1- one |
| 332 | 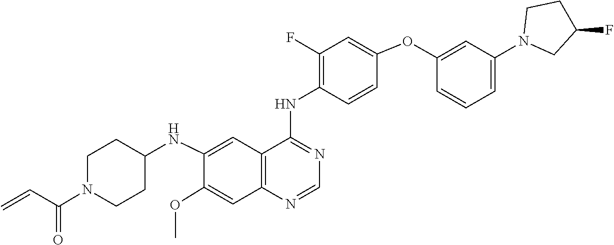 [SMILES: C=CC(=O)N1CCC(Nc2cc3c(Nc4ccc(Oc5cccc(N6CC[C@@H](F)C6)c5)cc4F)ncnc3cc2OC)CC1] | (R)-1-(4-((4-((2- fluoro-4-(3-(3- fluoropyrrolidin-1- yl)phenoxy) phenyl)amino)-7- methoxyquinazolin-6- yl)amino)piperidin- 1-yl)prop-2-en-1- one |
| 333 | 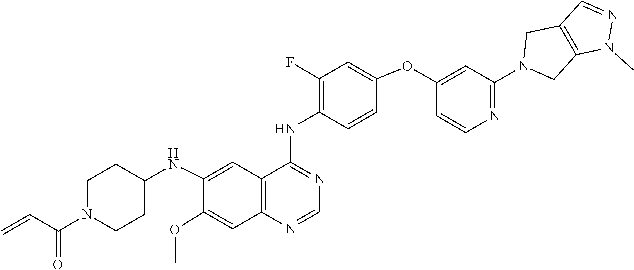 [SMILES: C=CC(=O)N1CCC(Nc2cc3c(Nc4ccc(Oc5ccnc(N6Cc7cnn(C)c7C6)c5)cc4F)ncnc3cc2OC)CC1] | 1-(4-((4-((2-fluoro- 4-((2-(1-methyl- 4,6-dihydropyrrolo [3,4-c]pyrazol- 5(1H)-yl)pyridin-4- yl)oxy)phenyl)amino)-7- methoxyquinazolin-6- yl)amino)piperidin- 1-yl)prop-2-en-1- one |
| 334 | 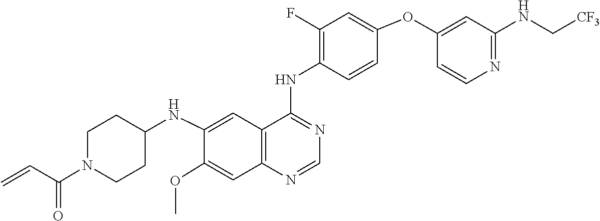 [SMILES: C=CC(=O)N1CCC(Nc2cc3c(Nc4ccc(Oc5ccnc(NCC(F)(F)F)c5)cc4F)ncnc3cc2OC)CC1] | 1-(4-((4-((2-fluoro- 4-((2-((2,2,2- trifluoroethyl)amino) pyridin-4- yl)oxy)phenyl)amino)-7- methoxyquinazolin-6- yl)amino)piperidin- 1-yl)prop-2-en-1- one |
| 335 | 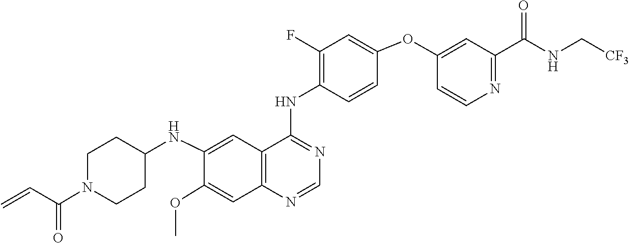 [SMILES: C=CC(=O)N1CCC(Nc2cc3c(Nc4ccc(Oc5ccnc(C(=O)NCC(F)(F)F)c5)cc4F)ncnc3cc2OC)CC1] | 4-(4-((6-((1- acryloylpiperidin-4- yl)amino)-7- methoxyquinazolin-4- yl)amino)-3- fluorophenoxy)-N- (2,2,2-trifluoroethyl) picolinamide |
| 336 | 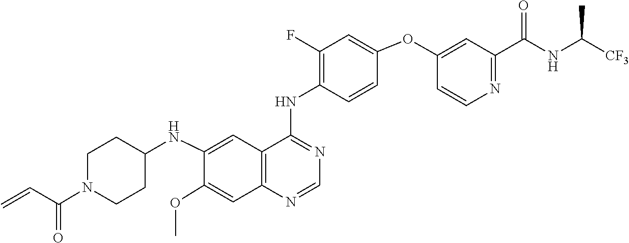 [SMILES: C=CC(=O)N1CCC(Nc2cc3c(Nc4ccc(Oc5ccnc(C(=O)N[C@@H](C)C(F)(F)F)c5)cc4F)ncnc3cc2OC)CC1] | (S)-4-(4-((6-((1- acryloylpiperidin-4- yl)amino)-7- methoxyquinazolin-4- yl)amino)-3- fluorophenoxy)-N- (1,1,1-trifluoropropan-2- yl)picolinamide |
| 337 | 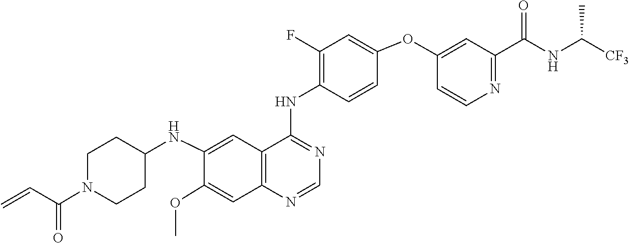 [SMILES: C=CC(=O)N1CCC(Nc2cc3c(Nc4ccc(Oc5ccnc(C(=O)N[C@H](C)C(F)(F)F)c5)cc4F)ncnc3cc2OC)CC1] | (R)-4-(4-((6-((1- acryloylpiperidin-4- yl)amino)-7- methoxyquinazolin-4- yl)amino)-3- fluorophenoxy)-N- (1,1,1-trifluoropropan-2- yl)picolinamide |
| 338 | 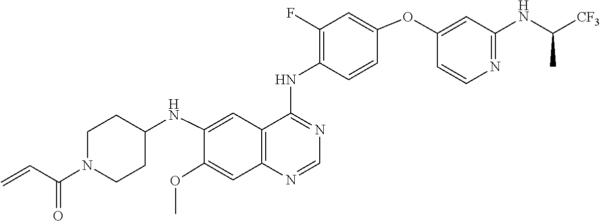 [SMILES: C=CC(=O)N1CCC(Nc2cc3c(Nc4ccc(Oc5ccnc(N[C@H](C)C(F)(F)F)c5)cc4F)ncnc3cc2OC)CC1] | (R)-1-(4-((4-((2- fluoro-4-((2-((1,1,1- trifluoropropan-2-yl) amino)pyridin-4- yl)oxy)phenyl)amino)-7- methoxyquinazolin-6- yl)amino)piperidin- 1-yl)prop-2-en-1- one |
| 339 | 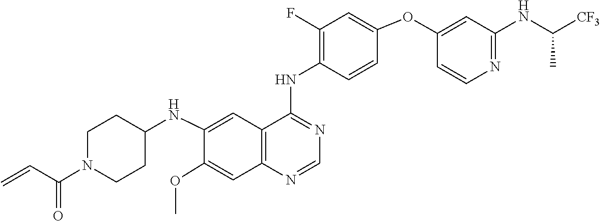 [SMILES: C=CC(=O)N1CCC(Nc2cc3c(Nc4ccc(Oc5ccnc(N[C@@H](C)C(F)(F)F)c5)cc4F)ncnc3cc2OC)CC1] | (S)-1-(4-((4-((2- fluoro-4-((2-((1,1,1- trifluoropropan-2-yl) amino)pyridin-4- yl)oxy)phenyl)amino)-7- methoxyquinazolin-6- yl)amino)piperidin- 1-yl)prop-2-en-1- one |
| 340 | 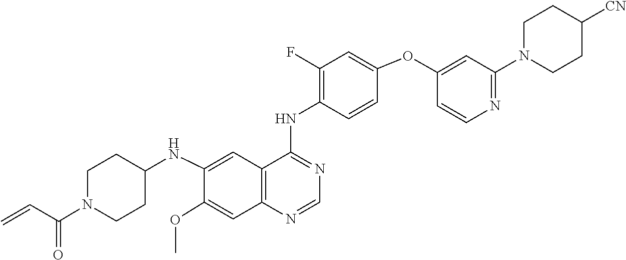 [SMILES: C=CC(=O)N1CCC(Nc2cc3c(Nc4ccc(Oc5ccnc(N6CCC(C#N)CC6)c5)cc4F)ncnc3cc2OC)CC1] | 1-(4-(4-((6-((1- acryloylpiperidin-4- yl)amino)-7- methoxyquinazolin-4- yl)amino)-3-fluorophenoxy) pyridin-2- yl)piperidine-4- carbonitrile |
| 341 | 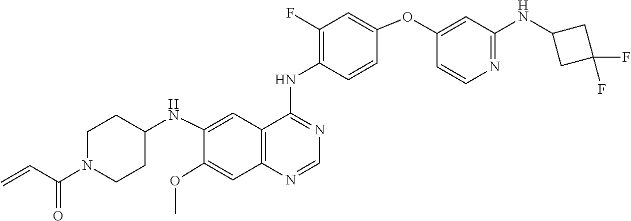 [SMILES: C=CC(=O)N1CCC(Nc2cc3c(Nc4ccc(Oc5ccnc(NC6CC(F)(F)C6)c5)cc4F)ncnc3cc2OC)CC1] | 1-(4-((4-((4-((2-((3,3- difluorocyclobutyl) amino)pyridin-4- yl)oxy)-2- fluorophenyl)amino)-7- methoxyquinazolin-6- yl)amino)piperidin- 1-yl)prop-2-en-1- one |
| 342 | 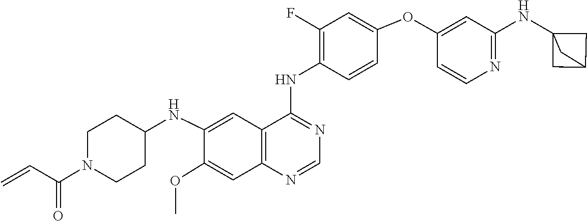 [SMILES: C=CC(=O)N1CCC(Nc2cc3c(Nc4ccc(Oc5ccnc(NC67CC(C6)C7)c5)cc4F)ncnc3cc2OC)CC1] | 1-(4-((4-((4-((2- (bicyclo[1.1.1]pentan-1- ylamino)pyridin- 4-yl)oxy)-2- fluorophenyl)amino)-7- methoxyquinazolin-6- yl)amino)piperidin- 1-yl)prop-2-en-1- one |
| 343 | 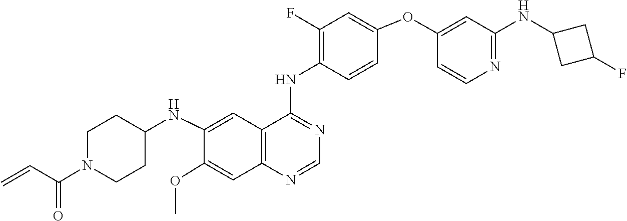 [SMILES: C=CC(=O)N1CCC(Nc2cc3c(Nc4ccc(Oc5ccnc(NC6CC(F)C6)c5)cc4F)ncnc3cc2OC)CC1] | 1-(4-((4-((2-fluoro-4-((2-((3- fluorocyclobutyl) amino)pyridin-4- yl)oxy)phenyl)amino)-7- methoxyquinazolin-6- yl)amino)piperidin- 1-yl)prop-2-en-1- one |
| 344 | 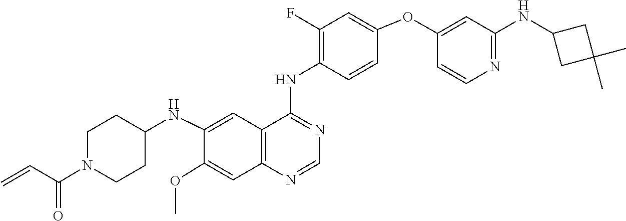 [SMILES: C=CC(=O)N1CCC(Nc2cc3c(Nc4ccc(Oc5ccnc(NC6CC(C)(C)C6)c5)cc4F)ncnc3cc2OC)CC1] | 1-(4-((4-((4-((2-((3,3- dimethylcyclobutyl) amino)pyridin-4- yl)oxy)-2- fluorophenyl)amino)-7- methoxyquinazolin-6- yl)amino)piperidin- 1-yl)prop-2-en-1- one |
| 345 | 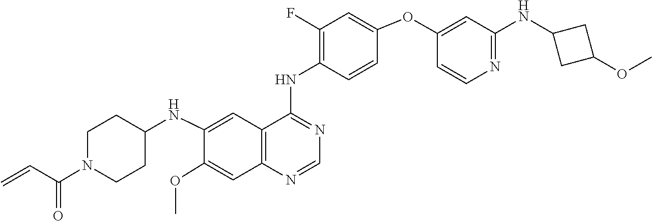 [SMILES: C=CC(=O)N1CCC(Nc2cc3c(Nc4ccc(Oc5ccnc(NC6CC(OC)C6)c5)cc4F)ncnc3cc2OC)CC1] | 1-(4-((4-((2-fluoro-4-((2-((3- methoxycyclobutyl) amino)pyridin-4- yl)oxy)phenyl)amino)-7- methoxyquinazolin-6- yl)amino)piperidin- 1-yl)prop-2-en-1- one |
| 346 | 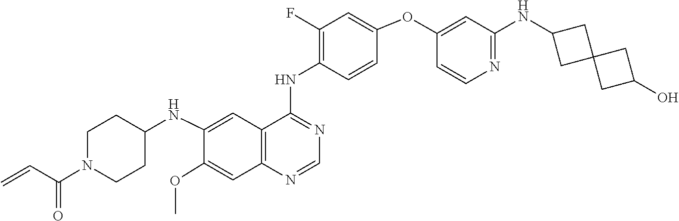 [SMILES: C=CC(=O)N1CCC(Nc2cc3c(Nc4ccc(Oc5ccnc(NC6CC7(CC(O)C7)C6)c5)cc4F)ncnc3cc2OC)CC1] | 1-(4-((4-((2-fluoro-4-((2-((6- hydroxyspiro[3.3]heptan-2- yl)amino)pyridin-4- yl)oxy)phenyl)amino)-7- methoxyquinazolin-6- yl)amino)piperidin- 1-yl)prop-2-en-1- one |
| 347 | 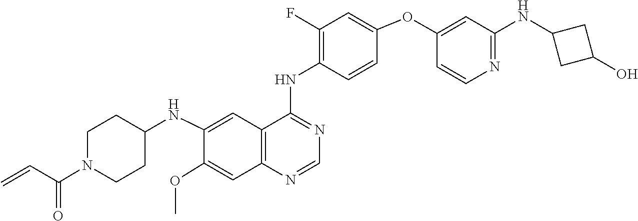 [SMILES: C=CC(=O)N1CCC(Nc2cc3c(Nc4ccc(Oc5ccnc(NC6CC(O)C6)c5)cc4F)ncnc3cc2OC)CC1] | 1-(4-((4-((2-fluoro-4-((2-((3- hydroxycyclobutyl) amino)pyridin-4- yl)oxy)phenyl)amino)-7- methoxyquinazolin-6- yl)amino)piperidin- 1-yl)prop-2-en-1- one |
| 348 | 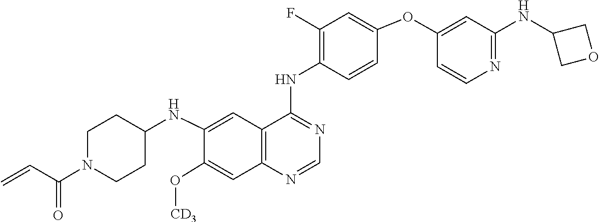 [SMILES: [2H]C([2H])([2H])Oc1cc2ncnc(Nc3ccc(Oc4ccnc(NC5COC5)c4)cc3F)c2cc1NC1CCN(C(=O)C=C)CC1] | 1-(4-((4-((2-fluoro- 4-((2-(oxetan-3- ylamino)pyridin-4- yl)oxy)phenyl)amino)- 7-(methoxy- d3)quinazolin-6-yl) amino)piperidin- 1-yl)prop-2-en-1-one |
| 349 | 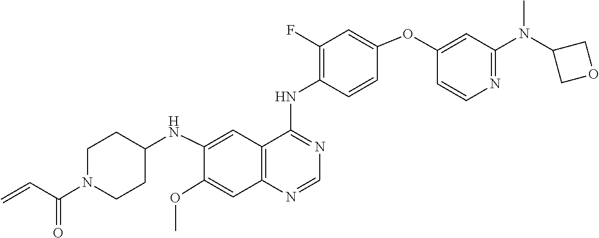 [SMILES: C=CC(=O)N1CCC(Nc2cc3c(Nc4ccc(Oc5ccnc(N(C)C6COC6)c5)cc4F)ncnc3cc2OC)CC1] | 1-(4-((4-((2-fluoro-4-((2- (methyl(oxetan-3-yl) amino)pyridin-4- yl)oxy)phenyl)amino)-7- methoxyquinazolin-6- yl)amino)piperidin- 1-yl)prop-2-en-1- one |
| 350 | 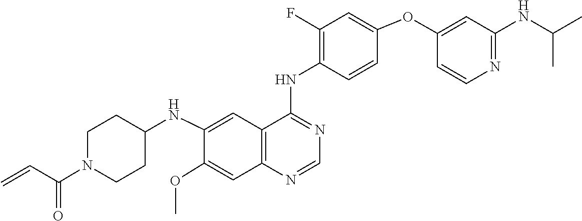 [SMILES: C=CC(=O)N1CCC(Nc2cc3c(Nc4ccc(Oc5ccnc(NC(C)C)c5)cc4F)ncnc3cc2OC)CC1] | 1-(4-((4-((2-fluoro-4-((2- (isopropylamino)pyridin-4- yl)oxy)phenyl)amino)-7- methoxyquinazolin-6- yl)amino)piperidin- 1-yl)prop-2-en-1- one |
| 351 | 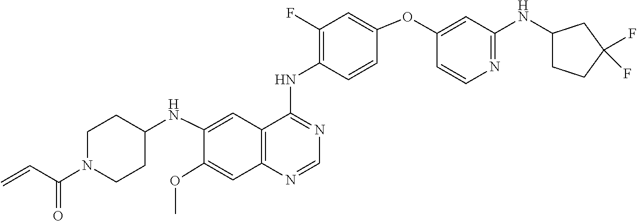 [SMILES: C=CC(=O)N1CCC(Nc2cc3c(Nc4ccc(Oc5ccnc(NC6CCC(F)(F)C6)c5)cc4F)ncnc3cc2OC)CC1] | 1-(4-((4-((4-((2-((3,3- difluorocyclopentyl) amino)pyridin-4- yl)oxy)-2- fluorophenyl)amino)-7- methoxyquinazolin-6- yl)amino)piperidin- 1-yl)prop-2-en-1- one |
| 352 | 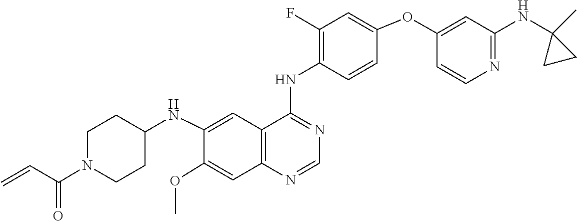 [SMILES: C=CC(=O)N1CCC(Nc2cc3c(Nc4ccc(Oc5ccnc(NC6(C)CC6)c5)cc4F)ncnc3cc2OC)CC1] | 1-(4-((4-((2-fluoro-4-((2-((1- methylcyclopropyl) amino)pyridin-4- yl)oxy)phenyl)amino)-7- methoxyquinazolin-6- yl)amino)piperidin- 1-yl)prop-2-en-1- one |
| 353 | 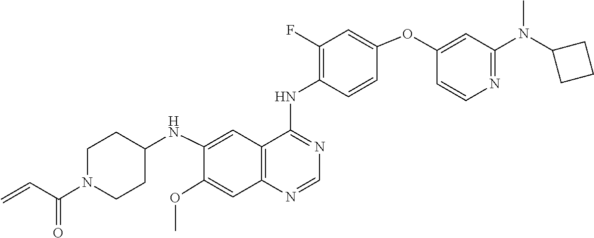 [SMILES: C=CC(=O)N1CCC(Nc2cc3c(Nc4ccc(Oc5ccnc(N(C)C6CCC6)c5)cc4F)ncnc3cc2OC)CC1] | 1-(4-((4-((4-((2- (cyclobutyl(methyl) amino)pyridin-4- yl)oxy)-2- fluorophenyl)amino)-7- methoxyquinazolin-6- yl)amino)piperidin- 1-yl)prop-2-en-1- one |
| 354 | 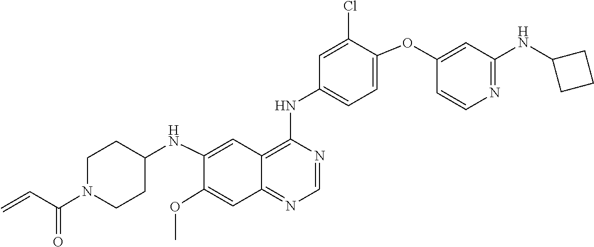 [SMILES: C=CC(=O)N1CCC(Nc2cc3c(Nc4ccc(Oc5ccnc(NC6CCC6)c5)c(Cl)c4)ncnc3cc2OC)CC1] | 1-(4-((4-((3-chloro-4-((2- (cyclobutylamino)pyridin-4- yl)oxy)phenyl)amino)-7- methoxyquinazolin-6- yl)amino)piperidin- 1-yl)prop-2-en-1- one |
| 355 | 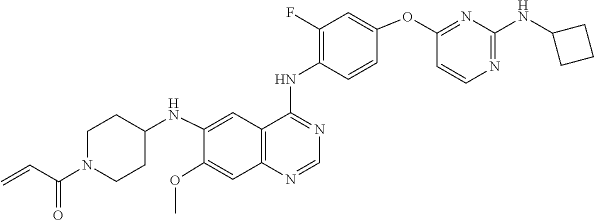 [SMILES: C=CC(=O)N1CCC(Nc2cc3c(Nc4ccc(Oc5ccnc(NC6CCC6)n5)cc4F)ncnc3cc2OC)CC1] | 1-(4-((4-((4-((2- (cyclobutylamino) pyrimidin-4- yl)oxy)-2- fluorophenyl)amino)-7- methoxyquinazolin-6- yl)amino)piperidin- 1-yl)prop-2-en-1- one |
| 356 | 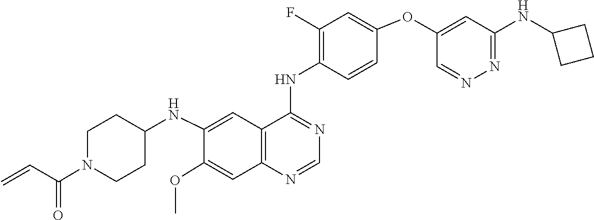 [SMILES: C=CC(=O)N1CCC(Nc2cc3c(Nc4ccc(Oc5cnnc(NC6CCC6)c5)cc4F)ncnc3cc2OC)CC1] | 1-(4-((4-((4-((6- (cyclobutylamino) pyridazin-4- yl)oxy)-2- fluorophenyl)amino)-7- methoxyquinazolin-6- yl)amino)piperidin-1- yl)prop-2-en-1- one |
| 357 | 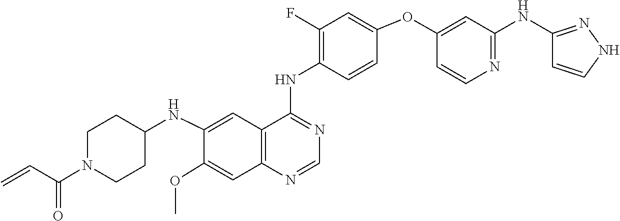 [SMILES: C=CC(=O)N1CCC(Nc2cc3c(Nc4ccc(Oc5ccnc(Nc6cc[nH]n6)c5)cc4F)ncnc3cc2OC)CC1] | 1-(4-((4-((4-((2- ((1H-pyrazol-3- yl)amino)pyridin- 4-yl)oxy)-2- fluorophenyl)amino)-7- methoxyquinazolin-6- yl)amino)piperidin- 1-yl)prop-2-en-1- one |
| 358 | 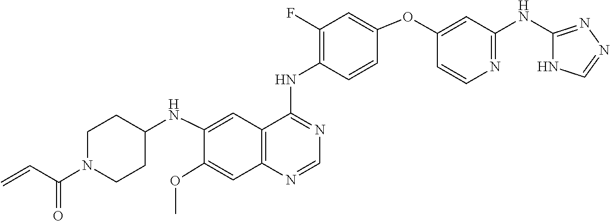 [SMILES: C=CC(=O)N1CCC(Nc2cc3c(Nc4ccc(Oc5ccnc(Nc6nnc[nH]6)c5)cc4F)ncnc3cc2OC)CC1] | 1-(4-((4-((4-((2- ((4H-1,2,4-triazol-3- yl)amino)pyridin- 4-yl)oxy)-2- fluorophenyl)amino)-7- methoxyquinazolin-6- yl)amino)piperidin- 1-yl)prop-2-en-1- one |
| 359 | 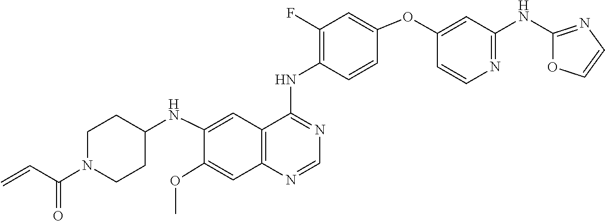 [SMILES: C=CC(=O)N1CCC(Nc2cc3c(Nc4ccc(Oc5ccnc(Nc6ncco6)c5)cc4F)ncnc3cc2OC)CC1] | 1-(4-((4-((2-fluoro- 4-((2-(oxazol-2- ylamino)pyridin-4- yl)oxy)phenyl)amino)-7- methoxyquinazolin-6- yl)amino)piperidin- 1-yl)prop-2-en-1- one |
| 360 | 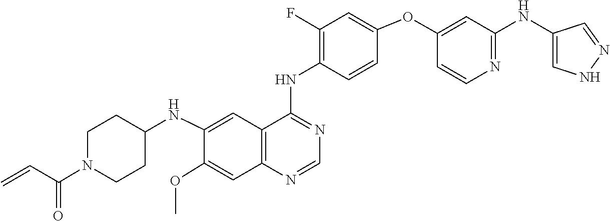 [SMILES: C=CC(=O)N1CCC(Nc2cc3c(Nc4ccc(Oc5ccnc(Nc6cn[nH]c6)c5)cc4F)ncnc3cc2OC)CC1] | 1-(4-((4-((4-((2- ((1H-pyrazol-4- yl)amino)pyridin- 4-yl)oxy)-2- fluorophenyl)amino)-7- methoxyquinazolin-6- yl)amino)piperidin- 1-yl)prop-2-en-1- one |
| 361 | 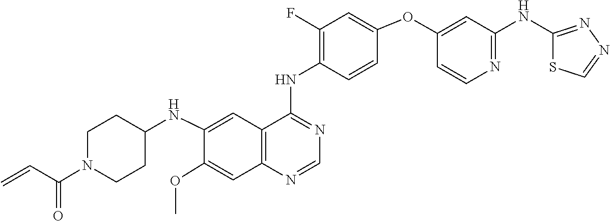 [SMILES: C=CC(=O)N1CCC(Nc2cc3c(Nc4ccc(Oc5ccnc(Nc6nncs6)c5)cc4F)ncnc3cc2OC)CC1] | 1-(4-((4-((4-((2- ((1,3,4-thiadiazol-2- yl)amino)pyridin- 4-yl)oxy)-2- fluorophenyl)amino)-7- methoxyquinazolin-6- yl)amino)piperidin- 1-yl)prop-2-en-1- one |
| 362 | 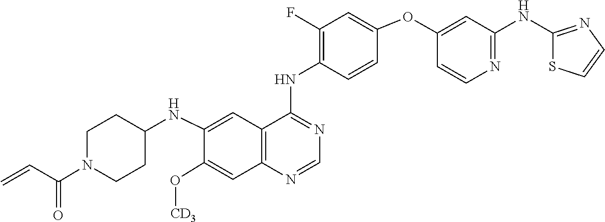 [SMILES: [2H]C([2H])([2H])Oc1cc2ncnc(Nc3ccc(Oc4ccnc(Nc5nccs5)c4)cc3F)c2cc1NC1CCN(C(=O)C=C)CC1] | 1-(4-((4-((2-fluoro- 4-((2-(thiazol-2- ylamino)pyridin-4- yl)oxy)phenyl)amino)- 7-(methoxy- d3)quinazolin-6-yl) amino)piperidin- 1-yl)prop-2-en-1-one |

### Pharmaceutical Compositions

Pharmaceutical compositions of the present disclosure comprise at least one compound of Formula (I), or tautomer, stereoisomer or a mixture of stereoisomers, or a pharmaceutically acceptable salt, or hydrate, or deuterated derivative thereof formulated together with one or more pharmaceutically acceptable carriers. These formulations include those suitable for oral, rectal, topical, buccal and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one compound of the present disclosure, which may be optionally combined with one or more accessory ingredients. Compressed tablets may be prepared by compressing, in a suitable machine, at least one compound of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one compound of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one compound of Formula (I), or a tautomer, stereoisomer or a mixture of stereoisomers, or a pharmaceutically acceptable salt, or hydrate, or deuterated derivative thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood.

Injectable compositions according to the present disclosure may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula (I), or a tautomer, stereoisomer or a mixture of stereoisomers, or a pharmaceutically acceptable salt, or hydrate, or deuterated derivative thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 2
Equivalent Surface Area Dosage Factors.

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

The present disclosure provides a compound of Formula (I), or a tautomer, stereoisomer or a mixture of stereoisomers, or a pharmaceutically acceptable salt, or hydrate, or deuterated derivative thereof, to be administered to treat cancer in a subject in need thereof.

In some embodiments, the cancer is associated with an EGFR or HER2 exon insertion mutation.

In some embodiments, the cancer is selected from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

In some embodiments, the cancer is lung cancer. In a further embodiment, the cancer is non-small cell lung cancer. In some embodiments, the cancer is pancreatic cancer. In some embodiments, the cancer is colon cancer. In some embodiments, the cancer is breast cancer.

In some embodiments, a compound of Formula (I), or a tautomer, stereoisomer or a mixture of stereoisomers, or a pharmaceutically acceptable salt, or hydrate, or deuterated derivative thereof, is administered as a pharmaceutical composition.

The concentration and route of administration to the patient will vary depending on the cancer to be treated.

In one embodiment, a compound of Formula (I), or a tautomer, stereoisomer or a mixture of stereoisomers, or a pharmaceutically acceptable salt, or hydrate, or deuterated derivative thereof, is administered in combination with another therapeutic agent, e.g., chemotherapy, or used in combination with other treatments, such as radiation or surgical intervention, either as an adjuvant prior to surgery or post-operatively. In some embodiments, the therapeutic agent is chosen from gemcitabine, cisplatin, erlotinib, gefitinib, pemetrexed, bevacizumab, cetuximab, trastuzumab, pertuzumab, sorafenib, lapatinib, cobimetinib, selumetinib, and everolimus.

Also provided herein is a compound of Formula (I), or a tautomer, stereoisomer or a mixture of stereoisomers, or a pharmaceutically acceptable salt, or hydrate, or deuterated derivative thereof, or a pharmaceutical composition thereof as defined herein for use in therapy.

Also provided herein is a compound of Formula (I), or a tautomer, stereoisomer or a mixture of stereoisomers, or a pharmaceutically acceptable salt, or hydrate, or deuterated derivative thereof, or a pharmaceutical composition thereof as defined herein for use in the treatment of cancer.

Also provided herein is a compound of Formula (I), or a tautomer, stereoisomer or a mixture of stereoisomers, or a pharmaceutically acceptable salt, or hydrate, or deuterated derivative thereof, for use in the inhibition of EGFR.

Also provided herein is a compound of Formula (I), or a tautomer, stereoisomer or a mixture of stereoisomers, or a pharmaceutically acceptable salt, or hydrate, or deuterated derivative thereof, or a pharmaceutical composition thereof as defined herein, for use in the treatment of a disease or disorder associated with an EGFR or HER2 exon 20 insertion mutation.

Also provided herein is the use of a compound of Formula (I), or a tautomer, stereoisomer or a mixture of stereoisomers, or a pharmaceutically acceptable salt, or hydrate, or deuterated derivative thereof, as defined herein in the manufacture of a medicament for the treatment of cancer.

Also provided herein is a use of a compound of Formula (I), or a tautomer, stereoisomer or a mixture of stereoisomers, or a pharmaceutically acceptable salt, or hydrate, or deuterated derivative thereof, as defined herein in the manufacture of a medicament for the inhibition of activity of EGFR.

Also provided herein is the use of a compound of Formula (I), or a tautomer, stereoisomer or a mixture of stereoisomers, or a pharmaceutically acceptable salt, or hydrate, or deuterated derivative thereof, as defined herein, in the manufacture of a medicament for the treatment of a disease or disorder associated with an EGFR or HER2 exon 20 insertion mutation.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The invention also provides for a method of inhibiting EGFR kinase activity in a cell comprising contacting the cell with an effective amount of an EGFR antagonist. In one embodiment, the administered amount is a therapeutically effective amount and the inhibition of EGFR kinase activity further results in the inhibition of the growth of the cell. In a further embodiment, the cell is a cancer cell.

Inhibition of cell proliferation is measured using methods known to those skilled in the art. For example, a convenient assay for measuring cell proliferation is the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high-throughput screening (HTS). See Cree et al (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU). Inhibition of cell proliferation may also be measured using colony formation assays known in the art.

Furthermore, the invention provides for methods of treating a condition associated with an EGFR or HER2 exon 20 insertion mutation in a subject suffering therefrom, comprising administering to the subject a therapeutically effective amount of an EGFR antagonist. In one embodiment, the condition is a cell proliferative disease.

Treatment of the cell proliferative disorder by administration of an EGFR antagonist results in an observable and/or measurable reduction in or absence of one or more of the following: reduction in the number of cancer cells or absence of the cancer cells; reduction in the tumor size; inhibition of cancer cell infiltration into peripheral organs including the spread of cancer into soft tissue and bone; inhibition of tumor metastasis; inhibition, to some extent, of tumor growth; and/or relief to some extent, one or more of the symptoms associated with the specific cancer; reduced morbidity and mortality, and improvement in quality of life issues. To the extent the EGFR antagonist may prevent growth and/or kill existing cancer cells, it may be cytostatic and/or cytotoxic. Reduction of these signs or symptoms may also be felt by the patient.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about −10° C. to about 200° C. Further, except as otherwise specified, reaction times and conditions are intended to be approximate, e.g., taking place at about atmospheric pressure within a temperature range of about −10° C. to about 200° C. over a period that can be, for example, about 1 to about 24 hours; reactions left to run overnight in some embodiments can average a period of about 16 hours.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3^{rd} Ed., 1990 New York: Plenum Press; Mundy et al., Name Reaction and Reagents in Organic Synthesis, 2^{nd} Ed., 2005 Hoboken, NJ: J. Wiley & Sons. Specific illustrations of suitable separation and isolation procedures are given by reference to the examples hereinbelow. However, other equivalent separation or isolation procedures can also be used.

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3^{rd} Ed., John Wiley & Sons). These groups may be removed at a convenient stage of the compound synthesis using methods that are readily apparent to those skilled in the art.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein.

The skilled artisan will understand that standard atom valencies apply to all compounds disclosed herein in genus or named compound for unless otherwise specified.

The following abbreviations have the definitions set forth below:
ACN acetonitrile
(Boc)₂O Di-tert-butyl dicarbonate
DCM dichloromethane
DIEA diisopropylethylamine
DMF dimethylformamide
EA ethyl acetate
HCl hydrochloric acid
Pd₂(dba)₃ Tris(dibenzylideneacetone)dipalladium(0)
LC/MS liquid chromatography/mass spectrometry
NMR nuclear magnetic resonance
S-Phos 2-Dicyclohexylphosphino-2′,6′-dimethoxybiphenyl
TEA triethylamine
TFA trifluoroacetic acid
THE tetrahydrofuran
TsOH p-toluenesulfonic acid

### General Synthetic Schemes

The disclosed compounds of Formula I can be prepared according to the following schemes. The following schemes represent the general methods used in preparing these compounds. However, the synthesis of these compounds is not limited to these representative methods, as they can also be prepared through various other methods by those skilled in the art of synthetic chemistry. Unless noted otherwise, X, Y, R₁, R₂ and R₃, when present in Schemes A-F below, are as defined in Formula 1.

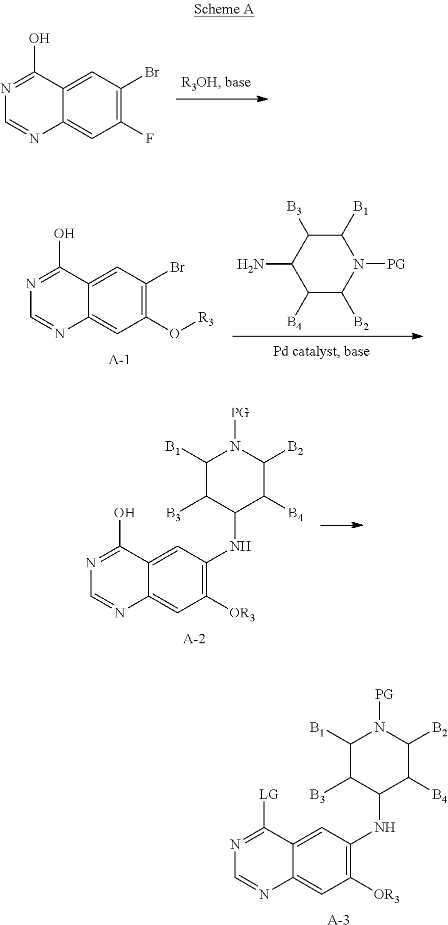

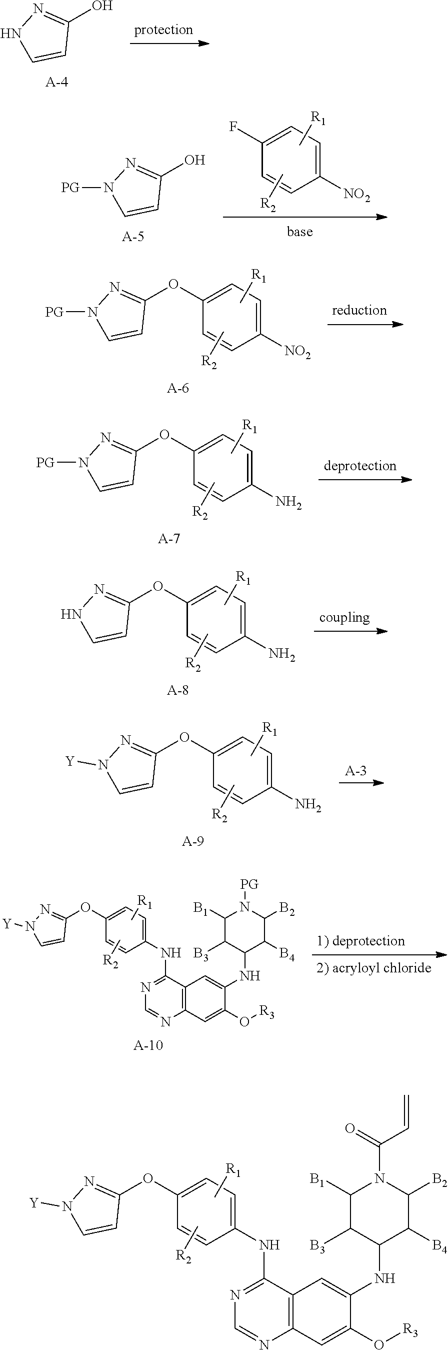

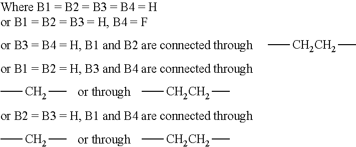

In Scheme A, 6-bromo-7-fluoroquinazolin-4-ol can react with an alkoxide generated from an alcohol R₃OH with a base such as NaH or KO^{t}Bu to produce intermediate A-1, which can undergo a coupling reaction with N-protected 4-aminopiperidine or substituted 4-aminopoperidine with a suitable palladium catalyst (PG=protective group). The hydroxyl group of intermediate A-2 can be converted to a suitable leaving group (LG) such as chloro, triflate or tosylate to give an intermediate A-3. The pyrazole NH group of 1H-pyrazol-3-ol can be protected by a suitable protecting group such as Boc. The hydroxyl group of intermediate A-5 can displace the fluorine of optionally substituted para-fluoro nitrobenzene with a base such as K₂CO₃ or Cs₂CO₃ to give an intermediate A-6. The nitro group of intermediate A-6 can be reduced to amino under the conditions such as Pd/C, H₂, Fe/NH₄Cl or sodium dithionite to provide A-7. After the deprotection, the pyrazole NH group of intermediate A-8 can undergo a coupling reaction with heteroaryl halides or heteroaryl boronic acids to provide intermediate A-9 where Y is an aromatic or heteroaromatic ring. The aniline intermediate A-9 can undergo a substitution reaction with intermediate A-3 to generate intermediate A-10. Deprotection of the piperidine protecting group following by amide formation with acryloyl chloride can provide target compounds represented by Formula I.

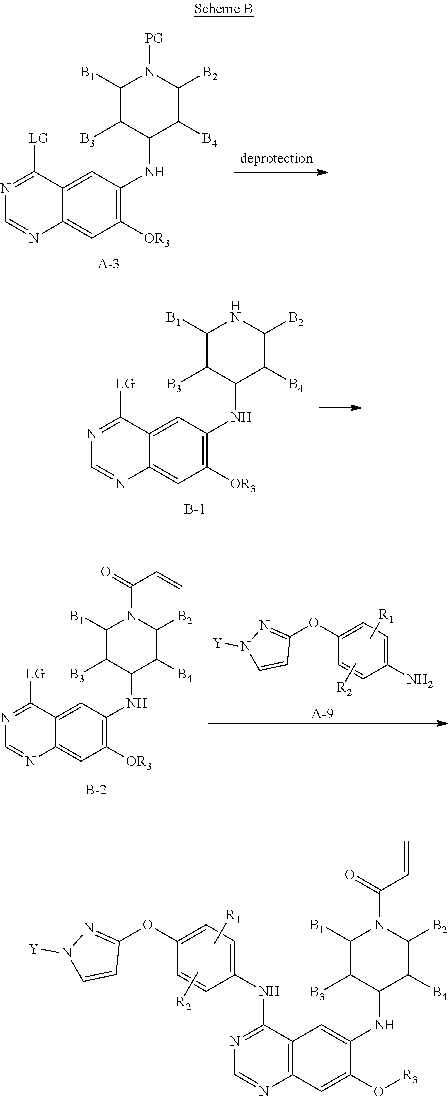

Alternatively, as described in Scheme B, deprotection of intermediate A-3 followed by amide formation can give intermediate B-2. Substitution of the leaving group (LG) in B-2 with aniline intermediate A-9 can also provide the desired final product.

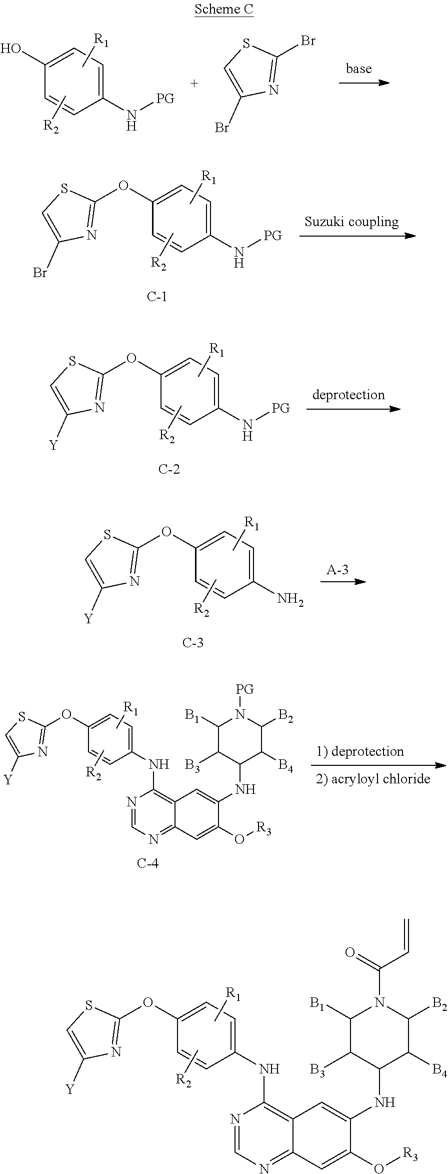

In Scheme C, 2,4-dibromothiazole can react with N-protected para-aminophenol in the presence of a base such as K₂CO₃ or Cs₂CO₃ to give intermediate C-1. Suzuki coupling of bromo intermediate C-1 with a heteroaryl boronic acid or pinacol ester with a suitable palladium catalyst can produce intermediate C-2. After deprotection, the aniline intermediate C-3 can react with A-3 to give intermediate C-4. Thiazole derivatives of compounds represented by Formula I can be prepared after deprotection of nitrogen protecting group followed by amide formation using acryloyl chloride.

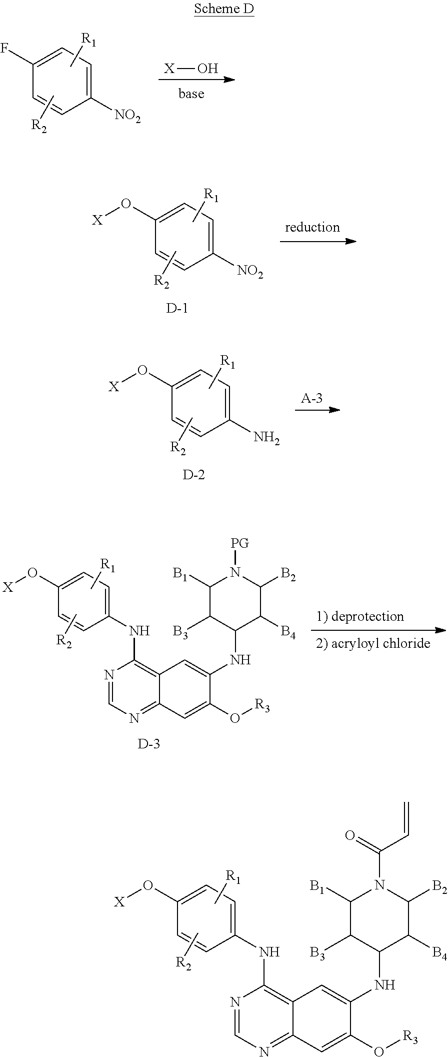

As shown in Scheme D, an optionally substituted para-fluoro nitrobenzene can react with a heteroaryl alcohol in the presence of a strong base such as NaH or KO^{t}Bu. After reduction of nitro to an amino group, the aniline intermediate D-2 can be used to prepare the target compound represented by Formula I by using the same reaction sequences as described in Scheme A.

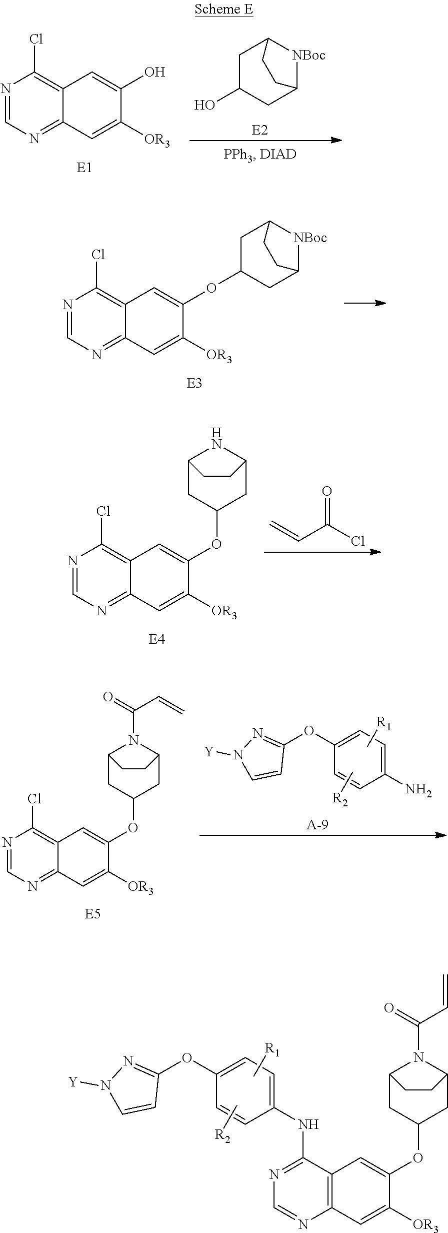

As described in Scheme E, Mitsunobu reaction between E1 and E2 can form an ether E3, which can be deprotected to provide E4. The amide formation of E4 with acryloyl chloride can give E5, which can be converted to the desired structure of Formula I where Q is oxygen.

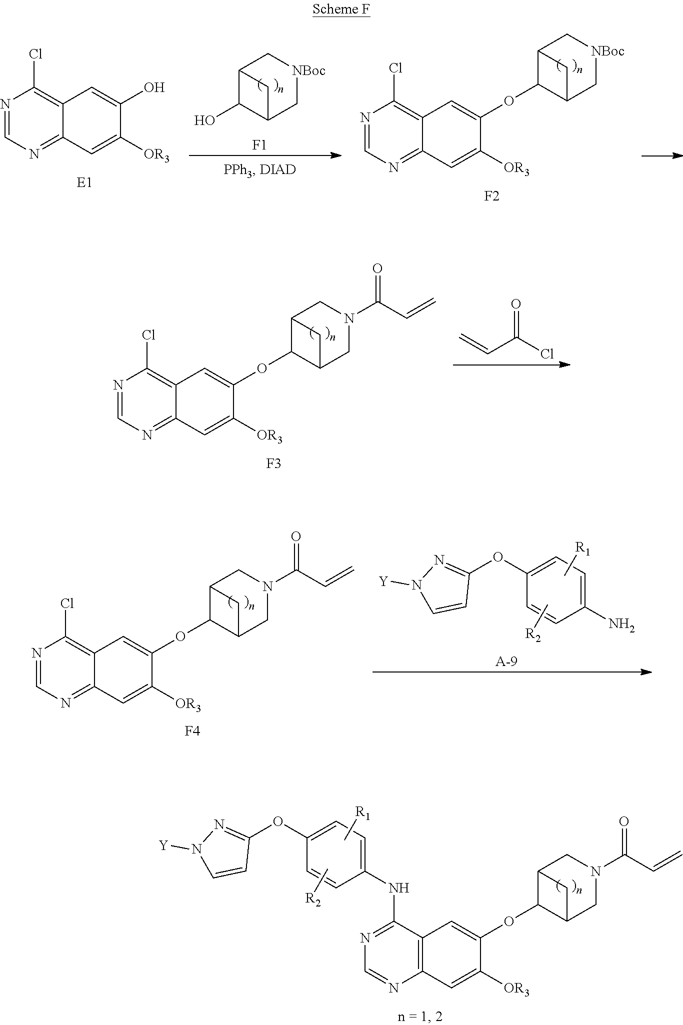

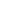

Scheme F describes the synthesis of Formula I where Q is oxygen. Intermediate F2, F3 and F4 can be prepared using the synthetic sequence as shown in Scheme E.

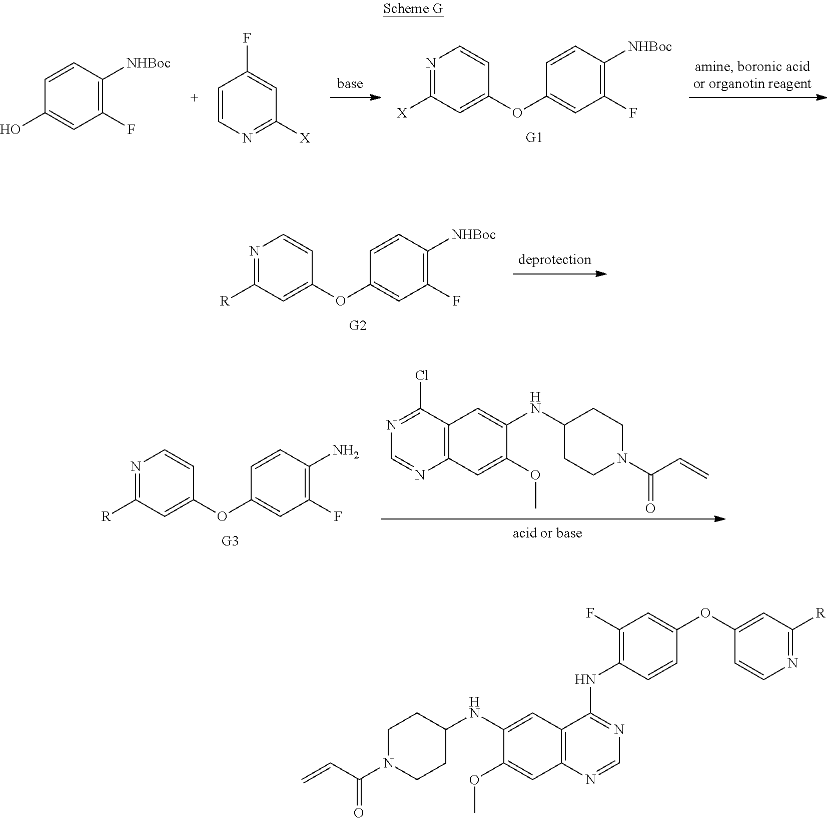

In Scheme G, tert-butyl (2-fluoro-4-hydroxyphenyl)carbamate can undergo substitution reaction with suitably substituted 4-fluoropyridine with X being halogen, such as Br, Cl or ester, aldehyde, cyano, nitro group. Intermediate G1 can react with amines assisted with a base or metal catalyst, or with boronic acids and organotin reagents in Suzuki or Stille coupling reactions to give intermediate G2. Removal of the Boc protecting group under acidic conditions gives intermediate G3. In case X is an ester or cyano group, intermediate G1 can be converted to amides or heterocycles (see R group, such as oxadiazoles, tetrazoles) with conditions known to a person of skill in the art. In case of X is aldehyde, intermediate G1 can undergo reductive amination with amines. In case of X is nitro group, the nitro group can be reduced to amino group which can undergo further functional group transformation known to one of skill in the art. In the first step of Scheme G, other regioisomers of fluoropyridines bearing a substituent of X to produce regioisomer of G1 intermediate. Alternatively, other heteroaryls such as pyrimidine, pyrazine, pyridazine or triazine with a leaving group such as halide, tosylate, triflate, or methylsulfone and a suitable substituent X can be used in step 1 to produce intermediate G1 with a pyrimidine, pyrazine, pyridazine or triazine ring instead of a pyridine ring.

### Example 1: Preparation of 1-(4-((4-((2-fluoro-4-((1-(2-methoxypyrimidin-5-yl)-1H-pyrazol-3-yl)oxy)phenyl)amino)-7-methoxyquinazolin-6-yl)amino)piperidin-1-yl)prop-2-en-1-one (Compound 1)

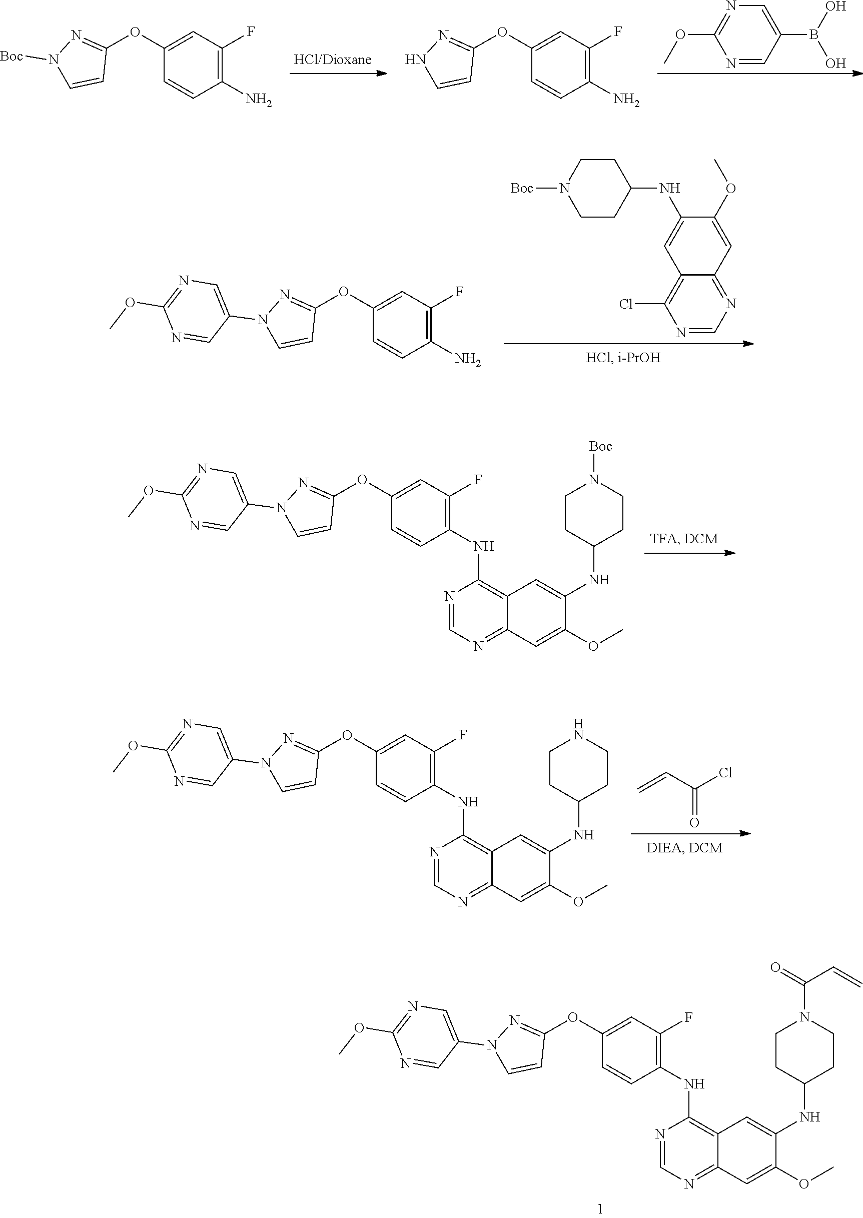

### Step 1: Preparation of 4-((1H-pyrazol-3-yl)oxy)-2-fluoroaniline

To a solution of tert-butyl 3-(4-amino-3-fluorophenoxy)-1H-pyrazole-1-carboxylate (1.5 g, 5.12 mmol, see preparation procedure in Example 8) in DCM (30 mL) was added HCl/dioxane (4.0 N, 10 mL). The reaction mixture was stirred for 2 hours at 25° C. The reaction solution was concentrated in vacuo to afford 4-((1H-pyrazol-3-yl)oxy)-2-fluoroaniline (900 mg, 91%) as a yellow solid. LC/MS: 194 [M+H]⁺.

### Step 2: Preparation of 2-fluoro-4-((1-(2-methoxypyrimidin-5-yl)-1H-pyrazol-3-yl)oxy)aniline

To a solution of 4-((1H-pyrazol-3-yl)oxy)-2-fluoroaniline (900 mg, 4.64 mmol) in DCE (30 mL mL) was added (2-methoxypyrimidin-5-yl)boronic acid (1.4 g, 9.32 mmol), Cu(AcO)₂ (2.1 g, 9.32 mmol) and pyridine (1.8 g, 23.3 mmol). The solution was stirred for 16 hours at 50° C. The reaction solution was concentrated and purified by Combi-Flash (DCM/MeOH=1 1:1) to afford 2-fluoro-4-((1-(2-methoxypyrimidin-5-yl)-1H-pyrazol-3-yl)oxy)aniline (500 mg, 36%) as a white solid. LC/MS: 302 [M+H]⁺.

### Step 3: Preparation of tert-butyl 4-((4-((2-fluoro-4-((1-(2-methoxypyrimidin-5-yl)-1H-pyrazol-3-yl)oxy)phenyl)amino)-7-methoxyquinazolin-6-yl)amino)piperidine-1-carboxylate

To a solution of 2-fluoro-4-((1-(2-methoxypyrimidin-5-yl)-1H-pyrazol-3-yl)oxy)aniline (160 mg, 0.53 mmol) in i-PrOH (15 mL) was added tert-butyl 4-((4-chloro-7-methoxyquinazolin-6-yl)amino)piperidine-1-carboxylate (208 mg, 0.53 mmol, see preparation procedure in Example 2 for the preparation of compound 3) and HCl/dioxane (4N, 0.1 mL). The reaction mixture was stirred for 16 hours at room temperature. The solution was concentrated and purified by Combi-Flash (DCM/MeOH=11/1) to afford tert-butyl 4-((4-((2-fluoro-4-((1-(2-methoxypyrimidin-5-yl)-1H-pyrazol-3-yl)oxy)phenyl)amino)-7-methoxyquinazolin-6-yl)amino)piperidine-1-carboxylate (196 mg, 56%) as a yellow solid. LC/MS: 658 [M+H]⁺.

### Step 4: Preparation of N4-(2-fluoro-4-((1-(2-methoxypyrimidin-5-yl)-1H-pyrazol-3-yl)oxy)phenyl)-7-methoxy-N6-(piperidin-4-yl)quinazoline-4,6-diamine

A solution of tert-butyl 4-((4-((2-fluoro-4-((1-(2-methoxypyrimidin-5-yl)-1H-pyrazol-3-yl)oxy)phenyl)amino)-7-methoxyquinazolin-6-yl)amino)piperidine-1-carboxylate (170 mg, 0.26 mmol) in DCM/TFA (6 mL/2 mL) was stirred for 1 hour at room temperature. The reaction mixture was poured into water (60 mL) and extracted with DCM (50 mL×3). The organic layer was combined, dried over Na₂SO₄ and concentrated in vacuo to afford N4-(2-fluoro-4-((1-(2-methoxypyrimidin-5-yl)-1H-pyrazol-3-yl)oxy)phenyl)-7-methoxy-N6-(piperidin-4-yl)quinazoline-4,6-diamine (120 mg, crude) as a gray white solid. LC/MS: 558 [M+H]⁺.

### Step 5: Preparation of 1-(4-((4-((2-fluoro-4-((1-(2-methoxypyrimidin-5-yl)-1H-pyrazol-3-yl)oxy)phenyl)amino)-7-methoxyquinazolin-6-yl)amino)piperidin-1-yl)prop-2-en-1-one (1)

To a solution of N4-(2-fluoro-4-((1-(2-methoxypyrimidin-5-yl)-1H-pyrazol-3-yl)oxy)phenyl)-7-methoxy-N6-(piperidin-4-yl)quinazoline-4,6-diamine (110 mg, 0.2 mmol) in CH2Cl2 (10 mL) was added DIEA (52 mg, 0.4 mmol) and acryloyl chloride (18 mg, 0.2 mmol) at −78° C. The solution was stirred for 3 min at −78° C. The reaction solution was concentrated and purified by Prep-HPLC [MeCN—H₂O (0.1% TFA), 40%-70%], and desired fractions were pooled and lyophilized to give the desired compound (14 mg, 12%) as a white solid. LC/MS: 612 [M+H]⁺; ¹H NMR (400 MHz, CD₃OD) δ8.95 (s, 2H), 8.50 (s, 1H), 8.26 (d, J=8.4 Hz, 1H), 7.53 (t, J=8.6 Hz, 1H), 7.23 (s, 1H), 7.12-7.20 (m, 3H), 6.85-6.83 (m, 1H), 6.28 (d, J=2.6 Hz, 1H), 6.19-6.17 (m, 1H), 5.77-5.75 (m, 1H), 4.62-4.60 (m, 1H), 4.23-4.20 (m, 1H), 4.11 (s, 3H), 4.05 (s, 3H), 3.83-3.82 (m, 1H), 3.47-3.45 (m, 1H), 3.01-2.99 (m, 1H), 2.19 (d, J=12.8 Hz, 2H), 1.56-1.53 (m, 2H).

### Example 2: Preparation of 1-(4-((4-((2-fluoro-4-((1-(6-methylpyridin-3-yl)-1H-pyrazol-3-yl)oxy)phenyl)amino)-7-methoxyquinazolin-6-yl)amino)piperidin-1-yl)prop-2-en-1-one (Compound 3)

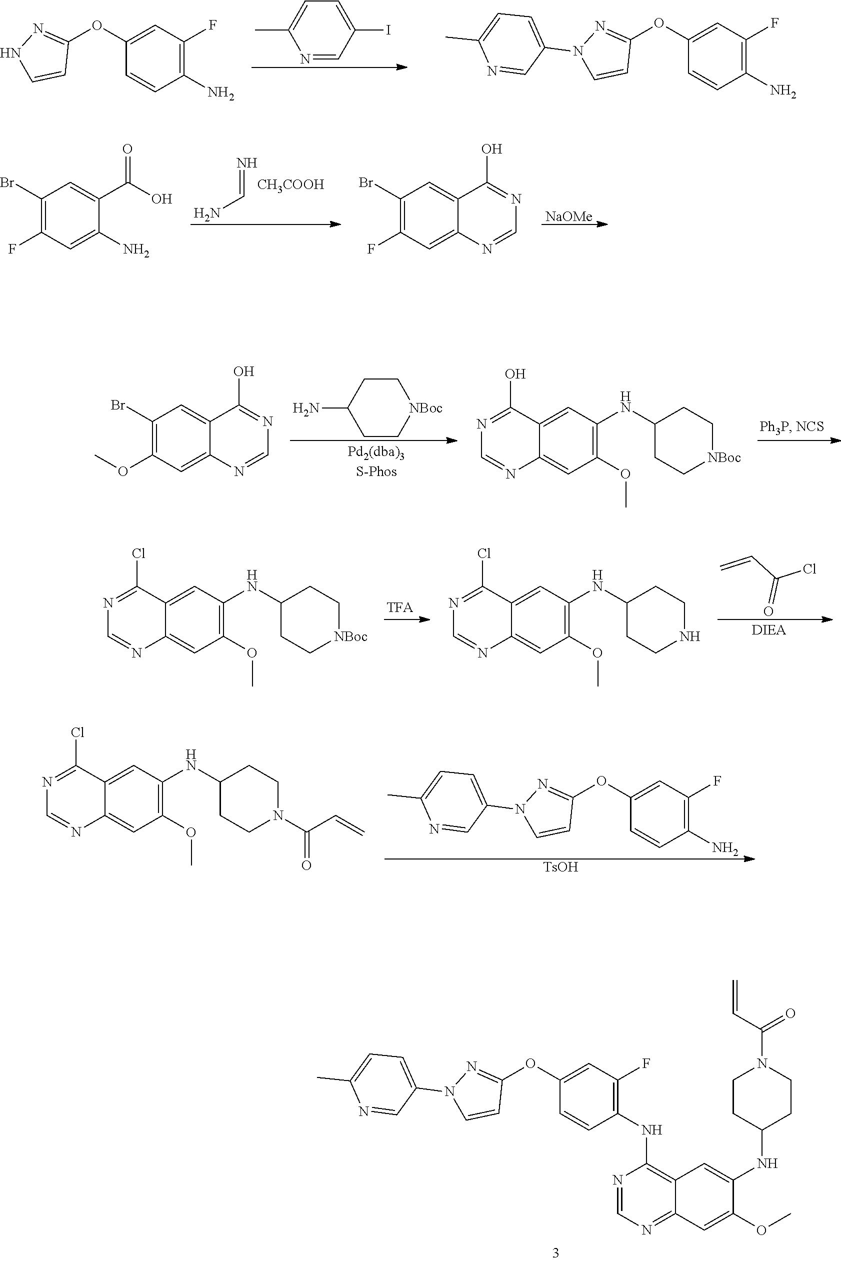

### Step 1: Preparation of 2-fluoro-4-((1-(6-methylpyridin-3-yl)-1H-pyrazol-3-yl)oxy)aniline

To a solution of 2-fluoro-4-(1H-pyrazol-3-yloxy)aniline (1 g, 5.2 mmol) in THE (20 mL) stirred at room temperature was added 5-iodo-2-methylpyridine (1.36 g, 6.2 mmol), K₃PO4 (2.2 g, 10.4 mmol), (1R,2R)-1-N,2-N-dimethylcyclohexane-1,2-diamine (1.47 g, 10.4 mmol) and CuI (986 mg, 5.2 mmol). The reaction mixture was stirred at 80° C. under Ar for 3 hours. The mixture was diluted with ethyl acetate (80 mL) and filtered through a Celite pad. The filtrate was concentrated in vacuum to give a crude product. The crude product was purified by flash column chromatography (PE/EA=1:1) to give the desired product (800 mg, yield=54.4%), LC/MS: 285.1 [M+H]⁺; ¹HNMR (400 MHz, CD₃OD) δ=8.85 (d, J=2.8 Hz, 1H), 8.45 (d, J=2.8 Hz, 1H), 7.99 (dd, J=8.4, 2.8 Hz, 1H), 7.35 (d, J=8.4 Hz, 1H), 7.02-6.93 (m, 1H), 6.82-6.74 (m, 2H), 6.05 (d, J=2.8 Hz, 1H), 5.02 (s, 2H), 2.48 (s, 3H).

### Step 2: Preparation of 6-bromo-7-fluoroquinazolin-4-ol

To a solution of 2-amino-5-bromo-4-fluorobenzoic acid (50 g, 213.7 mmol) in 2-methoxyethanol (100 mL) stirred at room temperature was added formamidine acetate (44.5 g, 427.4 mmol). The reaction mixture was stirred at 120° C. for 10 hours, then cooled to room temperature and poured into water (100 mL). After adjusting pH to 7-8 with NH₃·H₂O, the mixture was filtered and the filter cake was dried in vacuum to give the desired product (42 g, purity=90%, yield=72.8%). LC/MS: 242.8 [M+H]⁺.

### Step 3: Preparation of 6-bromo-7-methoxyquinazolin-4-ol

To a solution of sodium methoxide (28 g, 518.4 mmol) in MeOH (100 mL) stirred at room temperature was added 6-bromo-7-fluoroquinazolin-4-ol (42 g, 155.9 mmol). The reaction mixture was stirred at reflux for 12 hours. The mixture was cooled to room temperature and poured into ice/water (50 mL). After adjusting pH to 5-6 with HCl (6 N), the mixture was filtered and the filter cake was dried in vacuum to give the product (33.4 g, 70% purity, yield=58.9%). LC/MS: 254.9 [M+H]⁺.

### Step 4: Preparation of tert-butyl 4-((4-hydroxy-7-methoxyquinazolin-6-yl)amino)piperidine-1-carboxylate

The suspension containing 6-bromo-7-methoxyquinazolin-4-ol (25.7 g, 70% purity, 70.6 mmol), tert-butyl 4-amino-piperidine-1-carboxylate (21.2 g, 105.9 mmol), Pd₂(dba)₃ (6.5 g, 7.0 mmol), S-Phos (2.9 g, 7.0 mmol) and t-BuONa (20.4 g, 211.8 mmol) in toluene (200 mL) was stirred at 100° C. overnight. The mixture was filtered and the filtrate was concentrated in vacuum to give a crude material which was purified by flash column chromatography using DCM/MeOH=10:1 to afford the desired product as a white solid (15 g, 60% purity, 34.4%). LC/MS: 375.0 [M+H]⁺.

### Step 5: Preparation of tert-butyl 4-((4-chloro-7-methoxyquinazolin-6-yl)amino)piperidine-1-carboxylate

To a solution of PPh₃ (7.1 g, 27 mmol) in THE (100 mL) was added NCS (3.61 g, 27 mmol) in portions at 0° C. under argon atmosphere. The mixture was stirred at room temperature for 1 hour then tert-butyl {4-[(4-hydroxy-7-methoxyquinazolin-6-yl)amino]piperidin-1-yl} formate (5.62 g, 60% purity, 9.0 mmol) was added. The reaction mixture was stirred at 50° C. for 10 hours. The mixture was treated with TEA (5 mL) and then concentrated in vacuum. The crude material was purified by silica gel chromatography with PE/EA=1:1 to give the desired product (8 g, purity=30%, containing PPh₃O, yield=68%), LC/MS: 393.0 [M+H]⁺.

### Step 6: Preparation of 4-chloro-7-methoxy-N-(piperidin-4-yl)quinazolin-6-amine

Tert-butyl 4-((4-chloro-7-methoxyquinazolin-6-yl)amino)piperidine-1-carboxylate (8 g, 30% purity, 6.1 mmol) was stirred in TFA/DCM (1:5, 120 ml) at room temperature for 2 hours. The mixture was concentrated in vacuum to give the crude product (8 g, crude). LC/MS: 293.0 [M+H]⁺.

### Step 7: Preparation of 1-(4-((4-chloro-7-methoxyquinazolin-6-yl)amino)piperidin-1-yl)prop-2-en-1-one

To a solution of 4-chloro-7-methoxy-N-(piperidin-4-yl)quinazolin-6-amine (8 g crude, 6.1 mmol) in DCM (100 mL) was added prop-2-enoyl chloride (549 mg, 6.1 mmol) dropwise at −78° C. Then DIEA (9 g, 69.7 mmol) was added slowly. The reaction mixture was stirred at −78° C. for 1 hour and quenched by adding water (2 mL). The mixture was washed with brine, dried over Na₂SO₄ and concentrated in vacuum to give a crude material which was purified by flash chromatography (DCM/MeOH=10:1) to give the desired product (6.1 g, 25% purity containing PPh₃O, yield: 72.2%). LC/MS: 347.0 [M+H]⁺.

### Step 8: Preparation of 1-(4-((4-((2-fluoro-4-((1-(6-methylpyridin-3-yl)-1H-pyrazol-3-yl)oxy)phenyl)amino)-7-methoxyquinazolin-6-yl)amino)piperidin-1-yl)prop-2-en-1-one

To a solution of 2-fluoro-4-{[1-(6-methylpyridin-3-yl)pyrazo]-3-yl]oxy}aniline (1.24 g, 4.36 mmol) in MeCN (100 mL) stirred at room temperature was added 1-{4-[(4-chloro-7-methoxyquinazolin-6-yl)amino]piperidin-1-yl}prop-2-en-1-one (6.05 g, 25% purity, 4.36 mmol) and TsOH (225 mg, 1.31 mmol). The reaction mixture was stirred at 80° C. for 2 hours. The mixture was cooled to room temperature and some solid appeared. The solid was filtered and washed with MeCN. The resulting solid was dissolved with DCM and washed with saturated sodium bicarbonate solution. The organic phase was concentrated in vacuum to give a crude material which was purified by flash column chromatography (DCM/MeOH=10:1) to give the desired product (1.88 g, yield=72.5%), LC/MS: 595.0 [M+H]⁺; ¹H NMR (400 MHz, CD₃OD) δ=9.17 (s, 1H), 8.90 (d, J=2.8 Hz, 1H), 8.56 (d, J=2.8 Hz, 1H), 8.22 (s, 1H), 8.05 (dd, J=8.4, 2.8 Hz, 1H), 7.52 (t, J=8.8 Hz, 1H), 7.38 (d, J=8.4 Hz, 1H), 7.28-7.22 (m, 2H), 7.14-7.09 (m, 1H), 7.08 (s, 1H), 6.85 (dd, J=16.8, 10.4 Hz, 1H), 6.34 (d, J=2.8 Hz, 1H), 6.11 (dd, J=16.8, 2.4 Hz, 1H), 5.68 (dd, J=10.4, 2.4 Hz, 1H), 5.31 (d, J=8.4 Hz, 1H), 4.46 (d, J=12.4 Hz, 1H), 4.12 (d, J=12.4 Hz, 1H), 3.96 (s, 3H), 3.79-3.69 (m, 1H), 3.22 (t, J=12.4 Hz, 1H), 2.86 (t, J=12.0 Hz, 1H), 2.49 (s, 3H), 2.11-2.03 (m, 2H), 1.49-1.37 (m, 2H).

### Example 3: Preparation of 1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one (Compound 2)

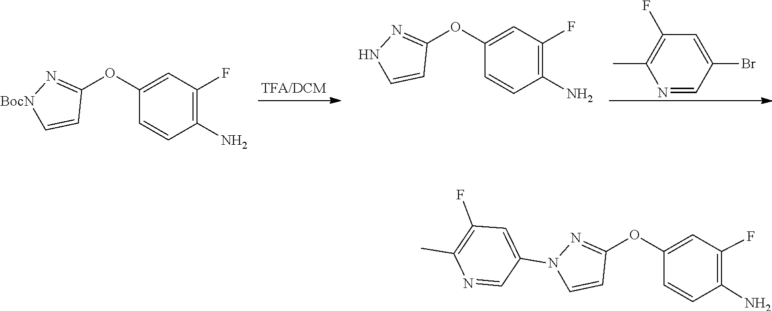

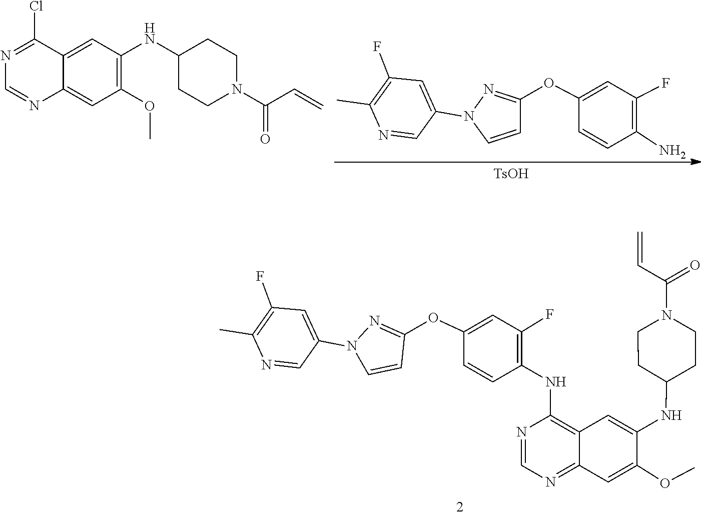

### Step 1: Preparation of 4-((1H-pyrazol-3-yl)oxy)-2-fluoroaniline

Tert-butyl 3-(4-amino-3-fluorophenoxy)-1H-pyrazole-1-carboxylate (3.28 g, 11.2 mmol, see preparation procedure in Example 8) was stirred in TFA/DCM (36 mL, 1:5) at room temperature for 2 hours. The mixture was concentrated in vacuum. The residue was dissolved in DCM (100 mL), washed with saturated sodium bicarbonate solution and brine. The organic layer was dried over Na₂SO₄ and concentrated in vacuum to give the desired product (1.73 g, yield=80.1%). LC/MS: 194.1 [M+H]⁺.

### Step 2: Preparation of 2-fluoro-4-((1-(5-fluoro-6-methylpyridin-3-yl)-1H-pyrazol-3-yl)oxy)aniline

To a solution of 2-fluoro-4-(1H-pyrazol-3-yloxy)aniline (2.5 g, 12.9 mol) in toluene (30 mL) stirred at room temperature was added 5-bromo-3-fluoro-2-methylpyridine (3.7 g, 19.4 mmol), (1R,2R)-1-N,2-N-dimethylcyclohexane-1,2-diamine (736 mg, 5.2 mol), K₂CO₃ (4.47 g, 32.4 mmol) and CuI (493 mg, 2.6 mmol). The reaction mixture was stirred at 100° C. under Ar for 12 hours. The mixture was diluted with EA (70 mL) and filtered through a Celite pad. The filtrate was concentrated in vacuum to give a crude product. The crude product was purified by flash column chromatography (PE/EA=1:1) to give the desired product (2 g, yield=51.1%), LC/MS: 303.1 [M+H]⁺; ¹H NMR (400 MHz, DMSO-d6) δ=8.76 (d, J=1.2 Hz, 1H), 8.50 (d, J=2.8 Hz, 1H), 8.03 (dd, J=10.8, 2.0 Hz, 1H), 6.98 (dd, J=12.0, 2.0 Hz, 1H), 6.82-6.76 (m, 2H), 6.09 (d, J=2.8 Hz, 1H), 5.04 (s, 2H), 2.45 (d, J=2.8 Hz, 3H).

### Step 3: Preparation of 1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one

To a solution of 1-{4-[(4-chloro-7-methoxyquinazolin-6-yl)amino]piperidin-1-yl}prop-2-en-1-one (5.74 g, 20% purity, 3.31 mmol, prepared in Example 2) in MeCN stirred at room temperature was added 2-fluoro-4-{[1-(5-fluoro-6-methylpyridin-3-yl)pyrazo]-3-yl]oxy}aniline (1 g, 3.31 mmol) and TsOH (172 mg, 1 mmol). The reaction mixture was stirred at 80° C. for 2 hours. The mixture was cooled to room temperature. The suspension mixture was filtered and washed with MeCN. The resulting solid was dissolved with DCM and the solution was washed with saturated sodium bicarbonate. The organic phase was dried and concentrated to give a crude material which was purified by flash column chromatography (MeOH/DCM=1:10) to give the desired product (1.08 g, yield=53.3%), LC/MS: 613.0 [M+H]⁺; ¹H NMR (400 MHz, DMSO-d6) δ=9.17 (s, 1H), 8.81 (d, J=1.6 Hz, 1H), 8.63 (d, J=2.8 Hz, 1H), 8.21 (s, 1H), 8.10 (dd, J=10.8, 2.4 Hz, 1H), 7.53 (t, J=8.8 Hz, 1H), 7.30-7.22 (m, 2H), 7.16-7.10 (m, 1H), 7.07 (s, 1H), 6.86 (dd, J=16.8, 10.8 Hz, 1H), 6.39 (d, J=2.4 Hz, 1H), 6.11 (dd, J=16.8, 2.4, 1H), 5.68 (dd, J=10.4, 2.4 Hz, 1H), 5.32 (d, J=8.4 Hz, 1H), 4.46 (d, J=13.2, 1H), 4.13 (d, J=10.8 Hz, 1H), 3.96 (s, 3H), 3.80-3.70 (m, 1H), 3.23 (t, J=12.4 Hz, 1H), 2.86 (t, J=12.0 Hz, 1H), 2.47 (d, J=2.8 Hz, 3H), 2.07 (s, 2H), 1.50-1.38 (m, 2H).

### Example 4: Preparation of 1-(4-((4-((2-fluoro-4-((4-(5-isopropyl-1,3,4-oxadiazol-2-yl)thiazol-2-yl)oxy)phenyl)amino)-7-methoxyquinazolin-6-yl)amino)piperidin-1-yl)prop-2-en-1-one (Compound 4)

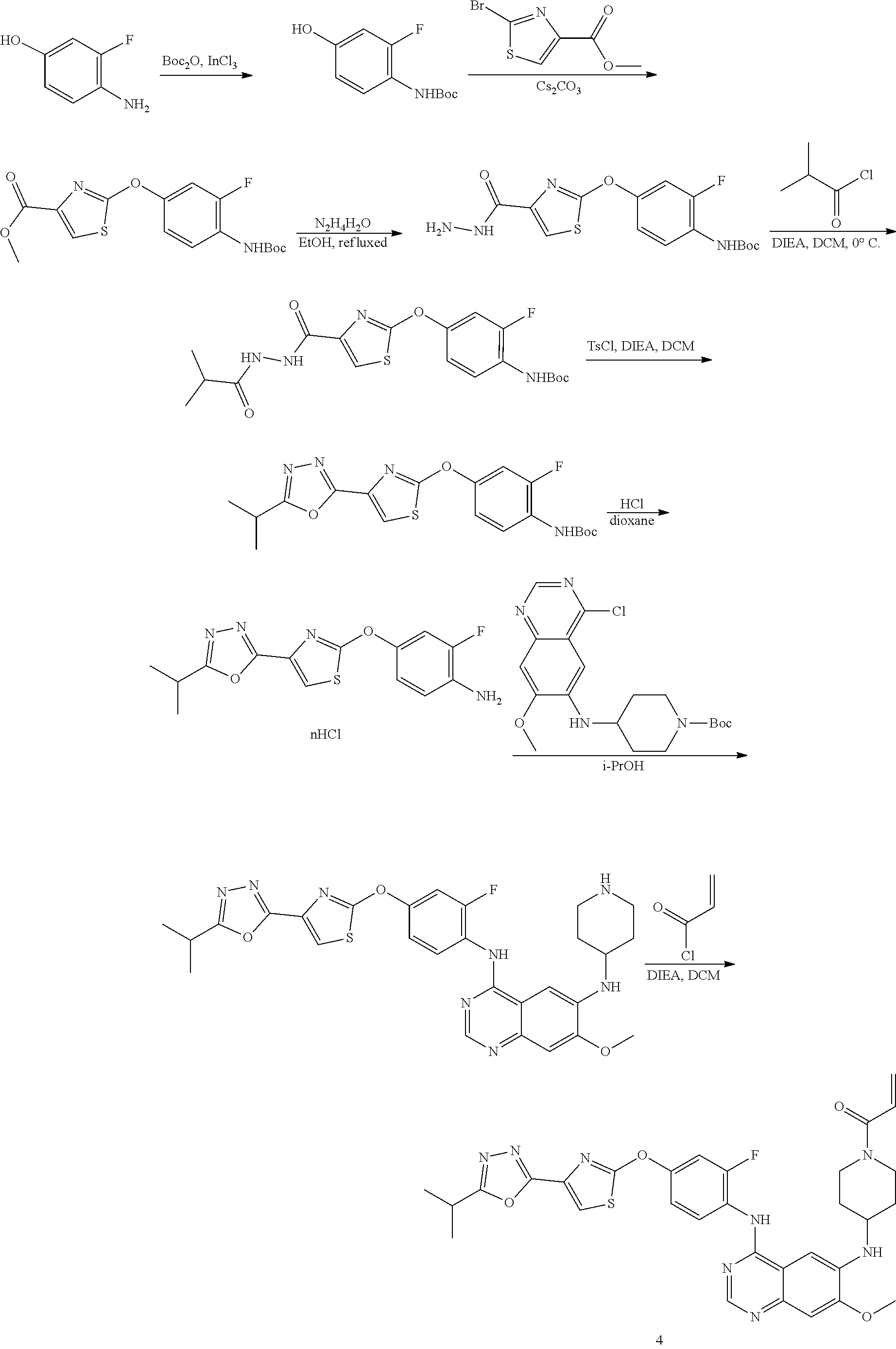

### Step 1: Preparation of tert-butyl (2-fluoro-4-hydroxyphenyl)carbamate

A solution of 4-amino-3-fluorophenol (24 g, 190 mmol) and InCl₃ (62 g, 285 mmol) in Boc₂O (100 mL) was stirred at 25° C. for 2 h. The reaction solution was concentrated under vacuum and the crude product was purified by flash column chromatography (PE/EA=4:1) to get the product tert-butyl (2-fluoro-4-hydroxyphenyl)carbamate (39 g, 90%). LC/MS: 228 [M+H]⁺.

### Step 2: Preparation of methyl 2-(4-((tert-butoxycarbonyl)amino)-3-fluorophenoxy)thiazole-4-carboxylate

A solution of tert-butyl (2-fluoro-4-hydroxyphenyl)carbamate (3.0 g, 13.2 mmol), methyl 2-bromothiazole-4-carboxylate (2.9 g, 13.2 mmol) and Cs₂CO₃ (8.6 g, 26.4 mmol) in THE (40 mL) was stirred at 60° C. for 5 h. The reaction solution was concentrated under vacuum. The residue was diluted with water (100 mL) and extracted by EA (50 mL×3). The combined organic layers were washed by brine and dried over Na₂SO₄. After filtration, the filtrate was concentrated under pressure and the crude product was purified by flash column chromatography (PE/EA=3:1) to get the product methyl 2-(4-((tert-butoxycarbonyl)amino)-3-fluorophenoxy)thiazole-4-carboxylate (4.0 g, 66%). LC/MS: 369 [M+H]⁺.

### Step 3: Preparation of tert-butyl (2-fluoro-4-((4-(hydrazinecarbonyl)thiazol-2-yl)oxy)phenyl)carbamate

A solution of methyl 2-(4-((tert-butoxycarbonyl)amino)-3-fluorophenoxy)thiazole-4-carboxylate (3.8 g, 10.3 mmol) and N₂H₄·H₂O (1 g, 20.0 mmol) in EtOH (50 mL) was stirred at 80° C. for 16 h. The reaction solution was concentrated under vacuum. The residue was diluted with water (100 mL) and extracted by EA (50 mL×3). The combined organic layers were washed with brine and dried over Na₂SO₄. After filtration, the filtrate was concentrated under reduced pressure and the crude product was purified by flash column chromatography (PE/EA=1:1) to get the product tert-butyl (2-fluoro-4-((4-(hydrazinecarbonyl)thiazol-2-yl)oxy)phenyl)carbamate (3.0 g, 67%). LC/MS: 369 [M+H]⁺.

### Step 4: Preparation of tert-butyl (2-fluoro-4-((4-(2-isobutyrylhydrazine-1-carbonyl)thiazol-2-yl)oxy)phenyl)carbamate

A solution of tert-butyl (2-fluoro-4-((4-(hydrazinecarbonyl)thiazol-2-yl)oxy)phenyl)carbamate (2.0 g, 5.4 mmol), 2-methylpropanoyl chloride (1.2 g, 10.9 mmol) and DIEA (1.4 g, 10.9 mmol) in DCM (20 mL) was stirred at 0° C. for 2 h. The reaction solution was washed with saturated sodium bicarbonate solution and the organic layer was dried over Na₂SO₄. After filtration, the filtrate was concentrated under vacuum to give the crude product tert-butyl (2-fluoro-4-((4-(2-isobutyrylhydrazine-1-carbonyl)thiazol-2-yl)oxy)phenyl)carbamate (2.5 g, crude) as a yellow solid. LC/MS: 439 [M+H]⁺.

### Step 5: Preparation of tert-butyl (2-fluoro-4-((4-(5-isopropyl-1,3,4-oxadiazol-2-yl)thiazol-2-yl)oxy)phenyl)carbamate

A solution of tert-butyl (2-fluoro-4-((4-(2-isobutyrylhydrazine-1-carbonyl)thiazol-2-yl)oxy)phenyl)carbamate (2.5 g, 2.7 mmol), 4-methylbenzenesulfonyl chloride (1.6 g, 8.6 mmol) and DIEA (1.5 g, 11.4 mmol) in DCM (30 mL) was stirred at 25° C. for 16 h. The reaction solution was washed with brine and the organic layer was dried over Na₂SO₄. After filtration, the filtrate was concentrated under vacuum and the crude product was purified by flash column chromatography (PE/EA=4:1) to give the product tert-butyl (2-fluoro-4-((4-(5-isopropyl-1,3,4-oxadiazol-2-yl)thiazol-2-yl)oxy)phenyl) carbamate (900 mg, 30%) as a yellow oil. LC/MS: 421 [M+H]⁺.

### Step 6: Preparation of 2-fluoro-4-((4-(5-isopropyl-1,3,4-oxadiazol-2-yl)thiazol-2-yl)oxy)aniline

A solution of tert-butyl (2-fluoro-4-((4-(5-isopropyl-1,3,4-oxadiazol-2-yl)thiazol-2-yl)oxy)phenyl)carbamate (900 mg) in HCl/dioxane (10 mL, 4 N) was stirred at 25° C. for 2 h. The reaction solution was concentrated under vacuum and the crude product (900 mg) was used in the next step without further purification. LC/MS: 321 [M+H]⁺.

### Step 7: Preparation of N4-(2-fluoro-4-((4-(5-isopropyl-1,3,4-oxadiazol-2-yl)thiazol-2-yl)oxy)phenyl)-7-methoxy-N6-(piperidin-4-yl)quinazoline-4,6-diamine

A solution of 2-fluoro-4-((4-(5-isopropyl-1,3,4-oxadiazol-2-yl)thiazol-2-yl)oxy)aniline HCl salt (300 mg, 0.94 mmol) and tert-butyl 4-((4-chloro-7-methoxyquinazolin-6-yl)amino)piperidine-1-carboxylate (370 mg, 0.94 mmol) in i-PrOH (10 mL) was stirred at 25° C. for 16 hr. LC-MS analysis indicated the Boc group was cleaved during the reaction. The reaction solution was concentrated under vacuum to give a crude product of N4-(2-fluoro-4-((4-(5-isopropyl-1,3,4-oxadiazol-2-yl)thiazol-2-yl)oxy)phenyl)-7-methoxy-N6-(piperidin-4-yl)quinazoline-4,6-diamine. LC/MS: 577 [M+H]⁺.

### Step 8: Preparation of 1-(4-((4-((2-fluoro-4-((4-(5-isopropyl-1,3,4-oxadiazol-2-yl)thiazol-2-yl)oxy)phenyl)amino)-7-methoxyquinazolin-6-yl)amino)piperidin-1-yl)prop-2-en-1-one

To a solution of N4-(2-fluoro-4-((4-(5-isopropyl-1,3,4-oxadiazol-2-yl)thiazol-2-yl)oxy)phenyl)-7-methoxy-N6-(piperidin-4-yl)quinazoline-4,6-diamine (80 mg, 0.14 mmol) and DIEA (36 mg, 0.28 mmol) in dry DCM (5 mL) was added acryloyl chloride (13 mg, 0.14 mmol) at −78° C. and the mixture was stirred at −78° C. for 10 min. The reaction solution was quenched by H₂O, concentrated under vacuum and purified via prep-HPLC [MeCN—H₂O (0.1% TFA), 30%-55%] to give the product 1-(4-((4-((2-fluoro-4-((4-(5-isopropyl-1,3,4-oxadiazol-2-yl)thiazol-2-yl)oxy)phenyl)amino)-7-methoxyquinazolin-6-yl)amino)piperidin-1-yl)prop-2-en-1-one (20 mg, 22%) as a yellow solid. LC/MS: 631 [M+H]⁺. ¹H NMR (400 MHz, CD₃OD) δ8.55 (s, 1H), 7.99 (s, 1H), 7.69 (t, J=8.6 Hz, 1H), 7.53 (dd, J=10.6, 2.5 Hz, 1H), 7.45 (s, 1H), 7.43-7.37 (m, 1H), 7.19-7.11 (m, 1H), 6.82 (dd, J=16.8, 10.7 Hz, 1H), 6.22 (dd, J=16.8, 1.9 Hz, 1H), 5.76 (dd, J=10.6, 1.9 Hz, 1H), 4.68-4.56 (m, 1H), 4.27-4.16 (m, 1H), 4.12 (s, 3H), 3.90-3.80 (m, 1H), 3.40-3.32 (m, 1H), 3.29-3.25 (m, 1H), 3.05-2.92 (m, 1H), 2.27-2.14 (m, 2H), 1.62-1.51 (m, 2H), 1.43 (d, J=7.0 Hz, 6H).

### Example 5: Preparation of 1-(4-((4-((2-fluoro-4-((5-(6-methylpyridin-3-yl)thiazol-2-yl)oxy)phenyl)amino)-7-methoxyquinazolin-6-yl)amino)piperidin-1-yl)prop-2-en-1-one (Compound 5)

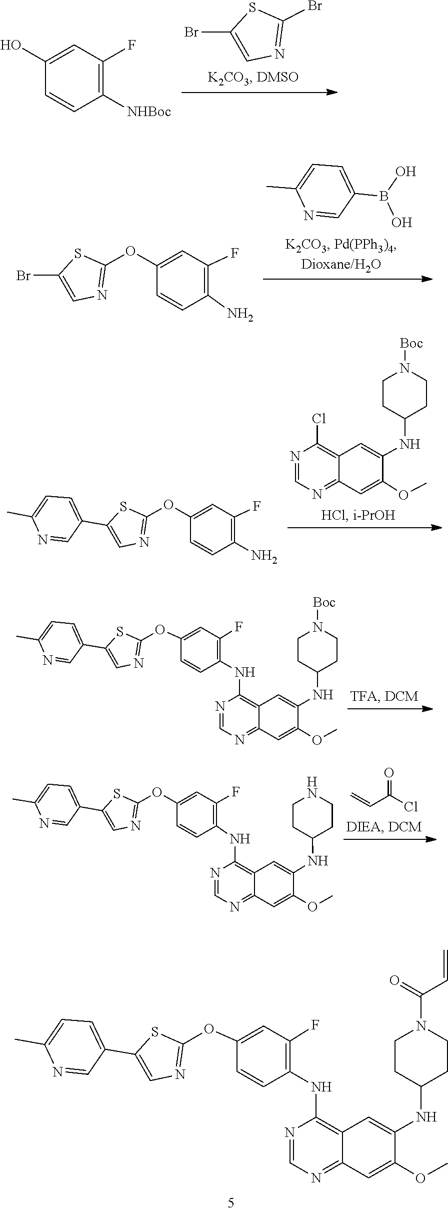

### Step 1: Preparation of 4-((5-bromothiazol-2-yl)oxy)-2-fluoroaniline

To a solution of tert-butyl (2-fluoro-4-hydroxyphenyl)carbamate (1.2 g, 5.29 mmol) in DMSO (30 mL) was added 2,5-dibromothiazole (1.28 g, 5.29 mmol) and K₂CO₃ (1.1 g, 7.94 mmol). The reaction mixture was stirred for 2 hours at 130° C. LC-MS analysis indicated the Boc group was partially cleaved during the reaction. The reaction solution was poured into EA (100 mL). The organic layer was washed with water (60 mL×3), dried over Na₂SO₄, concentrated in vacuo and purified with Combi-Flash (EA/PE=1:3) to afford 4-((5-bromothiazol-2-yl)oxy)-2-fluoroaniline (880 mg, 58%) as a white solid. LC/MS: 289 [M+H]⁺.

### Step 2: Preparation of 2-fluoro-4-((5-(6-methylpyridin-3-yl)thiazol-2-yl)oxy)aniline

To a solution of 4-((5-bromothiazol-2-yl)oxy)-2-fluoroaniline (800 mg, 2.77 mmol) in dioxane/H₂O (32 mL/8 mL) was added (6-methylpyridin-3-yl)boronic acid (380 mg, 2.77 mmol), Pd(PPh₃)₄ (320 mg, 0.277 mmol) and K₂CO₃ (756 mg, 5.54 mmol). The mixture was stirred for 6 hours at 90° C. under N₂ atmosphere. The reaction solution was concentrated in vacuo and purified by Combi-Flash (DCM/MeOH=11:1) to give 2-fluoro-4-((5-(6-methylpyridin-3-yl)thiazol-2-yl)oxy)aniline (730 mg, 87%) as a light yellow solid. LC/MS: 302 [M+H]⁺.

### Step 3: Preparation of tert-butyl 4-((4-((2-fluoro-4-((5-(6-methylpyridin-3-yl)thiazol-2-yl)oxy)phenyl)amino)-7-methoxyquinazolin-6-yl)amino)piperidine-1-carboxylate

To a solution of 2-fluoro-4-((5-(6-methylpyridin-3-yl)thiazol-2-yl)oxy)aniline (110 mg, 0.37 mmol) in i-PrOH (15 mL) was added tert-butyl 4-((4-chloro-7-methoxyquinazolin-6-yl)amino)piperidine-1-carboxylate (145 mg, 0.37 mmol) and HCl/dioxane (4N, 0.1 mL). The reaction mixture was stirred for 16 hours at room temperature. The solution was concentrated and purified by Combi-Flash (DCM/MeOH=11/1) to give tert-butyl 4-((4-((2-fluoro-4-((5-(6-methylpyridin-3-yl)thiazol-2-yl)oxy)phenyl)amino)-7-methoxyquinazolin-6-yl)amino)piperidine-1-carboxylate (160 mg, 67%) as a light yellow solid. LC/MS: 658 [M+H]⁺.

### Step 4: Preparation of N4-(2-fluoro-4-((5-(6-methylpyridin-3-yl)thiazol-2-yl)oxy)phenyl)-7-methoxy-N6-(piperidin-4-yl)quinazoline-4,6-diamine

A solution of tert-butyl 4-((4-((2-fluoro-4-((5-(6-methylpyridin-3-yl)thiazol-2-yl)oxy)phenyl)amino)-7-methoxyquinazolin-6-yl)amino)piperidine-1-carboxylate (150 mg, 0.23 mmol) in DCM/TFA (6 mL/2 mL) was stirred for 1 hour at room temperature. The reaction mixture was poured into water (60 mL) and extracted with DCM (50 mL×3). The organic layer was combined, dried over Na₂SO₄ and concentrated in vacuo to provide N4-(2-fluoro-4-((5-(6-methylpyridin-3-yl)thiazol-2-yl)oxy)phenyl)-7-methoxy-N6-(piperidin-4-yl)quinazoline-4,6-diamine (120 mg, crude) as an off-white solid. LC/MS: 558 [M+H]⁺.

### Step 5: Preparation of 1-(4-((4-((2-fluoro-4-((5-(6-methylpyridin-3-yl)thiazol-2-yl)oxy)phenyl)amino)-7-methoxyquinazolin-6-yl)amino)piperidin-1-yl)prop-2-en-1-one

To a solution of N4-(2-fluoro-4-((5-(6-methylpyridin-3-yl)thiazol-2-yl)oxy)phenyl)-7-methoxy-N6-(piperidin-4-yl)quinazoline-4,6-diamine (110 mg, 0.2 mmol) in CH2Cl2 (10 mL) was added DIEA (52 mg, 0.4 mmol) and acryloyl chloride (18 mg, 0.2 mmol) at −78° C. The solution was stirred for 3 min at −78° C. The reaction solution was concentrated and purified by Prep-HPLC [ACN—H2O (0.1% TFA) 40%-70%]. The desired fractions were pooled and lyophilized to give 1-(4-((4-((2-fluoro-4-((5-(6-methylpyridin-3-yl)thiazol-2-yl)oxy)phenyl)amino)-7-methoxyquinazolin-6-yl)amino)piperidin-1-yl)prop-2-en-1-one (14.8 mg, 12%) as a white solid. LC/MS: 612 [M+H]⁺; ¹H NMR (400 MHz, CD₃OD) δ8.83 (s, 1H), 8.54 (s, 1H), 8.29 (d, J=8.4 Hz, 1H), 7.80 (s, 1H), 7.73-7.63 (m, 2H), 7.48 (m, 1H), 7.44 (s, 1H), 7.39-7.33 (m, 1H), 7.15 (s, 1H), 6.82-6.80 (m, 1H), 6.22-6.21 (m, 1H), 5.76-5.73 (m, 1H), 4.61-4.60 (m, 1H), 4.21-4.19 (m, 1H), 4.12 (s, 3H), 3.90-3.80 (m, 1H), 3.36-3.33 (m, 1H), 3.05-2.97 (m, 1H), 2.68 (s, 3H), 2.21-2.19 (m, 2H), 1.66-1.49 (m, 2H).

### Example 6: Preparation of 1-(4-((4-((2-fluoro-4-((4-(6-methylpyridin-3-yl)thiazol-2-yl)oxy)phenyl)amino)-7-methoxyquinazolin-6-yl)amino)piperidin-1-yl)prop-2-en-1-one (Compound 6)

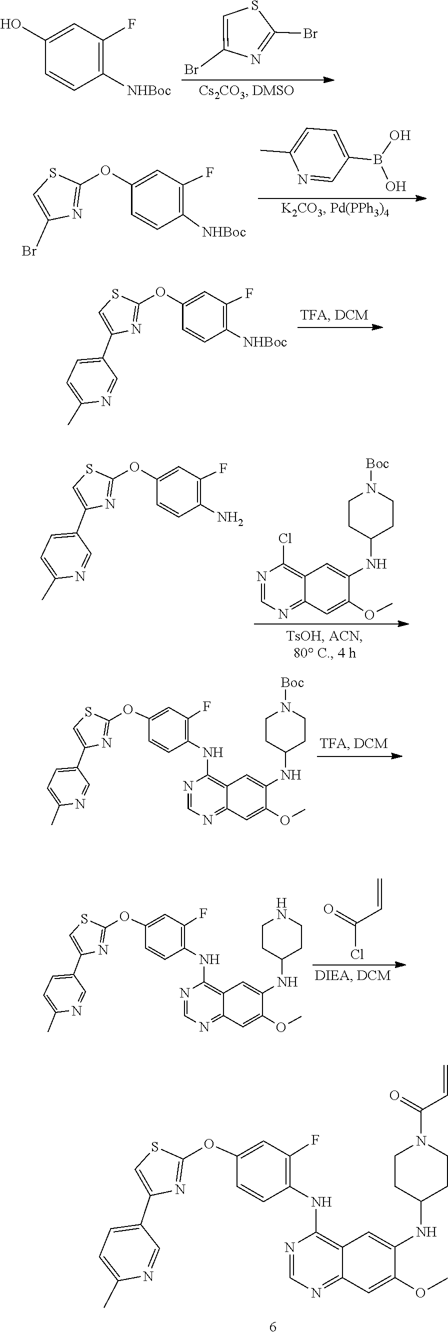

### Step 1: Preparation of tert-butyl (4-((4-bromothiazol-2-yl)oxy)-2-fluorophenyl)carbamate

A solution of tert-butyl N-(2-fluoro-4-hydroxyphenyl)carbamate (1.0 g, 4.4 mmol), 2,4-dibromo-1,3-thiazole (1.1 g, 4.4 mmol) and Cs₂CO₃ (2.9 g, 8.8 mmol) in THE (20 mL) was stirred at 60° C. for 5 hr. The reaction solution was concentrated under vacuum. The residue was diluted with water (100 mL) and extracted by EtOAc (50 mL×3). The combined organic layers were washed by brine and dried over Na₂SO₄. After filtration, the filtrate was concentrated under reduced pressure and the crude product was purified by flash chromatography (PE/EA=4:1) to give tert-butyl (4-((4-bromothiazol-2-yl)oxy)-2-fluorophenyl)carbamate (1.0 g, 53%). LC/MS: 389 [M+H]⁺.

### Step 2: Preparation of tert-butyl (2-fluoro-4-((4-(6-methylpyridin-3-yl)thiazol-2-yl)oxy)phenyl)carbamate

A solution of tert-butyl (4-((4-bromothiazol-2-yl)oxy)-2-fluorophenyl)carbamate (1.0 g, 2.57 mmol), (6-methylpyridin-3-yl)boronic acid (352 mg, 2.57 mmol), Pd(PPh₃)₄ (300 mg, 0.26 mmol) and K₂CO₃ (710 mg, 5.14 mmol) in dioxane (10 mL) and H₂O (2.5 mL) was stirred at 90° C. for 5 hr under N₂. The reaction solution was concentrated under vacuum. The residue was diluted with water (100 mL) and extracted with EtOAc (50 mL×3). The combined organic layers were wash with brine and dried over Na₂SO₄. After filtration, the filtrate was concentrated under reduced pressure and the crude product was purified by flash chromatography (PE/EA=2:1) to give tert-butyl (2-fluoro-4-((4-(6-methylpyridin-3-yl)thiazol-2-yl)oxy)phenyl)carbamate (710 mg, 62%). LC/MS: 402 [M+H]⁺.

### Step 3: Preparation of 2-fluoro-4-((4-(6-methylpyridin-3-yl)thiazol-2-yl)oxy)aniline

A solution of tert-butyl (2-fluoro-4-((4-(6-methylpyridin-3-yl)thiazol-2-yl)oxy)phenyl)carbamate (700 mg, 1.7 mmol) and TFA (2 mL) in DCM (8 mL) was stirred at 25° C. for 2 hr. The reaction solution was concentrated under vacuum to give the crude product 2-fluoro-4-((4-(6-methylpyridin-3-yl)thiazol-2-yl)oxy)aniline (1 g, crude) as a yellow solid. LC/MS: 302 [M+H]⁺.

### Step 4: Preparation of tert-butyl 4-((4-((2-fluoro-4-((4-(6-methylpyridin-3-yl)thiazol-2-yl)oxy)phenyl)amino)-7-methoxyquinazolin-6-yl)amino)piperidine-1-carboxylate

A solution of 2-fluoro-4-((4-(6-methylpyridin-3-yl)thiazol-2-yl)oxy)aniline (200 mg, 0.66 mmol), tert-butyl 4-((4-chloro-7-methoxyquinazolin-6-yl)amino)piperidine-1-carboxylate (390 mg, 0.99 mmol) and TsOH (57 mg, 0.33 mmol) in ACN (5 mL) was stirred at 80° C. for 4 hr. The reaction solution was concentrated under vacuum and the crude product was purified by flash column chromatography (DCM/MeOH=10:1) to give tert-butyl 4-((4-((2-fluoro-4-((4-(6-methylpyridin-3-yl)thiazol-2-yl)oxy)phenyl)amino)-7-methoxyquinazolin-6-yl)amino)piperidine-1-carboxylate (100 mg, 21%) as a yellow solid. LC/MS: 658 [M+H]⁺.

### Step 5: Preparation of N4-(2-fluoro-4-((4-(6-methylpyridin-3-yl)thiazol-2-yl)oxy)phenyl)-7-methoxy-N6-(piperidin-4-yl)quinazoline-4,6-diamine

A solution of tert-butyl 4-((4-((2-fluoro-4-((4-(6-methylpyridin-3-yl)thiazol-2-yl)oxy)phenyl)amino)-7-methoxyquinazolin-6-yl)amino)piperidine-1-carboxylate (100 mg, 0.15 mmol) and TFA (2 mL) in DCM (8 mL) was stirred at 25° C. for 2 hr. The reaction solution was concentrated under vacuum. The residue was dissolved with EtOAc, washed with saturated sodium bicarbonate solution and dried over Na₂SO₄. After filtration, the filtrate was concentrated under vacuum to give the crude product N4-(2-fluoro-4-((4-(6-methylpyridin-3-yl)thiazol-2-yl)oxy)phenyl)-7-methoxy-N6-(piperidin-4-yl)quinazoline-4,6-diamine (70 mg, 80%) as a yellow solid. LC/MS: 558 [M+H]⁺.

### Step 6: Preparation of 1-(4-((4-((2-fluoro-4-((4-(6-methylpyridin-3-yl)thiazol-2-yl)oxy)phenyl)amino)-7-methoxyquinazolin-6-yl)amino)piperidin-1-yl)prop-2-en-1-one

To a solution of N4-(2-fluoro-4-((4-(6-methylpyridin-3-yl)thiazol-2-yl)oxy)phenyl)-7-methoxy-N6-(piperidin-4-yl)quinazoline-4,6-diamine (60 mg, 0.1 mmol) and DIEA (28 mg, 0.2 mmol) in DCM (2 mL) was added acryloyl chloride (10 mg, 0.1 mmol) at −78° C. and the solution was stirred at −78° C. for 10 min. The reaction was quenched with H2O, concentrated under vacuum and purified via prep-HPLC [ACN—H2O (0.1% TFA), 30%-55%] to give 1-(4-((4-((2-fluoro-4-((4-(6-methylpyridin-3-yl)thiazol-2-yl)oxy)phenyl)amino)-7-methoxyquinazolin-6-yl)amino)piperidin-1-yl)prop-2-en-1-one (15 mg, 21%) as a yellow solid. LC/MS: 612 [M+H]⁺; ¹H NMR (400 MHz, CD₃OD) δ9.05 (s, 1H), 8.82-8.72 (m, 1H), 8.55 (s, 1H), 7.92-7.81 (m, 2H), 7.70 (t, J=8.6 Hz, 1H), 7.51 (dd, J=10.6, 2.5 Hz, 1H), 7.45 (s, 1H), 7.43-7.36 (m, 1H), 7.20-7.14 (m, 1H), 6.82 (dd, J=16.8, 10.7 Hz, 1H), 6.22 (dd, J=16.8, 1.9 Hz, 1H), 5.76 (dd, J=10.7, 2.0 Hz, 1H), 4.65-4.55 (m, 1H), 4.25-4.18 (m, 1H), 4.16 (s, 3H), 3.91-3.80 (m, 1H), 3.41-3.33 (m, 1H), 3.03-2.94 (m, 1H), 2.77 (s, 3H), 2.26-2.16 (m, 2H), 1.67-1.47 (m, 2H).

### Example 7: Preparation of 1-(4-((4-((4-([1,2,4]triazolo[1,5-a]pyridin-7-yloxy)-3-chlorophenyl)amino)-7-methoxyquinazolin-6-yl)amino)piperidin-1-yl)prop-2-en-1-one (Compound 7)

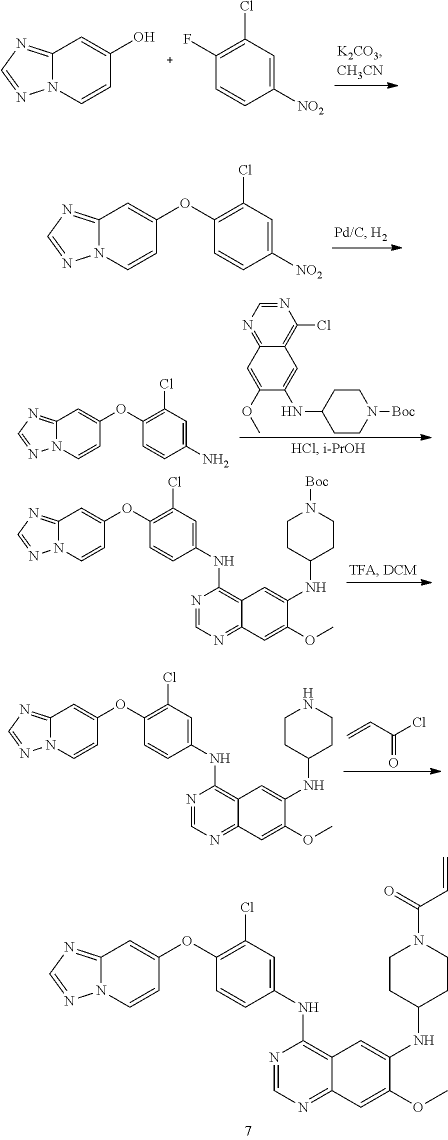

### Step 1: Preparation of 7-(2-chloro-4-nitrophenoxy)-[1,2,4]triazolo[1,5-a]pyridine

To a solution of [1,2,4]triazolo[1,5-a]pyridin-7-ol (300 mg, 2.22 mmol), 2-chloro-1-fluoro-4-nitrobenzene (388 mg, 2.22 mmol) in CH₃CN (20 mL) was added K₂CO₃ (304 mg, 2.22 mmol). The reaction mixture was stirred at 80° C. for 2 hours. The reaction solution was poured into EtOAc (100 mL). The organic layer was washed with water (60 mL×3), dried over Na₂SO₄ and concentrated in vacuo. The crude product was purified by Combi-Flash (EA/PE=1:3) to give 7-(2-chloro-4-nitrophenoxy)-[1,2,4]triazolo[1,5-a]pyridine (500 mg, 77.6%) as a white solid. LC/MS: 291 [M+H]⁺.

### Step 2: Preparation of 4-([1,2,4]triazolo[1,5-a]pyridin-7-yloxy)-3-chloroaniline

To a solution of 7-(2-chloro-4-nitrophenoxy)-[1,2,4]triazolo[1,5-a]pyridine (480 mg, 2.77 mmol) in MeOH (20 mL) was added Pd/C (500 mg). The solution was stirred at 25° C. for 3 hr under H₂ atmosphere. The reaction solution was concentrated and purified by Combi-Flash (DCM/MeOH=11:1) to give 4-([1,2,4]triazolo[1,5-a]pyridin-7-yloxy)-3-chloroaniline (378 mg, 88%) as a light yellow solid. LC/MS: 261 [M+H]⁺.

### Step 3: Preparation of tert-butyl 4-((4-((4-([1,2,4]triazolo[1,5-a]pyridin-7-yloxy)-3-chlorophenyl)amino)-7-methoxyquinazolin-6-yl)amino)piperidine-1-carboxylate

To a solution of 7-(2-chloro-4-nitrophenoxy)-[1,2,4]triazolo[1,5-a]pyridine (96 mg, 0.37 mmol), tert-butyl 4-((4-chloro-7-methoxyquinazolin-6-yl)amino)piperidine-1-carboxylate (145 mg, 0.37 mmol) in i-PrOH (15 mL) was added HCl/dioxane (4N, 0.1 mL). The reaction mixture was stirred at room temperature for 16 hours. The solution was concentrated and purified by Combi-Flash (DCM/MeOH=11/1) to give tert-butyl 4-((4-((4-([1,2,4]triazolo[1,5-a]pyridin-7-yloxy)-3-chlorophenyl)amino)-7-methoxyquinazolin-6-yl)amino)piperidine-1-carboxylate (141 mg, 61.8%) as a light yellow solid. LC/MS: 617 [M+H]⁺.

### Step 4: Preparation of N4-(4-([1,2,4]triazolo[1,5-a]pyridin-7-yloxy)-3-chlorophenyl)-7-methoxy-N6-(piperidin-4-yl)quinazoline-4,6-diamine

A solution of tert-butyl 4-((4-((4-([1,2,4]triazolo[1,5-a]pyridin-7-yloxy)-3-chlorophenyl)amino)-7-methoxyquinazolin-6-yl)amino)piperidine-1-carboxylate (141 mg, 0.23 mmol) in DCM/TFA (6 mL/2 mL) was stirred at room temperature for 1 hour. The reaction mixture was poured into water (60 mL) and extracted with DCM (50 mL×3). The organic layer was combined, dried over Na₂SO₄ and concentrated in vacuo to afford N4-(4-([1,2,4]triazolo[1,5-a]pyridin-7-yloxy)-3-chlorophenyl)-7-methoxy-N6-(piperidin-4-yl)quinazoline-4,6-diamine (103 mg) as an off-white solid. LC/MS: 518 [M+H]⁺.

### Step 5: Preparation of 1-(4-((4-((4-([1,2,4]triazolo[1,5-a]pyridin-7-yloxy)-3-chlorophenyl)amino)-7-methoxyquinazolin-6-yl)amino)piperidin-1-yl)prop-2-en-1-one (7)

To a solution of N4-(4-([1,2,4]triazolo[1,5-a]pyridin-7-yloxy)-3-chlorophenyl)-7-methoxy-N6-(piperidin-4-yl)quinazoline-4,6-diamine (103 mg, 0.2 mmol) in DCM (10 mL) was added DIEA (52 mg, 0.4 mmol) and acryloyl chloride (18 mg, 0.2 mmol) at −78° C. The solution was stirred for 3 min at −78° C. The reaction solution was concentrated and purified by Prep-HPLC, using a gradient of 0.1% TFA/ACN from 70% to 40%, and desired fractions were pooled and lyophilized to give the titled compound (14.8 mg, 13%) as a white solid. LC/MS: 571 [M+H]⁺; ¹H NMR (400 MHz, CD₃OD) δ8.82-8.80 (m, 2H), 8.65 (s, 1H), 8.05-8.03 (m, 1H), 7.77-7.75 (m, 1H), 7.51-7.49 (m, 1H), 7.34 (s, 1H), 7.14 (s, 1H), 6.83-6.82 (m, 2H), 6.20 (d, J=16.8, 2.0 Hz 1H), 5.78-5.76 (m, 1H), 4.59-4.57 (m, 1H), 4.12 (s, 3H), 3.92-3.90 (m, 1H), 3.34-3.33 (m, 1H), 3.01-3.00 (m, 3H), 2.22-2.20 (m, 2H), 1.57-1.55 (m, 2H).

### Example 8: (S)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one (Compound 8)

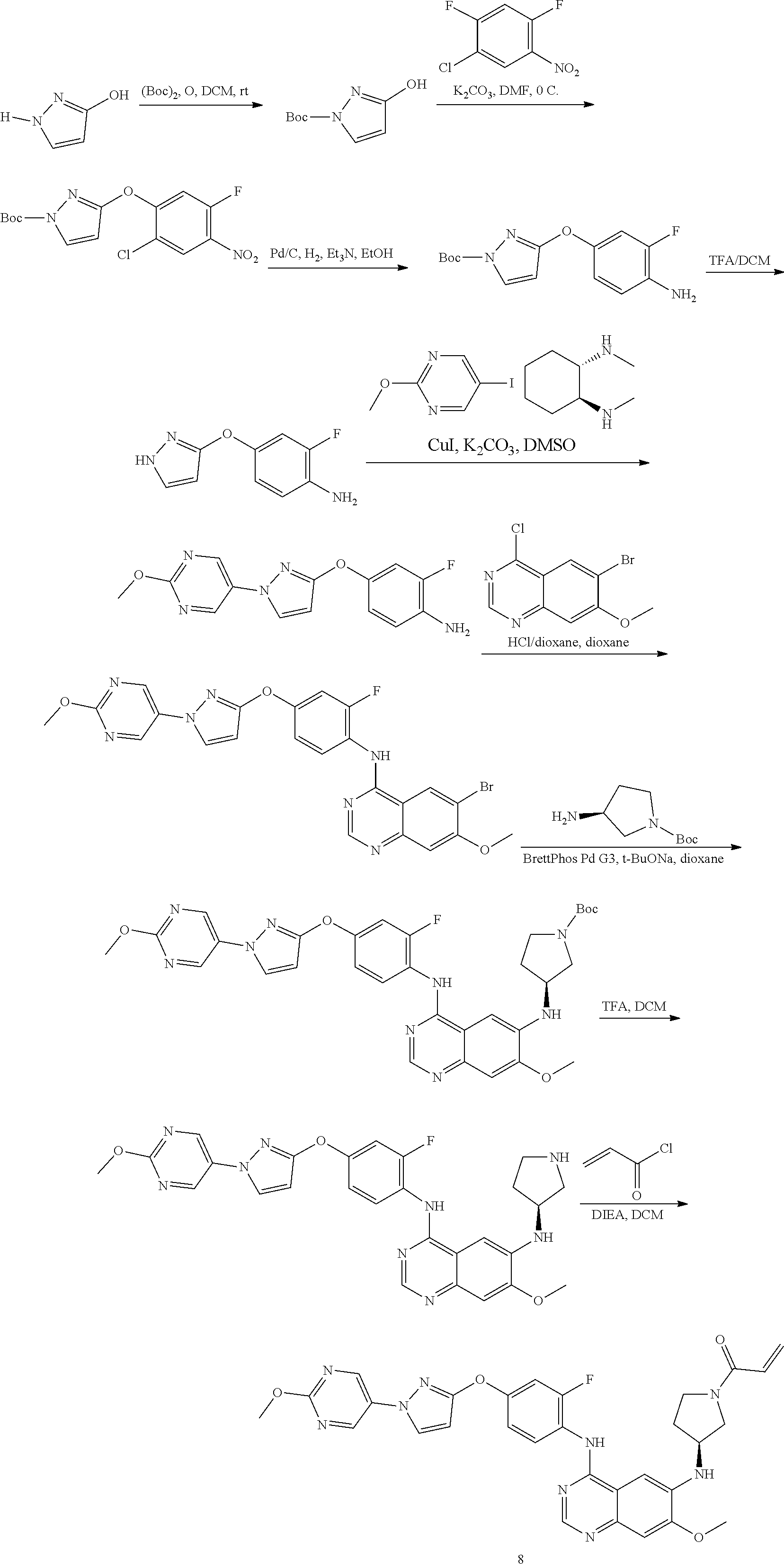

### Step 1: Preparation of tert-butyl 3-hydroxy-1H-pyrazole-1-carboxylate

To a solution of 1H-pyrazol-3-ol (100 g, 1.189 mol) and Et3N (132 g, 1.309 mol) in DCM (2 L) was added (Boc)₂O (286 g, 1.308 mol). The mixture was stirred at 25° C. for 16 h.

The solution was concentrated in vacuo and the residue was purified by silica gel column chromatography (PE:EA=100:20) to give tert-butyl 3-hydroxy-1H-pyrazole-1-carboxylate (165 g, 80%) as a white solid. LC-MS: 369.1 [2M+H]⁺.

### Step 2: Preparation of tert-butyl 3-(2-chloro-5-fluoro-4-nitrophenoxy)-1H-pyrazole-1-carboxylate

To a solution of tert-butyl 3-hydroxy-1H-pyrazole-1-carboxylate (110 g, 0.594 mol) and 1-chloro-2,4-difluoro-5-nitrobenzene (115 g, 0.594 mol) in DMF (1.1 L) was added K₂CO₃ (164.2 g, 1.188 mol) at 0° C. the mixture was stirred at 0° C. for 2 h. The mixture was diluted with water (2 L), extracted with EA (3×2000 mL). The organic phase was washed with water (3 L), dried over sodium sulphate and evaporated in vacuo to give a crude product. The crude material was purified by silica gel column chromatography (PE:EA=40/1) to provide tert-butyl 3-(2-chloro-5-fluoro-4-nitrophenoxy)-1H-pyrazole-1-carboxylate (121 g, 51%) as a white solid. LC-MS: 380.0 [M+Na]⁺.

### Step 3: Preparation of tert-butyl 3-(4-amino-3-fluorophenoxy)-1H-pyrazole-1-carboxylate

A mixture of tert-butyl 3-(2-chloro-5-fluoro-4-nitrophenoxy)-1H-pyrazole-1-carboxylate (70 g, 0.195 mol), Pd/C (35 g) and Et₃N (98.7 g, 0.975 mol) in EtOH (200 mL) was stirred at 50° C. for 9.5 h under H₂ atmosphere. Then the mixture was stirred at 25° C. for another 12 h. The solution was filtered and the filtrate was concentrated and purified by silica gel column chromatography (PE:EA=15:1) to give tert-butyl 3-(4-amino-3-fluorophenoxy)-1H-pyrazole-1-carboxylate (15.4 g, 24%) as a white solid. LC-MS: 294.1 [M+H]⁺.

### Step 4: Preparation of 4-((1H-pyrazol-3-yl)oxy)-2-fluoroaniline

To a solution of tert-butyl 3-(4-amino-3-fluorophenoxy)-1H-pyrazole-1-carboxylate (9.4 g, 31.94 mmol) in DCM (40 mL) was added TFA (20 mL) at 25° C. The reaction was stirred at 25° C. for 2 h. The solvent was removed and the residue was dissolved with water.

The mixture was adjusted to pH=9 with sat. NaHCO₃, and extracted with EA (200 mL×3). The organic layer was concentrated to give 4-((1H-pyrazol-3-yl)oxy)-2-fluoroaniline (6.4 g, 83%) as a yellow solid. LC-MS: 194.7 [M+H]⁺.

### Step 5: Preparation of 2-fluoro-4-((1-(2-methoxypyrimidin-5-yl)-1H-pyrazol-3-yl)oxy)aniline

A mixture of 2-fluoro-4-(1H-pyrazol-3-yloxy)aniline (8.85 g, 46 mmol), 5-iodo-2-methoxypyrimidine (12.97 g, 55 mmol), trans N1,N2-dimethylcyclohexane-1,2-diamine (6.51 g, 46 mmol), Copper(I) iodide (8.72 g, 46 mmol) and K₂CO₃ (12.66 g, 92 mmol) in DMSO (360 mL) was stirred at 100° C. for 16 h under N₂ atmosphere. The mixture was diluted with water (100 mL), extracted with EA (100 mL×3). The organic phase was washed with brine (50 mL), dried over Na₂SO₄ and concentrated in vacuo. The crude material was purified by flash column chromatography (PE:EA=100:40) to give 2-fluoro-4-((1-(2-methoxypyrimidin-5-yl)-1H-pyrazol-3-yl)oxy)aniline (6 g, 41%) as a white solid. LC-MS: 302.7 [M+H]⁺.

### Step 6: Preparation of 6-bromo-N-(2-fluoro-4-((1-(2-methoxypyrimidin-5-yl)-1H-pyrazol-3-yl)oxy)phenyl)-7-methoxyquinazolin-4-amine

To a solution of 2-fluoro-4-{[1-(2-methoxypyrimidin-5-yl)pyrazo]-3-yl]oxy}aniline (2 g, 6.64 mmol) and 6-bromo-4-chloro-7-methoxyquinazoline (1.8 g, 6.64 mmol) in dioxane (40 mL) was added HCl/dioxane (4N, 0.2 mL). The mixture was stirred at 25° C. for 16 h. The reaction mixture was concentrated, diluted with DCM. Then Et₃N (2 mL) was added and the mixture was stirred for 5 min. The solid was collected by filtration and washed with DCM/MeOH (25/1) and dried in vacuo to give 6-bromo-N-(2-fluoro-4-((1-(2-methoxypyrimidin-5-yl)-1H-pyrazol-3-yl)oxy)phenyl)-7-methoxyquinazolin-4-amine (3 g, 80%) as a white solid. LC-MS: 538.0, 540.0 [M+H]⁺.

### Step 7: Preparation of tert-butyl (S)-3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidine-1-carboxylate

To a solution of 6-bromo-N-(2-fluoro-4-((1-(2-methoxypyrimidin-5-yl)-1H-pyrazol-3-yl)oxy)phenyl)-7-methoxyquinazolin-4-amine (150 mg, 0.28 mmol) in toluene (10 mL) was added tert-butyl (S)-3-aminopyrrolidine-1-carboxylate (51.8 mg, 0.278 mmol), Brettphos Pd G3 (25.26 mg) and t-BuONa (53 mg). The reaction mixture was stirred at 100° C. for 16 hours and then concentrated and purified by Combi-Flash (MeOH/DCM=0-5%) to afford tert-butyl (S)-3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidine-1-carboxylate (100 mg, 56%) as a yellow solid. LC/MS: 644.6[M+H]⁺.

### Step 8: Preparation of (S)—N4-(2-fluoro-4-((1-(2-methoxypyrimidin-5-yl)-1H-pyrazol-3-yl)oxy)phenyl)-7-methoxy-N6-(pyrrolidin-3-yl)quinazoline-4,6-diamine

To a solution of tert-butyl (S)-3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidine-1-carboxylate (100 mg, 0.16 mmol) in DCM (12 mL) was added TFA (2.4 mL). The mixture was stirred at rt for 30 min. The mixture was poured into ice NaHCO₃ (10 mL) and extracted with DCM (10 mL×3). The organic layer was dried over Na₂SO₄ and concentrated. The resulting residue (70 mg) was used for next step without further purification. LC/MS: 544.5 [M+H]⁺.

### Step 9: Preparation of (S)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one (8)

To a solution of (S)—N4-(2-fluoro-4-((1-(2-methoxypyrimidin-5-yl)-1H-pyrazol-3-yl)oxy)phenyl)-7-methoxy-N6-(pyrrolidin-3-yl)quinazoline-4,6-diamine (70 mg, 0.13 mmol) in DCM (10 mL) was added DIEA (53.5 mg, 0.414 mmol) and prop-2-enoyl chloride (10 mg, 0.11 mmol). The reaction mixture was stirred for 15 min at −78° C. The solution was quenched with MeCN and water. The mixture was concentrated and purified by Prep-HPLC (0.1% TFA in water/MeCN from 70% to 25%) to afford (S)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one (44.5 mg, 58%) as a yellow solid. LC/MS: 598.6[M+H]⁺; ¹H NMR (400 MHz, CD₃OD) δ8.94 (s, 2H), 8.54 (d, J=6.0 Hz, 1H), 8.26 (d, J=6.0 Hz, 1H), 7.55 (t, J=12 Hz, 1H), 7.42 (d, J=8.0 Hz, 1H), 7.25-7.14 (m, 3H), 6.70-6.58 (m, 1H), 6.33-6.26 (m, 2H), 5.79-5.73 (m, 1H), 4.38-4.30 (m, 1H), 4.15 (s, 3H), 4.05 (s, 3H), 3.99-3.92 (m, 1H), 3.90-3.80 (m, 1H), 3.71-3.59 (m, 2H), 2.49-2.47 (m, 1H), 2.25-2.24 (m, 1H).

### Example 9: Preparation of (R)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one (Compound 9)

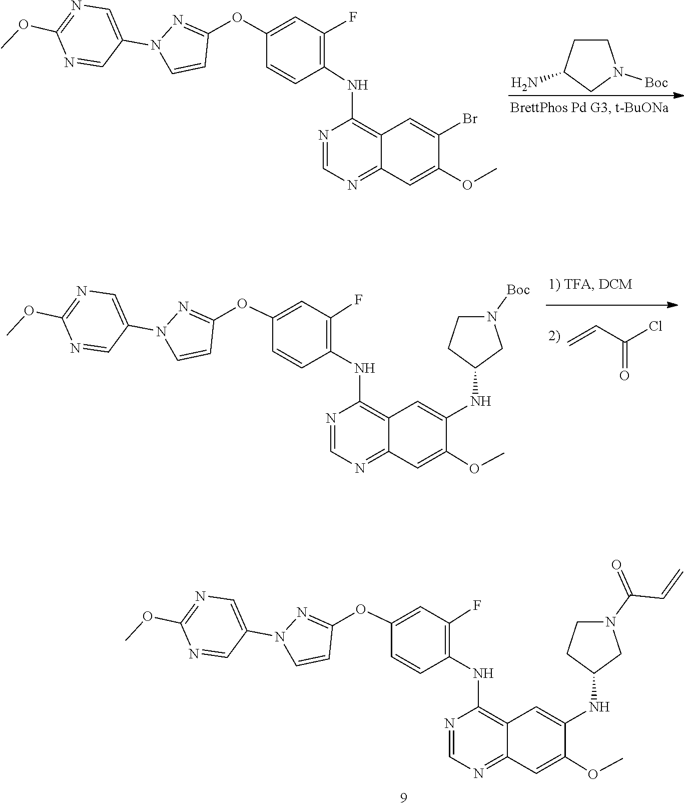

This compound was prepared using the same procedure as described in Example 8. The crude product was purified by preparative HPLC (0.1% TFA in water/MeCN from 70% to 25%) to give (R)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one as a yellow solid. LC/MS: 598.6[M+H]⁺; ¹H NMR (400 MHz, CD₃OD) δ8.94 (s, 2H), 8.54 (d, J=6.0 Hz, 1H), 8.26 (d, J=6.0 Hz, 1H), 7.55 (t, J=12 Hz, 1H), 7.42 (d, J=8.0 Hz, 1H), 7.25-7.14 (m, 3H), 6.70-6.58 (m, 1H), 6.33-6.26 (m, 2H), 5.79-5.73 (m, 1H), 4.38-4.30 (m, 1H), 4.15 (s, 3H), 4.05 (s, 3H), 3.99-3.92 (m, 1H), 3.90-3.80 (m, 1H), 3.71-3.59 (m, 2H), 2.49-2.47 (m, 1H), 2.25-2.24 (m, 1H).

### Example 10: Preparation of 1-(4-((4-((4-((1-(5-chloro-6-methylpyridin-3-yl)-1H-pyrazol-3-yl)oxy)-2-fluorophenyl)amino)-7-methoxyquinazolin-6-yl)amino)piperidin-1-yl)prop-2-en-1-one (Compound 10)

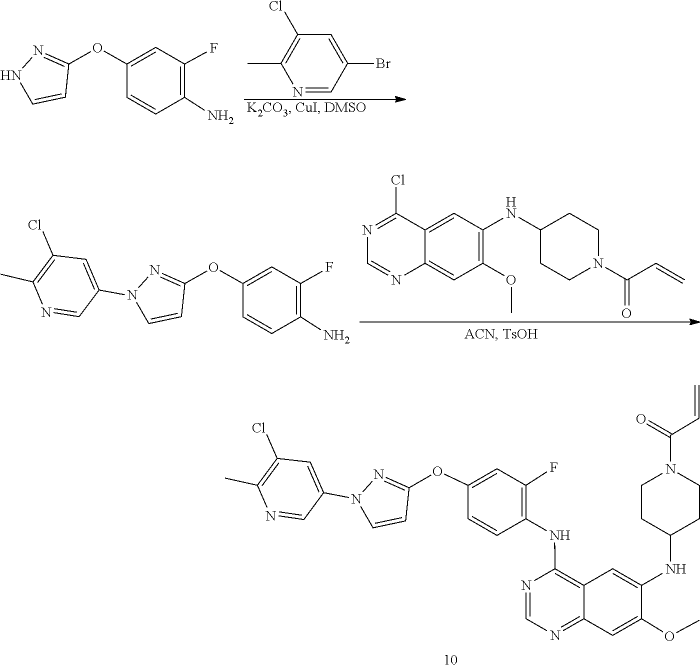

### Step 1: Preparation of 4-((1-(5-chloro-6-methylpyridin-3-yl)-1H-pyrazol-3-yl)oxy)-2-fluoroaniline

To a solution of 2-fluoro-4-(1H-pyrazol-3-yloxy)aniline (200 mg, 1.04 mmol) and K₂CO₃ (286 mg, 2.07 mmol) in DMSO (5 mL) was added 5-bromo-3-chloro-2-methylpyridine (256 mg, 1.24 mmol), (1S,2S)-1-N,2-N-dimethylcyclohexane-1,2-diamine (147 mg, 1.04 mmol) and CuI (197 mg, 1.04 mmol). The reaction mixture was stirred at 100° C. for 16 hours. The mixture was diluted with EtOAc, washed with brine, dried over Na₂SO₄, filtered and the filtrate was concentrated under vacuum. The crude residue was purified by Combi-Flash (eluted by EA/PE=0-40%) to give 4-((1-(5-chloro-6-methylpyridin-3-yl)-1H-pyrazol-3-yl)oxy)-2-fluoroaniline (150 mg, 45%) as a yellow solid. LC/MS: 318.9 [M+H]⁺.

### Step 2: Preparation of 1-(4-((4-((4-((1-(5-chloro-6-methylpyridin-3-yl)-1H-pyrazol-3-yl)oxy)-2-fluorophenyl)amino)-7-methoxyquinazolin-6-yl)amino)piperidin-1-yl)prop-2-en-1-one (10)

To a mixture of 4-{[1-(5-chloro-6-methylpyridin-3-yl)pyrazo]-3-yl]oxy}-2-fluoroaniline (46 mg, 0.14 mmol) and 1-{4-[(4-chloro-7-methoxyquinazolin-6-yl)amino]piperidin-1-yl}prop-2-en-1-one (250 mg, 20% purity, 0.14 mmol) in CH₃CN (3 mL) was added TsOH (2.48 mg, 0.014 mmol). The reaction was stirred at 75° C. for 2 hr. After concentration under vacuum, the crude mixture was purified by Prep-HPLC, using a gradient of 0.1% TFA/ACN from 70% to 10% to give 1-[4-({4-[(4-{[1-(5-chloro-6-methylpyridin-3-yl)pyrazo]-3-yl]oxy}-2-fluorophenyl)amino]-7-methoxyquinazolin-6-yl}amino)piperidin-1-yl]prop-2-en-1-one (37 mg, 41%) as a yellow solid. LC/MS: 629.2[M+H]⁺; ¹H NMR (400 MHz, CD₃OD) δ8.82 (d, J=2.3 Hz, 1H), 8.36 (d, J=2.7 Hz, 1H), 8.28 (s, 1H), 8.23 (d, J=2.2 Hz, 1H), 7.58 (t, J=8.7 Hz, 1H), 7.32 (s, 1H), 7.25-7.14 (m, 2H), 7.10 (s, 1H), 6.84 (dd, J=16.8, 10.7 Hz, 1H), 6.29-6.18 (m, 2H), 5.77 (dd, J=10.7, 2.0 Hz, 1H), 4.60 (d, J=12.0 Hz, 1H), 4.21 (d, J=16.0 Hz, 1H), 4.19 (s, 3H), 3.92-3.80 (m, 1H), 3.39 (d, J=11.6 Hz, 1H), 3.03 (t, J=11.9 Hz, 1H), 2.62 (s, 3H), 2.30-2.20 (m, 2H), 1.61-1.48 (m, 2H).

### Example 11: Preparation of 1-(4-((4-((2-fluoro-4-((1-(6-methoxypyridin-3-yl)-1H-pyrazol-3-yl)oxy)phenyl)amino)-7-methoxyquinazolin-6-yl)amino)piperidin-1-yl)prop-2-en-1-one (Compound 12)

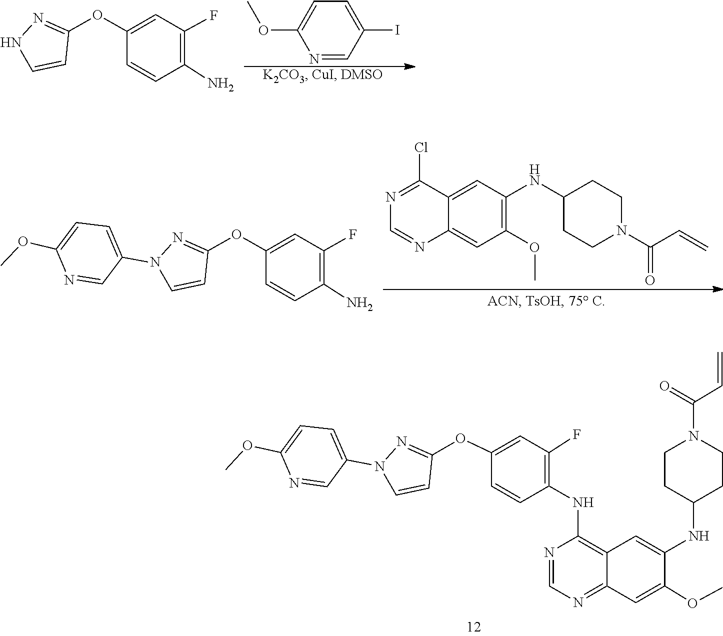

### Step 1: Preparation of 2-fluoro-4-((1-(6-methoxypyridin-3-yl)-1H-pyrazol-3-yl)oxy)aniline

To a solution of 2-fluoro-4-(1H-pyrazol-3-yloxy)aniline (200 mg, 1.04 mmol) and K₂CO₃ (286 mg, 2.07 mmol) in DMSO (5 mL) was added 5-iodo-2-methoxypyridine (292 mg, 1.24 mmol), (1S,2S)-1-N,2-N-dimethylcyclohexane-1,2-diamine (147 mg, 1.04 mmol) and CuI (197 mg, 1.04 mmol). The reaction mixture was stirred at 100° C. for 16 hours. The mixture was diluted with EtOAc, washed with brine, dried over Na₂SO₄, filtered and the filtrate was concentrated under vacuum. The crude residue was purified by Combi-Flash (eluted by EA/PE=0-40%) to give 2-fluoro-4-((1-(6-methoxypyridin-3-yl)-1H-pyrazol-3-yl)oxy)aniline (150 mg, 48%) as a yellow solid. LC/MS: 300.9 [M+H]⁺.

### Step 2: Preparation of 1-(4-((4-((2-fluoro-4-((1-(6-methoxypyridin-3-yl)-1H-pyrazol-3-yl)oxy)phenyl)amino)-7-methoxyquinazolin-6-yl)amino)piperidin-1-yl)prop-2-en-1-one (12)

To a mixture of 2-fluoro-4-{[1-(6-methoxypyridin-3-yl)pyrazo]-3-yl]oxy}aniline (43 mg, 0.14 mmol) and 1-{4-[(4-chloro-7-methoxyquinazolin-6-yl)amino]piperidin-1-yl}prop-2-en-1-one (250 mg, 20% purity, 0.14 mmol) in CH₃CN (3 mL) was added TsOH (2.48 mg, 0.014 mmol). The reaction was stirred at 75° C. for 2 hr. The reaction was concentrated under vacuum. The crude residue was purified by Prep-HPLC using a gradient of 0.1% TFA/ACN from 70% to 10%, to give 1-(4-((4-((2-fluoro-4-((1-(6-methoxypyridin-3-yl)-1H-pyrazol-3-yl)oxy)phenyl)amino)-7-methoxyquinazolin-6-yl)amino)piperidin-1-yl)prop-2-en-1-one (40 mg, 46%) as a yellow solid. LC/MS: 611.3 [M+H]⁺; ¹H NMR (400 MHz, CD₃OD) δ8.51 (d, J=2.8 Hz, 1H), 8.28 (d, J=7.4 Hz, 1H), 8.17 (d, J=2.6 Hz, 1H), 8.04 (dd, J=8.9, 2.8 Hz, 1H), 7.56 (t, J=8.7 Hz, 1H), 7.32 (s, 1H), 7.20-7.04 (m, 3H), 6.93 (d, J=8.9 Hz, 1H), 6.83 (dd, J=16.8, 10.7 Hz, 1H), 6.30-6.14 (m, 2H), 5.77 (dd, J=10.7, 2.0 Hz, 1H), 4.60 (d, J=13.0 Hz, 1H), 4.21 (d, J=14.8 Hz, 1H), 4.06 (s, 3H), 3.95 (s, 3H), 3.89-3.80 (m, 1H), 3.38 (d, J=12.7 Hz, 1H), 3.02 (t, J=12.4 Hz, 1H), 2.29-2.17 (m, 2H), 1.61-1.46 (m, 2H).

### Example 12: Preparation of 5-(3-(4-((6-((1-acryloylpiperidin-4-yl)amino)-7-methoxyquinazolin-4-yl)amino)-3-fluorophenoxy)-1H-pyrazol-1-yl)-2-methylnicotinonitrile (Compound 11)

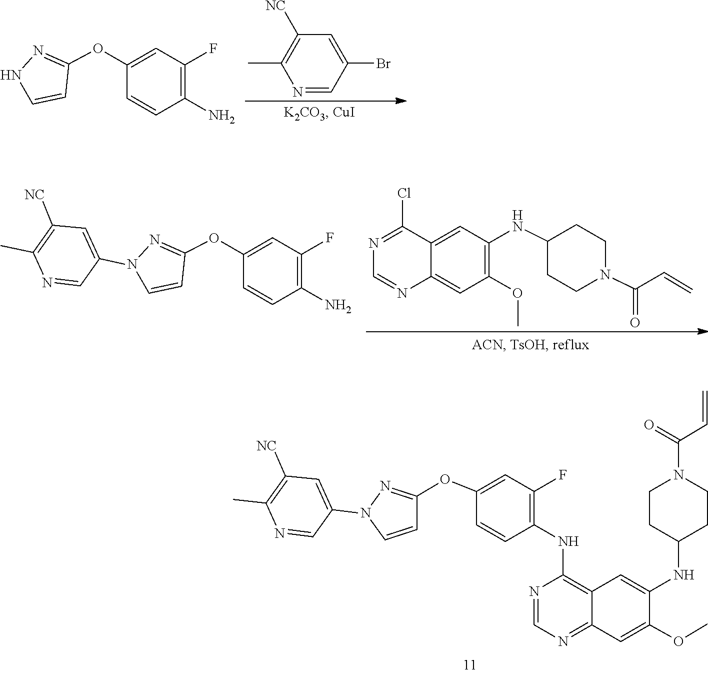

### Step 1: Preparation of 5-(3-(4-amino-3-fluorophenoxy)-1H-pyrazol-1-yl)-2-methylnicotinonitrile

To a solution of 2-fluoro-4-(1H-pyrazol-3-yloxy)aniline (70 mg, 0.36 mmol) in DMSO (5 mL) was added 5-bromo-2-methylpyridine-3-carbonitrile (86 mg, 0.43 mmol), trans-1-N,2-N-dimethylcyclohexane-1,2-diamine (52 mg, 0.36 mmol), K₂CO₃ (100 mg, 0.72 mmol) and CuI (69 mg, 0.36 mmol). The reaction mixture was stirred at 100° C. for 16 hours under N₂ atmosphere. The mixture was diluted with EtOAc, washed with brine, dried over Na₂SO₄, filtered and the filtrate was concentrated and purified by Combi-Flash (eluted by EA/PE=0-40%) to give 5-(3-(4-amino-3-fluorophenoxy)-1H-pyrazol-1-yl)-2-methylnicotinonitrile (72 mg, 65%) as a yellow solid. LC/MS: 310.0[M+H]⁺.

### Step 2: Preparation of 5-(3-(4-((6-((1-acryloylpiperidin-4-yl)amino)-7-methoxyquinazolin-4-yl)amino)-3-fluorophenoxy)-1H-pyrazol-1-yl)-2-methylnicotinonitrile (11)

To a mixture of 5-(3-(4-amino-3-fluorophenoxy)-1H-pyrazol-1-yl)-2-methylnicotinonitrile (45 mg, 0.15 mmol) and 1-{4-[(4-chloro-7-methoxyquinazolin-6-yl)amino]piperidin-1-yl}prop-2-en-1-one (50 mg, 0.15 mmol) in CH₃CN (2 mL) was added TsOH (2.5 mg, 0.015 mmol). The reaction was stirred at 75° C. for 2 h. The reaction mixture was concentrated and purified by Prep-HPLC, using a gradient of 0.1% FA/ACN from 80% to 60% to give 5-(3-(4-((6-((1-acryloylpiperidin-4-yl)amino)-7-methoxyquinazolin-4-yl)amino)-3-fluorophenoxy)-1H-pyrazol-1-yl)-2-methylnicotinonitrile (43.2 mg, 47%) as a yellow solid. LC/MS: 619.8[M+H]⁺. ¹H NMR (400 MHz, CD₃OD) δ9.12 (d, J=2.7 Hz, 1H), 8.48 (d, J=2.6 Hz, 1H), 8.39 (d, J=2.7 Hz, 1H), 8.32 (s, 1H), 8.24 (brs, 1H, FA), 7.59 (t, J=8.7 Hz, 1H), 7.33 (s, 1H), 7.25-7.16 (m, 2H), 7.11 (s, 1H), 6.84 (dd, J=16.8, 10.7 Hz, 1H), 6.31 (d, J=2.7 Hz, 1H), 6.23 (dd, J=16.8, 2.0 Hz, 1H), 5.77 (dd, J=10.7, 2.0 Hz, 1H), 4.62-4.58 (m, 1H), 4.23-4.20 (m, 1H), 4.08 (s, 3H), 3.90-3.82 (m, 1H), 3.41-3.37 (m, 1H), 3.04-3.01 (m, 1H), 2.78 (s, 3H), 2.29-2.20 (m, 2H), 1.62-1.49 (m, 2H).

### Example 13: Preparation of 1-(4-((7-ethoxy-4-((2-fluoro-4-((1-(5-fluoro-6-methylpyridin-3-yl)-1H-pyrazol-3-yl)oxy)phenyl)amino)quinazolin-6-yl)amino)piperidin-1-yl)prop-2-en-1-one (Compound 13)

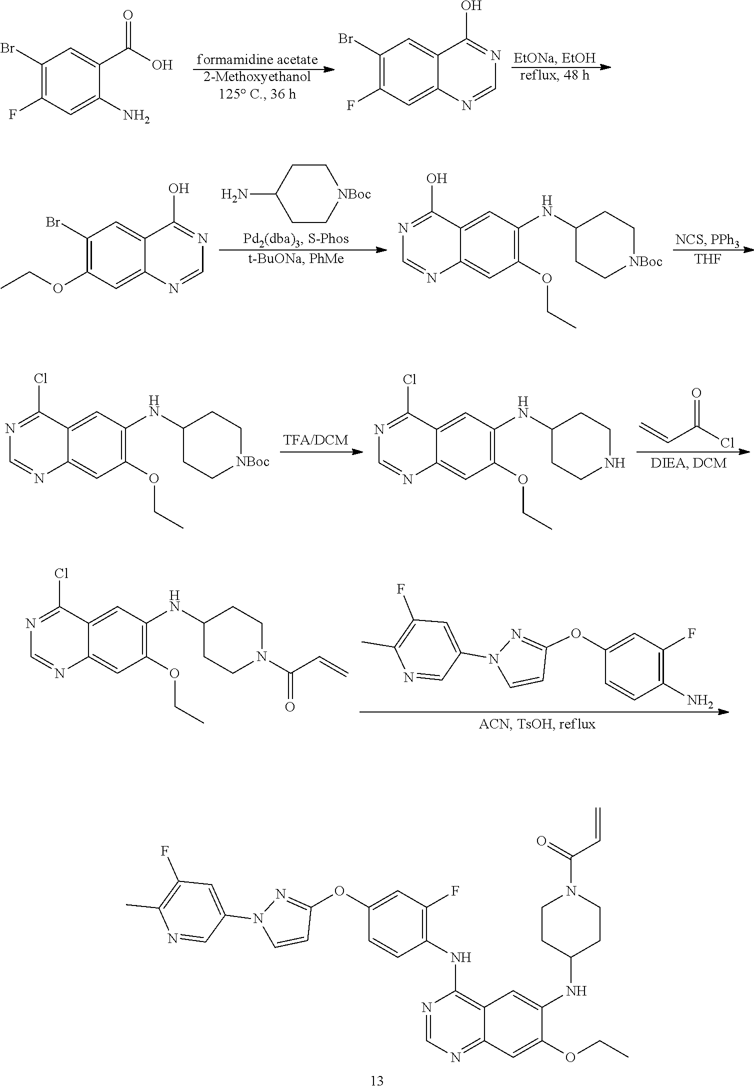

### Step 1: Preparation of 6-bromo-7-fluoroquinazolin-4-ol

To a solution of 2-amino-5-bromo-4-fluorobenzoic acid (11.8 g, 50.86 mmol) in 2-methoxyethanol (100 mL) was added formamidine acetate (23.1 g, 222.2 mmol). The mixture was stirred at 125° C. for 36 h. The mixture was filtered and the solid was lyophilized to give 6-bromo-7-fluoroquinazolin-4-ol (12 g, 97%) as a white solid. LC/MS: 243.0 [M+H]⁺.

### Step 2: Preparation of 6-bromo-7-ethoxyquinazolin-4-ol

To a solution of sodium ethoxide in EtOH (20%, 20 mL) was added 6-bromo-7-fluoroquinazolin-4-ol (2 g, 8.23 mmol). The mixture was stirred at 85° C. for 16 h. The mixture was concentrated in vacuo and diluted with water. The solution was adjusted to pH=3 with HCl and filtered. The wet cake was collected and lyophilized for 48 h to give 6-bromo-7-ethoxyquinazolin (2 g, 91%) as a yellow solid. LC/MS:268.8 [M+H]⁺.

### Step 3: Preparation of tert-butyl 4-((7-ethoxy-4-hydroxyquinazolin-6-yl)amino)piperidine-1-carboxylate

To a solution of 6-bromo-7-ethoxyquinazolin-4-ol (1 g, 3.72 mmol) in toluene (15 mL) was added tert-butyl 4-aminopiperidine-1-carboxylate (893 mg, 4.46 mmol), Pd₂(dba)₃ (340 mg, 0.37 mmol), t-BuONa (1 g, 11.15 mmol) and S-phos (305 mg, 0.74 mmol). The mixture was stirred at 100° C. for 16 h. The mixture was concentrated and the residue was purified by Combi-Flash (DCM/MeOH=10/1) to afford the titled product (850 mg, 59%) as a yellow solid. LC/MS:388.9 [M+H]⁺.

### Step 4: Preparation of tert-butyl 4-((4-chloro-7-ethoxyquinazolin-6-yl)amino)piperidine-1-carboxylate

To a solution of N-chlorosuccinimide (877 mg, 6.56 mmol) in THE (10 mL) was added PPh₃ (1.7 g, 6.56 mmol). The mixture was stirred at 20° C. for 1 h. A solution of tert-butyl 4-[(7-ethoxy-4-hydroxyquinazolin-6-yl)amino]piperidine-1-carboxylate (850 mg, 2.19 mmol) in THE was added. The reaction mixture was stirred at 50° C. for 16 h. The mixture was quenched with triethylamine and stirred for 1 h. The mixture was concentrated and the residue was purified by Combi-Flash (PE/EA=1/1) to afford the product (2 g, purity: 27%, 61%) as a yellow solid. LC/MS:407.1 [M+H]⁺.

### Step 5: Preparation of 4-chloro-7-ethoxy-N-(piperidin-4-yl)quinazolin-6-amine

A solution of tert-butyl 4-[(4-chloro-7-ethoxyquinazolin-6-yl)amino]piperidine-1-carboxylate (2 g, crude) in DCM/TFA=3:1 (8 mL) was stirred at 20° C. for 2 h. The mixture was concentrated to afford the product (3.3 g, crude) as a yellow oil. LC/MS:307.0 [M+H]⁺.

### Step 6: Preparation of 1-(4-((4-chloro-7-ethoxyquinazolin-6-yl)amino)piperidin-1-yl)prop-2-en-1-one

To a solution of 4-chloro-7-ethoxy-N-(piperidin-4-yl)quinazolin-6-amine (3.3 g, crude) in DCM (28 mL) was added prop-2-enoyl chloride (250 mg, 28 mmol) and DIEA (3.6 g, 28 mmol). The mixture was stirred at −78° C. for 30 min. The reaction mixture was diluted with DCM. The organic layer was washed with brine, dried over Na₂SO₄, filtered and the filtrate was concentrated and purified by Combi-Flash (EA) to afford the product (260 mg, purity: 84%, 22%) as a yellow oil. LC/MS:360.9 [M+H]⁺.

### Step 7: Preparation of 1-(4-((7-ethoxy-4-((2-fluoro-4-((1-(5-fluoro-6-methylpyridin-3-yl)-1H-pyrazol-3-yl)oxy)phenyl)amino)quinazolin-6-yl)amino)piperidin-1-yl)prop-2-en-1-one (13)

To a mixture of 2-fluoro-4-{[1-(5-fluoro-6-methylpyridin-3-yl)pyrazo]-3-yl]oxy}aniline (50 mg, 0.17 mmol) and 1-{4-[(4-chloro-7-ethoxyquinazolin-6-yl)amino]piperidin-1-yl}prop-2-en-1-one (73 mg, 84%, 0.17 mmol) in CH₃CN (2 mL) was added TsOH (2.9 mg, 0.016 mmol). The reaction was stirred at 75° C. for 2 h. The reaction mixture was concentrated and purified by Prep-HPLC (using a gradient of 0.1% FA/ACN from 20% to 50%) to give 1-(4-((7-ethoxy-4-((2-fluoro-4-((1-(5-fluoro-6-methylpyridin-3-yl)-1H-pyrazol-3-yl)oxy)phenyl)amino)quinazolin-6-yl)amino)piperidin-1-yl)prop-2-en-1-one (29.7 mg, 24%) as a yellow solid. LC/MS: 627.1 [M+H]⁺. ¹H NMR (400 MHz, CD3OD) δ 8.72 (d, J=1.9 Hz, 1H), 8.33 (d, J=2.7 Hz, 1H), 8.23 (s, 1H), 7.95 (dd, J=10.4, 2.0 Hz, 1H), 7.56 (t, J=8.7 Hz, 1H), 7.29 (s, 1H), 7.18 (dd, J=11.1, 2.6 Hz, 1H), 7.15-7.11 (m, 1H), 7.06 (s, 1H), 6.81 (dd, J=16.8, 10.7 Hz, 1H), 6.25 (d, J=2.7 Hz, 1H), 6.21 (dd, J=16.8, 2.0 Hz, 1H), 5.75 (dd, J=10.7, 2.0 Hz, 1H), 4.58 (d, J=13.6 Hz, 1H), 4.31-4.24 (m, 2H), 4.19 (d, J=14.3 Hz, 1H), 3.86-3.80 (m, 1H), 3.40-3.33 (m, 1H), 3.00 (t, J=11.5 Hz, 1H), 2.52 (s, 3H), 2.27-2.19 (m, 2H), 1.53 (t, J=7.0 Hz, 5H).

### Example 14: Preparation of 1-[4-({4-[(2-fluoro-4-{[1-(5-methylpyridin-2-yl)pyrazo]-3-yl]oxy}phenyl)amino]-7-methoxyquinazolin-6-yl}amino)piperidin-1-yl]prop-2-en-1-one (Compound 14)

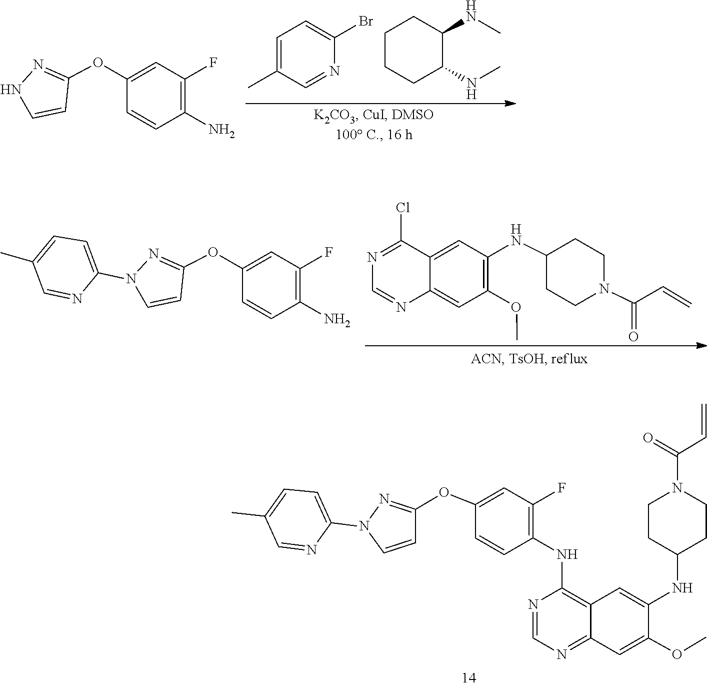

### Step 1: 2-fluoro-4-{[1-(5-methylpyridin-2-yl)pyrazo]-3-yl]oxy}aniline

To a mixture of 2-fluoro-4-(1H-pyrazol-3-yloxy)aniline (200 mg, 1.04 mmol) and K₂CO₃ (286 mg, 2.08 mmol) in DMSO (5 mL) was added 2-bromo-5-methylpyridine (214 mg, 1.24 mmol), (1S,2S)-1-N,2-N-dimethylcyclohexane-1,2-diamine (147 mg, 1.04 mmol), CuI (197 mg, 1.04 mmol). The reaction mixture was stirred at 100° C. for 16 h. The mixture was diluted with EtOAc, washed with brine, dried over Na₂SO₄, filtered and the filtrate was concentrated and purified by Combi-Flash (eluted by EA/PE=0-40%) to give 2-fluoro-4-{[1-(5-methylpyridin-2-yl)pyrazo]-3-yl]oxy}aniline (210 mg, 67%) as a yellow solid. LC/MS: 284.9[M+H]⁺.

### Step 2: 1-[4-({4-[(2-fluoro-4-{[1-(5-methylpyridin-2-yl)pyrazo]-3-yl]oxy}phenyl)amino]-7-methoxyquinazolin-6-yl}amino)piperidin-1-yl]prop-2-en-1-one (14)

To a mixture of 2-fluoro-4-{[1-(4-fluoro-5-methylpyridin-2-yl)pyrazo]-3-yl]oxy}aniline (50 mg, 0.18 mmol) and 1-{4-[(4-chloro-7-methoxyquinazolin-6-yl)amino]piperidin-1-yl}prop-2-en-1-one (60 mg, 0.18 mmol) in CH3CN (3 mL) was added TsOH (3 mg, 0.018 mmol). The reaction was stirred for at 75° C. 2 h. The reaction was concentrated and the crude residue was purified by Prep-HPLC, using a gradient of 0.1% TFA/ACN from 70% to 10% to give 1-[4-({4-[(2-fluoro-4-{[1-(5-methylpyridin-2-yl)pyrazo]-3-yl]oxy}phenyl)amino]-7-methoxyquinazolin-6-yl}amino)piperidin-1-yl]prop-2-en-1-one (36.7 mg, 36%) as a yellow solid. LC/MS: 595.0[M+H]⁺. ¹H NMR (400 MHz, CD₃OD) δ8.57-8.55 (m, 2H), 8.28 (s, 1H), 7.74-7.76 (m, 2H), 7.58 (t, J=8.6 Hz, 1H), 7.45 (s, 1H), 7.20-7.22 (m, 3H), 6.84 (dd, J=16.8, 10.7 Hz, 1H), 6.25-6.23 (m, 2H), 5.78 (dd, J=10.7, 1.9 Hz, 1H), 4.67-4.58 (m, 1H), 4.28-4.20 (m, 1H), 4.14 (s, 3H), 3.91-3.81 (m, 1H), 3.42-3.34 (m, 1H), 3.05-2.96 (m, 1H), 2.39 (s, 3H), 2.20-2.25 (m, 2H). 1.55-1.65 (m, 2H).

### Example 15: Preparation of 1-(4-((4-((2-fluoro-4-((1-(4-fluoro-5-methylpyridin-2-yl)-1H-pyrazol-3-yl)oxy)phenyl)amino)-7-methoxyquinazolin-6-yl)amino)piperidin-1-yl)prop-2-en-1-one (Compound 15)

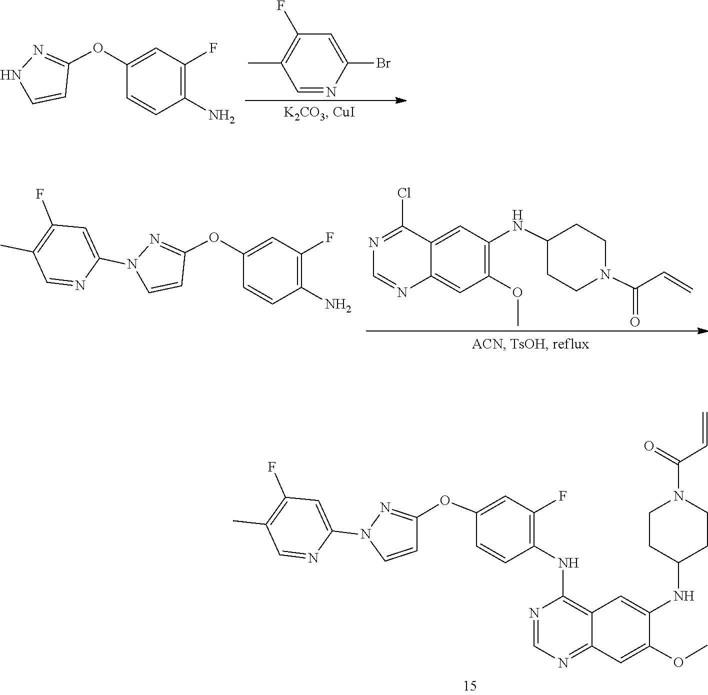

### Step 1: Preparation of 2-fluoro-4-((1-(4-fluoro-5-methylpyridin-2-yl)-1H-pyrazol-3-yl)oxy)aniline

To a solution of 2-fluoro-4-(1H-pyrazol-3-yloxy)aniline (50 mg, 0.25 mmol) in DMSO (2 mL) was added 2-bromo-4-fluoro-5-methylpyridine (60 mg, 0.31 mmol), Trans-N,N′-dimethyl-cyclohexane-1,2-diamine (37 mg, 0.25 mmol), CuI (49 mg, 0.25 mmol) and K₂CO₃ (36 mg, 0.25 mmol). The reaction mixture was stirred at 100° C. for 16 hours under N₂ atmosphere. The crude reaction mixture was diluted with EtOAc, washed with brine, dried over Na₂SO₄, filtered and the filtrate was concentrated and purified by Combi-Flash (eluted by EA/PE=0-40%) to give 2-fluoro-4-{[1-(4-fluoro-5-methylpyridin-2-yl)pyrazo]-3-yl]oxy}aniline (60 mg, 79%) as a yellow solid. LC/MS: 303.2[M+H]⁺.

### Step 2: Preparation of 1-(4-((4-((2-fluoro-4-((1-(4-fluoro-5-methylpyridin-2-yl)-1H-pyrazol-3-yl)oxy)phenyl)amino)-7-methoxyquinazolin-6-yl)amino)piperidin-1-yl)prop-2-en-1-one (15)

To a solution of 2-fluoro-4-{[1-(4-fluoro-5-methylpyridin-2-yl)pyrazo]-3-yl]oxy}aniline (60 mg, 0.19 mmol) and 1-{4-[(4-chloro-7-methoxyquinazolin-6-yl)amino]piperidin-1-yl}prop-2-en-1-one (69 mg, 0.19 mmol) in CH₃CN (3 mL) was added TsOH (3.41 mg, 0.019 mmol). The reaction was stirred at 75° C. for 2 h. The reaction was concentrated and the crude mixture was purified by Prep-HPLC using a gradient of 0.1% TFA/ACN from 70% to 10% to give 1-[4-({4-[(2-fluoro-4-{[1-(4-fluoro-5-methylpyridin-2-yl)pyrazo]-3-yl]oxy}phenyl)amino]-7-methoxyquinazolin-6-yl}amino)piperidin-1-yl]prop-2-en-1-one (32 mg, 25%) as a yellow solid. LC/MS: 613.3[M+H]⁺. ¹H NMR (400 MHz, CD₃OD) δ8.55 (d, J=2.7 Hz, 1H), 8.31 (d, J=10.0 Hz, 1H), 8.22 (s, 1H, FA), 7.58 (t, J=8.7 Hz, 1H), 7.50 (d, J=10.4 Hz, 1H), 7.29 (s, 1H), 7.26-7.14 (m, 2H), 7.10 (s, 1H), 6.84 (dd, J=16.8, 10.7 Hz, 1H), 6.28-6.17 (m, 2H), 5.77 (dd, J=10.7, 2.0 Hz, 1H), 4.62-4.58 (m, 1H), 4.23-4.20 (m, 1H), 4.05 (s, 3H), 3.90-3.80 (m, 1H), 3.41-3.36 (m, 1H), 3.05-3.02 (m, 1H), 2.35-2.18 (m, 5H), 1.61-1.48 (m, 2H).

### Example 16: Preparation of 1-[4-({4-[(3-chloro-4-{[1-(5-methylpyridin-2-yl)pyrazo]-3-yl]oxy}phenyl)amino]-7-methoxyquinazolin-6-yl}amino)piperidin-1-yl]prop-2-en-1-one (Compound 16)

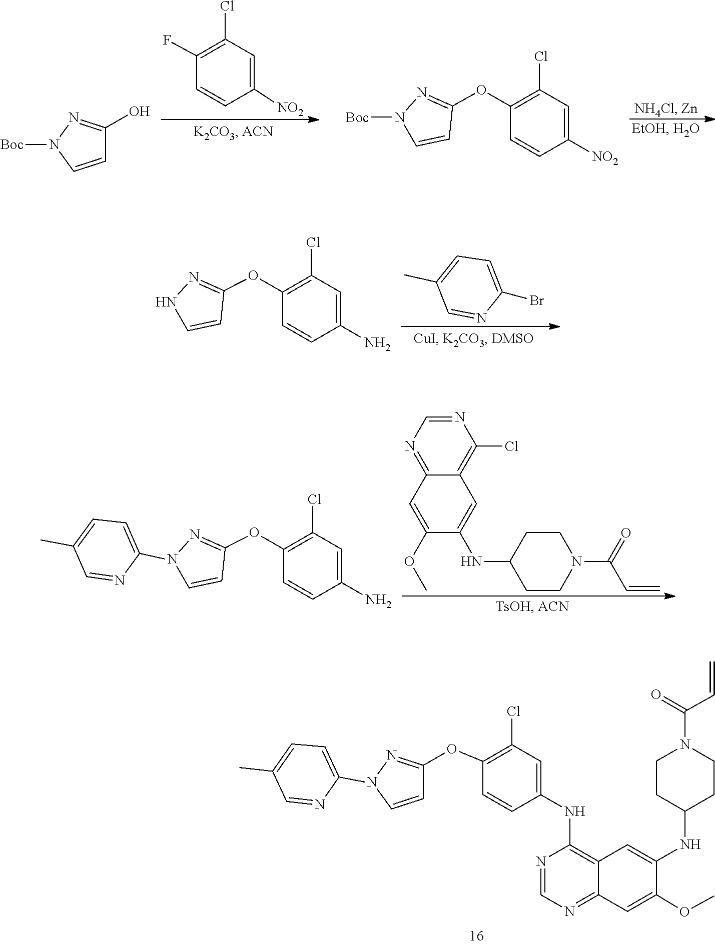

### Step 1: Preparation of tert-butyl 3-(2-chloro-4-nitrophenoxy)-1H-pyrazole-1-carboxylate

To a solution of tert-butyl 3-hydroxy-1H-pyrazole-1-carboxylate (6.33 g, 34.4 mmol) and 2-chloro-1-fluoro-4-nitrobenzene (3 g, 17.2 mmol) in MeCN (170 mL) was added K₂CO₃ (7.12 g, 51.6 mmol). The reaction mixture was stirred at 80° C. for 2 hr. The mixture was concentrated under vacuum and purified by Combi-Flash (PE/EA=10:1) to afford tert-butyl 3-(2-chloro-4-nitrophenoxy)-1H-pyrazole-1-carboxylate (5.5 g, 94%) as a white solid. LC/MS: 284 [M-56]⁺.

### Step 2: Preparation of 4-((1H-pyrazol-3-yl)oxy)-3-chloroaniline

To a solution of tert-butyl 3-(2-chloro-4-nitrophenoxy)-1H-pyrazole-1-carboxylate (2 g, 5.87 mmol) in EtOH (100 mL) and H₂O (20 mL) was added Zn (3.82 g, 58.70 mmol) and ammonium chloride (3.14 g, 58.70 mmol). The reaction mixture was stirred at 80° C. for 2 hr. LC-MS analysis indicated the Boc group was cleaved during reaction. The mixture was filtered and the filtrate was concentrated and purified by Combi-Flash (PE/EA=1:1) to afford 4-((1H-pyrazol-3-yl)oxy)-3-chloroaniline (1.09 g, 89%) as a yellow solid. LC/MS: 210.1 [M+H]⁺.

### Step 3: Preparation of 3-chloro-4-{[1-(5-methylpyridin-2-yl)pyrazo]-3-yl]oxy}aniline

To a suspension of 3-chloro-4-(1H-pyrazol-3-yloxy)aniline (200 mg, 0.95 mmol), 2-bromo-5-methylpyridine (197 mg, 1.14 mmol), 1-N,2-N-dimethylcyclohexane-1,2-diamine (136 mg, 0.95 mmol) and copper(I) iodide (182 mg, 0.95 mmol) in DMSO (8 mL) was added K₂CO₃ (264 mg, 1.91 mmol). The reaction mixture was stirred at 100° C. for 16 h under N₂ atmosphere. Water (60 mL) was added to the reaction mixture and extracted with EtOAC (160 mL). The organic lay was concentrated in vacuo. The residue was purified by Combi-Flash (PE/EA=1:1) to afford 3-chloro-4-{[1-(5-methylpyridin-2-yl)pyrazo]-3-yl]oxy}aniline (50 mg, 18%) as a yellow solid. LC/MS: 301.1 [M+H]⁺.

### Step 4: Preparation of 1-[4-({4-[(3-chloro-4-{[1-(5-methylpyridin-2-yl)pyrazo]-3-yl]oxy}phenyl)amino]-7-methoxyquinazolin-6-yl}amino)piperidin-1-yl]prop-2-en-1-one (16)

To a solution of 3-chloro-4-{[1-(5-methylpyridin-2-yl)pyrazo]-3-yl]oxy}aniline (50 mg, 0.17 mmol) and 1-{4-[(4-chloro-7-methoxyquinazolin-6-yl)amino]piperidin-1-yl}prop-2-en-1-one (58 mg, 0.17 mmol) in MeCN (2 mL) was added TsOH (3 mg, 0.016 mmol). The reaction mixture was stirred at 75° C. for 2 h. Then the reaction was concentrated and purified by Prep-HPLC, using a gradient of 0.1% FA/ACN from 70% to 40% to give 1-[4-({4-[(3-chloro-4-{[1-(5-methylpyridin-2-yl)pyrazo]-3-yl]oxy}phenyl)amino]-7-methoxyquinazolin-6-yl}amino)piperidin-1-yl]prop-2-en-1-one (31.5 mg, 30.4%) as a yellow solid. LC/MS: 610.7 [M+H]⁺. ¹H NMR (400 MHz, DMSO-d6) δ 9.31 (s, 1H), 8.53 (d, J=2.7 Hz, 1H), 8.39 (s, 1H), 8.28 (s, 1H), 8.18 (s, 1H, FA), 8.15 (d, J=2.6 Hz, 1H), 7.83-7.81 (m, 1H), 7.78-7.75 (m, 1H), 7.58 (d, J=8.4 Hz, 1H), 7.42 (d, J=8.9 Hz, 1H), 7.29 (s, 1H), 7.11 (s, 1H), 6.89-6.83 (m, 1H), 6.17 (d, J=2.7 Hz, 1H), 6.14-6.09 (m, 1H), 5.70-5.67 (m, 1H), 5.37 (d, J=8.8 Hz, 1H), 4.47-4.45 (m, 1H), 4.14-4.11 (m, 1H), 3.97 (s, 3H), 3.86-3.83 (m, 1H), 3.27-3.25 (m, 1H), 2.93-2.87 (m, 1H), 2.31 (s, 3H), 2.09-2.05 (m, 2H), 1.48-1.45 (m, 2H).

### Example 17: (2E)-4-(dimethylamino)-1-[4-({4-[(2-fluoro-4-{[1-(5-fluoro-6-methylpyridin-3-yl)pyrazol-3-yl]oxy}phenyl)amino]-7-methoxyquinazolin-6-yl}amino)piperidin-1-yl]but-2-en-1-one (Compound 21)

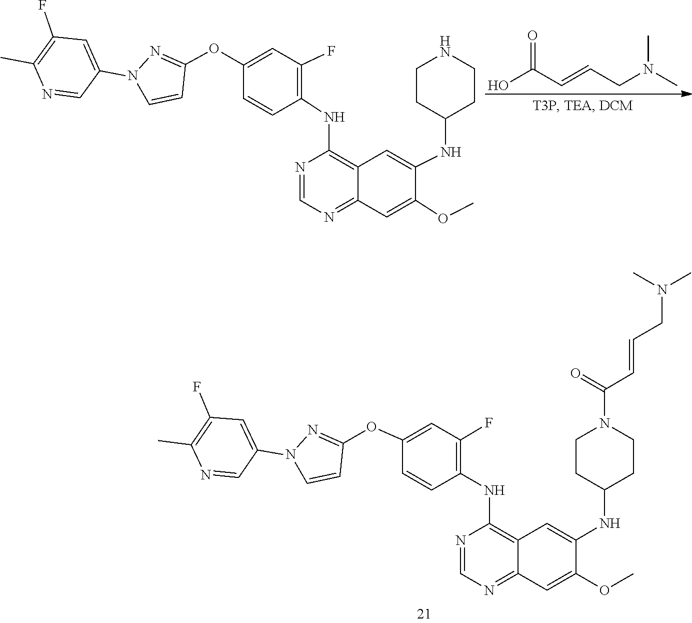

### Step 1: Preparation of (E)-4-(dimethylamino)-1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino) piperidin-1-yl)but-2-en-1-one (21)

A mixture of 4-N-(2-fluoro-4-{[1-(5-fluoro-6-methylpyridin-3-yl)pyrazo]-3-yl]oxy}phenyl)-7-methoxy-6-N-(piperidin-4-yl)quinazoline-4,6-diamine (35 mg, 0.063 mmol), (2E)-4-(dimethylamino)but-2-enoic acid (25 mg, 0.19 mmol), trimethylamine (32 mg, 0.31 mmol) and T₃P (200 mg, 0.31 mmol, 50% in EA) in DCM (20 mL) was stirred at 20° C. for 16 h. The reaction mixture was concentrated under vacuum. The crude residue was purified by Prep-HPLC using a gradient of 0.1% FA/ACN from 75% to 45%, and suitable fractions were pooled and lyophilized to give product (22 mg, 51.83%) as a yellow solid. LC/MS: 669.8 [M+H]⁺. ¹H NMR (400 MHz, CD3OD) δ 8.74 (d, J=2.0 Hz, 1H), 8.41-8.34 (m, 2H), 8.26 (s, 1H, FA), 7.97 (dd, J=10.4, 2.1 Hz, 1H), 7.58-7.56 (m, 1H), 7.31 (s, 1H), 7.22-7.09 (m, 3H), 6.99-6.90 (m, 1H), 6.80-6.68 (m, 1H), 6.28 (d, J=2.7 Hz, 1H), 4.64-4.55 (m, 1H), 4.26-4.16 (m, 1H), 4.06 (s, 3H), 3.91-3.72 (m, 3H), 3.49-3.35 (m, 1H), 3.08-3.02 (m, 1H), 2.78 (s, 6H), 2.54 (d, J=2.9 Hz, 3H), 2.32-2.20 (m, 2H), 1.61-1.46 (m, 2H).

### Example 18: 1-(4-((4-((4-((1-(4,6-dimethoxy-1,3,5-triazin-2-yl)-1H-pyrazol-3-yl)oxy)-2-fluorophenyl)amino)-7-methoxyquinazolin-6-yl)amino)piperidin-1-yl)prop-2-en-1-one (Compound 31)

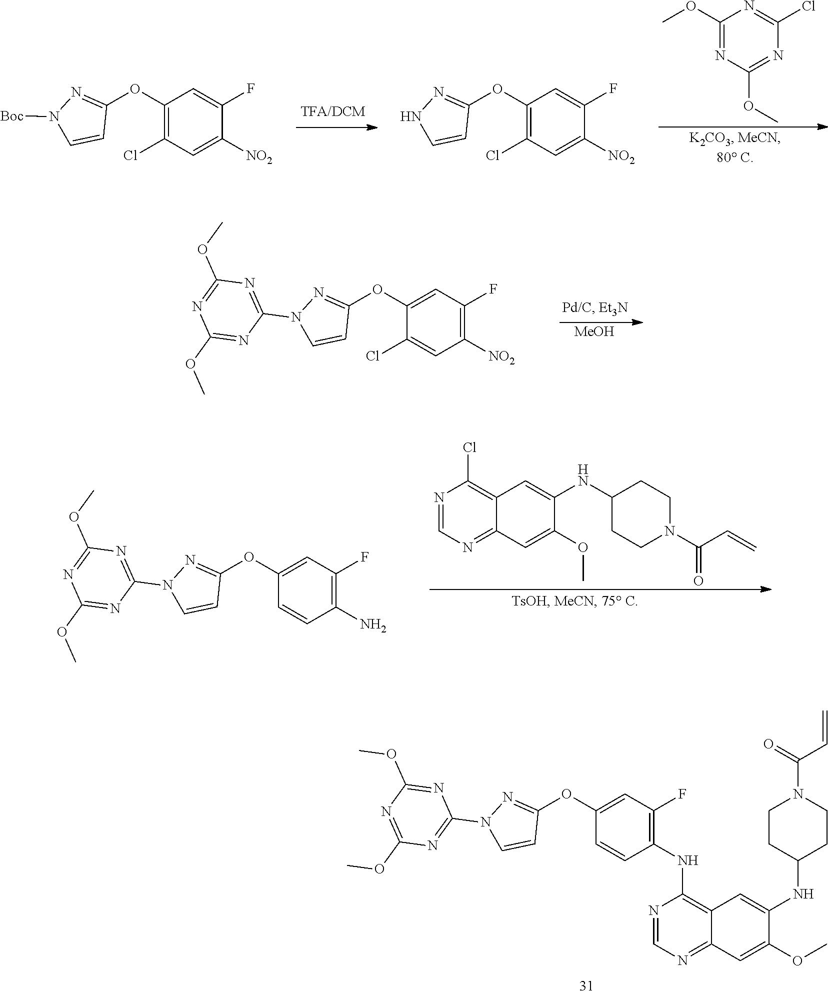

### Step 1: Preparation of 3-(2-chloro-5-fluoro-4-nitrophenoxy)-1H-pyrazole

A solution of tert-butyl 3-(2-chloro-5-fluoro-4-nitrophenoxy)-1H-pyrazole-1-carboxylate (600 mg, 1.67 mmol) in DCM/TFA=3:1 (8 mL) was stirred at 20° C. for 2 h. The crude mixture was diluted with DCM (25 mL), washed with aq. NaHCO₃, dried over Na₂SO₄ and filtered. The filtrate was concentrated and purified by Combi-Flash (eluted by PE/EA=1/1) to give product (250 mg, 58%) as a yellow oil. LC/MS: 257.8[M+H]⁺.

### Step 2: Preparation of 2-(3-(2-chloro-5-fluoro-4-nitrophenoxy)-1H-pyrazol-1-yl)-4,6-dimethoxy-1,3,5-triazine

To a solution of 3-(2-chloro-5-fluoro-4-nitrophenoxy)-1H-pyrazole (250 mg, 0.97 mmol) in MeCN (8 mL) was added 2-chloro-4,6-dimethoxy-1,3,5-triazine (170 mg, 0.97 mmol) and K₂CO₃ (268 mg, 1.94 mmol). The reaction mixture was stirred at 80° C. for 3 hours under N2 atmosphere. The mixture was concentrated and purified by Combi-Flash (eluted by EA/PE=1/2) to give product (160 mg, 41.14%) as a white solid. LC/MS: 396.9 [M+H]⁺.

### Step 3: Preparation of 4-((1-(4,6-dimethoxy-1,3,5-triazin-2-yl)-1H-pyrazol-3-yl)oxy)-2-fluoroaniline

To a solution of 2-[3-(2-chloro-5-fluoro-4-nitrophenoxy)pyrazo]-1-yl]-4,6-dimethoxy-1,3,5-triazine (160 mg, 0.40 mmol) and triethylamine (204 mg, 2.02 mmol) in MeOH (8 mL) was added Pd/C (160 mg). The reaction mixture was stirred at 20° C. for 16 hours under H₂ atmosphere. The mixture was filtered and the filtrate was concentrated and purified by Combi-Flash (eluted by EA/PE=1/1) to give product (30 mg, 22.6%) as a white solid. LC/MS: 332.9 [M+H]⁺.

### Step 4: Preparation of 1-(4-((4-((4-((1-(4,6-dimethoxy-1,3,5-triazin-2-yl)-1H-pyrazol-3-yl)oxy)-2-fluorophenyl)amino)-7-methoxyquinazolin-6-yl)amino)piperidin-1-yl)prop-2-en-1-one (31)

To a mixture of 4-{[1-(4,6-dimethoxy-1,3,5-triazin-2-yl)pyrazo]-3-yl]oxy}-2-fluoroaniline (25 mg, 0.08 mmol) and 1-{4-[(4-chloro-7-methoxyquinazolin-6-yl)amino]piperidin-1-yl}prop-2-en-1-one (26 mg, 0.08 mmol) in MeCN (2 mL) was added TsOH (1.5 mg, 0.008 mmol) carefully. The reaction was stirred at 75° C. for 2 h. The reaction was concentrated and purified by Prep-HPLC (using a gradient of ACN—H₂O (0.05% NH3) from 70% to 20%) to give product (2.4 mg, 4.92%) as a white solid. LC/MS: 643.0 [M+H]⁺; ¹H NMR (400 MHz, CD₃OD) b 8.57 (d, J=2.9 Hz, 1H), 8.12 (s, 1H), 7.51-7.47 (m, 1H), 7.20-7.11 (m, 2H), 7.08-7.04 (m, 1H), 6.98 (s, 1H), 6.72 (dd, J=16.8, 10.7 Hz, 1H), 6.24 (d, J=2.9 Hz, 1H), 6.11 (dd, J=16.8, 2.0 Hz, 1H), 5.65 (dd, J=10.7, 2.0 Hz, 1H), 4.50-4.46 (m, 1H), 4.09-4.07 (m, 1H), 4.01 (s, 6H), 3.92 (s, 3H), 3.79-3.67 (m, 1H), 3.26-3.20 (m, 1H), 2.91-2.82 (m, 1H), 2.17-2.08 (m, 2H), 1.49-1.37 (m, 2H).

### Example 19: Preparation of 1-(4-((4-((2-fluoro-4-((2-(1-methyl-1H-pyrazol-4-yl)pyridin-4-yl)oxy)phenyl)amino)-7-methoxyquinazolin-6-yl)amino)piperidin-1-yl)prop-2-en-1-one (Compound 34)

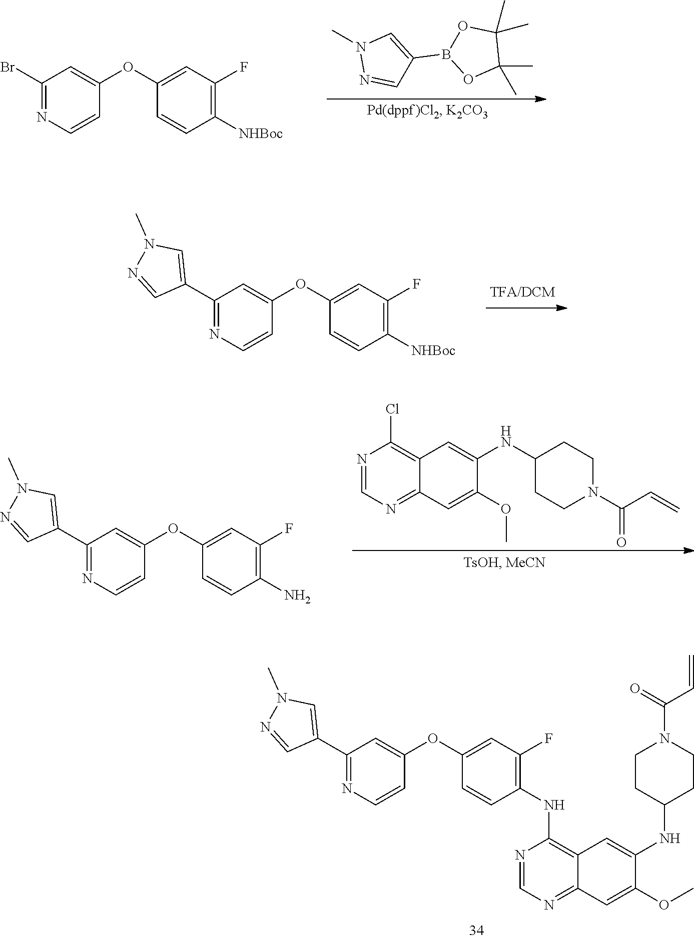

### Step 1: Preparation of tert-butyl (2-fluoro-4-((2-(1-methyl-1H-pyrazol-4-yl)pyridin-4-yl)oxy)phenyl)carbamate

To a solution of tert-butyl (4-((2-bromopyridin-4-yl)oxy)-2-fluorophenyl)carbamate (200 mg, 0.52 mmol) in 1,4-dioxane/H₂O (5:1)(6 mL) was add K₂CO₃ (78 mg, 0.57 mmol), 1-methyl-4-(4,4,5,5-tetramethyl-1,3,2-dioxaborolan-2-yl)-1H-pyrazole (144 mg, 1.04 mmol) and Pd(dppf)Cl₂ (38 mg, 0.052 mmol). The reaction mixture was stirred at 90° C. for 16 hours under N₂ atmosphere. The mixture was concentrated and purified by Combi-Flash (eluted by EA/PE=0-76%) to give tert-butyl (2-fluoro-4-((2-(1-methyl-1H-pyrazol-4-yl)pyridin-4-yl)oxy)phenyl)carbamate (150 mg, 75.1%) as a yellow oil. LC/MS: 384.9[M+H]⁺.

### Step 2: Preparation of 2-fluoro-4-((2-(1-methyl-1H-pyrazol-4-yl)pyridin-4-yl)oxy)aniline

A solution of tert-butyl (2-fluoro-4-((2-(1-methyl-1H-pyrazol-4-yl)pyridin-4-yl)oxy)phenyl)carbamate (150 mg, 0.39 mmol) in DCM/TFA=3:1 (4 mL) was stirred at 20° C. for 2 h. The mixture was diluted with DCM, washed with aq. NaHCO₃, concentrated and purified by Combi-Flash (EA/PE=0-60%) to afford the product (80 mg, 72.2%) as a yellow oil. LC/MS: 284.9 [M+H]⁺.

### Step 3: Preparation of 1-(4-((4-((2-fluoro-4-((2-(1-methyl-1H-pyrazol-4-yl)pyridin-4-yl)oxy)phenyl)amino)-7-methoxyquinazolin-6-yl)amino)piperidin-1-yl)prop-2-en-1-one (34)

To a mixture of 2-fluoro-4-{[2-(1-methylpyrazol-4-yl)pyridin-4-yl]oxy}aniline (40 mg, 0.14 mmol) and 1-{4-[(4-chloro-7-methoxyquinazolin-6-yl)amino]piperidin-1-yl}prop-2-en-1-one (49 mg, 0.14 mmol) in MeCN (2 mL) was added TsOH (2.4 mg, 0.014 mmol). The reaction was stirred at 75° C. for 2 h. The reaction was concentrated and purified by Prep-HPLC (using a gradient of 0.1% TFA/ACN from 15% to 30%) to give product (26 mg, 31.2%) as a yellow solid LC/MS: 594.8 [M+H]⁺. ¹H NMR (400 MHz, CD₃OD) δ8.60 (s, 1H), 8.56 (d, J=6.6 Hz, 1H), 8.43 (s, 1H), 8.17 (s, 1H), 7.79 (t, J=8.5 Hz, 1H), 7.70 (s, 1H), 7.49 (s, 1H), 7.42 (dd, J=10.3, 2.4 Hz, 1H), 7.31 (d, J=8.4 Hz, 1H), 7.24 (d, J=6.8 Hz, 1H), 7.19 (s, 1H), 6.85 (dd, J=16.8, 10.7 Hz, 1H), 6.24 (dd, J=16.8, 1.9 Hz, 1H), 5.79 (dd, J=10.6, 1.9 Hz, 1H), 4.63 (d, J=12.2 Hz, 1H), 4.24 (d, J=13.6 Hz, 1H), 4.15 (s, 3H), 4.02 (s, 3H), 3.93-3.84 (m, 1H), 3.41-3.37 (m, 1H), 3.04-3.01 (m, 1H), 2.26-2.23 (m, 2H), 1.67-1.53 (m, 2H).

### Example 20: 1-(4-((4-((2-fluoro-4-((2-morpholinopyridin-4-yl)oxy)phenyl)amino)-7-methoxyquinazolin-6-yl)amino)piperidin-1-yl)prop-2-en-1-one (Compound 35)

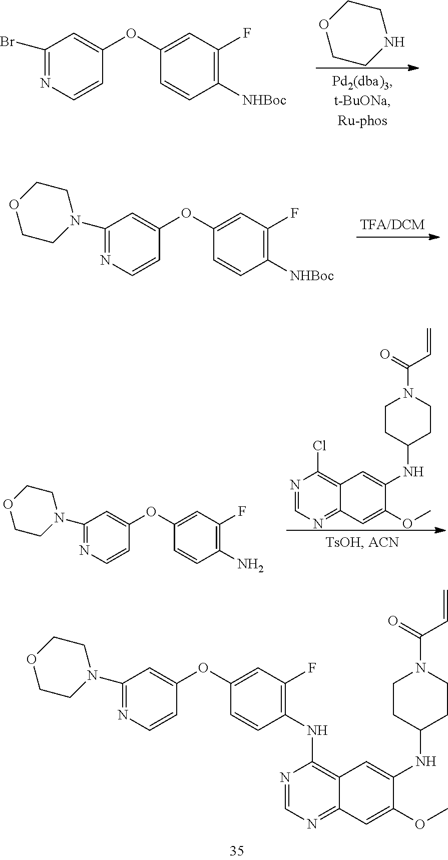 [SMILES: CC(C)(C)OC(=O)Nc1ccc(Oc2ccnc(Br)c2)cc1F ; C1COCCN1 ; CC(C)(C)OC(=O)Nc1ccc(Oc2ccnc(N3CCOCC3)c2)cc1F ; Nc1ccc(Oc2ccnc(N3CCOCC3)c2)cc1F ; C=CC(=O)N1CCC(Nc2cc3c(Cl)ncnc3cc2OC)CC1 ; C=CC(=O)N1CCC(Nc2cc3c(Nc4ccc(Oc5ccnc(N6CCOCC6)c5)cc4F)ncnc3cc2OC)CC1]

### Step 1: Preparation of tert-butyl (2-fluoro-4-((2-morpholinopyridin-4-yl)oxy)phenyl)carbamate

To a solution of tert-butyl (4-((2-bromopyridin-4-yl)oxy)-2-fluorophenyl)carbamate (200 mg, 0.52 mmol), morpholine (54 mg, 0.62 mmol) and sodium-t-butoxide (100 mg, 1.04 mmol) in toluene (12 mL) was added Ru-phos (49 mg, 0.104 mmol) and Pd₂(dba)₃ (48 mg, 0.052 mmol). The reaction mixture was stirred at 100° C. for 16 h. The reaction mixture was concentrated in vacuo. The residue was purified by column chromatography eluted with PE/EA=5:1 to afford tert-butyl (2-fluoro-4-((2-morpholinopyridin-4-yl)oxy)phenyl)carbamate (100 mg, 49.3%) as a yellow solid. LC/MS: 391.0 [M+H]⁺.

### Step 2: Preparation of 2-fluoro-4-((2-morpholinopyridin-4-yl)oxy)aniline

To a solution of tert-butyl (2-fluoro-4-((2-morpholinopyridin-4-yl)oxy)phenyl)carbamate (100 mg, 0.26 mmol) in DCM (1.5 mL) was added TFA (0.5 mL). The reaction mixture was stirred at 25° C. for 2 hr. The reaction mixture was concentrated in vacuo. The residue was purified by Combi-Flash (DCM/MeOH=10:1) to afford 2-fluoro-4-((2-morpholinopyridin-4-yl)oxy)aniline (37 mg, 49.9%) as a yellow solid. LC/MS: 291.0 [M+H]⁺.

### Step 3: Preparation of 1-(4-((4-((2-fluoro-4-((2-morpholinopyridin-4-yl)oxy)phenyl)amino)-7-methoxyquinazolin-6-yl)amino)piperidin-1-yl)prop-2-en-1-one (35)

To a solution 2-fluoro-4-{[2-(morpholin-4-yl)pyridin-4-yl]oxy}aniline (37 mg, 0.12 mmol) and 1-{4-[(4-chloro-7-methoxyquinazolin-6-yl)amino]piperidin-1-yl}prop-2-en-1-one (40 mg, 0.12 mmol) in ACN (2 mL) was added TsOH (2 mg, 0.012 mmol). The reaction mixture was stirred at 75° C. for 2 h. The reaction was concentrated and purified by Prep-HPLC, using a gradient of 0.1% NH₄OH/ACN from 80% to 60% to give 1-(4-((4-((2-fluoro-4-((2-morpholinopyridin-4-yl)oxy)phenyl)amino)-7-methoxyquinazolin-6-yl)amino)piperidin-1-yl)prop-2-en-1-one (7.3 mg, 10.1%) as a yellow solid. LC/MS: 600.0[M+H]⁺. ¹H NMR (400 MHz, CD₃OD) b 8.26 (d, J=5.5 Hz, 1H), 8.06 (d, J=5.7 Hz, 1H), 7.63 (t, J=8.7 Hz, 1H), 7.31 (s, 1H), 7.13-7.03 (m, 3H), 6.87-6.81 (m, 1H), 6.45 (d, J=6.3 Hz, 2H), 6.25-6.21 (m, 1H), 5.79-5.76 (m, 1H), 4.60 (d, J=13.8 Hz, 1H), 4.21 (d, J=14.2 Hz, 1H), 4.06 (s, 3H), 3.89-3.85 (m, 1H), 3.83-3.76 (m, 4H), 3.51-3.44 (m, 4H), 3.41-3.37 (m, 1H), 3.05-3.02 (m, 1H), 2.25-2.23 (m, 2H), 1.60-1.50 (m, 2H).

### Example 21: Preparation of 1-(4-((4-((2-fluoro-4-((2-(thiazol-4-yl)pyridin-4-yl)oxy)phenyl)amino)-7-methoxyquinazolin-6-yl)amino)piperidin-1-yl)prop-2-en-1-one (Compound 41)

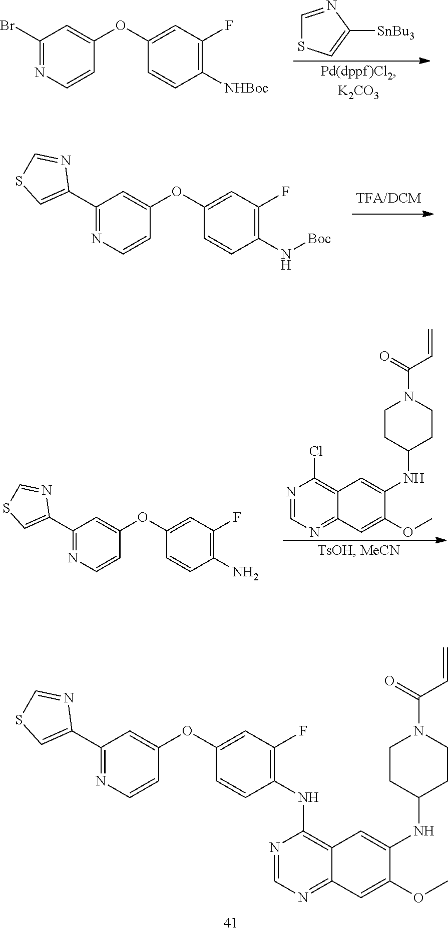

### Step 1: Preparation of tert-butyl (2-fluoro-4-((2-(thiazol-4-yl) pyridin-4-yl)oxy)phenyl)carbamate

A mixture of tert-butyl (4-((2-bromopyridin-4-yl)oxy)-2-fluorophenyl)carbamate (300 mg, 0.78 mmol), 4-(tributylstannyl)thiazole (291 mg, 0.78 mmol), Pd(dppf)Cl₂ (57 mg, 0.078 mmol) and K₂CO₃ (215 mg, 1.56 mmol) in dioxane (10 mL) was stirred at 100° C. for 15 h under nitrogen. The reaction mixture was concentrated and the residue was purified with Combi-Flash (PE/EA=0-40%) to give product (300 mg) as a yellow oil. LC/MS:388.1 [M+H]⁺.

### Step 2: Preparation of 2-fluoro-4-((2-(thiazol-4-yl)pyridin-4-yl)oxy)aniline

To a solution of tert-butyl (2-fluoro-4-((2-(thiazol-4-yl) pyridin-4-yl)oxy)phenyl)carbamate (300 mg, impure) in DCM (6 mL) was added TFA (2 mL) at 0° C. The mixture was stirred at 20° C. for 2 h. The resulting solution was poured into aq. NaHCO₃, extracted with DCM (50 mL×2). The combined organic layers were washed with brine, dried over Na₂SO₄, filtered and the filtrate was concentrated and purified by Combi-Flash (eluted by DCM/MeOH=0˜10%) to give 2-fluoro-4-{[2-(1,3-thiazol-4-yl)pyridin-4-yl]oxy}aniline (100 mg, 45% for two steps) as a yellow solid. LCMS: 287.9 [M+H]⁺

### Step 3: Preparation of 1-(4-((4-((2-fluoro-4-((2-(thiazol-4-yl)pyridin-4-yl)oxy)phenyl)amino)-7-methoxyquinazolin-6-yl)amino)piperidin-1-yl)prop-2-en-1-one (41)

To a mixture of 2-fluoro-4-{[2-(1,3-thiazol-4-yl)pyridin-4-yl]oxy}aniline (30 mg, 0.10 mmol) and 1-{4-[(4-chloro-7-methoxyquinazolin-6-yl)amino]piperidin-1-yl}prop-2-en-1-one (36 mg, 0.10 mmol) in MeCN (2 mL) was added TsOH (2 mg, 0.01 mmol). The reaction was stirred at 75° C. for 2 hr. The mixture was concentrated and purified by Prep-HPLC (using a gradient of 0.1% NH4OH/ACN from 25% to 50%) to give product (15 mg, 25%) as a yellow solid. LCMS: 598.0 [M+H]⁺. ¹HNMR (400 MHz, CD3OD) b 9.03 (d, J=1.9 Hz, 1H), 8.48 (d, J=5.7 Hz, 1H), 8.27-8.18 (m, 2H), 7.80 (d, J=2.4 Hz, 1H), 7.68-7.66 (m, 1H), 7.27 (s, 1H), 7.16 (dd, J=10.6, 2.5 Hz, 1H), 7.13-7.06 (m, 2H), 7.03 (dd, J=5.7, 2.4 Hz, 1H), 6.80 (dd, J=16.8, 10.7 Hz, 1H), 6.19 (dd, J=16.8, 1.9 Hz, 1H), 5.73 (dd, J=10.6, 1.9 Hz, 1H), 4.57-4.55 (m, 1H), 4.19-4.15 (m, 1H), 4.02 (s, 3H), 3.90-3.75 (m, 1H), 3.38-3.30 (m, 1H), 3.00-2.96 (m, 1H), 2.29-2.15 (m, 2H), 1.56-1.44 (m, 2H).

### Example 22: Preparation of 1-(4-((4-((2-fluoro-4-((4-(3-methyl-1,2,4-oxadiazol-5-yl)pyridin-2-yl)oxy)phenyl)amino)-7-methoxyquinazolin-6-yl)amino)piperidin-1-yl)prop-2-en-1-one (Compound 44)

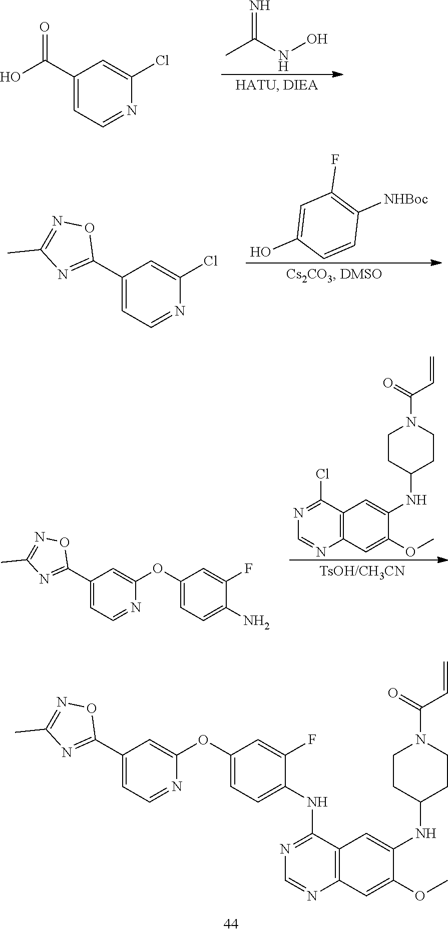

### Step 1: Preparation of 5-(2-chloropyridin-4-yl)-3-methyl-1,2,4-oxadiazole

A solution of 2-chloropyridine-4-carboxylic acid (1 g, 6.3 mmol) and HATU (2.9 g, 7.6 mmol) in DMF (50 mL) was stirred at 20° C. for 1 h. DIEA (2.5 g, 19 mmol) and N-hydroxyethanimidamide (517 mg, 6.9 mmol) was added to the mixture. The reaction mixture was stirred at 20° C. for 16 hr. The mixture was poured into ice water and extracted with EA (100 mL×3). The combined organic layers were dried over Na₂SO₄ and concentrated in vacuo. The residue was dissolved with Toluene (50 mL) and stirred at 110° C. for 16 hr. The mixture was concentrated and purified by Combi-Flash (PE/EA=0-50%) to give product (500 mg, 37%) as a yellow oil. LC/MS: 196.1[M+H]⁺.

### Step 2: Preparation of 2-fluoro-4-((4-(3-methyl-1,2,4-oxadiazol-5-yl) pyridin-2-yl) oxy) aniline

To a solution of 2-chloro-4-(3-methyl-1,2,4-oxadiazol-5-yl)pyridine (300 mg, 1.53 mmol) and tert-butyl N-(2-fluoro-4-hydroxyphenyl)carbamate (348 mg, 1.53 mmol) in DMSO (5 mL) was added Cs₂CO₃ (997 mg, 3.06 mmol). The mixture was stirred at 80° C. for 16 hr. LC-MS analysis indicated the Boc group was cleaved during reaction. The reaction mixture was poured into ice water and extracted with EA (20 mL×3). The combined organic layers were dried over Na₂SO₄, concentrated under vacuum and purified with Combi-Flash (PE/EA=1/1) to give product (100 mg, 23%) as a yellow solid. LC/MS: 287.1 [M+H]⁺.

### Step 3: Preparation of 1-(4-((4-((2-fluoro-4-((4-(3-methyl-1,2,4-oxadiazol-5-yl)pyridin-2-yl)oxy)phenyl)amino)-7-methoxyquinazolin-6-yl)amino)piperidin-1-yl)prop-2-en-1-one (44)

To a mixture of 2-fluoro-4-{[4-(3-methyl-1,2,4-oxadiazol-5-yl)pyridin-2-yl]oxy}aniline (40 mg, 0.14 mmol) and 1-{4-[(4-chloro-7-methoxyquinazolin-6-yl)amino]piperidin-1-yl}prop-2-en-1-one (48 mg, 0.14 mmol) in CH₃CN (2 mL) was added TsOH (2.4 mg, 0.014 mmol). The reaction was stirred for 2 h at 75° C. The mixture was concentrated and purified by Prep—HPLC (using a gradient of 0.1% FA/ACN from 30% to 50%) to give product (14.6 mg, 17.3%) as a yellow solid. LC/MS: 596.7[M+H]⁺. ¹H NMR (400 MHz, CD3OD) b 8.43 (d, J=5.2 Hz, 1H), 8.32 (s, 1H), 7.80 (d, J=5.2 Hz, 1H), 7.74-7.69 (m, 1H), 7.65-7.63 (m, 1H), 7.35 (s, 1H), 7.27-7.19 (m, 1H), 7.17-7.09 (m, 2H), 6.84 (dd, J=16.8, 10.7 Hz, 1H), 6.23 (dd, J=16.8, 2.0 Hz, 1H), 5.78 (dd, J=10.6, 2.0 Hz, 1H), 4.63-4.59 (m, 1H), 4.23-4.20 (m, 1H), 4.07 (s, 3H), 3.93-3.81 (m, 1H), 3.43-3.34 (m, 1H), 3.06-3.00 (m, 1H), 2.50 (s, 3H), 2.32-2.20 (m, 2H), 1.65-1.45 (m, 2H).

### Example 23: Preparation of 1-[4-({4-[(2-fluoro-4-{[4-(6-methylpyridin-3-yl)pyridin-2-yl]oxy}phenyl)amino]-7-methoxyquinazolin-6-yl}amino)piperidin-1-yl]prop-2-en-1-one (Compound 46)

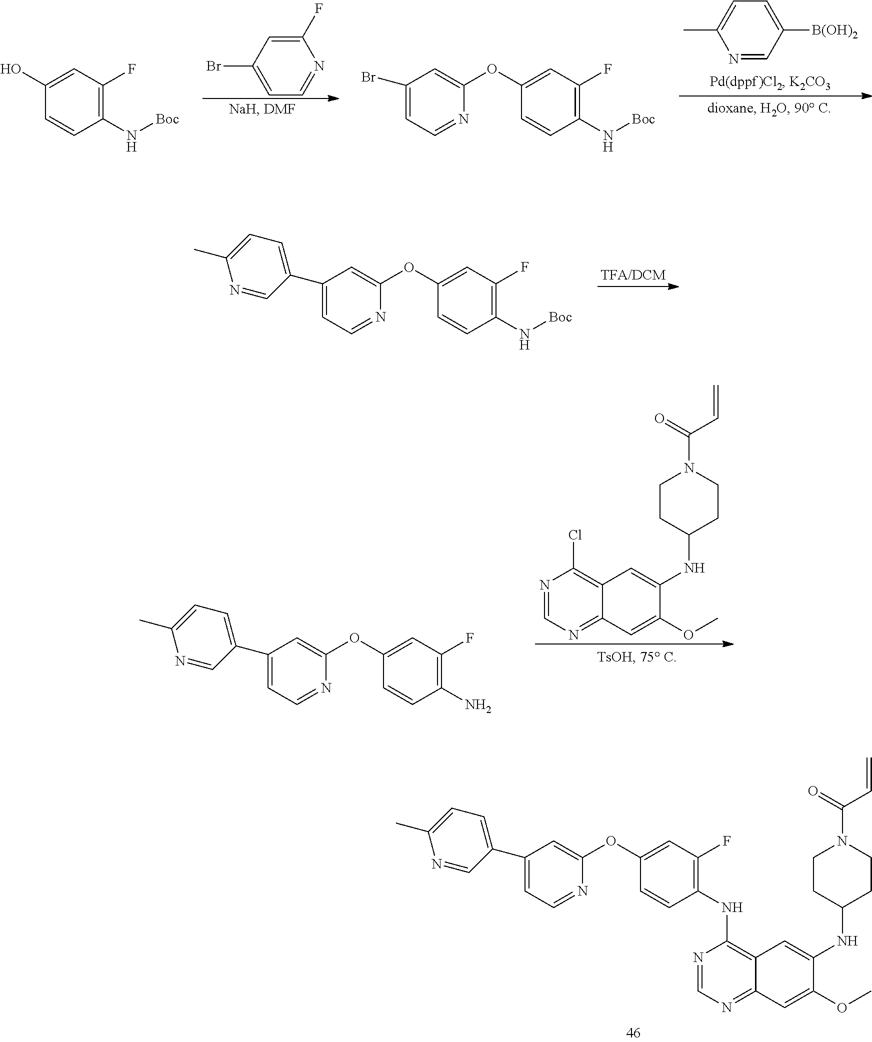

### Step 1: Preparation of tert-butyl (4-((4-bromopyridin-2-yl)oxy)-2-fluorophenyl)carbamate

To a solution of tert-butyl (2-fluoro-4-hydroxyphenyl)carbamate (300 mg, 1.31 mmol) in DMF (20 mL) was added NaH (60%, 105 mg, 2.62 mmol) at 0° C. The mixture was stirred at 0° C. for 30 min. 4-Bromo-2-fluoropyridine (243 mg, 1.38 mmol) was added. The mixture was stirred at 25° C. for 16 hr. The mixture was quenched with cold Sat. NH₄Cl and extracted with EA (20 mL). The organic layers were washed with brine, dried over Na₂SO₄, filtered and the filtrate was concentrated in vacuo and purified by combi-flash (0 to 10% gradient of DCM/CH₃OH) to afford the desired product (200 mg, 40%) as a white solid. LC/MS: 382.7 [M+H]⁺.

### Step 2: Preparation of tert-butyl (2-fluoro-4-((6-methyl-[3,4′-bipyridin]-2′-yl)oxy)phenyl)carbamate

To a solution of tert-butyl (4-((4-bromopyridin-2-yl)oxy)-2-fluorophenyl)carbamate (200 mg, 0.52 mmol) in 1,4-dioxane/H₂O (3:1) (4 ml) was added K₂CO₃ (144 mg, 1.04 mmol), (6-methylpyridin-3-yl)boronic acid (86 mg, 0.62 mmol) and Pd(dppf)Cl₂ (43 mg, 0.052 mmol). The reaction mixture was stirred at 90° C. for 16 hr under N₂ atmosphere.

The mixture was concentrated in vacuum and purified with Combi-Flash (PE/EA=0-25%) to give tert-butyl (2-fluoro-4-((6-methyl-[3,4′-bipyridin]-2′-yl)oxy)phenyl)carbamate (120 mg, 58.2%) as a white solid. LC/MS: 396.0[M+H]⁺.

### Step 3: Preparation of 2-fluoro-4-{[4-(6-methylpyridin-3-yl)pyridin-2-yl]oxy}aniline

A solution of tert-butyl (2-fluoro-4-((6-methyl-[3,4′-bipyridin]-2′-yl)oxy)phenyl)carbamate (120 mg, 0.30 mmol) in DCM/TFA 3:1 (4 mL) was stirred at r.t. for 2 h. The reaction was quenched with aq. NaHCO₃. The organic layers was concentrated in vacuo to give 2-fluoro-4-{[4-(6-methylpyridin-3-yl)pyridin-2-yl]oxy}aniline (40 mg, 44.8%) as a brown solid. LC/MS: 296.0[M+H]⁺.

### Step 4: Preparation of 1-[4-({4-[(2-fluoro-4-{[4-(6-methylpyridin-3-yl)pyridin-2-yl]oxy}phenyl)amino]-7-methoxyquinazolin-6-yl}amino)piperidin-1-yl]prop-2-en-1-one (46)

To a solution of 1-{4-[(4-chloro-7-methoxyquinazolin-6-yl)amino]piperidin-1-yl}prop-2-en-1-one (41 mg, 0.12 mmol) and 2-fluoro-4-{[4-(6-methylpyridin-3-yl)pyridin-2-yl]oxy}aniline (35 mg, 0.12 mmol) in CH₃CN (1.5 mL) was added TsOH (2.04 mg, 0.012 mmol).

The reaction was stirred for 2 h at 75° C. The reaction mixture was concentrated. The crude residue was purified by Prep-HPLC, using a gradient of 0.1% TFA/ACN from 70% to 10%, to give 1-[4-({4-[(2-fluoro-4-{[4-(6-methylpyridin-3-yl)pyridin-2-yl]oxy}phenyl)amino]-7-methoxyquinazolin-6-yl} amino)piperidin-1-yl]prop-2-en-1-one (39 mg, 54%) as a yellow solid. LC/MS: 605.8[M+H]⁺; ¹H NMR (400 MHz, CD3OD) b 9.03 (s, 1H), 8.56 (m, 2H), 8.31 (s, 1H), 7.82 (d, J=7.7 Hz, 1H), 7.54 (m, 4H), 7.17 (m, 3H), 6.80 (dd, J=16.0, 11.0 Hz, 1H), 6.20 (d, J=16.6 Hz, 1H), 5.74 (d, J=10.2 Hz, 1H), 4.62-4.58 (m, 1H), 4.22-4.18 (m, 1H), 4.10 (s, 3H), 3.84-3.81 (m, 1H), 3.35-3.31 (m, 1H), 2.99-2.96 (m, 1H), 2.75 (s, 3H), 2.21-2.17 (m, 2H), 1.55-1.51 (m, 2H).

### Example 24: 1-(4-((4-((4-((4-(2-cyclopropylpyrimidin-5-yl)thiazol-2-yl)oxy)-2-fluorophenyl)amino)-7-methoxyquinazolin-6-yl)amino)piperidin-1-yl)prop-2-en-1-one (Compound 51)

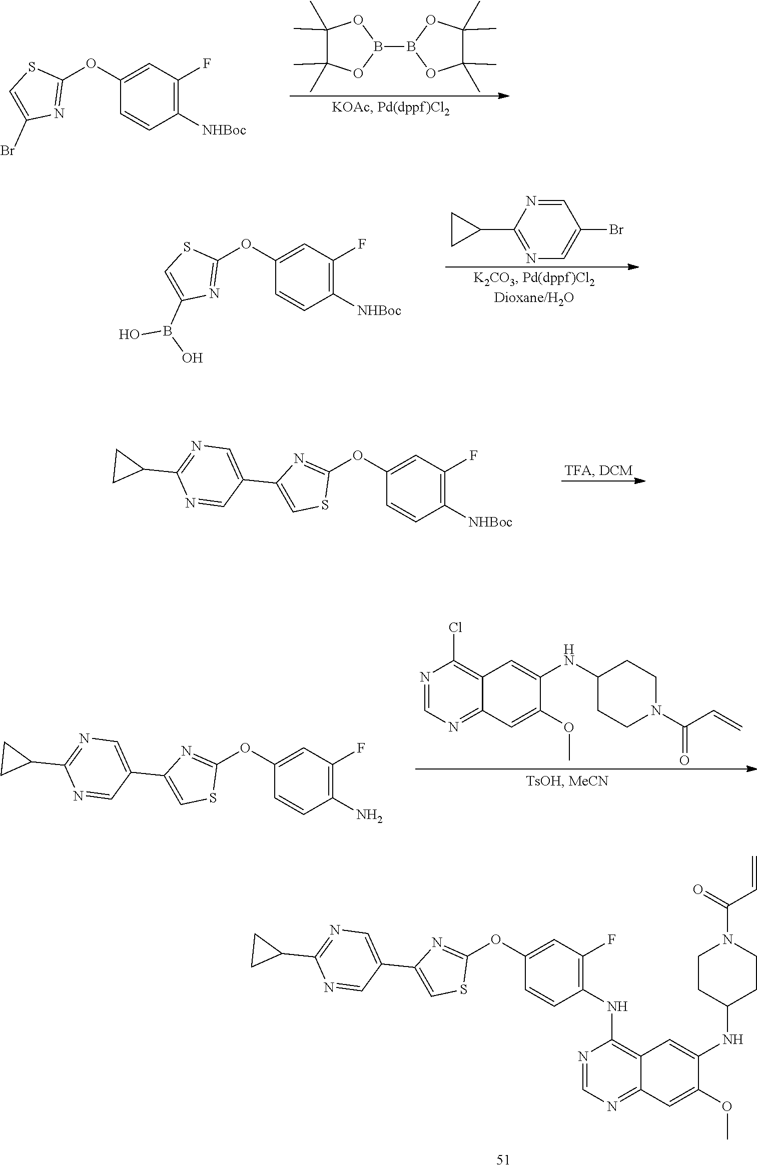

### Step 1: Preparation of (2-(4-((tert-butoxycarbonyl)amino)-3-fluorophenoxy)thiazol-4-yl)boronic acid

To a mixture of tert-butyl N-{4-[(4-bromo-1,3-thiazol-2-yl)oxy]-2-fluorophenyl}carbamate (600 mg, 1.5 mmol) and 4,4,5,5-tetramethyl-2-(4,4,5,5-tetramethyl-1,3,2-dioxaborolan-2-yl)-1,3,2-dioxaborolane (3.9 g, 15.4 mmol) in dioxane (15 mL) was added potassium acetate (302 mg, 3.0 mmol) and Pd(dppf)Cl₂ (113 mg, 0.15 mmol). The reaction was stirred at 90° C. for 20 hr under N₂. LC-MS analysis indicated the boronic acid was formed. The reaction mixture was concentrated. The crude residue was purified by Combi-Flash (eluted by EA/PE=1/1 to 2/1) to give boronic acid product (350 mg, 65.9%) as a white solid. LC/MS: 354.8[M+H]⁺.

### Step 2: Preparation of tert-butyl (4-((4-(2-cyclopropylpyrimidin-5-yl)thiazol-2-yl)oxy)-2-fluorophenyl)carbamate

To a mixture of (2-(4-((tert-butoxycarbonyl)amino)-3-fluorophenoxy)thiazol-4-yl)boronic acid (200 mg, 0.56 mmol) and 5-bromo-2-cyclopropylpyrimidine (124 mg, 0.62 mmol) in (1,4-dioxane/H₂O=5:1)(5 mL) was added Pd(dppf)Cl₂ (41.3 mg, 0.056 mmol) and K₂CO₃ (156.1 mg, 1.13 mmol) under N2. The reaction was stirred at 90° C. for 16 hr. The reaction mixture was concentrated and the crude residue was purified by Combi-Flash (eluted by PE/EA=5/1) to give the product (120 mg, 50%) as a yellow solid. LC/MS: 428.8[M+H]⁺.

### Step 3: Preparation of 4-((4-(2-cyclopropylpyrimidin-5-yl)thiazol-2-yl)oxy)-2-fluoroaniline

To a mixture of tert-butyl N-(4-{[4-(2-cyclopropylpyrimidin-5-yl)-1,3-thiazol-2-yl]oxy}-2-fluorophenyl)carbamate (160 mg, 0.28 mmol) in DCM (3 mL) was added TFA (1 mL). The reaction was stirred at 20° C. for 3 hr. The reaction mixture was diluted with DCM (10 mL). The organic layer was washed with sat. NaHCO₃ solution and brine, dried over Na₂SO₄, filtered and the filtrate was concentrated. The crude residue was purified by Combi-Flash (eluted by PE/EA=1/1) to give the product (60 mg, 65.3%) as a yellow solid. LC/MS: 328.8[M+H]⁺.

### Step 6: Preparation of 1-(4-((4-((4-((4-(2-cyclopropylpyrimidin-5-yl)thiazol-2-yl)oxy)-2-fluorophenyl)amino)-7-methoxyquinazolin-6-yl)amino)piperidin-1-yl)prop-2-en-1-one (51)

To a mixture of 4-{[4-(2-cyclopropylpyrimidin-5-yl)-1,3-thiazol-2-yl]oxy}-2-fluoroaniline (59 mg, 0.18 mmol) and 1-{4-[(4-chloro-7-methoxyquinazolin-6-yl)amino]piperidin-1-yl}prop-2-en-1-one (50 mg, 0.144 mmol) in MeCN (2 mL) was added TsOH (2.48 mg, 0.0144 mmol). The reaction was stirred at 75° C. for 2 hr. The reaction mixture was concentrated and purified by Prep-HPLC (using a gradient of 0.1% FA/ACN from 70% to 40%) to give product (30 mg, 32.5%) as a yellow solid. LC/MS: 638.7[M+H]⁺. ¹H NMR (400 MHz, CD₃OD) δ9.01 (s, 2H), 8.29 (s, 1H), 7.74-7.70 (m, 1H), 7.64 (s, 1H), 7.43 (dd, J=10.7, 2.6 Hz, 1H), 7.37-7.29 (m, 2H), 7.10 (s, 1H), 6.87-6.80 (m, 1H), 6.23 (dd, J=16.8, 1.9 Hz, 1H), 5.77 (dd, J=10.6, 1.9 Hz, 1H), 4.62-4.59 (m, 1H), 4.22-4.20 (m, 1H), 4.06 (s, 3H), 3.88-3.82 (m, 1H), 3.39-3.34 (m, 1H), 3.05-2.99 (m, 1H), 2.29-2.23 (m, 3H), 1.59-1.51 (m, 2H), 1.16-1.10 (m, 4H).

### Example 25: Preparation of 1-(6-((4-((2-fluoro-4-((1-(2-methoxypyrimidin-5-yl)-1H-pyrazol-3-yl) oxy) phenyl) amino)-7-methoxyquinazolin-6-yl) oxy)-2-azaspiro [3.3] heptan-2-yl) prop-2-en-1-one (Compound 79)

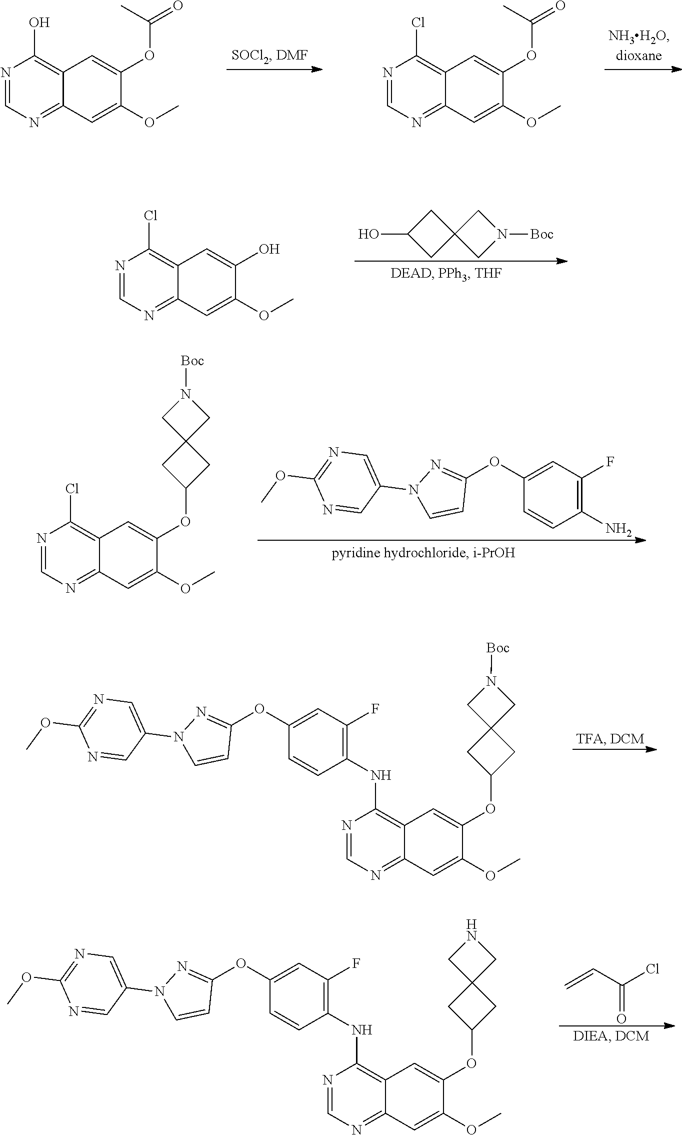

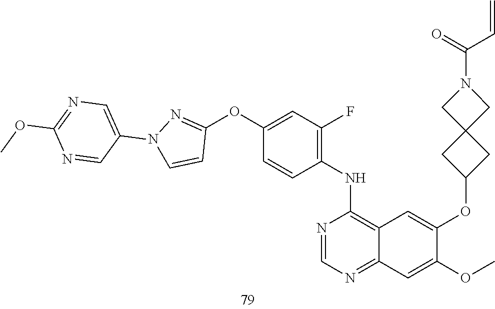

### Step 2: Preparation of 4-chloro-7-methoxyquinazolin-6-ol

To a solution of 4-chloro-7-methoxyquinazolin-6-yl acetate (300 mg, 1 0.19 mmol) in dioxane/H₂O (10 mL/2 mL) was added NH₃·H2O (0.5 mL) at 0° C. The reaction mixture was warmed to 25 00 and stirred at 25° C. for 2 hours. The reaction solution was concentrated to give product (290 mg, crude) as a light yellow solid which was used in the next step without further purification. LC/MS: 211 0.0 [M+H]⁺.

### Step 3: Preparation of tert-butyl 6-((4-chloro-7-methoxyquinazolin-6-yl) oxy)-2-azaspiro [3.3]heptane-2-carboxylate

To a solution of 4-chloro-7-methoxyquinazolin-6-ol (130 mg, 0.62 mmol) and tert-butyl 6-hydroxy-2-azaspiro [3.3] heptane-2-carboxylate (132 mg, 0.62 mmol) in THE (10 mL) was added PPh₃ (487 mg, 1 0.86 mmol) and DEAD (324 mg, 1 0.86 mmol) at 0° C. The reaction mixture was warmed to 50 00 and stirred for 1 hour. The reaction solution was concentrated, and the residue was purified with Combi-flash (PE/EtOAc=1:3) to give product (1 90 mg, 76%) as a yellow solid. LC/MS: 406.1 [M+H]⁺.

### Step 4: Preparation of tert-butyl 6-((4-((2-fluoro-4-((1-(2-methoxypyrimidin-5-yl)-1H-pyrazol-3-yl)oxy)phenyl)amino)-7-methoxyquinazolin-6-yl)oxy)-2-azaspiro[3.3]heptane-2-carboxylate

To a solution of tert-butyl 6-((4-chloro-7-methoxyquinazolin-6-yl)oxy)-2-azaspiro[3.3]heptane-2-carboxylate (130 mg, 0.33 mmol) and 2-fluoro-4-((1-(2-methoxypyrimidin-5-yl)-1H-pyrazol-3-yl)oxy)aniline (1 00 mg, 0.33 mmol) in i-PrOH (1 0 mL) was added pyridine hydrochloride (58 mg, 0.5 mmol) at 25° C. The reaction mixture was warmed to 80° C. and stirred for 2 hours. The reaction solution was concentrated, and the residue was purified with Combi-flash (DCM/MeOH=10:1) to give product (180 mg, 84%) as a light yellow solid. LC/MS: 671.2 [M+H]⁺.

### Step 5: Preparation of 6-((2-azaspiro[3.3]heptan-6-yl)oxy)-N-(2-fluoro-4-((1-(2-methoxypyrimidin-5-yl)-1H-pyrazol-3-yl)oxy)phenyl)-7-methoxyquinazolin-4-amine

A solution of tert-butyl 6-((4-((2-fluoro-4-((1-(2-methoxypyrimidin-5-yl)-1H-pyrazol-3-yl)oxy)phenyl)amino)-7-methoxyquinazolin-6-yl)oxy)-2-azaspiro[3.3]heptane-2-carboxylate (180 mg, 0.27 mmol) in DCM/TFA (6 mL/2 mL) was stirred at 25° C. for 1 hour. The reaction mixture was poured into water (50 mL) and extracted with DCM (50 mL×3), the organic layer was combined, dried over Na₂SO₄ and concentrated in vacuo to give product (50 mg, crude) as a yellow solid which was used in the next step without further purification. LC/MS: 571.1 [M+H]⁺.

### Step 6: Preparation of 1-(6-((4-((2-fluoro-4-((1-(2-methoxypyrimidin-5-yl)-1H-pyrazol-3-yl)oxy)phenyl)amino)-7-methoxyquinazolin-6-yl)oxy)-2-azaspiro[3.3]heptan-2-yl)prop-2-en-1-one (79)

To a solution of 6-((2-azaspiro[3.3]heptan-6-yl)oxy)-N-(2-fluoro-4-((1-(2-methoxypyrimidin-5-yl)-1H-pyrazol-3-yl)oxy)phenyl)-7-methoxyquinazolin-4-amine (45 mg, 0.08 mmol) and DIEA (21 mg, 0.16 mmol) in DCM (6 mL) was added acryloyl chloride (7.3 mg, 0.08 mmol) at −78° C. The solution mixture was stirred at −78° C. for 10 min and concentrated in vacuo. The residue was purified by Prep-HPLC (0.1% TFA in water/MeCN from 75% to 25%) to give the titled compound (7.9 mg, 16%) as a white solid. LC/MS: 625.2 [M+H]⁺. ¹H NMR (400 MHz, CD3OD) b 8.94 (s, 2H), 8.64 (s, 1H), 8.26 (d, J=2.7 Hz, 1H), 7.70 (s, 1H), 7.56 (t, J=8.8 Hz, 1H), 7.24-7.16 (m, 3H), 6.39-6.23 (m, 3H), 5.75-5.71 (m, 1H), 4.85-4.79 (m, 1H), 4.43-4.41 (m, 1H), 4.36-4.34 (m, 1H), 4.20-4.16 (m, 1H), 4.12-4.10 (m, 1H), 4.07 (s, 3H), 4.03 (s, 3H), 2.99-2.95 (m, 2H), 2.52-2.44 (m, 2H).

### Example 26: Preparation of (R)-1-(3-((4-((2-fluoro-4-((1-(2-methoxypyrimidin-5-yl)-1H-pyrazol-3-yl) oxy) phenyl) amino)-7-methoxyquinazolin-6-yl) oxy) pyrrolidin-1-yl) prop-2-en-1-one (Compound 80)

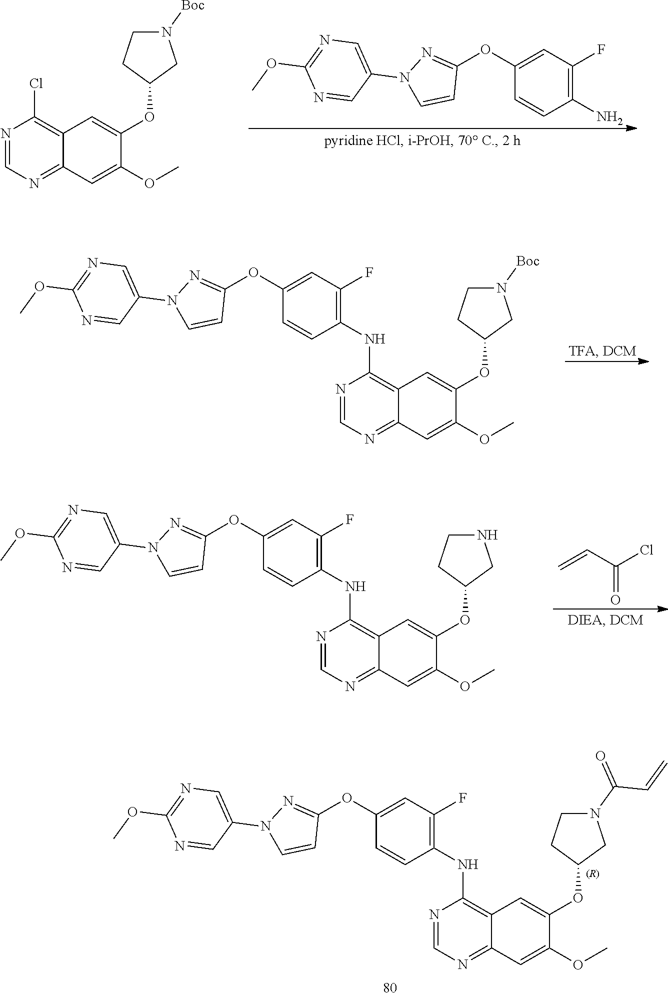

### Step 1: Preparation of tert-butyl (R)-3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidine-1-carboxylate

To a solution of tert-butyl (R)-3-((4-chloro-7-methoxyquinazolin-6-yl)oxy)pyrrolidine-1-carboxylate (150 mg, 0.4 mmol) and 2-fluoro-4-((1-(2-methoxypyrimidin-5-yl)-1H-pyrazol-3-yl)oxy)aniline (119 mg, 0.4 mmol) in i-PrOH (10 mL) was added pyridine hydrochloride (45 mg, 0.4 mmol). The reaction mixture was stirred at 70° C. for 2 hours. The reaction was concentrated and purified by Combi-Flash (EA/PE=3/7) to afford product (200 mg, 78%) as a yellow solid. LC/MS: 645.6[M+H]⁺.

### Step 2: Preparation of (R)—N-(2-fluoro-4-((1-(2-methoxypyrimidin-5-yl)-1H-pyrazol-3-yl)oxy)phenyl)-7-methoxy-6-(pyrrolidin-3-yloxy)quinazolin-4-amine

To a solution of tert-butyl (R)-3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidine-1-carboxylate (200 mg, 0.31 mmol) in DCM (12 mL) was added TFA (4 mL). The mixture was stirred at 20° C. for 30 min. The reaction mixture was poured into ice NaHCO₃ (10 mL) and extracted with DCM (10 mL×3). The organic layer was dried over with Na₂SO₄, concentrated in vacuo to afford product (100 mg, crude). LC/MS: 545.5 [M+H]⁺.

### Step 3: Preparation of (R)-1-(3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidin-1-yl)prop-2-en-1-one (80)

To a solution of tert-butyl (R)-3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidine-1-carboxylate (54 mg, 0.1 mmol) and DIEA (38 mg, 0.3 mmol) in DCM (10 mL) was added acryloyl chloride (7.2 mg, 0.08 mmol) at −78° C. After stirring at −78° C. for 10 min, the reaction mixture was concentrated and purified with Prep-HPLC (0.1% TFA in water/MeCN from 70% to 25%) to product (11 mg, 19%) as a yellow solid. LC/MS: 599.6[M+H]⁺. ¹H NMR (400 MHz, CD₃OD) b 8.95 (s, 2H), 8.34 (s, 1H), 8.28-8.23 (m, 2H), 7.80 (d, J=8.0 Hz, 1H), 7.58-7.54 (m, 1H), 7.20-7.13 (m, 2H), 6.71-6.57 (m, 2H), 6.33-6.25 (m, 2H), 5.80-5.74 (m, 1H), 5.29-5.23 (m, 1H), 4.05 (s, 3H), 4.01 (s, 3H), 3.95-3.78 (m, 5H), 2.44-2.35 (m, 2H).

### Example 27: Preparation of (S)-1-(3-((4-((2-fluoro-4-((1-(2-methoxypyrimidin-5-yl)-1H-pyrazol-3-yl) oxy) phenyl) amino)-7-methoxyquinazolin-6-yl) oxy) pyrrolidin-1-yl) prop-2-en-1-one (Compound 81)

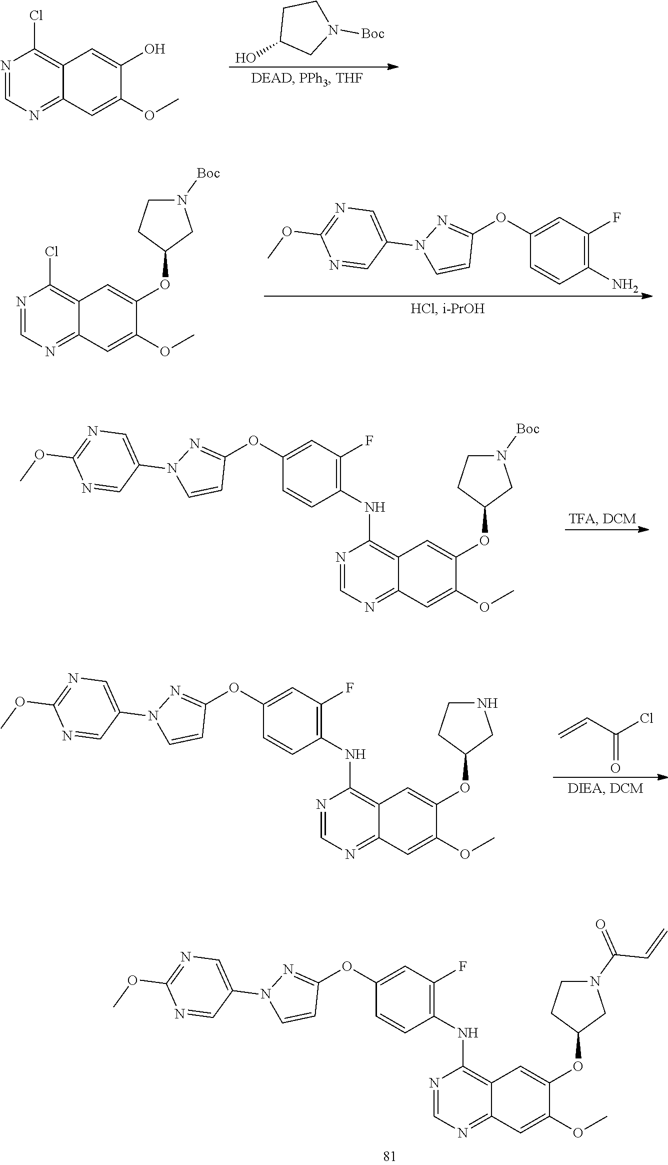

### Step 1: Preparation of tert-butyl (S)-3-((4-chloro-7-methoxyquinazolin-6-yl) oxy) pyrrolidine-1-carboxylate

To a solution of DEAD (249 mg, 1.43 mmol) in THE (10 ml) was added PPh₃ (800 mg, 2.86 mmol). The mixture was stirred for 0.5 h. 4-Chloro-7-methoxyquinazolin-6-ol (300 mg, 1.43 mmol) and tert-butyl (R)-3-hydroxypyrrolidine-1-carboxylate (267 mg, 1.43 mmol) was added into the mixture. The reaction was stirred at 50° C. for 3 h under N2 atmosphere. The mixture was concentrated and purified with Combi-flash (DCM/MeOH=10:1) to afford product (380 mg, 70%) as a light yellow solid. LC/MS: 380 [M+H]⁺.

### Step 2: Preparation of tert-butyl (S)-3-((4-((2-fluoro-4-((1-(2-methoxypyrimidin-5-yl)-1H-pyrazol-3-yl) oxy) phenyl) amino)-7-methoxyquinazolin-6-yl) oxy) pyrrolidine-1-carboxylate

To a solution of tert-butyl (S)-3-((4-chloro-7-methoxyquinazolin-6-yl)oxy)pyrrolidine-1-carboxylate (100 mg, 0.26 mmol) and 2-fluoro-4-((1-(2-methoxypyrimidin-5-yl)-1H-pyrazol-3-yl)oxy)aniline (78 mg, 0.26 mmol) in i-PrOH (15 mL) was added HCl/dioxane (0.1 mL). The reaction mixture was stirred at room temperature for 16 hours. The reaction mixture was concentrated and purified by Combi-flash (DCM/MeOH=10/1) to afford product (160 mg, 94%) as a light yellow solid. LC/MS: 645 [M+H]⁺.

### Step 3: Preparation of (S)—N-(2-fluoro-4-((1-(2-methoxypyrimidin-5-yl)-1H-pyrazol-3-yl) oxy) phenyl)-7-methoxy-6-(pyrrolidin-3-yloxy) quinazolin-4-amine

A solution of tert-butyl (S)-3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidine-1-carboxylate (120 mg, 0.19 mmol) in DCM/TFA (6 mL/2 mL) was stirred at room temperature for 2 hours. The reaction mixture was poured into water (50 mL) and extracted with DCM (50 mL×3). The organic layer was combined, dried over Na₂SO₄ and concentrated in vacuo to afford product (80 mg, crude) as a yellow solid. LC/MS: 545 [M+H]⁺.

### Step 4: Preparation of (S)-1-(3-((4-((2-fluoro-4-((1-(2-methoxypyrimidin-5-yl)-1H-pyrazol-3-yl) oxy) phenyl) amino)-7-methoxyquinazolin-6-yl) oxy) pyrrolidin-1-yl) prop-2-en-1-one (81)

To a solution of (S)—N-(2-fluoro-4-((1-(2-methoxypyrimidin-5-yl)-1H-pyrazol-3-yl)oxy)phenyl)-7-methoxy-6-(pyrrolidin-3-yloxy)quinazolin-4-amine (80 mg, 0.15 mmol) in DCM (10 mL) was added DIEA (38 mg, 0.3 mmol) and acryloyl chloride (13 mg, 0.15 mmol) at −78° C. The reaction mixture was stirred at −78° C. for 10 min. The reaction solution was concentrated and purified by Prep-HPLC (0.1% TFA in water/MeCN from 80% to 40%) to afford product (15 mg, 17%) as a white solid. LC/MS: 598 [M+H]⁺. ¹H NMR (400 MHz, CD₃OD) δ8.95 (s, 1H), 8.67 (s, 1H), 8.26 (d, J=8.4 Hz, 1H), 7.97 (s, 1H), 7.55-7.53 (m, 1H), 7.25 (s, 2H), 6.57-6.53 (m, 1H), 6.28-6.26 (m, 2H), 5.75-5.73 (m, 1H), 5.29-5.25 (m, 1H), 4.05 (s, 6H), 3.96-3.93 (m, 1H), 3.92-3.90 (m, 2H), 3.67-3.65 (m, 2H), 2.41-2.39 (m, 2H).

### Example 28: Preparation of 1-(6-((4-((2-fluoro-4-((1-(2-methoxypyrimidin-5-yl)-1H-pyrazol-3-yl)oxy)phenyl)amino)-7-methoxyquinazolin-6-yl)amino)-2-azaspiro[3.3]heptan-2-yl)prop-2-en-1-one (Compound 84)

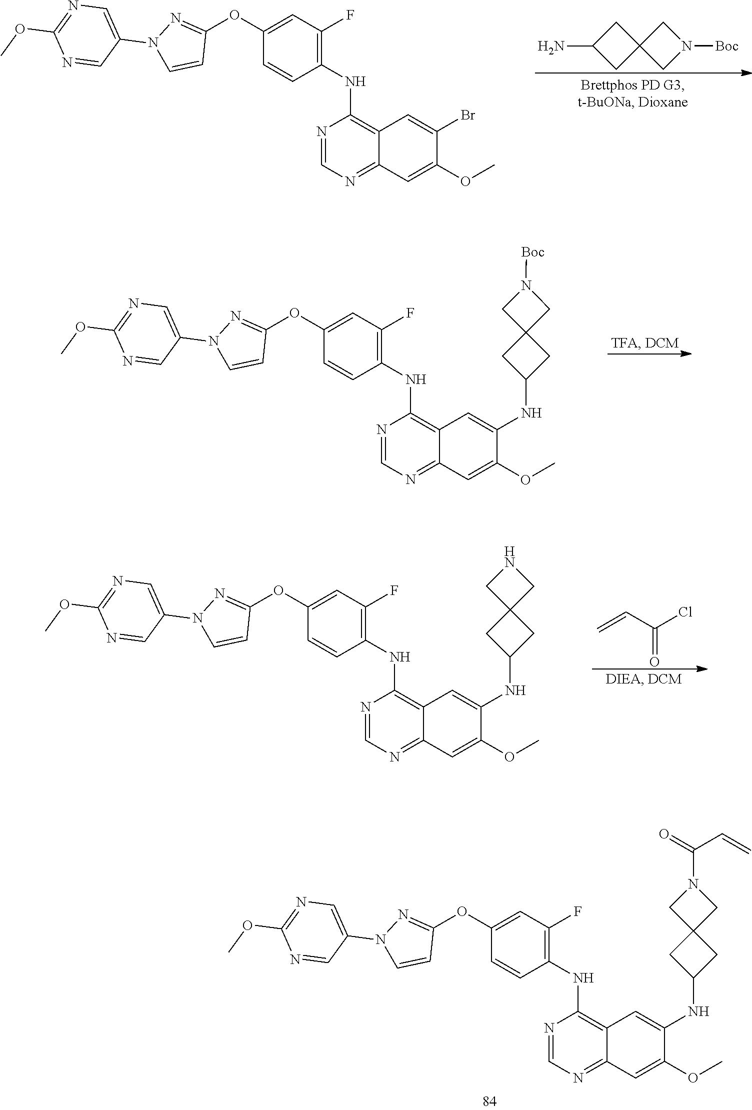

### Step 1: Preparation of tert-butyl 6-((4-((2-fluoro-4-((1-(2-methoxypyrimidin-5-yl)-1H-pyrazol-3-yl) oxy) phenyl) amino)-7-methoxyquinazolin-6-yl) amino)-2-azaspiro [3.3] heptane-2-carboxylate

To a solution of 6-bromo-N-(2-fluoro-4-((1-(2-methoxypyrimidin-5-yl)-1H-pyrazol-3-yl) oxy) phenyl)-7-methoxyquinazolin-4-amine (150 mg, 0.28 mmol) in dioxane (10 mL) was added tert-butyl 6-amino-2-azaspiro [3.3] heptane-2-carboxylate (59 mg, 0.28 mmol), Brettphos Pd G3 (25 mg, 0.028 mmol) and t-BuONa (54 mg, 0.56 mmol). The reaction mixture was stirred at 100° C. for 16 hours. The reaction solution was concentrated and purified with Combi-flash (DCM/MeOH=10:1) to give product (60 mg, 32%) as a light yellow solid. LC/MS: 670 [M+H]⁺.

### Step 2: Preparation of N4-(2-fluoro-4-((1-(2-methoxypyrimidin-5-yl)-1H-pyrazol-3-yl) oxy) phenyl)-7-methoxy-N6-(2-azaspiro [3.3] heptan-6-yl) quinazoline-4,6-diamine

A solution of tert-butyl 6-((4-((2-fluoro-4-((1-(2-methoxypyrimidin-5-yl)-1H-pyrazol-3-yl)oxy)phenyl)amino)-7-methoxyquinazolin-6-yl)amino)-2-azaspiro[3.3]heptane-2-carboxylate (55 mg, 0.08 mmol) in DCM/TFA (4.5 mL/1.5 mL) was stirred at room temperature for 1 hour. The reaction mixture was poured into water (10 mL) and extracted with DCM (20 mL×3). The organic layer was combined, dried over Na₂SO₄ and concentrated in vacuo to afford product (35 mg, crude) as a yellow solid. LC/MS: 570 [M+H]⁺.

### Step 3: Preparation of 1-(6-((4-((2-fluoro-4-((1-(2-methoxypyrimidin-5-yl)-1H-pyrazol-3-yl) oxy) phenyl) amino)-7-methoxyquinazolin-6-yl) amino)-2-azaspiro [3.3] heptan-2-yl) prop-2-en-1-one (84)

To a solution of N4-(2-fluoro-4-((1-(2-methoxypyrimidin-5-yl)-1H-pyrazol-3-yl)oxy)phenyl)-7-methoxy-N6-(2-azaspiro[3.3]heptan-6-yl)quinazoline-4,6-diamine (30 mg, 0.05 mmol) in DCM (6 mL) was added DIEA (14 mg, 0.10 mmol) and acryloyl chloride (4.8 mg, 0.05 mmol) at −78° C. The solution was stirred for 10 min at −78° C. The reaction mixture solution was concentrated and purified by Prep-HPLC (0.1% TFA in water/MeCN from 80% to 30%) to afford product (6.9 mg, 21%) as a white solid. LC/MS: 624 [M+H]⁺. ¹H NMR (400 MHz, CD3OD) b 8.94 (s, 2H), 8.51 (s, 1H), 8.26 (d, J=2.4 Hz, 1H), 7.55 (t, J=8.4 Hz, 1H), 7.24-7.14 (m, 3H), 7.12 (s, 1H), 6.36-6.19 (m, 3H), 5.77-5.69 (m, 1H), 4.45-4.41 (m, 1H), 4.30-4.26 (m, 1H), 4.21-4.17 (m, 1H), 4.12 (s, 3H), 4.05-4.04 (m, 5H), 2.92-2.83 (m, 2H), 2.32-2.24 (m, 2H).

### Example 29: Preparation of 1-(4-((4-((2-fluoro-4-((2-(2-methylthiazol-4-yl) pyridin-4-yl) oxy) phenyl) amino)-7-meth oxyquinazolin-6-yl) amino) piperidin-1-yl) prop-2-en-1-one (Compound 97)

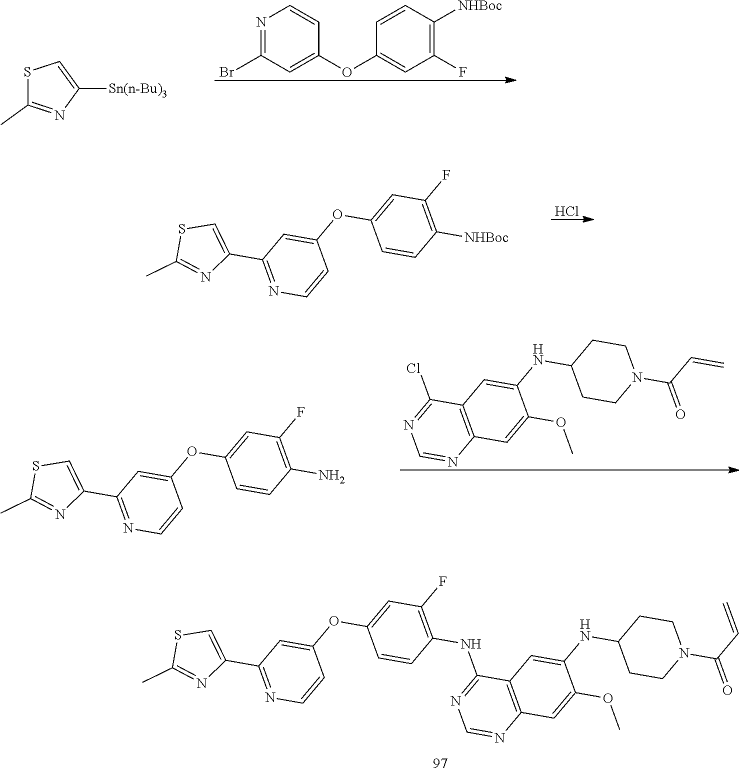

### Step 1: Preparation of tert-butyl (2-fluoro-4-((2-(2-methylthiazol-4-yl) pyridin-4-yl) oxy) phenyl) carbamate

To a solution of 2-methyl-4-(tributylstannyl)thiazole (810.4 mg, 2.08 mmol) in THE (20 mL) stirred at room temperature was added tert-butyl (4-((2-bromopyridin-4-yl) oxy)-2-fluorophenyl) carbamate (400 mg, 1.04 mmol) and PdCl₂(PPh₃)₂ (73.3 mg, 0.10 mmol). The reaction mixture was stirred at 75° C. overnight. The solvent was removed in vacuum and the residue was washed with DCM (150 mL×3). The solution was washed with brine and dried over Na₂SO₄. The solvent was removed in vacuum to give a crude product. The crude product was purified by silica-gel column chromatography and eluted with 0-30% EA in PE to give the title compound (200 mg, 47.7%) as a yellow oil. LC/MS: 402.1 [M+H]⁺.

### Step 2: Preparation of 2-fluoro-4-((2-(2-methylthiazol-4-yl) pyridin-4-yl) oxy) aniline

A solution of tert-butyl (2-fluoro-4-((2-(2-methylthiazol-4-yl) pyridin-4-yl) oxy) phenyl) carbamate (200 mg, 0.49 mmol) in HCl/dioxane (4 N, 50 mL) was stirred at room temperature for 2 hours. The solvent was removed in vacuum to give a crude product. The crude product was purified by silica-gel column chromatography and eluted with 0-10% MeOH in DCM to give the title compound (130 mg, 88.0%) as a yellow solid. LC/MS: 302.0 [M+H]⁺.

### Step 3: Preparation of 1-(4-((4-((2-fluoro-4-((2-(2-methylthiazol-4-yl) pyridin-4-yl) oxy) phenyl) amino)-7-methoxyquinazolin-6-yl) amino) piperidin-1-yl) prop-2-en-1-one (97)

To a solution of 2-fluoro-4-((2-(2-methylthiazol-4-yl) pyridin-4-yl) oxy) aniline (80 mg, 0.26 mmol) in ACN (30 mL) stirred under nitrogen at room temperature was added 1-{4-[(4-chloro-7-methoxyquinazolin-6-yl) amino] piperidin-1-yl} prop-2-en-1-one (92 mg, 0.26 mmol) and TsOH (137.2 mg, 0.79 mmol). The reaction mixture was stirred at 80° C. for 2 hours. After evaporation of solvent, the crude product was purified by silica-gel column chromatography and eluted with 0-10% MeOH in DCM to give the title compound (18.6 mg, 11.6%) as a yellow solid. LC/MS: 612.2 [M+H]⁺. ¹H NMR (400 MHz, DMSO-d6) δ=9.28 (s, 1H), 8.56 (d, J=5.2 Hz, 1H), 8.42-8.03 (m, 4H), 7.66 (t, J=8.2 Hz, 1H), 7.54 (s, 1H), 7.38 (d, J=9.8 Hz, 1H), 7.29 (s, 1H), 7.16 (d, J=7.6 Hz, 1H), 7.12-7.01 (m, 2H), 6.87 (dd, J=16.3, 10.2 Hz, 1H), 6.12 (d, J=17.0 Hz, 1H), 5.69 (d, J=10.2 Hz, 1H), 5.36 (d, J=8.2 Hz, 1H), 4.54-4.43 (m, 1H), 4.20-4.09 (m, 1H), 3.97 (s, 3H), 3.80-3.73 (m, 1H), 3.26-3.20 (m, 1H), 2.90-2.82 (m, 1H), 2.71 (s, 3H), 2.12-2.06 (m, 2H), 1.52-1.36 (m, 2H).

### Example 30: Preparation of 1-(4-((4-((2-fluoro-4-((2-(piperidin-1-yl)pyridin-4-yl)oxy)phenyl)amino)-7-methoxyquinazolin-6-yl)amino)piperidin-1-yl)prop-2-en-1-one (Compound 100)

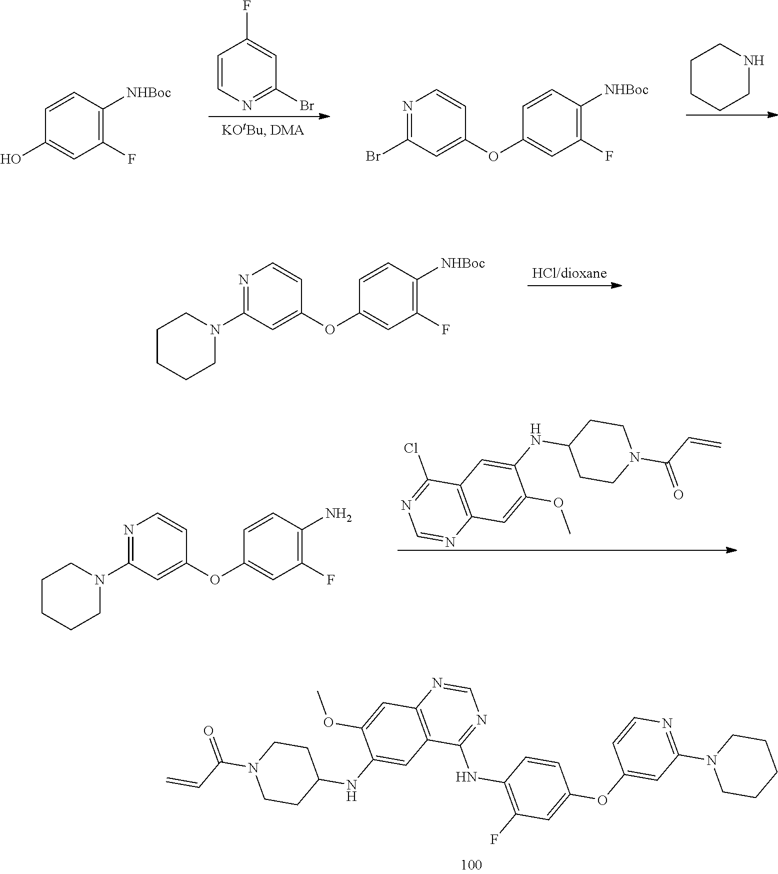

### Step 1: Preparation of tert-butyl (4-((2-bromopyridin-4-yl)oxy)-2-fluorophenyl)carbamate

To a solution of tert-butyl (2-fluoro-4-hydroxyphenyl)carbamate (1.4 g, 6.17 mmol) in DMA (10 mL) was added t-BuOK (0.83 g, 7.41 mmol). The mixture was stirred at room temperature for 5 minutes followed by addition of 2-bromo-4-fluoropyridine (1.2 g, 6.86 mmol). The reaction was stirred at 70° C. for 12 hours under Ar. The mixture was diluted with H₂O (10 mL) and extracted with EtOAc (10 mL×3). The combined organic layer was washed with brine, dried over Na₂SO₄, and concentrated in vacuum to give a crude product. The crude product was purified by flash chromatography with PE:EA=4:1 to give the desired product (2.34 g, 95% purity, 93.5% yield). LC/MS: 382.8 [M+H]⁺.

### Step 2: Preparation of tert-butyl (2-fluoro-4-((2-(piperidin-1-yl)pyridin-4-yl)oxy)phenyl)carbamate

The mixture of tert-butyl (4-((2-bromopyridin-4-yl)oxy)-2-fluorophenyl)carbamate (60 mg, 0.16 mmol) and piperidine (2 mL) was stirred at 100° C. for 17 hours. The solvent was removed in vacuum and the residue was purified by Flash Chromatography with PE:EA=1:1 to give the desired product (50 mg, 95% purity, 76.7% yield). LC/MS: 388.0 [M+H]⁺.

### Step 3: Preparation of 2-fluoro-4-((2-(piperidin-1-yl)pyridin-4-yl)oxy)aniline

A solution of tert-butyl (2-fluoro-4-((2-(piperidin-1-yl)pyridin-4-yl)oxy)phenyl)carbamate (50 mg, 0.13 mmol) in HCl/dioxane (4 N, 2 mL) was stirred at room temperature for 2 hours. The solvent was removed in vacuum to give the crude product which was used in the next step without further purification (50 mg). LC/MS: 288.1 [M+H]⁺.

### Step 4: Preparation of 1-(4-((4-((2-fluoro-4-((2-(piperidin-1-yl)pyridin-4-yl)oxy)phenyl)amino)-7-methoxyquinazolin-6-yl)amino)piperidin-1-yl)prop-2-en-1-one (100)

To a solution of 2-fluoro-4-((2-(piperidin-1-yl)pyridin-4-yl)oxy)aniline (50 mg, crude) in MeCN (5 mL) was added 1-(4-((4-chloro-7-methoxyquinazolin-6-yl)amino)piperidin-1-yl)prop-2-en-1-one (66.9 mg, 0.19 mmol) and TsOH (2.7 mg, 0.015 mmol). The reaction was stirred at 80° C. for 2 hours. The mixture was basified with aq. NaHCO₃ solution and extracted with DCM:MeOH=10:1 (10 mL×3). The combined organic layer was washed with brine, dried over Na₂SO₄ and concentrated in vacuum to give a crude product. The crude product was purified by Prep-TLC with DCM:MeOH=10:1 to give the desired product (16.7 mg, 21.2% yield for two steps). LC/MS: 598.3 [M+H]⁺. ¹H NMR (400 MHz, DMSO-d6) δ 9.66 (brs, 1H), 8.33 (s, 1H), 8.05 (d, J=5.7 Hz, 1H), 7.58 (t, J=8.8 Hz, 1H), 7.37 (s, 1H), 7.24-7.21 (m, 1H), 7.11 (s, 1H), 7.06-7.03 (m, 1H), 6.86 (dd, J=16.7, 10.5 Hz, 1H), 6.40 (d, J=2.0 Hz, 1H), 6.21 (dd, J=5.7, 2.0 Hz, 1H), 6.12 (dd, J=16.7, 2.5 Hz, 1H), 5.69 (dd, J=10.4, 2.5 Hz, 1H), 5.48 (d, J=8.5 Hz, 1H), 4.46 (d, J=13.0 Hz, 1H), 4.13 (d, J=13.6 Hz, 1H), 3.98 (s, 3H), 3.85-3.72 (m, 1H), 3.50-3.47 (m, 4H), 2.86 (t, J=11.7 Hz, 1H), 2.12-2.02 (m, 2H), 1.64-1.37 (m, 9H).

### Example 31: Preparation of 1-(4-((4-((4-((2-((2R,6S)-2,6-dimethylmorpholino)pyridin-4-yl)oxy)-2-fluorophenyl)amino)-7-methoxyquinazolin-6-yl)amino)piperidin-1-yl)prop-2-en-1-one (101)

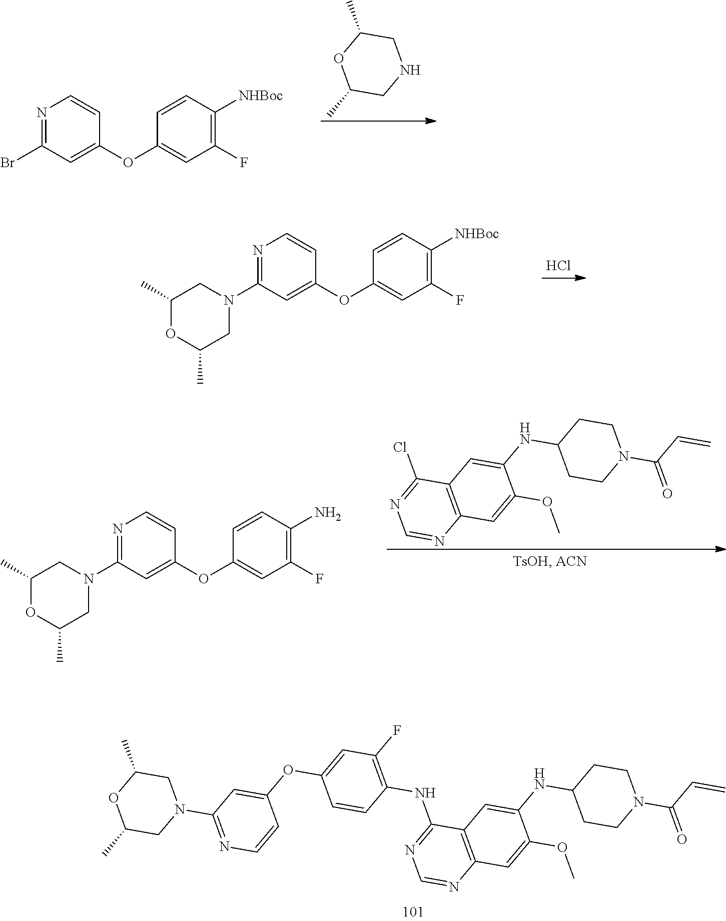

### Step 1: Preparation of tert-butyl (4-((2-((2R,6S)-2,6-dimethylmorpholino)pyridin-4-yl)oxy)-2-fluorophenyl)carbamate

The mixture of tert-butyl N-{4-[(2-bromopyridin-4-yl)oxy]-2-fluorophenyl}carbamate (700 mg, 0.78 mmol) in (2R,6S)-2,6-dimethylmorpholine (20 mL) was stirred at 80° C. for overnight. The reaction mixture was concentrated in vacuum and the residue was dissolved with DCM (150 mL). The organic phase was washed with brine and dried over Na₂SO₄. The solvent was removed in vacuum to give a crude product. The crude product was purified by silica-gel column chromatography and eluted with 0-30% EA in PE to give the title compound (600 mg, 58.1%) as a white solid. LC/MS: 418.2 [M+H]⁺.

### Step 2: Preparation of 4-((2-((2R,6S)-2,6-dimethylmorpholino)pyridin-4-yl)oxy)-2-fluoroaniline

A solution of tert-butyl N-[4-({2-[(2R,6S)-2,6-dimethylmorpholin-4-yl]pyridin-4-yl}oxy)-2-fluorophenyl]carbamate (600 mg, 1.43 mmol) in HCl/dioxane (4 N, 50 mL). was stirred at room temperature for 2 hours. The solvent was removed in vacuum and the residue was dissolved with DCM (150 mL). The organic phase was washed with brine and dried over Na₂SO₄. The crude product was purified by silica-gel column chromatography and eluted with 0-10% MeOH in DCM to give the title compound (450 mg, 98.6%) as a white solid. LC/MS: 318.1 [M+H]⁺.

### Step 3: Preparation of 1-(4-((4-((4-((2-((2R,6S)-2,6-dimethylmorpholino)pyridin-4-yl)oxy)-2-fluorophenyl)amino)-7-methoxyquinazolin-6-yl)amino)piperidin-1-yl)prop-2-en-1-one (101)

To a solution of 4-({2-[(2R,6S)-2,6-dimethylmorpholin-4-yl]pyridin-4-yl}oxy)-2-fluoroaniline (250 mg, 0.78 mmol) in ACN (30 mL) stirred under argon was added 1-{4-[(4-chloro-7-methoxyquinazolin-6-yl)amino]piperidin-1-yl}prop-2-en-1-one (328 mg, 0.94 mmol) and TsOH (543 mg, 3.15 mmol) at room temperature. The reaction mixture was stirred at 80° C. for 2 hours. After evaporation of solvent, the crude product was purified by silica-gel column chromatography and eluted with 0-10% MeOH in DCM to give the title compound (140 mg, 27.4% yield) as a yellow solid. LC/MS:628.2 [M+H]⁺. ¹H NMR (400 MHz, DMSO) b 9.25 (s, 1H), 8.24 (s, 1H), 8.07 (d, J=5.6 Hz, 1H), 7.57 (t, J=8.8 Hz, 1H), 7.31-7.16 (m, 2H), 7.12-6.99 (m, 2H), 6.86 (dd, J=16.8, 10.5 Hz, 1H), 6.50 (d, J=2.0 Hz, 1H), 6.26 (dd, J=5.8, 2.0 Hz, 1H), 6.12 (dd, J=16.8, 2.5 Hz, 1H), 5.69 (dd, J=10.4, 2.5 Hz, 1H), 5.36 (d, J=8.4 Hz, 1H), 4.46 (d, J=12.8 Hz, 1H), 4.22-4.06 (m, 3H), 3.97 (s, 3H), 3.81-3.72 (m, 1H), 3.65-3.52 (m, 2H), 3.19 (t, J=12.8 Hz, 1H), 2.83 (t, J=11.6 Hz, 1H), 2.39 (dd, J=12.8, 10.7 Hz, 2H), 2.15-2.01 (m, 2H), 1.53-1.37 (m, 2H), 1.18 (d, J=5.6 Hz, 6H).

### Example 32: Preparation of 1-(4-((4-((2-fluoro-4-((2-(3-(trifluoromethyl)pyrrolidin-1-yl)pyridin-4-yl)oxy) phenyl) amino)-7-methoxyquinazolin-6-yl)amino) piperidin-1-yl) prop-2-en-1-one (Compound 150)

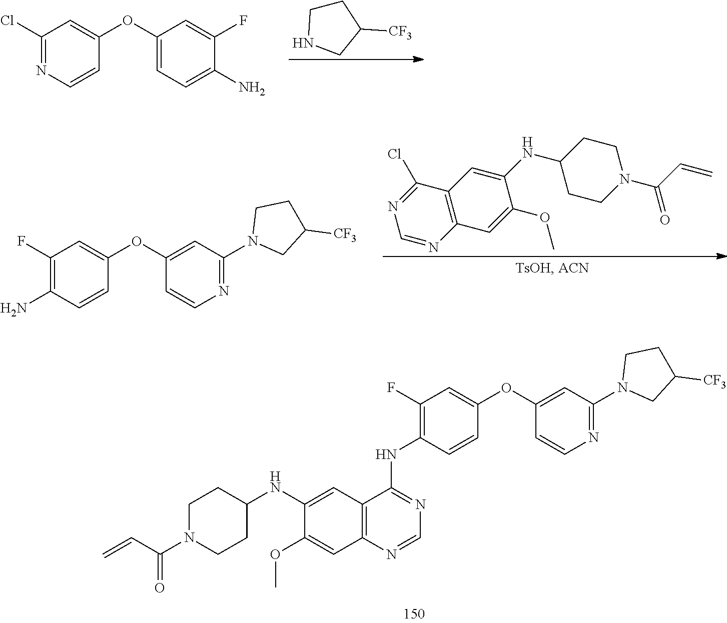 [SMILES: Nc1ccc(Oc2ccnc(Cl)c2)cc1F ; FC(F)(F)C1CCNC1 ; Nc1ccc(Oc2ccnc(N3CCC(C(F)(F)F)C3)c2)cc1F ; C=CC(=O)N1CCC(Nc2cc3c(Cl)ncnc3cc2OC)CC1 ; C=CC(=O)N1CCC(Nc2cc3c(Nc4ccc(Oc5ccnc(N6CCC(C(F)(F)F)C6)c5)cc4F)ncnc3cc2OC)CC1]

### Step 1: Preparation of 2-fluoro-4-((2-(3-(trifluoromethyl)pyrrolidin-1-yl)pyridin-4-yl)oxy)aniline

To a solution of 4-((2-chloropyridin-4-yl)oxy)-2-fluoroaniline (200 mg, 0.84 mmol) in DMSO (5 mL) stirred at room temperature was added 3-(trifluoromethyl)pyrrolidine (587.2 mg, 4.19 mmol) and Cs₂CO₃ (1.64 g, 5.03 mmol). The reaction mixture was stirred at 120° C. for 16 hours. The mixture was partitioned between EA and H₂O. The organic phase was washed with brine, dried over Na₂SO₄, and concentrated in vacuum to give a crude product.

The crude product was purified by Pre-TLC (DCM:MeOH=10:1) to give the desired product (70 mg, 24.4%) as a yellow solid. LC/MS: 342.1 [M+H]⁺.

### Step 2: Preparation of 1-(4-((4-((2-fluoro-4-((2-(3-(trifluoromethyl)pyrrolidin-1-yl)pyridin-4-yl)oxy)phenyl)amino)-7-methoxyquinazolin-6-yl)amino)piperidin-1-yl)prop-2-en-1-one (150)

To a solution of 2-fluoro-4-((2-(3-(trifluoromethyl)pyrrolidin-1-yl)pyridin-4-yl)oxy)aniline (65 mg, 0.19 mmol) in MeCN (5 mL) stirred at room temperature was added 1-(4-((4-chloro-7-methoxyquinazolin-6-yl)amino)piperidin-1-yl)prop-2-en-1-one (72.5 mg, 0.21 mmol) and TsOH (98 mg, 0.57 mmol). The reaction mixture was stirred at 80° C. for 2 hours. The mixture was basified with Et3N and concentrated under vacuum to give a crude product. The crude product was purified by Prep-HPLC to give the formate salt of the titled product (20 mg, 16.1%) as a yellow solid. LC/MS: 652.3 [M+H]⁺. ¹H NMR (400 MHz, DMSO-d6) b 9.24 (brs, 1H), 8.24 (s, 1H), 8.05 (d, J=5.7 Hz, 1H), 7.58 (t, J=8.8 Hz, 1H), 7.26 (s, 1H), 7.22 (dd, J=11.0, 2.6 Hz, 1H), 7.08 (s, 1H), 7.04 (dd, J=8.6, 1.8 Hz, 1H), 6.86 (dd, J=16.7, 10.5 Hz, 1H), 6.26 (dd, J=5.7, 2.1 Hz, 1H), 6.14 (d, J=2.5 Hz, 0.5H), 6.11 (d, J=2.1 Hz, 1H), 6.10 (d, J=2.5 Hz, 0.5H), 5.69 (dd, J=10.4, 2.5 Hz, 1H), 5.36 (d, J=8.4 Hz, 1H), 4.46 (d, J=13.3 Hz, 1H), 4.13 (d, J=11.5 Hz, 1H), 3.97 (s, 3H), 3.74-3.66 (m, 2H), 3.50-3.37 (m, 3H), 3.25-3.19 (m, 1H), 2.86-2.83 (m, 1H), 2.33-2.20 (m, 2H), 2.14-2.02 (m, 3H), 1.47-1.41 (m, 2H).

### Example 33: Preparation of 1-(4-(4-((6-((1-acryloylpiperidin-4-yl)amino)-7-methoxyquinazolin-4-yl)amino)-3-fluorophenoxy)pyridin-2-yl)pyrrolidine-3-carbonitrile (Compound 151)

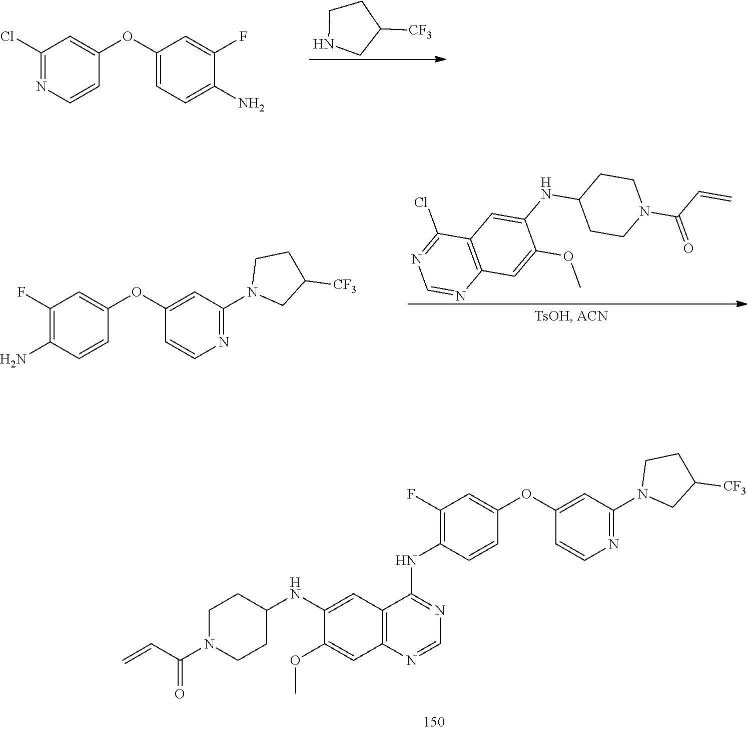 [SMILES: Nc1ccc(Oc2ccnc(Cl)c2)cc1F ; FC(F)(F)C1CCNC1 ; Nc1ccc(Oc2ccnc(N3CCC(C(F)(F)F)C3)c2)cc1F ; C=CC(=O)N1CCC(Nc2cc3c(Cl)ncnc3cc2OC)CC1 ; C=CC(=O)N1CCC(Nc2cc3c(Nc4ccc(Oc5ccnc(N6CCC(C(F)(F)F)C6)c5)cc4F)ncnc3cc2OC)CC1]

### Step 1: Preparation of tert-butyl (4-((2-(3-cyanopyrrolidin-1-yl)pyridin-4-yl)oxy)-2-fluorophenyl)carbamate

The mixture of pyrrolidine-3-carbonitrile hydrochloride (173 mg, 1.30 mmol), tert-butyl (4-((2-bromopyridin-4-yl)oxy)-2-fluorophenyl)carbamate (500 mg, 1.30 mmol), Pd₂(dba)₃ (239 mg, 0.26 mmol), BINAP (162 mg, 0.26 mmol) and t-BuONa (752 mg, 7.83 mmol) in THE (30 mL) was stirred at 75° C. for 16 hours. The mixture was diluted with water and extracted with DCM. The organic phase was concentrated in vacuum and the residue was purified by column chromatography to give the desired product (330 mg, 63.2%) as a yellow solid. LC/MS: 399.2 [M+H]⁺.

### Step 2: Preparation of 1-(4-(4-amino-3-fluorophenoxy)pyridin-2-yl)pyrrolidine-3-carbonitrile

A solution of tert-butyl (4-((2-(3-cyanopyrrolidin-1-yl)pyridin-4-yl)oxy)-2-fluoro phenyl)carbamate (235 mg, 0.59 mmol) in TFA/DCM=1:5 (6 mL) was stirred at room temperature for 2 hours. The mixture was diluted with saturated NaHCO₃ solution and extracted with DCM. The organic phase was concentrated in vacuum to give the desired product (120 mg, 68.2%) as a brown solid. LC/MS: 299 [M+H]⁺.

### Step 3: Preparation of 1-(4-(4-((6-((1-acryloylpiperidin-4-yl)amino)-7-methoxyquinazolin-4-yl)amino)-3-fluorophenoxy)pyridin-2-yl)pyrrolidine-3-carbonitrile (151)

The mixture of 1-(4-(4-amino-3-fluorophenoxy)pyridin-2-yl)pyrrolidine-3-arbonitrile (110 mg, 0.37 mmol), 1-(4-((4-chloro-7-methoxyquinazolin-6-yl)amino)piperidin-1-yl)prop-2-en-1-one (128 mg, 0.37 mmol) and TsOH (64 mg, 0.37 mmol) in MeCN was stirred at 75° C. for 16 hours. The mixture was concentrated in vacuum and the residue was purified by Pre-TLC to give the title compound (75 mg, 33%). LC/MS: 609.2 [M+H]⁺. ¹H NMR (400 MHz, DMSO-d6) b 9.33 (s, 1H), 8.26 (s, 1H), 8.06 (d, J=5.7 Hz, 1H), 7.58 (t, J=8.8 Hz, 1H), 7.31-7.02 (m, 4H), 6.91-6.81 (m, 1H), 6.33-6.06 (m, 3H), 5.69 (dd, J=10.4, 2.5 Hz, 1H), 5.40 (d, J=8.5 Hz, 1H), 4.51-4.43 (m, 1H), 4.18-4.10 (m, 1H), 3.97 (s, 3H), 3.78-3.39 (m, 6H), 3.27-3.18 (m, 1H), 2.91-2.81 (m, 1H), 2.40-2.20 (m, 2H), 2.11-2.02 (m, 2H), 1.50-1.36 (m, 2H).

### Example 34: Preparation of 1-(4-((4-((2-fluoro-4-((2-((3aR,6aS)-tetrahydro-1H-furo[3,4-c]pyrrol-5(3H)-yl)pyridin-4-yl)oxy)phenyl)amino)-7-methoxyquinazolin-6-yl)oxy)piperidin-1-yl)prop-2-en-1-one (Compound 165)

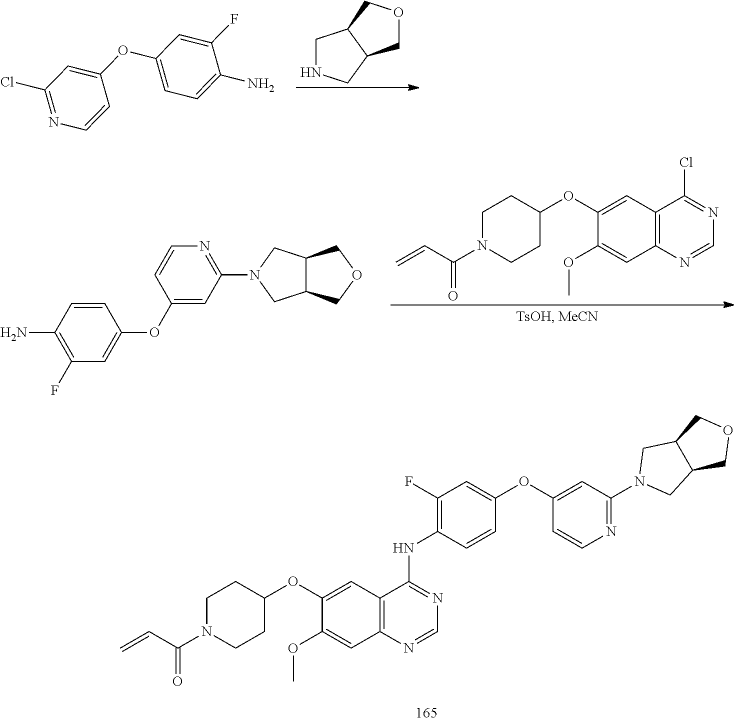

### Step 1: Preparation of 2-fluoro-4-((2-((3aR,6aS)-tetrahydro-1H-furo[3,4-c]pyrrol-5(3H)-yl)pyri din-4-yl)oxy)aniline

A solution of 4-((2-chloropyridin-4-yl)oxy)-2-fluoroaniline (200 mg, 0.83 mmol) in (3aR,6aS)-hexahydro-1H-furo[3,4-c]pyrrole (189.6 mg, 1.67 mmol) was stirred under nitrogen at 120° C. for 16 hours. The mixture was diluted with water (10 mL) and extracted with DCM (10 mL×3). The organic phase was washed with brine and dried over Na₂SO₄. The solvent was removed in vacuum and the residue was purified via silica gel column chromatography (PE:EA=1:1) to afford the desired compound (100 mg, 37.8%) as a white solid. LC/MS: 316.1[M+H]⁺.

### Step 2: Preparation of 1-(4-((4-((2-fluoro-4-((2-((3aR,6aS)-tetrahydro-1H-furo[3,4-c]pyrrol-5(3H)-yl)pyridin-4-yl)oxy)phenyl)amino)-7-methoxyquinazolin-6-yl)oxy)piperidin-1-yl)prop-2-en-1-one (165)

To a solution of 2-fluoro-4-((2-((3aR,6aS)-tetrahydro-1H-furo[3,4-c]pyrrol-5(3H)-yl)pyridin-4-yl)oxy)aniline (40 mg, 0.12 mmol) in MeCN (10 mL) was added 1-(4-((4-chloro-7-methoxyquinazolin-6-yl)oxy)piperidin-1-yl)prop-2-en-1-one (44.1 mg, 0.12 mmol) and TsOH (87.2 mg, 0.50 mmol). The reaction was stirred under nitrogen at 80° C. for 2 hours. The mixture was basified with saturated Na₂CO₃ solution. The mixture was diluted with water (10 mL) and extracted with DCM (10 mL×3). The organic phase was washed with brine and dried over Na₂SO₄. The solvent was removed in vacuum and the residue was purified by Prep-HPLC to give the titled compound (20 mg, 23.9% yield) as a white solid. LC/MS: 627.0 [M+H]⁺. ¹H NMR (400 MHz, DMSO-d6) b 9.46 (s, 1H), 8.36 (s, 1H), 8.04 (d, J=5.2 Hz, 1H), 7.90 (s, 1H), 7.62-7.54 (m, 1H), 7.27-7.15 (m, 2H), 7.04 (d, J=10.0 Hz, 1H), 6.90-6.81 (m, 1H), 6.27-6.21 (m, 1H), 6.16-6.03 (m, 2H), 5.69 (d, J=11.2 Hz, 1H), 4.81-4.74 (m, 1H), 4.02-3.87 (m, 4H), 3.86-3.78 (m, 2H), 3.60-3.42 (m, 5H), 3.30-3.24 (m, 2H), 3.04-2.94 (m, 2H), 2.10-1.98 (m, 2H), 1.75-1.67 (m, 2H), 1.30-1.20 (m, 1H), 0.92-0.77 (m, 1H).

### Example 35: Preparation of 4-(4-((6-((1-acryloylpiperidin-4-yl)amino)-7-methoxyquinazolin-4-yl)amino)-3-fluoro phenoxy)-N-(tert-butyl)picolinamide (Compound 167)

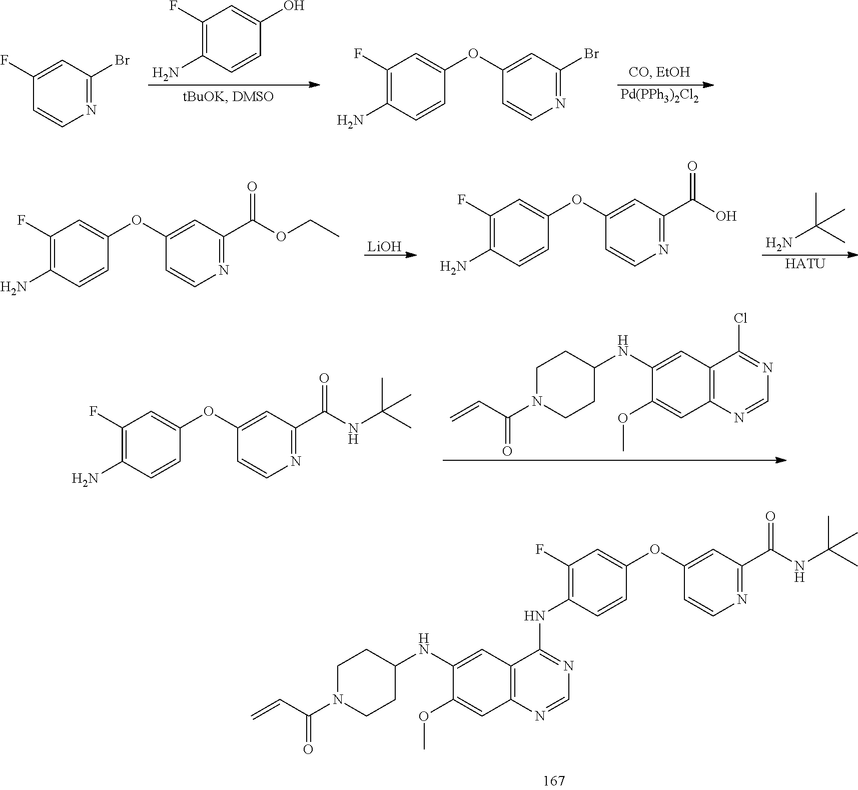

### Step 1: Preparation of 4-((2-bromopyridin-4-yl)oxy)-2-fluoroaniline

To a solution of 4-amino-3-fluorophenol (1.00 g, 7.87 mmol) in DMSO (50 mL) was added t-BuOK (971 mg, 8.65 mmol) under Ar. The reaction mixture was stirred at room temperature for 0.5 hour followed by addition of 2-bromo-4-fluoropyridine (1.45 g, 8.26 mmol). The reaction mixture was stirred at 80° C. for 2 hours. The reaction mixture was poured into water (150 mL) and extracted with EA (100 mL×3). The combined organic layers were washed with brine (200 mL×3), dried over Na₂SO₄ and concentrated under vacuum to give a crude product. The crude product was purified by Flash Chromatography (DCM=100%) to give the desired product (1.39 g, 62.4%) as an off-white solid. LC/MS: 283.0 [M+H]⁺.

### Step 2: Preparation of ethyl 4-(4-amino-3-fluorophenoxy)picolinate

To a solution of 4-((2-bromopyridin-4-yl)oxy)-2-fluoroaniline (300 mg, 1.06 mmol) in EtOH (5 mL) was added Pd(PPh3)2Cl2 (149 mg, 0.21 mmol) and Et₃N (321 mg, 3.18 mmol). The reaction mixture was stirred at 70° C. for 16 hours under CO atmosphere. After evaporation of solvent, the crude product was purified by Prep-HPLC (38% ACN in H₂O, 0.1% TFA) to give the desired product (150 mg, 51.2%) as a white solid. LC/MS: 277.1 [M+H]⁺.

### Step 3: Preparation of 4-(4-amino-3-fluorophenoxy)picolinic acid

To a solution of ethyl 4-(4-amino-3-fluorophenoxy)picolinate (190 mg, 0.69 mmol) in THF/MeOH/H₂O=3:3:1 (5 mL) was added LiOH·H₂O (87 mg, 2.06 mmol). The mixture stirred at room temperature for 1 hour. The crude product was purified by Prep-LPLC (H₂O/ACN=0˜15%, 0.1% TFA) to give the desired product (170 mg, 99.6%) as a yellow solid. LC/MS: 249.1 [M+H]⁺.

### Step 4: Preparation of 4-(4-amino-3-fluorophenoxy)-N-(tert-butyl)picolinamide

To a solution of 4-(4-amino-3-fluorophenoxy)picolinic acid (100 mg, 0.40 mmol) in DMF (6 mL) was added 2-methylpropan-2-amine (147 mg, 2.01 mmol), DIEA (156 mg, 1.21 mmol) and HATU (230 mg, 0.60 mmol). The reaction mixture was stirred at room temperature for 1 hour. The reaction mixture was poured into water (30 mL) then extracted with EA (50 mL×3). The combined organic layers were washed with brine (100 mL×2), dried over Na₂SO₄ and concentrated in vacuum to give a crude product. The crude product was purified by Flash Chromatography (DCM/MeOH=0˜6%) to give the desired product (80 mg, 65.5%) as a yellow solid. LC/MS: 304.0 [M+H]⁺.

### Step 5: Preparation of 4-(4-((6-((1-acryloylpiperidin-4-yl)amino)-7-methoxyquinazolin-4-yl) amino)-3-fluorophenoxy)-N-(tert-butyl)picolinamide (167)

To a solution of 4-(4-amino-3-fluorophenoxy)-N-(tert-butyl)picolinamide (30 mg, 0.09 mmol) in MeCN (4 mL) was added 1-(4-((4-chloro-7-methoxyquinazolin-6-yl)amino)piperidin-1-yl)prop-2-en-1-one (26 mg, 0.09 mmol) and TsOH (45 mg, 0.26 mmol). The reaction mixture was stirred at 80° C. for 2 hours. The mixture was basified with Et₃N and extracted with EA (20 mL×3). The combined organic layers were dried over Na₂SO₄ and concentrated in vacuum to give a crude product. The crude product was purified by Prep-HPLC (0.1% TFA in H₂O/CAN=20˜60%) to give the desired product (22.2 mg, 41.9%). LC/MS: 614.2 [M+H]⁺. ¹H NMR (400 MHz, DMSO-d6) b 10.76 (s, 1H), 8.74 (s, 1H), 8.59 (d, J=5.6 Hz, 1H), 8.09 (s, 1H), 7.73 (t, J=8.7 Hz, 1H), 7.52 (dd, J=10.7, 2.6 Hz, 1H), 7.44 (d, J=2.7 Hz, 2H), 7.34 (dd, J=5.6, 2.6 Hz, 1H), 7.27 (dd, J=8.6, 1.7 Hz, 1H), 7.17 (s, 1H), 6.87 (dd, J=16.7, 10.5 Hz, 1H), 6.13 (dd, J=16.7, 2.5 Hz, 1H), 6.02 (d, J=8.1 Hz, 1H), 5.70 (dd, J=10.4, 2.5 Hz, 1H), 4.49 (d, J=12.7 Hz, 1H), 4.17 (d, J=13.8 Hz, 1H), 4.05 (s, 3H), 3.56 (s, 1H), 3.22 (t, J=12.4 Hz, 1H), 2.84 (t, J=12.0 Hz, 1H), 2.05 (d, J=12.1 Hz, 2H), 1.58-1.44 (m, 2H), 1.39 (s, 9H).

### Example 36: Preparation of 4-(4-((6-((1-acryloylpiperidin-4-yl)amino)-7-methoxyquinazolin-4-yl)amino)-3-fluoro phenoxy)-N-methylpicolinamide (Compound 181)

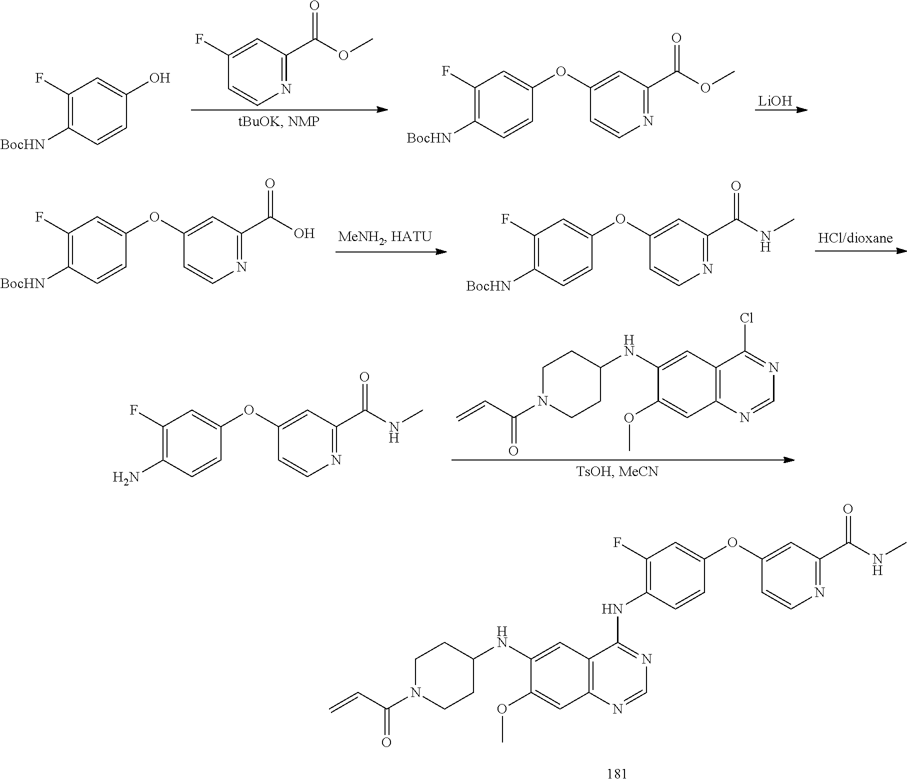

### Step 1: Preparation of methyl 4-(4-((tert-butoxycarbonyl)amino)-3-fluorophenoxy)picolinate

A solution of tert-butyl (2-fluoro-4-hydroxyphenyl)carbamate (500 mg, 2.20 mmol) in NMP (10 mL) was added t-BuOK (272 mg, 2.42 mmol) under Ar. The reaction mixture was stirred at room temperature for 0.5 hours followed by addition of methyl 4-fluoropicolinate (358 mg, 2.31 mmol). The reaction mixture was stirred at 70° C. for 2 hours. The reaction mixture was poured into water (40 mL) and extracted with EA (50 mL×3). The combined organic layers were washed with brine (100 mL×3), dried over Na₂SO₄, and concentrated in vacuum to give a crude product. The crude product was purified by Flash Chromatography (DCM/MeOH=0-8%) to give the desired product (440 mg, 55.1%) as a yellow solid. LC/MS: 362.9 [M+H]⁺.

### Step 2: Preparation of 4-(4-((tert-butoxycarbonyl)amino)-3-fluorophenoxy)picolinic acid

A solution of methyl 4-(4-((tert-butoxycarbonyl)amino)-3-fluorophenoxy)picolinate (200 mg, 0.55 mmol) in THF/MeOH/H₂O=3:3:1 (5 mL) was added LiOH·H₂O (69 mg, 1.66 mmol). The mixture was stirred at room temperature for 1 hour. The mixture was acidified with HCl (2 N) and extracted with DCM (20 mL×3). The combined organic layers were dried over Na₂SO₄ and concentrated in vacuum to give the desired product (186 mg, 96.7%) as a pink solid. LC/MS: 348.9 [M+H]⁺.

### Step 3: Preparation of tert-butyl (2-fluoro-4-((2-(methylcarbamoyl)pyridin-4-yl)oxy)phenyl) carbamate

To a solution of 4-(4-((tert-butoxycarbonyl)amino)-3-fluorophenoxy)picolinic acid (85 mg, 0.24 mmol) in DMF (6 mL) was added methylamine hydrochloride (49 mg, 0.73 mmol), DIEA (315 mg, 2.44 mmol) and HATU (186 mg, 0.49 mmol). The reaction mixture was stirred at room temperature for 16 hours. The reaction mixture was poured into water (20 mL) and extracted with EA (30 mL×3). The combined organic layers were washed with brine (50 mL×2), dried over Na₂SO₄ and concentrated in vacuum to give a crude product. The crude product was purified by Flash Chromatography (DCM/MeOH=0-20%) to give the desired product (20 mg, 22.8%) as a yellow solid. LC/MS: 362.0 [M+H]⁺.

### Step 4: Preparation of 4-(4-amino-3-fluorophenoxy)-N-methylpicolinamide

A solution of tert-butyl (2-fluoro-4-((2-(methylcarbamoyl)pyridin-4-yl)oxy)phenyl)carbamate (15 mg, 0.04 mmol) in HCl/dioxane (4 N, 5 mL) was stirred at room temperature for 2 hours. The reaction mixture was concentrated in vacuum to give the desired product (10 mg, 92.3%) as a yellow solid. LC/MS: 262.0 [M+H]⁺.

### Step 5: Preparation of 4-(4-((6-((1-acryloylpiperidin-4-yl)amino)-7-methoxyquinazolin-4-yl)amino)-3-fluorophenoxy)-N-methylpicolinamide (181)

To a solution of 4-(4-amino-3-fluorophenoxy)-N-methylpicolinamide (10 mg, 0.04 mmol) in MeCN (4 mL) was added 1-(4-((4-chloro-7-methoxyquinazolin-6-yl)amino)piperidin-1-yl)prop-2-en-1-one (13 mg, 0.04 mmol) and TsOH (20 mg, 0.11 mmol). The reaction mixture was stirred at 80° C. for 2 hours. The mixture was basified with Et₃N and extracted with EA (20 mL×3). The combined organic layers were dried over Na₂SO₄ and concentrated in vacuum to give a crude product. The crude product was purified by Prep-HPLC (0.1% TFA in H2O/ACN=20%-60%) to give the desired product (10.5 mg, 48.0%) as a white solid. LC/MS: 572.0 [M+H]⁺. ¹H NMR (400 MHz, DMSO-d6) b 10.85 (s, 1H), 8.85 (d, J=4.8 Hz, 1H), 8.76 (s, 1H), 8.61 (d, J=5.6 Hz, 1H), 7.73 (t, J=8.7 Hz, 1H), 7.53 (dd, J=10.7, 2.6 Hz, 1H), 7.46 (d, J=2.6 Hz, 2H), 7.31 (dd, J=5.6, 2.6 Hz, 1H), 7.27 (dd, J=8.7, 1.8 Hz, 1H), 7.19 (s, 1H), 6.88 (dd, J=16.7, 10.5 Hz, 1H), 6.13 (dd, J=16.7, 2.4 Hz, 1H), 6.06 (d, J=7.9 Hz, 1H), 5.70 (dd, J=10.4, 2.4 Hz, 1H), 4.49 (d, J=13.2 Hz, 1H), 4.17 (d, J=12.3 Hz, 1H), 4.05 (s, 3H), 3.78 (s, 1H), 3.22 (t, J=12.4 Hz, 1H), 2.93-2.78 (m, 4H), 2.08-2.01 (m, 2H), 1.57-1.42 (m, 2H).

### Example 37: Preparation of 1-(4-((4-((2-fluoro-4-((2-((tetrahydro-2H-pyran-4-yl)amino)pyridin-4-yl)oxy)phenyl) amino)-7-methoxyquinazolin-6-yl)amino)piperidin-1-yl)prop-2-en-1-one (Compound 185)

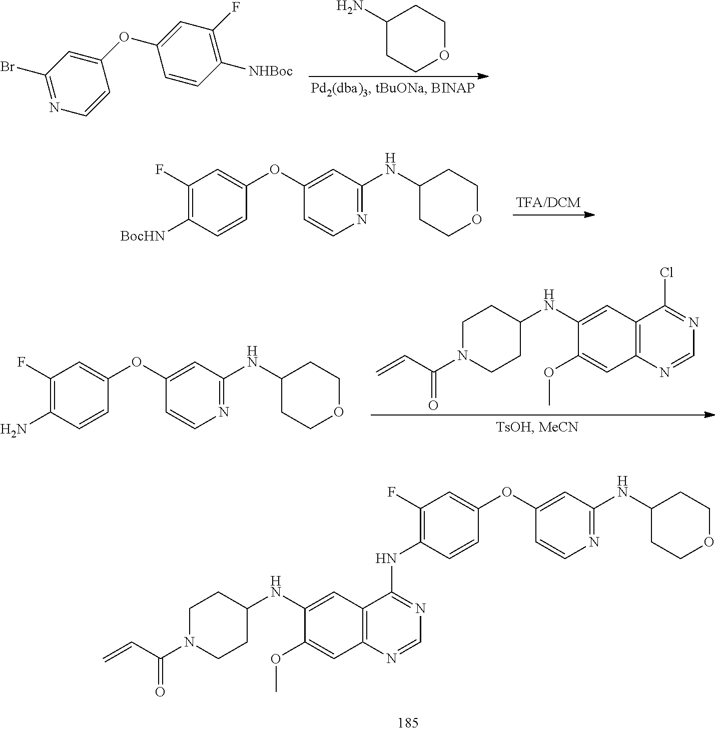

### Step 1: Preparation of tert-butyl (2-fluoro-4-((2-((tetrahydro-2H-pyran-4-yl)amino)pyridin-4-yl)oxy)phenyl)carbamate

To a solution of tert-butyl (4-((2-bromopyridin-4-yl)oxy)-2-fluorophenyl)carbamate (200 mg, 0.52 mmol) in THE (10 mL) was added tetrahydro-2H-pyran-4-amine (158.3 mg, 1.56 mmol), t-BuONa (150.5 mg, 1.56 mmol), BINAP (64.9 mg, 0.10 mmol) and Pd₂(dba)₃ (95.6 mg, 0.10 mmol). The reaction was stirred at 70° C. for 16 hours under nitrogen. The mixture was diluted with water (10 mL) and extracted with DCM (10 mL×3). The organic phase was washed with brine and dried over Na₂SO₄. The solvent was removed in vacuum and the residue was purified via silica gel column chromatography (PE:EA=1:1) to afford the desired compound (100 mg, 47.4%) as a white solid. LC/MS: 404.2 [M+H]⁺.

### Step 2: Preparation of 4-(4-amino-3-fluorophenoxy)-N-(tetrahydro-2H-pyran-4-yl)pyridin-2-amine

A solution of tert-butyl (4-((2-bromopyridin-4-yl)oxy)-2-fluorophenyl)carbamate (90 mg, 0.22 mmol) in DCM/TFA (1:3, 5 mL) was stirred at room temperature for 1 hour. The mixture was basified with Na₂CO₃ solution. The mixture was diluted with water (10 mL) and extracted with DCM (10 mL×3). The organic phase was washed with brine and dried over Na₂SO₄. The solvent was removed in vacuum to afford the desired compound (60 mg, 89.9%) as a white solid. LC/MS: 304.2[M+H]⁺.

### Step 3: Preparation of 1-(4-((4-((2-fluoro-4-((2-((tetrahydro-2H-pyran-4-yl)amino)pyridin-4-yl)oxy)phenyl)amino)-7-methoxyquinazolin-6-yl)amino)piperidin-1-yl)prop-2-en-1-one (185)

A solution of 4-(4-amino-3-fluorophenoxy)-N-(tetrahydro-2H-pyran-4-yl)pyridin-2-amine (50 mg, 0.16 mmol) in MeCN (3 mL) was added 1-(4-((4-chloro-7-methoxyquinazolin-6-yl)amino) piperidin-1-yl)prop-2-en-1-one (57.2 mg, 0.16 mmol) and TsOH (85.14 mg, 0.49 mmol). The reaction was stirred under nitrogen at 80° C. for 3 hours. The mixture basified with Na₂CO₃ solution. The mixture was diluted with water (10 mL) and extracted with DCM (10 mL×3). The organic phase was washed with brine and dried over Na₂SO₄. The solvent was removed in vacuum and the residue was purified by Prep-HPLC to give the title compound (33 mg, 31%) as a white solid. LC/MS: 614.1 [M+H]⁺. ¹H NMR (400 MHz, DMSO) b 9.18 (s, 1H), 8.23 (s, 1H), 7.91 (d, J=5.6 Hz, 1H), 7.58 (t, J=8.8 Hz, 1H), 7.26 (s, 1H), 7.23-7.19 (m, 1H), 7.08 (s, 1H), 7.05-7.01 (m, 1H), 6.86 (dd, J=16.8, 10.4 Hz, 1H), 6.63 (d, J=7.6 Hz, 1H), 6.19 (dd, J=5.6, 2.4 Hz, 1H), 6.12 (dd, J=16.8, 2.4 Hz, 1H), 6.00 (d, J=2.0 Hz, 1H), 5.69 (dd, J=10.4, 2.4 Hz, 1H), 5.33 (d, J=8.4 Hz, 1H), 4.45 (d, J=12.0 Hz, 1H), 4.13 (d, J=11.6 Hz, 1H), 3.96 (s, 3H), 3.86-3.81 (m, 2H), 3.41-3.35 (m, 3H), 3.28-3.18 (m, 2H), 2.86 (t, J=12.0 Hz, 1H), 2.10-2.03 (m, 2H), 1.89-1.82 (m, 2H), 1.49-1.31 (m, 4H).

### Example 38: 1-(4-((4-((2-fluoro-4-((2-(3-methyl-1,2,4-oxadiazol-5-yl)pyridin-4-yl)oxy)phenyl)amino)-7-methoxyquinazolin-6-yl)amino)piperidin-1-yl)prop-2-en-1-one (Compound 191)

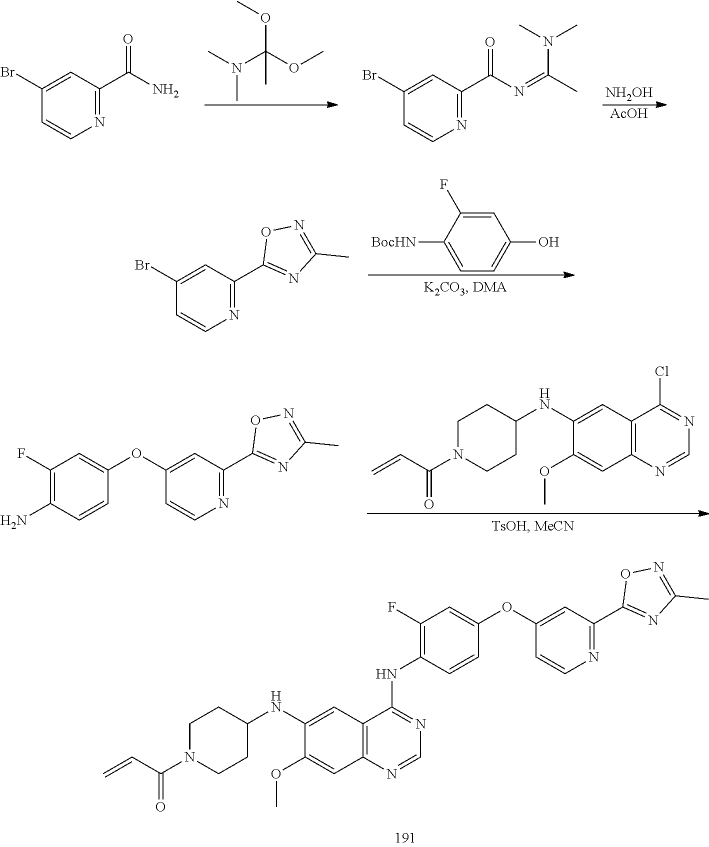

### Step 1: Preparation of 4-bromo-N-(1-(dimethylamino)ethylidene)picolinamide

A solution of 4-bromopicolinamide (200 mg, 0.99 mmol) in 1,1-dimethoxy-N, N-dimethylethan-1-amine (0.5 mL) was stirred at room temperature for 2 hours. The solvent was removed in vacuum to give the product (200 mg, crude) as a yellow solid. LC/MS: 270.0 [M+H]⁺

### Step 2: Preparation of 5-(4-bromopyridin-2-yl)-3-methyl-1,2,4-oxadiazole

To a solution of 4-bromo-N-(1-(dimethylamino)ethylidene)picolinamide (150 mg, crude) in dioxane (2 mL) was added hydroxylamine (29.2 mg, 0.83 mmol) and AcOH (2 mL). The reaction was stirred at 90° C. for 1 hour. The solvent was removed in vacuum. The residue was dissolved in EA (10 mL) and washed with NaHCO₃. The organic phase was concentrated in vacuum. The residue was purified by Pre-TLC with PE:EA=2:1 to give the desired product (100 mg, 56.1% yield for two steps) as a white solid. LC/MS: 239.9 [M+H]⁺.

### Step 3: Preparation of 2-fluoro-4-((2-(3-methyl-1,2,4-oxadiazol-5-yl) pyridin-4-yl)oxy) aniline

To a solution of 5-(4-bromopyridin-2-yl)-3-methyl-1,2,4-oxadiazole (45 mg, 0.18 mmol) in DMA (10 mL) was added tert-butyl (2-fluoro-4-hydroxyphenyl) carbamate (55 mg, 0.24 mmol) and K₂CO₃ (42 mg, 0.37 mmol). The reaction mixture was stirred at 140° C. for 1 hour. LC-MS analysis indicated the Boc group was cleaved during reaction. The mixture was diluted with water and extracted with EA. The organic phase was washed with brine and dried over Na₂SO₄. The solvent was removed in vacuum and the residue was purified by Prep-HPLC to afford the desired compound (25 mg, 35.9%) as a yellow solid. LC/MS: 287.1 [M+H]⁺.

### Step 4: Preparation of 1-(4-((4-((2-fluoro-4-((2-(3-methyl-1,2,4-oxadiazol-5-yl) pyridin-4-yl) oxy) phenyl) amino)-7-methoxyquinazolin-6-yl) amino) piperidin-1-yl) prop-2-en-1-one (191)

To a solution of 2-fluoro-4-((2-(3-methyl-1,2,4-oxadiazol-5-yl) pyridin-4-yl) oxy) aniline (25 mg, 0.08 mmol) in MeCN (5 mL) was added 1-(4-((4-chloro-7-methoxyquinazolin-6-yl) amino) piperidin-1-yl) prop-2-en-1-one (30 mg, 0.08 mmol) and TsOH (30 mg, 0.17 mmol). The reaction mixture was stirred at 80° C. for 2 hours. The reaction was washed with saturated sodium bicarbonate aqueous solution and dried over Na₂SO₄. The organic phase was evaporated in vacuum and the residue was purified by Prep-HPLC to afford the desired compound (13.6 mg, 26.1%) as a yellow solid. LC/MS: 597.2[M+H]⁺. ¹H NMR (400 MHz, DMSO-d6) b 9.47 (s, 1H), 8.76 (d, J=5.7 Hz, 1H), 8.32 (s, 1H), 7.70 (t, J=8.7 Hz, 1H), 7.64 (d, J=2.4 Hz, 1H), 7.46 (dd, J=10.7, 2.6 Hz, 1H), 7.39 (dd, J=5.6, 2.5 Hz, 1H), 7.30 (s, 1H), 7.23 (dd, J=8.7, 1.7 Hz, 1H), 7.10 (s, 1H), 6.87 (dd, J=16.7, 10.5 Hz, 1H), 6.12 (dd, J=16.7, 2.5 Hz, 1H), 5.69 (dd, J=10.4, 2.5 Hz, 1H), 5.46 (d, J=8.4 Hz, 1H), 4.47 (d, J=13.4 Hz, 1H), 4.15 (d, J=13.0 Hz, 1H), 3.98 (s, 3H), 3.77 (d, J=8.7 Hz, 1H), 3.27-3.20 (m, 1H), 2.86 (t, J=12.2 Hz, 1H), 2.43 (s, 3H), 2.15-2.03 (m, 2H), 1.54-1.42 (m, 2H).

### Example 39: Preparation of 1-(4-((4-((2-fluoro-4-((2-(5-methyl-1,3,4-oxadiazol-2-yl)pyridin-4-yl)oxy)phenyl)amino)-7-methoxyquinazolin-6-yl)amino)piperidin-1-yl)prop-2-en-1-one (Compound 193)

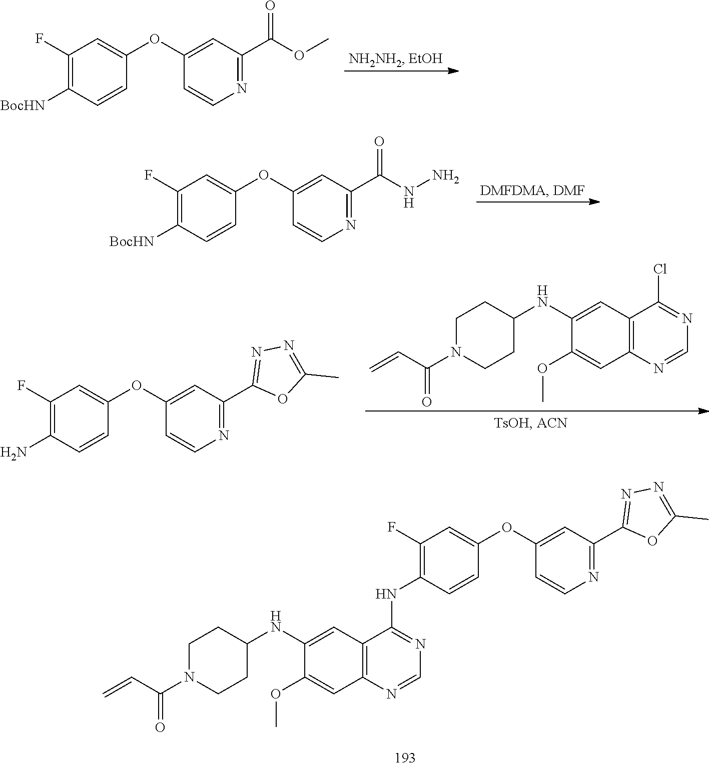

### Step 1: Preparation of tert-butyl (2-fluoro-4-((2-(hydrazinecarbonyl)pyridin-4-yl)oxy)phenyl) carbamate

To a solution of methyl 4-(4-((tert-butoxycarbonyl)amino)-3-fluorophenoxy)picolinate (250 mg, 0.69 mmol) in EtOH (2 mL) stirred at room temperature in a sealed tube was added N₂H₄—H₂O (2 mL). The reaction was stirred at 90° C. for 70 minutes. The mixture was cooled to room temperature and concentrated in vacuum to give the crude product (250 mg) which was used in the next step without further purification. LC/MS: 363.1 [M+H]⁺.

### Step 2: Preparation of 2-fluoro-4-((2-(5-methyl-1,3,4-oxadiazol-2-yl)pyridin-4-yl)oxy)aniline

To a solution of tert-butyl (2-fluoro-4-((2-(hydrazinecarbonyl)pyridin-4-yl)oxy)phenyl)carbamate (250 mg) in DMF (5 mL) stirred at room temperature was added 1,1-dimethoxy-N,N-dimethylethan-1-amine (138 mg, 1.035 mmol). The reaction mixture was stirred at 60° C. for 2.5 hours. The mixture was cooled to room temperature and concentrated in vacuum. The residue was dissolved with toluene and TsOH (119 mg, 0.69 mmol) was added.

The reaction was stirred at 110° C. for another 3 hours. The mixture was cooled to room temperature, concentrated in vacuum to give a crude product. The crude product was purified by flash chromatography (10% MeOH in DCM) to give the desired product (70 mg, 35.4%). LC/MS: 287.1 [M+H]⁺.

### Step 3: Preparation of 1-(4-((4-((2-fluoro-4-((2-(5-methyl-1,3,4-oxadiazol-2-yl)pyridin-4-yl)oxy) phenyl)amino)-7-methoxyquinazolin-6-yl)amino)piperidin-1-yl)prop-2-en-1-one (193)

To a solution of 2-fluoro-4-((2-(5-methyl-1,3,4-oxadiazol-2-yl)pyridin-4-yl)oxy)aniline (60 mg, 0.21 mmol) in CH₃CN (10 mL) stirred at room temperature was added 1-(4-((4-chloro-7-methoxyquinazolin-6-yl)amino)piperidin-1-yl)prop-2-en-1-one (73 mg, 0.21 mmol) and TsOH (108 mg, 0.63 mmol). The reaction mixture was stirred at 80° C. for 2 hours. The mixture was basified with Et₃N and concentrated in vacuum to give a crude product. The crude product was purified by Prep-TLC (DCM:MeOH=10:1) to give the desired product (30 mg, 24.0%). LC/MS: 597.0 [M+H]⁺. ¹H NMR (400 MHz, DMSO-d6) δ=9.45 (s, 1H), 8.70 (d, J=5.6 Hz, 1H), 8.31 (s, 1H), 7.70 (t, J=8.8 Hz, 1H), 7.59 (d, J=2.0 Hz, 1H), 7.44 (dd, J=10.8, 2.4, 1H), 7.34-7.27 (m, 2H), 7.25-7.16 (m, 1H), 7.10 (s, 1H), 6.87 (dd, J=16.8, 10.4 Hz, 1H), 6.12 (dd, J=16.8, 2.4 Hz, 1H), 5.69 (dd, J=10.4, 2.4 Hz, 1H), 5.42 (d, J=8.8 Hz, 1H), 4.47 (d, J=12.4 Hz, 1H), 4.14 (d, J=13.2 Hz, 1H), 3.98 (s, 3H), 3.84-3.72 (m, 1H), 3.27-3.18 (m, 1H), 2.89-2.84 (m, 1H), 2.61 (s, 3H), 2.15-2.03 (m, 2H), 1.53-1.41 (m, 2H).

### Example 40: Preparation of 1-(4-((4-((2-fluoro-4-((2-(morpholinomethyl)pyridin-4-yl)oxy)phenyl)amino)-7-methoxy quinazolin-6-yl)amino)piperidin-1-yl)prop-2-en-1-one (Compound 210)

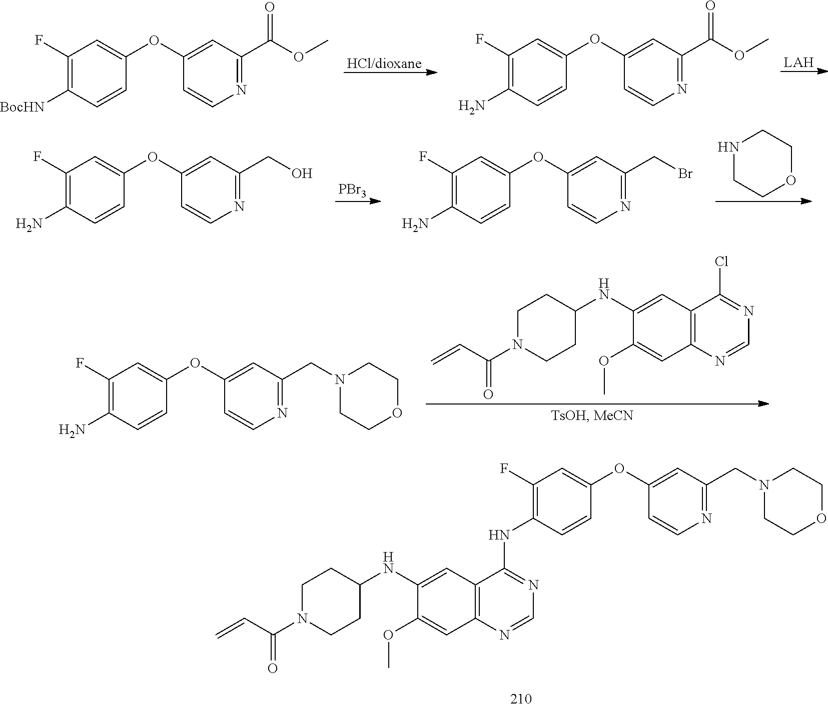

### Step 1: Preparation of methyl 4-(4-amino-3-fluorophenoxy)picolinate

A solution of methyl 4-(4-((tert-butoxycarbonyl)amino)-3-fluorophenoxy)picolinate (700 mg, 1.93 mmol) in HCl/dioxane (4 N, 5 mL) was stirred at room temperature for 1 hour. The solvent was removed in vacuum. The residue was dissolved with saturated Na₂CO₃ solution and extracted with DCM. The organic phase was concentrated to give the desired compound (300 mg, 59.2%) as a yellow solid. LC/MS: 263.0 [M+H]⁺.

### Step 2: Preparation of (4-(4-amino-3-fluorophenoxy)pyridin-2-yl)methanol

To a solution of methyl 4-(4-amino-3-fluorophenoxy)picolinate (300 mg, 1.14 mmol) in THE (10 mL) was added LAH (119 mg, 3.15 mmol) at 0° C. under N₂ atmosphere. The reaction was warmed to room temperature and stirred for 1 hour. The reaction was quenched with water (0.12 mL), NaOH (0.12 mL, 15% in water) and water (0.36 mL). The solid was filtered off and the solution was concentrated in vacuum to give a crude product. The crude product was purified by flash with PE:EA=2:1 to give the desired compound (150 mg, 56.1%) as a yellow solid. LC/MS: 235.1 [M+H]⁺.

### Step 3: Preparation of 4-((2-(bromomethyl)pyridin-4-yl)oxy)-2-fluoroaniline

To a solution of (4-(4-amino-3-fluorophenoxy)pyridin-2-yl)methanol (150 mg, 0.64 mmol) in DCE (10 mL) was added PBr₃ (1.06 g, 3.92 mmol). The reaction was stirred at 50° C. for 2 hours. The reaction was quenched with NaHCO₃ solution and extracted with DCM (10 mL×3). The organic phase was washed with brine, dried over Na₂SO₄ and concentrated in vacuum. The residue was purified by flash chromatography with DCM:MeOH=10:1 to give the desired product (80 mg, 42.1%). LC/MS: 297.0 [M+H]⁺.

### Step 4: Preparation of 2-fluoro-4-((2-(morpholinomethyl)pyridin-4-yl)oxy)aniline

A solution of 4-((2-(bromomethyl)pyridin-4-yl)oxy)-2-fluoroaniline (80 mg, 0.27 mmol) in morpholine (2 mL) was stirred at room temperature for 17 hours. The solvent was removed in vacuum and the residue was purified by Prep-TLC with DCM:MeOH=10:1 to give the desired product (50 mg, 61.1%) as a yellow solid. LC/MS: 304.2 [M+H]⁺.

### Step 5: Preparation of 1-(4-((4-((2-fluoro-4-((2-(morpholinomethyl)pyridin-4-yl)oxy)phenyl) amino)-7-methoxyquinazolin-6-yl)amino)piperidin-1-yl)prop-2-en-1-one (210)

To a solution of 2-fluoro-4-((2-(morpholinomethyl)pyridin-4-yl)oxy)aniline (25 mg, 0.08 mmol) in MeCN (2 mL) was added 1-(4-((4-chloro-7-methoxyquinazolin-6-yl)amino)piperidin-1-yl) prop-2-en-1-one (28.6 mg, 0.08 mmol) and TsOH (42.5 mg, 0.25 mmol). The reaction was stirred at 80° C. for 2 hours. The reaction was basified with NaHCO₃ solution and extracted with DCM:MeOH=10:1 (10 mL×3). The organic phase was washed with brine, dried over Na₂SO₄ and concentrated in vacuum. The residue was purified by Prep-TLC with DCM:MeOH=10:1 to give the desired product (10 mg, 19.7%). LC/MS: 614.3 [M+H]⁺. ¹H NMR (400 MHz, DMSO) b 9.22 (s, 1H), 8.44 (d, J=5.7 Hz, 1H), 8.23 (s, 1H), 7.62 (t, J=8.8 Hz, 1H), 7.31 (dd, J=10.9, 2.6 Hz, 1H), 7.27 (s, 1H), 7.11-7.06 (m, 3H), 6.91 (dd, J=5.7, 2.5 Hz, 1H), 6.88-6.82 (m, 1H), 6.12 (dd, J=16.7, 2.5 Hz, 1H), 5.69 (dd, J=10.4, 2.5 Hz, 1H), 5.35 (d, J=8.6 Hz, 1H), 4.46 (d, J=12.9 Hz, 1H), 4.14 (d, J=12.5 Hz, 1H), 3.97 (s, 3H), 3.79-3.73 (m, 1H), 3.63-3.55 (m, 6H), 3.23 (t, J=12.2 Hz, 1H), 2.86 (t, J=11.8 Hz, 1H), 2.44-2.39 (m, 4H), 2.12-2.01 (m, 2H), 1.48-1.41 (m, 2H).

### Example 41: Preparation of 1-(4-((4-((2-fluoro-4-((2-(4-methyl-1H-pyrazol-1-yl)pyridin-4-yl)oxy)phenyl)amino)-7-methoxyquinazolin-6-yl)amino)piperidin-1-yl)prop-2-en-1-one (Compound 232)

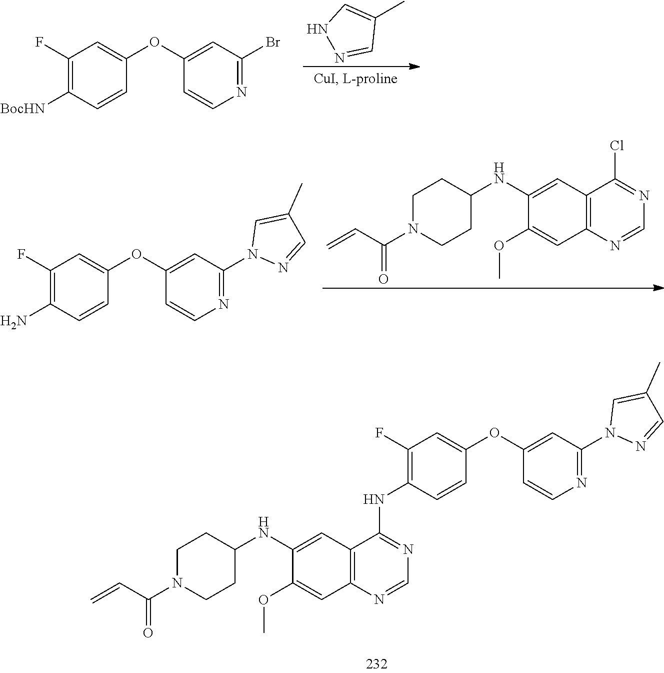

### Step 1: Preparation of tert-butyl (2-fluoro-4-((2-(4-methyl-1H-pyrazol-1-yl)pyridin-4-yl)oxy) phenyl)carbamate

To a solution of tert-butyl (4-((2-bromopyridin-4-yl)oxy)-2-fluorophenyl)carbamate (200 mg, 0.52 mmol) in DMSO (10 mL) was added 4-methyl-1H-pyrazole (214 mg, 2.61 mmol), K₂CO₃ (216 mg, 1.57 mmol), L-Proline (12 mg, 0.10 mmol) and CuI (10 mg, 0.05 mmol) under Ar. The reaction mixture was stirred at 90° C. for 3 days. LC-MS analysis indicated the Boc group was cleaved during the reaction. The reaction mixture was poured into water (40 mL) and extracted with EA (50 mL×3). The combined organic layers were washed with brine (100 mL×3), dried over Na₂SO₄ and concentrated in vacuum to give a crude product. The crude product was purified by Pre-TLC (DCM=100%) to give the desired product (40 mg, 20.3%) as a yellow solid. LC/MS: 285.1 [M+H]⁺.

### Step 2: Preparation of 1-(4-((4-((2-fluoro-4-((2-(4-methyl-1H-pyrazol-1-yl)pyridin-4-yl)oxy) phenyl)amino)-7-methoxyquinazolin-6-yl)amino)piperidin-1-yl)prop-2-en-1-one (232)

To a solution of 2-fluoro-4-((2-(4-methyl-1H-pyrazol-1-yl)pyridin-4-yl)oxy)aniline (20 mg, 0.069 mmol) in MeCN (5 mL) was added 1-(4-((4-chloro-7-methoxyquinazolin-6-yl)amino)piperidin-1-yl)prop-2-en-1-one (24 mg, 0.069 mmol) and TsOH (60 mg, 0.34 mmol) under argon. The mixture was stirred at 80° C. for 1 hour. The reaction mixture was cooled to room temperature, basified with triethylamine, diluted with H₂O (50 mL) and extracted with EtOAc (50 mL×3). The organic phase was concentrated in vacuum to give a crude product. The crude product was purified by Pre-TLC with DCM/MeOH=10:1 to give the desired product (16 mg, 36.9% yield). LC/MS: 594.8[M+H]⁺. ¹H NMR (400 MHz, DMSO-d6) b 9.23 (s, 1H), 8.39 (d, J=5.7 Hz, 2H), 8.25 (s, 1H), 7.66 (d, J=8.8 Hz, 1H), 7.62 (s, 1H), 7.42 (dd, J=10.7, 2.6 Hz, 1H), 7.27 (d, J=2.5 Hz, 2H), 7.20 (dd, J=8.6, 1.8 Hz, 1H), 7.09 (s, 1H), 7.02 (dd, J=5.7, 2.4 Hz, 1H), 6.91-6.83 (m, 1H), 6.12 (dd, J=16.7, 2.5 Hz, 1H), 5.69 (dd, J=10.4, 2.5 Hz, 1H), 5.36 (d, J=8.5 Hz, 1H), 4.47 (d, J=13.5 Hz, 1H), 4.14 (d, J=12.6 Hz, 1H), 3.97 (s, 3H), 3.80-3.73 (m, 1H), 3.27-3.20 (m, 1H), 2.91-2.83 (m, 1H), 2.14-2.03 (m, 5H), 1.51-1.40 (m, 2H).

### Example 42: Preparation of 1-(4-((4-((2-fluoro-4-((2-phenylpyridin-4-yl)oxy)phenyl)amino)-7-methoxyquinazolin-6-yl)amino)piperidin-1-yl)prop-2-en-1-one (Compound 233)

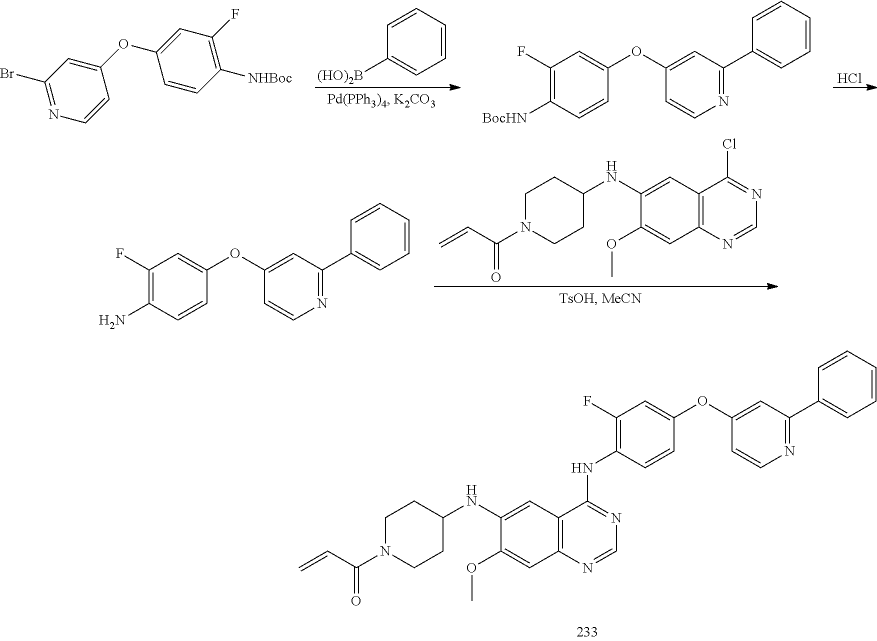

### Step 1: Preparation of tert-butyl (2-fluoro-4-((2-phenylpyridin-4-yl)oxy)phenyl)carbamate

To a solution of tert-butyl N-{4-[(2-bromopyridin-4-yl)oxy]-2-fluorophenyl}carbamate (200 mg, 0.522 mmol) in EtOH/H₂O (10.0/2.0 mL) stirred at room temperature was added phenylboronic acid (95.4 mg, 0.783 mmol), Pd(PPh₃)₄ (60.3 mg, 0.052 mmol) and K₂CO₃ (216.4 mg, 1.566 mmol). The reaction mixture was stirred under nitrogen at 80° C. overnight. The solvent was removed in vacuum and the residue was extracted with DCM (150 mL×3). The organic phase was washed with brine and dried over Na₂SO₄. The organic solvent was removed in vacuum to give a crude product. The crude product was purified by silica-gel column chromatography and eluted with 0-30% EA in PE to give the title compound (175 mg, 70.5% yield) as a yellow solid. LC/MS: 381.2 [M+H]⁺.

### Step 2: Preparation of 2-fluoro-4-((2-phenylpyridin-4-yl)oxy)aniline

A solution of tert-butyl (2-fluoro-4-((2-phenylpyridin-4-yl)oxy)phenyl)carbamate (175 mg, 80% purity, 0.388 mmol) in HCl/dioxane (4 N, 6 mL) was stirred at room temperature for 2 hours. The solvent was removed in vacuum and the residue was dissolved with DCM (150 mL). The organic phase was washed with brine and dried over Na₂SO₄. The crude product was purified by silica-gel column chromatography and eluted with 0-10% MeOH in DCM to give the title compound (110 mg, 91.0% yield) as a white solid. LC/MS: 280.9 [M+H]⁺.

### Step 3: Preparation of 1-(4-((4-((2-fluoro-4-((2-phenylpyridin-4-yl)oxy)phenyl)amino)-7-meth oxyquinazolin-6-yl)amino)piperidin-1-yl)prop-2-en-1-one (233)

To a solution of 2-fluoro-4-((2-phenylpyridin-4-yl)oxy)aniline (40 mg, 0.143 mmol) in MeCN (5 mL) stirred under argon at room temperature was added 1-{4-[(4-chloro-7-methoxyquinazolin-6-yl)amino]piperidin-1-yl}prop-2-en-1-one (49.5 mg, 0.143 mmol) and TsOH (73.6 mg, 0.428 mmol). The reaction mixture was stirred at 80° C. for 2 hours. The reaction mixture was cooled to room temperature, basified with triethylamine, diluted with H₂O (50 mL), and extracted with EtOAc (50 mL×3). The organic phase was concentrated in vacuum to give a crude product. The crude product was purified by silica-gel column chromatography with MeOH/DCM=0-10% and repurified by Prep-HPLC to give the title compound (12 mg, 14.0%) as a yellow solid. LC/MS: 591.1 [M+H]⁺. 1H NMR (400 MHz, DMSO-d6) b 10.81 (brs, 1H), 8.73 (s, 1H), 8.65 (d, J=5.6 Hz, 1H), 8.10-7.97 (m, 2H), 7.73-7.62 (m, 2H), 7.54-7.44 (m, 4H), 7.25 (d, J=8.6 Hz, 1H), 7.15 (s, 1H), 7.01-6.98 (m, 1H), 6.88 (dd, J=16.7, 10.5 Hz, 1H), 6.13 (dd, J=16.7, 2.5 Hz, 1H), 6.05 (d, J=7.2 Hz, 1H), 5.70 (dd, J=10.4, 2.5 Hz, 1H), 4.53-4.44 (m, 2H), 4.20-4.12 (m, 1H), 4.05 (s, 3H), 3.28-3.17 (m, 2H), 2.88-2.79 (m, 1H), 2.08-1.99 (m, 2H), 1.57-1.44 (m, 2H).

### Example 43: Preparation of 1-(4-((4-((4-((2-(cyclobutylamino)pyridin-4-yl)oxy)-2-fluorophenyl)amino)-7-methoxyquinazolin-6-yl)amino)piperidin-1-yl)prop-2-en-1-one (Compound 243)

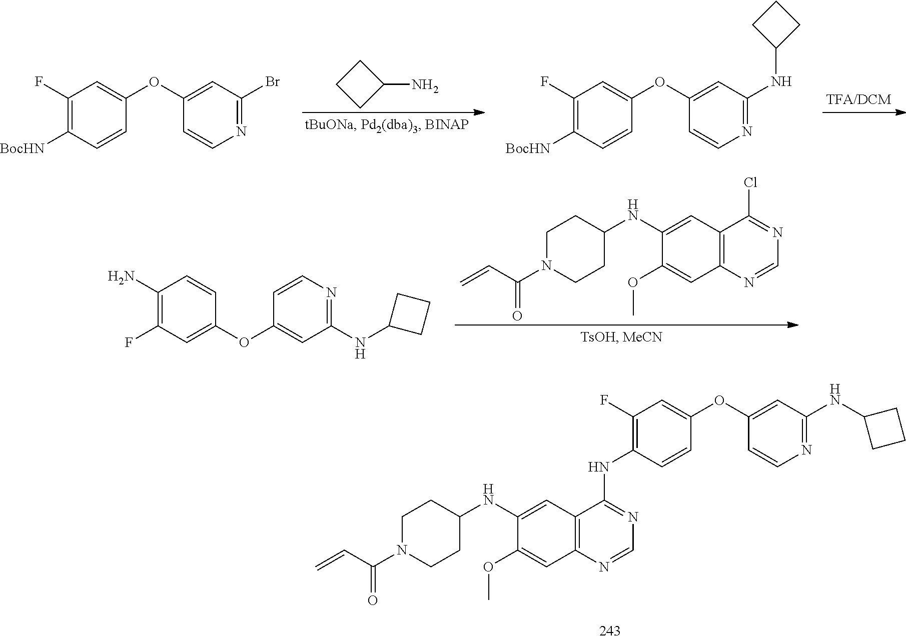

### Step 1: Preparation of tert-butyl (4-((2-(cyclobutylamino)pyridin-4-yl)oxy)-2-fluorophenyl)carbamate

The mixture of cyclobutylamine (186 mg, 2.62 mmol), tert-butyl (4-((2-bromopyridin-4-yl)oxy)-2-fluorophenyl)carbamate (200 mg, 0.52 mmol), Pd₂(dba)₃ (48 mg, 0.052 mmol), BINAP (65 mg, 0.10 mmol) and t-BuONa (151 mg, 1.57 mmol) in THE (10 mL) was stirred at 90° C. for 16 hours under N₂. The reaction mixture was diluted with water and extracted with DCM. The organic phase was concentrated in vacuum and the residue was purified by column chromatography with PE:EA=1: 0-1:1 to give the title compound (50 mg, 25.6%) as a brown solid. LC/MS: 374.1 [M+H]⁺.

### Step 2: Preparation of 4-(4-amino-3-fluorophenoxy)-N-cyclobutylpyridin-2-amine

The solution of tert-butyl (4-((2-(cyclobutylamino)pyridin-4-yl)oxy)-2-fluorophenyl) carbamate (50 mg, 0.13 mmol) in DCM:TFA=5:1 (3 mL) was stirred at room temperature for 2 hours. The mixture was basified with sat. NaHCO₃ solution and extracted with DCM. The organic phase was concentrated in vacuum to give the title compound (35 mg, 95.6%) as a brown solid. LC/MS: 274.2 [M+H]⁺.

### Step 3: Preparation of 1-(4-((4-((4-((2-(cyclobutylamino)pyridin-4-yl)oxy)-2-fluoro phenyl)amino)-7-methoxyquinazolin-6-yl)amino)piperidin-1-yl)prop-2-en-1-one (243)

A solution of 4-(4-amino-3-fluorophenoxy)-N-cyclobutylpyridin-2-amine (35 mg, 0.13 mmol), 1-(4-((4-chloro-7-methoxyquinazolin-6-yl)amino)piperidin-1-yl)prop-2-en-1-one (45 mg, 0.13 mmol) and TsOH (22 mg, 0.13 mmol) in MeCN (5 mL) was stirred at 80° C. for 16 hours. The mixture was concentrated in vacuum and the residue was purified by Pre-TLC to give the titled compound (39 mg, 45.6%). LC/MS: 584.1 [M+H]⁺. 1H NMR (400 MHz, DMSO-d6) b 9.27 (s, 1H), 8.25 (s, 1H), 7.91 (d, J=5.8 Hz, 1H), 7.58 (t, J=8.8 Hz, 1H), 7.28 (s, 1H), 7.22 (dd, J=11.0, 2.6 Hz, 1H), 7.08 (s, 1H), 7.04 (dd, J=8.6, 1.8 Hz, 1H), 6.95 (d, J=7.2 Hz, 1H), 6.87 (dd, J=16.7, 10.5 Hz, 1H), 6.19 (dd, J=5.8, 2.2 Hz, 1H), 6.12 (dd, J=16.7, 2.5 Hz, 1H), 5.93 (d, J=2.2 Hz, 1H), 5.69 (dd, J=10.4, 2.5 Hz, 1H), 5.37 (d, J=8.5 Hz, 1H), 4.51-4.41 (m, 1H), 4.29-4.19 (m, 1H), 4.18-4.08 (m, 1H), 3.97 (s, 3H), 3.82-3.71 (m, 1H), 3.23 (t, J=12.0 Hz, 1H), 2.86 (t, J=12.0 Hz, 1H), 2.29-2.18 (m, 2H), 2.13-2.01 (m, 2H), 1.90-1.77 (m, 2H), 1.71-1.58 (m, 2H), 1.51-1.37 (m, 2H).

### Example 44: Preparation of 1-(4-((4-((4-((2-((3R,4S)-3,4-dimethoxypyrrolidin-1-yl)pyrimidin-4-yl)oxy)-2-fluorophenyl)amino)-7-methoxyquinazolin-6-yl)amino)piperidin-1-yl)prop-2-en-1-one (Compound 260)

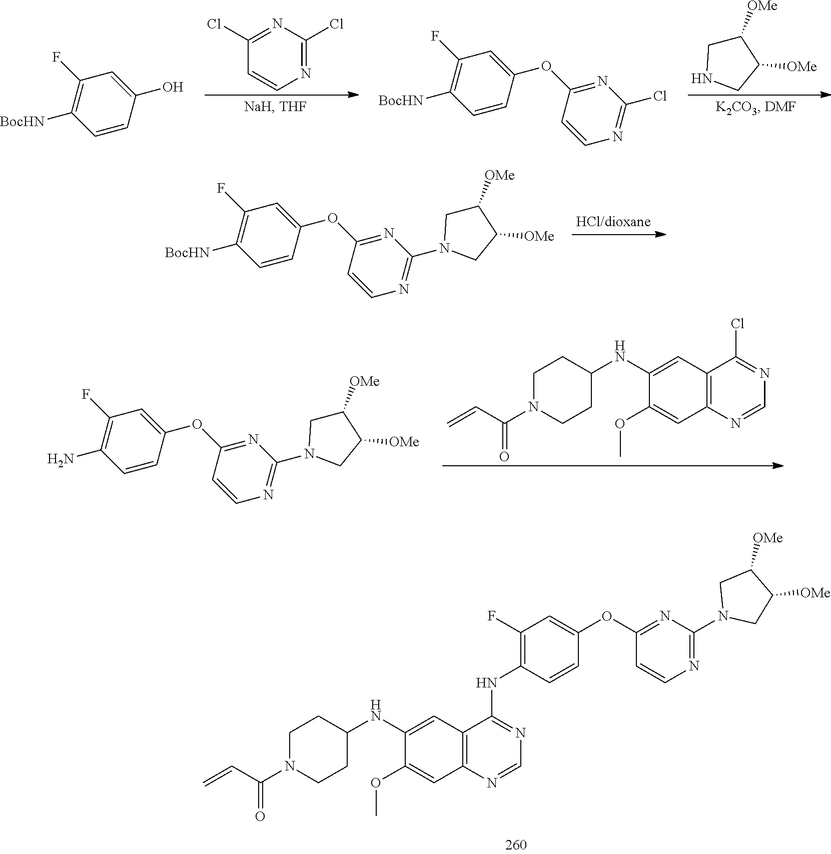

### Step 1: Preparation of tert-butyl (4-((2-chloropyrimidin-4-yl)oxy)-2-fluorophenyl)carbamate

To a solution of tert-butyl (2-fluoro-4-hydroxyphenyl)carbamate (200 mg, 0.88 mmol) in THE (5 mL) was added NaH (45 mg, 60%, 1.05 mmol). The mixture was stirred at 0° C. for 0.5 hours followed by addition of 2,4-dichloropyrimidine (131 mg, 0.88 mmol). The reaction mixture was stirred at room temperature for 15 hours and then diluted with H₂O (20 mL), extracted with EtOAc (20 mL×3). The combined organic layers were washed with water, dried over Na₂SO₄, and concentrated in vacuum to give a crude product. The crude product was purified by Pre-TLC with DCM/MeOH=10:1 to give the desired product (200 mg, 60.1% yield). LC/MS: 340.8[M+H]⁺.

### Step 2: Preparation of tert-butyl (4-((2-((3R,4S)-3,4-dimethoxypyrrolidin-1-yl)pyrimidin-4-yl)oxy)-2-fluorophenyl)carbamate

To a solution of tert-butyl (4-((2-chloropyrimidin-4-yl)oxy)-2-fluorophenyl)carbamate (200 mg, 0.58 mmol) in DMF (5 mL) was added (3R,4S)-3,4-dimethoxypyrrolidine hydrochloride (98 mg, 0.58 mmol) and K₂CO₃ (244 mg, 1.76 mmol) under argon. The reaction mixture was stirred at 110° C. for 15 hours. The mixture was cooled to room temperature, diluted with H₂O (15 mL) and extracted with EtOAc (10 mL×3). The organic phase was concentrated in vacuum to give a crude product. The crude product was purified by Pre-TLC with EA/PE=1:1 to give the desired product (80 mg, 28.1% yield). LC/MS: 435.9[M+H]⁺.

### Step 3: Preparation of 4-((2-((3R,4S)-3,4-dimethoxypyrrolidin-1-yl)pyrimidin-4-yl)oxy)-2-fluoroaniline

To a solution of tert-butyl (4-((2-((3R,4S)-3,4-dimethoxypyrrolidin-1-yl)pyrimidin-4-yl)oxy)-2-fluorophenyl)carbamate (70 mg, 0.16 mmol) in DCM (3 mL) was added HCl/dioxane (4N, 3 mL). The reaction was stirred at room temperature for 1 hour. The solvent was removed in vacuum and the residue was dissolved in saturated NaHCO₃ solution, extracted with DCM. The organic phase was dried over Na₂SO₄ and concentrated in vacuum to give the title compound. (50 mg, 78.9% yield). LC/MS: 334.9 [M+H]⁺.

### Step 4: Preparation of 1-(4-((4-((4-((2-((3R,4S)-3,4-dimethoxypyrrolidin-1-yl)pyrimidin-4-yl)oxy)-2-fluorophenyl)amino)-7-methoxyquinazolin-6-yl)amino)piperidin-1-yl)prop-2-en-1-one (260)

To a solution of 4-((2-((3R,4S)-3,4-dimethoxypyrrolidin-1-yl)pyrimidin-4-yl)oxy)-2-fluoro aniline (40 mg, 0.12 mmol) in MeCN (5 mL) was added 1-(4-((4-chloro-7-methoxyquinazolin-6-yl)amino)piperidin-1-yl)prop-2-en-1-one (42 mg, 0.12 mmol) and TsOH (62 mg, 0.36 mmol) under argon. The mixture was stirred at 80° C. for 3 hours. The reaction mixture was cooled to room temperature, basified with triethylamine, diluted with H₂O (50 mL) and extracted with EtOAc (50 mL×3). The organic phase was concentrated under vacuum. The crude product was purified by Pre-TLC with DCM/MeOH=10:1 to give the desired product (20 mg, 23.2% yield). LC/MS: 644.8 [M+H]⁺. 1H NMR (400 MHz, DMSO) b 9.20 (s, 1H), 8.27 (d, J=5.5 Hz, 1H), 8.21 (s, 1H), 7.57 (t, J=8.8 Hz, 1H), 7.32 (dd, J=11.0, 2.6 Hz, 1H), 7.28 (s, 1H), 7.13 (dd, J=8.6, 1.7 Hz, 1H), 7.08 (s, 1H), 6.86 (dd, J=16.7, 10.5 Hz, 1H), 6.24 (d, J=5.5 Hz, 1H), 6.12 (dd, J=16.7, 2.5 Hz, 1H), 5.69 (dd, J=10.5, 2.5 Hz, 1H), 5.32 (d, J=8.5 Hz, 1H), 4.46 (d, J=11.4 Hz, 1H), 4.14 (d, J=12.6 Hz, 1H), 4.10-3.94 (m, 5H), 3.81-3.78 (m, 1H), 3.61-3.57 (m, 1H), 3.55-3.46 (m, 1H), 3.42-3.35 (m, 1H), 3.32 (s, 6H), 3.24 (t, J=11.9 Hz, 2H), 2.86 (t, J=12.2 Hz, 1H), 2.12-2.03 (m, 2H), 1.57-1.39 (m, 2H).

Compounds in the following table are prepared with procedures described for the above examples.

| Ex- | | |
|---|---|---|
| am- | | |
| ple | | |
| # | Structure | Analytical Data |
| 17 | 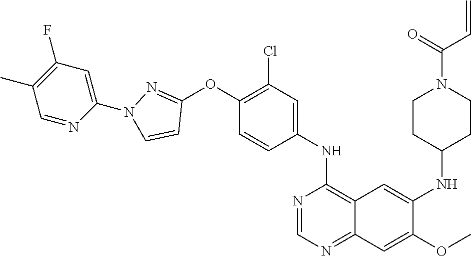 [SMILES: C=CC(=O)N1CCC(Nc2cc3c(Nc4ccc(Oc5ccn(-c6cc(F)c(C)cn6)n5)c(Cl)c4)ncnc3cc2OC)CC1] | LC/MS: 629.0 [M + H]⁺. ¹H NMR (400 MHz, CD₃OD) δ 8.47 (d, J = 2.7 Hz, 1H), 8.38 (s, 1H), 8.27 (d, J = 10.1 Hz, 1H), 7.97 (d, J = 2.5 Hz, 1H), 7.69-7.66 (m, 1H), 7.38 (t, J = 8.9 Hz, 2H), 7.31 (s, 1H), 7.08 (s, 1H), 6.85-6.78 (m, 1H), 6.23-6.19 (m, 1H), 6.09 (d, J = 2.7 Hz, 1H), 5.77-5.74(m, 1H), 4.57 (d, J = 14.8 Hz, 1H), 4.19 (d, J = 13.3 Hz, 1H), 4.04 (s, 3H), 3.87 (d, J = 10.5 Hz, 1H), 3.38 (d, J = 12.4 Hz, 1H), 3.03 (t, J = 12.3 Hz, 1H), 2.23 (d, J = 24.7 Hz, 5H), 1.53 (t, J = 10.1 Hz, 2H). |
| 18 | 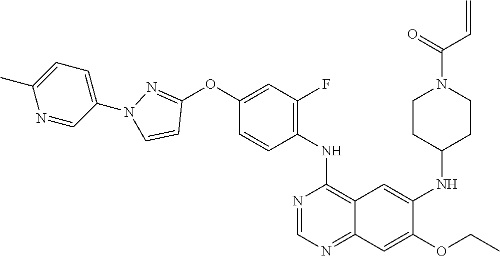 [SMILES: C=CC(=O)N1CCC(Nc2cc3c(Nc4ccc(Oc5ccn(-c6ccc(C)nc6)n5)cc4F)ncnc3cc2OCC)CC1] | LC/MS: 608.7 [M + H]⁺. ¹H NMR (400 MHz, CD₃OD) δ 8.82 (d, J = 2.5 Hz, 1H), 8.29-8.26 (m, 2H), 8.24 (s, 1H), 8.06 (dd, J = 8.5, 2.6 Hz, 1H), 7.55 (t, J = 8.7 Hz, 1H), 7.41 (d, J = 8.5 Hz, 1H), 7.30 (s, 1H), 7.19- 7.10 (m, 2H), 7.06 (s, 1H), 6.81 (dd, J = 16.8, 10.7 Hz, 1H), 6.25-6.17 (m, 2H), 5.75 (dd, J = 10.6, 2.0 Hz, 1H), 4.59 (d, J = 13.4 Hz, 1H), 4.33-4.25 (m, 2H), 4.19 (d, J = 13.8 Hz, 1H), 3.87-3.79 (m, 1H), 3.36 (d, J = 11.9 Hz, 1H), 3.00 (t, J = 11.7 Hz, 1H), 2.58 (s, 3H), 2.28- 2.19 (m, 2H), 1.53 (t, J = 7.0 Hz, 5H). |
| 19 | 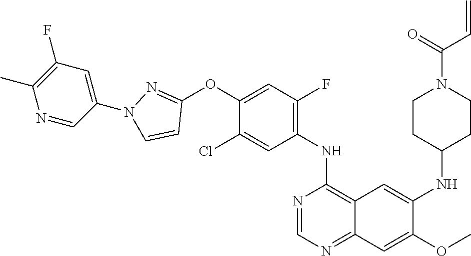 [SMILES: C=CC(=O)N1CCC(Nc2cc3c(Nc4cc(Cl)c(Oc5ccn(-c6cnc(C)c(F)c6)n5)cc4F)ncnc3cc2OC)CC1] | LC/MS: 646.7 [M + H]⁺. 1H NMR (400 MHz, CD₃OD) δ 8.68 (d, J = 2.1 Hz, 1H), 8.31-8.30 (m, 2H), 7.90 (dd, J = 10.4, 2.1 Hz, 1H), 7.81 (d, J = 7.8 Hz, 1H), 7.34 (d, J = 10.7 Hz, 1H), 7.25 (s, 1H), 7.08 (s, 1H), 6.84-6.77 (m, 1H), 6.24-6.18 (m, 2H), 5.75 (dd, J = 10.6, 2.0 Hz, 1H), 4.59-4.56 (m, 1H), 4.19- 4.16 (m, 1H), 4.03 (s, 3H), 3.83-3.81 (m, 1H), 3.32-3.30 (m, 1H), 3.02-2.97 (m, 1H), 2.50 (d, J = 2.9 Hz, 3H), 2.24-2.18 (m, 2H), 1.57-1.49 (m, 2H). |
| 20 | 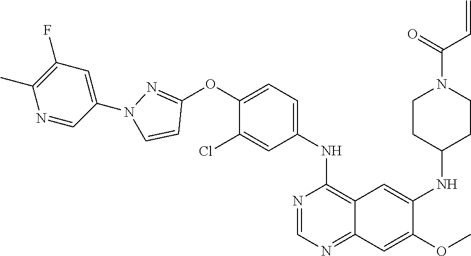 [SMILES: C=CC(=O)N1CCC(Nc2cc3c(Nc4ccc(Oc5ccn(-c6cnc(C)c(F)c6)n5)c(Cl)c4)ncnc3cc2OC)CC1] | LC/MS: 628.7 [M + H]⁺. ¹H NMR (400 MHz, DMSO-d6) δ 9.29 (s, 1H), 8.73 (s, 1H), 8.54 (d, J = 2.7 Hz, 1H), 8.35 (s, 1H), 8.15-8.11 (m, 2H), 8.01-7.99 (m, 1H), 7.81-7.78 (m, 1H), 7.38 (d, J = 8.9 Hz, 1H), 7.25 (s, 1H), 7.07 (s, 1H), 6.86-6.79 (m, 1H), 6.19 (d, J = 2.6 Hz, 1H), 6.11-6.06 (m, 1H), 5.65-5.63 (m, 1H), 5.34 (d, J = 8.7 Hz, 1H), 4.43-4.39 (m, 1H), 4.11-4.09 (m, 1H), 3.94 (s, 3H), 3.85- 3.74 (m, 1H), 3.22-3.19 (m, 1H), 2.89-2.83 (m, 1H), 2.42 (s, 3H), 2.06-2.02 (m, 2H), 1.44-1.41 (m, 2H). |
| 23 | 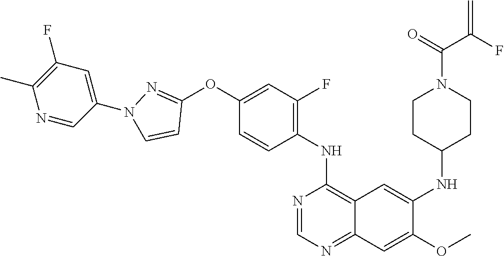 [SMILES: C=C(F)C(=O)N1CCC(Nc2cc3c(Nc4ccc(Oc5ccn(-c6cnc(C)c(F)c6)n5)cc4F)ncnc3cc2OC)CC1] | LC/MS: 631.0 [M + H]⁺. ¹H NMR (400 MHz, MeOD) δ 8.75 (d, J = 2.1 Hz, 1H), 8.36 (d, J = 2.7 Hz, 1H), 8.28 (s, 1H), 7.98 (dd, J = 10.4, 2.2 Hz, 1H), 7.58-7.56 (m, 1H), 7.32 (s, 1H), 7.25-7.14 (m, 2H), 7.11 (s, 1H), 6.28 (d, J = 2.7 Hz, 1H), 5.28-5.25 (m, 1H), 5.23-5.13 (m, 1H), 4.52-4.43 (m, 1H), 4.22-4.11 (m, 1H), 4.07 (s, 3H), 3.90-3.83 (m, 1H), 3.47-3.37 (m, 1H), 3.13-3.02 (m, 1H), 2.54 (d, J = 2.9 Hz, 3H), 2.31-2.22 (m, 2H), 1.64-1.54 (m, 2H). |
| 24 | 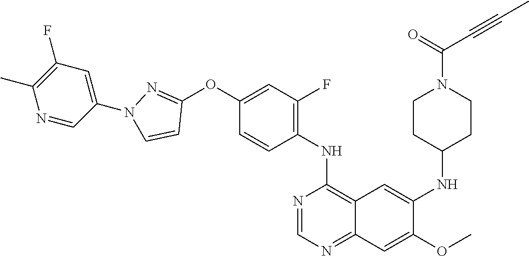 [SMILES: CC#CC(=O)N1CCC(Nc2cc3c(Nc4ccc(Oc5ccn(-c6cnc(C)c(F)c6)n5)cc4F)ncnc3cc2OC)CC1] | LC/MS: 624.7 [M + H]⁺. ¹H NMR (400 MHz, MeOD) δ 8.75 (d, J = 2.1 Hz, 1H), 8.55 (s, 1H), 8.38 (d, J = 2.7 Hz, 1H), 7.99 (dd, J = 10.4, 2.1 Hz, 1H), 7.59 (t, J = 8.7 Hz, 1H), 7.44 (s, 1H), 7.28-7.23 (m, 2.6 Hz, 1H), 7.23-7.18 (m, 1H), 7.15 (s, 1H), 6.30 (d, J = 2.7 Hz, 1H), 4.57-4.46 (m, 2H), 4.14 (s, 3H), 3.91-3.81 (m, 1H), 3.45-3.37 (m, 1H), 3.04-2.95 (m, 1H), 2.55 (d, J = 2.9 Hz, 3H), 2.26-2.17 (m, J = 14.8 Hz, 2H), 2.07 (s, 3H), 1.66-1.58 (m, 1H), 1.55-1.47 (m, 1H). |
| 25 | 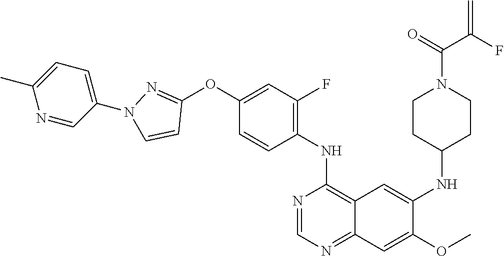 [SMILES: C=C(F)C(=O)N1CCC(Nc2cc3c(Nc4ccc(Oc5ccn(-c6ccc(C)nc6)n5)cc4F)ncnc3cc2OC)CC1] | LC/MS: 612.7 [M + H]⁺. 1H NMR (400 MHz, MeOD) δ 9.02 (d, J = 2.5 Hz, 1H), 8.57-8.49 (m, 2H), 8.45 (d, J = 2.8 Hz, 1H), 7.77 (d, J = 8.7 Hz, 1H), 7.60-7.58 (m, 1H), 7.46 (s, 1H), 7.31-7.19 (m, 2H), 7.17 (s, 1H), 6.36 (d, J = 2.7 Hz, 1H), 5.32-5.26 (m, 1H), 5.19 (dd, J = 28.1, 3.9 Hz, 1H), 4.51-4.47 (m, 1H), 4.16-4.12 (m, 4H), 3.88-3.84 (m, 1H), 3.41-3.37 (m, 1H), 3.09-3.05 (m, 1H), 2.73 (s, 3H), 2.26-2.22 (m, 2H), 1.65-16.1 (m, 2H). |
| 26 | 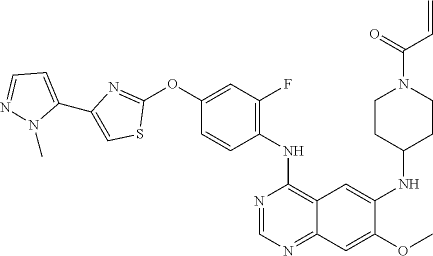 [SMILES: C=CC(=O)N1CCC(Nc2cc3c(Nc4ccc(Oc5nc(-c6ccnn6C)cs5)cc4F)ncnc3cc2OC)CC1] | LC/MS: 600.7 [M + H]⁺. 1H NMR (400 MHz, MeOD) δ 8.33 (s, 1H), 7.73- 7.69 (m, 1H), 7.46-7.41 (m, 3H), 7.35-7.31 (m, 2H), 7.11 (s, 1H), 6.87-6.80 (m, 1H), 6.61 (d, J = 2.0 Hz, 1H), 6.23 (dd, J = 16.8, 2.0 Hz, 1H), 5.77 (dd, J = 10.6, 2.0 Hz, 1H), 4.62-4.59 (m, 1H), 4.25-4.19 (m, 1H), 4.07 (s, 3H), 4.04 (s, 3H), 3.88-3.83 (m, 1H), 3.39-3.37 (m, 1H), 3.04-2.99 (m, 1H), 2.25-2.23 (m, 2H), 1.60- 1.49 (m, 2H). |
| 27 | 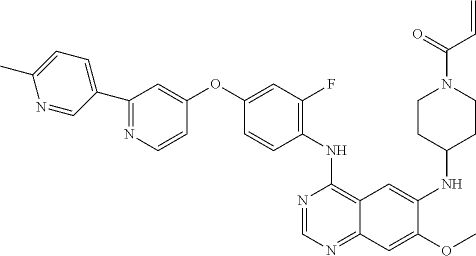 [SMILES: C=CC(=O)N1CCC(Nc2cc3c(Nc4ccc(Oc5ccnc(-c6ccc(C)nc6)c5)cc4F)ncnc3cc2OC)CC1] | LC/MS: 605.8 [M + H]⁺. ¹HNMR (400 MHz, CD₃OD) δ 9.34 (s, 1H), 9.02 (d, J = 8.0 Hz, 1H), 8.70 (d, J = 4.0 Hz, 1H), 8.58 (s, 1H), 7.99 (d, J = 8.0 Hz, 1H), 7.89 (s, 1H), 7.71 (t, J = 4.0 Hz, 1H), 7.48 (s, 1H), 7.33 (d, J = 8.0 Hz, 1H), 7.14-7.25 (m, 3H), 6.85 (dd, J = 12.0, 4.0 Hz, 1H), 6.23 (d, J = 16.0 Hz, 1H), 5.80 (d, J = 8.0 Hz, 1H), 4.65 (d, J = 16.0 Hz, 1H), 4.26 (br, 1H), 4.15 (s, 3H), 3.89 (m, 1H), 3.35 (m, 1H), 3.05 (m, 1H), 2.85 (s, 3H), 2.25 (m, 2H), 1.63 (m, 2H). |
| 28 | 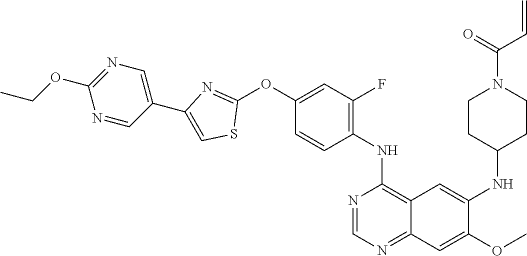 [SMILES: C=CC(=O)N1CCC(Nc2cc3c(Nc4ccc(Oc5nc(-c6cnc(OCC)nc6)cs5)cc4F)ncnc3cc2OC)CC1] | LC/MS: 642.7 [M + H]⁺. 1H NMR (400 MHz, MeOD) δ 8.97 (s, 2H), 8.32 (s, 1H), 7.74-7.70 (m, 1H), 7.54 (s, 1H), 7.43 (dd, J = 10.7, 2.6 Hz, 1H), 7.37-7.31 (m, 2H), 7.10 (s, 1H), 6.87-6.80 (m, 1H), 6.23 (dd, J = 16.8, 2.0 Hz, 1H), 5.77 (dd, J = 10.6, 2.0 Hz, 1H), 4.62-4.59 (m, 1H), 4.49 (q, J = 7.1 Hz, 2H), 4.23-4.19 (m, 1H),4.02 (s, 3H), 3.88- 3.83 (m, 1H), 3.40-3.37 (m, 1H), 3.05-2.99 (m, 1H), 2.26-2.23 (m, 2H), 1.60-1.49 (m, 2H), 1.44 (t, J = 7.1 Hz, 3H). |
| 29 | 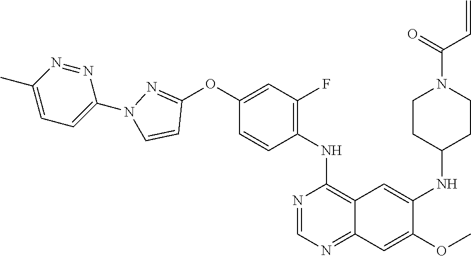 [SMILES: C=CC(=O)N1CCC(Nc2cc3c(Nc4ccc(Oc5ccn(-c6ccc(C)nn6)n5)cc4F)ncnc3cc2OC)CC1] | LC/MS: 595.6 [M + H]⁺. ¹H NMR (400 MHz) δ 9.13 (s, 1H), 8.77 (d, J = 2.8 Hz, 1H), 8.17 (s, 1H), 8.12 (s, 1H), 7.92 (d, J = 9.0 Hz, 1H), 7.73 (d, J = 9.1 Hz, 1H), 7.53- 7.48 (m, 1H), 7.29-7.26 (m, 1H), 7.22 (s, 1H), 7.14-7.08 (m, 1H), 7.04 (s, 1H), 6.86-6.79 (m, 1H), 6.39 (d, J = 2.8 Hz, 1H), 6.10-6.06 (m, 1H), 5.66-5.63 (m, 1H), 5.28 (d, J = 8.5 Hz, 1H), 4.44-4.40 (m, 1H), 4.11-4.09 (m, 1H), 3.93 (s, 3H), 3.72-3.68 (m, 1H), 3.23-3.15 (m, 1H), 2.85-2.79 (m, 1H), 2.62 (s, 3H), 2.04-2.02 (m, 2H), 1.49-1.33 (m, 2H). |
| 30 | 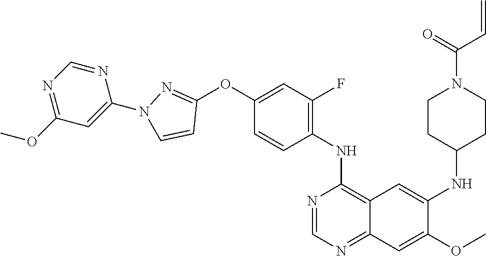 [SMILES: C=CC(=O)N1CCC(Nc2cc3c(Nc4ccc(Oc5ccn(-c6cc(OC)ncn6)n5)cc4F)ncnc3cc2OC)CC1] | LC/MS: 611.7 [M + H]⁺. ¹H NMR (400 MHz, MeOD) δ 8.62-8.54 (m, 2H), 8.27 (s, 1H, FA), 8.25-8.12 (m, 1H), 7.56 (t, J = 8.7 Hz, 1H), 7.29 (d, J = 2.4 Hz, 1H), 7.24-7.11 (m, 2H), 7.09-6.99 (m, 2H), 6.79 (dd, J = 16.8, 10.7 Hz, 1H), 6.29-6.14 (m, 2H), 5.78- 5.68 (m, 1H), 4.63-4.48 (m, 1H), 4.23-4.11 (m, 1H), 4.07 (s, 3H), 4.00 (s, 3H), 3.87-3.74 (m, 1H), 3.40-3.31 (m, 1H), 2.98 (t, J = 12.3 Hz, 1H), 2.28-2.10 (m, 2H), 1.59-1.40 (m, 2H). |
| 32 | 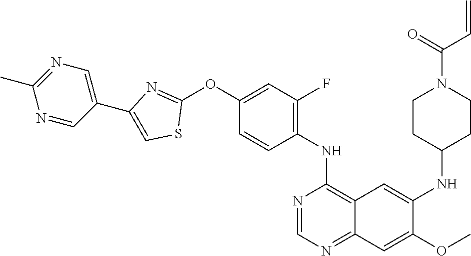 [SMILES: C=CC(=O)N1CCC(Nc2cc3c(Nc4ccc(Oc5nc(-c6cnc(C)nc6)cs5)cc4F)ncnc3cc2OC)CC1] | LC/MS: 612.7 [M + H]+. ¹H NMR (400 MHz, MeOD) δ 9.09 (s, 2H), 8.31 (s, 1H), 7.70 (dd, J = 10.4, 6.9 Hz, 2H), 7.43 (dd, J = 10.7, 2.5 Hz, 1H), 7.33 (d, J = 9.6 Hz, 2H), 7.09 (s, 1H), 6.82 (dd, J = 16.8, 10.7 Hz, 1H), 6.21 (dd, J = 16.8, 1.9 Hz, 1H), 5.75 (dd, J = 10.7, 1.9 Hz, 1H), 4.59 (d, J = 13.9 Hz, 1H), 4.19 (d, J = 13.6 Hz, 1H), 4.05 (s, 3H), 3.89- 3.78 (m, 1H), 3.40-3.32 (m, 1H), 3.00 (t, J = 12.6 Hz, 1H), 2.69 (s, 3H), 2.27-2.17 (m, 2H), 1.59-1.45 (m, 2H). |
| 33 | 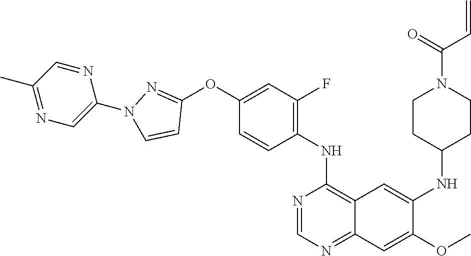 [SMILES: C=CC(=O)N1CCC(Nc2cc3c(Nc4ccc(Oc5ccn(-c6cnc(C)cn6)n5)cc4F)ncnc3cc2OC)CC1] | LC/MS: 596.0 [M + H]⁺. ¹H NMR (400 MHz, MeOD) δ 8.91 (d, J = 1.4 Hz, 1H), 8.51 (d, J = 2.8 Hz, 1H), 8.33 (d, J = 0.7 Hz, 1H), 8.21 (s, 1H), 7.55 (t, J = 8.7 Hz, 1H), 7.27 (s, 1H), 7.19-7.16 (m, 1H), 7.15- 7.12 (m, 1H), 7.06 (s, 1H), 6.79-6.74 (m, 1H), 6.25 (d, J = 2.8 Hz, 1H), 6.18-6.10 (m, 1H), 5.73-5.68 (m, 1H), 4.55 (d, J = 13.6 Hz, 1H), 4.16 (d, J = 12.9 Hz, 1H), 4.02 (s, 3H), 3.85- 3.78 (m, 1H), 3.37-3.32 (m, 1H), 3.02-2.97 (m, 1H), 2.55 (s, 3H), 2.22-2.18 (m, 2H), 1.55- 1.46 (m, 2H). |
| 36 | 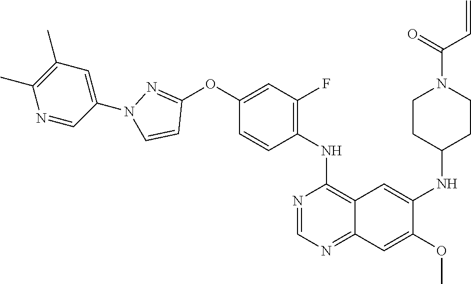 [SMILES: C=CC(=O)N1CCC(Nc2cc3c(Nc4ccc(Oc5ccn(-c6cnc(C)c(C)c6)n5)cc4F)ncnc3cc2OC)CC1] | LC/MS: 608.8 [M + H]+. ¹H NMR (400 MHz, MeOD) δ 8.63 (d, J = 7.9 Hz, 1H), 8.31-8.24 (m, 2H), 7.95 (d, J = 2.1 Hz, 1H), 7.59-7.54 (m, 1H), 7.30 (s, 1H), 7.21-7.07 (m, 3H), 6.83 (dd, J = 16.8, 10.7 Hz, 1H), 6.29- 6.18 (m, 2H), 5.77 (dd, J = 10.6, 1.9 Hz, 1H), 4.61-4.58 (m, 1H), 4.21-4.18 (m, 1H), 4.06 (s, 3H), 3.87-3.81 (m, 1H), 3.38-3.34 (m, 1H), 3.04-2.98 (m, 1H), 2.53 (s, 3H), 2.40 (s, 3H), 2.26-2.20 (m, 2H), 1.58-1.51 (m, 2H). |
| 37 | 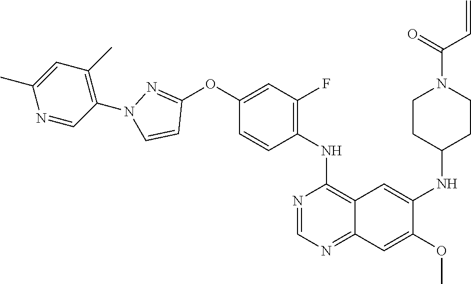 [SMILES: C=CC(=O)N1CCC(Nc2cc3c(Nc4ccc(Oc5ccn(-c6cnc(C)cc6C)n5)cc4F)ncnc3cc2OC)CC1] | LC/MS: 608.7 [M + H]⁺. ¹H NMR (400 MHz, MeOD) δ 8.70 (s, 1H), 8.53 (s, 1H), 8.04 (d, J = 2.0 Hz, 1H), 7.71 (s, 1H), 7.58 (t, J = 8.6 Hz, 1H), 7.44 (s, 1H), 7.20 (m, 3H), 6.84 (m, 1H), 6.34 (d, J = 2.0 Hz, 1H), 6.24 (d, J = 16.7 Hz, 1H), 5.78 (d, J = 10.6 Hz, 1H), 4.62 (d, J = 12.6 Hz, 1H), 4.23 (d, J = 13.5 Hz, 1H), 4.14 (s, 3H), 3.86-3.84 (m, 1H), 3.40-3.38 (m, 1H), 3.01-2.96 (m, 1H), 2.68 (s, 3H), 2.54 (s, 3H), 2.24-2.21 (m, 2H), 1.59-1.56 (m, 2H). |
| 38 | 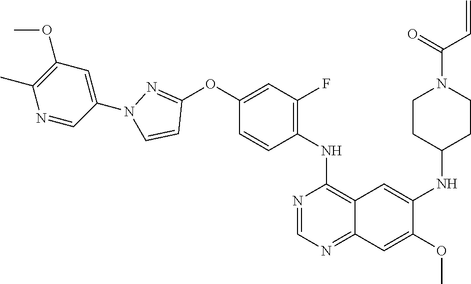 [SMILES: C=CC(=O)N1CCC(Nc2cc3c(Nc4ccc(Oc5ccn(-c6cnc(C)c(OC)c6)n5)cc4F)ncnc3cc2OC)CC1] | LC/MS: 624.8 [M + H]⁺. ¹H NMR (400 MHz, MeOD) δ 8.40 (d, J = 2.1 Hz, 1H), 8.35 (d, J = 2.7 Hz, 1H), 8.26 (s, 1H), 7.72 (d, J = 2.1 Hz, 1H), 7.60-7.55 (m, 1H), 7.31 (s, 1H), 7.22-7.07 (m, 3H), 6.83 (dd, J = 16.8, 10.7 Hz, 1H), 6.28-6.19 (m, 2H), 5.77 (dd, J = 10.7, 2.0 Hz, 1H), 4.61-4.58 (m, 1H), 4.21- 4.18 (m, 1H), 4.06 (s, 3H), 3.99 (s, 3H) 3.87- 3.82 (m, 1H), 3.40-3.37 (m, 1H), 3.05-2.98 (m, 1H), 2.46 (s, 3H), 2.26-2.20 (m, 2H), 1.57-1.51 (m, 2H). |
| 39 | 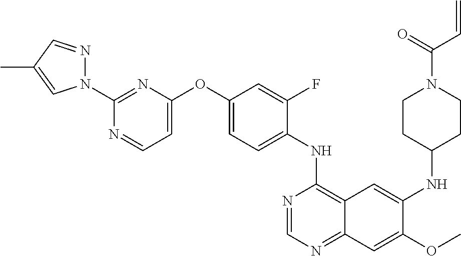 [SMILES: C=CC(=O)N1CCC(Nc2cc3c(Nc4ccc(Oc5ccnc(-n6cc(C)cn6)n5)cc4F)ncnc3cc2OC)CC1] | LC/MS: 595.8 [M + H]⁺. ¹H NMR (400 MHz, MeOD) δ 8.58 (d, J = 8.0 Hz, 1H), 8.50 (s, 1H), 8.16 (s, 1H), 7.57-7.66 (m, 3H), 7.43 (s, 1H), 7.29 (m, 1H), 7.20 (m, 1H), 6.81 (dd, J = 16.0, 4.0 Hz, 1H), 6.20 (m, 2H), 5.75 (d, J = 8.0 Hz, 1H), 4.58 (br, 1H), 4.19 (m, 1H), 3.83 (m, 1H), 3.30 (m, 1H), 2.96 (m, 1H), 2.21 (m, 2H), 2.10 (s, 3H), 1.55 (m, 2H). |
| 42 | 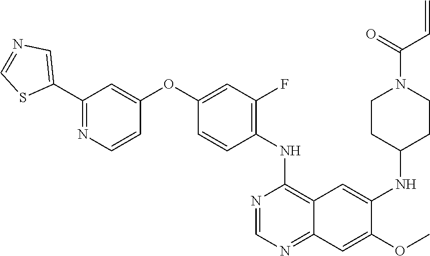 [SMILES: C=CC(=O)N1CCC(Nc2cc3c(Nc4ccc(Oc5ccnc(-c6cncs6)c5)cc4F)ncnc3cc2OC)CC1] | LC/MS: 597.7 [M + H]⁺. ¹H NMR (400 MHz, MeOD) δ 9.08 (s, 1H), 8.48- 8.51 (m, 3H), 8.27 (s, 1H), 7.63-7.69 (m, 2H), 7.32 (s, 1H), 7.17 (m, 2H), 7.13 (s, 1H), 7.04 (m, 1H), 6.85 (dd, J = 16.0, 12.0 Hz, 1H), 6.25 (d, J = 16 Hz, 2H), 5.79 (d, J = 12 Hz, 1H), 4.63 (br, 1H), 4.20 (m, 1H), 4.07 (m, 3H), 3.83 (m, 1H), 3.34 (m, 1H), 3.05 (m, 1H), 2.25 (m, 2H), 1.54 (m, 2H). |
| 43 | 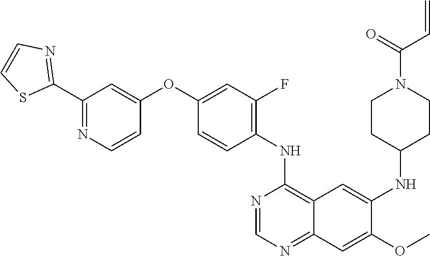 [SMILES: C=CC(=O)N1CCC(Nc2cc3c(Nc4ccc(Oc5ccnc(-c6nccs6)c5)cc4F)ncnc3cc2OC)CC1] | LC/MS: 597.7 [M + H]+ 1H NMR (400 MHz, MeOD) δ 8.52 (d, J = 5.6 Hz, 1H), 8.38 (s, 1H), 8.24 (s, 1H, FA), 7.93 (d, J = 3.1 Hz, 1H), 7.81 (d, J = 2.2 Hz, 1H), 7.73- 7.70 (m, 2H), 7.38 (s, 1H), 7.24 (dd, J = 10.6, 2.6 Hz, 1H), 7.20-7.09 (m, 3H), 6.84 (dd, J = 16.8, 10.7 Hz, 1H), 6.24 (dd, J = 16.8, 1.9 Hz, 1H), 5.78 (dd, J = 10.7, 2.0 Hz, 1H), 4.63-4.60 (m, 1H), 4.24-4.21 (m, 1H), 4.09 (s, 3H), 3.93- 3.84 (m, 1H), 3.40-3.38 (m, 1H), 3.06-3.00 (m, 1H), 2.27-2.23 (m, 2H), 1.62-1.53 (m, 2H). |
| 45 | 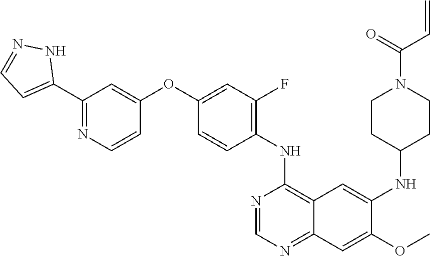 [SMILES: C=CC(=O)N1CCC(Nc2cc3c(Nc4ccc(Oc5ccnc(-c6ccn[nH]6)c5)cc4F)ncnc3cc2OC)CC1] | LC/MS: 581.0 [M + H]+ 1H NMR (400 MHz, MeOD) δ 8.45 (d, J = 4.0 Hz, 1H), 8.30 (s, 1H) 7.59-7.69 (m, 3H), 7.32 (s, 1H), 7.16 (m, 1H), 7.08 (m, 2H), 6.99 (m, 1H), 6.76-6.90 (m, 2H), 6.21 (d, J = 16.0 Hz, 1H), 5.75 (d, J = 12.0 Hz, 1H), 4.58 (d, J = 12.0 Hz, 1H), 4.18 (d, J = 12.0 Hz, 1H), 4.04 (s, 3H), 3.84 (m, 1H), 3.28 (m, 1H), 3.00 (t, J = 12.0 Hz, 1H), 2.20 (d, J = 12.0 Hz, 1H), 1.54 (m, 2H). |
| 47 | 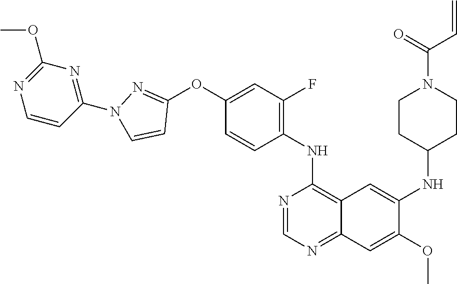 [SMILES: C=CC(=O)N1CCC(Nc2cc3c(Nc4ccc(Oc5ccn(-c6ccnc(OC)n6)n5)cc4F)ncnc3cc2OC)CC1] | LC/MS: 611.7 [M + H]+. ¹H NMR (400 MHz, MeOD) δ 8.64-8.60 (m, 1H), 8.52 (d, J = 5.3 Hz, 1H), 8.29 (s, 1H), 7.56 (t, J = 8.7 Hz, 1H), 7.39-7.34 (m, 1H), 7.30 (s, 1H), 7.23 (dd, J = 11.0, 2.7 Hz, 1H), 7.19-7.13 (m, 1H), 7.08 (s, 1H), 6.79 (dd, J = 16.8, 10.7 Hz, 1H), 6.33-6.27 (m, 1H), 6.23-6.13 (m, 1H), 5.77-5.69 (m, 1H), 4.56 (d, J = 12.3 Hz, 1H), 4.24-4.11 (m, 1H), 4.07-4.01 (m, 6H), 3.81 (t, J = 10.1 Hz, 1H), 3.37-3.31 (m, 1H), 2.98 (t, J = 12.0 Hz, 1H), 2.25-2.14 (m, 2H), 1.59-1.40 (m, 2H). |
| 48 | 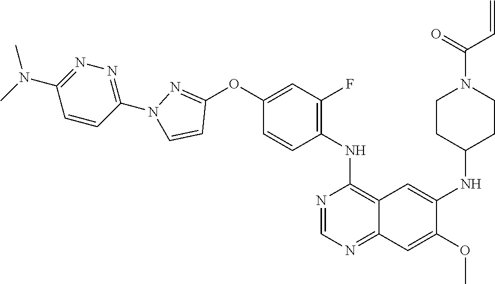 [SMILES: C=CC(=O)N1CCC(Nc2cc3c(Nc4ccc(Oc5ccn(-c6ccc(N(C)C)nn6)n5)cc4F)ncnc3cc2OC)CC1] | LC/MS: 625.1 [M + H]⁺. ¹H NMR (400 MHz, CD₃OD) δ 8.55 (s, 1H), 8.50 (d, J = 2.8 Hz, 1H), 8.24 (d, J = 10.1 Hz, 1H), 7.83 (d, J = 10.1 Hz, 1H), 7.60 (t, J = 8.7 Hz, 1H), 7.45 (s, 1H), 7.30-7.26 (m, 1H), 7.23- 7.20 (m, 1H), 7.17 (s, 1H), 6.88-6.81 (m, 1H), 6.37 (d, J = 2.8 Hz, 1H), 6.26-7.21 (m, 1H), 5.80-5.77 (m, 1H), 4.63 (d, J = 14.2 Hz, 1H), 4.24 (d, J = 13.3 Hz, 1H), 4.14 (s, 3H), 3.86- 3.84 (m, 1H), 3.39-3.35 (m, 7H), 3.00 (t, J = 12.3 Hz, 1H), 2.25-2.21 (m, 2H), 1.62-1.55 (m, 2H). |
| 50 | 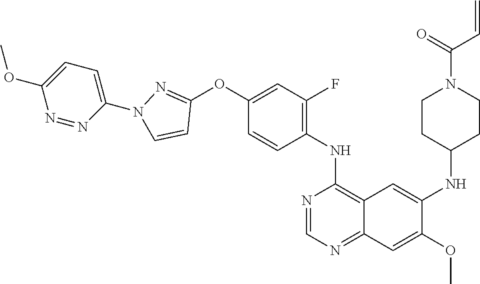 [SMILES: C=CC(=O)N1CCC(Nc2cc3c(Nc4ccc(Oc5ccn(-c6ccc(OC)nn6)n5)cc4F)ncnc3cc2OC)CC1] | LC/MS: 612.0 [M + H]⁺. ¹H NMR (400 MHz, CD₃OD) δ 8.57 (d, J = 2.7 Hz, 1H), 8.24 (d, J = 10.4 Hz, 2H), 8.03 (d, J = 9.4 Hz, 1H), 7.54 (t, J = 8.7 Hz, 1H), 7.30 (d, J = 10.7 Hz, 2H), 7.20-7.7 (m, 1H), 7.13 (d, J = 8.8 Hz, 1H), 7.06 (s, 1H), 6.83-6.76 (m, 1H), 6.27 (d, J = 2.7 Hz, 1H), 6.21-6.16 (m, 1H), 5.75-5.72 (m, 1H), 4.58-4.54 (m, 1H), 4.19-4.16 (m, 1H), 4.09 (s, 3H), 4.03 (s, 3H), 3.85-3.77 (m, 1H), 2.98 (t, J = 11.7 Hz, 1H), 2.19-2.17 (m, 2H), 1.55-1.45 (m, 2H). |
| 51 | 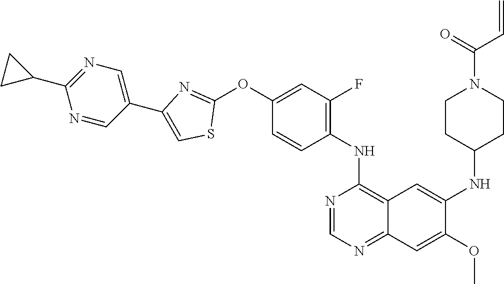 [SMILES: C=CC(=O)N1CCC(Nc2cc3c(Nc4ccc(Oc5nc(-c6cnc(C7CC7)nc6)cs5)cc4F)ncnc3cc2OC)CC1] | LC/MS: 638.7 [M + H]⁺. 1H NMR (400 MHz, MeOD) δ 9.01 (s, 2H), 8.29 (s, 1H), 7.74-7.70 (m, 1H), 7.64 (s, 1H), 7.43 (dd, J = 10.7, 2.6 Hz, 1H), 7.37-7.29 (m, 2H), 7.10 (s, 1H), 6.87-6.80 (m, 1H), 6.23 (dd, J = 16.8, 1.9 Hz, 1H), 5.77 (dd, J = 10.6, 1.9 Hz, 1H), 4.62-4.59 (m, 1H), 4.22-4.20 (m, 1H), 4.06 (s, 3H), 3.88-3.82 (m, 1H), 3.39-3.34 (m, 1H), 3.05-2.99 (m, 1H), 2.29-2.23 (m, 3H), 1.59-1.51 (m, 2H), 1.16-1.10 (m, 4H). |
| 53 | 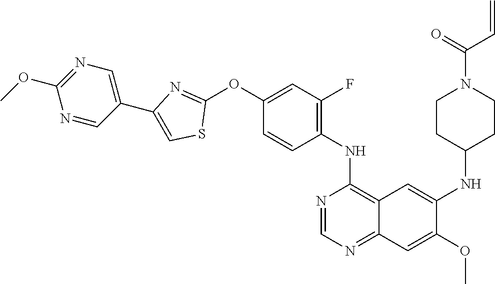 [SMILES: C=CC(=O)N1CCC(Nc2cc3c(Nc4ccc(Oc5nc(-c6cnc(OC)nc6)cs5)cc4F)ncnc3cc2OC)CC1] | LC/MS: 628.7 [M + H]⁺. ¹H NMR (400 MHz, CD3OD) δ 10.12 (s, 2H), 9.70 (s, 1H), 8.83 (t, J = 8.5 Hz, 1H), 8.73 (s, 1H), 8.58 (m, 3H), 8.29 (s, 1H), 7.97 (dd, J = 16.7, 10.7 Hz, 1H), 7.36 (d, J = 16.7 Hz, 1H), 6.91 (d, J = 10.6 Hz, 1H), 5.76 (d, J = 12.7 Hz, 1H), 5.37 (d, J = 13.3 Hz, 1H), 5.27 (s, 3H), 5.19 (s, 3H), 4.99 (t, J = 10.1 Hz, 1H), 4.52-4.50 (m, 1H), 4.13 (t, J = 12.1 Hz, 1H), 3.38-3.33 (m, 2H), 2.70-2.72 (m, 2H). |
| 54 | 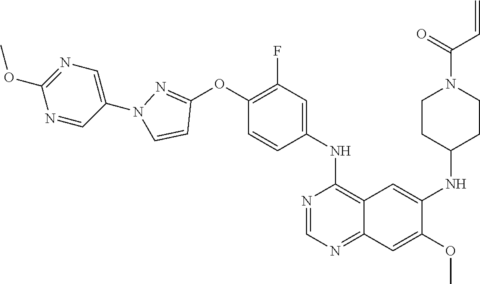 [SMILES: C=CC(=O)N1CCC(Nc2cc3c(Nc4ccc(Oc5ccn(-c6cnc(OC)nc6)n5)c(F)c4)ncnc3cc2OC)CC1] | LC/MS: 611.7 [M + H]⁺. ¹HNMR (400 MHz, CD₃OD) δ 8.86 (s, 2H), 8.60- 8.57 (m, 1H), 8.18 (d, J = 2.7 Hz, 1H), 7.76 (dd, J = 12.2, 2.3 Hz, 1H), 7.53-7.40 (m, 3H), 7.11 (s, 1H), 6.80 (dd, J = 16.8, 10.7 Hz, 1H), 6.24-6.12 (m, 2H), 5.74 (dd, J = 10.6, 2.0 Hz, 1H), 4.58 (d, J = 11.1 Hz, 1H), 4.19 (dd, J = 12.5, 5.7 Hz, 1H), 4.09 (s, 3H), 4.04 (s, 3H), 3.95-3.80 (m, 1H), 3.39-3.32 (m, 1H), 2.98 (t, J = 11.9 Hz, 1H), 2.19-2.17 (m, 2H), 1.64-1.43 (m, 2H). |
| 55 | 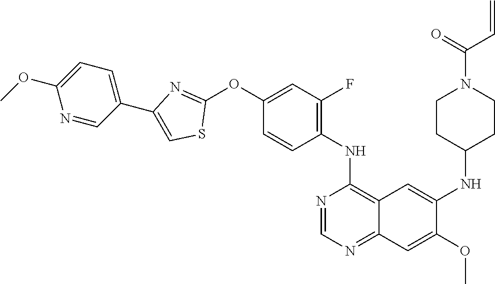 [SMILES: C=CC(=O)N1CCC(Nc2cc3c(Nc4ccc(Oc5nc(-c6ccc(OC)nc6)cs5)cc4F)ncnc3cc2OC)CC1] | LC/MS: 627.7 [M + H]⁺. 1H NMR (400 MHz, CD3OD) δ 8.55 (d, J = 17.9 Hz, 2H), 8.08 (d, J = 8.5 Hz, 1H), 7.65 (t, J = 8.6 Hz, 1H), 7.43-7.46 (m, 2H), 7.34-7.38 (m, 2H), 7.12 (s, 1H), 6.80-6.82 (m, 2H), 6.20 (d, J = 16.7 Hz, 1H), 5.74 (d, J = 10.6 Hz, 1H), 4.59 (d, J = 12.9 Hz, 1H), 4.20 (d, J = 13.3 Hz, 1H), 4.10 (s, 3H), 3.92 (s, 1H), 3.82 (t, J = 10.2 Hz, 1H), 3.35-3.34 (m, 1H), 2.96 (t, J = 12.2 Hz, 1H), 2.21-2.18 (m, 2H), 1.52-1.56 (m, 2H). |
| 56 | 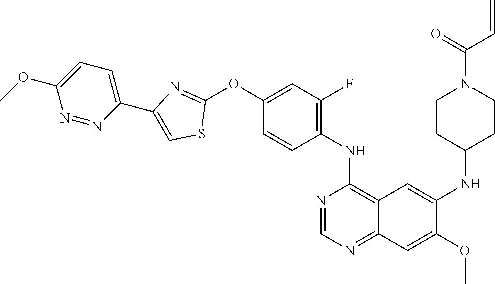 [SMILES: C=CC(=O)N1CCC(Nc2cc3c(Nc4ccc(Oc5nc(-c6ccc(OC)nn6)cs5)cc4F)ncnc3cc2OC)CC1] | LC/MS: 628.9 [M + H]⁺ 1H NMR (400 MHz, MeOD) δ 8.34 (s, 0.5H, FA), 8.29 (s, 1H), 8.09 (d, J = 9.2 Hz, 1H), 7.86 (s, 1H), 7.74-7.71 (m, 1H), 7.45 (dd, J = 10.7, 2.6 Hz, 1H), 7.37-7.30 (m, 2H), 7.25 (d, J = 9.2 Hz, 1H), 7.11 (s, 1H), 6.84 (dd, J = 16.8, 10.7 Hz, 1H), 6.23 (dd, J = 16.8, 2.0 Hz, 1H), 5.78 (dd, J = 10.7, 2.0 Hz, 1H), 4.62-4.59 (m, 1H), 4.23-4.20 (m, 1H), 4.15 (s, 3H), 4.07 (s, 3H), 3.91-3.81 (m, 1H), 3.40-3.37 (m, 1H), 3.06-3.00 (m, 1H), 2.30-2.17 (m, 2H), 1.62- 1.47 (m, 2H). |
| 58 | 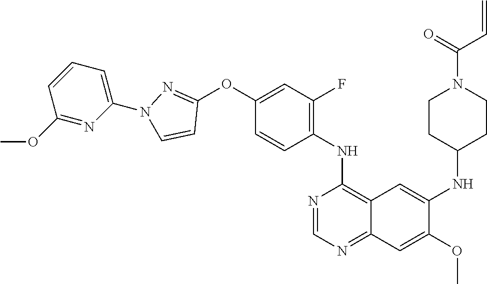 [SMILES: C=CC(=O)N1CCC(Nc2cc3c(Nc4ccc(Oc5ccn(-c6cccc(OC)n6)n5)cc4F)ncnc3cc2OC)CC1] | LC/MS: 611.0 [M + H]⁺. ¹HNMR (400 MHz, CD₃OD) δ 8.54 (d, J = 2.7 Hz, 1H), 8.21 (s, 1H), 7.74 (t, J = 8.0 Hz, 1H), 7.53 (t, J = 8.7 Hz, 1H), 7.31-7.26 (m, 2H), 7.16 (dd, J = 11.1, 2.7 Hz, 1H), 7.11 (ddd, J = 8.8, 2.7, 1.1 Hz, 1H), 7.06 (s, 1H), 6.79 (dd, J = 16.8, 10.7 Hz, 1H), 6.65 (d, J = 8.0 Hz, 1H), 6.21-6.16 (m, 2H), 5.73 (dd, J = 10.7, 1.9 Hz, 1H), 4.56 (d, J = 13.9 Hz, 1H), 4.16 (d, J = 12.9 Hz, 1H), 4.03 (s, 3H), 3.98 (s, 3H), 3.85-3.76 (m, 1H), 3.38-3.30 (m, 1H), 2.98 (t, J = 11.5 Hz, 1H), 2.23-2.15 (m, 2H), 1.56-1.43 (m, 2H). |
| 59 | 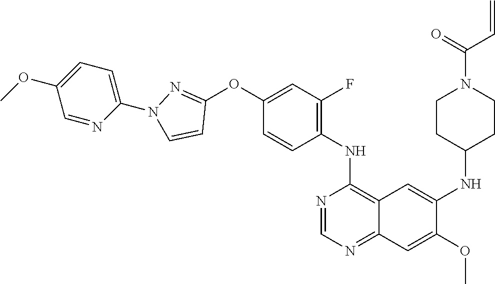 [SMILES: C=CC(=O)N1CCC(Nc2cc3c(Nc4ccc(Oc5ccn(-c6ccc(OC)cn6)n5)cc4F)ncnc3cc2OC)CC1] | LC/MS: 610.9 [M + H]⁺. ¹HNMR (400 MHz, CD₃OD) δ 8.50 (s, 1H), 8.45 (d, J = 4.0 Hz, 1H), 7.72 (d, J = 12.0 Hz, 1H), 7.50 (m, 2H), 7.40 (s, 2H), 7.10-7.17 (m, 3H), 6.80 (dd, J = 16.0, 12.0 Hz, 1H), 6.21 (s,1H), 6.18 (d, J = 4.0 Hz, 1H), 5.74 (dd, J = 12.0, 4.0 Hz, 1H), 4.60 (d, J = 12.0 Hz, 1H), 4.09 (s, 3H), 3.80 (s, 3H), 3.28-3.30 (m, 1H), 2.96 (t, J = 12,0 Hz, 1H), 2.18 (d, J = 12.0 Hz, 2H), 1.51- 1.57 (m, 2H). |
| 86 | 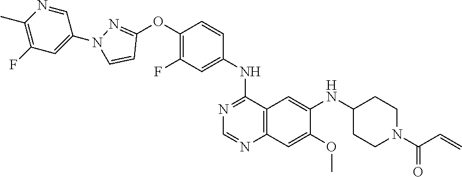 [SMILES: C=CC(=O)N1CCC(Nc2cc3c(Nc4ccc(Oc5ccn(-c6cnc(C)c(F)c6)n5)c(F)c4)ncnc3cc2OC)CC1] | LC/MS: 612.8 [M + H]⁺. ¹H NMR (400 MHz, CD₃OD) δ 8.66 (d, J = 2.0 Hz, 1H), 8.39 (s, 1H), 8.26 (d, J = 2.7 Hz, 1H), 7.89 (dd, J = 10.3, 2.1 Hz, 1H), 7.82 (dd, J = 12.7, 2.4 Hz, 1H), 7.50 (d, J = 8.8 Hz, 1H), 7.39 (t, J = 8.8 Hz, 1H), 7.34-7.28 (m, 1H), 7.11-7.05 (m, 1H), 6.82 (dd, J = 16.8, 10.7 Hz, 1H), 6.25- 6.18 (m, 1H), 6.18-6.11 (m, 1H), 5.76 (dd, J = 10.6, 1.9 Hz, 1H), 4.58 (d, J = 12.8 Hz, 1H), 4.19 (d, J = 13.6 Hz, 1H), 4.04 (s, 3H), 3.94- 3.83 (m, 1H), 3.42-3.34 (m, 1H), 3.03 (t, J = |
| | | 12.2 Hz, 1H), 2.50 (s, 3H), 2.25-2.15 (m, 2H), |
| | | 1.63-1.45 (m, 2H). |
| 88 | 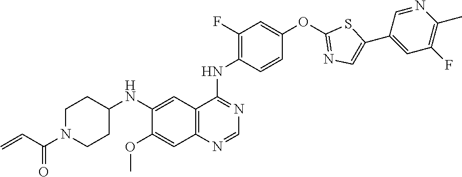 [SMILES: C=CC(=O)N1CCC(Nc2cc3c(Nc4ccc(Oc5ncc(-c6cnc(C)c(F)c6)s5)cc4F)ncnc3cc2OC)CC1] | LC/MS: 630.2 [M + H]⁺. 1H NMR (400 MHz, DMSO) δ 9.48 (s, 1H), 8.56 (t, J = 1.5 Hz, 1H), 8.31 (s, 1H), 8.02 (dd, J = 10.7, 1.9 Hz, 1H), 7.95 (s, 1H), 7.66 (t, J = 8.7 Hz, 1H), 7.59 (dd, J = 10.7, 2.7 Hz, 1H), 7.39- 7.34 (m, 1H), 7.30 (s, 1H), 7.10 (s, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.45 (d, J = 8.5 Hz, 1H), 4.46 (d, J = 12.1 Hz, 1H), 4.14 (d, J = 14.4 Hz, 1H), 3.98 (s, 3H), 3.83- 3.74 (m, 1H), 3.23 (t, J = 11.9 Hz, 1H), 2.86 (t, |
| | | J = 11.7 Hz, 1H), 2.46 (d, J = 2.9 Hz, 3H), 2.10- |
| | | 2.04 (m, 2H), 1.52-1.38 (m, 2H) |
| 89 | 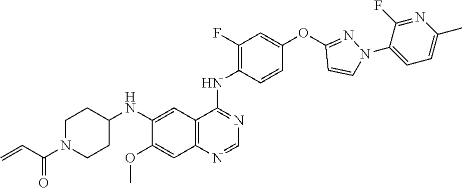 [SMILES: C=CC(=O)N1CCC(Nc2cc3c(Nc4ccc(Oc5ccn(-c6ccc(C)nc6F)n5)cc4F)ncnc3cc2OC)CC1] | LC/MS: 613.2 [M + H]⁺. ¹H NMR (400 MHz, DMSO) δ 9.31 (s, 1H), 8.26- 8.24 (m, 2H), 8.16 (dd, J = 10.1, 7.9 Hz, 1H), 7.52 (t, J = 8.8 Hz, 1H), 7.38 (d, J = 7.6 Hz, 1H), 7.30-7.24 (m, 2H), 7.12 (dd, J = 8.7, 1.8 Hz, 1H), 7.08 (s, 1H), 6.86 (dd, J = 16.7, 10.5 Hz, 1H), 6.35 (d, J = 2.7 Hz, 1H), 6.12 (dd, J = 16.7, 2.5 Hz, 1H), 5.68 (dd, J = 10.4, 2.5 Hz, 1H), 5.38 (d, J = 8.5 Hz, 1H), 4.46 (d, J = 12.2 Hz, 1H), 4.13 (d, J = 12.4 Hz, 1H), 3.97 (s, 3H), 3.76- 3.74 (m, 1H), 3.22 (t, J = 12.1 Hz, 1H), 2.85 (t, J = 11.9 Hz, 1H), 2.48 (s, 3H), 2.08-2.03 (m, |
| | | 2H), 1.50-1.38 (m, 2H). |
| 90 | 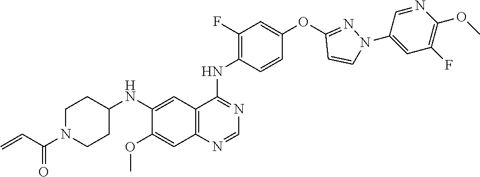 [SMILES: C=CC(=O)N1CCC(Nc2cc3c(Nc4ccc(Oc5ccn(-c6cnc(OC)c(F)c6)n5)cc4F)ncnc3cc2OC)CC1] | LC/MS: 629.2 [M + H]⁺. ¹H NMR (400 MHz, DMSO) δ = 9.16 (s, 1H), 8.51 (d, J = 2.4 Hz, 1H), 8.46 (d, J = 2.4 Hz, 1H), 8.23-8.18 (m, 3H), 7.52 (t, J = 8.8 Hz, 1H), 7.27-7.22 (m, 2H), 7.12-7.08 (m, 1H), 7.07 (s, 1H), 6.86 (dd, J = 16.8, 10.4 Hz, 1H), 6.35 (d, J = 2.4 Hz, 1H), 6.12 (dd, J = 16.8, 2.4 Hz, 1H), 5.68 (dd, J = 10.4, 2.4 Hz, 1H), 5.32 (d, J = 8.8 Hz, 1H), 4.46 (d, J = 12.0 Hz, 1H), 4.13 (d, J = 14.0 Hz, 1H), 3.99 (s, 3H), 3.96 (s, 3H), 3.79-3.71 (m, 1H), 3.22 (t, J = 12.0 Hz, |
| | | 1H), 2.85 (t, J = 12.0 Hz, 1H), 2.12-2.04 (m, |
| | | 2H), 1.48-1.38 (m, 2H). |
| 91 | 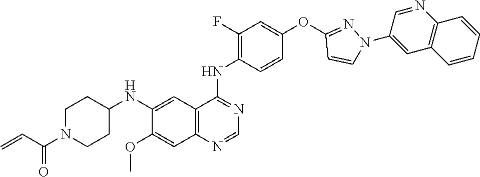 [SMILES: C=CC(=O)N1CCC(Nc2cc3c(Nc4ccc(Oc5ccn(-c6cnc7ccccc7c6)n5)cc4F)ncnc3cc2OC)CC1] | LC/MS: 631.2 [M + H]⁺. ¹H NMR (400 MHz, DMSO) δ 9.45 (d, J = 2.6 Hz, 1H), 9.20 (brs, 1H), 8.79 (d, J = 2.7 Hz, 1H), 8.70 (d, J = 2.6 Hz, 1H), 8.22 (s, 1H), 8.08 (d, J = 9.2 Hz, 2H), 7.80-7.74 (m, 1H), 7.70-7.66 (m, 1H), 7.55 (t, J = 8.8 Hz, 1H), 7.33-7.25 (m, 2H), 7.16 (dd, J = 8.7, 1.9 Hz, 1H), 7.08 (s, 1H), 6.86 (dd, J = 16.7, 10.5 Hz, 1H), 6.45 (d, J = 2.6 Hz, 1H), 6.12 (dd, J = 16.7, 2.4 Hz, 1H), 5.69 (dd, J = 10.4, 2.4 Hz, 1H), 5.34 (d, J = 8.5 Hz, 1H), 4.47 (d, J = 12.3 Hz, 1H), 4.14 (d, J = 12.9 |
| | | Hz, 1H), 3.97 (s, 3H), 3.79-3.73 (m, 1H), 3.26- |
| | | 3.20 (m, 1H), 2.86 (t, J = 12.0 Hz, 1H), 2.09- |
| | | 2.06 (m, 2H), 1.50-1.39 (m, 2H). |
| 92 | 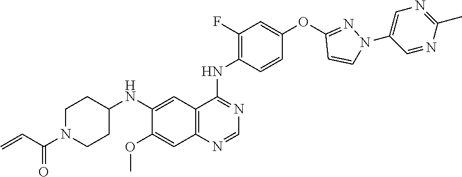 [SMILES: C=CC(=O)N1CCC(Nc2cc3c(Nc4ccc(Oc5ccn(-c6cnc(C)nc6)n5)cc4F)ncnc3cc2OC)CC1] | LC/MS: 596.2 [M + H]⁺. 1H NMR (400 MHz, MeOD) δ 9.08 (s, 2H), 8.36 (d, J = 2.7 Hz, 1H), 8.24 (s, 1H), 7.56 (t, J = 8.6 Hz, 1H), 7.29 (s, 1H), 7.21-7.12 (m, 2H), 7.08 (s, 1H), 6.82 (dd, J = 16.8, 10.6 Hz, 1H), 6.37- 6.13 (m, 3H), 5.75 (dd, J = 10.6, 1.9 Hz, 1H), 4.58 (d, J = 14.0 Hz, 2H), 4.19 (d, J = 13.3 Hz, 1H), 4.04 (s, 3H), 3.87-3.79 (m, 1H), 3.41- 3.34 (m, 1H), 3.04-2.97 (m, 1H), 2.72 (s, 3H), 2.26-2.17 (m, 2H), 1.59-1.47 (m, 2H). |
| 93 | 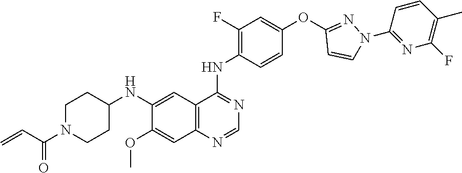 [SMILES: C=CC(=O)N1CCC(Nc2cc3c(Nc4ccc(Oc5ccn(-c6ccc(C)c(F)n6)n5)cc4F)ncnc3cc2OC)CC1] | LC/MS: 613.1 [M + H]⁺. ¹H NMR (400 MHz, DMSO) δ = 9.17 (s, 1H), 8.50 (d, J = 2.8 Hz, 1H), 8.21 (s, 2H), 8.17 (s, 1H), 8.0 (t, J = 8.8 Hz, 1H), 7.58-7.50 (m, 2H), 7.29 (dd, J = 11.2, 2.7 Hz, 1H), 7.25 (s, 1H), 7.15-7.11 (m, 1H), 7.07 (s, 1H), 6.86 (dd, J = 16.8, 10.4 Hz, 1H), 6.34 (d, J = 2.4 Hz, 1H), 6.12 (dd, J = 16.8, 2.4 Hz, 1H), 5.69 (dd, J = 10.4, 2.4 Hz, 1H), 5.33 (d, J = 8.8 Hz, 1H), 4.46 (d, J = 12.4 Hz, 1H), 4.14 (d, J = 12.0 Hz, 1H), 3.96 (s, 3H), 3.78-3.71 (m, 1H), 3.22 (t, J = |
| | | 13.2 Hz, 1H), 2.85 (t, J = 12.0 Hz, 1H), 2.26 (s, |
| | | 3H), 2.11-2.03 (m, 2H), 1.48-1.39 (m, 2H). |
| 94 | 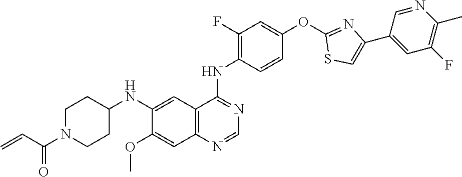 [SMILES: C=CC(=O)N1CCC(Nc2cc3c(Nc4ccc(Oc5nc(-c6cnc(C)c(F)c6)cs5)cc4F)ncnc3cc2OC)CC1] | LC/MS: 629.8 [M + H]⁺. 1H NMR (400 MHz, DMSO) δ 9.25 (s, 1H), 8.55 (s, 1H), 8.24 (s, 1H), 8.02 (dd, J = 10.7, 1.8 Hz, 1H), 7.94 (s, 1H), 7.65 (t, J = 8.8 Hz, 1H), 7.57 (dd, J = 10.7, 2.7 Hz, 1H), 7.34 (dd, J = 8.7, 1.8 Hz, 1H), 7.26 (s, 1H), 7.09 (s, 1H), 6.86 (dd, J = 16.7, 10.5 Hz, 1H), 6.12 (dd, J = 16.7, 2.4 Hz, 1H), 5.68 (dd, J = 10.4, 2.4 Hz, 1H), 5.36 (d, J = 8.5 Hz, 1H), 4.46 (d, J = 11.7 Hz, 1H), 4.13 (d, J = 12.5 Hz, 1H), 3.97 (s, 3H), 3.76 (d, J = 9.0 Hz, 1H), 3.23 (t, J = 12.0 Hz, 1H), 2.86 (t, |
| | | J = 11.6 Hz, 1H), 2.46 (d, J = 2.8 Hz, 3H), 2.09- |
| | | 2.05 (m, 2H), 1.51-1.39 (m, 2H). |
| 95 | 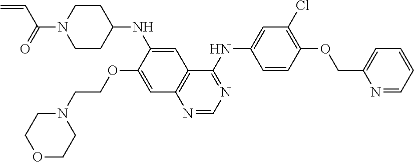 [SMILES: C=CC(=O)N1CCC(Nc2cc3c(Nc4ccc(OCc5ccccn5)c(Cl)c4)ncnc3cc2OCCN2CCOCC2)CC1] | LC/MS: 644.3 [M + H]⁺. ¹H NMR (400 MHz, MeOD) δ 8.64-8.61 (m, 1H), 8.57 (s, 1H), 8.06-8.02 (m, 1H), 7.84- 7.80 (m, 2H), 7.63-7.48 (m, 3H), 7.29-7.21 (m, 2H), 6.83 (dd, J = 16.8, 10.7 Hz, 1H), 6.23 (dd, J = 16.8, 2.0 Hz, 1H), 5.77 (dd, J = 10.6, 2.0 Hz, 1H), 5.36 (s, 2H), 4.75-4.60 (m, 3H), 4.25 (d, J = 12.3 Hz, 1H), 4.05-3.86 (m, 4H), 3.85-3.79 (m, 2H), 3.60-3.40 (m, 4H), 3.35- 3.32 (m, 2H), 2.99-2.99 (m, 1H), 2.18-2.27 (m, 2H), 1.51-1.61 (m, 2H). |
| 96 | 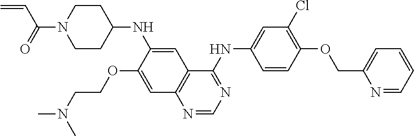 [SMILES: C=CC(=O)N1CCC(Nc2cc3c(Nc4ccc(OCc5ccccn5)c(Cl)c4)ncnc3cc2OCCN(C)C)CC1] | LC/MS: 602.2 [M + H]⁺. ¹H NMR (400 MHz, DMSO) δ 10.77 (brs, 1H), 9.83 (brs, 1H), 8.74 (s, 1H), 8.62 (d, J = 5.0 Hz, 1H), 7.91 (t, J = 7.7 Hz, 1H), 7.81 (d, J = 2.5 Hz, 1H), 7.62-7.48 (m, 3H), 7.45-7.35 (m, 2H), 7.25-7.18 (m, 1H), 6.88 (dd, J = 16.6, 10.2 Hz, 1H), 6.13 (dd, J = 16.3, 2.1 Hz, 1H), 5.88-5.75 (m, 1H), 5.71 (dd, J = 10.5, 2.2 Hz, 1H), 5.36 (s, 2H), 4.60-4.52 (m, 2H), 4.25-4.16 (m, 2H), |
| | | 3.88-3.81 (m, 2H), 3.68 (s, 2H), 3.29-3.21 (m, |
| | | 1H), 2.92 (d, J = 4.5 Hz, 6H), 2.15-2.01 (m, |
| | | 2H), 1.55-1.40 (m, 2H). |
| 98 | 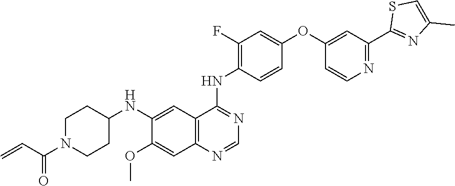 [SMILES: C=CC(=O)N1CCC(Nc2cc3c(Nc4ccc(Oc5ccnc(-c6nc(C)cs6)c5)cc4F)ncnc3cc2OC)CC1] | LC/MS: 612.1 [M + H]⁺. ¹HNMR (400 MHz,) δ 9.75 (brs, 1H), 8.58 (d, J = 5.6 Hz, 1H), 8.42 (s, 1H), 7.70 (t, J = 8.7 Hz, 1H), 7.53-7.43 (m, 3H), 7.33 (s, 1H), 7.26- 7.19 (m, 2H), 7.11 (s, 1H), 6.87 (dd, J = 16.7, 10.5 Hz, 1H), 6.12 (dd, J = 16.7, 2.5 Hz, 1H), 5.69 (dd, J = 10.5, 2.5 Hz, 1H), 5.59 (d, J = 7.6 Hz, 1H), 4.48 (d, J = 13.4 Hz, 1H), 4.15 (d, J = 12.1 Hz, 1H), 4.00 (s, 3H), 3.81-3.72 (m, 1H), 3.27-3.19 (m, 1H), 2.90-2.81 (m, 1H), 2.41 (d, J = 0.9 Hz, 3H), 2.12-2.03 (m, 2H), 1.53- 1.41 (m, 2H). |
| 99 | 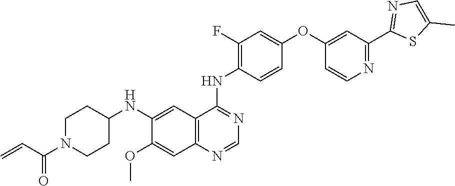 [SMILES: C=CC(=O)N1CCC(Nc2cc3c(Nc4ccc(Oc5ccnc(-c6ncc(C)s6)c5)cc4F)ncnc3cc2OC)CC1] | LC/MS: 613.0 [M + H]⁺. ¹H NMR (400 MHz, MeOD) δ 8.47 (d, J = 5.7 Hz, 1H), 8.35 (s, 1H), 7.73-7.67 (m, 2H), 7.58 (d, J = 1.1 Hz, 1H), 7.35 (s, 1H), 7.21 (dd, J = 10.6, 2.6 Hz, 1H), 7.16-7.12 (m, 2H), 7.09 (dd, J = 5.7, 2.5 Hz, 1H), 6.82 (dd, J = 16.8, 10.7 Hz, 1H), 6.21 (dd, J = 16.8, 2.0 Hz, 1H), 5.76 (dd, J = 10.7, 2.0 Hz, 1H), 4.59 (d, J = 14.2 Hz, 1H), 4.20 (d, J = 13.8 Hz, 1H), 4.06 (s, 3H), 3.86 (t, J = 10.5 Hz, 1H), 3.38 (d, J = 12.5 Hz, 1H), 3.01 (t, J = 11.6 Hz, 1H), 2.54 (d, J = 1.0 Hz, 3H), 2.25-2.20 (m, 2H), 1.58-1.49 (m, 2H). |
| 102 | 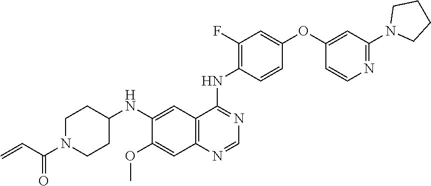 [SMILES: C=CC(=O)N1CCC(Nc2cc3c(Nc4ccc(Oc5ccnc(N6CCCC6)c5)cc4F)ncnc3cc2OC)CC1] | LC/MS: 584.0 [M + H]⁺. ¹H NMR (400 MHz, DMSO) δ 9.50 (s, 1H), 8.30 (s, 1H), 8.02 (d, J = 5.8 Hz, 1H), 7.59 (t, J = 8.8 Hz, 1H), 7.33 (s, 1H), 7.25 (dd, J = 11.0, 2.6 Hz, 1H), 7.11 (s, 1H), 7.10-7.05 (m, 1H), 6.87 (dd, J = 16.7, 10.5 Hz, 1H), 6.23 (dd, J = 5.8, 2.1 Hz, 1H), 6.12 (dd, J = 16.7, 2.5 Hz, 1H), 6.04 (d, J = 2.0 Hz, 1H), 5.69 (dd, J = 10.4, 2.5 Hz, 1H), 5.46 (d, J = 8.4 Hz, 1H), 4.47 (d, J = 12.6 Hz, 1H), 4.14 (d, J = 13.8 Hz, 1H), 3.98 (s, 3H), 3.74- 3.68 (m, 1H), 3.39-3.35 (m, 5H), 2.86 (t, J = 11.8 Hz, 1H), 2.10-2.02 (m, 2H), 1.96-1.90 |
| | | (m, 4H), 1.51-1.36 (m, 2H). |
| 103 | 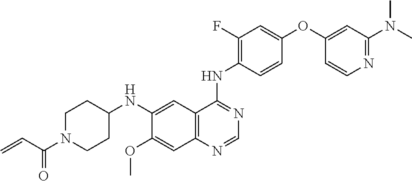 [SMILES: C=CC(=O)N1CCC(Nc2cc3c(Nc4ccc(Oc5ccnc(N(C)C)c5)cc4F)ncnc3cc2OC)CC1] | LC/MS: 558.2 [M + H]⁺. ¹H NMR (400 MHz, DMSO) δ 9.17 (s, 1H), 8.22 (s, 1H), 8.07-8.02 (m, 1H), 7.57 (t, J = 8.8 Hz, 1H), 7.32-7.17 (m, 2H), 7.12-6.98 (m, 2H), 6.86 (dd, J = 16.7, 10.5 Hz, 1H), 6.25-6.05 (m, 3H), 5.69 (dd, J = 10.4, 2.5 Hz, 1H), 5.34 (d, J = 8.5 Hz, 1H), 4.46 (d, J = 12.5 Hz, 1H), 4.14 (d, J = 13.9 Hz, 1H), 3.96 (s, 3H), 3.75 (d, J = 8.2 Hz, 1H), 3.29-3.13 (m, 1H), 3.00 (s, 6H), 2.93-2.79 (m, 1H), 2.15-2.03 (m, 2H), 1.52- 1.34 (m, 2H). |
| 104 | 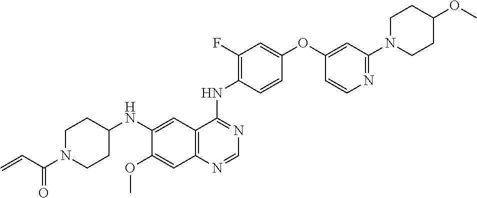 [SMILES: C=CC(=O)N1CCC(Nc2cc3c(Nc4ccc(Oc5ccnc(N6CCC(OC)CC6)c5)cc4F)ncnc3cc2OC)CC1] | LC/MS: 628.3 [M + H]⁺. 1H NMR (400 MHz, DMSO-d6) δ 9.75 (brs, 1H), 8.36 (s, 1H), 8.06 (d, J = 5.7 Hz, 1H), 7.58 (t, J = 8.8 Hz, 1H), 7.37 (s, 1H), 7.24 (dd, J = 11.0, 2.7 Hz, 1H), 7.11 (s, 1H), 7.05 (ddd, J = 8.7, 2.7, 0.9 Hz, 1H), 6.86 (dd, J = 16.7, 10.5 Hz, 1H), 6.46 (d, J = 2.0 Hz, 1H), 6.23 (dd, J = 5.7, 2.0 Hz, 1H), 6.12 (dd, J = 16.7, 2.5 Hz, 1H), 5.69 (dd, J = 10.4, 2.5 Hz, 1H), 5.54 (d, J = 8.4 Hz, 1H), 4.47 (d, J = 13.7 Hz, 1H), 4.14 (d, J = 13.8 Hz, 1H), 3.99 (s, 3H), 3.95-3.86 (m, 2H), 3.82-3.74 (m, 1H), 3.45-3.38 (m, 1H), 3.26 (s, 3H), 3.21-3.10 (m, 3H), 2.89-2.83 (m, 1H), |
| | | 2.10-2.02 (m, 2H), 1.90-1.84 (m, 2H), 1.47- |
| | | 1.35 (m, 4H). |
| 105 | 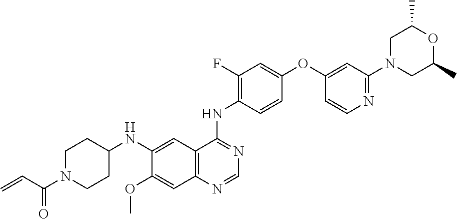 [SMILES: C=CC(=O)N1CCC(Nc2cc3c(Nc4ccc(Oc5ccnc(N6C[C@H](C)O[C@@H](C)C6)c5)cc4F)ncnc3cc2OC)CC1] | LC/MS: 628.2 [M + H]⁺. ¹H NMR (400 MHz, DMSO) δ 9.19 (s, 1H), 8.22 (s, 1H), 8.05 (d, J = 5.6 Hz, 1H), 7.57 (t, J = 8.8 Hz, 1H), 7.26 (s, 1H), 7.21 (dd, J = 11.2, 2.8 Hz, 1H), 7.08 (s, 1H), 7.02 (dd, J = 8.8, 1.6 Hz, 1H), 6.86 (dd, J = 16.8, 10.4 Hz, 1H), 6.46 (d, J = 1.6 Hz, 1H), 6.23 (dd, J = 5.6, 2.0 Hz, 1H), 6.12 (dd, J = 16.8, 2.4 Hz, 1H), 5.69 (dd, J = 10.4, 2.6 Hz, 1H), 5.34 (d, J = 8.4 Hz, 1H), 4.46 (d, J = 13.6 Hz, 1H), 4.14 (d, J = 13.6 Hz, 1H), 4.15-3.95 (m, 5H), 3.78-3.72 (m, 1H), 3.59 (dd, J = 12.8, 3.2 Hz, 2H), 3.29-3.12 (m, 3H), 2.85 (t, J = 12.0 Hz, 1H), 2.10-2.02 (m, 2H), 1.53-1.42 (m, 2H), 1.15 (d, J = 6.4 Hz, 6H). |
| 106 | 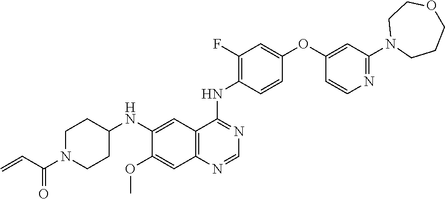 [SMILES: C=CC(=O)N1CCC(Nc2cc3c(Nc4ccc(Oc5ccnc(N6CCCOCC6)c5)cc4F)ncnc3cc2OC)CC1] | LC/MS: 614.2 [M + H]⁺. NMR (400 MHz,) δ 9.53 (s, 1H), 8.31 (s, 1H), 8.04 (d, J = 5.7 Hz, 1H), 7.58 (t, J = 8.8 Hz, 1H), 7.32 (s, 1H), 7.23 (dd, J = 11.0, 2.5 Hz, 1H), 7.10 (s, 1H), 7.05 (dd, J = 8.6, 2.0 Hz, 1H), 6.86 (dd, J = 16.7, 10.5 Hz, 1H), 6.29 (d, J = 1.8 Hz, 1H), 6.19 (dd, J = 5.7, 1.9 Hz, 1H), 6.12 (dd, J = 16.7, 2.4 Hz, 1H), 5.69 (dd, J = 10.4, 2.4 Hz, 1H), 5.47 (d, J = 8.3 Hz, 1H), 4.46 (d, J = 12.1 Hz, 1H), 4.14 (d, J = 14.2 Hz, 1H), 3.98 (s, 3H), 3.79-3.72 (m, 3H), 3.71-3.66 (m, 4H), 3.63- 3.57 (m, 2H), 3.27-3.201 (m, 1H), 2.86 (t, J = 11.9 Hz, 1H), 2.14-2.02 (m, 2H), 1.91-1.80 |
| | | (m, 2H), 1.51-1.37 (m, 2H). |
| 107 | 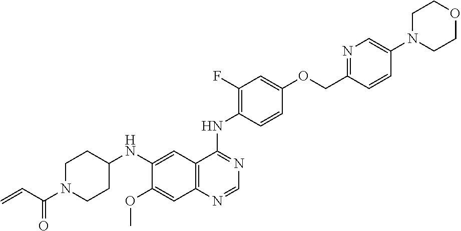 [SMILES: C=CC(=O)N1CCC(Nc2cc3c(Nc4ccc(OCc5ccc(N6CCOCC6)cn5)cc4F)ncnc3cc2OC)CC1] | LC/MS: 614.2 [M + H]⁺. 1H NMR (400 MHz, DMSO) δ 9.07 (s, 1H), 8.39- 8.26 (m, 2H), 8.15 (s, 1H), 7.45-7.30 (m, 3H), 7.25 (s, 1H), 7.10-7.00 (m, 2H), 6.92-6.81 (m, 2H), 6.11 (dd, J = 16.7, 2.5 Hz, 1H), 5.68 (dd, J = 10.4, 2.5 Hz, 1H), 5.28 (d, J = 8.5 Hz, 1H), 5.11 (s, 2H), 4.46 (d, J = 13.2 Hz, 1H), 4.13 (d, J = 12.5 Hz, 1H), 3.95 (s, 3H), 3.81-3.68 (m, 5H), 3.28-3.15 (m, 5H), 2.88-2.80 (m, 1H), 2.10-2.00 (m, 2H), 1.50-1.35 (m, 2H). |
| 108 | 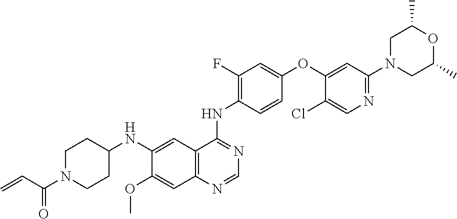 [SMILES: C=CC(=O)N1CCC(Nc2cc3c(Nc4ccc(Oc5cc(N6C[C@@H](C)O[C@@H](C)C6)ncc5Cl)cc4F)ncnc3cc2OC)CC1] | LC/MS: 662.2 [M + H]⁺. 1H NMR (400 MHz, DMSO) δ 8.32 (s, 1H), 8.24 (s, 1H), 7.56 (t, J = 8.8 Hz, 1H), 7.40 (s, 1H), 7.19 (dd, J = 11.0, 2.7 Hz, 1H), 7.11 (s, 1H), 6.97 (dd, J = 8.6, 2.3 Hz, 1H), 6.86 (dd, J = 16.7, 10.5 Hz, 1H), 6.55 (s, 1H), 6.11 (dd, J = 16.7, 2.5 Hz, 1H), 5.68 (dd, J = 10.4, 2.5 Hz, 1H), 5.55-5.45 (m, 1H), 4.46 (d, J = 11.4 Hz, 1H), 4.13 (d, J = 15.3 Hz, 1H), 4.07-3.94 (m, 5H), 3.86-3.76 (m, 1H), 3.61-3.54 (m, 2H), 3.30- 3.15 (m, 2H), 2.90-2.80 (m, 1H), 2.43-2.36 (m, 2H), 2.10-2.00 (m, 2H), 1.50-1.40 (m, 2H), 1.14 (d, J = 11.6 Hz, 6H). |
| 109 | 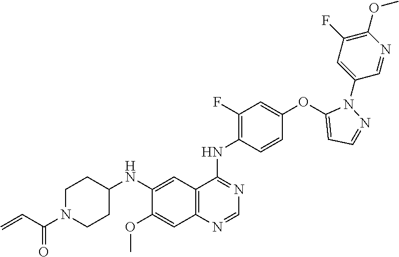 [SMILES: C=CC(=O)N1CCC(Nc2cc3c(Nc4ccc(Oc5ccnn5-c5cnc(OC)c(F)c5)cc4F)ncnc3cc2OC)CC1] | LC/MS: 629.2 [M + H]⁺. ¹H NMR (400 MHz, DMSO) δ 9.04 (s, 1H), 8.32 (d, J = 2.3 Hz, 1H), 8.30 (d, J = 2.3 Hz, 1H), 8.26 (brs, 1H), 8.16 (s, 1H), 7.98 (dd, J = 11.3, 2.3 Hz, 1H), 7.59 (dd, J = 8.8, 6.3 Hz, 1H), 7.20- 7.09 (m, 3H), 7.01 (s, 1H), 6.83 (dd, J = 16.7, 10.5 Hz, 1H), 6.14-6.07 (m, 2H), 5.68 (dd, J = 10.4, 2.4 Hz, 1H), 5.22 (d, J = 8.6 Hz, 1H), 4.42 (d, J = 13.2 Hz, 1H), 4.07 (d, J = 13.7 Hz, 1H), 3.95 (s, 3H), 3.93 (s, 3H), 3.68-3.64 (m, 1H), 3.20-3.12 (m, 1H), 2.80 (t, J = 11.9 Hz, 1H), 2.05-1.95 (m, 2H), 1.43-1.32 (m, 2H). |
| 110 | 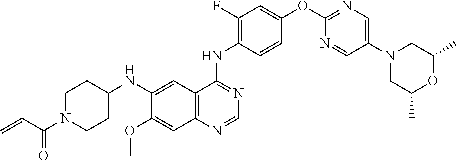 [SMILES: C=CC(=O)N1CCC(Nc2cc3c(Nc4ccc(Oc5ncc(N6C[C@@H](C)O[C@@H](C)C6)cn5)cc4F)ncnc3cc2OC)CC1] | LC/MS: 629.3 [M + H]⁺. 1H NMR (400 MHz, DMSO) δ 9.17 (s, 1H), 8.40 (s, 2H), 7.51 (t, J = 8.8 Hz, 1H), 7.26-7.20 (m, 2H), 7.08 (s, 1H), 7.04 (dd, J = 8.6, 1.7 Hz, 1H), 6.86 (dd, J = 16.7, 10.5 Hz, 1H), 6.12 (dd, J = 16.7, 2.5 Hz, 1H), 5.69 (dd, J = 10.4, 2.5 Hz, 1H), 5.33 (d, J = 8.6 Hz, 1H), 4.46 (d, J = 12.6 Hz, 1H), 4.13 (d, J = 12.6 Hz, 1H), 3.96 (s, 3H), 3.74-3.71 (m, 2H), 3.61 (d, J = 10.5 Hz, 2H), 3.28-3.13 (m, 2H), 2.86 (t, J = 12.3 Hz, 1H), |
| | | 2.33 (d, J = 10.7 Hz, 2H), 2.12-2.05 (m, 2H), |
| | | 1.49-1.41 (m, 2H), 1.15 (d, J = 6.2 Hz, 6H). |
| 111 | 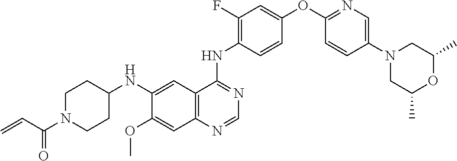 [SMILES: C=CC(=O)N1CCC(Nc2cc3c(Nc4ccc(Oc5ccc(N6C[C@@H](C)O[C@@H](C)C6)cn5)cc4F)ncnc3cc2OC)CC1] | LC/MS: 628.3 [M + H]⁺. ¹H NMR (400 MHz, DMSO) δ 10.75 (s, 1H), 8.70 (s, 1H), 7.90 (d, J = 3.0 Hz, 1H), 7.59 (dd, J = 9.0, 3.1 Hz, 1H), 7.54 (t, J = 8.8 Hz, 1H), 7.43 (s, 1H), 7.22-7.15 (m, 2H), 7.08-6.99 (m, 2H), 6.87 (dd, J = 16.7, 10.5 Hz, 1H), 6.13 (dd, J = 16.7, 2.4 Hz, 1H), 6.03 (d, J = 8.0 Hz, 1H), 5.70 (dd, J = 10.4, 2.4 Hz, 1H), 4.49 (d, J = 12.4 Hz, 1H), 4.16 (d, J = 14.1 Hz, 1H), 4.04 (s, 3H), 3.73-3.69 (m, 3H), 3.58 (d, J = 10.6 Hz, 2H), 3.24-3.17 (m, 1H), 2.83 (t, J = 12.0 Hz, 1H), |
| | | 2.31-2.26 (m, 2H), 2.06-2.02 (m, 2H), 1.55- |
| | | 1.42 (m, 2H), 1.16 (d, J = 6.2 Hz, 6H). |
| 112 | 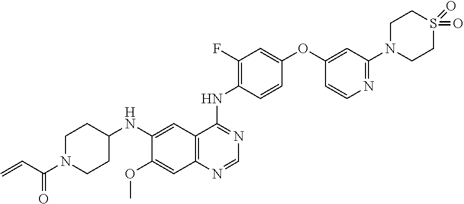 [SMILES: C=CC(=O)N1CCC(Nc2cc3c(Nc4ccc(Oc5ccnc(N6CCS(=O)(=O)CC6)c5)cc4F)ncnc3cc2OC)CC1] | LC/MS: 648.1 [M + H]⁺. 1H NMR (400 MHz, DMSO) δ 9.18 (s, 1H), 8.22 (s, 1H), 8.12 (d, J = 5.7 Hz, 1H), 7.58 (t, J = 8.8 Hz, 1H), 7.29-7.19 (m, 2H), 7.11-7.03 (m, 2H), 6.86 (dd, J = 16.7, 10.5 Hz, 1H), 6.72 (d, J = 1.9 Hz, 1H), 6.34 (dd, J = 5.7, 2.0 Hz, 1H), 6.12 (dd, J = 16.7, 2.5 Hz, 1H), 5.69 (dd, J = 10.4, 2.5 Hz, 1H), 5.34 (d, J = 8.5 Hz, 1H), 4.51- 4.40 (m, 1H), 4.19-4.09 (m, 1H), 4.08-4.02 (m, 4H), 3.98-3.94 (m, 3H), 3.80-3.70 (m, 1H), 3.22 (t, J = 12.6 Hz, 1H), 3.14-3.10 (m, 4H), 2.86 (t, J = 11.3 Hz, 1H), 2.08-2.04 (m, 2H), 1.50-1.38 (m, 2H). |
| 113 | 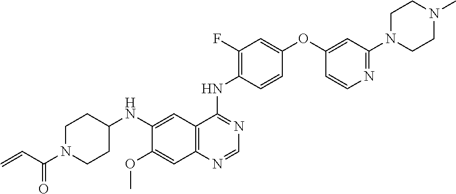 [SMILES: C=CC(=O)N1CCC(Nc2cc3c(Nc4ccc(Oc5ccnc(N6CCN(C)CC6)c5)cc4F)ncnc3cc2OC)CC1] | LC/MS: 613.1 [M + H]⁺. 1H NMR (400 MHz, DMSO) δ 10.83 (s, 1H), 9.96 (brs, 1H), 8.70 (s, 1H), 8.15 (d, J = 5.7 Hz, 1H), 7.63 (t, J = 8.7 Hz, 1H), 7.47 (s, 1H), 7.32 (dd, J = 10.9, 2.7 Hz, 1H), 7.21 (s, 1H), 7.11 (dd, J = 8.6, 1.8 Hz, 1H), 6.87 (dd, J = 16.7, 10.5 Hz, 1H), 6.70 (d, J = 1.9 Hz, 1H), 6.37 (dd, J = 5.7, 2.0 Hz, 1H), 6.13 (dd, J = 16.7, 2.4 Hz, 1H), 6.10-6.02 (m, 1H), 5.70 (dd, J = 10.4, 2.4 Hz, 1H), 4.51-4.32 (m, 4H), 4.16 (d, J = 12.1 Hz, 2H), 4.04 (s, 3H), 3.51 (d, J = 11.7 Hz, 2H), 3.25-3.06 (m, 5H), 2.85 (s, 3H), 2.04 (d, J = 10.7 Hz, 2H), 1.57-1.47 (m, 2H). |
| 114 | 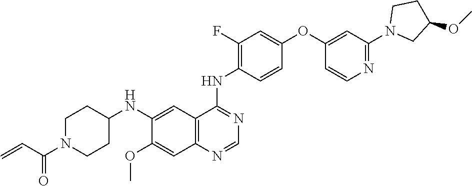 [SMILES: C=CC(=O)N1CCC(Nc2cc3c(Nc4ccc(Oc5ccnc(N6CC[C@@H](OC)C6)c5)cc4F)ncnc3cc2OC)CC1] | LC/MS: 614.1 [M + H]⁺. ¹H NMR (400 MHz, DMSO) δ 10.85 (s, 1H), 8.69 (s, 1H), 8.06 (d, J = 6.5 Hz, 1H), 7.68 (t, J = 8.7 Hz, 1H), 7.50 (s, 1H), 7.44 (d, J = 8.5 Hz, 1H), 7.21 (d, J = 8.5 Hz, 2H), 6.87 (dd, J = 16.7, 10.5 Hz, 1H), 6.44 (s, 1H), 6.31 (s, 1H), 6.12 (dd, J = 16.7, 2.4 Hz, 1H), 6.01 (d, J = 8 Hz, 1H), 5.70 (dd, J = 10.4, 2.4 Hz, 1H), 4.48 (d, J = 12.0 Hz, 1H), 4.17-4.11 (m, 2H), 4.04 (s, 3H), 3.80-3.78 (m, 1H), 3.45-3.43 (m, 2H), 3.41-3.38 (m, 2H), 3.35-3.32 (s, 1H), 3.27 (s, 3H), 2.87-2.80 (m, 1H), 2.15-2.00 (m, 4H), |
| | | 1.54-1.34 (m, 2H). |
| 115 | 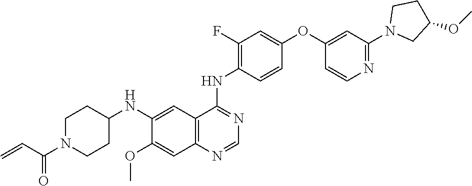 [SMILES: C=CC(=O)N1CCC(Nc2cc3c(Nc4ccc(Oc5ccnc(N6CC[C@H](OC)C6)c5)cc4F)ncnc3cc2OC)CC1] | LC/MS: 614.1 [M + H]⁺. ¹H NMR (400 MHz, DMSO) δ 8.44 (brs, 1H), 8.03 (d, J = 5.8 Hz, 1H), 7.59 (t, J = 8.8 Hz, 1H), 7.41 (brs, 1H), 7.26 (dd, J = 11.0, 2.7 Hz, 1H), 7.12 (s, 1H), 7.07 (dd, J = 8.7, 1.8 Hz, 1H), 6.87 (dd, J = 16.7, 10.5 Hz, 1H), 6.24 (dd, J = 5.8, 2.1 Hz, 1H), 6.15-6.08 (m, 2H), 5.73-5.68 (m, 1H), 5.63 (d, J = 7.9 Hz, 1H), 4.47 (d, J = 12.6 Hz, 1H), 4.14 (d, J = 13.2 Hz, 1H), 4.08-4.03 (m, 1H), 4.00 (s, 3H),3.84-3.75 (m, 1H), 3.46 (d, J = 2.8 Hz, 3H), 3.42-3.37 (m, 2H), 3.29- 3.27 (m, 2H), 3.24-3.26 (m, 2H), 2.85 (t, J = |
| | | 13.2 Hz, 1H), 2.09-2.01 (m, 4H), 1.49-1.41 |
| | | (m, 2H). |
| 116 | 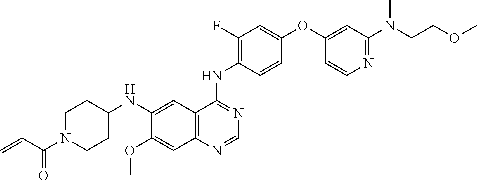 [SMILES: C=CC(=O)N1CCC(Nc2cc3c(Nc4ccc(Oc5ccnc(N(C)CCOC)c5)cc4F)ncnc3cc2OC)CC1] | LC/MS: 602.0 [M + H]⁺. ¹H NMR (400 MHz, DMSO) δ 8.32-8.23 (m, 1H), 8.02 (d, J = 6.2 Hz, 1H), 7.57 (t, J = 8.8 Hz, 1H), 7.29 (s, 1H), 7.22 (dd, J = 11.6 Hz, 1H), 7.10-7.02 (m, 3H), 6.92-6.81 (m, 1H),6.25- 6.05 (m, 4H), 5.75-5.63 (m, 1H), 4.50-4.42 (m, 1H),4.16-4.11 (m, 1H), 3.97 (s, 3H), 3.68 (t, J = 5.7 Hz, 2H), 3.48 (t, J = 5.7 Hz, 3H), 3.24 (s, 3H), 2.98 (s, 3H), 2.10-2.02 (m, 2H), 1.51- 1.41 (m, 4H). |
| 117 | 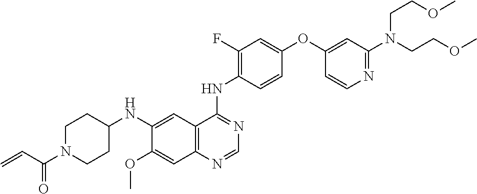 [SMILES: C=CC(=O)N1CCC(Nc2cc3c(Nc4ccc(Oc5ccnc(N(CCOC)CCOC)c5)cc4F)ncnc3cc2OC)CC1] | LC/MS: 646.3 [M + H]⁺. 1H NMR (400 MHz, DMSO) δ 8.29 (s, 1H), 8.01 (d, J = 5.7 Hz, 1H), 7.57 (t, J = 8.7 Hz, 1H), 7.31 (s, 1H), 7.22 (dd, J = 11.0, 2.7 Hz, 1H), 7.11- 7.01 (m, 2H), 6.86 (dd, J = 16.6, 10.5 Hz, 1H), 6.28 (d, J = 1.9 Hz, 1H), 6.21 (dd, J = 5.7, 2.0 Hz, 1H), 6.12 (dd, J = 16.7, 2.5 Hz, 1H), 5.69 (dd, J = 10.4, 2.5 Hz, 1H), 5.46-5.41 (m, 1H), 4.47-4.43 (m, 1H), 4.14 (d, J = 11.7 Hz, 1H), 3.97 (s, 3H), 3.79-3.72 (m, 1H), 3.67-3.58 (m, 4H), 3.51-3.45 (m, 4H), 3.32-3.31 (m, 2H), 3.24 (s, 6H), 2.90-2.83 (m, 1H), 2.10-2.02 (m, 2H), 1.50-1.41 (m, 2H). |
| 118 | 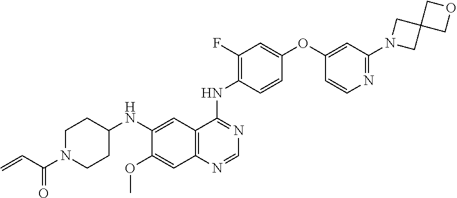 [SMILES: C=CC(=O)N1CCC(Nc2cc3c(Nc4ccc(Oc5ccnc(N6CC7(COC7)C6)c5)cc4F)ncnc3cc2OC)CC1] | LC/MS: 612.3 [M + H]⁺. 1H NMR (400 MHz, DMSO) δ 8.57 (s, 1H), 8.04 (d, J = 5.9 Hz, 1H), 7.62 (t, J = 8.7 Hz, 1H), 7.48 (s, 1H), 7.34-7.28 (m, 1H), 7.15 (s, 1H), 7.10 (d, J = 8.5 Hz, 1H), 6.87 (dd, J = 16.7, 10.5 Hz, 1H), 6.34 (d, J = 5.1 Hz, 1H), 6.12 (dd, J = 16.6, 2.4 Hz, 1H), 5.98-5.95 (m, 1H), 5.89-5.81 (m, 1H), 5.70 (dd, J = 10.4, 2.4 Hz, 1H), 5.35-5.29 (m, 1H), 4.73-4.69 (m, 2H), 4.53-4.48 (m, 1H), 4.15-4.10 (m, 1H), 4.10 (s, 3H), 4.02 (s, 2H), 3.81-3.76 (m, 1H), 2.88-2.82 (m, 2H), 2.10-1.91 (m, 4H), 1.55-1.47 (m, 2H), 1.32- 1.25 (m, 2H). |
| 119 | 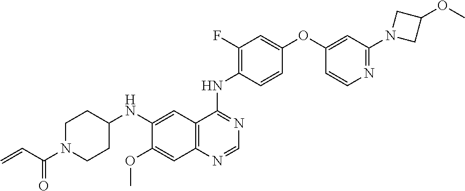 [SMILES: C=CC(=O)N1CCC(Nc2cc3c(Nc4ccc(Oc5ccnc(N6CC(OC)C6)c5)cc4F)ncnc3cc2OC)CC1] | LC/MS: 600.3 [M + H]⁺. 1H NMR (400 MHz, DMSO) δ 8.32 (s, 1H), 8.03 (d, J = 5.8 Hz, 1H), 7.59 (t, J = 8.7 Hz, 1H), 7.30 (s, 1H), 7.25 (d, J = 10.8 Hz, 1H), 7.10-7.04 (m, 2H), 6.87 (dd, J = 16.6, 10.5 Hz, 2H), 6.30 (dd, J = 5.8, 2.2 Hz, 1H), 6.12 (dd, J = 16.7, 2.4 Hz, 2H), 5.95 (d, J = 2.1 Hz, 1H), 5.69 (dd, J = 10.4, 2.5 Hz, 1H), 4.52-4.42 (m, 2H), 4.32- 4.28 (m, 1H), 4.13-4.08 (m, 2H), 3.98 (s, 3H), 3.72 (dd, J = 9.3, 4.3 Hz, 3H), 3.24 (s, 3H), 2.89- 2.80 (m, 2H), 2.13-2.05 (m, 2H), 1.49-1.42 (m, 2H). |
| 120 | 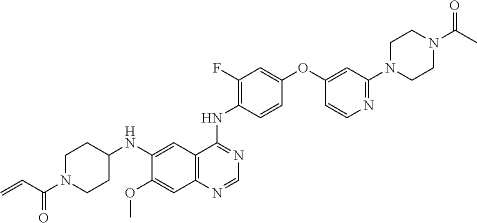 [SMILES: C=CC(=O)N1CCC(Nc2cc3c(Nc4ccc(Oc5ccnc(N6CCN(C(C)=O)CC6)c5)cc4F)ncnc3cc2OC)CC1] | LC/MS: 641.3 [M + H]⁺. ¹H NMR (400 MHz, DMSO) δ 9.20 (s, 1H), 8.27 (s, 1H), 8.22 (s, 1H), 8.09 (d, J = 5.6 Hz, 1H), 7.58 (t, J = 8.7 Hz, 1H), 7.32-7.15 (m, 2H), 7.11-6.99 (m, 2H), 6.87 (dd, J = 16.8, 10.4 Hz, 1H), 6.47 (s, 1H), 6.28 (d, J = 4.3 Hz, 1H), 6.11 (d, J = 16.7 Hz, 1H), 5.69 (d, J = 12.6 Hz, 1H), 5.34 (d, J = 8.4 Hz, 1H), 4.46 (d, J = 11.7 Hz, 1H), 4.14 (d, J = 13.5 Hz, 1H), 3.96 (s, 3H), 3.78-3.72 (m, 1H), 3.55-3.48 (m, 9H), 2.86 (t, J = 12.4 Hz, 1H), 2.12-2.02 (m, 5H), 1.45-1.42 (m, 2H). |
| 121 | 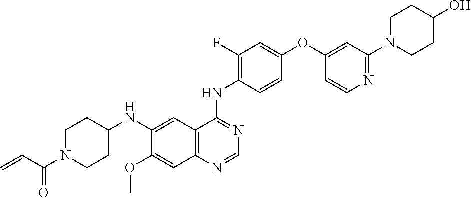 | LC/MS: 614.3 [M + H]⁺. ¹H NMR (400 MHz, DMSO) δ 9.20 (s, 1H), 8.21 (s, 1H), 8.04 (d, J = 5.7 Hz, 1H), 7.57 (t, J = 8.6 Hz, 1H), 7.33-7.16 (m, 2H), 7.11-6.99 (m, 2H), 6.86 (dd, J = 16.6, 10.4 Hz, 1H), 6.44 (d, J = 12.6 Hz, 1H), 6.29-6.09 (m, 2H), 5.69 (d, J = 10.4 Hz, 1H), 5.35 (d, J = 8.5 Hz, 1H), 4.73 (d, J = 4.2 Hz, 1H), 4.46 (d, J = 13.7 Hz, 1H), 4.14 (d, J = 12.7 Hz, 1H), 4.09-3.86 (m, 4H), 3.82-3.62 (m, 2H), 3.30-3.17 (m, 1H), 3.06 (t, J = 10.8 Hz, 2H), 2.95-2.78 (m, 1H), 2.15- 1.97 (m, 2H), 1.85-1.68 (m, 2H), 1.56-1.23 (m, 4H). |
| 122 | 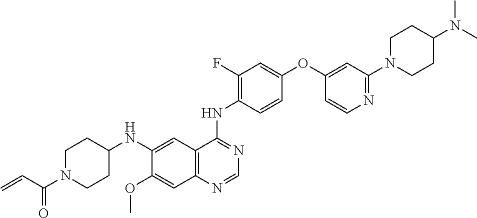 | LC/MS: 641.1 [M + H]⁺. ¹H NMR (400 MHz, DMSO) δ 8.69 (s, 1H), 8.10 (d, J = 5.7 Hz, 1H), 7.63 (t, J = 8.9 Hz, 1H), 7.45 (s, 1H), 7.32 (dd, J = 8.3 Hz, 1H), 7.16 (s, 1H), 7.13-7.09(m, 1H), 6.87 (dd, J = 16.7, 10.5 Hz, 2H), 6.66-6.63 (m, 1H), 6.27 (dd, J = 4.0 Hz, 1H), 6.13 (dd, J = 16.6, 2.5 Hz, 1H), 5.70 (dd, J = 10.6, 2.4 Hz, 1H), 4.45 (d, J = 12.6 Hz, 3H), 4.04 (s, 2H), 2.90-2.82 (m, 3H), 2.77 (d, J = 5.0 Hz, 4H), 2.49-2.46 (m, 6H), 2.04 (d, J = 11.1 Hz, 4H), 1.59-1.46 (m, 4H), 1.25-1.22 (m, 1H), 1.16-1.13 (m, 1H). |
| 123 | 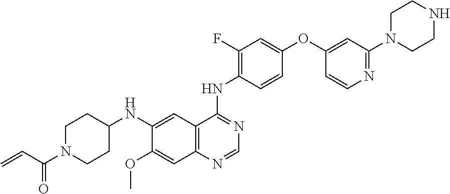 | LC/MS: 599.1 [M + H]⁺. ¹H NMR (400 MHz, DMSO) δ 9.19 (s, 1H), 8.22 (s, 3H), 8.07 (d, J = 5.7 Hz, 1H), 7.58 (t, J = 8.8 Hz, 1H), 7.26 (s, 1H), 7.21 (dd, J = 10.9, 2.7 Hz, 1H), 7.08 (s, 1H), 7.03 (dd, J = 8.6, 1.8 Hz, 1H), 6.87 (dd, J = 16.7, 10.5 Hz, 1H), 6.43 (d, J = 1.9 Hz, 1H), 6.27 (dd, J = 5.7, 2.0 Hz, 1H), 6.12 (dd, J = 16.7, 2.5 Hz, 1H), 5.69 (dd, J = 10.4, 2.4 Hz, 1H), 5.34 (d, J = 8.5 Hz, 1H), 4.46 (d, J = 12.5 Hz, 1H), 4.14 (d, J = 12.3 Hz, 1H), 3.96 (s, 3H), 3.80-3.71 (m, 1H), 3.48-3.44 (m, 4H), 3.27- 3.18 (m, 2H), 2.88-2.84 (m, 4H), 2.10-2.03 |
| | | (m, 2H), 1.50-1.37 (m, 2H). |
| 124 | 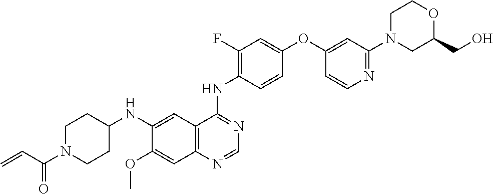 | LC/MS: 630.0 [M + H]⁺. ¹H NMR (400 MHz,) δ 9.82 (br, 1H), 8.36 (s, 1H), 8.10 (d, J = 5.7 Hz, 1H), 7.60 (t, J = 8.8 Hz, 1H), 7.40 (s, 1H), 7.24 (dd, J = 10.9, 2.7 Hz, 1H), 7.15-7.11 (m, 1H), 7.09-7.04 (m, 1H), 6.87 (dd, J = 16.7, 10.5 Hz, 1H), 6.44 (d, J = 2.0 Hz, 1H), 6.31 (dd, J = 5.7, 2.0 Hz, 1H), 6.12 (dd, J = 16.6, 2.5 Hz, 1H), 5.69 (dd, J = 10.4, 2.5 Hz, 1H), 5.52 (d, J = 7.7 Hz, 1H), 4.80 (t, J = 4.9 Hz, 1H), 4.47 (d, J = 11.7 Hz, 1H), 4.16 (t, J = 14.8 Hz, 2H), 3.98 (s, 3H), 3.96-3.91 (m, 1H), 3.85- 3.75 (m, 1H), 3.59-3.36 (m, 5H), 3.26 (t, J = 12.9 Hz, 1H), 2.92-2.78 (m, 2H), 2.59-2.53 |
| | | (m, 1H), 2.09-2.03 (m, 2H), 1.49-1.41 (m, |
| | | 2H). |
| 125 | 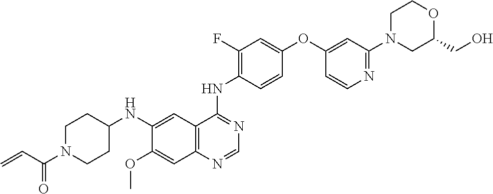 | LC/MS: 630.0 [M + H]⁺. NMR (400 MHz,) δ 9.02 (brs, 1H), 8.27 (s, 1H), 8.06 (d, J = 5.7 Hz, 1H), 7.57-7.52 (m, 1H), 7.33-7.27 (m, 1H), 7.20 (dd, J = 10.8, 2.5 Hz, 1H), 7.06 (s, 1H), 7.01-6.92 (m, 1H), 6.86- 6.79 (m, 1H), 6.39 (d, J = 1.6 Hz, 1H), 6.27 (dd, J = 5.7, 1.8 Hz, 1H), 6.08 (dd, J = 16.7, 2.4 Hz, 1H), 5.65 (dd, J = 10.4, 2.4 Hz, 1H), 5.48-5.26 (m, 1H), 4.79-4.75 (m, 1H), 4.43 (d, J = 10.9 Hz, 1H), 4.16-4.08 (m, 2H), 3.94 (s, 3H), 3.90- 3.86 (m, 1H), 3.77-3.71 (m, 1H), 3.51-3.39 (m, 5H), 3.24-3.21 (m, 1H), 2.79 (dd, J = 11.8, 9.2 Hz, 2H), 2.66-2.62 (m, 1H), 2.04-1.99 (m, |
| | | 2H), 1.43-1.39 (m, 2H). |
| 126 | 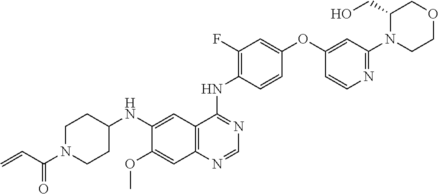 | LC/MS: 630.0 [M + H]⁺. 1H NMR (400 MHz, DMSO) δ 9.18 (s, 1H), 8.22- 8.20 (m, 2H), 8.06 (d, J = 5.7 Hz, 1H), 7.57 (t, J = 8.8 Hz, 1H), 7.33-7.18 (m, 2H), 7.08-6.99 (m, 2H), 6.86 (dd, J = 16.7, 10.5 Hz, 1H), 6.38 (d, J = 1.9 Hz, 1H), 6.24 (dd, J = 5.7, 2.0 Hz, 1H), 6.12 (dd, J = 16.7, 2.5 Hz, 1H), 5.69 (dd, J = 10.4, 2.5 Hz, 1H), 5.34 (d, J = 8.5 Hz, 1H), 4.46 (d, J = 11.9 Hz, 1H), 4.21-4.02 (m, 3H), 3.96 (s, 3H), 3.94-3.78 (m, 2H), 3.76-3.72 (m, 1H), 3.68 (t, J = 10.0 Hz, 1H), 3.48-3.43 (m, 2H), 3.37-3.33 (m, 1H), 3.27-3.17 (m,1H), 3.05-2.93 (m, 1H), 2.85 (t, J = 11.6 Hz, 1H), |
| | | 2.14-2.02 (m, 2H), 1.52-1.35 (m, 2H). |
| 127 | 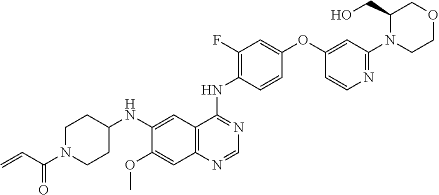 | LC/MS: 630.0 [M + H]⁺. ¹H NMR (400 MHz, DMSO) δ 9.18 (s, 1H), 8.22- 8.20 (m, 2H), 8.06 (d, J = 5.7 Hz, 1H), 7.57 (t, J = 8.8 Hz, 1H), 7.33-7.18 (m, 2H), 7.08-6.99 (m, 2H), 6.86 (dd, J = 16.7, 10.5 Hz, 1H), 6.38 (d, J = 1.9 Hz, 1H), 6.24 (dd, J = 5.7, 2.0 Hz, 1H), 6.12 (dd, J = 16.7, 2.5 Hz, 1H), 5.69 (dd, J = 10.4, 2.5 Hz, 1H), 5.34 (d, J = 8.5 Hz, 1H), 4.46 (d, J = 11.9 Hz, 1H), 4.21-4.15 (m, 1H), 4.14-4.03 (m, 2H),3.96 (s, 3H), 3.94-3.78 (m, 2H), 3.76-3.72 (m, 1H), 3.68 (t, J = 10.0 Hz, 1H), 3.48-3.43 (m, 2H), 3.37-3.33 (m, 1H), 3.27-3.17 (m,1H), 3.05-2.93 (m, 1H), 2.85 (t, |
| | | J = 11.6 Hz, 1H), 2.14-2.02 (m, 2H), 1.52- |
| | | 1.35 (m, 2H). |
| 128 | 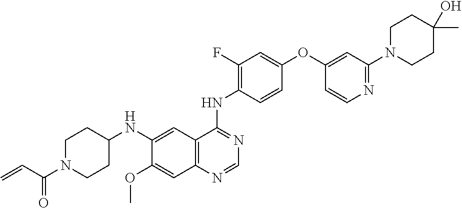 | LC/MS: 628.2 [M + H]⁺. ¹H NMR (400 MHz, DMSO) δ 9.24 (s, 1H), 8.24 (s, 1H), 8.14 (s, 1H), 8.04 (d, J = 5.6 HZ 1H), 7.57 (t, J = 8.8 Hz, 1H), 7.26 (s, 1H), 7.21 (dd, J = 10.8, 2.4 Hz, 1H), 7.08 (s, 1H), 7.05-6.99 (m, 1H), 6.86 (dd, J = 16.8, 10.4 Hz, 1H), 6.41 (d, J = 2.0 Hz, 1H), 6.19 (dd, J = 5.6, 2.0 Hz, 1H), 6.12 (dd, J = 16.8, 2.4 Hz, 1H), 5.69 (dd, J = 10.4, 2.4 Hz, 1H), 5.37 (d, J = 8.0 Hz, 1H), 4.46 (d, J = 11.2 Hz, 1H), 4.14 (d, J = 11.2 Hz, 1H), 3.95 (s, 3H), 3.83-3.70 (m, 3H), 3.31- 3.18 (m, 4H), 2.85 (t, J = 12.0 Hz, 1H), 2.17 2.03 (m, 2H), 1.54-1.38 (m, 6H), 1.14 (s, 3H). |
| 129 | 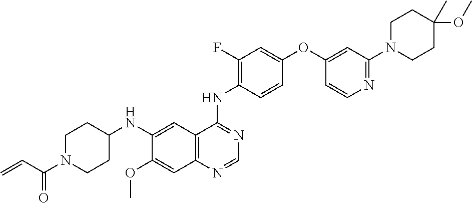 | LC/MS: 642.3 [M + H]⁺. ¹H NMR (400 MHz, DMSO) δ 9.19 (s, 1H), 8.22 (s, 1H), 8.05 (d, J = 5.8 Hz, 1H), 7.57 (t, J = 8.8 Hz, 1H), 7.26 (s, 1H), 7.22-7.18 (m, 1H), 7.08 (s, 1H), 7.03-7.00 (m, 1H), 6.90-6.82 (m, 1H), 6.42 (d, J = 2.0 Hz, 1H), 6.21 (dd, J = 5.6, 2.0 Hz, 1H), 6.14-6.09 (m, 1H), 5.69 (dd, J = 10.4, 2.4 Hz, 1H), 5.34 (d, J = 8.4 Hz, 1H), 4.46 (d, J = 10.8 Hz, 1H), 4.14 (d, J = 13.2 Hz, 1H), 3.96 (s, 3H), 3.81-3.76 (m, 2H), 3.24-3.14 (m, 4H), 3.13 (s, 3H), 2.84 (d, J = 11.2 Hz, 1H), 2.13- 2.02 (m, 2H), 1.70 (d, J = 13.5 Hz, 2H), 1.51- 1.38 (m, 4H), 1.12 (s, 3H). |
| 130 | 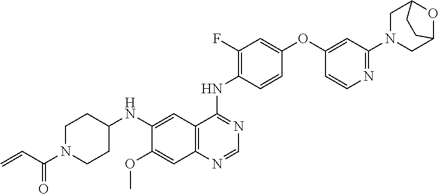 | LC/MS: 626.2 [M + H]⁺. ¹H NMR (400 MHz, DMSO) δ 9.53 (brs, 1H), 8.32 (s, 1H), 8.06 (d, J = 5.7 Hz, 1H), 7.58 (t, J = 8.8 Hz, 1H), 7.31 (s, 1H), 7.24 (dd, J = 11.0, 2.6 Hz, 1H), 7.09 (s, 1H), 7.05 (dd, J = 8.7, 1.9 Hz, 1H), 6.87 (dd, J = 16.7, 10.5 Hz, 1H), 6.32 (d, J = 1.9 Hz, 1H), 6.28 (dd, J = 5.6, 2.0 Hz, 1H), 6.12 (dd, J = 16.7, 2.5 Hz, 1H), 5.69 (dd, J = 10.4, 2.5 Hz, 1H), 5.49 (d, J = 7.5 Hz, 1H), 4.52-4.39 (m, 3H), 4.14 (d, J = 14.4 Hz, 1H), 3.98 (s, 3H), 3.77 (d, J = 11.9 Hz, 3H), 3.23 (t, J = 12.6 Hz, 1H), 2.92 (dd, J = 12.4, 2.4 Hz, 2H), 2.85 (t, J = 11.9 Hz, 1H), 2.11-2.02 (m, |
| | | 2H), 1.86-1.77 (m, 2H), 1.77-1.68 (m, 2H), |
| | | 1.52-1.43 (m, 2H). |
| 131 | 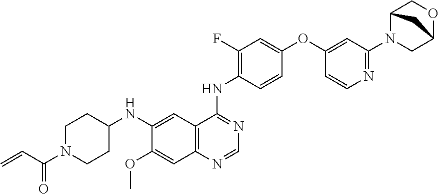 | LC/MS: 612.2 [M + H]⁺. ¹H NMR (400 MHz, DMSO) δ 10.82 (s, 1H), 8.71 (s, 1H), 8.06 (d, J = 6.8 Hz, 1H), 7.69 (t, J = 8.8 Hz, 1H), 7.50-7.40 (m, 2H), 7.22 (d, J = 12.8 Hz, 2H), 6.87 (dd, J = 16.8, 10.4 Hz, 1H), 6.44 (d, J = 5.6 Hz, 1H), 6.16-6.02 (m, 2H), 5.70 (dd, J = 10.4, 2.4 Hz, 1H), 4.96 (s, 1H), 4.74 (s, 1H), 4.48 (d, J = 11.6 Hz, 1H), 4.16 (d, J = 12.8 Hz, 1H), 4.05 (s, 3H), 3.81-3.73 (m, 3H), 3.52 (d, J = 10.0 Hz, 1H), 3.32-3.18 (m, 2H), 2.84 (t, J = 11.6 Hz, 1H), 2.08-2.00 (m, 2H), 1.95 (s, 2H), 1.63-1.52 (m, 2H). |
| 132 | 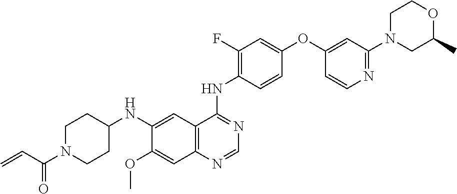 | LC/MS: 614.3 [M + H]⁺. ¹H NMR (400 MHz, DMSO) δ 9.17 (s, 1H), 8.22 (s, 1H), 8.08 (d, J = 5.7 Hz, 1H), 7.57 (t, J = 8.8 Hz, 1H), 7.28-7.16 (m, 2H), 7.09-6.99 (m, 2H), 6.86 (dd, J = 16.7, 10.5 Hz, 1H), 6.46 (d, J = 1.8 Hz, 1H), 6.28 (dd, J = 5.6, 1.9 Hz, 1H), 6.12 (dd, J = 16.7, 2.4 Hz, 1H), 5.69 (dd, J = 10.4, 2.4 Hz, 1H), 5.33 (d, J = 8.5 Hz, 1H), 4.46 (d, J = 13.3 Hz, 1H), 4.12 (d, J = 12.4 Hz, 2H), 4.01-3.94 (m, 4H), 3.89 (dd, J = 11.5, 2.5 Hz, 1H), 3.81-3.72 (m, 1H), 3.59-3.48 (m, 2H), 3.22 (t, J = 12.7 Hz, 1H), 2.90-2.75 (m, 2H), 2.48-2.42 (m, 1H), 2.08-2.04 (m, 2H), 1.47- |
| | | 1.40 (m, 2H), 1.15 (d, J = 6.2 Hz, 3H). |
| 133 | 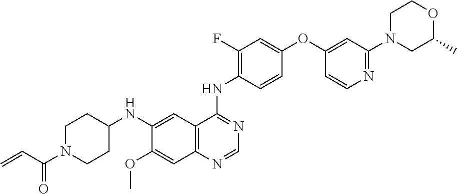 | LC/MS: 613.8 [M + H]⁺. 1H NMR (400 MHz, DMSO) δ 9.97 (brs, 1H), 8.42 (s, 1H), 8.09 (d, J = 5.7 Hz, 1H), 7.59 (t, J = 8.8 Hz, 1H), 7.41 (s, 1H), 7.25 (dd, J = 10.9, 2.6 Hz, 1H), 7.12 (s, 1H), 7.06 (dd, J = 8.7, 1.8 Hz, 1H), 6.86 (dd, J = 16.7, 10.5 Hz, 1H), 6.49 (d, J = 1.9 Hz, 1H), 6.29 (dd, J = 5.7, 2.0 Hz, 1H), 6.12 (dd, J = 16.7, 2.5 Hz, 1H), 5.69 (dd, J = 10.4, 2.5 Hz, 1H), 5.62 (d, J = 8.3 Hz, 1H), 4.47 (d, J = 13.0 Hz, 1H), 4.13 (d, J = 12.5 Hz, 2H), 4.05-3.94 (m, 4H), 3.89 (dd, J = 11.5, 2.4 Hz, 1H), 3.79 (d, J = 8.2 Hz, 1H), 3.57-3.49 (m, 2H), 3.23 (t, J = 12.3 Hz, 1H), 2.90-2.78 |
| | | (m, 2H), 2.49-2.42 (m, 1H), 2.10-2.02 (m, |
| | | 2H), 1.51-1.40 (m, 2H), 1.15 (d, J = 6.2 Hz, |
| | | 3H). |
| 134 | 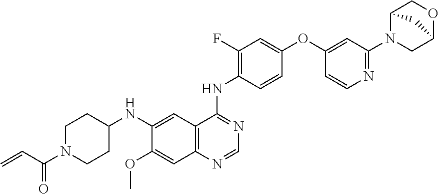 | LC/MS: 611.9 [M + H]⁺. 1H NMR (400 MHz, DMSO) δ 9.20 (s, 1H), 8.23 (s, 1H), 8.02 (d, J = 5.7 Hz, 1H), 7.57 (t, J = 8.8 Hz, 1H), 7.29-7.19 (m, 2H), 7.10-7.02 (m, 2H), 6.86 (dd, J = 16.7, 10.5 Hz, 1H), 6.22 (dd, J = 5.7, 2.1 Hz, 1H), 6.15-6.08 (m, 2H), 5.69 (dd, J = 10.4, 2.5 Hz, 1H), 5.35 (d, J = 8.5 Hz, 1H), 4.84 (s, 1H), 4.64 (s, 1H), 4.46 (d, J = 12.5 Hz, 1H), 4.13 (d, J = 12.0 Hz, 1H), 3.96 (s, 3H), 3.76 (d, J = 6.1 Hz, 2H), 3.65 (d, J = 7.3 Hz, 1H), 3.40 (d, J = 8.6 Hz, 1H), 3.25-3.14 (m, 2H), 2.86 (t, J = 13.0 Hz, 1H), 2.13-2.02 (m, 2H), 1.91-1.82 (m, 2H), 1.50-1.37 (m, 2H). |
| 135 | 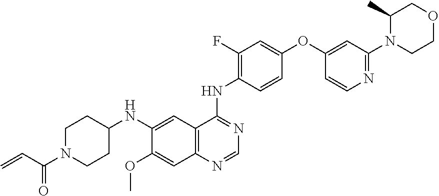 | LC/MS: 614.0 [M + H]⁺. ¹H NMR (400 MHz, DMSO) δ = 9.19 (s, 1H), 8.23 (s, 1H), 8.08 (d, J = 5.6 Hz, 1H), 7.58 (t, J = 8.8 Hz, 1H), 7.26 (s, 1H), 7.22 (dd, J = 10.8, 2.8 Hz, 1H), 7.08 (s, 1H), 7.03 (dd, J = 8.8, 2.0 Hz, 1H), 6.86 (dd, J = 16.8, 10.4 Hz, 1H), 6.36 (d, J = 2.0 Hz, 1H), 6.26 (dd, J = 5.6, 2.0 Hz, 1H), 6.12 (dd, J = 16.8, 2.4 Hz, 1H), 5.69 (dd, J = 10.4, 2.4 Hz, 1H), 5.34 (d, J = 8.8 Hz, 1H), 4.46 (d, J = 12.4 Hz, 1H), 4.32-4.25 (m, 1H), 4.13 (d, J = 10.4 Hz, 1H), 3.96 (s, 3H), 3.91 (dd, J = 11.2, 3.6 Hz, 1H), 3.81-3.68 (m, 3H), 3.60 (dd, J = 11.2, 2.8 Hz, 1H), 3.53-3.42 (m, 1H), |
| | | 3.23 (t, J = 12.4 Hz, 1H), 3.03 (td, J = 12.8, 4.0 |
| | | Hz, 1H), 2.86 (t, J = 11.6 Hz, 1H), 2.13-2.02 |
| | | (m, 2H), 1.50-1.37 (m, 2H), 1.11 (d, J = 6.4 Hz, |
| | | 3H). |
| 136 | 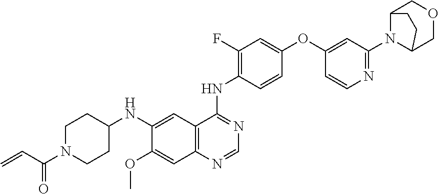 | LC/MS: 626.1 [M + H]⁺. ¹H NMR (400 MHz, DMSO) δ 9.18 (s, 1H), 8.23 (s, 1H), 8.07 (d, J = 5.6 Hz, 1H), 7.58 (t, J = 8.8 Hz, 1H), 7.26 (s, 2H), 7.23 (dd, J = 10.8, 2.8 Hz, 1H), 7.08 (s, 1H), 7.05 (d, J = 8.8 Hz, 1H), 6.87 (dd, J = 16.8, 10.4 Hz, 1H), 6.46 (d, J = 2.0 Hz, 1H), 6.24 (dd, J = 5.6, 2.0 Hz, 1H), 6.13 (dd, J = 16.8, 2.4 Hz, 1H), 5.69 (dd, J = 10.4, 2.4 Hz, 1H), 5.35 (d, J = 8.8 Hz, 1H), 4.51-4.43 (m, 3H), 4.14 (d, J = 12.8 Hz, 1H), 3.97 (s, 3H), 3.81- 3.74 (m, 1H), 3.65 (d, J = 10.4 Hz, 2H), 3.51 (d, J = 10.4 Hz, 2H), 3.24 (t, J = 12.0 Hz, 1H), 2.86 (t, J = 11.6 Hz, 1H), 2.15-2.03 (m, 2H), |
| | | 2.00-1.94 (m, 3H), 1.90-1.83 (m, 1H), 1.51- |
| | | 1.35 (m, 2H). |
| 137 | 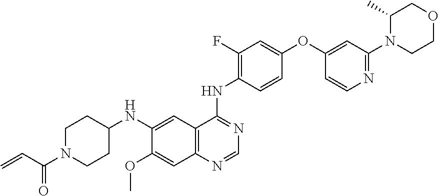 | LC/MS: 614.1 [M + H]⁺. NMR (400 MHz,) δ = 9.20 (s, 1H), 8.22 (s, 1H), 8.08 (d, J = 5.2 Hz, 1H), 7.57 (t, J = 8.8 Hz, 1H), 7.28-7.19 (m, 2H), 7.10-7.00 (m, 2H), 6.86 (dd, J = 16.8, 10.4 Hz, 1H), 6.35 (s, 1H), 6.26 (d, J = 4.4 Hz, 1H), 6.12 (d, J = 16.8 Hz, 1H), 5.69 (d, J = 10.4 Hz, 1H), 5.35 (d, J = 8.4 Hz, 1H), 4.46 (d, J = 12.8 Hz, 1H), 4.28 (d, J = 4.8 Hz, 1H), 4.13 (d, J = 11.2 Hz, 1H), 3.96 (s, 3H), 3.94-3.86 (m, 1H), 3.80-3.66 (m, 3H), 3.62- 3.54 (m, 1H), 3.52-3.44 (m, 1H), 3.24-3.16 (m, 1H), 3.03 (t, J = 11.2 Hz, 1H), 2.85 (t, J = 12.0 Hz, 1H), 2.11-2.03 (m, 2H), 1.46-1.36 |
| | | (m, 2H), 1.10 (d, J = 6.8 Hz, 3H). |
| 138 | 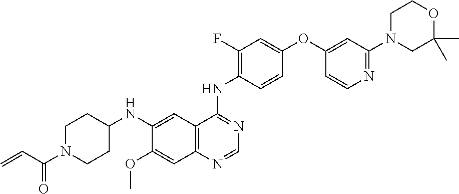 | LC/MS: 628.1 [M + H]⁺. ¹H NMR (400 MHz, DMSO) δ 9.18 (s, 1H), 8.22 (s, 1H), 8.05 (d, J = 5.7 Hz, 1H), 7.57 (t, J = 8.8 Hz, 1H), 7.26 (s, 1H), 7.21 (dd, J = 11.0, 2.6 Hz, 1H), 7.08 (s, 1H), 7.02 (dd, J = 8.7, 1.9 Hz, 1H), 6.86 (dd, J = 16.7, 10.5 Hz, 1H), 6.47 (d, J = 1.9 Hz, 1H), 6.24 (dd, J = 5.7, 2.0 Hz, 1H), 6.12 (dd, J = 16.7, 2.5 Hz, 1H), 5.69 (dd, J = 10.4, 2.5 Hz, 1H), 5.34 (d, J = 8.5 Hz, 1H), 4.46 (d, J = 13.1 Hz, 1H), 4.13 (d, J = 13.8 Hz, 1H), 3.96 (s, 3H), 3.79-3.73 (m, 1H), 3.72-3.64 (m, 2H), 3.46- 3.38 (m, 2H), 3.35 (s, 1H), 3.22 (t, J = 12.1 Hz, 1H), 2.85 (t, J = 12.3 Hz, 1H), 2.56 (s, 1H), 2.03 |
| | | (d, J = 21.2 Hz, 2H), 1.54-1.41 (m, 2H), 1.18 |
| | | (s, 6H). |
| 139 | 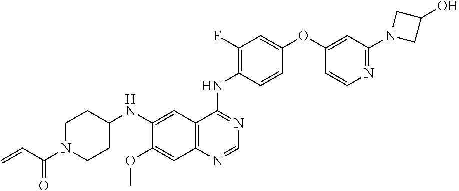 | LC/MS: 586.3 [M + H]⁺. ¹H NMR (400 MHz, DMSO) δ 10.84 (s, 1H), 8.71 (s, 1H), 8.06 (d, J = 6.7 Hz, 1H), 7.70 (t, J = 8.7 Hz, 1H), 7.51-7.43 (m, 2H), 7.27-7.20 (m, 2H), 6.87 (dd, J = 16.7, 10.5 Hz, 1H), 6.49 (d, J = 6.2 Hz, 1H), 6.19-6.02 (m, 3H), 5.70 (dd, J = 10.5, 2.4 Hz, 1H), 4.68-4.64 (m, 1H), 4.49 (d, J = 12.6 Hz, 1H), 4.31 (t, J = 7.7 Hz, 2H), 4.16 (d, J = 12.8 Hz, 1H), 4.04 (s, 3H), 3.88- 3.81 (m, 2H), 3.80-3.74 (m, 2H), 3.25-3.15 (m, 1H), 2.83 (t, J = 11.8 Hz, 1H), 2.04 (d, J = 12.3 Hz, 2H), 1.56-1.42 (m, 2H). |
| 140 | 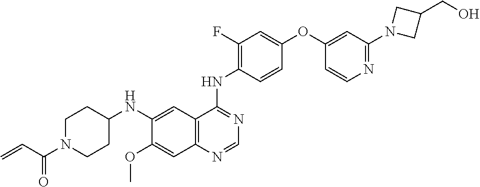 | LC/MS: 600.2 [M + H]⁺. 1H NMR (400 MHz, DMSO) δ 9.50 (brs, 1H), 8.31 (s, 1H), 8.05-7.98 (m, 1H), 7.67-7.55 (m, 1H), 7.31-7.06 (m, 3H), 6.93-6.81 (m, 1H), 6.27 (s, 1H), 6.12 (d, J = 16.6 Hz, 1H), 5.89 (s, 1H), 5.73-5.67 (m, 1H), 5.47 (s, 1H), 4.81 (s, 1H), 4.51-4.43 (m, 1H), 4.18-4.10 (m, 1H), 4.03-3.87 (m, 4H), 3.75-3.57 (m, 6H), 3.20- 3.06 (m, 2H), 2.90-2.78 (m, 2H), 2.13-2.00 (m, 2H), 1.51-1.38 (m, 2H). |
| 141 | 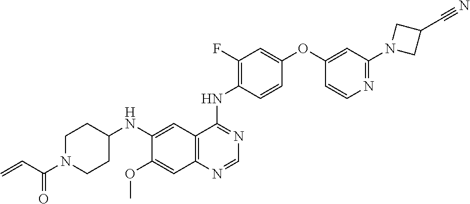 | LC/MS: 595.2 [M + H]⁺. ¹H NMR (400 MHz, DMSO) δ 9.82 (s, 1H), 8.29 (s, 1H), 8.07 (d, J = 5.8 Hz, 1H), 7.58 (t, J = 8.7 Hz, 1H), 7.51 (s, 1H), 7.22 (dd, J = 10.9, 2.5 Hz, 1H), 7.12 (s, 1H), 7.04 (dd, J = 8.6, 2.0 Hz, 1H), 6.86 (dd, J = 16.7, 10.5 Hz, 1H), 6.37 (dd, J = 5.7, 2.1 Hz, 1H), 6.17-6.02 (m, 2H), 5.68 (dd, J = 10.4, 2.4 Hz, 1H), 5.41 (d, J = 8.6 Hz, 1H), 4.46 (d, J = 12.6 Hz, 1H), 4.21 (t, J = 8.4 Hz, 2H), 4.15-4.05 (m, 3H), 3.97 (s, 3H), 3.90- 3.80 (m, 2H), 3.25-3.15 (m, 1H), 2.88 (t, J = 12.1 Hz, 1H), 2.15-2.00 (m, 2H), 1.50-1.35 (m, 2H). |
| 142 | 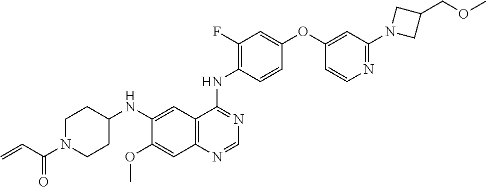 | LC/MS: 614.1 [M + H]⁺. ¹H NMR (400 MHz,) δ 9.23 (s, 1H), 8.23 (s, 1H), 8.00 (d, J = 5.8 Hz, 1H), 7.58 (t, J = 8.8 Hz, 1H), 7.27 (s, 1H), 7.20 (dd, J = 10.9, 2.7 Hz, 1H), 7.02 (s, 1H), 6.99-6.96 (m, 1H), 6.87- 6.83 (m, 1H), 6.22 (dd, J = 5.8, 2.2 Hz, 1H), 6.08 (dd, J = 16.7, 2.5 Hz, 1H), 5.89 (d, J = 2.2 Hz, 1H), 5.65 (dd, J = 10.5, 2.5 Hz, 1H), 5.32 (d, J = 8.5 Hz, 1H), 4.49-4.41 (m, 1H), 4.17- 4.11 (m, 1H), 3.95-3.89 (m, 5H), 3.78-3.66 (m, 1H), 3.61-3.56 (m, 2H), 3.47 (d, J = 6.5 Hz, 2H), 3.23 (s, 3H), 3.20-3.14 (m, 1H), 2.91- |
| | | 2.78 (m, 2H), 2.08-1.99 (m, 2H), 1.45-1.36 |
| | | (m, 2H). |
| 143 | 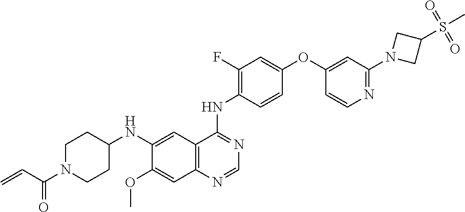 | LC/MS: 648.0 [M + H]⁺. ¹H NMR (400 MHz, DMSO) δ 9.19 (s, 1H), 8.26- 8.22 (m, 2H), 8.07 (d, J = 5.8 Hz, 1H), 7.60 (t, J = 8.7 Hz, 1H), 7.27-7.22 (m, 2H), 7.09-7.04 (m, 2H), 6.90-6.83 (m, 1H), 6.35 (dd, J = 5.7, 2.1 Hz, 1H), 6.14-6.09 (m, 1H), 6.05 (d, J = 2.1 Hz, 1H), 5.69 (dd, J = 10.4, 2.5 Hz, 1H), 5.36- 5.33 (m, 1H), 4.48-4.39 (m, 3H), 4.22 (t, J = 8.8 Hz, 2H), 4.16-4.10 (m, 3H), 3.96 (s, 3H), 3.77-3.73 (m, 1H), 3.05 (s, 3H), 2.88-2.83 (m, 1H), 2.12-2.04 (m, 2H), 1.48-1.41 (m, 2H). |
| 144 | 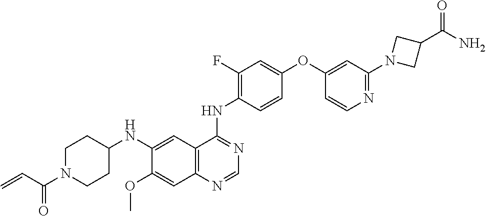 | LC/MS: 613.3 [M + H]⁺. ¹H NMR (400 MHz, DMSO) δ 9.36 (s, 1H), 8.27 (s, 1H), 8.03 (d, J = 5.2 Hz, 1H), 7.59 (t, J = 8.8 Hz, 1H), 7.48 (d, J = 7.6 Hz, 2H), 7.29 (s, 2H), 7.24 (d, J = 10.8 Hz, 1H),7.05 (d, J = 11.2 Hz, 2H), 6.86 (dd, J = 16.4, 10.8 Hz, 1H), 6.29 (d, J = 4.4 Hz, 1H), 6.12 (d, J = 16.8 Hz, 1H), 5.93 (s, 1H), 5.69 (d, J = 10.4 Hz, 1H), 5.40 (d, J = 8.0 Hz, 1H), 4.46 (d, J = 11.2 Hz, 1H), 4.13 (d, J = 12.4 Hz, 1H), 4.12-4.01 (m, 2H), 3.91 (t, J = 8.0 Hz, 2H), 3.81-3.72 (m, 1H), 3.68-3.53 (m, 1H), 3.22-3.11 (m, 1H), 2.86 (t, J = 12.2 Hz, 1H), 2.17-2.03 (m, 2H), 1.51-1.39 (m, 2H), 1.27-1.21 (m, 3H). |
| 145 | 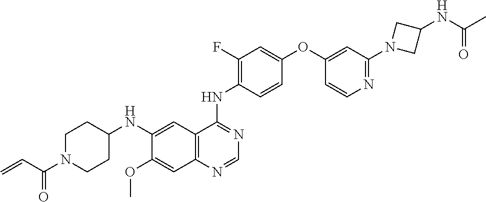 | LC/MS: 627.1 [M + H]⁺. ¹H NMR (400 MHz, DMSO) δ = 9.13 (s, 1H), 8.67 (s, 1H), 8.31 (d, J = 6.4 Hz, 1H), 8.00 (d, J = 7.2 Hz, 1H), 7.75 (t, J = 8.8 Hz, 1H), 7.58- 7.51 (m, 1H), 7.47 (s, 1H), 7.30-7.26 (m, 1H), 7.19 (s, 1H), 6.87 (dd, J = 16.8, 10.4 Hz, 1H), 6.78 (dd, J = 7.6, 2.8 Hz, 1H), 6.28 (d, J = 2.8 Hz, 1H), 6.13 (dd, J = 16.8, 2.4 Hz, 1H), 6.05- 5.97 (m, 1H), 5.70 (dd, J = 10.4, 2.4 Hz, 1H), 4.52-4.28 (m, 5H), 4.22-4.12 (m, 3H), 4.04 (s, 3H), 3.83-3.73 (m, 2H), 2.09-1.80 (m, 4H), 1.62-1.40 (m, 3H). |
| 146 | 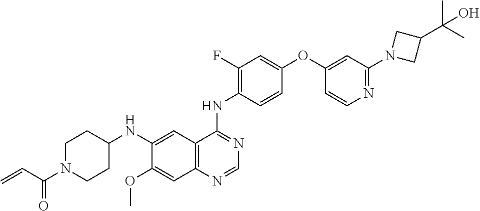 | LC/MS: 628.3 [M + H]⁺. ¹H NMR (400 MHz, DMSO) δ 9.22 (s, 1H), 8.37 (s, 1H), 8.22 (s, 1H), 8.00 (d, J = 5.6 Hz, 1H), 7.58 (t, J = 8.8 Hz, 1H), 7.27 (s, 1H), 7.22 (d, J = 10.4 Hz, 1H), 7.08 (s, 1H), 7.04 (d, J = 8.0 Hz, 1H), 6.86 (dd, J = 16.4, 10.4 Hz, 1H), 6.25 (s, 1H), 6.12 (d, J = 16.8 Hz, 1H), 5.84 (s, 1H), 5.69 (d, J = 10.0 Hz, 1H), 5.34 (d, J = 8.4 Hz, 1H), 4.46 (d, J = 12.0 Hz, 1H), 4.17-4.09 (m, 1H), 3.96 (s, 3H), 3.80 (d, J = 6.8 Hz, 4H), 3.76-3.73 (m, 1H), 3.27-3.18 (m, 2H), 2.90-2.81 (m, 1H), 2.68 (s, 1H), 2.71-2.64 (m, 2H), 1.48- 1.38 (m, 2H), 1.05 (s, 6H). |
| 147 | 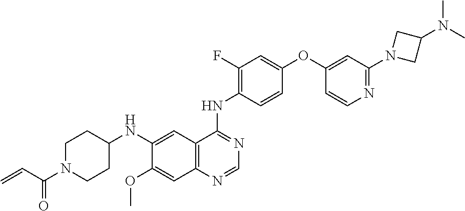 | LC/MS: 613.0 [M + H]⁺. NMR (400 MHz,) δ 9.20 (s, 1H), 8.23 (s, 1H), 8.13 (s, 1H), 8.02 (d, J = 5.6 Hz, 1H), 7.58 (t, J = 8.8 Hz, 1H), 7.26 (s, 1H), 7.22 (dd, J = 10.8, 2.8 Hz, 1H), 7.08 (s, 1H), 7.04 (ddd, J = 8.8, 2.4, 0.8 Hz, 1H), 6.86 (dd, J = 16.8, 10.4 Hz, 1H), 6.29 (dd, J = 5.6, 2.0 Hz, 1H), 6.11 (dd, J = 16.4, 2.4 Hz, 1H), 5.92 (d, J = 2.0 Hz, 1H), 5.68 (dd, J = 10.4, 2.4 Hz, 1H), 5.36-5.30 (m, 1H), 4.51-4.42 (m, 1H), 4.18-4.09 (m, 1H), 3.99-3.94 (m, 5H), 3.76-3.67 (m, 3H), 3.30- 3.28 (m, 1H), 3.21 (d, J = 12.4 Hz, 1H), 2.85 (t, J = 11.6 Hz, 1H), 2.21-2.18 (m, 6H), 2.09- 2.02 (m, 2H), 1.48-1.38 (m, 2H). |
| 148 | 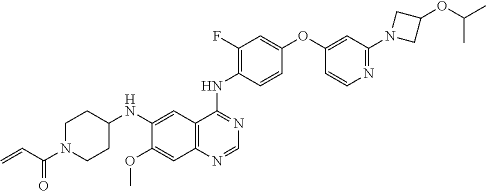 | LC/MS: 628.1 [M + H]⁺. ¹H NMR (400 MHz, DMSO) δ 9.21 (s, 1H), 8.23 (s, 1H), 8.02 (d, J = 5.6 Hz, 1H), 7.58 (t, J = 8.8 Hz, 1H), 7.26 (s, 1H), 7.22 (dd, J = 10.8, 2.4 Hz, 1H), 7.08 (s, 1H), 7.04 (dd, J = 8.8, 1.6 Hz, 1H), 6.86 (dd, J = 16.8, 10.4 Hz, 1H), 6.29 (dd, J = 5.6, 2.0 Hz, 1H), 6.12 (dd, J = 16.8, 2.4 Hz, 1H), 5.93 (d, J = 2.0 Hz, 1H), 5.69 (dd, J = 10.4, 2.4 Hz, 1H), 5.36 (d, J = 8.8 Hz, 1H), 4.47 (dd, J = 11.2, 4.8 Hz, 2H), 4.13 (d, J = 6.4 Hz, 2H), 3.96 (s, 2H), 3.70-3.61 (m, 4H), 3.22 (t, J = 12.0 Hz, 1H), 2.85 (t, J = 12.0 Hz, 1H), 2.13-2.04 (m, |
| | | 2H), 1.52-1.41 (m, 2H), 1.29-1.18 (m, 2H), |
| | | 1.10 (d, J = 6.4 Hz, 6H). |
| 149 | 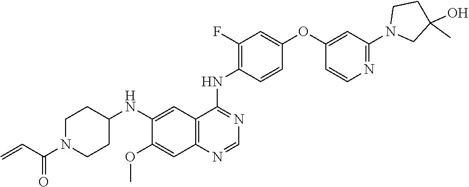 | LC/MS: 614.3 [M + H]⁺. ¹H NMR (400 MHz, DMSO) δ 10.86 (brs, 1H), 8.57 (s, 1H), 8.02 (d, J = 6.2 Hz, 1H), 7.76-7.51 (m, 2H), 7.35-7.32 (m, 1H), 7.23-7.07 (m, 3H), 6.86 (dd, J = 16.7, 10.5 Hz, 1H), 6.32 (d, J = 5.5 Hz, 1H), 6.16-6.07 (m, 2H), 5.83 (d, J = 8.7 Hz, 1H), 5.69 (dd, J = 10.4, 2.4 Hz, 1H), 4.94-4.91 (m, 1H), 4.47 (d, J = 12.3 Hz, 1H), 4.13 (d, J = 11.9 Hz, 1H), 4.02 (s, 3H), 3.94- 3.85 (m, 2H), 3.54-3.46 (m, 2H), 3.29-3.26 (m, 1H), 2.88 (t, J = 11.8 Hz, 1H), 2.09-2.01 (m, 2H), 1.99-1.85 (m, 2H), 1.50-1.41 (m, |
| | | 2H), 1.35 (s, 3H). |
| 152 | 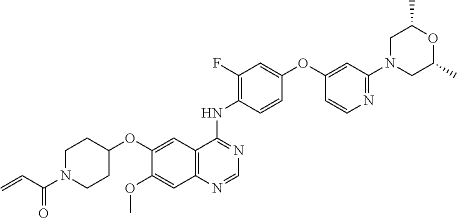 | LC/MS: 629.3 [M + H]⁺. 1H NMR (400 MHz, DMSO) δ 9.61 (s, 1H), 8.39 (s, 1H), 8.07 (d, J = 5.7 Hz, 1H), 7.93 (s, 1H), 7.57 (t, J = 8.8 Hz, 1H), 7.23-7.20 (m, 2H), 7.02 (dd, J = 8.6, 1.8 Hz, 1H), 6.85 (dd, J = 16.7, 10.5 Hz, 1H), 6.51 (d, J = 1.9 Hz, 1H), 6.27 (dd, J = 5.7, 2.0 Hz, 1H), 6.12 (dd, J = 16.7, 2.4 Hz, 1H), 5.69 (dd, J = 10.5, 2.4 Hz, 1H), 4.82-4.75 (m, 1H), 4.12 (d, J = 11.3 Hz, 2H), 4.14-3.87 (m, 5H), 3.65-3.55 (m, 2H), 3.53-3.39 (m, 2H), 2.39 (dd, J = 12.7, 10.7 Hz, 2H), 2.07- 2.02 (m, 2H), 1.72-1.66 (m, 2H), 1.14 (d, J = 6.2 Hz, 6H). |
| 153 | 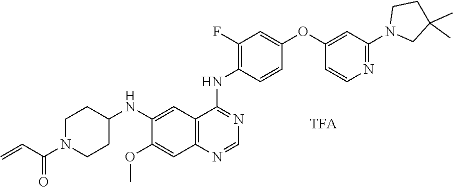 | LC/MS: 612.2 [M + H]⁺. 1H NMR (400 MHz, DMSO-d6) δ 10.87 (s, 1H), 8.71 (s, 1H), 8.05 (d, J = 7.0 Hz, 1H), 7.72 (t, J = 8.7 Hz, 1H), 7.50 (dd, J = 12.0, 7.0, 2.5 Hz, 1H), 7.48 (s, 1H), 7.26 (dd, J = 8.7, 1.8 Hz, 1H), 7.22 (s, 1H), 6.87 (dd, J = 16.7, 10.5 Hz, 1H), 6.54 (d, J = 5.1 Hz, 1H), 6.33 (s, 1H), 6.13 (dd, J = 16.7, 2.4 Hz, 1H), 6.09-6.02 (m, 1H), 5.70 (dd, J = 10.4, 2.4 Hz, 1H), 4.48 (d, J = 11.9 Hz, 1H), 4.16 (d, J = 12.2 Hz, 1H), 4.05 (s, 3H), 3.60- 3.52 (m, 4H), 3.27-3.17 (m, 3H), 2.84 (t, J = 12.1 Hz, 1H), 2.04 (d, J = 10.9 Hz, 2H), 1.83 (t, |
| | | J = 7.0 Hz, 2H), 1.53-1.46 (m, 2H), 1.11 (d, |
| | | J = 4.6 Hz, 6H). |
| 154 | 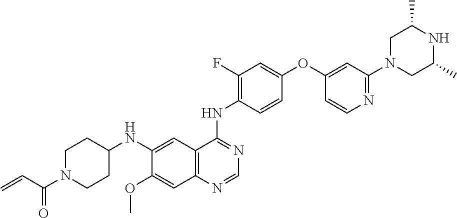 | LC/MS: 627.1 [M + H]⁺. ¹H NMR (400 MHz,) δ 8.65 (s, 1H), 8.60-8.49 (m, 1H), 8.10 (d, J = 5.6 Hz, 1H), 7.59 (t, J = 8.8 Hz, 1H), 7.46 (s, 1H), 7.27 (dd, J = 10.8, 2.8 Hz, 1H), 7.16 (s, 1H), 7.08-7.04 (m, 1H), 6.83 (dd, J = 16.8, 10.4 Hz, 1H), 6.73 (d, J = 2.0 Hz, 1H), 6.30 (dd, J = 6.0, 2.0 Hz, 1H), 6.09 (dd, J = 16.8, 2.4 Hz, 1H), 6.02-5.94 (m, 1H), 5.66 (dd, J = 10.4, 2.4 Hz, 1H), 4.44 (d, J = 12.0 Hz, 3H), 4.15-4.10 (m, 1H), 4.00 (s, 3H), 3.78-3.70 (m, 1H), 3.32-3.14 (m, 3H), 2.83-2.70 (m, 3H), 2.00 (d, J = 11.6 Hz, 2H), 1.52-1.39 (m, 2H), 1.25 (s, 3H), 1.23 (s, 3H), 1.22-1.18 (m, 1H). |
| 155 | 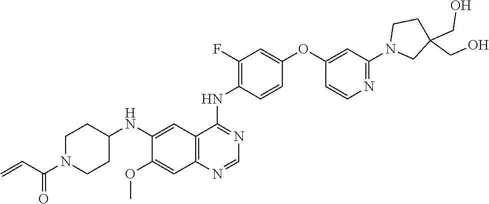 | LC/MS: 644.3 [M + H]⁺. ¹H NMR (400 MHz, DMSO) δ 9.19 (s, 1H), 8.23- 8.20 (m, 2H), 8.01 (d, J = 5.7 Hz, 1H), 7.58 (t, J = 8.8 Hz, 1H), 7.30-7.18 (m, 2H), 7.13-7.01 (m, 2H), 6.86 (dd, J = 16.7, 10.5 Hz, 1H), 6.22 (dd, J = 5.7, 2.0 Hz, 1H), 6.12 (dd, J = 16.7, 2.4 Hz, 1H), 5.89 (d, J = 1.8 Hz, 1H), 5.69 (dd, J = 10.4, 2.4 Hz, 1H), 5.34 (d, J = 8.4 Hz, 1H), 4.46 (d, J = 12.6 Hz, 1H), 4.14 (d, J = 13.2 Hz, 1H), 3.96 (s, 3H), 3.78-3.71 (d, J = 8.8 Hz, 1H), 3.47- 3.30 (m, 6H), 3.23 (t, J = 12.1 Hz, 1H), 3.15 (s, 2H), 2.85 (t, J = 11.7 Hz, 1H), 2.14-2.00 (m, 2H), 1.78 (t, J = 7.1 Hz, 2H), 1.51-1.36 (m, 2H). |
| 156 | 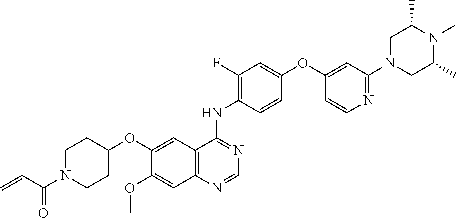 | LC/MS: 642.3 [M + H]⁺. ¹H NMR (400 MHz, DMSO) δ 9.47 (s, 1H), 8.36 (s, 1H), 8.08 (d, J = 5.5 Hz, 1H), 7.90 (s, 1H), 7.58 (t, J = 8.8 Hz, 1H), 7.24-7.17 (m, 2H), 7.02 (dd, J = 8.7, 1.9 Hz, 1H), 6.86 (dd, J = 16.7, 10.5 Hz, 1H), 6.75-6.51 (brs, 1H), 6.26 (s, 1H), 6.12 (dd, J = 16.7, 2.4 Hz, 1H), 5.69 (dd, J = 10.5, 2.4 Hz, 1H), 4.82-4.73 (m, 1H), 4.51- 4.08 (brs, 2H), 3.97-3.81 (m, 5H), 3.57-3.42 (m, 2H), 2.75-2.53 (m, 2H), 2.48-1.92 (m, 6H), 1.73-1.66 (m, 2H), 1.35-1.06 (m, 7H). |
| 157 | 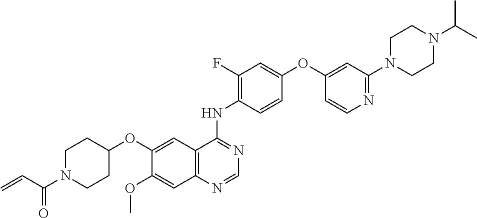 | LC/MS: 642.2 [M + H]⁺. ¹H NMR (400 MHz, DMSO) δ 9.47 (s, 1H), 8.36 (s, 1H), 8.07 (d, J = 5.6 Hz, 1H), 7.90 (s, 1H), 7.58 (t, J = 8.8 Hz, 1H), 7.25-7.19 (m, 2H), 7.04 (d, J = 8.4 Hz, 1H), 6.86 (dd, J = 16.7, 10.4 Hz, 1H), 6.44 (s, 1H), 6.26 (d, J = 4.5 Hz, 1H), 6.12 (dd, J = 16.7, 2.3 Hz, 1H), 5.69 (dd, J = 10.4, 2.4 Hz, 1H), 4.81-4.76 (m, 1H), 3.96- 3.86 (m, 5H), 3.58-3.46 (m, 7H), 2.56-2.54 (m, 4H), 2.07-2.03 (m, 2H), 1.72-1.69 (m, 2H), 1.02 (d, J = 5.2 Hz, 6H). |
| 158 | 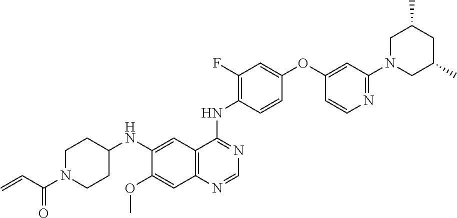 | LC/MS: 626.1 [M + H]⁺. ¹H NMR (400 MHz, DMSO) δ 9.18 (s, 1H), 8.22 (s, 1H), 8.04 (d, J = 5.6 Hz, 1H), 7.57 (t, J = 8.8 Hz, 1H), 7.26 (s, 1H), 7.19 (d, J = 10.8 Hz, 1H), 7.08 (s, 1H), 7.02 (d, J = 7.6 Hz, 1H), 6.86 (dd, J = 16.8, 10.4 Hz, 1H), 6.47 (s, 1H), 6.15 (dd, J = 19.2, 10.4 Hz, 2H), 5.69 (d, J = 10.4 Hz, 1H), 5.33 (d, J = 8.0 Hz, 1H), 4.46 (d, J = 7.6 Hz, 1H), 4.26 (d, J = 11.6 Hz, 2H), 4.14 (d, J = 12.8 Hz, 1H), 3.97 (s, 3H), 3.78-3.74 (m, 1H), 3.21 (t, J = 8.0 Hz, 1H), 2.86 (t, J = 11.6 Hz, 1H), 2.27 (t, J = 11.6 Hz, 2H), 2.15-2.07 (m, 2H), 1.78 (d, J = 12.4 Hz, 1H), 1.63-1.51 (m, 5H), 0.89 (d, J = 6.4 Hz, 6H). |
| 159 | 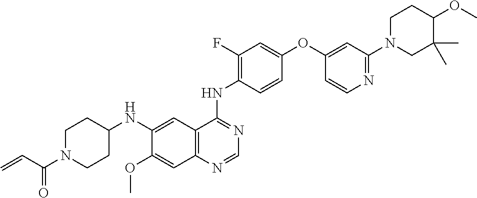 | LC/MS: 656.1 [M + H]⁺. ¹H NMR (400 MHz, DMSO) δ 9.17 (s, 1H), 8.22 (s, 1H), 8.01 (d, J = 5.6 Hz, 1H), 7.56 (t, J = 8.8 Hz, 1H), 7.25 (s, 1H), 7.19 (dd, J = 11.2, 2.8 Hz, 1H), 7.08 (s, 1H), 7.01 (dd, J = 8.4, 2.0 Hz, 1H), 6.86 (dd, J = 16.8, 10.4 Hz, 1H), 6.46 (d, J = 2.0 Hz, 1H), 6.17-6.04 (m, 2H), 5.68 (dd, J = 10.4, 2.4 Hz, 1H), 5.33 (d, J = 8.4 Hz, 1H), 4.46 (d, J = 11.2 Hz, 1H), 4.13 (d, J = 12.8 Hz, 1H), 3.96 (s, 3H), 3.85-3.73 (m, 2H), 3.30 (s, 3H), 3.23 (m, 1H), 3.10-3.05 (m, 1H), 2.98 (dd, J = 9.2, 4.0 Hz, 1H), 2.95-2.81 (m, 2H), 2.13-2.04 (m, 2H), 2.01-1.92 (m, 1H), 1.90-1.78 (m, 1H), |
| | | 1.51-1.39 (m, 3H), 0.94 (s, 3H), 0.84 (s, 3H). |
| 160 | 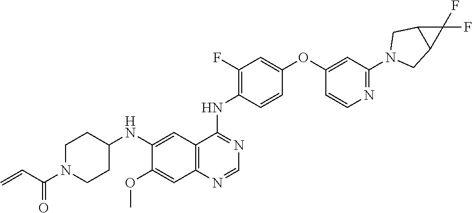 | LC/MS: 632.0 [M + H]⁺. ¹H NMR (400 MHz, DMSO) δ 9.18 (s, 1H), 8.22 (s, 1H), 8.03 (d, J = 5.6 Hz, 1H), 7.58 (t, J = 8.8 Hz, 1H), 7.26 (s, 1H), 7.22 (dd, J = 10.8, 2.8 Hz, 1H), 7.08 (s, 1H), 7.04 (dd, J = 8.8, 2.0 Hz, 1H), 6.86 (dd, J = 16.8, 10.4 Hz, 1H), 6.24 (dd, J = 5.6, 2.0 Hz, 1H), 6.12 (dd, J = 16.8, 2.4 Hz, 1H), 6.07 (d, J = 2.0 Hz, 1H), 5.69 (dd, J = 10.4, 2.4 Hz, 1H), 5.34 (d, J = 8.8 Hz, 1H), 4.46 (d, J = 12.8 Hz, 1H), 4.13 (d, J = 13.6 Hz, 1H), 3.96 (s, 3H), 3.69 (dd, J = 34.8, 10.4 Hz, 5H), 3.23 (t, J = 12.4 Hz, 1H), 2.86 (t, J = 11.6 Hz, 1H), 2.67 (d, J = 10.4 Hz, 2H), 2.15-2.03 (m, 2H), 1.52- 1.40 (m, 2H). |
| 161 | 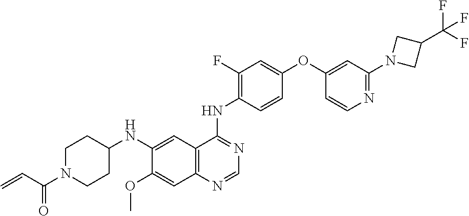 | LC/MS: 637.8 [M + H]⁺. 1H NMR (400 MHz, DMSO-d6) δ 10.79 (s, 1H), 8.71 (s, 1H), 8.10 (d, J = 6.1 Hz, 1H), 7.66 (t, J = 8.7 Hz, 1H), 7.45 (s, 1H), 7.40 (dd, J = 10.8, 2.6 Hz, 1H), 7.20-7.15 (m, 2H), 6.87 (dd, J = 16.7, 10.5 Hz, 1H), 6.43 (d, J = 4.1 Hz, 1H), 6.17 (d, J = 1.9 Hz, 1H), 6.13 (dd, J = 16.7, 2.4 Hz, 1H), 6.05-6.01 (m, 1H), 5.70 (dd, J = 10.5, 2.4 Hz, 1H), 4.49 (d, J = 12.6 Hz, 1H), 4.24 (t, J = 8.9 Hz, 2H), 4.16 (d, J = 13.6 Hz, 1H), 4.06- 4.00 (m, 5H), 3.81-3.74 (m, 2H), 3.22-3.19 (m, 1H), 2.84 (t, J = 12.7 Hz, 1H), 2.04 (d, J = 10.8 Hz, 2H), 1.62-1.47 (m, 2H). |
| 162 | 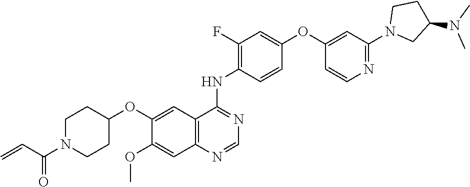 | LC/MS: 628.0 [M + H]⁺. ¹H NMR (400 MHz, DMSO) δ 11.09 (s, 1H), 10.27 (s, 1H), 8.82 (s, 1H), 8.12 (d, J = 6.0 Hz, 2H), 7.67 (t, J = 8.8 Hz, 1H), 7.43-7.35 (m, 2H), 7.17 (dd, J = 8.8, 1.6 Hz, 1H), 6.86 (dd, J = 16.8, 10.4 Hz, 1H), 6.45-6.36 (m, 2H), 6.13 (dd, J = 16.8, 2.4 Hz, 1H), 5.70 (dd, J = 10.4, 2.4 Hz, 1H), 4.85 (d, J = 3.6 Hz, 1H), 4.05-3.98 (m, 3H), 3.95-3.88 (m, 4H), 3.65-3.60 (m, 4H), 3.55-3.43 (m, 1H), 2.87 (s, 6H), 2.25- 2.19 (m, 1H), 2.1-61.96 (m, 3H), 1.85-1.70 (m, 2H). |
| 163 | 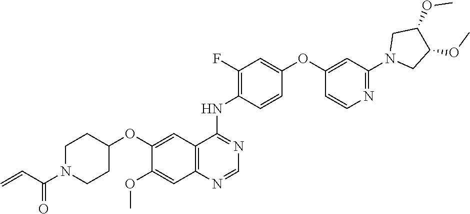 | LC/MS: 644.9 [M + H]⁺. ¹H NMR (400 MHz, DMSO) δ 11.02 (brs, 1H), 8.81 (s, 1H), 8.14-8.03 (m, 2H), 7.70 (t, J = 8.7 Hz, 1H), 7.47 (d, J = 11.2 Hz, 1H), 7.35-7.33 (m, 1H), 7.23 (d, J = 8.7 Hz, 1H), 6.89-6.82 (m, 1H), 6.52-6.38 (m, 2H), 6.13 (dd, J = 16.7, 2.4 Hz, 1H), 5.70 (dd, J = 10.5, 2.4 Hz, 1H), 4.89-4.82 (m, 1H), 4.13-4.06 (m, 2H), 4.02 (s, 3H), 3.97-3.82 (m, 3H), 3.68-3.60 (m, 2H), 3.57-3.43 (m, 4H), 3.37-3.34 (m, 5H), 2.10- 2.06 (m, 2H), 1.74 (d, J = 14.5 Hz, 2H). |
| 164 | 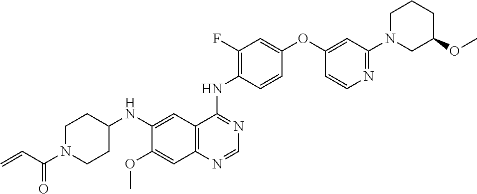 | LC/MS: 628.3 [M + H]⁺. ¹H NMR (400 MHz, DMSO) δ 9.18 (s, 1H), 8.22 (s, 1H), 8.04 (d, J = 5.7 Hz, 1H), 7.57 (t, J = 8.8 Hz, 1H), 7.27-7.18 (m, 2H), 7.09-7.00 (m, 2H), 6.86 (dd, J = 16.7, 10.5 Hz, 1H), 6.42 (d, J = 1.9 Hz, 1H), 6.21 (dd, J = 5.7, 2.0 Hz, 1H), 6.12 (dd, J = 16.7, 2.4 Hz, 1H), 5.69 (dd, J = 10.5, 2.4 Hz, 1H), 5.34 (d, J = 8.6 Hz, 1H), 4.46 (d, J = 12.4 Hz, 1H), 4.14 (d, J = 12.4 Hz, 1H), 4.06-3.92 (m, 4H), 3.80-3.70 (m, 2H), 3.27 (s, 3H), 3.26-3.07 (m, 5H), 2.85 (t, J = 11.9 Hz, 1H), 2.15-2.05 (m, 2H), 2.00-1.90 (m, 1H), 1.75-1.65 (m, 1H), 1.50-1.45 (m, 2H). |
| 166 | 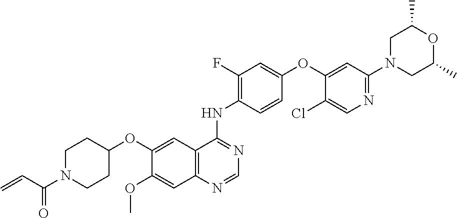 | LC/MS: 663.3 [M + H]⁺. ¹H NMR (400 MHz, DMSO) δ 8.37 (s, 1H), 8.24 (s, 1H), 7.94 (s, 1H), 7.56 (t, J = 8.8 Hz, 1H), 7.23-7.17 (m, 2H), 6.96 (dd, J = 8.8, 2.3 Hz, 1H), 6.89-6.82 (m, 1H), 6.55 (s, 1H), 6.12 (dd, J = 16.7, 2.4 Hz, 1H), 5.69 (dd, J = 10.5, 2.4 Hz, 1H), 4.85-4.75 (m, 1H), 4.03 (d, J = 11.2 Hz, 2H), 3.95-3.85 (m, 5H), 3.61-3.39 (m, 5H), 2.39 (dd, J = 12.7, 10.7 Hz, 2H), 2.10-2.00 (m, 2H), 1.75-1.65 (m, 2H), 1.12 (d, J = 6.2 Hz, 6H). |
| 168 | 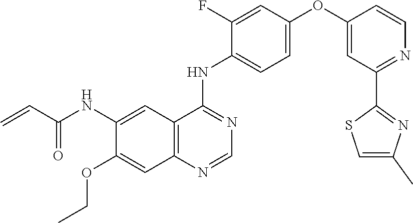 [SMILES: C=CC(=O)Nc1cc2c(Nc3ccc(Oc4ccnc(-c5nc(C)cs5)c4)cc3F)ncnc2cc1OCC] | LC/MS: 543.1 [M + H]⁺. ¹H NMR (400 MHz, DMSO) δ 9.80 (s, 1H), 9.63 (s, 1H), 8.94 (s, 1H), 8.56 (d, J = 5.7 Hz, 1H), 8.43 (s, 1H), 7.64 (t, J = 8.8 Hz, 1H), 7.52 (d, J = 2.4 Hz, 1H), 7.45 (d, J = 0.9 Hz, 1H), 7.39 (dd, J = 10.8, 2.7 Hz, 1H), 7.28 (s, 1H), 7.21-7.15 (m, 2H), 6.77 (dd, J = 17.0, 10.3 Hz, 1H), 6.33 (dd, J = 17.0, 1.9 Hz, 1H), 5.82 (dd, J = 10.2, 1.9 Hz, 1H), 4.31 (q, J = 7.0 Hz, 2H), 2.42 (d, J = 0.8 Hz, 3H), 1.47 (t, J = 7.0 Hz, 3H). |
| 169 | 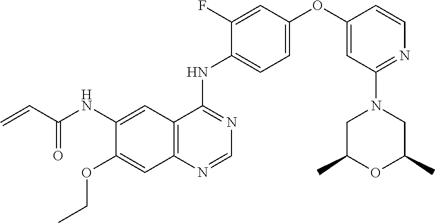 [SMILES: C=CC(=O)Nc1cc2c(Nc3ccc(Oc4ccnc(N5C[C@@H](C)O[C@@H](C)C5)c4)cc3F)ncnc2cc1OCC] | LC/MS: 559.0 [M + H]⁺. ¹H NMR (400 MHz,) δ 9.73-9.49 (m, 2H), 8.88 (s, 1H), 8.35 (s, 1H), 8.03 (d, J = 5.7 Hz, 1H), 7.49 (t, J = 8.8 Hz, 1H), 7.24-6.66 (m, 4H), 6.49-6.20 (m, 3H), 5.81-5.75 (m, 1H), 4.27 (q, J = 7.0 Hz, 2H), 4.12-4.04 (m, 2H), 3.60- 3.50 (m, 2H), 2.40-2.31 (m, 2H), 1.43 (t, J = 7.0 Hz, 3H), 1.11 (d, J = 6.2 Hz, 6H). |
| 170 | 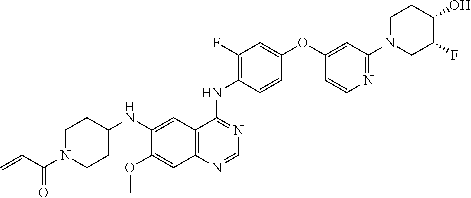 | LC/MS: 631.8 [M + H]⁺. ¹H NMR (400 MHz, DMSO) δ 9.39 (brs, 1H), 8.28 (s, 1H), 8.04 (d, J = 5.7 Hz, 1H), 7.58 (t, J = 8.8 Hz, 1H), 7.29 (s, 1H), 7.22 (dd, J = 11.0, 2.7 Hz, 1H), 7.09 (s, 1H), 7.03 (dd, J = 8.6, 1.9 Hz, 1H), 6.86 (dd, J = 16.7, 10.5 Hz, 1H), 6.48 (d, J = 1.9 Hz, 1H), 6.23 (dd, J = 5.7, 1.9 Hz, 1H), 6.12 (dd, J = 16.7, 2.5 Hz, 1H), 5.69 (dd, J = 10.4, 2.5 Hz, 1H), 5.42 (d, J = 8.5 Hz, 1H), 5.10 (d, J = 4.6 Hz, 1H), 4.73-4.57 (m, 1H), 4.46 (d, J = 12.8 Hz, 1H), 4.24-4.10 (m, 2H), 4.01-3.92 (m, 4H), 3.86-3.71 (m, 2H), 3.42- 3.38 (m, 1H), 3.24 (d, J = 13.1 Hz, 1H), 3.14- |
| | | 3.07 (m, 1H), 2.86 (t, J = 11.8 Hz, 1H), 2.12- |
| | | 2.02 (m, 2H), 1.73-1.62 (m, 2H), 1.51-1.39 |
| | | (m, 2H). |
| 171 | 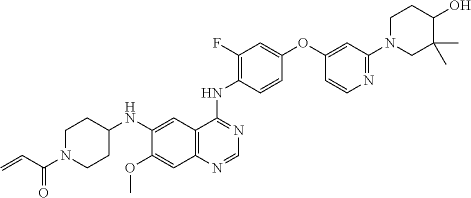 | LC/MS: 642.1 [M + H]⁺. ¹H NMR (400 MHz, DMSO) δ 9.19 (s, 1H), 8.37 (s, 1H), 8.21 (s, 1H), 8.00 (d, J = 5.6 Hz, 1H), 7.56 (t, J = 8.8 Hz, 1H), 7.26 (s, 1H), 7.18 (d, J = 11.2 Hz, 1H), 7.07 (s, 1H), 7.01 (d, J = 7.6 Hz, 1H), 6.86 (dd, J = 16.8, 10.4 Hz, 1H), 6.43 (s, 1H), 6.17-6.07 (m, 2H), 5.68 (d, J = 12.0 Hz, 1H), 5.33 (d, J = 8.4 Hz, 1H), 4.45 (d, J = 14.0 Hz, 1H), 4.14 (d, J = 17.6 Hz, 1H), 4.03-3.94 (m, 4H), 3.79-3.72 (m, 2H), 3.60-3.52 (m, 1H), 3.30-3.26 (m, 2H), 3.07-2.98 (m, 1H), 2.89-2.85 (m, 1H), 2.77 (d, J = 12.8 Hz, 1H), 2.14-2.03 (m, 2H), 1.71-1.64 (m, 1H), 1.51- |
| | | 1.40 (m, 3H), 0.91 (s, 3H), 0.79 (s, 3H). |
| 172 | 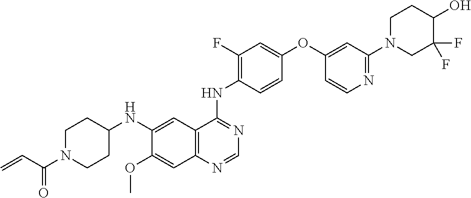 | LC/MS: 650.0 [M + H]⁺. NMR (400 MHz,) δ 9.20 (s, 1H), 8.31 (s, 1H), 8.22 (s, 1H), 8.06 (d, J = 5.6 Hz, 1H), 7.58 (t, J = 8.8 Hz, 1H), 7.26 (s, 1H), 7.21 (dd, J = 11.2, 2.8 Hz, 1H), 7.08 (s, 1H), 7.05-7.01 (m, 1H), 6.86 (dd, J = 16.8, 10.4 Hz, 1H), 6.59 (d, J = 2.0 Hz, 1H), 6.26 (dd, J = 5.6, 2.0 Hz, 1H), 6.12 (dd, J = 16.8, 2.4 Hz, 1H), 5.69 (dd, J = 10.4, 2.4 Hz, 1H), 5.33 (d, J = 8.4 Hz, 1H), 4.46 (d, J = 12.0 Hz, 1H), 4.15-4.05 (m, 2H), 3.96 (s, 3H), 3.92- 3.85 (m, 1H), 3.80-3.69 (m, 4H), 3.26-3.20 (m, 2H), 2.86 (t, J = 11.6 Hz, 1H), 2.11-2.04 (m, 2H), 1.89-1.82 (m, 1H), 1.70-1.63 (m, |
| | | 1H), 1.47-1.40 (m, 2H). |
| 173 | 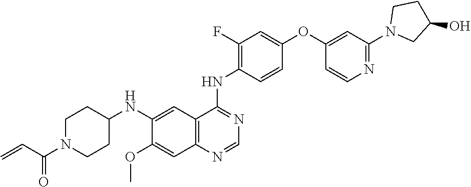 | LC/MS: 600.3 [M + H]⁺. ¹H NMR (400 MHz, DMSO) δ 10.87 (s, 1H), 8.70 (s, 1H), 8.05 (d, J = 6.8 Hz, 1H), 7.71 (t, J = 8.8 Hz, 1H), 7.49 (d, J = 10.8 Hz, 2H), 7.32- 7.18 (m, 2H), 6.87 (dd, J = 16.8, 10.4 Hz, 1H), 6.52 (s, 1H), 6.37 (s, 1H), 6.13 (dd, J = 16.8, 2.4 Hz, 1H), 6.02 (d, J = 9.2 Hz, 1H), 5.70 (dd, J = 10.4, 2.4 Hz, 1H), 4.48 (d, J = 17.2 Hz, 2H), 4.16 (d, J = 13.6 Hz, 1H), 4.04 (s, 3H), 3.81- 3.75 (m, 1H), 3.54 (d, J = 3.6 Hz, 4H), 3.35 (d, J = 10.8 Hz, 1H), 3.22 (t, J = 12.4 Hz, 1H), 2.84 (t, J = 11.6 Hz, 1H), 2.09-1.95 (m, 4H), 1.54- |
| | | 1.42 (m, 2H). |
| 174 | 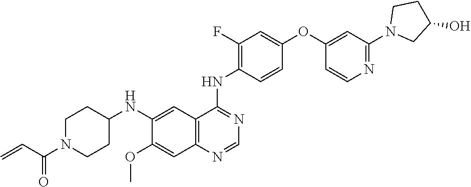 | LC/MS: 600.1 [M + H]⁺. ¹H NMR (400 MHz,) δ 10.86 (s, 1H), 8.70 (s, 1H), 8.06 (d, J = 6.8 Hz, 1H), 7.71 (t, J = 8.7 Hz, 1H), 7.53-7.47 (m, 2H), 7.28-7.22 (m, 2H), 6.87 (dd, J = 16.7, 10.5 Hz, 1H), 6.51 (d, J = 6.6 Hz, 1H), 6.36 (s, 1H), 6.13 (dd, J = 16.7, 2.4 Hz, 1H), 6.03 (d, J = 7.8 Hz, 1H), 5.70 (dd, J = 10.4, 2.4 Hz, 1H), 4.48 (d, J = 17.6 Hz, 2H), 4.16 (d, J = 12.7 Hz, 1H), 4.04 (s, 3H), 3.82-3.76 (m, 2H), 3.56-3.52 (m, 3H), 3.35 (d, J = 10.3 Hz, 1H), 3.22 (t, J = 12.2 Hz, 1H), 2.87-2.80 (m, 1H), 2.11-2.04 (m, 4H), 1.55-1.44 (m, 2H). |
| 175 | 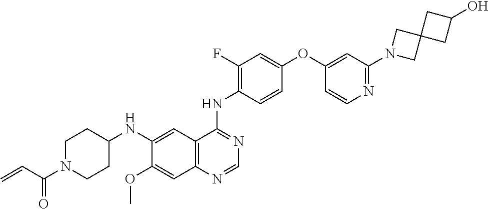 | LC/MS: 626.3 [M + H]⁺. ¹H NMR (400 MHz, DMSO) δ 10.97 (s, 1H), 8.72 (s, 1H), 8.06 (d, J = 7.0 Hz, 1H), 7.72 (t, J = 8.7 Hz, 1H), 7.50 (dd, J = 10.4, 2.7 Hz, 2H), 7.26 (dd, J = 12.1, 3.5 Hz, 2H), 6.87 (dd, J = 16.7, 10.5 Hz, 1H), 6.57 (d, J = 7.0 Hz, 1H), 6.18-6.02 (m, 3H), 5.70 (dd, J = 10.4, 2.4 Hz, 1H), 4.48 (d, J = 12.8 Hz, 1H), 4.20-4.07 (m, 4H), 4.05 (s, 3H), 4.03-3.97 (m, 1H), 3.83- 3.74 (m, 1H), 3.22 (t, J = 12.4 Hz, 1H), 2.84 (t, J = 11.6 Hz, 1H), 2.54 (s, 2H), 2.49-2.47 (m, 2H), 2.09-1.99 (m, 4H), 1.56-1.42 (m, 2H). |
| 176 | 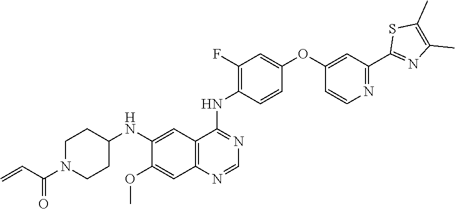 | LC/MS: 626.0 [M + H]⁺. ¹H NMR (400 MHz, DMSO) δ 9.23 (s, 1H), 8.53 (d, J = 5.6 Hz, 1H), 8.25 (s, 1H), 7.66 (t, J = 8.8 Hz, 1H), 7.45-7.39 (m, 2H), 7.27 (s, 1H), 7.20- 7.17 (m, 1H), 7.15 (dd, J = 5.7, 2.5 Hz, 1H), 7.09 (s, 1H), 6.87 (dd, J = 6.2 Hz, 1H), 6.14- 6.09 (m, 1H), 5.69 (dd, J = 10.4, 2.5 Hz, 1H), 5.35 (d, J = 8.6 Hz, 1H), 4.47 (d, J = 12.4 Hz, 1H), 4.14 (d, J = 9.7 Hz, 1H), 3.97 (s, 3H), 3.79- 3.74 (m, 1H), 3.26-3.22 (m, 1H), 2.89-2.85 (m, 1H), 2.39 (s, 3H), 2.30 (s, 3H), 2.07 (s, 2H), 1.47-1.40 (m, 2H). |
| 177 | 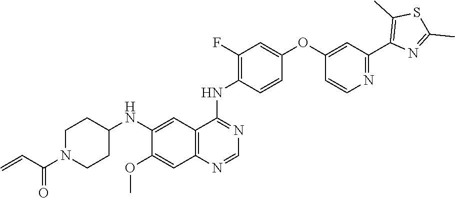 | LC/MS: 626.2 [M + H]⁺. ¹H NMR (400 MHz, DMSO) δ 9.25 (s, 1H), 8.58 (d, J = 5.6 Hz, 1H), 8.25 (s, 1H), 7.64 (t, J = 8.8 Hz, 1H), 7.55 (d, J = 2.4 Hz, 1H), 7.35 (dd, J = 10.8, 2.5 Hz, 1H), 7.27 (s, 1H), 7.14 (d, J = 8.3 Hz, 1H), 7.09 (s, 1H), 7.02 (dd, J = 5.6, 2.5 Hz, 1H), 6.86 (dd, J = 16.6, 10.5 Hz, 1H), 6.12 (dd, J = 16.7, 2.4 Hz, 1H), 5.69 (dd, J = 10.4, 2.4 Hz, 1H), 5.36 (d, J = 8.4 Hz, 1H), 4.46 (d, J = 12.3 Hz, 1H), 4.14 (d, J = 11.7 Hz, 1H), 3.97 (s, 3H), 3.81-3.74 (m, 1H), 3.27-3.19 (m, 1H), 2.88 (d, J = 12.9 Hz, 1H), 2.79 (s, 3H), 2.60 (s, 3H), 2.11-2.03 (m, 2H), 1.51-1.39 (m, 2H). |
| 178 | 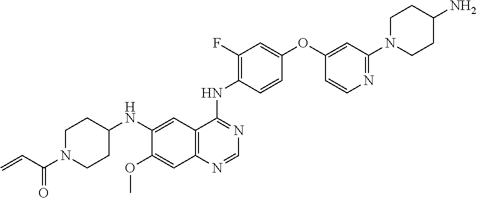 | LC/MS: 612.9 [M + H]⁺. ¹H NMR (400 MHz, DMSO) δ 9.19 (s, 1H), 8.24- 8.21 (m, 2H), 8.07 (d, J = 5.7 Hz, 1H), 7.58 (t, J = 8.8 Hz, 1H), 7.26 (s, 1H), 7.19 (dd, J = 11.0, 2.6 Hz, 1H), 7.08 (s, 1H), 7.02 (dd, J = 8.5, 2.0 Hz, 1H), 6.87 (dd, J = 16.7, 10.5 Hz, 1H), 6.52 (d, J = 1.9 Hz, 1H), 6.24 (dd, J = 5.7, 1.9 Hz, 1H), 6.12 (dd, J = 16.7, 2.5 Hz, 1H), 5.69 (dd, J = 10.4, 2.4 Hz, 1H), 5.35 (d, J = 8.5 Hz, 1H), 4.46 (d, J = 12.8 Hz, 1H), 4.29 (d, J = 13.4 Hz, 2H), 4.14 (d, J = 13.2 Hz, 1H), 3.96 (s, 3H), 3.75 (d, J = 7.9 Hz, 1H), 3.28-3.17 (m, 3H), 2.92- 2.82 (m, 3H), 2.06 (d, J = 10.4 Hz, 2H), 1.89 |
| | | (d, J = 9.1 Hz, 2H), 1.52-1.36 (m, 4H), 1.23 (s, |
| | | 1H). |
| 179 | 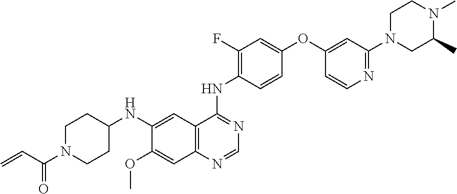 | LC/MS: 627.3 [M + H]⁺. ¹H NMR (400 MHz, DMSO) δ 10.47 (s, 1H), 8.69 (s, 1H), 8.14 (d, J = 6.0 Hz, 1H), 7.69 (s, 1H), 7.62 (t, J = 8.8 Hz, 1H), 7.31 (dd, J = 10.8,2.8 Hz, 1H), 7.20 (s, 1H), 7.10 (d, J = 7.2 Hz, 1H), 6.87 (dd, J = 16.8, 10.4 Hz, 1H), 6.76 (s, 1H), 6.37 (d, J = 4.0 Hz, 1H), 6.12 (d, J = 16.8 Hz, 1H), 6.05-5.99 (m, 1H), 5.69 (d, J = 10.4 Hz, 1H), 4.52-4.39 (m, 3H), 4.18-4.12 (m, 1H), 4.04 (s, 3H), 3.91-3.86 (m, 1H), 3.56- 3.52 (m, 1H), 3.31-3.21 (m, 3H), 3.14-3.08 (m, 1H), 3.03-2.96 (m, 1H), 2.91-2.85 (m, 1H), 2.82 (d, J = 4.4 Hz, 3H), 2.05-1.98 (m, |
| | | 2H), 1.52-1.45 (m, 2H), 1.35 (d, J = 6.4 Hz, |
| | | 3H). |
| 180 | 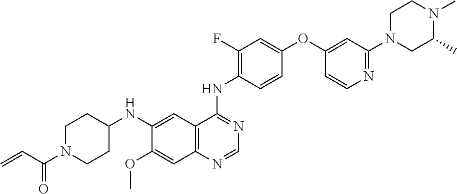 | LC/MS: 627.1 [M + H]⁺. ¹H NMR (400 MHz,) δ 9.17 (s, 1H), 8.18 (s, 1H), 8.10 (s, 1H), 8.03 (d, J = 5.7 Hz, 1H), 7.54 (t, J = 8.8 Hz, 1H), 7.23 (s, 1H), 7.16 (dd, J = 11.0, 2.7 Hz, 1H), 7.04 (s, 1H), 7.02-6.95 (m, 1H), 6.90-6.76 (m, 1H), 6.52-6.44 (m, 1H), 6.22 (dd, J = 5.7, 2.0 Hz, 1H), 6.08 (dd, J = 16.7, 2.5 Hz, 1H), 5.65 (dd, J = 10.4, 2.5 Hz, 1H), 5.30 (d, J = 8.5 Hz, 1H), 4.47-4.37 (m, 1H), 4.13-4.00 (m, 3H), 3.93 (s, 3H), 3.76-3.68 (m, 1H), 3.47-3.40 (m, 1H), 3.21-3.17 (m, 1H), 2.98-2.88 (m, 2H), 2.86-2.77 (m, 1H), 2.66- 2.57 (m, 1H), 2.45-2.31 (m, 4H), 2.09-1.99 |
| | | (m, 2H), 1.47-1.32 (m, 2H), 1.07 (d, J = 6.2 Hz, |
| | | 3H). |
| 182 | 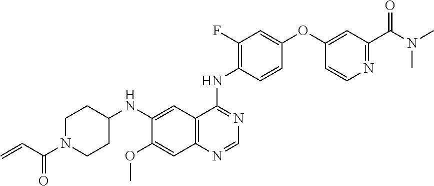 | LC/MS: 586.1 [M + H]⁺. ¹H NMR (400 MHz, DMSO) δ 9.34 (s, 1H), 8.53 (d, J = 5.7 Hz, 1H), 8.29 (s, 1H), 7.65 (t, J = 8.7 Hz, 1H), 7.38 (dd, J = 10.8, 2.6 Hz, 1H), 7.27 (s, 1H), 7.16 (dd, J = 8.7, 1.9 Hz, 1H), 7.13- 7.08 (m, 2H), 7.07 (d, J = 2.2 Hz, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.41 (d, J = 8.9 Hz, 1H), 4.46 (d, J = 12.6 Hz, 1H), 4.14 (d, J = 12.5 Hz, 1H), 3.97 (s, 3H), 3.76- 3.72 (m, 1H), 3.23 (t, J = 11.7 Hz, 1H), 2.98 (d, J = 10.8 Hz, 6H), 2.85 (t, J = 11.6 Hz, 1H), 2.12- |
| | | 2.07 (m, 2H), 1.51-1.37 (m, 2H). |
| 183 | 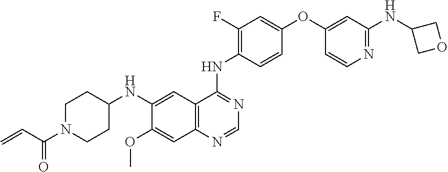 | LC/MS: 586.0 [M + H]⁺. ¹H NMR (400 MHz,) δ 9.13 (s, 1H), 8.62 (s, 1H), 8.21 (d, J = 7.3 Hz, 1H), 7.72 (t, J = 8.7 Hz, 1H), 7.54 (dd, J = 10.5, 2.5 Hz, 1H), 7.41 (s, 1H), 7.32-7.22 (m, 1H), 7.16 (s, 1H), 6.90- 6.78 (m, 1H), 6.69 (dd, J = 7.3, 2.5 Hz, 1H), 6.18 (d, J = 2.4 Hz, 1H), 6.13-6.06 (m, 1H), 6.01-5.90 (m, 1H), 5.66 (dd, J = 10.4, 2.4 Hz, 1H), 5.37-5.07 (m, 1H), 4.59 (t, J = 10.4 Hz, 1H), 4.48-4.40 (m, 1H), 4.36-4.25 (m, 2H), 4.16-4.07 (m, 1H), 4.00 (s, 3H), 3.78-3.71 (m, |
| | | 1H), 3.54-3.50 (m, 2H), 3.21-3.15 (m, 1H), |
| | | 2.87-2.75 (m, 1H), 2.07-1.94 (m, 2H), 1.54- |
| | | 1.36 (m, 2H). |
| 184 | 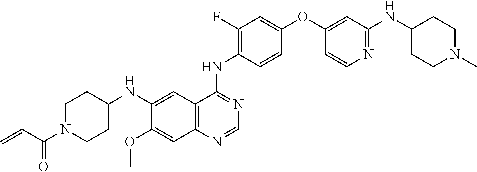 | LC/MS: 627.1 [M + H]⁺. ¹H NMR (400 MHz, DMSO) δ 9.18 (s, 1H), 8.23- 8.21 (m, 2H), 7.91 (d, J = 5.6 Hz, 1H), 7.58 (t, J = 8.8 Hz, 1H), 7.30-7.18 (m, 2H), 7.11-6.98 (m, 2H), 6.86 (dd, J = 16.8, 10.4 Hz, 1H), 6.58 (s, 1H), 6.22-6.08 (m, 2H), 5.99 (d, J = 2.0 Hz, 1H), 5.69 (dd, J = 10.4, 2.4 Hz, 1H), 5.33 (d, J = 8.4 Hz, 1H), 4.45 (d, J = 13.6 Hz, 1H), 4.13 (d, J = 12.4 Hz, 1H), 3.96 (s, 3H), 3.77-3.71 (m, 3H), 3.29-3.16 (m, 2H), 2.86-2.75 (d, J = 11.2 Hz, 3H), 2.30-2.22 (m, 3H), 2.14-2.03 |
| | | (m, 2H), 1.92-1.83 (m, 2H), 1.51-1.34 (m, |
| | | 4H). |
| 186 | 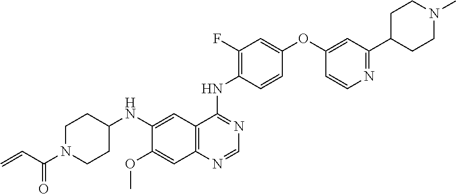 | LC/MS: 612.3 [M + H]⁺. ¹H NMR (400 MHz, DMSO) δ 9.21 (s, 1H), 8.44 (d, J = 5.6 Hz, 1H), 8.24 (s, 1H), 8.23-8.17 (m, 1H), 7.62 (t, J = 8.7 Hz, 1H), 7.33-7.25 (m, 2H), 7.13-7.05 (m, 2H), 6.94-6.79 (m, 3H), 6.12 (dd, J = 16.7, 2.3 Hz, 1H), 5.69 (dd, J = 10.5, 2.4 Hz, 1H), 5.34 (d, J = 8.4 Hz, 1H), 4.50- 4.42 (m, 1H), 4.17-4.09 (m, 1H), 3.97 (s, 3H), 3.77-3.74 (m, 1H), 3.25-3.22 (m, 1H), 2.99- 2.94 (m, 2H), 2.90-2.83 (m, 1H), 2.69-2.62 (m, 1H), 2.29 (s, 3H), 2.19-2.04 (m, 4H), 1.85- 1.71 (m, 4H), 1.50-1.39 (m, 2H). |
| 187 | 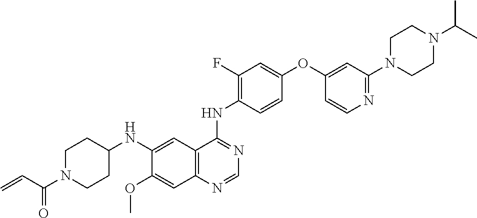 | LC/MS: 640.9 [M + H]⁺. ¹H NMR (400 MHz, DMSO) δ 9.17 (s, 1H), 8.22 (s, 1H), 8.15 (s, 1H), 8.06 (d, J = 5.7 Hz, 1H), 7.58-7.54 (m, 1H), 7.28-7.18 (m, 2H), 7.14- 7.04 (m, 2H), 6.91-6.82 (m, 1H), 6.41 (d, J = 1.9 Hz, 1H), 6.25 (dd, J = 5.7, 2.0 Hz, 1H), 6.12 (dd, J = 16.7, 2.5 Hz, 1H), 5.69 (dd, J = 10.5, 2.5 Hz, 1H), 5.33 (d, J = 8.6 Hz, 1H), 4.46 (d, J = 12.9 Hz, 1H), 4.14 (d, J = 13.4 Hz, 1H), 3.96 (s, 3H), 3.79-3.72 (m, 1H), 3.47-3.43 (m, 4H), 3.25-3.21 (m, 1H), 2.85 (t, J = 12.4 Hz, 1H), 2.77-2.68 (m, 1H), 2.57-2.52 (m, 3H), 2.12- 2.04 (m, 2H), 1.51-1.40 (m, 2H), 1.00 (d, J = |
| | | 6.5 Hz, 6H). |
| 188 | 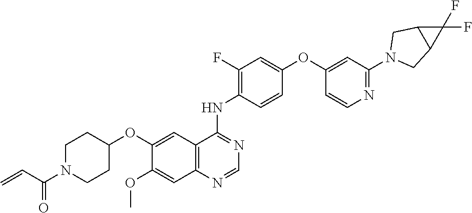 | LC/MS: 633.2 [M + H]⁺. ¹H NMR (400 MHz, DMSO) δ 9.46 (s, 1H), 8.37 (s, 1H), 8.04 (d, J = 5.8 Hz, 1H), 7.90 (s, 1H), 7.58 (t, J = 8.8 Hz, 1H), 7.25-7.21 (m, 2H), 7.04 (dd, J = 8.8, 1.8 Hz, 1H), 6.86 (dd, J = 16.8, 10.6 Hz, 1H), 6.25 (dd, J = 5.8, 2.2 Hz, 1H), 6.15-6.10 (m, 2H), 5.69 (dd, J = 10.6, 2.4 Hz, 1H), 4.85-4.72 (m, 1H), 3.94 (s, 3H), 3.74 (d, J = 11.0 Hz, 2H), 3.65 (d, J = 9.4 Hz, 2H), 3.53- 3.42 (m, 2H), 2.67 (d, J = 11.8 Hz, 2H), 2.09- 2.01 (m, 2H), 1.74-1.66 (m, 2H), 1.23-1.19 (m, 2H). |
| 189 | 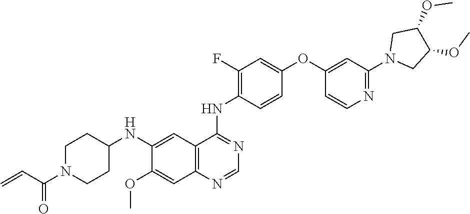 | LC/MS: 644.3 [M + H]⁺. ¹H NMR (400 MHz,) δ 9.20 (s, 1H), 8.23 (s, 1H), 8.02 (d, J = 5.7 Hz, 1H), 7.57 (t, J = 8.8 Hz, 1H), 7.26 (s, 1H), 7.20 (dd, J = 11.0, 2.7 Hz, 1H), 7.08 (s, 1H), 7.06-7.00 (m, 1H), 6.86 (dd, J = 16.7, 10.5 Hz, 1H), 6.21 (dd, J = 5.7, 2.1 Hz, 1H), 6.12 (dd, J = 16.7, 2.5 Hz, 1H), 6.03 (d, J = 2.1 Hz, 1H), 5.69 (dd, J = 10.4, 2.5 Hz, 1H), 5.35 (d, J = 8.5 Hz, 1H), 4.53-4.39 (m, 1H), 4.19-4.09 (m, 1H), 4.02 (t, J = 4.0 Hz, 2H), 3.97 (s, 3H), 3.81-3.69 (m, 1H), 3.55-3.47 (m, 2H), 3.38-3.34 (m, 2H), 3.33 (s, 6H), 3.28- 3.17 (m, 1H), 2.92-2.80 (m, 1H), 2.12-2.02 (m, 2H), 1.50-1.38 (m, 2H). |
| 190 | 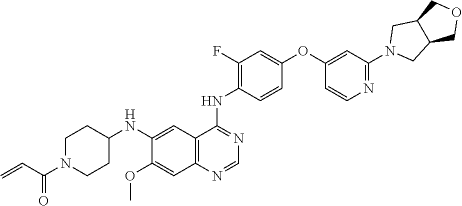 | LC/MS: 626.2 [M + H]+. 1H NMR (400 MHz, DMSO) δ 10.92 (s, 1H), 8.72 (s, 1H), 8.08 (d, J = 6.4 Hz, 1H), 7.72 (t, J = 8.8 Hz, 1H), 7.54-7.47 (m, 2H), 7.29-7.22 (m, 2H), 6.87 (dd, J = 16.8, 10.5 Hz, 1H), 6.60- 6.54 (m, 1H), 6.39 (s, 1H), 6.13 (dd, J = 16.7, 2.4 Hz, 1H), 6.05 (brs, 1H), 5.70 (dd, J = 10.4, 2.4 Hz, 1H), 4.48 (d, J = 12.4 Hz, 1H), 4.16 (d, J = 12.0 Hz, 1H), 4.05 (s, 3H), 3.89-3.75 (m, 3H), 3.73-3.67 (m, 2H), 3.63 (dd, J = 8.8, 3.2 Hz, 2H), 3.43 (d, J = 10.8 Hz, 2H), 3.22 (t, J = 12.4 Hz, 1H), 3.18-3.11 (m, 2H), 2.84 (t, J = 12.0 Hz, 1H), 2.04 (d, J = 11.2 Hz, 2H), 1.58- 1.42 (m, 2H). |
| 192 | 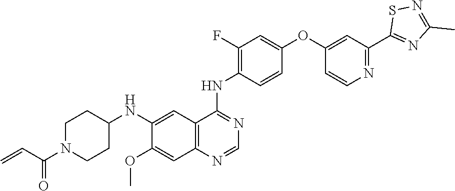 | LC/MS: 613.2 [M + H]⁺. ¹H NMR (400 MHz, DMSO) δ 9.34 (s, 1H), 8.67 (d, J = 5.7 Hz, 1H), 8.29 (s, 1H), 7.70 (t, J = 8.7 Hz, 1H), 7.54 (d, J = 2.5 Hz, 1H), 7.46 (dd, J = 10.7, 2.6 Hz, 1H), 7.35 (dd, J = 5.7, 2.5 Hz, 1H), 7.28 (s, 1H), 7.23 (dd, J = 8.6, 2.0 Hz, 1H), 7.10 (s, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.41 (d, J = 8.4 Hz, 1H), 4.47 (d, J = 12.4 Hz, 1H), 4.14 (d, J = 11.4 Hz, 1H), 3.98 (s, 3H), 3.76 (d, J = 8.4 Hz, 1H), 3.24 (t, J = 12.4 Hz, 1H), 2.86 (t, J = 11.9 Hz, 1H), 2.65 (s, 3H), |
| | | 2.12-2.05 (m, 2H), 1.52-1.40 (m, 2H). |
| 194 | 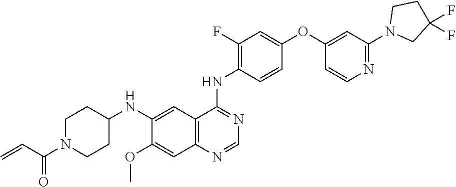 | LC/MS: 620.3 [M + H]⁺. ¹H NMR (400 MHz, MeOD) δ 8.23 (s, 1H), 8.00 (d, J = 5.9 Hz, 1H), 7.61 (t, J = 8.7 Hz, 1H), 7.28 (s, 1H), 7.11-7.02 (m, 3H), 6.85-6.78 (m, 1H), 6.41 (dd, J = 5.9, 2.1 Hz, 1H), 6.21 (dd, J = 16.8, 2.0 Hz, 1H), 6.16 (d, J = 2.0 Hz, 1H), 5.75 (dd, J = 10.7, 2.0 Hz, 1H), 4.67-4.53 (m, 1H), 4.19 (d, J = 13.0 Hz, 1H), 4.04 (s, 3H), 3.88-3.75 (m, 3H), 3.63 (t, J = 7.3 Hz, 2H), 3.40-3.32 (m, 1H), 3.05-2.95 (m, 1H), 2.56-2.47 (m, 2H), 2.27-2.17(m, 2H), 1.56-1.47 (m, 2H). |
| 195 | 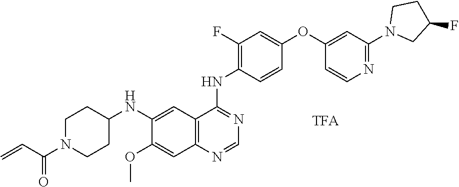 | LC/MS: 602.1 [M + H]⁺. 1H NMR (400 MHz, CD₃OD) δ 8.56 (s, 1H), 7.96 (d, J = 7.3 Hz, 1H), 7.74 (t, J = 8.6 Hz, 1H), 7.47 (s, 1H), 7.35 (dd, J = 10.3, 2.6 Hz, 1H), 7.26-7.22 (m, 1H), 7.19 (s, 1H), 6.82 (dd, J = 16.8, 10.7 Hz, 1H), 6.71 (dd, J = 7.3, 2.4 Hz, 1H), 6.46 (d, J = 2.3 Hz, 1H), 6.22 (dd, J = 16.8, 2.0 Hz, 1H), 5.76 (dd, J = 10.6, 2.0 Hz, 1H), 5.57-5.40 (m, 1H), 4.60 (d, J = 13.5 Hz, 1H), 4.21 (d, J = 14.2 Hz, 1H), 4.12 (s, 3H), 3.87- 3.66 (m, 5H), 3.41-3.33 (m, 1H), 2.99 (t, J = 11.9 Hz, 1H), 2.54-2.29 (m, 2H), 2.21 (d, J = |
| | | 12.3 Hz, 2H), 1.63-1.48 (m, 2H). |
| 196 | 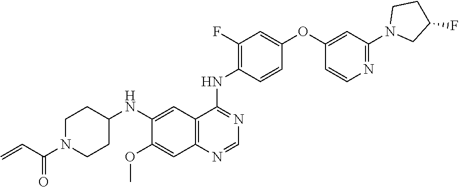 | LC/MS: 602.1 [M + H]⁺. ¹H NMR (400 MHz, DMSO-d6) δ 9.45 (brs, 1H), 8.30 (s, 1H), 8.05 (d, J = 5.7 Hz, 1H), 7.58 (t, J = 8.8 Hz, 1H), 7.30 (s, 1H), 7.23 (dd, J = 10.9, 2.5 Hz, 1H), 7.09 (s, 1H), 7.05 (dd, J = 8.7, 2.0 Hz, 1H), 6.86 (dd, J = 16.7, 10.5 Hz, 1H), 6.24 (dd, J = 5.7, 2.0 Hz, 1H), 6.12 (dd, J = 13.0, 3.7 Hz, 1H), 6.09 (d, J = 2.1 Hz, 1H), 5.69 (dd, J = 10.4, 2.4 Hz, 1H), 5.51-5.34 (m, 2H), 4.46 (d, J = 12.1 Hz, 1H), 4.14 (d, J = 12.4 Hz, 1H), 3.97 (s, 3H), 3.78-3.50 (m, 4H), 3.37-3.41 (m, 1H), 3.26-3.18 (m, 1H), 2.85 |
| | | (t, J = 12.0 Hz, 1H), 2.04-2.26 (m, 4H), 1.49- |
| | | 1.38 (m, 2H). |
| 197 | 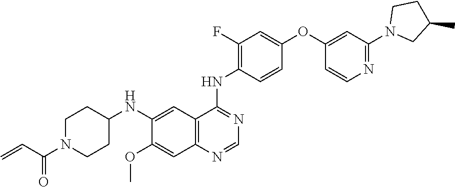 | LC/MS: 598.3 [M + H]⁺. ¹H NMR (400 MHz, DMSO-d6) δ 9.23 (brs, 1H), 8.24 (s, 1H), 8.01 (d, J = 5.7 Hz, 1H), 7.57 (t, J = 8.8 Hz, 1H), 7.26 (s, 1H), 7.20 (dd, J = 11.0, 2.6 Hz, 1H), 7.08 (s, 1H), 7.03 (dd, J = 8.6, 1.9 Hz, 1H), 6.86 (dd, J = 16.7, 10.5 Hz, 1H), 6.19 (dd, J = 5.7, 2.1 Hz, 1H), 6.12 (dd, J = 16.7, 2.5 Hz, 1H), 5.96 (d, J = 2.1 Hz, 1H), 5.69 (dd, J = 10.4, 2.5 Hz, 1H), 5.36 (d, J = 8.7 Hz, 1H), 4.46 (d, J = 13.9 Hz, 1H), 4.14 (d, J = 12.2 Hz, 1H), 3.97 (s, 3H), 3.79-3.72 (m, 1H), 3.58-3.51 (m, 1H), 3.49-3.41 (m, 1H), 3.30-3.19 (m, 2H), |
| | | 2.90-2.82 (m, 2H), 2.41-2.28 (m, 1H), 2.10- |
| | | 2.03 (m, 3H), 1.60-1.52 (m, 1H), 1.49-1.39 |
| | | (m, 2H), 1.06 (d, J = 6.6 Hz, 3H). |
| 198 | 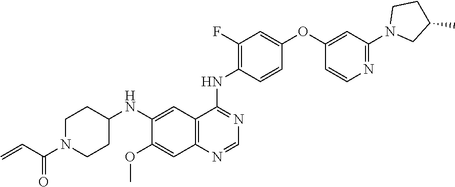 | LC/MS: 598.3 [M + H]⁺. ¹H NMR (400 MHz, DMSO) δ 10.87 (s, 1H), 8.71 (s, 1H), 8.05 (d, J = 6.9 Hz, 1H), 7.71 (t, J = 8.7 Hz, 1H), 7.55-7.45 (m, 2H), 7.29-7.21 (m, 2H), 6.87 (dd, J = 16.7, 10.5 Hz, 1H), 6.53 (d, J = 6.7 Hz, 1H), 6.33 (s, 1H), 6.13 (dd, J = 16.7, 2.4 Hz, 1H), 6.04 (d, J = 6.8 Hz, 1H), 5.70 (dd, J = 10.4, 2.4 Hz, 1H), 4.48 (d, J = 12.8 Hz, 1H), 4.16 (d, J = 12.9 Hz, 1H), 4.04 (s, 3H), 3.79- 3.75 (m, 1H), 3.67-3.54 (m, 2H), 3.46-3.39 (m, 1H), 3.21 (t, J = 12.0 Hz, 1H), 3.03-2.97 (m, 1H), 2.84 (t, J = 12.2 Hz, 1H), 2.40 (dd, J = |
| | | 14.8, 7.2 Hz, 1H), 2.19-2.01 (m, 3H), 1.69- |
| | | 1.60 (m, 1H), 1.57-1.43 (m, 2H), 1.08 (d, J = |
| | | 6.6 Hz, 3H). |
| 199 | 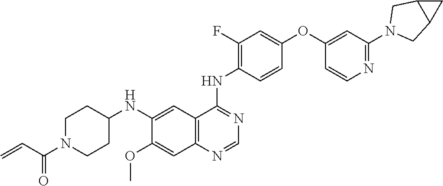 | LC/MS: 596.3 [M + H]⁺. ¹H NMR (400 MHz, DMSO) δ 9.05 (s, 1H), 8.06 (s, 1H), 7.89-7.78 (m, 1H), 7.46-7.34 (m, 1H), 7.17-6.81 (m, 4H), 6.79-6.64 (m, 1H), 6.10- 5.80 (m, 3H), 5.58-5.46 (m, 1H), 5.25-5.11 (m, 1H), 4.39-4.19 (m, 1H), 4.04-3.90 (m, 1H), 3.80 (s, 3H), 3.66-3.51 (m, 1H), 3.44- 3.38 (m, 2H), 3.09-3.01 (m, 2H), 2.79-2.57 (m, 2H), 1.99-1.81 (m, 2H), 1.56-1.41 (m, 2H), 1.34-1.19 (m, 2H), 1.10-1.01 (m, 1H), 0.61-0.51 (m, 1H). |
| 200 | 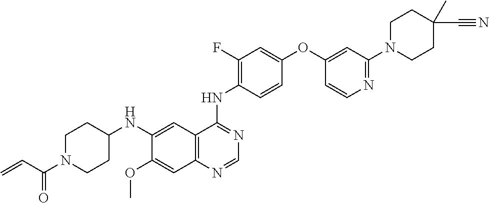 | LC/MS: 636.8 [M + H]⁺. ¹H NMR (400 MHz, DMSO) δ 9.23 (s, 1H), 8.24 (s, 1H), 8.08 (d, J = 5.7 Hz, 1H), 7.58 (t, J = 8.8 Hz, 1H), 7.27-7.19 (m, 2H), 7.09-7.00 (m, 2H), 6.87 (dd, J = 16.7, 10.5 Hz, 1H), 6.52 (d, J = 1.9 Hz, 1H), 6.27 (dd, J = 5.7, 2.0 Hz, 1H), 6.12 (dd, J = 16.7, 2.5 Hz, 1H), 5.69 (dd, J = 10.4, 2.5 Hz, 1H), 5.36 (d, J = 8.6 Hz, 1H), 4.46 (d, J = 11.7 Hz, 1H), 4.26 (d, J = 13.8 Hz, 2H), 4.14 (d, J = 12.4 Hz, 1H), 3.97 (s, 3H), 3.80- 3.70 (m, 1H), 3.26-3.19 (m, 1H), 3.05-2.92 (m, 2H), 2.89-2.84 (m, 1H), 2.13-2.06 (m, 2H), 1.96-1.90 (m, 2H), 1.56-1.41 (m, 4H), |
| | | 1.37 (s, 3H). |
| 201 | 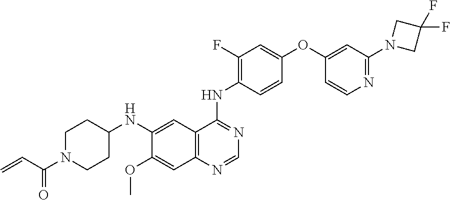 | LC/MS: 606.0 [M + H]⁺. ¹H NMR (400 MHz, DMSO) δ 10.85 (s, 1H), 8.72 (s, 1H), 8.15 (d, J = 6.0 Hz, 1H), 7.66 (t, J = 8.7 Hz, 1H), 7.46 (s, 1H), 7.41 (dd, J = 10.8, 2.6 Hz, 1H), 7.22-7.15 (m, 2H), 6.87 (dd, J = 16.7, 10.5 Hz, 1H), 6.51 (dd, J = 6.0, 2.2 Hz, 1H), 6.25 (d, J = 2.1 Hz, 1H), 6.17-5.98 (m, 2H), 5.70 (dd, J = 10.4, 2.4 Hz, 1H), 4.54-4.36 (m, 5H), 4.16 (d, J = 12.5 Hz, 1H), 4.05 (s, 3H), 3.83-3.70 (m, 1H), 3.21 (t, J = 12.4 Hz, 1H), 2.83 (t, J = 12.1 Hz, 1H), 2.10-1.97 (m, 2H), 1.58-1.41 (m, 2H). |
| 202 | 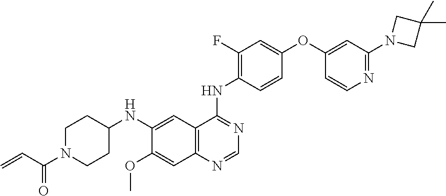 | LC/MS: 597.8 [M + H]⁺. ¹H NMR (400 MHz, DMSO) δ 9.19 (s, 1H), 8.22 (s, 1H), 8.00 (d, J = 5.7 Hz, 1H), 7.58 (t, J = 8.8 Hz, 1H), 7.26 (s, 1H), 7.21 (dd, J = 10.9, 2.6 Hz, 1H), 7.08 (s, 1H), 7.03 (dd, J = 8.6, 1.8 Hz, 1H), 6.86 (dd, J = 16.7, 10.5 Hz, 1H), 6.26 (dd, J = 5.7, 2.2 Hz, 1H), 6.12 (dd, J = 16.7, 2.5 Hz, 1H), 5.87 (d, J = 2.1 Hz, 1H), 5.69 (dd, J = 10.4, 2.5 Hz, 1H), 5.34 (d, J = 8.5 Hz, 1H), 4.46 (d, J = 13.2 Hz, 1H), 4.14 (d, J = 11.5 Hz, 1H), 3.96 (s, 3H), 3.75 (d, J = 8.8 Hz, 1H), 3.60 (s, 4H), 3.23- 3.16 (m, 1H), 2.92-2.83 (m, 1H), 2.11-2.03 (m, 2H), 1.52-1.41 (m, 2H), 1.27 (s, 6H). |
| 203 | 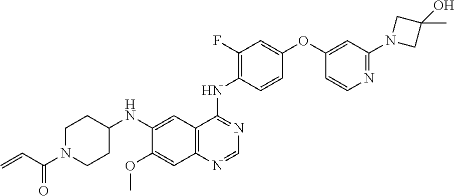 [SMILES: C=CC(=O)N1CCC(Nc2cc3c(Nc4ccc(Oc5ccnc(N6CC(C)(O)C6)c5)cc4F)ncnc3cc2OC)CC1] | LC/MS: 599.8 [M + H]⁺. ¹H NMR (400 MHz, DMSO-d6) δ 9.23 (s, 1H), 8.24 (s, 1H), 8.01 (d, J = 5.8 Hz, 1H), 7.58 (t, J = 8.8 Hz, 1H), 7.26 (s, 1H), 7.21 (dd, J = 10.9, 2.7 Hz, 1H), 7.08 (s, 1H), 7.03 (dd, J = 8.7, 1.8 Hz, 1H), 6.86 (dd, J = 16.7, 10.5 Hz, 1H), 6.27 (dd, J = 5.7, 2.2 Hz, 1H), 6.12 (dd, J = 16.7, 2.5 Hz, 1H), 5.93 (d, J = 2.1 Hz, 1H), 5.69 (dd, J = 10.4, 2.5 Hz, 1H), 5.56 (s, 1H), 5.36 (d, J = 8.6 Hz, 1H), 4.46 (d, J = 11.8 Hz, 1H), 4.14 (d, J = 13.5 Hz, 1H), 3.97 (s, 3H), 3.81-3.72 (m, 5H), 3.23 (t, J = 13.0 Hz, 1H), 2.85 (t, J = 11.6 Hz, 1H), 2.12-2.05 (m, 2H), 1.47-1.39 (m, 5H). |
| 204 | 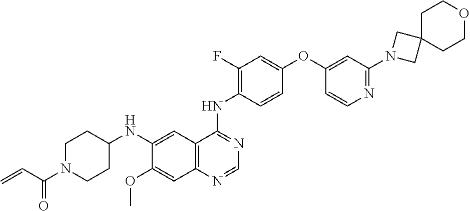 [SMILES: C=CC(=O)N1CCC(Nc2cc3c(Nc4ccc(Oc5ccnc(N6CC7(CCOCC7)C6)c5)cc4F)ncnc3cc2OC)CC1] | LC/MS: 640.3 [M + H]⁺. 1H NMR (400 MHz, DMSO-d6) δ 9.19 (brs, 1H), 8.23 (s, 1H), 8.01 (d, J = 5.7 Hz, 1H), 7.58 (t, J = 8.8 Hz, 1H), 7.26 (s, 1H), 7.22 (dd, J = 10.8, 2.4 Hz, 1H), 7.08 (s, 1H), 7.04 (d, J = 8.3 Hz, 1H), 6.87 (dd, J = 16.6, 10.5 Hz, 1H), 6.32- 6.24 (m, 1H), 6.12 (dd, J = 16.7, 2.3 Hz, 1H), 5.88 (d, J = 1.7 Hz, 1H), 5.69 (dd, J = 10.5, 2.3 Hz, 1H), 5.34 (d, J = 8.5 Hz, 1H), 4.47 (d, J = 11.6 Hz, 1H), 4.14 (d, J = 11.7 Hz, 1H), 3.96 (s, 3H), 3.77-3.72 (m, 1H), 3.72-3.64 (m, 4H), 3.55-3.50 (m, 4H), 3.25-3.20 (m, 1H), 2.85 (t, J = 11.9 Hz, 1H), 2.10-2.00 (m, 2H), 1.75- 1.60 (m, 4H), 1.50-1.40 (m, 2H). |
| 205 | 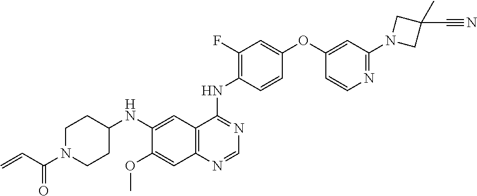 [SMILES: C=CC(=O)N1CCC(Nc2cc3c(Nc4ccc(Oc5ccnc(N6CC(C)(C#N)C6)c5)cc4F)ncnc3cc2OC)CC1] | LC/MS: 609.3 [M + H]⁺. ¹H NMR (400 MHz, DMSO) δ 9.20 (s, 1H), 8.24 (d, J = 7.6 Hz, 1H), 8.07 (d, J = 5.6 Hz, 1H), 7.59 (t, J = 8.8 Hz, 1H), 7.29-7.18 (m, 2H), 7.10-7.01 (m, 2H), 6.87 (dd, J = 16.8, 10.4 Hz, 1H), 6.37 (dd, J = 5.6, 2.0 Hz, 1H), 6.12 (dd, J = 16.8, 2.4 Hz, 1H), 6.05 (d, J = 2.0 Hz, 1H), 5.69 (dd, J = 10.4, 2.4 Hz, 1H), 5.34 (d, J = 8.4 Hz, 1H), 4.46 (d, J = 12.0 Hz, 1H), 4.23 (d, J = 8.2 Hz, 2H), 4.14 (d, J = 13.2 Hz, 1H), 3.96 (s, 2H), 3.90 (d, J = 8.0 Hz, 2H), 3.75 (d, J = 8.4 Hz, 1H), 3.26-3.19 (m, 1H), 2.85 (t, J = 11.6 |
| | | Hz, 1H), 2.16-2.03 (m, 2H), 1.65 (s, 3H), 1.49- |
| | | 1.38 (m, 2H), 1.27-1.22 (m, 1H). |
| 206 | 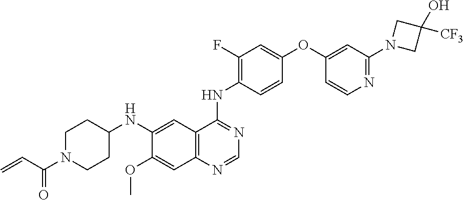 [SMILES: C=CC(=O)N1CCC(Nc2cc3c(Nc4ccc(Oc5ccnc(N6CC(O)(C(F)(F)F)C6)c5)cc4F)ncnc3cc2OC)CC1] | LC/MS: 654.0 [M + H]⁺. ¹H NMR (400 MHz,) δ 9.19 (s, 1H), 8.23 (s, 2H), 8.06 (d, J = 5.8 Hz, 1H), 7.59 (t, J = 8.8 Hz, 1H), 7.24 (dd, J = 11.9, 3.5 Hz, 2H), 7.08 (s, 1H), 7.05 (dd, J = 8.7, 2.0 Hz, 1H), 6.87 (dd, J = 16.7, 10.5 Hz, 1H), 6.34 (dd, J = 5.7, 2.1 Hz, 1H), 6.12 (dd, J = 16.8, 2.3 Hz, 2H), 5.69 (dd, J = 10.4, 2.4 Hz, 1H), 5.35 (d, J = 8.5 Hz, 1H), 4.46 (d, J = 12.3 Hz, 1H), 4.19 (d, J = 9.8 Hz, 2H), 4.14 (d, J = 12.6 Hz, 1H), 3.96 (s, 3H), 3.93 (d, J = 9.7 Hz, 2H), 3.78-3.72 (m, 1H), 3.21 (d, J = 12.2 Hz, 2H), 2.85 (t, J = 11.8 Hz, 1H), 2.07 (d, J = 3.3 Hz, 2H), 1.49-1.38 (m, 2H). |
| 207 | 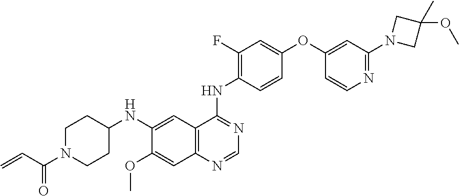 [SMILES: C=CC(=O)N1CCC(Nc2cc3c(Nc4ccc(Oc5ccnc(N6CC(C)(OC)C6)c5)cc4F)ncnc3cc2OC)CC1] | LC/MS: 614.0 [M + H]⁺. ¹H NMR (400 MHz, DMSO) δ 9.20 (s, 1H), 8.23 (s, 1H), 8.02 (d, J = 5.7 Hz, 1H), 7.58 (t, J = 8.7 Hz, 1H), 7.27-7.20 (m, 2H), 7.09-7.01 (m, 2H), 6.86 (dd, J = 16.7, 10.5 Hz, 1H), 6.29 (dd, J = 5.7, 1.9 Hz, 1H), 6.12 (dd, J = 16.7, 2.3 Hz, 1H), 5.95 (d, J = 1.7 Hz, 1H), 5.69 (dd, J = 10.4, 2.2 Hz, 1H), 5.34 (d, J = 8.5 Hz, 1H), 4.46 (d, J = 12.1 Hz, 1H), 4.14 (d, J = 12.9 Hz, 1H), 3.96 (s, 3H), 3.84 (d, J = 8.5 Hz, 2H), 3.72 (d, J = 8.5 Hz, 3H), 3.16-3.25 (m, 4H), 2.91-2.82 (m, 1H), 2.10-2.03 (m, 2H), 1.40-1.48 (m, 5H). |
| 208 | 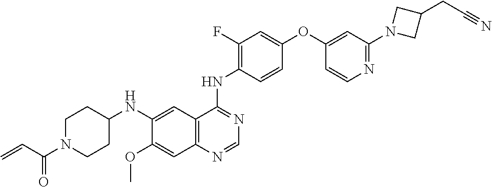 [SMILES: C=CC(=O)N1CCC(Nc2cc3c(Nc4ccc(Oc5ccnc(N6CC(CC#N)C6)c5)cc4F)ncnc3cc2OC)CC1] | LC/MS: 609.3 [M + H]⁺. ¹H NMR (400 MHz,) δ 9.18 (s, 1H), 8.28 (s, 1H), 8.22 (s, 1H), 8.03 (d, J = 5.8 Hz, 1H), 7.58 (t, J = 8.8 Hz, 1H), 7.27-7.19 (m, 2H), 7.09- 7.01 (m, 2H), 6.86 (dd, J = 16.7, 10.5 Hz, 1H), 6.29 (dd, J = 5.8, 2.2 Hz, 1H), 6.11 (dd, J = 16.6, 2.5 Hz, 1H), 5.95 (d, J = 2.1 Hz, 1H), 5.68 (dd, J = 10.4, 2.5 Hz, 1H), 5.33 (d, J = 8.5 Hz, 1H), 4.46 (d, J = 12.9 Hz, 1H), 4.13 (d, J = 12.1 Hz, 1H), 4.05 (t, J = 8.1 Hz, 2H), 3.96 (s, 3H), 3.78- 3.72 (m, 1H), 3.65 (dd, J = 8.4, 5.5 Hz, 2H), 3.25-3.21 (m, 1H), 3.04-2.98 (m, 1H), 2.91- |
| | | 2.83 (m, 3H), 2.11-2.03 (m, 2H), 1.48-1.41 |
| | | (m, 2H). |
| 209 | 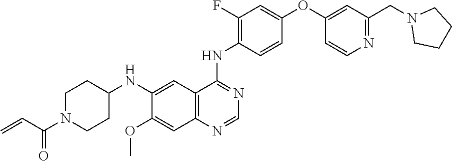 [SMILES: C=CC(=O)N1CCC(Nc2cc3c(Nc4ccc(Oc5ccnc(CN6CCCC6)c5)cc4F)ncnc3cc2OC)CC1] | LC/MS: 598.3 [M + H]⁺. ¹H NMR (400 MHz, DMSO) δ 9.23 (s, 1H), 8.49 (d, J = 4.6 Hz, 1H), 8.24 (s, 1H), 7.64 (t, J = 8.8 Hz, 1H), 7.32 (dd, J = 10.9, 2.4 Hz, 1H), 7.27 (s, 1H), 7.13-7.07 (m, 3H), 6.99 (s, 1H), 6.87 (dd, J = 6.2 Hz, 1H), 6.14-6.09 (m, 1H), 5.69 (dd, J = 10.4, 2.4 Hz, 1H), 5.36 (d, J = 8.9 Hz, 1H), 4.49-4.44 (m, 1H), 4.12 (d, 1H), 3.97 (s, 3H), 3.82-3.75 (m, 1H), 3.32-3.10 (m, 4H), 2.91-2.83 (m, 4H), 2.10-2.04 (m, 2H), 1.83- 1.79 (m, 3H), 1.37-1.49 (m, 3H). |
| 211 | 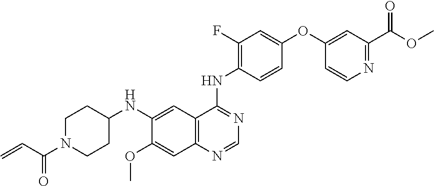 [SMILES: C=CC(=O)N1CCC(Nc2cc3c(Nc4ccc(Oc5ccnc(C(=O)OC)c5)cc4F)ncnc3cc2OC)CC1] | LC/MS: 572.8 [M + H]⁺. ¹H NMR (400 MHz, DMSO) δ 9.23 (s, 1H), 8.66 (d, J = 5.6 Hz, 1H), 8.25 (s, 1H), 7.67 (t, J = 8.8 Hz, 1H), 7.52 (d, J = 2.4 Hz, 1H), 7.39 (dd, J = 10.8, 2.7 Hz, 1H), 7.31 (dd, J = 5.6, 2.6 Hz, 1H), 7.26 (s, 1H), 7.17 (dd, J = 8.6, 1.8 Hz, 1H), 7.09 (s, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.35 (d, J = 8.5 Hz, 1H), 4.46 (d, J = 12.8 Hz, 1H), 4.14 (d, J = 12.6 Hz, 1H), 3.97 (s, 3H), 3.86 (d, J = 8.0 Hz, 3H), 3.80-3.71 (m, 1H), 3.28-3.17 (m, 1H), 2.86 (t, J = 11.9 Hz, |
| | | 1H), 2.13-2.02 (m, 2H), 1.52-1.37 (m, 2H). |
| 212 | 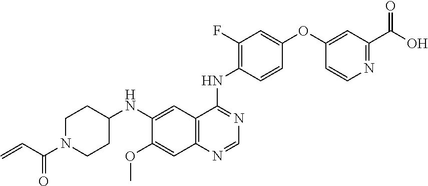 [SMILES: C=CC(=O)N1CCC(Nc2cc3c(Nc4ccc(Oc5ccnc(C(=O)O)c5)cc4F)ncnc3cc2OC)CC1] | LC/MS: 558.9 [M + H]⁺. ¹H NMR (400 MHz, DMSO) δ 14.52 (brs, 1H), 10.81 (s, 1H), 8.75 (s, 1H), 8.68 (d, J = 5.6 Hz, 1H), 7.73 (t, J = 8.7 Hz, 1H), 7.57-7.48 (m, 2H), 7.44 (s, 1H), 7.35 (dd, J = 5.6, 2.6 Hz, 1H), 7.28 (dd, J = 8.7, 1.9 Hz, 1H), 7.15 (s, 1H), 6.87 (dd, J = 16.7, 10.5 Hz, 1H), 6.17-6.01 (m, 2H), 5.70 (dd, J = 10.4, 2.4 Hz, 1H), 4.49 (d, J = 13.1 Hz, 1H), 4.17 (d, J = 12.0 Hz, 1H), 4.05 (s, 3H), 3.25-3.19 (m, 1H), 2.88-2.81 (m, 1H), 2.09- 2.00 (m, 3H), 1.54-1.42 (m, 2H). |
| 213 | 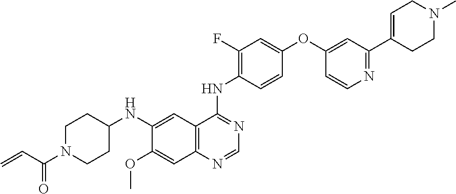 [SMILES: C=CC(=O)N1CCC(Nc2cc3c(Nc4ccc(Oc5ccnc(C6=CCN(C)CC6)c5)cc4F)ncnc3cc2OC)CC1] | LC/MS: 610.3 [M + H]⁺. ¹H NMR (400 MHz, DMSO) δ 9.22 (s, 1H), 8.47 (d, J = 5.6 Hz, 1H), 8.23 (s, 1H), 8.19 (s, 1H), 7.61 (t, J = 8.8 Hz, 1H), 7.33-7.25 (m, 2H), 7.14 (d, J = 2.3 Hz, 1H), 7.12-7.06 (m, 2H), 6.94-6.81 (m, 2H), 6.73-6.65 (m, 1H), 6.16- 6.06 (m, 1H), 5.69 (dd, J = 10.4, 2.5 Hz, 1H), 5.35 (d, J = 8.5 Hz, 1H), 4.52-4.42 (m, 1H), 4.18-4.10 (m, 1H), 3.97 (s, 3H), 3.79-3.72 (m, 1H), 3.26-3.19 (m, 1H), 3.13-3.07 (m, 2H), 2.91-2.81 (m, 1H), 2.67-2.58 (m, 2H), 2.57- 2.53 (m, 2H), 2.31 (s, 3H), 2.13-2.01 (m, 2H), 1.51-1.38 (m, 2H). |
| 214 | 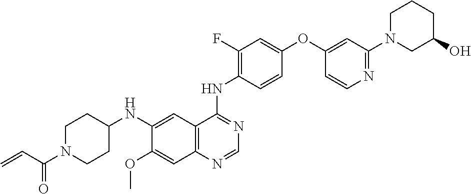 [SMILES: C=CC(=O)N1CCC(Nc2cc3c(Nc4ccc(Oc5ccnc(N6CCC[C@@H](O)C6)c5)cc4F)ncnc3cc2OC)CC1] | LC/MS: 614.3 [M + H]⁺. ¹H NMR (400 MHz, DMSO) δ 10.91 (s, 1H), 8.72 (s, 1H), 8.05 (d, J = 6.8 Hz, 1H), 7.70 (t, J = 8.8 Hz, 1H), 7.49-7.45 (m, 2H), 7.26-7.21 (m, 2H), 6.87 (dd, J = 16.8, 10.4 Hz, 1H), 6.71 (s, 1H), 6.44 (d, J = 6.4 Hz, 1H), 6.13 (dd, J = 16.8, 2.4 Hz, 1H), 6.05 (brs, 1H), 5.70 (dd, J = 10.4, 2.4 Hz, 1H), 4.48 (d, J = 12.0 Hz, 1H), 4.16 (d, J = 13.6 Hz, 1H), 4.05 (s, 3H), 3.89- 3.62 (m, 5H), 3.41-3.34 (m, 1H), 3.25-3.17 (m, 2H), 2.84 (t, J = 11.6 Hz, 1H), 2.09-2.01 (m, 2H), 1.89-1.77 (m, 2H), 1.54-1.45 (m, 4H). |
| 215 | 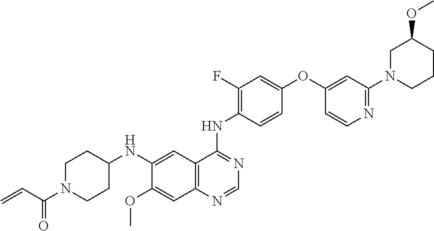 [SMILES: C=CC(=O)N1CCC(Nc2cc3c(Nc4ccc(Oc5ccnc(N6CCC[C@H](OC)C6)c5)cc4F)ncnc3cc2OC)CC1] | LCMS: 628.1 [M + H]⁺. 1H NMR (400 MHz, DMSO) δ = 10.37 (brs, 1H), 8.38 (s, 1H), 8.04 (d, J = 5.6 Hz, 1H), 7.67 (s, 1H), 7.57 (t, J = 8.8 Hz, 1H), 7.25-7.16 (m, 2H), 7.04 (dd, J = 8.4, 2.0 Hz, 1H), 6.86 (dd, J = 16.8, 10.4 Hz, 1H), 6.46 (d, J = 2.0 Hz, 1H), 6.22 (dd, J = 5.6, 2.0 Hz, 1H), 6.11 (dd, J = 16.8, 2.4 Hz, 1H), 5.68 (dd, J = 10.4, 2.4 Hz, 1H), 5.54 (d, J = 8.0 Hz, 1H), 4.46 (d, J = 11.6 Hz, 1H), 4.11 (d, J = 12.0 Hz, 1H), 4.06-3.87 (m, 5H), 3.76-3.66 (m, 1H), 3.30-3.08 (m, 7H), 2.89 (t, J = 12.0 Hz, 1H), 2.12-2.02 (m, 2H), 1.98-1.89 (m, 1H), 1.74-1.64 (m, 1H), 1.51- 1.35 (m, 4H). |
| 216 | 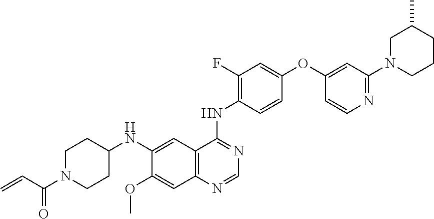 [SMILES: C=CC(=O)N1CCC(Nc2cc3c(Nc4ccc(Oc5ccnc(N6CCC[C@@H](C)C6)c5)cc4F)ncnc3cc2OC)CC1] | LCMS: 612.1 [M + H]⁺. 1H NMR (400 MHz, DMSO) δ 9.18 (s, 1H), 8.22 (s, 1H), 8.04 (d, J = 5.6 Hz, 1H), 7.57 (t, J = 8.8 Hz, 1H), 7.26 (s, 1H), 7.20 (dd, J = 10.8, 2.4 Hz, 1H), 7.08 (s, 1H), 7.02 (dd, J = 8.8, 2.0 Hz, 1H), 6.86 (dd, J = 16.8, 10.4 Hz, 1H), 6.42 (d, J = 2.0 Hz, 1H), 6.18 (dd, J = 5.6, 2.0 Hz, 1H), 6.12 (dd, J = 16.8, 2.4 Hz, 1H), 5.69 (dd, J = 10.4, 2.4 Hz, 1H), 5.33 (d, J = 8.8 Hz, 1H), 4.46 (d, J = 12.4 Hz, 1H), 4.20-4.08 (m, 3H), 3.96 (s, 3H), 3.81- 3.71 (m, 1H), 3.23 (t, J = 12.0 Hz, 1H), 2.85 (t, J = 11.2 Hz, 1H), 2.76 (td, J = 12.0, 2.8 Hz, 1H), 2.44 (t, J = 12.0 Hz, 1H), 2.14-2.03 (m, 2H), 1.80-1.73 (m, 1H), 1.68-1.60 (m, 1H), 1.61- |
| | | 1.51 (m, 1H), 1.51-1.39 (m, 3H), 1.18-1.05 |
| | | (m, 1H), 0.89 (d, J = 6.4 Hz, 3H). |
| 217 | 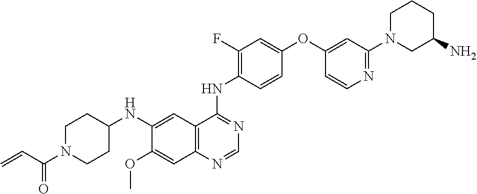 [SMILES: C=CC(=O)N1CCC(Nc2cc3c(Nc4ccc(Oc5ccnc(N6CCC[C@@H](N)C6)c5)cc4F)ncnc3cc2OC)CC1] | LC/MS: 613.3 [M + H]⁺. ¹H NMR (400 MHz, DMSO) δ 9.26 (s, 1H), 8.22 (s, 1H), 8.06 (d, J = 5.7 Hz, 1H), 7.58 (t, J = 8.8 Hz, 1H), 7.30 (s, 1H), 7.23-7.16 (m, 1H), 7.07 (s, 1H), 7.02 (dd, J = 8.7, 1.9 Hz, 1H), 6.86 (dd, J = 16.7, 10.5 Hz, 1H), 6.46 (d, J = 1.8 Hz, 1H), 6.25 (dd, J = 5.7, 1.9 Hz, 1H), 6.12 (dd, J = 16.7, 2.4 Hz, 1H), 5.69 (dd, J = 10.4, 2.4 Hz, 1H), 5.34 (d, J = 8.6 Hz, 1H), 4.46 (d, J = 13.0 Hz, 1H), 4.25 (d, J = 10.5 Hz, 1H), 4.13 (d, J = 12.8 Hz, 1H), 3.95-3.88 (m, 1H), 3.80-3.72 (m, 1H), 3.42-3.33 (m, 4H), 3.25-3.16 (m, 2H), 2.95-2.83 (m, 4H), 2.13-2.04 (m, 2H), 1.98- |
| | | 1.90 (m, 1H), 1.76-1.70 (m, 1H), 1.50-1.40 |
| | | (m, 3H), 1.23 (d, J = 3.4 Hz, 1H). |
| 218 | 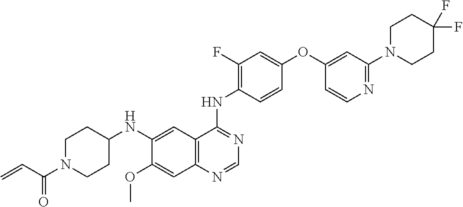 [SMILES: C=CC(=O)N1CCC(Nc2cc3c(Nc4ccc(Oc5ccnc(N6CCC(F)(F)CC6)c5)cc4F)ncnc3cc2OC)CC1] | LC/MS: 634.3 [M + H]⁺. ¹H NMR (400 MHz, DMSO) δ 10.79 (s, 1H), 8.71 (s, 1H), 8.12 (d, J = 5.8 Hz, 1H), 7.63 (t, J = 8.8 Hz, 1H), 7.46 (s, 1H), 7.35 (dd, J = 10.9, 2.6 Hz, 1H), 7.21-7.11 (m, 2H), 6.87 (dd, J = 16.7, 10.5 Hz, 1H), 6.66 (s, 1H), 6.33 (dd, J = 5.7, 1.7 Hz, 1H), 6.13 (dd, J = 16.7, 2.4 Hz, 1H), 6.04 (d, J = 7.5 Hz, 1H), 5.70 (dd, J = 10.4, 2.4 Hz, 1H), 4.49 (d, J = 13.9 Hz, 1H), 4.16 (d, J = 14.3 Hz, 1H), 4.04 (s, 3H), 3.76 (dd, J = 8.7, 3.9 Hz, 1H), 3.73-3.65 (m, 4H), 3.21 (t, J = 12.5 Hz, 1H), 2.83 (t, J = 12.2 Hz, 1H), 2.12-1.92 (m, 6H), 1.56-1.42 (m, 2H). |
| 219 | 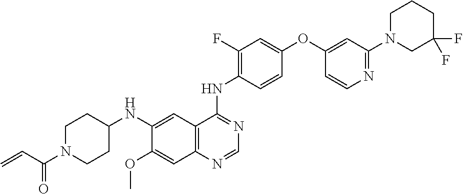 [SMILES: C=CC(=O)N1CCC(Nc2cc3c(Nc4ccc(Oc5ccnc(N6CCCC(F)(F)C6)c5)cc4F)ncnc3cc2OC)CC1] | LC/MS: 633.8 [M + H]⁺. ¹H NMR (400 MHz, DMSO) δ 9.19 (s, 1H), 8.23 (s, 1H), 8.06 (d, J = 5.7 Hz, 1H), 7.58 (t, J = 8.8 Hz, 1H), 7.26 (s, 1H), 7.22 (dd, J = 11.0, 2.7 Hz, 1H), 7.08 (s, 1H), 7.03 (dd, J = 8.7, 1.8 Hz, 1H), 6.86 (dd, J = 16.7, 10.5 Hz, 1H), 6.57 (d, J = 1.9 Hz, 1H), 6.26 (dd, J = 5.7, 2.0 Hz, 1H), 6.12 (dd, J = 16.7, 2.5 Hz, 1H), 5.69 (dd, J = 10.4, 2.5 Hz, 1H), 5.34 (d, J = 8.5 Hz, 1H), 4.46 (d, J = 12.4 Hz, 1H), 4.14 (d, J = 13.5 Hz, 1H), 3.96 (s, 3H), 3.92 (t, J = 12.2 Hz, 2H), 3.79-3.71 (m, 1H), 3.57-3.53 (m, 2H), 3.23 (t, J = 12.3 Hz, 1H), |
| | | 2.85 (t, J = 12.0 Hz, 1H), 2.17-2.02 (m, 4H), |
| | | 1.75-1.69 (m, 2H), 1.48-1.41 (m, 2H). |
| 220 | 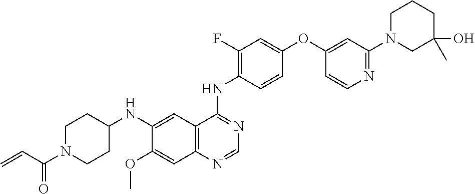 [SMILES: C=CC(=O)N1CCC(Nc2cc3c(Nc4ccc(Oc5ccnc(N6CCCC(C)(O)C6)c5)cc4F)ncnc3cc2OC)CC1] | LC/MS: 628.3 [M + H]⁺. ¹H NMR (400 MHz, DMSO) δ 8.32 (s, 1H), 8.01 (d, J = 5.7 Hz, 1H), 7.58 (t, J = 8.8 Hz, 1H), 7.37 (s, 1H), 7.23-7.19 (m, 1H), 7.10 (s, 1H), 7.03 (dd, J = 8.7, 1.8 Hz, 1H), 6.86 (dd, J = 16.7, 10.5 Hz, 1H), 6.38 (d, J = 1.8 Hz, 1H), 6.20- 6.17 (m, 1H), 6.12 (dd, J = 16.7, 2.4 Hz, 1H), 5.69 (dd, J = 10.4, 2.4 Hz, 1H), 5.48 (d, J = 8.5 Hz, 1H), 4.48-4.42 (m, 2H), 4.14 (d, J = 13.3 Hz, 1H), 3.98 (s, 3H), 3.81-3.76 (m, 1H), 3.58- 3.54 (m, 1H), 3.41-3.36 (m, 1H), 3.31-3.24 (m, 3H), 2.88 (t, J = 11.9 Hz, 1H), 2.12-2.03 (m, 1H), 1.75-1.69 (m, 1H), 1.57-1.52 (m, |
| | | 2H), 1.49-1.38 (m, 4H), 1.25-1.183 (m, 1H), |
| | | 1.10 (s, 3H). |
| 221 | 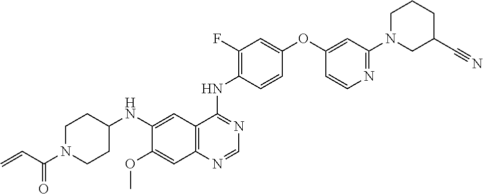 [SMILES: C=CC(=O)N1CCC(Nc2cc3c(Nc4ccc(Oc5ccnc(N6CCCC(C#N)C6)c5)cc4F)ncnc3cc2OC)CC1] | LC/MS: 623.1 [M + H]⁺. ¹H NMR (400 MHz,) δ 9.18 (s, 1H), 8.36 (brs, 1H), 8.22 (s, 1H), 8.08 (d, J = 5.6 Hz, 1H), 7.58 (t, J = 8.8 Hz, 1H), 7.26 (s, 1H), 7.21 (dd, J = 10.8, 2.8 Hz, 1H), 7.08 (s, 1H), 7.05-7.02 (m, 1H), 6.86 (dd, J = 16.6, 10.4 Hz, 1H), 6.56 (d, J = 2.0 Hz, 1H), 6.27 (dd, J = 5.6, 2.0 Hz, 1H), 6.12 (dd, J = 16.8, 2.4 Hz, 1H), 5.68 (dd, J = 10.4, 2.4 Hz, 1H), 5.33 (d, J = 8.4 Hz, 1H), 4.46 (dd, J = 16.4, 5.2 Hz, 1H), 4.15-4.11 (m, 1H), 3.96 (s, 3H), 3.86 (dd, J = 13.2, 6.8 Hz, 1H), 3.82-3.74 (m, 2H), 3.61-3.52 (m, 1H), 3.43 (d, J = 13.6 Hz, 2H), 3.14-3.02 (m, 1H), 2.86 |
| | | (t, J = 10.4 Hz, 1H), 2.13-2.04 (m, 2H), 1.97- |
| | | 1.78 (m, 2H), 1.74-1.56 (m, 2H), 1.51-1.41 |
| | | (m, 2H). |
| 222 | 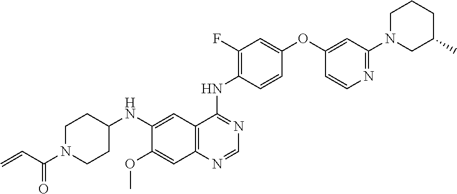 [SMILES: C=CC(=O)N1CCC(Nc2cc3c(Nc4ccc(Oc5ccnc(N6CCC[C@H](C)C6)c5)cc4F)ncnc3cc2OC)CC1] | LC/MS: 611.9 [M + H]⁺. ¹H NMR (400 MHz, DMSO) δ 9.17 (s, 1H), 8.22 (s, 1H), 8.04 (d, J = 5.7 Hz, 1H), 7.55 (d, J = 8.9 Hz, 1H), 7.25 (s, 1H), 7.20 (dd, J = 11.0, 2.7 Hz, 1H), 7.08 (s, 1H), 7.02 (dd, J = 8.5, 1.9 Hz, 1H), 6.86 (d, J = 6.2 Hz, 1H), 6.42 (d, J = 1.9 Hz, 1H), 6.18 (dd, J = 5.7, 2.0 Hz, 1H), 6.12 (dd, J = 16.7, 2.5 Hz, 1H), 5.69 (dd, J = 10.4, 2.4 Hz, 1H), 5.34 (d, J = 8.6 Hz, 1H), 4.46 (d, J = 12.0 Hz, 1H), 4.20-4.09 (m, 3H), 3.96 (s, 2H), 3.25- 3.19 (m, 1H), 2.85 (s, 1H), 2.79-2.73 (m, 1H), 2.58-2.54 (m, 1H), 2.43 (d, J = 12.7 Hz, 1H), 2.12-2.03 (m, 2H), 1.82-1.73 (m, 1H), 1.68- |
| | | 1.67 (m, 1H), 1.42 (d, J = 12.2 Hz, 3H), 1.32- |
| | | 1.21 (m, 2H), 1.17-1.09 (m, 1H), 0.89 (d, J = |
| | | 6.6 Hz, 3H). |
| 223 | 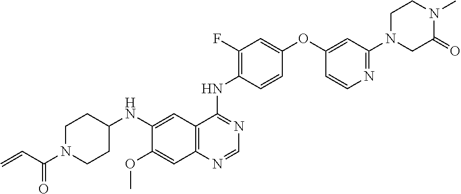 [SMILES: C=CC(=O)N1CCC(Nc2cc3c(Nc4ccc(Oc5ccnc(N6CCN(C)C(=O)C6)c5)cc4F)ncnc3cc2OC)CC1] | LC/MS: 627.2 [M + H]⁺. ¹H NMR (400 MHz, DMSO) δ 10.87 (s, 1H), 8.72 (s, 1H), 8.12 (d, J = 5.8 Hz, 1H), 7.65 (t, J = 8.7 Hz, 1H), 7.48 (s, 1H), 7.36 (dd, J = 10.8, 2.6 Hz, 1H), 7.23 (s, 1H), 7.15 (dd, J = 8.7, 1.9 Hz, 1H), 6.87 (dd, J = 16.7, 10.5 Hz, 1H), 6.49 (d, J = 1.7 Hz, 1H), 6.38 (dd, J = 5.8, 1.9 Hz, 1H), 6.16-5.99 (m, 2H), 5.70 (dd, J = 10.4, 2.4 Hz, 1H), 4.49 (d, J = 13.3 Hz, 1H), 4.16 (d, J = 13.4 Hz, 1H), 4.04 (d, J = 6.6 Hz, 5H), 3.86- 3.71 (m, 3H), 3.47-3.35 (m, 2H), 3.22 (t, J = 12.5 Hz, 1H), 2.93-2.74 (m, 4H), 2.09-2.00 (m, 2H), 1.55-1.40 (m, 2H). |
| 224 | 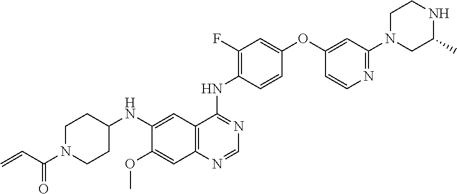 [SMILES: C=CC(=O)N1CCC(Nc2cc3c(Nc4ccc(Oc5ccnc(N6CCN[C@H](C)C6)c5)cc4F)ncnc3cc2OC)CC1] | LC/MS: 613.3 [M + H]⁺. ¹H NMR (400 MHz, DMSO) δ 9.22 (s, 1H), 8.22 (s, 1H), 8.08 (d, J = 5.7 Hz, 1H), 7.58 (t, J = 8.8 Hz, 1H), 7.49-7.09 (m, 3H), 7.09-6.99 (m, 2H), 6.91-6.81 (m, 1H), 6.54 (d, J = 1.8 Hz, 1H), 6.28 (dd, J = 5.7, 2.0 Hz, 1H), 6.16-6.08 (m, 1H), 5.69 (dd, J = 10.4, 2.5 Hz, 1H), 5.34 (d, J = 8.6 Hz, 1H), 4.50-4.42 (m, 1H), 4.27- 4.10 (m, 3H), 3.96 (s, 3H), 3.81-3.71 (m, 1H), 3.26-3.13 (m, 2H), 3.03-2.82 (m, 4H), 2.69- 2.60 (m, 1H), 2.12-2.03 (m, 2H), 1.49-1.38 (m, 2H), 1.25-1.12 (m, 3H). |
| 225 | 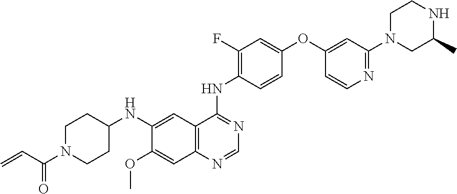 [SMILES: C=CC(=O)N1CCC(Nc2cc3c(Nc4ccc(Oc5ccnc(N6CCN[C@@H](C)C6)c5)cc4F)ncnc3cc2OC)CC1] | LC/MS: 613.3 [M + H]⁺. ¹H NMR (400 MHz, DMSO) δ 9.23 (s, 1H), 8.27- 8.03 (m, 2H), 7.58 (t, J = 8.8 Hz, 1H), 7.49- 7.09 (m, 3H), 7.09-6.99 (m, 2H), 6.91-6.82 (m, 1H), 6.55 (d, J = 1.8 Hz, 1H), 6.31-6.06 (m, 2H), 5.69 (dd, J = 10.4, 2.5 Hz, 1H), 5.34 (d, J = 8.6 Hz, 1H), 4.51-4.41 (m, 1H), 4.30- 4.09 (m, 3H), 3.96 (s, 3H), 3.83-3.72 (m, 1H), 3.27-3.15 (m, 2H), 3.06-2.82 (m, 4H), 2.73- 2.63 (m, 1H), 2.11-1.99 (m, 2H), 1.51-1.38 (m, 2H), 1.17 (d, J = 6.4 Hz, 3H). |
| 226 | 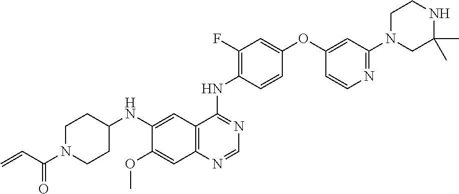 [SMILES: C=CC(=O)N1CCC(Nc2cc3c(Nc4ccc(Oc5ccnc(N6CCNC(C)(C)C6)c5)cc4F)ncnc3cc2OC)CC1] | LC/MS: 627.3 [M + H]⁺. ¹H NMR (400 MHz, DMSO) δ 10.93 (s, 1H), 9.15 (s, 2H), 8.71 (s, 1H), 8.11 (d, J = 5.7 Hz, 1H), 7.63 (t, J = 8.7 Hz, 1H), 7.51 (s, 1H), 7.36- 7.26 (m, 2H), 7.14-7.08 (m, 1H), 6.87 (dd, J = 16.6, 10.5 Hz, 1H), 6.71 (s, 1H), 6.32 (dd, J = 5.7, 1.6 Hz, 1H), 6.16-6.00 (m, 2H), 5.70 (dd, J = 10.4, 2.3 Hz, 1H), 4.48 (d, J = 12.1 Hz, 1H), 4.15 (d, J = 12.5 Hz, 1H), 4.04 (s, 3H), 3.90- 3.70 (m, 3H), 3.63 (s, 2H), 3.31-3.16 (m, 3H), 2.83 (t, J = 12.0 Hz, 1H), 2.08-1.98 (m, 2H), 1.60-1.40 (m, 2H), 1.31 (s, 6H). |
| 227 | 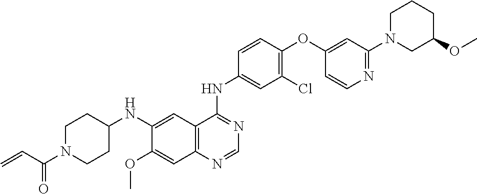 [SMILES: C=CC(=O)N1CCC(Nc2cc3c(Nc4ccc(Oc5ccnc(N6CCC[C@@H](OC)C6)c5)c(Cl)c4)ncnc3cc2OC)CC1] | LC/MS: 644.3 [M + H]⁺. ¹H NMR (400 MHz, DMSO) δ 10.91 (s, 1H), 8.81 (s, 1H), 8.09 (d, J = 2.5 Hz, 1H), 8.03 (d, J = 6.6 Hz, 1H), 7.80 (dd, J = 8.8, 2.5 Hz, 1H), 7.56-7.51 (m, 2H), 7.26 (s, 1H), 6.87 (dd, J = 16.7, 10.5 Hz, 1H), 6.69 (s, 1H), 6.33 (dd, J = 6.5, 1.7 Hz, 1H), 6.13 (dd, J = 16.7, 2.4 Hz, 1H), 6.03 (brs, 1H), 5.70 (dd, J = 10.4, 2.4 Hz, 1H), 4.47 (d, J = 11.9 Hz, 1H), 4.15 (d, J = 13.1 Hz, 1H), 4.05 (s, 3H), 3.89-3.75 (m, 2H), 3.64- 3.55 (m, 1H), 3.53-3.42 (m, 2H), 3.41-3.34 (m, 1H), 3.29-3.18 (m, 4H), 2.86 (t, J = 12.0 |
| | | Hz, 1H), 2.07-1.97 (m, 2H), 1.93-1.85 (m, |
| | | 1H), 1.77-1.68 (m, 1H), 1.73-1.67 (m, 1H), |
| | | 1.56-1.42 (m, 3H). |
| 228 | 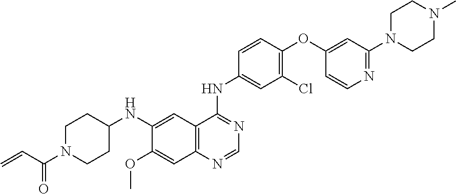 [SMILES: C=CC(=O)N1CCC(Nc2cc3c(Nc4ccc(Oc5ccnc(N6CCN(C)CC6)c5)c(Cl)c4)ncnc3cc2OC)CC1] | LC/MS: 629.1 [M + H]⁺. ¹H NMR (400 MHz, DMSO) δ 9.43 (s, 1H), 8.41 (s, 1H), 8.27-8.21 (m, 2H), 8.01 (d, J = 5.7 Hz, 1H), 7.91 (dd, J = 8.9, 2.4 Hz, 1H), 7.33 (t, J = 4.4 Hz, 2H), 7.11 (s, 1H), 6.86 (dd, J = 16.7, 10.5 Hz, 1H), 6.31 (d, J = 1.8 Hz, 1H), 6.15- 6.09 (m, 2H), 5.69 (dd, J = 10.4, 2.4 Hz, 1H), 5.38 (d, J = 8.7 Hz, 1H), 4.45 (d, J = 11.9 Hz, 1H), 4.13 (d, J = 12.5 Hz, 1H), 3.98 (s, 3H), 3.91-3.81 (m, 1H), 3.48-3.41 (m, 4H), 3.27 (t, J = 12.4 Hz, 1H), 2.92-2.87 (m, 1H), 2.40-2.35 (m, 4H), 2.21 (s, 3H), 2.12-2.01 (m, 2H), 1.52- 1.37 (m, 2H). |
| 229 | 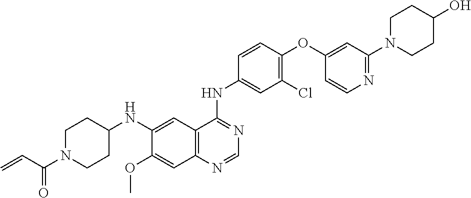 [SMILES: C=CC(=O)N1CCC(Nc2cc3c(Nc4ccc(Oc5ccnc(N6CCC(O)CC6)c5)c(Cl)c4)ncnc3cc2OC)CC1] | LC/MS: 630.2 [M + H]⁺. ¹H NMR (400 MHz, DMSO) δ 9.37 (s, 1H), 8.41 (s, 1H), 8.21 (s, 2H), 7.99 (d, J = 5.6 Hz, 1H), 7.89 (d, J = 8.7 Hz, 1H), 7.37-7.27 (m, 2H), 7.11 (s, 1H), 6.87 (dd, J = 16.6, 10.5 Hz, 1H), 6.30 (s, 1H), 6.16-6.04 (m, 2H), 5.69 (dd, J = 10.4, 2.1 Hz, 1H), 5.39 (d, J = 8.6 Hz, 1H), 4.45 (d, J = 12.9 Hz, 1H), 4.13 (d, J = 11.9 Hz, 1H), 3.98 (s, 3H), 3.93-3.91 (m, 1H), 3.89-3.77 (m, 2H), 3.71-3.62 (m, 1H), 3.26 (t, J = 13.2 Hz, 1H), 3.03 (t, J = 10.4 Hz, 2H), 2.90 (t, J = 12.8 Hz, 1H), 2.13-2.01 (m, 2H), 1.80-1.70 (m, 2H), 1.53-1.39 (m, 2H), 1.36-1.26 (m, 2H). |
| 230 | 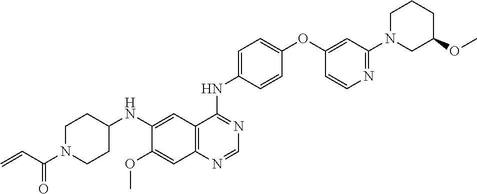 [SMILES: C=CC(=O)N1CCC(Nc2cc3c(Nc4ccc(Oc5ccnc(N6CCC[C@@H](OC)C6)c5)cc4)ncnc3cc2OC)CC1] | LC/MS: 610.4 [M + H]⁺. ¹H NMR (400 MHz, DMSO) δ 9.27 (s, 1H), 8.32 (s, 1H), 8.21 (s, 1H), 7.99 (d, J = 5.6 Hz, 1H), 7.83 (d, J = 8.0 Hz, 2H), 7.31 (s, 1H), 7.17 (d, J = 8.0 Hz, 2H), 7.08 (s, 1H), 6.86 (dd, J = 16.8, 10.4 Hz, 1H), 6.32 (d, J = 2.0 Hz, 1H), 6.17- 6.11 (m, 2H), 5.69 (dd, J = 10.4, 2.4 Hz, 1H), 5.33 (d, J = 8.8 Hz, 1H), 4.45 (d, J = 12.8 Hz, 1H), 4.13 (d, J = 14.4 Hz, 1H), 3.99 (d, J = 17.6 Hz, 4H), 3.83 (d, J = 8.0 Hz, 1H), 3.68 (d, J = 16.0 Hz, 1H), 3.27 (s, 3H), 3.23 (m, 1H), 3.08 (m, 1H), 2.89 (t, J = 12.4 Hz, 1H), 2.17-2.06 (m, 1H), 2.01-1.92 (m, 1H), 1.75-1.63 (m, |
| | | 1H), 1.52-1.41 (m, 4H). |
| 231 | 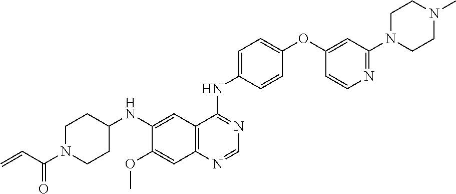 [SMILES: C=CC(=O)N1CCC(Nc2cc3c(Nc4ccc(Oc5ccnc(N6CCN(C)CC6)c5)cc4)ncnc3cc2OC)CC1] | LC/MS: 595.2 [M + H]⁺. ¹H NMR (400 MHz, DMSO) δ 8.54 (s, 1H), 8.09 (d, J = 5.6 Hz, 1H), 7.84 (d, J = 8.0 Hz, 2H), 7.66 (s, 1H), 7.25-7.17 (m, 3H), 6.86 (dd, J = 16.8, 10.4 Hz, 1H), 6.58 (s, 1H), 6.28 (dd, J = 5.6, 2.0 Hz, 1H), 6.12 (dd, J = 16.8, 2.4 Hz, 1H), 5.69 (dd, J = 10.4, 2.4 Hz, 1H), 4.52-4.26 (m, 3H), 4.13 (d, J = 13.6 Hz, 1H), 4.00 (s, 3H), 3.27 (d, J = 11.2 Hz, 1H), 3.17-3.04 (m, 4H), 2.99- 2.92 (m, 1H), 2.83-2.75 (m, 2H), 2.13-2.02 (m, 2H), 1.52-1.41 (m, 1H), 1.27-1.12 (m, 5H). |
| 234 | 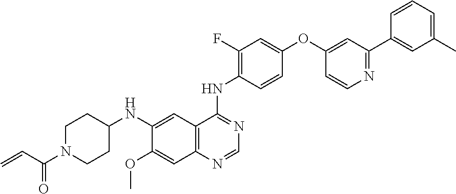 [SMILES: C=CC(=O)N1CCC(Nc2cc3c(Nc4ccc(Oc5ccnc(-c6cccc(C)c6)c5)cc4F)ncnc3cc2OC)CC1] | LC/MS: 604.8 [M + H]⁺. 1H NMR (400 MHz, DMSO) δ 9.57 (brs, 1H), 8.61 (d, J = 5.6 Hz, 1H), 8.33 (s, 1H), 7.91 (s, 1H), 7.83 (d, J = 7.8 Hz, 1H), 7.64 (t, J = 8.8 Hz, 1H), 7.60 (d, J = 2.3 Hz, 1H), 7.42-7.33 (m, 3H), 7.27 (d, J = 7.5 Hz, 1H), 7.16 (dd, J = 8.7, 1.8 Hz, 1H), 7.10 (s, 1H), 6.94 (dd, J = 5.6, 2.3 Hz, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.47 (d, J = 8.5 Hz, 1H), 4.47 (d, J = 12.2 Hz, 1H), 4.14 (d, J = 13.7 Hz, 1H), 3.98 (s, 3H), 3.83-3.74 (m, 1H), 3.24 (t, J = 12.1 Hz, 1H), 2.86 (t, J = 11.6 Hz, 1H), 2.39 (s, 3H), 2.13- |
| | | 2.03 (m, 2H), 1.51-1.39 (m, 2H). |
| 235 | 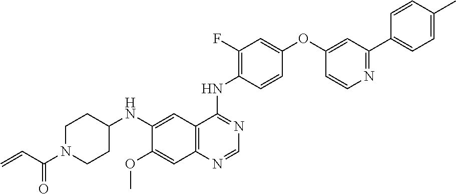 [SMILES: C=CC(=O)N1CCC(Nc2cc3c(Nc4ccc(Oc5ccnc(-c6ccc(C)cc6)c5)cc4F)ncnc3cc2OC)CC1] | LC/MS: 605.3 [M + H]⁺. ¹H NMR (400 MHz, DMSO) δ 9.21 (s, 1H), 8.58 (d, J = 5.6 Hz, 1H), 8.24 (s, 1H), 7.96 (d, J = 8.0 Hz, 2H), 7.64 (t, J = 8.8 Hz, 1H), 7.55 (d, J = 2.4 Hz, 1H), 7.35 (dd, J = 10.8, 2.8 Hz, 1H), 7.32-7.26 (m, 3H), 7.14 (dd, J = 8.8, 1.6 Hz, 1H), 7.09 (s, 1H), 6.93-6.90 (m, 1H), 6.89- 6.82 (m, 1H), 6.12 (dd, J = 16.8, 2.4 Hz, 1H), 5.69 (dd, J = 10.4, 2.4 Hz, 1H), 5.34 (d, J = 8.4 Hz, 1H), 4.47 (d, J = 12.4 Hz, 1H), 4.14 (d, J = 14.0 Hz, 1H), 3.97 (s, 3H), 3.77 (s, 1H), 3.22 (d, J = 12.0 Hz, 1H), 2.85 (d, J = 11.6 Hz, 1H), 2.36 (s, 3H), 2.11-2.03 (m, 2H), 1.52-1.43 (m, 2H). |
| 236 | 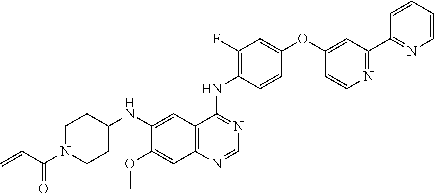 [SMILES: C=CC(=O)N1CCC(Nc2cc3c(Nc4ccc(Oc5ccnc(-c6ccccn6)c5)cc4F)ncnc3cc2OC)CC1] | LC/MS: 592.2 [M + H]⁺. ¹H NMR (400 MHz, DMSO) δ 10.85 (s, 1H), 8.81-8.60 (m, 3H), 8.42 (d, J = 7.9 Hz, 1H), 8.05-7.90 (m, 2H), 7.73 (t, J = 8.7 Hz, 1H), 7.60-7.40 (m, 3H), 7.32-7.13 (m, 3H), 6.88 (dd, J = 16.7, 10.5 Hz, 1H), 6.17-6.02 (m, 2H), 5.70 (dd, J = 10.4, 2.5 Hz, 1H), 4.50 (d, J = 13.0 Hz, 1H), 4.17 (d, J = 13.1 Hz, 1H), 4.05 (s, 3H), 3.85-3.74 (m, 1H), 3.23 (t, J = 12.4 Hz, 1H), 2.85 (t, J = 12.8 Hz, 1H), 2.15-2.03 (m, 2H), 1.60-1.40 (m, 2H). |
| 237 | 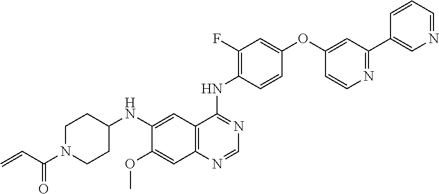 [SMILES: C=CC(=O)N1CCC(Nc2cc3c(Nc4ccc(Oc5ccnc(-c6cccnc6)c5)cc4F)ncnc3cc2OC)CC1] | LC/MS: 592.3 [M + H]⁺. ¹H NMR (400 MHz, DMSO) δ 9.30-9.20 (m, 2H), 8.65 (d, J = 5.6 Hz, 2H), 8.46-8.40 (m, 1H), 8.24 (s, 1H), 7.77 (d, J = 2.3 Hz, 1H), 7.65 (t, J = 8.8 Hz, 1H), 7.54 (dd, J = 7.8, 4.6 Hz, 1H), 7.37 (dd, J = 10.8, 2.6 Hz, 1H), 7.27 (s, 1H), 7.16 (dd, J = 8.6, 1.9 Hz, 1H), 7.09 (s, 1H), 6.98 (dd, J = 5.6, 2.3 Hz, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.35 (d, J = 8.5 Hz, 1H), 4.46 (d, J = 13.2 Hz, 1H), 4.14 (d, J = 13.5 Hz, 1H), 3.97 (s, 3H), 3.80-3.70 (m, 1H), |
| | | 3.25-3.20 (m, 1H), 2.86 (t, J = 11.9 Hz, 1H), |
| | | 2.15-2.00 (m, 2H), 1.48-1.38 (m, 2H). |
| 238 | 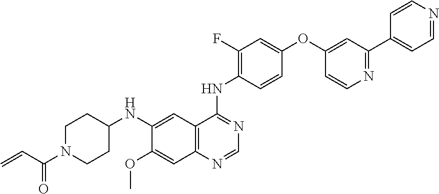 [SMILES: C=CC(=O)N1CCC(Nc2cc3c(Nc4ccc(Oc5ccnc(-c6ccncc6)c5)cc4F)ncnc3cc2OC)CC1] | LC/MS: 592.2 [M + H]⁺. ¹H NMR (400 MHz, DMSO) δ 9.23 (s, 1H), 8.75- 8.65 (m, 3H), 8.24 (s, 1H), 8.05 (dd, J = 4.5, 1.6 Hz, 2H), 7.83 (d, J = 2.3 Hz, 1H), 7.65 (t, J = 8.8 Hz, 1H), 7.38 (dd, J = 10.9, 2.7 Hz, 1H), 7.28 (s, 1H), 7.16 (dd, J = 8.6, 1.8 Hz, 1H), 7.09 (s, 1H), 7.04 (dd, J = 5.6, 2.4 Hz, 1H), 6.87 (dd, J = 16.7, 10.5 Hz, 1H), 6.12 (dd, J = 16.7, 2.5 Hz, 1H), 5.69 (dd, J = 10.4, 2.5 Hz, 1H), 5.35 (d, J = 8.6 Hz, 1H), 4.47 (d, J = 12.8 Hz, 1H), 4.14 (d, J = 12.5 Hz, 1H), 3.97 (s, 3H), 3.80- 3.70 (m, 1H), 3.25-3.20 (m, 1H), 2.86 (t, J = |
| | | 11.7 Hz, 1H), 2.15-2.00 (m, 2H), 1.50-1.40 |
| | | (m, 2H). |
| 239 | 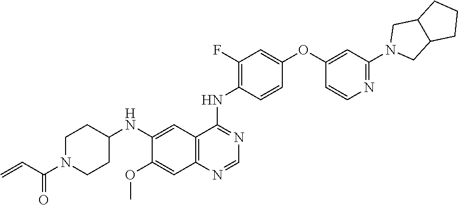 [SMILES: C=CC(=O)N1CCC(Nc2cc3c(Nc4ccc(Oc5ccnc(N6CC7CCCC7C6)c5)cc4F)ncnc3cc2OC)CC1] | LC/MS: 624.3 [M + H]⁺. ¹H NMR (400 MHz, DMSO) δ 9.18 (s, 1H), 8.21 (d, J = 11.3 Hz, 2H), 8.01 (d, J = 5.7 Hz, 1H), 7.57 (t, J = 8.8 Hz, 1H), 7.34-7.13 (m, 2H), 7.12-6.95 (m, 2H), 6.86 (dd, J = 16.7, 10.5 Hz, 1H), 6.21 (dd, J = 5.7, 2.1 Hz, 1H), 6.12 (dd, J = 16.7, 2.5 Hz, 1H), 6.01 (d, J = 2.1 Hz, 1H), 5.69 (dd, J = 10.4, 2.5 Hz, 1H), 5.34 (d, J = 8.6 Hz, 1H), 4.46 (d, J = 13.2 Hz, 1H), 4.14 (d, J = 12.4 Hz, 1H), 3.96 (s, 3H), 3.80-3.70 (m, 1H), 3.54 (dd, J = 10.5, 7.9 Hz, 2H), 3.23 (t, J = 12.1 Hz, 1H), 3.12 (dd, J = 10.7, 3.7 Hz, 2H), 2.85 (t, J = 12.2 Hz, 1H), 2.78-2.68 (m, 2H), 2.16- |
| | | 1.98 (m, 2H), 1.86-1.74 (m, 2H), 1.74-1.63 |
| | | (m, 1H), 1.62-1.31 (m, 5H). |
| 240 | 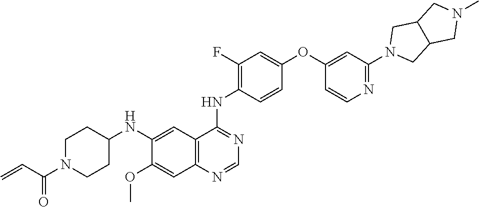 [SMILES: C=CC(=O)N1CCC(Nc2cc3c(Nc4ccc(Oc5ccnc(N6CC7CN(C)CC7C6)c5)cc4F)ncnc3cc2OC)CC1] | LC/MS: 639.3 [M + H]⁺. ¹H NMR (400 MHz, DMSO) δ 9.20 (brs, 1H), 8.22 (s, 1H), 8.19 (s, 2H), 8.04 (d, J = 5.7 Hz, 1H), 7.58 (t, J = 8.8 Hz, 1H), 7.27 (s, 1H), 7.20 (dd, J = 11.0, 2.6 Hz, 1H), 7.08 (s, 1H), 7.03 (dd, J = 8.7, 1.9 Hz, 1H), 6.86 (dd, J = 16.7, 10.5 Hz, 1H), 6.25 (dd, J = 5.7, 2.1 Hz, 1H), 6.12 (dd, J = 16.7, 2.5 Hz, 1H), 6.06 (d, J = 2.0 Hz, 1H), 5.69 (dd, J = 10.4, 2.4 Hz, 1H), 5.34 (d, J = 8.5 Hz, 1H), 4.46 (d, J = 12.2 Hz, 1H), 4.14 (d, J = 13.5 Hz, 1H), 3.96 (s, 3H), 3.81- 3.70 (m, 1H), 3.50 (dd, J = 10.5, 7.9 Hz, 2H), 3.31-3.16 (m, 3H), 2.98-2.90 (m, 2H), 2.86 (t, |
| | | J = 11.9 Hz, 1H), 2.76-2.66 (m, 2H), 2.57 (dd, |
| | | J = 9.5, 2.9 Hz, 2H), 2.32 (d, J = 9.1 Hz, 3H), |
| | | 2.12-2.02 (m, 2H), 1.53-1.38 (m, 2H). |
| 241 | 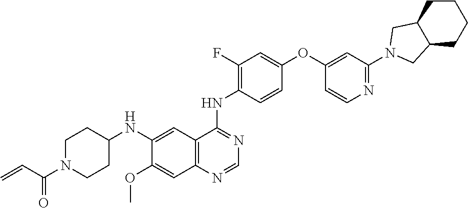 [SMILES: C=CC(=O)N1CCC(Nc2cc3c(Nc4ccc(Oc5ccnc(N6C[C@H]7CCCC[C@H]7C6)c5)cc4F)ncnc3cc2OC)CC1] | LC/MS: 638.3 [M + H]⁺. ¹H NMR (400 MHz, DMSO) δ 9.18 (s, 1H), 8.22 (s, 1H), 8.17 (s, 1H), 8.00 (d, J = 5.7 Hz, 1H), 7.57 (t, J = 8.8 Hz, 1H), 7.32-7.16 (m, 2H), 7.11-6.98 (m, 2H), 6.86 (dd, J = 16.7, 10.5 Hz, 1H), 6.18 (dd, J = 5.7, 2.1 Hz, 1H), 6.12 (dd, J = 16.7, 2.4 Hz, 1H), 5.94 (d, J = 2.0 Hz, 1H), 5.69 (dd, J = 10.4, 2.5 Hz, 1H), 5.34 (d, J = 8.5 Hz, 1H), 4.46 (d, J = 13.5 Hz, 1H), 4.14 (d, J = 12.8 Hz, 1H), 3.94 (s, 3H), 3.80-3.70 (m, 1H), 3.34-3.10 (m, 5H), 2.85 (t, J = 11.5 Hz, 1H), 2.34-2.20 (m, 2H), 2.14-2.00 (m, 2H), 1.65- 1.25 (m, 10H). |
| 242 | 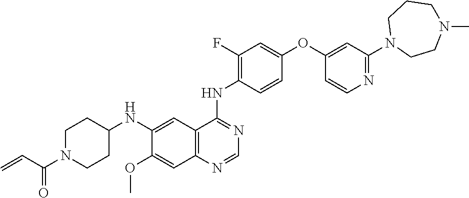 [SMILES: C=CC(=O)N1CCC(Nc2cc3c(Nc4ccc(Oc5ccnc(N6CCCN(C)CC6)c5)cc4F)ncnc3cc2OC)CC1] | LC/MS: 627.3 [M + H]⁺. ¹H NMR (400 MHz, DMSO) δ 9.18 (s, 1H), 8.25- 8.15 (m, 2H), 8.02 (d, J = 6.1 Hz, 1H), 7.57 (t, J = 8.8 Hz, 1H), 7.30-7.16 (m, 2H), 7.12-6.97 (m, 2H), 6.86 (dd, J = 16.7, 10.5 Hz, 1H), 6.26- 6.03 (m, 3H), 5.69 (dd, J = 10.4, 2.4 Hz, 1H), 5.34 (d, J = 8.6 Hz, 1H), 4.46 (d, J = 13.0 Hz, 1H), 4.14 (d, J = 12.2 Hz, 1H), 3.96 (s, 3H), 3.80-3.70 (m, 3H), 3.52 (t, J = 6.0 Hz, 2H), 3.26-3.19 (m, 1H), 2.90-2.81 (m, 1H), 2.70-2.55 (m, 4H), 2.40-2.20 (m, 3H), 2.15-2.00 (m, 2H), 1.95-1.80 (m, 2H), 1.55-1.35 (m, 2H). |
| 244 | 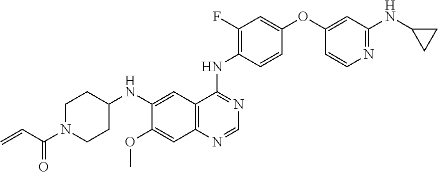 [SMILES: C=CC(=O)N1CCC(Nc2cc3c(Nc4ccc(Oc5ccnc(NC6CC6)c5)cc4F)ncnc3cc2OC)CC1] | LC/MS: 570.1 [M + H]⁺. ¹H NMR (400 MHz, DMSO) δ 10.92 (s, 1H), 9.01 (s, 1H), 8.71 (s, 1H), 8.07 (d, J = 7.2 Hz, 1H), 7.75 (t, J = 8.8 Hz, 1H), 7.57 (dd, J = 10.4, 2.4 Hz, 1H), 7.50 (s, 1H), 7.31 (dd, J = 8.8, 2.0 Hz, 1H), 7.25 (s, 1H), 6.87 (dd, J = 16.8, 10.4 Hz, 1H), 6.72 (d, J = 7.2 Hz, 1H), 6.33 (s, 1H), 6.13 (dd, J = 16.8, 2.4 Hz, 1H), 6.03 (s, 1H), 5.70 (dd, J = 10.4, 2.4 Hz, 1H), 4.48 (d, J = 12.4 Hz, 1H), 4.15 (d, J = 12.8 Hz, 1H), 4.05 (s, 3H), 3.82-3.76 (m, 1H), 3.25-3.18 (m, 1H), 2.88- 2.80 (m, 1H), 2.75-2.65 (m, 1H), 2.04 (d, J = |
| | | 10.8 Hz, 2H), 1.57-1.44 (m, 2H), 0.92-0.84 |
| | | (m, 2H), 0.63-0.55 (m, 2H). |
| 245 | 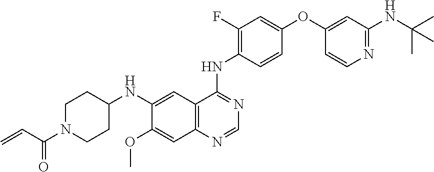 [SMILES: C=CC(=O)N1CCC(Nc2cc3c(Nc4ccc(Oc5ccnc(NC(C)(C)C)c5)cc4F)ncnc3cc2OC)CC1] | LC/MS: 586.1 [M + H]⁺. ¹H NMR (400 MHz, DMSO) δ 9.60 (s, 1H), 8.33 (s, 1H), 7.92 (d, J = 5.8 Hz, 1H), 7.59 (t, J = 8.8 Hz, 1H), 7.35-6.83 (m, 5H), 6.40 (s, 1H), 6.24- 6.03 (m, 3H), 5.69 (dd, J = 10.4, 2.5 Hz, 1H), 5.48 (d, J = 8.5 Hz, 1H), 4.51-4.41 (m, 1H), 4.19-4.09 (m, 1H), 3.98 (s, 3H), 3.84-3.72 (m, 1H), 3.29-3.20 (m, 1H), 2.92-2.79 (m, 1H), 2.12-2.01 (m, 2H), 1.51-1.33 (m, 11H). |
| 246 | 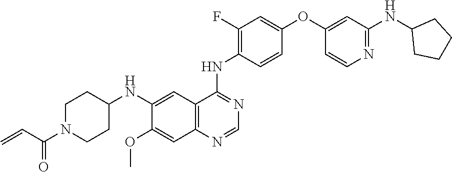 [SMILES: C=CC(=O)N1CCC(Nc2cc3c(Nc4ccc(Oc5ccnc(NC6CCCC6)c5)cc4F)ncnc3cc2OC)CC1] | LC/MS: 598.3 [M + H]⁺. ¹H NMR (400 MHz, DMSO) δ 9.18 (s, 1H), 8.23 (s, 1H), 7.91 (d, J = 5.6 Hz, 1H), 7.58 (t, J = 8.8 Hz, 1H), 7.26 (s, 1H), 7.21 (d, J = 8.8 Hz, 1H), 7.08 (s, 1H), 7.03 (d, J = 8.0 Hz, 1H), 6.86 (dd, J = 16.8, 10.4 Hz, 1H), 6.61 (d, J = 6.8 Hz, 1H), 6.21-6.05 (m, 2H), 5.97 (s, 1H), 5.69 (d, J = 10.4 Hz, 1H), 5.34 (d, J = 8.4 Hz, 1H), 4.46 (d, J = 12.0 Hz, 1H), 4.17-4.04 (m, 2H), 3.96 (s, 3H), 3.81-3.70 (m, 1H), 3.25-3.16 (m, 1H), 2.87 (t, J = 13.2 Hz, 1H), 2.13-2.01 (m, 2H), 1.94-1.80 (m, 2H), 1.69-1.58 (m, 2H), 1.57- |
| | | 1.32 (m, 6H). |
| 247 | 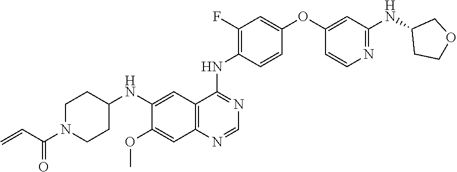 [SMILES: C=CC(=O)N1CCC(Nc2cc3c(Nc4ccc(Oc5ccnc(N[C@H]6CCOC6)c5)cc4F)ncnc3cc2OC)CC1] | LC/MS: 599.8 [M + H]⁺. 1H NMR (400 MHz, DMSO) δ 9.20 (s, 1H), 8.23 (s, 1H), 7.93 (d, J = 5.8 Hz, 1H), 7.58 (t, J = 8.8 Hz, 1H), 7.28-7.19 (m, 2H), 7.10-6.99 (m, 2H), 6.90-6.81 (m, 2H), 6.23 (dd, J = 5.8, 2.2 Hz, 1H), 6.12 (dd, J = 16.7, 2.4 Hz, 1H), 6.02 (d, J = 2.2 Hz, 1H), 5.69 (dd, J = 10.4, 2.5 Hz, 1H), 5.34 (d, J = 8.5 Hz, 1H), 4.46 (d, J = 14.2 Hz, 1H), 4.34 (s, 1H), 4.13 (d, J = 12.5 Hz, 1H), 3.96 (s, 3H), 3.86-3.67 (m, 4H), 3.50-3.45 (m, 1H), 3.28-3.16 (m, 1H), 2.86 (t, J = 11.8 Hz, |
| | | 1H), 2.17-2.01 (m, 3H), 1.79-1.70 (m, 1H), |
| | | 1.49-1.38 (m, 2H). |
| 248 | 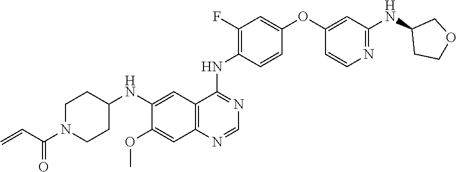 [SMILES: C=CC(=O)N1CCC(Nc2cc3c(Nc4ccc(Oc5ccnc(N[C@@H]6CCOC6)c5)cc4F)ncnc3cc2OC)CC1] | LC/MS: 599.9 [M + H]⁺. ¹H NMR (400 MHz, DMSO) δ 9.19 (s, 1H), 8.23 (s, 1H), 7.94 (d, J = 5.8 Hz, 1H), 7.58 (t, J = 8.8 Hz, 1H), 7.29-7.18 (m, 2H), 7.10-7.01 (m, 2H), 6.91-6.82 (m, 2H), 6.23 (dd, J = 5.8, 2.2 Hz, 1H), 6.12 (dd, J = 16.7, 2.5 Hz, 1H), 6.02 (d, J = 2.2 Hz, 1H), 5.69 (dd, J = 10.4, 2.5 Hz, 1H), 5.34 (d, J = 8.6 Hz, 1H), 4.46 (d, J = 12.5 Hz, 1H), 4.35 (d, J = 7.1 Hz, 1H), 4.13 (d, J = 13.1 Hz, 1H), 3.96 (s, 3H), 3.93-3.72 (m, 4H), 3.48 (dd, J = 8.8, 3.9 Hz, 1H), 3.21 (d, J = 12.3 |
| | | Hz, 1H), 2.86 (t, J = 11.5 Hz, 1H), 2.18-2.03 |
| | | (m, 3H), 1.82-1.73 (m, 1H), 1.52-1.39 (m, |
| | | 2H). |
| 249 | 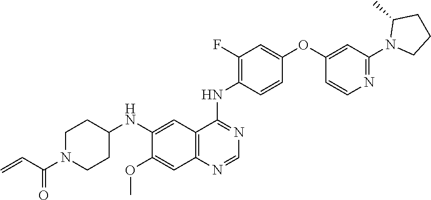 [SMILES: C=CC(=O)N1CCC(Nc2cc3c(Nc4ccc(Oc5ccnc(N6CCC[C@H]6C)c5)cc4F)ncnc3cc2OC)CC1] | LC/MS: 598.1 [M + H]⁺. ¹H NMR (400 MHz, DMSO) δ 10.98-10.89 (m, 1H), 8.72 (s, 1H), 8.09-8.04 (m, 1H), 7.72 (t, J = 8.7 Hz, 1H), 7.51 (t, J = 10.9 Hz, 2H), 7.31- 7.22 (m, 2H), 6.87 (dd, J = 16.7, 10.5 Hz, 1H), 6.58-6.51 (m, 1H), 6.42 (s, 1H), 6.18-6.00 (m, 2H), 5.70 (dd, J = 10.5, 2.3 Hz, 1H), 4.48 (d, J = 11.9 Hz, 1H), 4.16 (d, J = 13.8 Hz, 2H), 4.05 (s, 3H), 3.82-3.75 (m, 1H), 3.63-3.54 (m, 1H), 3.41-3.31 (m, 1H), 3.25-3.15 (m, 1H), 2.84 (t, J = 12.0 Hz, 1H), 2.19-1.98 (m, 5H), 1.77 (d, J = 9.4 Hz, 1H), 1.56-1.42 (m, 2H), 1.14 (d, J = 6.3 Hz, 3H). |
| 250 | 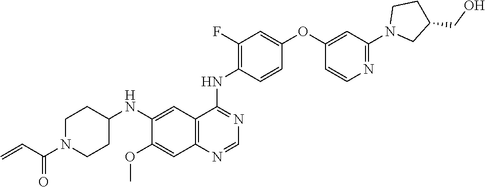 [SMILES: C=CC(=O)N1CCC(Nc2cc3c(Nc4ccc(Oc5ccnc(N6CC[C@H](CO)C6)c5)cc4F)ncnc3cc2OC)CC1] | LC/MS: 614.3 [M + H]⁺. ¹H NMR (400 MHz, DMSO) δ 9.20 (s, 1H), 8.22 (s, 1H), 8.01 (d, J = 5.6 Hz, 1H), 7.58 (t, J = 8.7 Hz, 1H), 7.28-7.19 (m, 2H), 7.09-7.02 (m, 2H), 6.86 (dd, J = 16.6, 10.5 Hz, 1H), 6.16 (dd, J = 36.5, 10.5 Hz, 2H), 5.93 (s, 1H), 5.69 (d, J = 10.2 Hz, 1H), 5.34 (d, J = 8.5 Hz, 1H), 4.72 (s, 1H), 4.47 (d, J = 12.1 Hz, 1H), 4.14 (d, J = 12.0 Hz, 1H), 3.96 (s, 3H), 3.81-3.71 (m, 1H), 3.41 (d, J = 8.4 Hz, 3H), 3.34-3.16 (m, 3H), 3.12-3.07 (m, 1H), 2.86 (t, J = 12.0 Hz, 1H), 2.42-2.36 (m, 1H), 2.09-1.96 (m, 3H), 1.76- |
| | | 1.66 (m, 1H), 1.48-1.37 (m, 2H). |
| 251 | 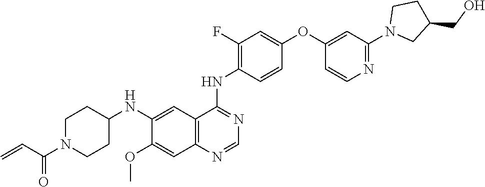 [SMILES: C=CC(=O)N1CCC(Nc2cc3c(Nc4ccc(Oc5ccnc(N6CC[C@@H](CO)C6)c5)cc4F)ncnc3cc2OC)CC1] | LC/MS: 614.1 [M + H]⁺. ¹H NMR (400 MHz,) δ 9.19 (s, 1H), 8.31 (s, 1H), 8.22 (s, 1H), 8.01 (d, J = 5.7 Hz, 1H), 7.57 (t, J = 8.8 Hz, 1H), 7.27-7.18 (m, 2H), 7.08- 7.01 (m, 2H), 6.86 (dd, J = 16.7, 10.5 Hz, 1H), 6.16 (ddd, J = 19.1, 11.2, 2.2 Hz, 2H), 5.93 (d, J = 1.9 Hz, 1H), 5.68 (dd, J = 10.4, 2.4 Hz, 1H), 5.33 (d, J = 8.5 Hz, 1H), 4.46 (d, J = 12.3 Hz, 1H), 4.13 (d, J = 13.9 Hz, 1H), 3.96 (s, 3H), 3.78-3.72 (m, 1H), 3.46-3.39 (m, 4H), 3.34- 3.30 (m, 2H), 3.24-3.20 (m, 1H), 3.15-3.02 (m, 1H), 2.85 (t, J = 12.1 Hz, 1H), 2.42-2.36 (m, |
| | | 1H), 2.10-1.97 (m, 3H), 1.75-1.67 (m, 1H), |
| | | 1.48-1.38 (m, 2H). |
| 252 | 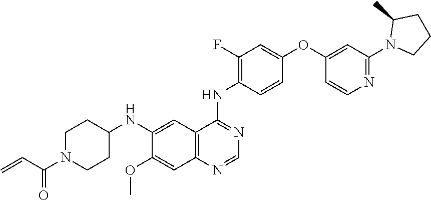 [SMILES: C=CC(=O)N1CCC(Nc2cc3c(Nc4ccc(Oc5ccnc(N6CCC[C@@H]6C)c5)cc4F)ncnc3cc2OC)CC1] | LC/MS: 598.0 [M + H]⁺. ¹H NMR (400 MHz, DMSO) δ 10.98-10.85 (m, 1H), 8.71 (s, 1H), 8.08-8.03 (m, 1H), 7.71 (t, J = 8.6 Hz, 1H), 7.50 (t, J = 10.1 Hz, 2H), 7.31- 7.21 (m, 2H), 6.87 (dd, J = 16.7, 10.5 Hz, 1H), 6.52-6.43 (m, 2H), 6.16-6.01 (m, 2H), 5.70 (dd, J = 10.4, 2.3 Hz, 1H), 4.48 (d, J = 12.3 Hz, 1H), 4.23-4.12 (m, 2H), 4.05 (s, 3H), 3.78 (s, 1H), 3.58 (s, 1H), 3.39-3.30 (m, 1H), 3.24- 3.16 (m, 1H), 2.84 (t, J = 12.2 Hz, 1H), 2.15- 1.96 (m, 5H), 1.76 (d, J = 7.9 Hz, 1H), 1.56- 1.41 (m, 2H), 1.14 (d, J = 6.3 Hz, 3H). |
| 253 | 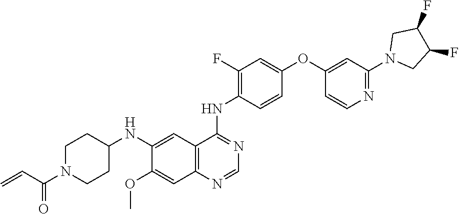 [SMILES: C=CC(=O)N1CCC(Nc2cc3c(Nc4ccc(Oc5ccnc(N6C[C@@H](F)[C@@H](F)C6)c5)cc4F)ncnc3cc2OC)CC1] | LC/MS: 620.0 [M + H]⁺. ¹H NMR (400 MHz,) δ 9.29 (s, 1H), 8.26 (s, 1H), 8.14 (s, 1H), 8.07 (d, J = 5.6 Hz, 1H), 7.58 (t, J = 8.8 Hz, 1H), 7.28 (s, 1H), 7.22 (dd, J = 11.2, 2.8 Hz, 1H), 7.08 (s, 1H), 7.06-7.02 (m, 1H), 6.86 (dd, J = 16.8, 10.4 Hz, 1H), 6.28 (dd, J = 5.6, 2.0 Hz, 1H), 6.16-6.09 (m, 2H), 5.68 (dd, J = 10.4, 2.4 Hz, 1H), 5.52-5.45 (m, 1H), 5.38-5.33 (m, 2H), 4.46 (d, J = 12.4 Hz, 1H), 4.13 (d, J = 12.8 Hz, 1H), 3.97 (s, 3H), 3.84- 3.75 (m, 3H), 3.61-3.52 (m, 2H), 3.26-3.19 (m, 1H), 2.86 (t, J = 12.0 Hz, 1H), 2.09-2.02 (m, 2H), 1.49-1.40 (m, 2H). |
| 254 | 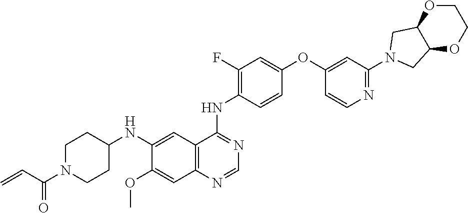 [SMILES: C=CC(=O)N1CCC(Nc2cc3c(Nc4ccc(Oc5ccnc(N6C[C@@H]7OCCO[C@@H]7C6)c5)cc4F)ncnc3cc2OC)CC1] | LC/MS: 642.2 [M + H]⁺. ¹H NMR (400 MHz,) δ 9.30 (s, 1H), 8.25 (s, 1H), 8.03 (d, J = 5.8 Hz, 1H), 7.57 (t, J = 8.8 Hz, 1H), 7.29-7.19 (m, 2H), 7.08 (s, 1H), 7.04 (ddd, J = 8.8, 2.4, 0.8 Hz, 1H), 6.86 (dd, J = 16.8, 10.4 Hz, 1H), 6.23 (dd, J = 5.6, 2.0 Hz, 1H), 6.12 (dd, J = 16.8, 2.4 Hz, 1H), 6.05 (d, J = 2.0 Hz, 1H), 5.68 (dd, J = 10.4, 2.4 Hz, 1H), 5.38 (d, J = 8.4 Hz, 1H), 4.46 (d, J = 12.8 Hz, 1H), 4.27 (t, J = 4.0 Hz, 2H), 4.13 (d, J = 12.0 Hz, 1H), 3.97 (s, 3H), 3.82-3.72 (m, 3H), 3.58- 3.51 (m, 4H), 3.48-3.42 (m, 2H), 3.24 (d, J = 13.2 Hz, 1H), 2.91-2.81 (m, 1H), 2.14-2.02 (m, 2H), 1.49-1.39 (m, 2H). |
| 255 | 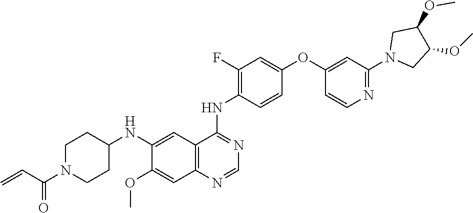 [SMILES: C=CC(=O)N1CCC(Nc2cc3c(Nc4ccc(Oc5ccnc(N6C[C@@H](OC)[C@H](OC)C6)c5)cc4F)ncnc3cc2OC)CC1] | LC/MS: 644.3 [M + H]⁺. ¹H NMR (400 MHz, DMSO) δ 9.50 (s, 1H), 8.29 (s, 1H), 8.03 (d, J = 5.7 Hz, 1H), 7.57 (t, J = 8.8 Hz, 1H), 7.34 (s, 1H), 7.22 (dd, J = 11.0, 2.6 Hz, 1H), 7.10 (s, 1H), 7.05 (dd, J = 8.6, 1.9 Hz, 1H), 6.87 (dd, J = 16.7, 10.5 Hz, 1H), 6.22 (dd, J = 5.7, 2.0 Hz, 1H), 6.15-6.05 (m, 2H), 5.69 (dd, J = 10.4, 2.4 Hz, 1H), 5.43 (d, J = 8.4 Hz, 1H), 4.47 (d, J = 13.2 Hz, 1H), 4.14 (d, J = 12.9 Hz, 1H), 3.98-3.95 (m, 5H), 3.81-3.74 (m, 1H), 3.51-3.35 (m, 9H), 3.27-3.15 (m, 2H), 2.86 (t, J = 12.3 Hz, 1H), 2.11-2.02 (m, 2H), 1.49- 1.38 (m, 2H). |
| 256 | 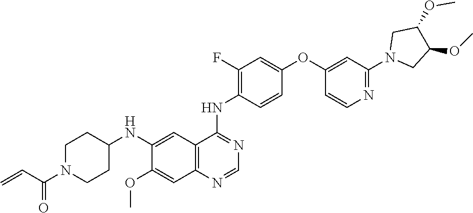 [SMILES: C=CC(=O)N1CCC(Nc2cc3c(Nc4ccc(Oc5ccnc(N6C[C@H](OC)[C@@H](OC)C6)c5)cc4F)ncnc3cc2OC)CC1] | LC/MS: 644.1 [M + H]⁺. ¹H NMR (400 MHz,) δ 9.66(S, 1H), 8.31 (s, 1H), 7.99 (d, J = 5.6 Hz, 1H), 7.54 (t, J = 8.8 Hz, 1H), 7.32 (s, 1H), 7.20 (dd, J = 11.2, 2.7 Hz, 1H), 7.07 (s, 1H), 7.02 (ddd, J = 8.8, 2.4, 0.8 Hz, 1H), 6.82 (dd, J = 16.8, 10.4 Hz, 1H), 6.18 (dd, J = 5.6, 2.0 Hz, 1H), 6.08 (dd, J = 16.8, 2.4 Hz, 1H), 6.04 (d, J = 2.0 Hz, 1H), 5.65 (dd, J = 10.4, 2.4 Hz, 1H), 5.47 (d, J = 8.4 Hz, 1H), 4.47- 4.38 (m, 1H), 4.15-4.05 (m, 1H), 3.94 (s, 3H), 3.92-3.90 (m, 2H), 3.74 (dd, J = 8.4, 4.0 Hz, 1H), 3.44-3.38 (m, 4H), 3.28 (s, 6H), 3.22- 3.17 (m, 1H), 2.86-2.76 (m, 1H), 2.12-2.03 (m, 2H), 1.46-1.35 (m, 2H). |
| 257 | 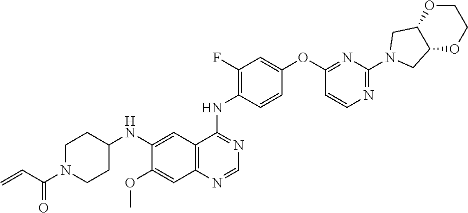 [SMILES: C=CC(=O)N1CCC(Nc2cc3c(Nc4ccc(Oc5ccnc(N6C[C@@H]7OCCO[C@@H]7C6)n5)cc4F)ncnc3cc2OC)CC1] | LC/MS: 643.1 [M + H]⁺. ¹H NMR (400 MHz, DMSO) δ 9.21 (s, 1H), 8.29 (d, J = 5.6 Hz, 1H), 8.22 (s, 1H), 7.58 (t, J = 8.7 Hz, 1H), 7.39-7.27 (m, 2H), 7.14-7.09 (m, 2H), 6.87 (dd, J = 16.4, 10.4 Hz, 1H), 6.27 (d, J = 5.6 Hz, 1H), 6.12 (d, J = 16.4 Hz, 1H), 5.69 (d, J = 10.4 Hz, 1H), 5.34 (d, J = 8.4 Hz, 1H), 4.47 (d, J = 12.4 Hz, 1H), 4.23-4.14 (m, 3H), 3.97 (s, 3H), 3.82-3.73 (m, 3H), 3.69-3.48 (m, 4H), 3.42-3.34 (m, 2H), 3.23 (t, J = 12.4 Hz, 1H), 2.86 (t, J = 12.4 Hz, 1H), 2.24-2.03 (m, 2H), 1.52-1.34 (m, 2H). |
| 258 | 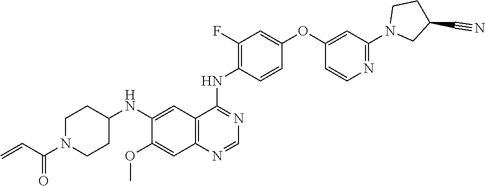 [SMILES: C=CC(=O)N1CCC(Nc2cc3c(Nc4ccc(Oc5ccnc(N6CC[C@@H](C#N)C6)c5)cc4F)ncnc3cc2OC)CC1] | LC/MS: 609.3 [M + H]⁺. ¹H NMR (400 MHz, DMSO) δ 9.21 (s, 1H), 8.23 (s, 1H), 8.06 (d, J = 5.7 Hz, 1H), 7.58 (t, J = 8.8 Hz, 1H), 7.30-7.18 (m, 2H), 7.11-7.00 (m, 2H), 6.86 (dd, J = 16.7, 10.5 Hz, 1H), 6.27 (dd, J = 5.7, 2.1 Hz, 1H), 6.17-6.07 (m, 2H), 5.69 (dd, J = 10.4, 2.5 Hz, 1H), 5.35 (d, J = 8.5 Hz, 1H), 4.46 (d, J = 12.8 Hz, 1H), 4.19-4.09 (m, 1H), 3.96 (s, 3H), 3.76-3.61 (m, 3H), 3.55- 3.38 (m, 3H), 3.27-3.18 (m, 1H), 2.85 (t, J = 12.2 Hz, 1H), 2.34 (d, J = 6.9 Hz, 1H), 2.32- 2.23 (m, 1H), 2.14-2.05 (m, 2H), 1.52-1.46 |
| | | (m, 2H). |
| 259 | 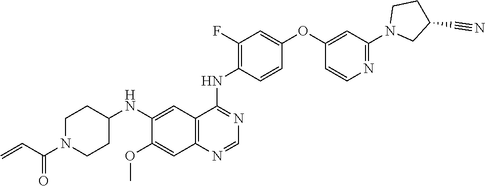 [SMILES: C=CC(=O)N1CCC(Nc2cc3c(Nc4ccc(Oc5ccnc(N6CC[C@H](C#N)C6)c5)cc4F)ncnc3cc2OC)CC1] | LC/MS: 609.3 [M + H]⁺. ¹H NMR (400 MHz, DMSO) δ 9.23 (s, 1H), 8.24 (s, 1H), 8.06 (d, J = 5.7 Hz, 1H), 7.58 (t, J = 8.8 Hz, 1H), 7.32-7.20 (m, 2H), 7.11-7.01 (m, 2H), 6.87 (dd, J = 16.7, 10.5 Hz, 1H), 6.27 (dd, J = 5.7, 2.0 Hz, 1H), 6.17-6.04 (m, 2H), 5.69 (dd, J = 10.4, 2.4 Hz, 1H), 5.36 (d, J = 8.6 Hz, 1H), 4.46 (d, J = 12.5 Hz, 1H), 4.14 (d, J = 13.7 Hz, 1H), 3.97 (s, 3H), 3.79-3.39 (m, 6H), 3.27- 3.13 (m, 1H), 2.85 (t, J = 11.9 Hz, 1H), 2.40- 2.29 (m, 1H), 2.29-2.17 (m, 1H), 2.14-2.03 (m, 2H), 1.50-1.38 (m, 2H). |
| 261 | 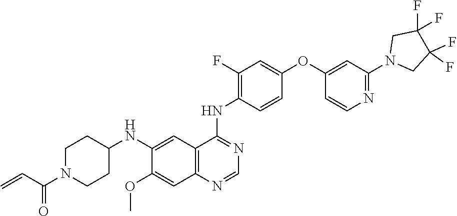 [SMILES: C=CC(=O)N1CCC(Nc2cc3c(Nc4ccc(Oc5ccnc(N6CC(F)(F)C(F)(F)C6)c5)cc4F)ncnc3cc2OC)CC1] | LC/MS: 655.8 [M + H]⁺. ¹H NMR (400 MHz, DMSO) δ 9.19 (s, 1H), 8.26- 8.08 (m, 2H), 7.59 (t, J = 8.8 Hz, 1H), 7.27- 6.82 (m, 5H), 6.41-6.09 (m, 3H), 5.68 (dd, J = 10.4, 2.5 Hz, 1H), 5.33 (d, J = 8.6 Hz, 1H), 4.53- 4.41 (m, 1H), 4.18-4.10 (m, 4H), 3.96 (s, 3H), 3.82-3.69 (m, 1H), 3.29-3.19 (m, 1H), 2.91- 2.80 (m, 1H), 2.56 (s, 1H), 2.13-2.01 (m, 2H), 1.51-1.35 (m, 2H). |
| 262 | 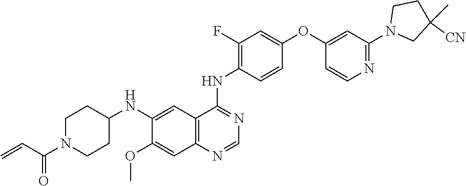 [SMILES: C=CC(=O)N1CCC(Nc2cc3c(Nc4ccc(Oc5ccnc(N6CCC(C)(C#N)C6)c5)cc4F)ncnc3cc2OC)CC1] | LC/MS: 623.3 [M + H]⁺. ¹H NMR (400 MHz, DMSO) δ 9.18 (s, 1H), 8.23 (s, 1H), 8.05 (d, J = 5.7 Hz, 1H), 7.58 (t, J = 8.8 Hz, 1H), 7.27-6.84 (m, 5H), 6.29-6.08 (m, 3H), 5.68 (dd, J = 10.4, 2.5 Hz, 1H), 5.33 (d, J = 8.5 Hz, 1H), 4.51-4.40 (m, 1H), 4.18-4.08 (m, 1H), 3.96 (s, 3H), 3.82-3.69 (m, 1H), 3.57- 3.44 (m, 2H), 3.28-3.18 (m, 1H), 2.94-2.80 (m, 1H), 2.57 (s, 2H), 2.47-2.39 (m, 1H), 2.16- 2.03 (m, 3H), 1.53-1.39 (m, 5H). |
| 263 | 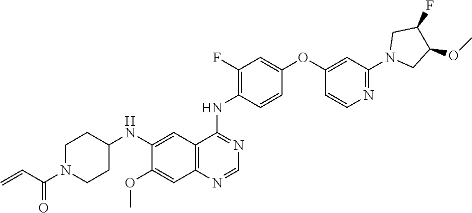 [SMILES: C=CC(=O)N1CCC(Nc2cc3c(Nc4ccc(Oc5ccnc(N6C[C@@H](F)[C@@H](OC)C6)c5)cc4F)ncnc3cc2OC)CC1] | LC/MS: 632.1 [M + H]⁺. ¹H NMR (400 MHz, DMSO) δ 9.18 (s, 1H), 8.22 (s, 1H), 8.04 (d, J = 5.7 Hz, 1H), 7.57 (t, J = 8.8 Hz, 1H), 7.25 (s, 1H), 7.21 (dd, J = 10.9, 2.6 Hz, 1H), 7.08 (s, 1H), 7.05-7.00 (m, 1H), 6.87 (dd, J = 16.7, 10.5 Hz, 1H), 6.25 (dd, J = 5.7, 2.1 Hz, 1H), 6.10 (dd, J = 10.3, 8.0 Hz, 2H), 5.69 (dd, J = 10.4, 2.5 Hz, 1H), 5.38-5.27 (m, 2H), 4.51- 4.43 (m, 1H), 4.16-4.07 (m, 2H), 3.96 (s, 3H), 3.83-3.57 (m, 5H), 3.39 (s, 3H), 3.23-3.15 (m, 2H), 2.84 (d, J = 11.8 Hz, 1H), 2.18-2.04 (m, 2H), 1.53-1.32 (m, 2H). |
| 264 | 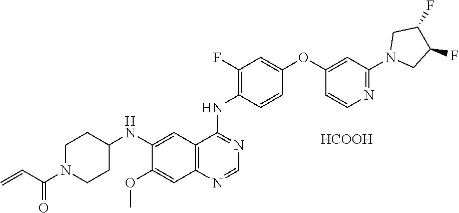 [SMILES: C=CC(=O)N1CCC(Nc2cc3c(Nc4ccc(Oc5ccnc(N6C[C@H](F)[C@@H](F)C6)c5)cc4F)ncnc3cc2OC)CC1 ; "HCOOH"] | LC/MS: 620.1 [M + H]⁺. 1H NMR (400 MHz, DMSO-d6) δ 9.19 (s, 1H), 8.22 (s, 1H), 8.07 (d, J = 5.7 Hz, 1H), 7.58 (t, J = 8.8 Hz, 1H), 7.26 (s, 1H), 7.23 (dd, J = 11.0, 2.7 Hz, 1H), 7.08 (s, 1H), 7.05 (dd, J = 8.6, 1.9 Hz, 1H), 6.87 (dd, J = 16.7, 10.5 Hz, 1H), 6.28 (dd, J = 5.7, 2.1 Hz, 1H), 6.18 (d, J = 2.0 Hz, 1H), 6.12 (dd, J = 16.7, 2.5 Hz, 1H), 5.69 (dd, J = 10.4, 2.5 Hz, 1H), 5.56-5.49 (m, 1H), 5.44- 5.38 (m, 1H), 5.34 (d, J = 8.5 Hz, 1H), 4.46 (d, J = 13.0 Hz, 1H), 4.14 (d, J = 13.6 Hz, 1H), 3.96 (s, 3H), 3.86-3.59 (m, 6H), 3.25-3.16 (m, 2H), 2.86 (t, J = 12.2 Hz, 1H), 2.13-2.02 (m, 2H), 1.51-1.36 (m, 2H). |
| 265 | 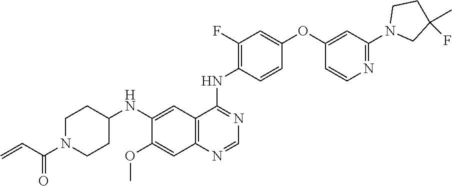 [SMILES: C=CC(=O)N1CCC(Nc2cc3c(Nc4ccc(Oc5ccnc(N6CCC(C)(F)C6)c5)cc4F)ncnc3cc2OC)CC1] | LC/MS: 616.3 [M + H]⁺. 1H NMR (400 MHz, DMSO-d6) δ 9.17 (s, 1H), 8.22 (s, 1H), 8.03 (d, J = 5.7 Hz, 1H), 7.58 (t, J = 8.8 Hz, 1H), 7.26 (s, 1H), 7.21 (dd, J = 11.0, 2.7 Hz, 1H), 7.08 (s, 1H), 7.04 (dd, J = 8.6, 1.9 Hz, 1H), 6.86 (dd, J = 16.7, 10.5 Hz, 1H), 6.23 (dd, J = 5.7, 2.1 Hz, 1H), 6.12 (dd, J = 16.7, 2.5 Hz, 1H), 6.03 (d, J = 2.0 Hz, 1H), 5.68 (dd, J = 10.4, 2.5 Hz, 1H), 5.33 (d, J = 8.6 Hz, 1H), 4.46 (d, J = 12.2 Hz, 1H), 4.13 (d, J = 11.7 Hz, 1H), 3.96 (s, 3H), 3.81-3.53 (m, 3H), 3.50-3.35 (m, 2H), 3.23 (t, J = 12.3 Hz, 1H), 2.86 (t, J = 11.5 |
| | | Hz, 1H), 2.27-2.00 (m, 4H), 1.56 (d, J = 20.9 |
| | | Hz, 3H), 1.50-1.36 (m, 2H). |
| 266 | 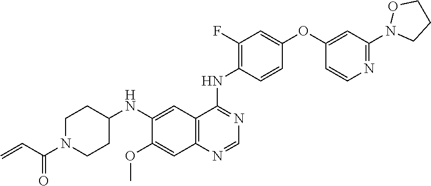 [SMILES: C=CC(=O)N1CCC(Nc2cc3c(Nc4ccc(Oc5ccnc(N6CCCO6)c5)cc4F)ncnc3cc2OC)CC1] | LC/MS: 586.2 [M + H]⁺. 1H NMR (400 MHz, DMSO) δ 10.82 (s, 1H), 8.74 (s, 1H), 8.20 (d, J = 5.7 Hz, 1H), 7.68 (t, J = 8.7 Hz, 1H), 7.49-7.43 (m, 2H), 7.24-7.17 (m, 2H), 6.87 (dd, J = 16.7, 10.5 Hz, 1H), 6.64 (dd, J = 5.7, 2.3 Hz, 1H), 6.58 (d, J = 2.2 Hz, 1H), 6.16-6.04 (m, 2H), 5.70 (dd, J = 10.4, 2.4 Hz, 1H), 4.49 (d, J = 13.2 Hz, 1H), 4.17 (d, J = 12.9 Hz, 1H), 4.05 (s, 3H), 3.90-3.74 (m, 5H), 3.27-3.15 (m, 1H), 2.84 (t, J = 11.6 Hz, 1H), 2.17 (dd, J = 14.5, 7.2 Hz, 2H), 2.05 (d, J = 12.5 Hz, 2H), 1.53-1.46 (m, 2H). |
| 267 | 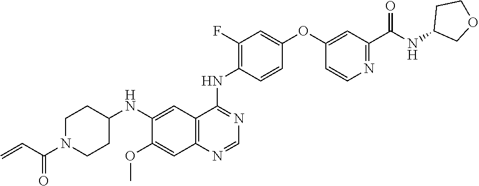 [SMILES: C=CC(=O)N1CCC(Nc2cc3c(Nc4ccc(Oc5ccnc(C(=O)N[C@@H]6CCOC6)c5)cc4F)ncnc3cc2OC)CC1] | LC/MS: 628.3 [M + H]⁺. 1H NMR (400 MHz, DMSO) δ 9.24 (s, 1H), 8.79 (d, J = 7.2 Hz, 1H), 8.59 (d, J = 5.6 Hz, 1H), 8.37 (s, 1H), 8.25 (s, 1H), 7.67 (t, J = 8.7 Hz, 1H), 7.47 (d, J = 2.5 Hz, 1H), 7.38 (dd, J = 10.7, 2.5 Hz, 1H), 7.33-7.21 (m, 2H), 7.16 (dd, J = 8.7 Hz, 1H), 7.09 (s, 1H), 6.86 (dd, J = 16.7, 10.5 Hz, 1H), 6.12 (dd, J = 16.7, 2.4 Hz, 1H), 5.68 (dd, J = 10.4, 2.4 Hz, 1H), 5.33 (d, J = 8.5 Hz, 1H), 4.46 (d, J = 6.2 Hz, 2H), 4.13 (d, J = 13.9 Hz, 1H), 3.97 (s, 3H), 3.88-3.80 (m, 2H), 3.78-3.71 (m, 2H), 3.60 (dd, J = 8.8, 4.7 Hz, |
| | | 1H), 3.28-3.20 (m, 1H), 2.92-2.84 (m, 1H), |
| | | 2.18-2.05 (m, 3H), 2.01-1.94 (m, 1H), 1.51- |
| | | 1.42 (m, 2H). |
| 268 | 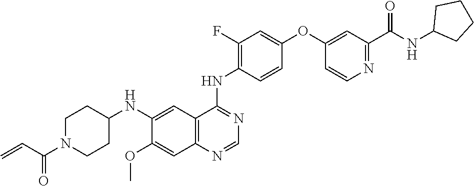 [SMILES: C=CC(=O)N1CCC(Nc2cc3c(Nc4ccc(Oc5ccnc(C(=O)NC6CCCC6)c5)cc4F)ncnc3cc2OC)CC1] | LC/MS: 626.2 [M + H]⁺. 1H NMR (400 MHz, DMSO) δ 10.83 (s, 1H), 8.75 (s, 1H), 8.60 (t, J = 6.9 Hz, 2H), 7.72 (t, J = 8.7 Hz, 1H), 7.52 (dd, J = 10.7, 2.6 Hz, 1H), 7.46 (d, J = 2.4 Hz, 2H), 7.32 (dd, J = 5.6, 2.6 Hz, 1H), 7.27 (dd, J = 8.6, 1.8 Hz, 1H), 7.19 (s, 1H), 6.87 (dd, J = 16.7, 10.5 Hz, 1H), 6.13 (dd, J = 16.7, 2.4 Hz, 1H), 6.07-6.02 (m, 1H), 5.70 (dd, J = 10.4, 2.4 Hz, 1H), 4.49 (d, J = 11.7 Hz, 1H), 4.24-4.09 (m, 2H), 4.05 (s, 3H), 3.77 (d, 1H), 3.24 (t, J = 11.4 Hz, 1H), 2.85 (t, J = 11.0 Hz, 1H), 2.06 (t, J = 14.7 Hz, 2H), 1.93-1.84 |
| | | (m, 2H), 1.75-1.67 (m, 1H), 1.65-1.44 (m, |
| | | 6H). |
| 269 | 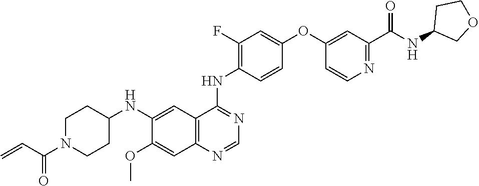 [SMILES: C=CC(=O)N1CCC(Nc2cc3c(Nc4ccc(Oc5ccnc(C(=O)N[C@H]6CCOC6)c5)cc4F)ncnc3cc2OC)CC1] | LC/MS: 628.3 [M + H]⁺. 1H NMR (400 MHz, DMSO) δ 10.83 (s, 1H), 8.85 (d, J = 7.3 Hz, 1H), 8.76 (s, 1H), 8.62 (d, J = 5.6 Hz, 1H), 7.73 (t, J = 8.7 Hz, 1H), 7.53 (dd, J = 10.7, 2.6 Hz, 1H), 7.46 (d, J = 2.7 Hz, 2H), 7.34 (dd, J = 5.6, 2.6 Hz, 1H), 7.29-7.25 (m, 1H), 7.18 (s, 1H), 6.87 (dd, J = 16.7, 10.5 Hz, 1H), 6.15-6.04 (m, 2H), 5.70 (dd, J = 10.4, 2.4 Hz, 1H), 4.52-4.42 (m, 2H), 4.17 (d, J = 14.0 Hz, 1H), 4.05 (s, 3H), 3.90-3.75 (m, 3H), 3.74- 3.68 (m, 1H), 3.59 (dd, J = 8.8, 4.7 Hz, 1H), 3.22 (t, J = 12.6 Hz, 1H), 2.83 (t, J = 12.7 Hz, |
| | | 1H), 2.19-2.11 (m, 1H), 2.07-1.95 (m, 3H), |
| | | 1.55-1.44 (m, 2H). |
| 274 | 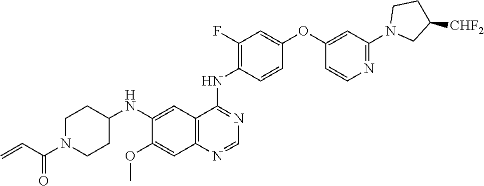 [SMILES: C=CC(=O)N1CCC(Nc2cc3c(Nc4ccc(Oc5ccnc(N6CC[C@@H](C(F)F)C6)c5)cc4F)ncnc3cc2OC)CC1] | LC/MS: 634.9 [M + H]⁺. 1H NMR (400 MHz, DMSO) δ 9.17 (s, 1H), 8.22 (s, 1H), 8.20 (s, 1H), 8.04 (d, J = 5.7 Hz, 1H), 7.58 (t, J = 8.8 Hz, 1H), 7.26 (s, 1H), 7.20 (dd, J = 11.0, 2.6 Hz, 1H), 7.08 (s, 1H), 7.05-7.01 (m, 1H), 6.89-6.82 (m, 1H), 6.31-6.00 (m, 4H), 5.68 (dd, J = 10.4, 2.4 Hz, 1H), 5.34-5.30 (m, 1H), 4.49-4.42 (m, 1H), 4.17-4.10 (m, 1H), 3.96 (s, 3H), 3.78-3.72 (m, 1H), 3.57- 3.51 (m, 1H), 3.49-3.45 (m, 1H), 3.38-3.34 (m, 2H), 3.26-3.19 (m, 1H), 2.90-2.80 (m, 2H), 2.14-2.05 (m, 3H), 2.01-1.93 (m, 1H), |
| | | 1.49-1.40 (m, 2H). |
| 275 | 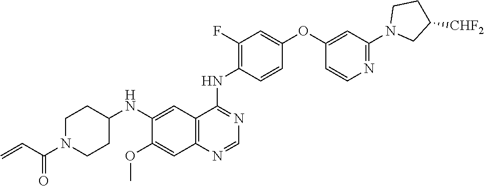 [SMILES: C=CC(=O)N1CCC(Nc2cc3c(Nc4ccc(Oc5ccnc(N6CC[C@H](C(F)F)C6)c5)cc4F)ncnc3cc2OC)CC1] | LC/MS: 634.2 [M + H]⁺. 1H NMR (400 MHz, DMSO) δ 9.19 (s, 1H), 8.22 (s, 1H), 8.15 (s, 1H), 8.04 (d, J = 6.0 Hz, 1H), 7.58 (t, J = 8.8 Hz, 1H), 7.27 (s, 1H), 7.18 (dd, J = 11.2, 2.8 Hz, 1H), 7.08 (s, 1H), 7.05-7.01 (m, 1H), 6.89-6.82 (m, 1H), 6.31-6.02 (m, 4H), 5.68 (dd, J = 10.4, 2.4 Hz, 1H), 5.34-5.30 (m, 1H), 4.49-4.42 (m, 1H), 4.17-4.10 (m, 1H), 3.96 (s, 3H), 3.78-3.72 (m, 1H), 3.57- 3.51 (m, 1H), 3.49-3.45 (m, 1H), 3.38-3.34 (m, 2H), 3.26-3.19 (m, 1H), 2.90-2.80 (m, 2H), 2.14-2.05 (m, 3H), 2.01-1.93 (m, 1H), |
| | | 1.49-1.40 (m, 2H). |
| 276 | 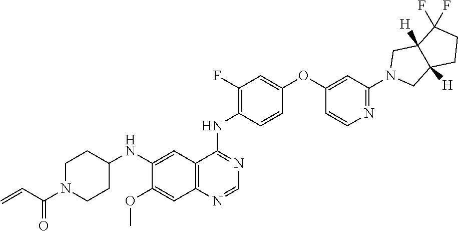 [SMILES: C=CC(=O)N1CCC(Nc2cc3c(Nc4ccc(Oc5ccnc(N6C[C@H]7CCC(F)(F)[C@H]7C6)c5)cc4F)ncnc3cc2OC)CC1] | LC/MS: 660.1 [M + H]⁺. 1H NMR (400 MHz, DMSO) δ 9.75 (brs, 1H), 8.36 (s, 1H), 8.05 (d, J = 5.6 Hz, 1H), 7.59 (t, J = 8.8 Hz, 1H), 7.38 (s, 1H), 7.24 (dd, J = 10.8, 2.4 Hz, 1H), 7.11 (s, 1H), 7.08-7.03 (m, 1H), 6.86 (dd, J = 16.8, 10.4 Hz, 1H), 6.27 (dd, J = 5.6, 2.0 Hz, 1H), 6.15-6.07 (m, 2H), 5.69 (dd, J = 10.4, 2.4 Hz, 1H), 5.51 (d, J = 8.4 Hz, 1H), 4.46 (d, J = 11.6 Hz, 1H), 4.13 (d, J = 13.2 Hz, 1H), 3.99 (s, 3H), 3.83-3.74 (m, 1H), 3.61 (d, J = 8.8 Hz, 1H), 3.55-3.50 (m, 1H), 3.49-3.43 (m, 1H), 3.28-3.20 (m, 2H), 3.04-2.99 (m, 2H), 2.86 (t, J = 12.0 Hz, 1H), 2.26-1.96 (m, 5H), 1.67-1.58 (m, 1H), 1.52-1.39 (m, 2H). |
| 282 | 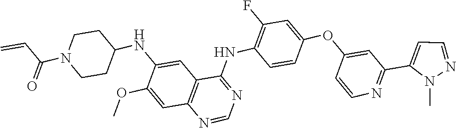 [SMILES: C=CC(=O)N1CCC(Nc2cc3c(Nc4ccc(Oc5ccnc(-c6ccnn6C)c5)cc4F)ncnc3cc2OC)CC1] | LC/MS: 595.2 [M + H]⁺. 1H NMR (400 MHz, DMSO) δ 9.26 (s, 1H), 8.62 (d, J = 5.7 Hz, 1H), 8.33 (s, 1H), 8.24 (s, 1H), 7.64 (t, J = 8.7 Hz, 1H), 7.47 (dd, J = 12.8, 2.1 Hz, 2H), 7.36 (dd, J = 10.8, 2.6 Hz, 1H), 7.28 (s, 1H), 7.15 (dd, J = 8.6, 1.9 Hz, 1H), 7.08 (s, 1H), 6.96 (dd, J = 5.7, 2.4 Hz, 1H), 6.87 (dd, J = 16.7, 10.5 Hz, 1H), 6.79 (d, J = 2.0 Hz, 1H), 6.12 (dd, J = 16.7, 2.4 Hz, 1H), 5.69 (dd, J = |
| | | 10.4, 2.5 Hz, 1H), 5.35 (d, J = 8.5 Hz, 1H), 4.46 |
| | | (d, J = 12.6 Hz, 1H), 4.20-4.10 (m, 4H), 3.97 |
| | | (s, 3H), 3.79-3.74 (m, 1H), 3.30-3.20 (m, 1H), |
| | | 2.90-2.80 (m, 1H), 2.15-2.00 (m, 2H), 1.50- |
| | | 1.37 (m, 2H). |
| 283 | 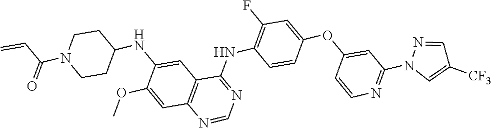 [SMILES: C=CC(=O)N1CCC(Nc2cc3c(Nc4ccc(Oc5ccnc(-n6cc(C(F)(F)F)cn6)c5)cc4F)ncnc3cc2OC)CC1] | LC/MS: 649.0 [M + H]⁺. 1H NMR (400 MHz, DMSO) δ 10.79 (s, 1H), 9.22 (s, 1H), 8.74 (s, 1H), 8.51 (d, J = 5.7 Hz, 1H), 8.29 (s, 1H), 7.75 (t, J = 8.7 Hz, 1H), 7.59 (dd, J = 10.6, 2.6 Hz, 1H), 7.46 (s, 1H), 7.38 (d, J = 2.3 Hz, 1H), 7.34 (dd, J = 8.7, 1.8 Hz, 1H), 7.22-7.16 (m, 2H), 6.88 (dd, J = 16.7, 10.5 Hz, 1H), 6.13 (dd, J = 16.7, 2.4 Hz, 1H), 6.05 (d, J = 8.3 Hz, 1H), 5.70 (dd, J = 10.4, 2.4 Hz, 1H), |
| | | 4.50 (d, J = 11.7 Hz, 1H), 4.17 (d, J = 13.3 Hz, |
| | | 1H), 4.05 (s, 3H), 3.79-3.74 (m, 1H), 3.23 (t, |
| | | J = 12.5 Hz, 1H), 2.85 (t, J = 12.3 Hz, 1H), 2.06 |
| | | (d, J = 11.4 Hz, 2H), 1.58-1.42 (m, 2H). |
| 289 | 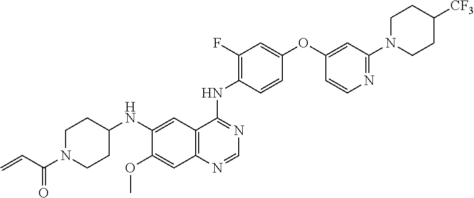 [SMILES: C=CC(=O)N1CCC(Nc2cc3c(Nc4ccc(Oc5ccnc(N6CCC(C(F)(F)F)CC6)c5)cc4F)ncnc3cc2OC)CC1] | LC/MS: 665.8 [M + H]⁺. 1H NMR (400 MHz, DMSO) δ 9.27 (brs, 1H), 8.25 (s, 1H), 8.13 (s, 1H), 8.07 (d, J = 5.7 Hz, 1H), 7.58 (t, J = 8.8 Hz, 1H), 7.27 (s, 1H), 7.22 (dd, J = 11.0, 2.7 Hz, 1H), 7.08 (s, 1H), 7.04 (dd, J = 8.7, 1.9 Hz, 1H), 6.90-6.83 (m, 1H), 6.49 (d, J = 1.9 Hz, 1H), 6.25 (dd, J = 5.7, 2.0 Hz, 1H), 6.12 (dd, J = 16.7, 2.5 Hz, 1H), 5.69 (dd, J = 10.4, 2.5 Hz, 1H), 5.37 (d, J = 8.4 Hz, 1H), 4.46 (d, J = 12.4 Hz, 1H), 4.37 (d, J = 13.0 Hz, 2H), 4.14 (d, J = 12.8 Hz, 1H), 3.97 (s, 3H), 3.78-3.72 (m, 1H), 3.21 (d, J = 12.6 Hz, 1H), 2.84 (t, J = 11.5 Hz, 2H), 2.69-2.57 (m, 1H), |
| | | 2.38-2.31 (m, 1H), 2.18-2.09 (m, 2H), 1.93- |
| | | 1.80 (m, 2H), 1.52-1.37 (m, 4H). |
| 293 | 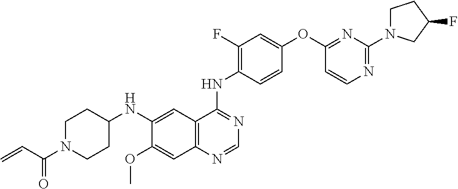 [SMILES: C=CC(=O)N1CCC(Nc2cc3c(Nc4ccc(Oc5ccnc(N6CC[C@@H](F)C6)n5)cc4F)ncnc3cc2OC)CC1] | LCMS: 603.1 [M + H]⁺. 1H NMR (400 MHz, DMSO) δ 9.19 (s, 1H), 8.29 (d, J = 5.6 Hz, 1H), 8.22 (s, 1H), 7.58 (t, J = 8.8 Hz, 1H), 7.32 (dd, J = 11.2, 2.4 Hz, 1H), 7.28 (s, 1H), 7.14 (dd, J = 8.8, 1.6 Hz, 1H), 7.08 (s, 1H), 6.86 (dd, J = 16.8, 10.4 Hz, 1H), 6.26 (d, J = 5.6 Hz, 1H), 6.12 (dd, J = 16.8, 2.4 Hz, 1H), 5.68 (dd, J = 10.4, 2.4 Hz, 1H), 5.48-5.30 (m, 2H), 4.46 (d, J = 14.0 Hz, 1H), 4.13 (d, J = 12.0 Hz, 1H), 3.96 (s, 3H), 3.82-3.70 (m, 2H), 3.61- 3.45 (m, 2H), 3.38-3.32 (m, 2H), 3.27-3.18 (m, 1H), 2.87 (t, J = 12.0 Hz, 1H), 2.23-2.15 |
| | | (m, 1H), 2.12-2.04 (m, 2H), 1.50-1.40 (m, |
| | | 2H). |
| 294 | 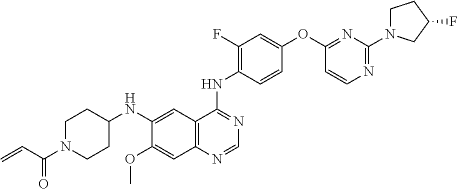 [SMILES: C=CC(=O)N1CCC(Nc2cc3c(Nc4ccc(Oc5ccnc(N6CC[C@H](F)C6)n5)cc4F)ncnc3cc2OC)CC1] | LCMS: 603.2 [M + H]⁺. 1H NMR (400 MHz, DMSO) δ 9.41 (s, 1H), 8.35- 8.25 (m, 2H), 7.58 (t, J = 8.8 Hz, 1H), 7.38- 7.30 (m, 2H), 7.15 (dd, J = 8.8, 2.0 Hz, 1H), 7.10 (s, 1H), 6.86 (dd, J = 16.8, 10.4 Hz, 1H), 6.27 (d, J = 5.6 Hz, 1H), 6.12 (dd, J = 16.8, 2.4 Hz, 1H), 5.68 (dd, J = 10.4, 2.4 Hz, 1H), 5.48- 5.29 (m, 2H), 4.46 (d, J = 13.2 Hz, 1H), 4.13 (d, J = 15.6 Hz, 1H), 3.97 (s, 3H), 3.83-3.67 (m, 2H), 3.65-3.47 (m, 2H), 3.42-3.33 (m, 2H), 3.24 (t, J = 11.2 Hz, 1H), 2.87 (t, J = 12.0 Hz, 1H), 2.24-2.16 (m, 1H), 2.13-2.06 (m, 2H), |
| | | 1.53-1.41 (m, 2H). |
| 295 | 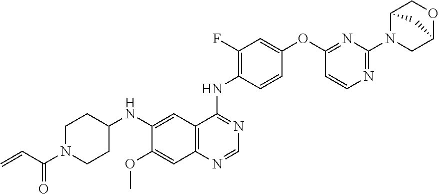 [SMILES: C=CC(=O)N1CCC(Nc2cc3c(Nc4ccc(Oc5ccnc(N6C[C@H]7C[C@@H]6CO7)n5)cc4F)ncnc3cc2OC)CC1] | LC/MS: 613.1 [M + H]⁺. 1H NMR (400 MHz, DMSO) δ 9.51 (s, 1H), 8.40- 8.17 (m, 2H), 7.57 (t, J = 8.8 Hz, 1H), 7.38- 6.80 (m, 5H), 6.29-6.05 (m, 2H), 5.75-5.41 (m, 2H), 4.65-4.43 (m, 2H), 4.19-3.61 (m, 7H), 3.51-3.39 (m, 1H), 3.30-3.07 (m, 3H), 2.91-2.79 (m, 1H), 2.12-1.78 (m, 4H), 1.54- 1.35 (m, 2H). |
| 296 | 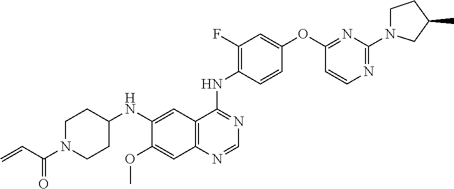 [SMILES: C=CC(=O)N1CCC(Nc2cc3c(Nc4ccc(Oc5ccnc(N6CC[C@@H](C)C6)n5)cc4F)ncnc3cc2OC)CC1] | LC/MS: 599.1 [M + H]⁺. 1H NMR (400 MHz, DMSO) δ 9.25 (brs, 1H), 8.33-8.11 (m, 3H), 7.57 (t, J = 8.8 Hz, 1H), 7.37-7.26 (m, 2H), 7.17-7.03 (m, 2H), 6.92- 6.81 (m, 1H), 6.23-6.07 (m, 2H), 5.69 (dd, J = 10.5, 2.4 Hz, 1H), 5.35 (d, J = 8.5 Hz, 1H), 4.52- 4.41 (m, 1H), 4.18-4.09 (m, 1H), 3.97 (s, 3H), 3.83-3.41 (m, 4H), 3.26-3.20 (m, 1H), 3.00- 2.73 (m, 2H), 2.33-2.20 (m, 1H), 2.13-1.97 (m, 3H), 1.56-1.38 (m, 3H), 1.03 (d, J = 4.8 Hz, 3H). |
| 297 | 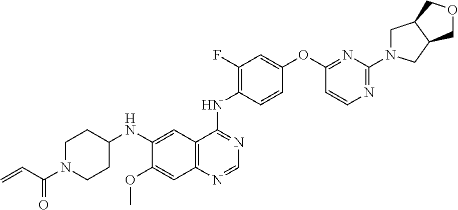 [SMILES: C=CC(=O)N1CCC(Nc2cc3c(Nc4ccc(Oc5ccnc(N6C[C@H]7COC[C@H]7C6)n5)cc4F)ncnc3cc2OC)CC1] | LC/MS: 627.3 [M + H]⁺. 1H NMR (400 MHz, DMSO) δ 9.21 (s, 1H), 8.34 (s, 2H), 8.27 (d, J = 5.6 Hz, 1H), 8.21 (s, 1H), 7.57 (t, J = 8.8 Hz, 1H), 7.33-7.28 (m, 2H), 7.13 (d, J = 8.4 Hz, 1H), 7.08 (s, 1H), 6.86 (dd, J = 16.8, 10.4 Hz, 1H), 6.22 (d, J = 5.4 Hz, 1H), 6.11 (dd, J = 16.8, 2.4 Hz, 1H), 5.68 (dd, J = 10.4, 2.4 Hz, 1H), 5.31 (d, J = 8.8 Hz, 1H), 4.48- 4.42 (m, 1H), 4.16-4.10 (m, 1H), 3.96 (s, 3H), 3.85-3.72 (m, 4H), 3.63-3.55 (m, 2H), 3.52 (d, J = 6.4 Hz, 2H), 3.24-3.18 (m, 2H), 2.98- 2.93(m, 2H), 2.89-2.82 (m, 1H), 2.11-2.04 (m, 2H), 1.47-1.39 (m, 2H). |
| 298 | 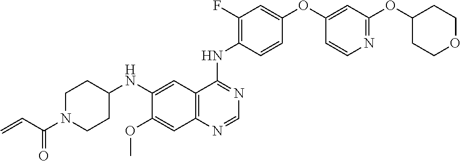 [SMILES: C=CC(=O)N1CCC(Nc2cc3c(Nc4ccc(Oc5ccnc(OC6CCOCC6)c5)cc4F)ncnc3cc2OC)CC1] | LC/MS: 615.3 [M + H]⁺. 1H NMR (400 MHz, DMSO) δ 9.33 (s, 1H), 8.27 (s, 1H), 8.11 (d, J = 5.6 Hz, 1H), 7.62 (t, J = 8.8 Hz, 1H), 7.35-7.27 (m, 2H), 7.13-7.07 (m, 2H), 6.86 (dd, J = 16.8, 10.4 Hz, 1H), 6.67 (dd, J = 5.6, 2.0 Hz, 1H), 6.22 (d, J = 2.0 Hz, 1H), 6.12 (dd, J = 16.6, 2.4 Hz, 1H), 5.69 (dd, J = 10.4, 2.4 Hz, 1H), 5.38 (d, J = 8.4 Hz, 1H), 5.24- 5.15 (m, 1H), 4.46 (d, J = 12.4 Hz, 1H), 4.14 (d, J = 13.2 Hz, 1H), 3.97 (s, 3H), 3.88-3.81 (m, 2H), 3.80-3.71 (m, 1H), 3.53-3.44 (m, |
| | | 2H), 3.23 (t, J = 12.4 Hz, 1H), 2.86 (t, J = 11.6 |
| | | Hz, 1H), 2.12-2.03 (m, 2H), 2.02-1.94 (m, |
| | | 2H), 1.68-1.56 (m, 2H), 1.49-1.37 (m, 2H). |
| 340 | 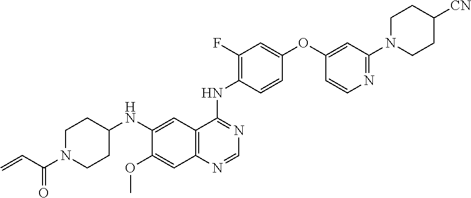 [SMILES: C=CC(=O)N1CCC(Nc2cc3c(Nc4ccc(Oc5ccnc(N6CCC(C#N)CC6)c5)cc4F)ncnc3cc2OC)CC1] | LCMS: 623.1 [M + H]⁺. 1H NMR (400 MHz, DMSO) δ 9.25 (s, 1H), 8.24 (s, 1H), 8.07 (d, J = 5.6 Hz, 1H), 7.58 (t, J = 8.8 Hz, 1H), 7.27 (s, 1H), 7.21 (dd, J = 10.8, 2.8 Hz, 1H), 7.08 (s, 1H), 7.03 (dd, J = 8.4, 2.0 Hz, 1H), 6.86 (dd, J = 16.8, 10.4 Hz, 1H), 6.48 (d, J = 2.0 Hz, 1H), 6.26 (dd, J = 5.6, 2.0 Hz, 1H), 6.12 (dd, J = 16.8, 2.4 Hz, 1H), 5.68 (dd, J = 10.4, 2.4 Hz, 1H), 5.35 (d, J = 8.4 Hz, 1H), 4.46 (d, J = 13.2 Hz, 1H), 4.13 (d, J = 13.2 Hz, 1H), 3.97 (s, 3H), 3.84-3.70 (m, 3H), 3.38-3.33 (m, 2H), 3.23 (t, J = 14.0 Hz, 1H), 3.15-3.07 (m, 1H), 2.86 (t, J = 12.0 Hz, 1H), 2.12-2.03 (m, 2H), 1.97-1.86 |
| | | (m, 2H), 1.78-1.66 (m, 2H), 1.52-1.37 (m, |
| | | 2H). |

### Testing of Compounds for Activity

### Assays

### 1. Preparation of HCC827-SVD, HCC827-ASV and HCC827-YVMA Cell Lines

HCC827-SVD cell line were generated by first transducing HCC827 cells (ATCC) with lentiviral particles expressing codon-optimized human EGFR exon 20 insertion mutant D770_N771 insSVD, and then selecting under 5 μg/ml purimycin treatment for a week.

The resulting cells were further transduced with lentiviral particles carrying EGFR sgRNA and CAS9 to knockout the endogenous alleles of EGFR, and then selected under 500 μg/ml hygromycin B treatment for two weeks. Single clone was subsequently generated by limiting dilution approach. Endogenous EGFR knockout was confirmed by Sanger sequencing of cell genomic DNA and expression of exogenous mutant EGFR was verified by Western blot analysis.

HCC827-ASV and HCC827-YVMA cell lines were generated in the similar approach as HCC827-SVD except that HCC827 cells (ATCC) were first transduced with lentiviral particles expressing codon-optimized human EGFR exon 20 insertion mutant A769_D770insASV and human ERBB2 exon20 insertion mutant A775_G776ins YVMA, respectively.

### 2. Cell Growth Inhibition for HCC827, HCC827-SVD, HCC827-ASV, HCC827-YVMA and A431.

HCC827(ATCC), HCC827-SVD, HCC827-ASV, HCC827-YVMA were seeded in 96-well plates at 5000 cells/well in 90 μl of RPMI growth medium containing 10% FBS and 1% Penicillin Streptomycin. A431 (ATCC) cells were seed in 96-well plates at 5000 cells/well in 90 μl of DMEM growth medium containing 10% FBS and 1% Penicillin Streptomycin. Cells were incubated at 3700 overnight. The following day, the test compound was administered to the cells by using 1 Ox compound stock solution prepared in growth medium at various concentrations. After administration of the compound, cells were then incubated at 3700 for 3 days. Before CeIlTiter-Glo assay, the plates were equilibrated at room temperature for approximately 10 minutes. 100 ul of CelTiter-Glo® Reagent (Promega) was added to each well. The plates were then incubated at room temperature for 10 minutes and luminescence was recorded by EnSpire plate reader (PerkinElmer).

### Results

Table 3 illustrates growth inhibition (G150) by exemplary compounds of the present disclosure in multiple cell lines 3 days after administration.

TABLE 3
Cell Growth Inhibition of the Compounds of the
Present Disclosure

| | GI50 (nM). A <30; B ≥30 and <60; C ≥60 | | | | |
|---|---|---|---|---|---|
| | and <100; D ≥100 and <300; E ≥300 and <600; | | | | |
| | F ≥600 and <1000; G ≥1000 | | | | |
| Compound # | | HCC827- | HCC827- | HCC827- | |
| in Patent | HCC827 | SVD | ASV | YVMA | A431 |
| 1 | E | C | B | C | E |
| 2 | A | A | A | A | C |
| 3 | E | C | B | B | D |
| 4 | E | D | D | D | ES |
| 5 | D | D | D | D | E |
| 6 | G | E | E | E | E |
| 7 | A | A | A | A | A |
| 8 | D | B | D | B | E |
| 9 | G | G | G | G | G |
| 10 | B | A | A | A | E |
| 11 | C | B | B | C | D |
| 12 | B | A | A | A | D |
| 13 | A | A | A | A | E |
| 14 | C | D | D | C | C |
| 15 | D | D | D | C | D |
| 16 | A | A | A | A | E |
| 17 | C | D | F | F | D |
| 18 | A | A | A | A | D |
| 19 | A | B | B | C | D |
| 20 | B | D | C | D | D |
| 21 | G | G | G | E | G |
| 23 | G | G | G | F | G |
| 24 | G | G | G | G | G |
| 25 | G | G | G | G | G |
| 26 | C | B | B | B | D |
| 27 | D | C | B | C | G |
| 28 | D | B | A | B | D |
| 29 | A | A | A | A | G |
| 30 | D | B | A | A | E |
| 31 | A | B | B | A | A |
| 32 | C | A | A | A | D |
| 33 | E | D | C | D | D |
| 34 | F | B | C | C | G |
| 35 | D | A | A | A | G |
| 36 | C | B | C | C | B |
| 37 | B | A | A | A | E |
| 38 | D | B | B | B | B |
| 39 | C | A | A | A | E |
| 41 | C | A | A | A | E |
| 42 | G | F | E | F | G |
| 43 | C | A | A | A | E |
| 44 | B | B | B | A | D |
| 45 | D | C | A | C | E |
| 46 | D | B | C | A | G |
| 47 | B | B | A | A | E |
| 48 | C | B | B | B | C |
| 50 | C | B | B | B | D |
| 51 | B | B | B | B | D |
| 53 | B | A | A | A | D |
| 54 | A | A | A | A | D |
| 55 | A | A | A | A | D |
| 56 | B | A | A | A | A |
| 58 | C | C | C | C | E |
| 59 | C | C | B | B | D |
| 79 | G | G | G | G | G |
| 80 | G | G | G | G | G |
| 81 | E | D | N/A | N/A | E |
| 84 | G | G | G | G | G |
| 86 | B | B | C | B | C |
| 87 | D | B | C | B | D |
| 88 | B | D | C | B | G |
| 89 | B | A | A | A | F |
| 90 | C | C | D | C | D |
| 91 | D | B | C | D | D |
| 92 | C | A | A | A | D |
| 93 | C | C | B | B | G |
| 94 | B | D | B | B | G |
| 95 | A | A | A | A | D |
| 96 | C | D | D | D | E |
| 97 | B | A | A | A | E |
| 98 | C | A | A | A | D |
| 99 | C | C | B | B | F |
| 100 | D | D | D | D | E |
| 101 | C | A | A | A | G |
| 102 | D | C | D | D | D |
| 103 | B | A | A | A | D |
| 104 | D | A | A | A | G |
| 105 | E | A | A | A | G |
| 106 | D | A | A | A | G |
| 107 | D | F | E | E | E |
| 108 | E | E | D | D | G |
| 109 | A | G | G | G | G |
| 110 | A | G | G | G | D |
| 111 | B | E | E | E | F |
| 112 | G | D | E | F | G |
| 113 | B | A | A | A | F |
| 114 | E | D | C | D | G |
| 115 | E | C | D | D | E |
| 116 | E | D | D | C | F |
| 117 | E | D | D | C | G |
| 118 | E | B | B | B | G |
| 119 | E | D | C | D | D |
| 120 | G | D | D | F | G |
| 121 | E | A | A | A | G |
| 122 | F | F | F | G | G |
| 123 | G | E | E | F | G |
| 124 | F | B | B | C | G |
| 125 | G | E | D | D | G |
| 126 | G | D | C | D | G |
| 127 | F | D | D | D | G |
| 128 | E | C | D | D | G |
| 129 | E | C | B | C | G |
| 130 | D | B | B | B | E |
| 131 | F | C | B | C | G |
| 132 | D | A | A | A | F |
| 133 | D | A | A | A | F |
| 134 | D | B | A | B | F |
| 135 | E | C | A | B | G |
| 136 | E | B | B | B | F |
| 137 | G | D | D | E | G |
| 138 | D | A | A | A | G |
| 139 | D | D | C | D | E |
| 140 | F | D | D | E | F |
| 141 | D | B | A | B | G |
| 142 | D | A | A | A | G |
| 143 | G | E | E | E | G |
| 144 | G | F | F | G | G |
| 145 | G | G | G | G | G |
| 146 | F | C | C | D | G |
| 147 | D | C | C | C | G |
| 148 | D | B | B | B | E |
| 149 | D | B | B | C | D |
| 150 | A | A | A | A | D |
| 151 | D | A | A | A | F |
| 152 | F | C | A | C | G |
| 153 | A | A | A | B | E |
| 154 | G | E | D | E | G |
| 155 | G | D | D | D | G |
| 156 | G | D | D | D | G |
| 157 | G | G | G | G | G |
| 158 | F | C | D | E | G |
| 159 | D | B | B | C | E |
| 160 | B | A | A | A | C |
| 161 | D | A | A | A | E |
| 162 | D | D | D | D | G |
| 163 | G | B | B | D | G |
| 164 | E | A | A | A | F |
| 165 | E | A | A | B | G |
| 166 | E | G | D | E | G |
| 167 | A | C | B | C | F |
| 168 | G | E | G | G | G |
| 169 | G | G | G | G | G |
| 170 | F | C | C | D | G |
| 171 | E | B | B | B | F |
| 172 | E | A | A | A | F |
| 173 | E | D | C | D | G |
| 174 | F | D | D | D | G |
| 175 | F | B | B | B | G |
| 176 | D | B | A | A | F |
| 177 | D | A | A | A | E |
| 178 | G | E | E | E | G |
| 179 | G | C | A | B | G |
| 180 | G | B | B | B | G |
| 181 | A | A | A | A | D |
| 182 | F | F | F | E | G |
| 183 | G | G | G | G | G |
| 184 | G | G | G | G | G |
| 185 | A | A | A | A | D |
| 186 | F | G | G | G | G |
| 187 | G | D | D | E | G |
| 188 | C | A | A | B | D |
| 189 | E | A | A | A | G |
| 190 | B | A | A | A | E |
| 191 | B | A | A | A | E |
| 192 | C | A | A | A | D |
| 193 | D | A | A | A | G |
| 194 | C | A | A | A | E |
| 195 | D | A | A | A | F |
| 196 | C | A | A | A | D |
| 197 | C | A | A | A | E |
| 198 | B | A | A | A | F |
| 199 | D | A | A | A | C |
| 200 | C | B | B | C | G |
| 201 | C | A | A | A | F |
| 202 | A | A | A | A | D |
| 203 | D | A | A | A | D |
| 204 | C | A | A | A | D |
| 205 | A | A | A | A | D |
| 206 | C | A | A | A | D |
| 207 | B | A | A | A | D |
| 208 | D | A | A | B | F |
| 209 | D | D | D | C | F |
| 210 | E | C | B | A | F |
| 211 | G | G | G | F | G |
| 212 | G | G | G | G | G |
| 213 | E | E | D | E | G |
| 214 | E | B | A | A | G |
| 217 | G | E | F | F | G |
| 218 | D | B | B | B | G |
| 219 | D | A | A | A | E |
| 220 | D | A | A | A | F |
| 221 | D | A | A | A | D |
| 222 | C | A | A | A | F |
| 223 | E | D | D | D | F |
| 224 | G | E | F | F | G |
| 225 | G | E | E | E | G |
| 226 | G | E | G | G | G |
| 227 | D | B | C | C | E |
| 228 | E | D | C | C | G |
| 229 | D | C | D | D | E |
| 230 | E | B | A | A | G |
| 231 | G | E | D | D | G |
| 232 | C | B | A | A | G |
| 233 | C | B | A | A | G |
| 234 | C | B | A | A | E |
| 235 | C | B | B | C | F |
| 236 | D | A | A | A | G |
| 237 | E | B | C | A | G |
| 238 | D | A | A | A | G |
| 239 | A | A | C | C | E |
| 240 | F | E | E | E | G |
| 241 | A | B | C | C | F |
| 242 | G | D | D | D | G |
| 243 | B | A | A | A | E |
| 244 | A | A | A | A | C |
| 245 | D | B | A | A | E |
| 246 | B | A | A | A | D |
| 247 | D | C | C | B | F |
| 248 | C | B | B | B | E |
| 249 | D | B | C | C | F |
| 250 | D | A | A | A | G |
| 251 | D | A | B | B | E |
| 252 | D | B | B | B | F |
| 253 | D | B | B | B | E |
| 254 | C | A | A | A | G |
| 255 | E | A | A | A | G |
| 256 | C | A | A | A | D |
| 257 | C | A | A | A | F |
| 258 | C | A | A | A | F |
| 259 | C | A | A | A | G |
| 260 | D | A | A | A | G |
| 261 | D | B | B | B | F |
| 262 | B | A | B | B | F |
| 263 | D | A | A | A | F |
| 264 | C | A | A | A | F |
| 265 | B | A | A | A | D |

1. A compound of Formula (I) or a tautomer, stereoisomer or a mixture of stereoisomers, or a pharmaceutically acceptable salt, or hydrate, or deuterated derivative thereof: 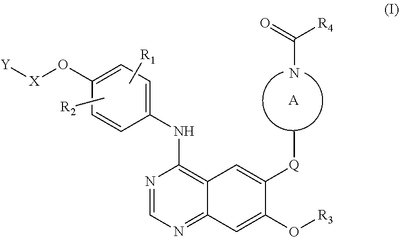
wherein:
X is absent or is chosen from 5- to 6-member cycloalkyl, 5- to 6-member aryl, 5- to 6-member heterocycle, or a monocyclic or bicyclic 5- to 12-member heteroaryl, each of which is independently substituted with 0, 1, 2, or 3 R^{a};
Y is absent or is chosen from an ester group, a carboxylic acid group, an amide group, an amino group, an alkylamino group, a dialkylamino group, C₁₋₄ alkyl, O-heterocycle, NH-heterocycle, 5- to 6-member cycloalkyl, 5- to 6-member aryl, 4- to 10-member monocyclic or bicyclic heterocycle, or a monocyclic or bicyclic 5- to 12-member heteroaryl, each of which is independently substituted with 0, 1, 2, or 3 R^{a};
R₁ and R₂ are each independently chosen from hydrogen, halogen, —CN, hydroxyl, C₁-C₅alkyl, deuterated C₁-C₅alkyl, C₁-C₅alkoxy, deuterated C₁-C₅alkoxy, and C₁-C₅haloalkyl;
R₃ is chosen from H, C₁-C₅alkyl, deuterated C₁-C₅alkyl, C₁-C₅alkoxy, C₁-C₅haloalkyl, C₃-C₇cycloalkyl, and heterocycle, wherein each is independently substituted with 0, 1, 2, or 3 R^{a};
R₄ is C₂₋₆ alkenyl or C₂₋₆ alkynyl, wherein each is independently substituted with 0, 1, 2, or 3 R^{a};
Q is “NH” or “O”;
Ring A is chosen from piperidine, pyrrolidine, bicyclic amine, bridge cyclic amine, and a spirocyclic amine, each of which is optionally substituted with halogen, C₁-C₅alkyl, or C₁-C₅alkoxy; and
R^{a} is independently chosen from hydrogen, deuterium, hydroxyl, halogen, amino group, alkylamino group, dialkylamino group, —CN, C₁₋₆ alkyl, deuterated C₁₋₆ alkyl, C₁₋₆ alkoxyl, deuterated C₁₋₆ alkoxyl, C₁₋₆ haloalkyl, C₁₋₆ haloalkoxyl, C₃₋₆ cycloalkyl, heterocycle, heterocycloalkyl, aryl, and heteroaryl.

2. The compound according to claim 1, wherein X is chosen from

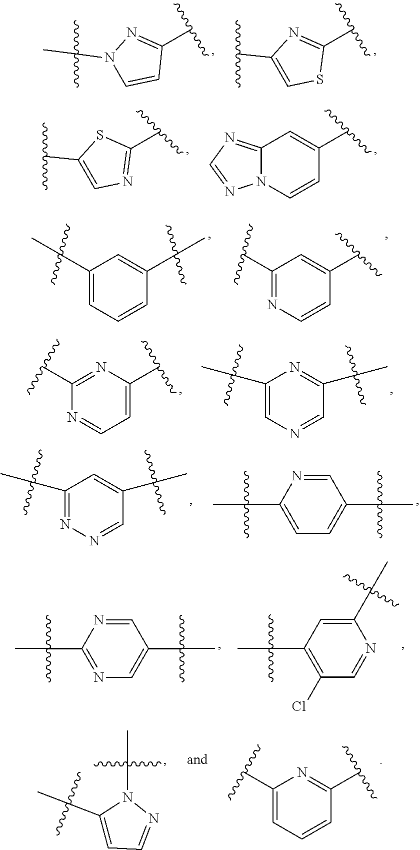

3. (canceled)

4. The compound according to claim 1, wherein Y is absent or is chosen from

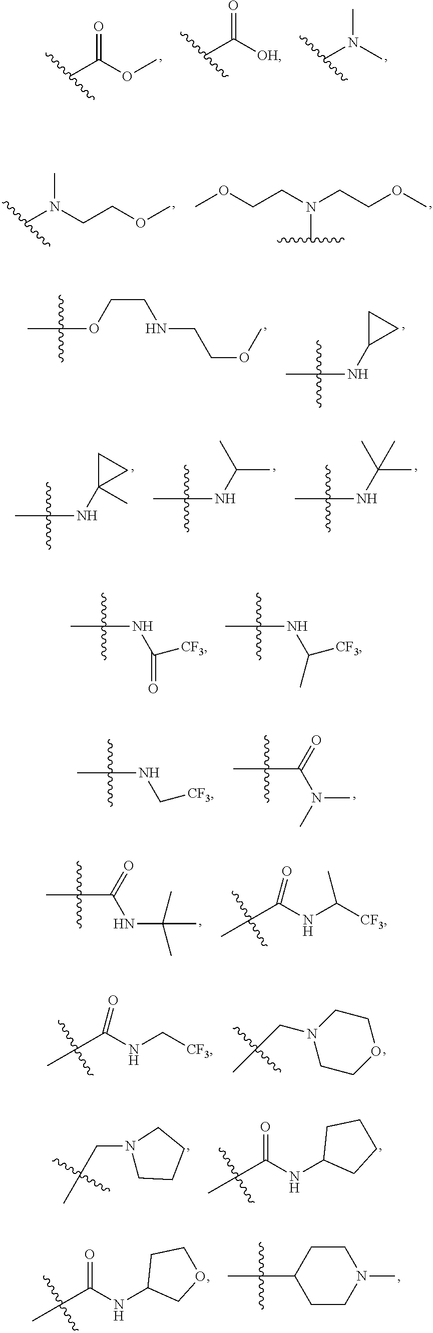

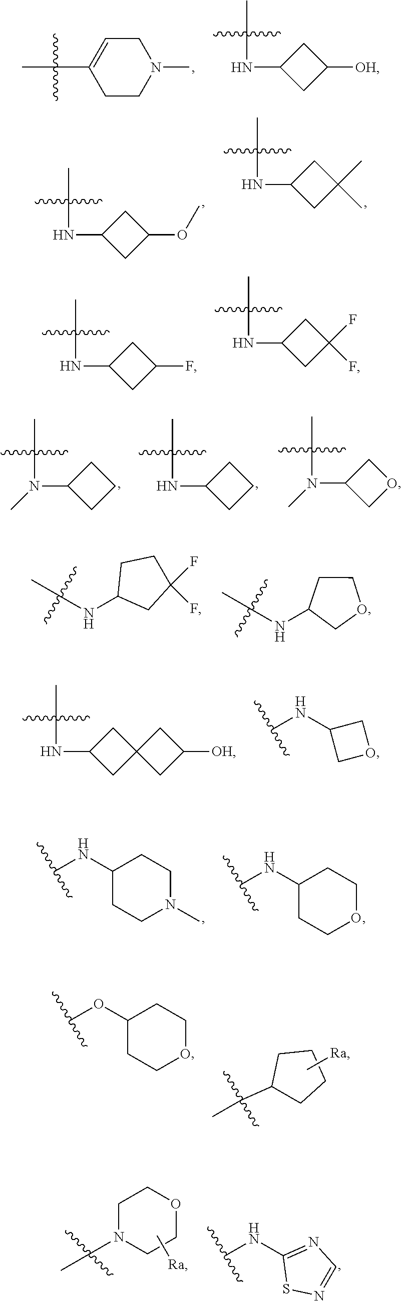

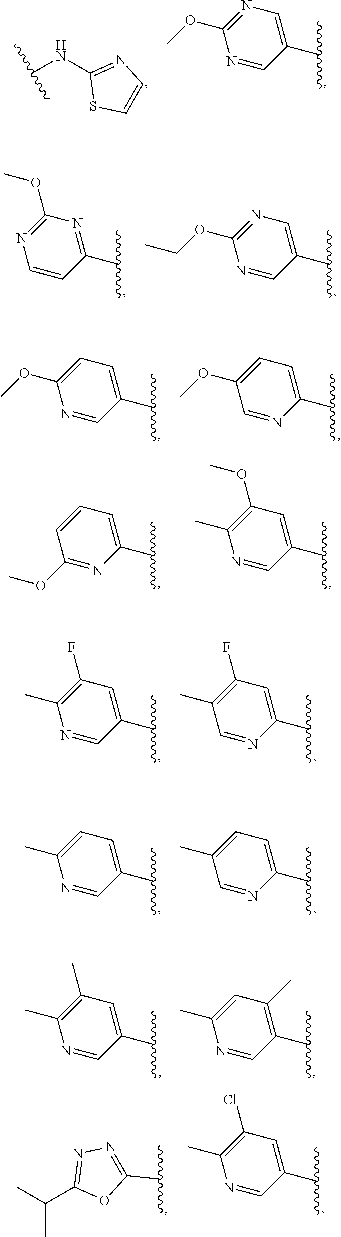

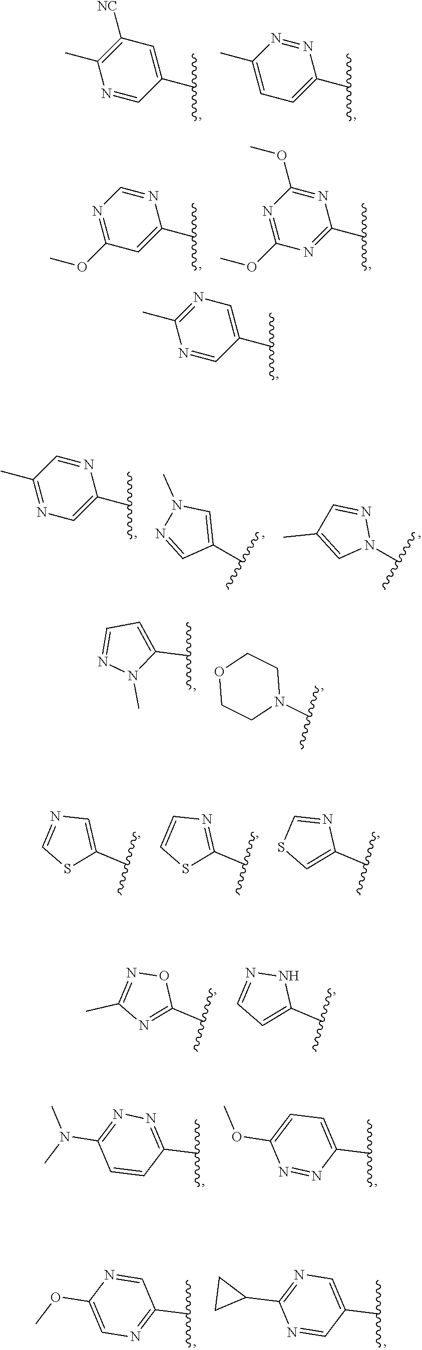

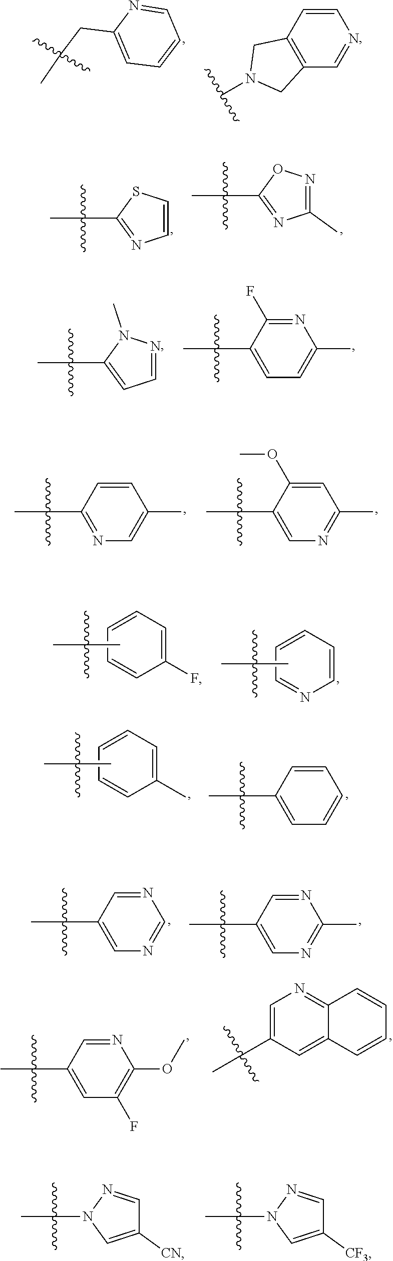

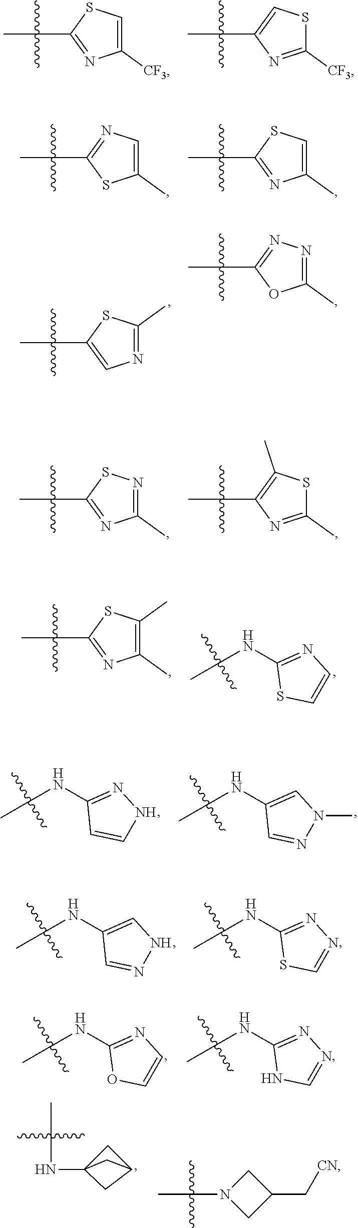

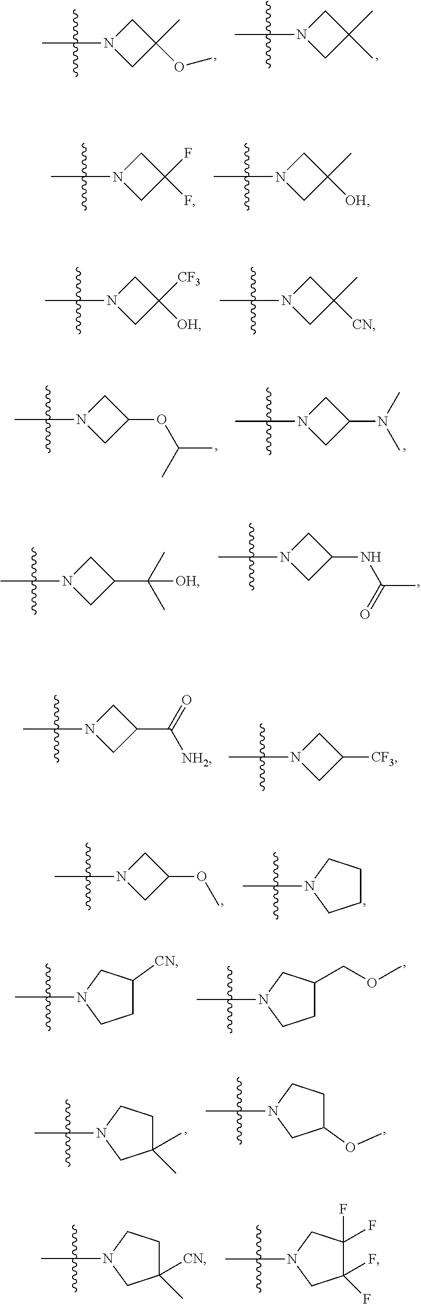

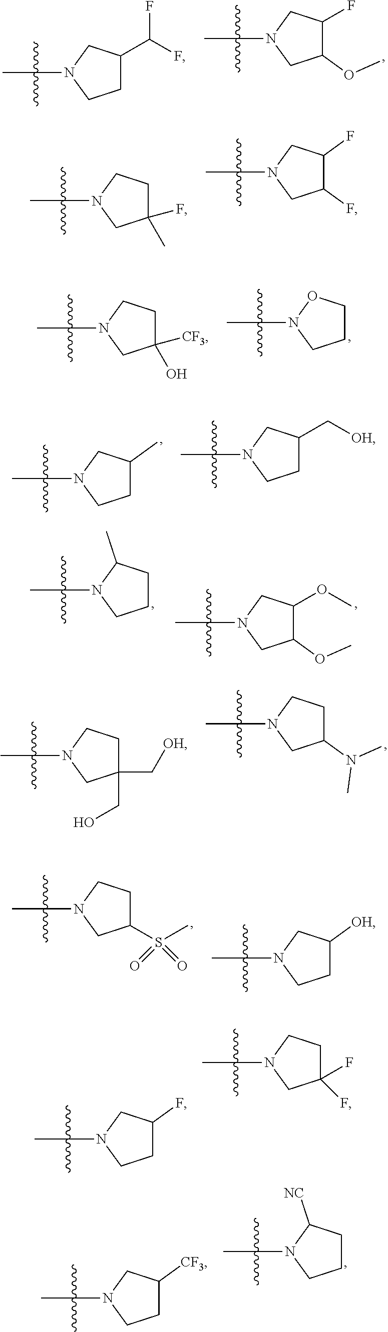

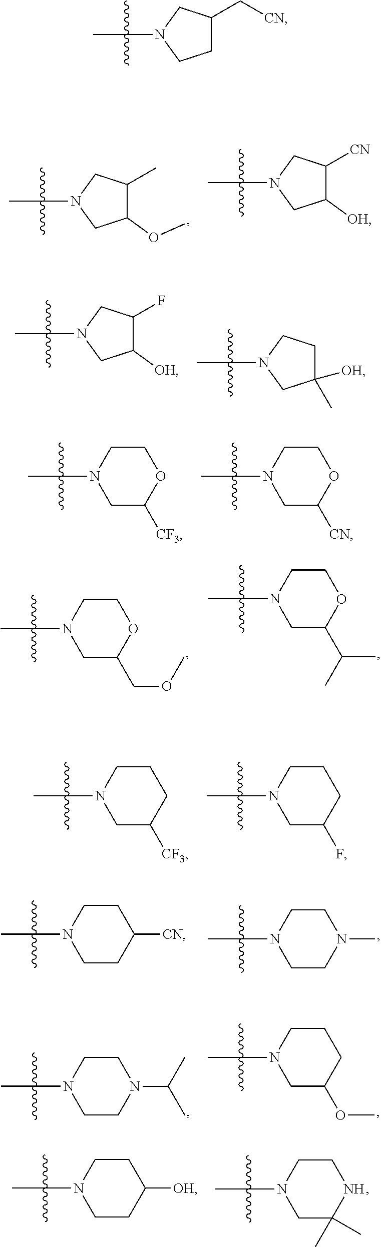

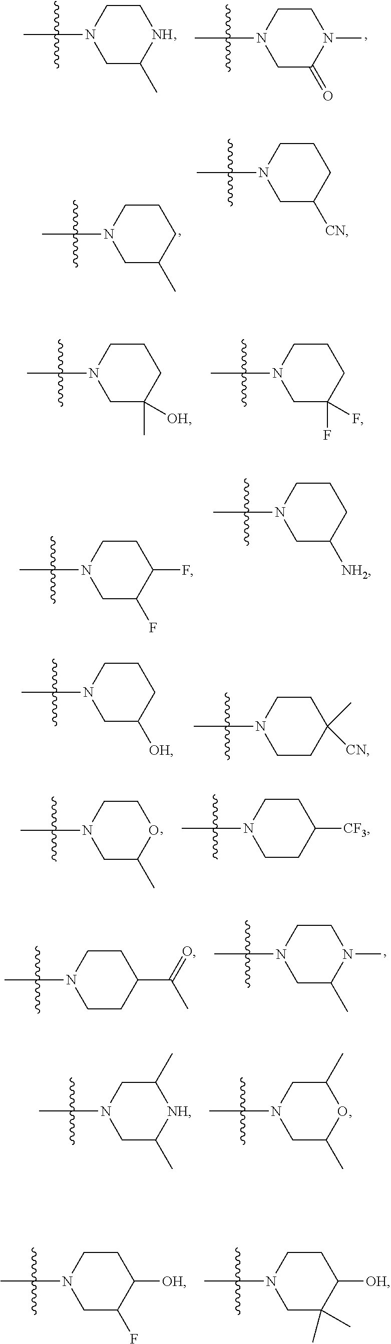

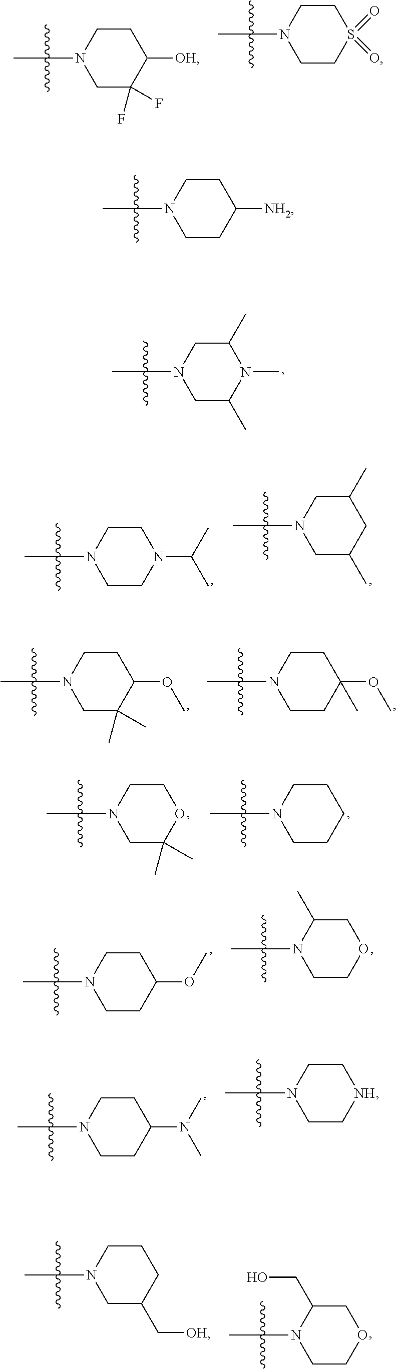

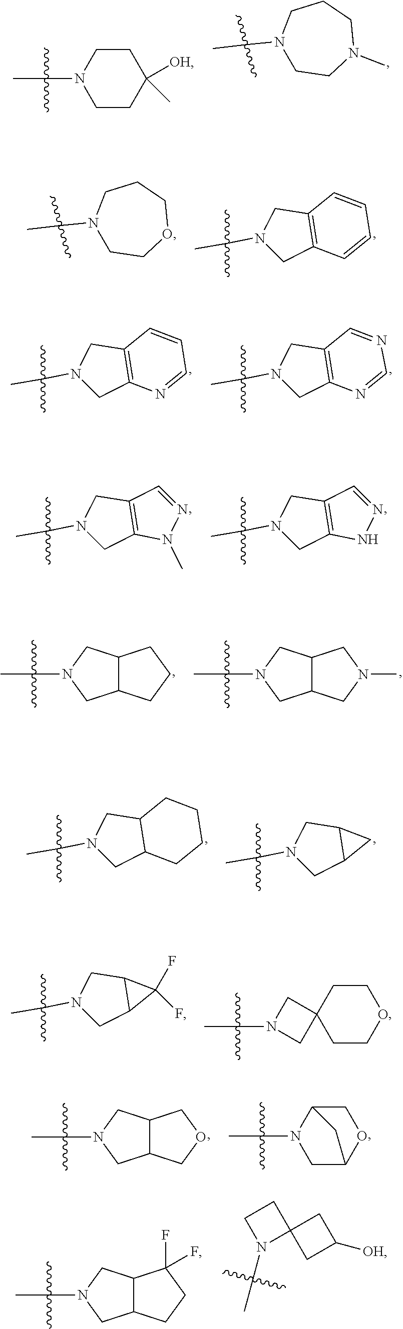

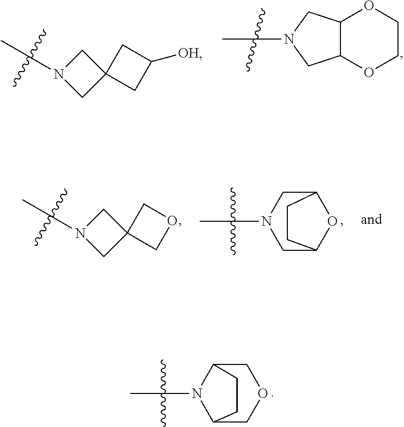 [SMILES: *N1CC2(CC(O)C2)C1 ; *N1CC2OCCOC2C1 ; *N1CC2(COC2)C1 ; *N1CC2CCC(C1)O2 ; *N1C2CCC1COC2]

5. The compound according to claim 1, wherein R₁ and R₂ are each independently chosen from hydrogen, halogen, —CN, C₁₋₄alkyl, and C₁₋₄haloalkyl.

6. (canceled)

7. (canceled)

8. (canceled)

9. (canceled)

10. (canceled)

11. (canceled)

12. (canceled)

13. The compound according to claim 1, wherein R₃ is a C₁₋₄ alkyl.

14. The compound according to claim 13, wherein the C₁₋₄ alkyl is substituted with deuterium, halogen, hydroxyl, C₁₋₄ alkoxyl, amino group, alkylamino group, dialkylamino group, morpholinyl, piperidinyl, pyrrolidinyl, and or azetidinyl.

15. (canceled)

16. (canceled)

17. The compound according to claim 1, wherein R₄ is a C₂₋₆ alkenyl substituted with C₁₋₆ alkyl, C₁₋₄ alkoxyl, halogen, —CN, or a dialkylamino group.

18. The compound according to claim 1, wherein R₄ is a C₂₋₆ alkynyl substituted with C₁₋₆ alkyl, C₁₋₄ alkoxyl, halogen, —CN, or a dialkylamino group.

19. The compound according to claim 1, wherein R₄ is chosen from —CH═CH₂, 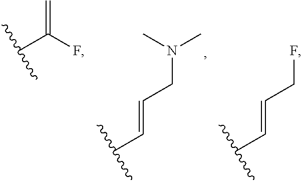
—C≡C—CH₃, —CH═CH—CH₃, and

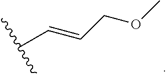 [SMILES: */C=C/COC]

20. (canceled)

21. (canceled)

22. (canceled)

23. (canceled)

24. (canceled)

25. (canceled)

26. (canceled)

27. The compound according to claim 1, wherein ring A is chosen from

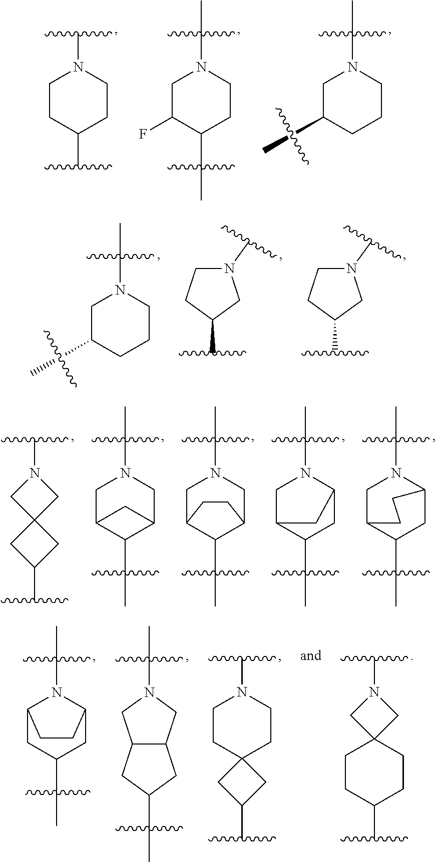

28. (canceled)

29. (canceled)

30. (canceled)

31. The compound according to claim 1, wherein R₃ is chosen from CD₃, —CH₃, —CH₂—CH₃, —CH(CH₃)₂, —CH₂CH₂OCH₃, —CHF₂, —CH₂CHF₂, cyclopropyl, —CH₂CH₂OH, CH₂CH₂N(CH₃)₂, CH₂CH₂NHCH₃,

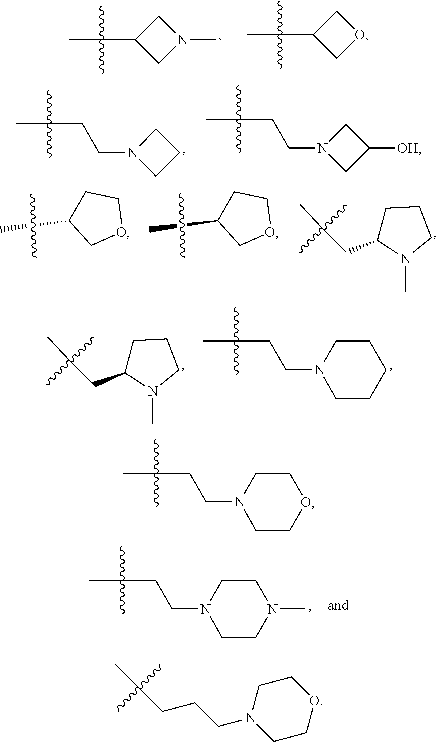

32. The compound according to claim 1, wherein the compound is chosen from:
1-(4-((4-((2-fluoro-4-((1-(2-methoxypyrimidin-5-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((4-(5-isopropyl-1,3,4-oxadiazol-2-yl)thiazol-2-yl)oxy)phenyl)amino)-7-methoxyquinazolin-6-yl)amino)piperidin-1-yl)prop-2-en-1-one;
1-(4-((4-((2-fluoro-4-((5-(6-methylpyridin-3-yl)thiazol-2-yl)oxy)phenyl)amino)-7-methoxyquinazolin-6-yl)amino)piperidin-1-yl)prop-2-en-1-one;
1-(4-((4-((2-fluoro-4-((4-(6-methylpyridin-3-yl)thiazol-2-yl)oxy)phenyl)amino)-7-methoxyquinazolin-6-yl)amino)piperidin-1-yl)prop-2-en-1-one;
1-(4-((4-((4-([1,2,4]triazolo[1,5-a]pyridin-7-yloxy)-3-chlorophenyl)amino)-7-methoxyquinazolin-6-yl)amino)piperidin-1-yl)prop-2-en-1-one;
(S)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one;
(R)-1-(3-((4-((2-fluoro-4-((1-(2-methoxypyrimidin-5-yl)-1H-pyrazol-3-yl)oxy)phenyl)amino)-7-methoxyquinazolin-6-yl)amino)pyrrolidin-1-yl)prop-2-en-1-one;
1-(4-((4-((4-((1-(5-chloro-6-methylpyridin-3-yl)-1H-pyrazol-3-yl)oxy)-2-fluorophenyl)amino)-7-methoxyquinazolin-6-yl)amino)piperidin-1-yl)prop-2-en-1-one;
5-(3-(4-((6-((1-acryloylpiperidin-4-yl)amino)-7-methoxyquinazolin-4-yl)amino)-3-fluorophenoxy)-1H-pyrazol-1-yl)-2-methylnicotinonitrile;
1-(4-((4-((2-fluoro-4-((1-(6-methoxypyridin-3-yl)-1H-pyrazol-3-yl)oxy)phenyl)amino)-7-methoxyquinazolin-6-yl)amino)piperidin-1-yl)prop-2-en-1-one;
1-(4-((7-ethoxy-4-((2-fluoro-4-((1-(5-fluoro-6-methylpyridin-3-yl)-1H-pyrazol-3-yl)oxy)phenyl)amino)quinazolin-6-yl)amino)piperidin-1-yl)prop-2-en-1-one;
1-(4-((4-((2-fluoro-4-((1-(5-methylpyridin-2-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(4-fluoro-5-methylpyridin-2-yl)-1H-pyrazol-3-yl)oxy)phenyl)amino)-7-methoxyquinazolin-6-yl)amino)piperidin-1-yl)prop-2-en-1-one;
1-(4-((4-((3-chloro-4-((1-(5-methylpyridin-2-yl)-1H-pyrazol-3-yl)oxy)phenyl)amino)-7-methoxyquinazolin-6-yl)amino)piperidin-1-yl)prop-2-en-1-one;
1-(4-((4-((3-chloro-4-((1-(4-fluoro-5-methylpyridin-2-yl)-1H-pyrazol-3-yl)oxy)phenyl)amino)-7-methoxyquinazolin-6-yl)amino)piperidin-1-yl)prop-2-en-1-one;
1-(4-((7-ethoxy-4-((2-fluoro-4-((1-(6-methylpyridin-3-yl)-1H-pyrazol-3-yl)oxy)phenyl)amino)quinazolin-6-yl)amino)piperidin-1-yl)prop-2-en-1-one;
1-(4-((4-((5-chloro-2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((3-chloro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
(E)-4-(dimethylamino)-1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)but-2-en-1-one;
1-(4-((4-((2-fluoro-4-((1-(5-fluoro-6-methylpyridin-3-yl)-1H-pyrazol-3-yl)oxy)phenyl)amino)-7-(2-morpholinoethoxy)quinazolin-6-yl)amino)piperidin-1-yl)prop-2-en-1-one;
2-fluoro-1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)but-2-yn-1-one;
2-fluoro-1-(4-((4-((2-fluoro-4-((1-(6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((4-(1-methyl-1H-pyrazol-5-yl)thiazol-2-yl)oxy)phenyl)amino)-7-methoxyquinazolin-6-yl)amino)piperidin-1-yl)prop-2-en-1-one;
1-(4-((4-((2-fluoro-4-((6′-methyl-[2,3′-bipyridin]-4-yl)oxy)phenyl)amino)-7-methoxyquinazolin-6-yl)amino)piperidin-1-yl)prop-2-en-1-one;
1-(4-((4-((4-((4-(2-ethoxypyrimidin-5-yl)thiazol-2-yl)oxy)-2-fluorophenyl)amino)-7-methoxyquinazolin-6-yl)amino)piperidin-1-yl)prop-2-en-1-one;
1-(4-((4-((2-fluoro-4-((1-(6-methylpyridazin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(6-methoxypyrimidin-4-yl)-1H-pyrazol-3-yl)oxy)phenyl)amino)-7-methoxyquinazolin-6-yl)amino)piperidin-1-yl)prop-2-en-1-one;
1-(4-((4-((4-((1-(4,6-dimethoxy-1,3,5-triazin-2-yl)-1H-pyrazol-3-yl)oxy)-2-fluorophenyl)amino)-7-methoxyquinazolin-6-yl)amino)piperidin-1-yl)prop-2-en-1-one;
1-(4-((4-((2-fluoro-4-((4-(2-methylpyrimidin-5-yl)thiazol-2-yl)oxy)phenyl)amino)-7-methoxyquinazolin-6-yl)amino)piperidin-1-yl)prop-2-en-1-one;
1-(4-((4-((2-fluoro-4-((1-(5-methylpyrazin-2-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((2-(1-methyl-1H-pyrazol-4-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-morpholinopyridin-4-yl)oxy)phenyl)amino)-7-methoxyquinazolin-6-yl)amino)piperidin-1-yl)prop-2-en-1-one;
1-(4-((4-((4-((1-(5,6-dimethylpyridin-3-yl)-1H-pyrazol-3-yl)oxy)-2-fluorophenyl)amino)-7-methoxyquinazolin-6-yl)amino)piperidin-1-yl)prop-2-en-1-one;
1-(4-((4-((4-((1-(4,6-dimethylpyridin-3-yl)-1H-pyrazol-3-yl)oxy)-2-fluorophenyl)amino)-7-methoxyquinazolin-6-yl)amino)piperidin-1-yl)prop-2-en-1-one;
1-(4-((4-((2-fluoro-4-((1-(5-methoxy-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((2-(4-methyl-1H-pyrazol-1-yl)pyrimidin-4-yl)oxy)phenyl)amino)-7-methoxyquinazolin-6-yl)amino)piperidin-1-yl)prop-2-en-1-one;
1-(4-((4-((3-chloro-2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((2-(thiazol-4-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(thiazol-5-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(thiazol-2-yl)pyridin-4-yl)oxy)phenyl)amino)-7-methoxyquinazolin-6-yl)amino)piperidin-1-yl)prop-2-en-1-one;
1-(4-((4-((2-fluoro-4-((4-(3-methyl-1,2,4-oxadiazol-5-yl)pyridin-2-yl)oxy)phenyl)amino)-7-methoxyquinazolin-6-yl)amino)piperidin-1-yl)prop-2-en-1-one;
1-(4-((4-((4-((2-(1H-pyrazol-5-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((6-methyl-[3,4′-bipyridin]-2′-yl)oxy)phenyl)amino)-7-methoxyquinazolin-6-yl)amino)piperidin-1-yl)prop-2-en-1-one;
1-(4-((4-((2-fluoro-4-((1-(2-methoxypyrimidin-4-yl)-1H-pyrazol-3-yl)oxy)phenyl)amino)-7-methoxyquinazolin-6-yl)amino)piperidin-1-yl)prop-2-en-1-one;
1-(4-((4-((4-((1-(6-(dimethylamino)pyridazin-3-yl)-1H-pyrazol-3-yl)oxy)-2-fluorophenyl)amino)-7-methoxyquinazolin-6-yl)amino)piperidin-1-yl)prop-2-en-1-one;
1-(4-((4-((2-fluoro-4-((1-(6-methoxypyridin-3-yl)-1H-pyrazol-3-yl)oxy)phenyl)amino)-7-(methoxy-d3)quinazolin-6-yl)amino)piperidin-1-yl)prop-2-en-1-one;
1-(4-((4-((2-fluoro-4-((1-(6-methoxypyridazin-3-yl)-1H-pyrazol-3-yl)oxy)phenyl)amino)-7-methoxyquinazolin-6-yl)amino)piperidin-1-yl)prop-2-en-1-one;
1-(4-((4-((4-((4-(2-cyclopropylpyrimidin-5-yl)thiazol-2-yl)oxy)-2-fluorophenyl)amino)-7-methoxyquinazolin-6-yl)amino)piperidin-1-yl)prop-2-en-1-one;
1-(4-((4-((2-fluoro-4-((6-(1-methyl-1H-pyrazol-4-yl)pyridin-2-yl)oxy)phenyl)amino)-7-methoxyquinazolin-6-yl)amino)piperidin-1-yl)prop-2-en-1-one;
1-(4-((4-((2-fluoro-4-((4-(2-methoxypyrimidin-5-yl)thiazol-2-yl)oxy)phenyl)amino)-7-methoxyquinazolin-6-yl)amino)piperidin-1-yl)prop-2-en-1-one;
1-(4-((4-((3-fluoro-4-((1-(2-methoxypyrimidin-5-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((4-(6-methoxypyridin-3-yl)thiazol-2-yl)oxy)phenyl)amino)-7-methoxyquinazolin-6-yl)amino)piperidin-1-yl)prop-2-en-1-one;
1-(4-((4-((2-fluoro-4-((4-(6-methoxypyridazin-3-yl)thiazol-2-yl)oxy)phenyl)amino)-7-methoxyquinazolin-6-yl)amino)piperidin-1-yl)prop-2-en-1-one;
1-(4-((4-((2-fluoro-4-((1-(6-methoxypyrimidin-4-yl)-1H-pyrazol-3-yl)oxy)phenyl)amino)-7-(methoxy-d3)quinazolin-6-yl)amino)piperidin-1-yl)prop-2-en-1-one;
1-(4-((4-((2-fluoro-4-((1-(6-methoxypyridin-2-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(5-methoxypyridin-2-yl)-1H-pyrazol-3-yl)oxy)phenyl)amino)-7-methoxyquinazolin-6-yl)amino)piperidin-1-yl)prop-2-en-1-one;
(E)-4-fluoro-1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)but-2-en-1-one;
(E)-1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)-4-methoxybut-2-en-1-one;
1-(7-((4-((2-fluoro-4-((1-(5-fluoro-6-methylpyridin-3-yl)-1H-pyrazol-3-yl)oxy)phenyl)amino)-7-methoxyquinazolin-6-yl)amino)-2-azaspiro[3.5]nonan-2-yl)prop-2-en-1-one;
(E)-1-(4-((4-((2-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)but-2-en-1-one;
1-(7-((4-((2-fluoro-4-((1-(6-methylpyridin-3-yl)-1H-pyrazol-3-yl)oxy)phenyl)amino)-7-methoxyquinazolin-6-yl)amino)-2-azaspiro[3.5]nonan-2-yl)prop-2-en-1-one;
1-((3S,4S)-3-fluoro-4-((4-((2-fluoro-4-((1-(6-methylpyridin-3-yl)-1H-pyrazol-3-yl)oxy)phenyl)amino)-7-methoxyquinazolin-6-yl)oxy)piperidin-1-yl)prop-2-en-1-one;
1-((3R,4R)-3-fluoro-4-((4-((2-fluoro-4-((1-(6-methylpyridin-3-yl)-1H-pyrazol-3-yl)oxy)phenyl)amino)-7-methoxyquinazolin-6-yl)oxy)piperidin-1-yl)prop-2-en-1-one;
1-((3R,4S)-3-fluoro-4-((4-((2-fluoro-4-((1-(6-methylpyridin-3-yl)-1H-pyrazol-3-yl)oxy)phenyl)amino)-7-methoxyquinazolin-6-yl)oxy)piperidin-1-yl)prop-2-en-1-one;
1-((3S,4R)-3-fluoro-4-((4-((2-fluoro-4-((1-(6-methylpyridin-3-yl)-1H-pyrazol-3-yl)oxy)phenyl)amino)-7-methoxyquinazolin-6-yl)oxy)piperidin-1-yl)prop-2-en-1-one;
1-((1R,3r,5S)-3-((4-((2-fluoro-4-((1-(6-methylpyridin-3-yl)-1H-pyrazol-3-yl)oxy)phenyl)amino)-7-methoxyquinazolin-6-yl)oxy)-8-azabicyclo[3.2.1]octan-8-yl)prop-2-en-1-one;
1-((1R,3s,5S)-3-((4-((2-fluoro-4-((1-(6-methylpyridin-3-yl)-1H-pyrazol-3-yl)oxy)phenyl)amino)-7-methoxyquinazolin-6-yl)oxy)-8-azabicyclo[3.2.1]octan-8-yl)prop-2-en-1-one;
1-((1R,5S,8s)-8-((4-((2-fluoro-4-((1-(6-methylpyridin-3-yl)-1H-pyrazol-3-yl)oxy)phenyl)amino)-7-methoxyquinazolin-6-yl)oxy)-3-azabicyclo[3.2.1]octan-3-yl)prop-2-en-1-one;
1-(6-((4-((2-fluoro-4-((1-(6-methylpyridin-3-yl)-1H-pyrazol-3-yl)oxy)phenyl)amino)-7-methoxyquinazolin-6-yl)oxy)-3-azabicyclo[3.1.1]heptan-3-yl)prop-2-en-1-one;
(S)-1-(3-((4-((2-fluoro-4-((1-(6-methylpyridin-3-yl)-1H-pyrazol-3-yl)oxy)phenyl)amino)-7-methoxyquinazolin-6-yl)oxy)piperidin-1-yl)prop-2-en-1-one;
(R)-1-(3-((4-((2-fluoro-4-((1-(6-methylpyridin-3-yl)-1H-pyrazol-3-yl)oxy)phenyl)amino)-7-methoxyquinazolin-6-yl)oxy)piperidin-1-yl)prop-2-en-1-one;
(S)-1-(3-((4-((2-fluoro-4-((1-(6-methylpyridin-3-yl)-1H-pyrazol-3-yl)oxy)phenyl)amino)-7-methoxyquinazolin-6-yl)amino)piperidin-1-yl)prop-2-en-1-one (R)-1-(3-((4-((2-fluoro-4-((1-(6-methylpyridin-3-yl)-1H-pyrazol-3-yl)oxy)phenyl)amino)-7-methoxyquinazolin-6-yl)amino)piperidin-1-yl)prop-2-en-1-one;
1-((1R,3r,5S)-3-((4-((2-fluoro-4-((1-(6-methylpyridin-3-yl)-1H-pyrazol-3-yl)oxy)phenyl)amino)-7-methoxyquinazolin-6-yl)amino)-8-azabicyclo[3.2.1]octan-8-yl)prop-2-en-1-one;
1-((1R,3s,5S)-3-((4-((2-fluoro-4-((1-(6-methylpyridin-3-yl)-1H-pyrazol-3-yl)oxy)phenyl)amino)-7-methoxyquinazolin-6-yl)amino)-8-azabicyclo[3.2.1]octan-8-yl)prop-2-en-1-one;
1-(6-((4-((2-fluoro-4-((1-(2-methoxypyrimidin-5-yl)-1H-pyrazol-3-yl)oxy)phenyl)amino)-7-methoxyquinazolin-6-yl)oxy)-2-azaspiro[3.3]heptan-2-yl)prop-2-en-1-one;
(R)-1-(3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidin-1-yl)prop-2-en-1-one;
(S)-1-(3-((4-((2-fluoro-4-((1-(2-methoxypyrimidin-5-yl)-1H-pyrazol-3-yl)oxy)phenyl)amino)-7-methoxyquinazolin-6-yl)oxy)pyrrolidin-1-yl)prop-2-en-1-one;
1-(4-((4-((2-fluoro-4-((2-((1-methyl-1H-pyrazol-4-yl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-((1-methyl-1H-pyrazol-3-yl)amino)pyridin-4-yl)oxy)phenyl)amino)-7-methoxyquinazolin-6-yl)amino)piperidin-1-yl)prop-2-en-1-one;
1-(6-((4-((2-fluoro-4-((1-(2-methoxypyrimidin-5-yl)-1H-pyrazol-3-yl)oxy)phenyl)amino)-7-methoxyquinazolin-6-yl)amino)-2-azaspiro[3.3]heptan-2-yl)prop-2-en-1-one;
1-(4-((4-((2-fluoro-4-((2-(thiazol-2-ylamino)pyridin-4-yl)oxy)phenyl)amino)-7-methoxyquinazolin-6-yl)amino)piperidin-1-yl)prop-2-en-1-one;
1-(4-((4-((3-fluoro-4-((1-(5-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((4-(6-methylpyridin-3-yl)thiazol-2-yl)oxy)phenyl)amino)-7-(methoxy-d3)quinazolin-6-yl)amino)piperidin-1-yl)prop-2-en-1-one;
1-(4-((4-((2-fluoro-4-((5-(5-fluoro-6-methylpyridin-3-yl)thiazol-2-yl)oxy)phenyl)amino)-7-methoxyquinazolin-6-yl)amino)piperidin-1-yl)prop-2-en-1-one;
1-(4-((4-((2-fluoro-4-((1-(2-fluoro-6-methylpyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(5-fluoro-6-methoxypyridin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(quinolin-3-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(2-methylpyrimidin-5-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((1-(6-fluoro-5-methylpyridin-2-yl)-1H-pyrazol-3-yl)oxy)phenyl)amino)-7-methoxyquinazolin-6-yl)amino)piperidin-1-yl)prop-2-en-1-one;
1-(4-((4-((2-fluoro-4-((4-(5-fluoro-6-methylpyridin-3-yl)thiazol-2-yl)oxy)phenyl)amino)-7-methoxyquinazolin-6-yl)amino)piperidin-1-yl)prop-2-en-1-one;
1-(4-((4-((3-chloro-4-(pyridin-2-ylmethoxy)phenyl)amino)-7-(2-morpholinoethoxy)quinazolin-6-yl)amino)piperidin-1-yl)prop-2-en-1-one;
1-(4-((4-((3-chloro-4-(pyridin-2-ylmethoxy)phenyl)amino)-7-(2-(dimethylamino)ethoxy)quinazolin-6-yl)amino)piperidin-1-yl)prop-2-en-1-one;
1-(4-((4-((2-fluoro-4-((2-(2-methylthiazol-4-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-methylthiazol-2-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(5-methylthiazol-2-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(piperidin-1-yl)pyridin-4-yl)oxy)phenyl)amino)-7-methoxyquinazolin-6-yl)amino)piperidin-1-yl)prop-2-en-1-one;
1-(4-((4-((4-((2-((2R,6S)-2,6-dimethylmorpholino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(pyrrolidin-1-yl)pyridin-4-yl)oxy)phenyl)amino)-7-methoxyquinazolin-6-yl)amino)piperidin-1-yl)prop-2-en-1-one;
1-(4-((4-((4-((2-(dimethylamino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(4-methoxypiperidin-1-yl)pyridin-4-yl)oxy)phenyl)amino)-7-methoxyquinazolin-6-yl)amino)piperidin-1-yl)prop-2-en-1-one;
1-(4-((4-((4-((2-((2S,6S)-2,6-dimethylmorpholino)pyridin-4-yl)oxy)-2-fluorophenyl)amino)-7-methoxyquinazolin-6-yl)amino)piperidin-1-yl)prop-2-en-1-one;
1-(4-((4-((4-((2-(1,4-oxazepan-4-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((5-morpholinopyridin-2-yl)methoxy)phenyl)amino)-7-methoxyquinazolin-6-yl)amino)piperidin-1-yl)prop-2-en-1-one;
1-(4-((4-((4-((5-chloro-2-((2R,6S)-2,6-dimethylmorpholino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((1-(5-fluoro-6-methoxypyridin-3-yl)-1H-pyrazol-5-yl)oxy)phenyl)amino)-7-methoxyquinazolin-6-yl)amino)piperidin-1-yl)prop-2-en-1-one;
1-(4-((4-((4-((5-((2R,6S)-2,6-dimethylmorpholino)pyrimidin-2-yl)oxy)-2-fluorophenyl)amino)-7-methoxyquinazolin-6-yl)amino)piperidin-1-yl)prop-2-en-1-one;
1-(4-((4-((4-((5-((2R,6S)-2,6-dimethylmorpholino)pyridin-2-yl)oxy)-2-fluorophenyl)amino)-7-methoxyquinazolin-6-yl)amino)piperidin-1-yl)prop-2-en-1-one;
1-(4-((4-((4-((2-(1,1-dioxidothiomorpholino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(4-methylpiperazin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-methoxypyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-methoxypyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-((2-methoxyethyl)(methyl)amino)pyridin-4-yl)oxy)phenyl)amino)-7-methoxyquinazolin-6-yl)amino)piperidin-1-yl)prop-2-en-1-one;
1-(4-((4-((4-((2-(bis(2-methoxyethyl)amino)pyridin-4-yl)oxy)-2-fluorophenyl)amino)-7-methoxyquinazolin-6-yl)amino)piperidin-1-yl)prop-2-en-1-one;
1-(4-((4-((4-((2-(2-oxa-6-azaspiro[3.3]heptan-6-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-methoxyazetidin-1-yl)pyridin-4-yl)oxy)phenyl)amino)-7-methoxyquinazolin-6-yl)amino)piperidin-1-yl)prop-2-en-1-one;
1-(4-((4-((4-((2-(4-acetylpiperazin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(4-hydroxypiperidin-1-yl)pyridin-4-yl)oxy)phenyl)amino)-7-methoxyquinazolin-6-yl)amino)piperidin-1-yl)prop-2-en-1-one;
1-(4-((4-((4-((2-(4-(dimethylamino)piperidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(piperazin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(2-(hydroxymethyl)morpholino)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(2-(hydroxymethyl)morpholino)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-(hydroxymethyl)morpholino)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-(hydroxymethyl)morpholino)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-hydroxy-4-methylpiperidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-methoxy-4-methylpiperidin-1-yl)pyridin-4-yl)oxy)phenyl)amino)-7-methoxyquinazolin-6-yl)amino)piperidin-1-yl)prop-2-en-1-one;
1-(4-((4-((4-((2-(8-oxa-3-azabicyclo[3.2.1]octan-3-yl)pyridin-4-yl)oxy)-2-fluorophenyl)amino)-7-methoxyquinazolin-6-yl)amino)piperidin-1-yl)prop-2-en-1-one;
1-(4-((4-((4-((2-((1S,4S)-2-oxa-5-azabicyclo[2.2.1]heptan-5-yl)pyridin-4-yl)oxy)-2-fluorophenyl)amino)-7-methoxyquinazolin-6-yl)amino)piperidin-1-yl)prop-2-en-1-one;
(S)-1-(4-((4-((2-fluoro-4-((2-(2-methylmorpholino)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(2-methylmorpholino)pyridin-4-yl)oxy)phenyl)amino)-7-methoxyquinazolin-6-yl)amino)piperidin-1-yl)prop-2-en-1-one;
1-(4-((4-((4-((2-((1R,4R)-2-oxa-5-azabicyclo[2.2.1]heptan-5-yl)pyridin-4-yl)oxy)-2-fluorophenyl)amino)-7-methoxyquinazolin-6-yl)amino)piperidin-1-yl)prop-2-en-1-one;
(S)-1-(4-((4-((2-fluoro-4-((2-(3-methylmorpholino)pyridin-4-yl)oxy)phenyl)amino)-7-methoxyquinazolin-6-yl)amino)piperidin-1-yl)prop-2-en-1-one;
1-(4-((4-((4-((2-(3-oxa-8-azabicyclo[3.2.1]octan-8-yl)pyridin-4-yl)oxy)-2-fluorophenyl)amino)-7-methoxyquinazolin-6-yl)amino)piperidin-1-yl)prop-2-en-1-one;
(R)-1-(4-((4-((2-fluoro-4-((2-(3-methylmorpholino)pyridin-4-yl)oxy)phenyl)amino)-7-methoxyquinazolin-6-yl)amino)piperidin-1-yl)prop-2-en-1-one;
1-(4-((4-((4-((2-(2,2-dimethylmorpholino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-hydroxyazetidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-(hydroxymethyl)azetidin-1-yl)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)azetidine-3-carbonitrile;
1-(4-((4-((2-fluoro-4-((2-(3-(methoxymethyl)azetidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-(methylsulfonyl)azetidin-1-yl)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)azetidine-3-carboxamide;
N-(1-(4-(4-((6-((1-acryloylpiperidin-4-yl)amino)-7-methoxyquinazolin-4-yl)amino)-3-fluorophenoxy)pyridin-2-yl)azetidin-3-yl)acetamide;
1-(4-((4-((2-fluoro-4-((2-(3-(2-hydroxypropan-2-yl)azetidin-1-yl)pyridin-4-yl)oxy)phenyl)amino)-7-methoxyquinazolin-6-yl)amino)piperidin-1-yl)prop-2-en-1-one;
1-(4-((4-((4-((2-(3-(dimethylamino)azetidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-isopropoxyazetidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-hydroxy-3-methylpyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-(trifluoromethyl)pyrrolidin-1-yl)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)pyrrolidine-3-carbonitrile;
1-(4-((4-((4-((2-((2R,6S)-2,6-dimethylmorpholino)pyridin-4-yl)oxy)-2-fluorophenyl)amino)-7-methoxyquinazolin-6-yl)oxy)piperidin-1-yl)prop-2-en-1-one;
1-(4-((4-((4-((2-(3,3-dimethylpyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-((3R,5S)-3,5-dimethylpiperazin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(3,3-bis(hydroxymethyl)pyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((3R,5S)-3,4,5-trimethylpiperazin-1-yl)pyridin-4-yl)oxy)phenyl)amino)-7-methoxyquinazolin-6-yl)oxy)piperidin-1-yl)prop-2-en-1-one;
1-(4-((4-((2-fluoro-4-((2-(4-isopropylpiperazin-1-yl)pyridin-4-yl)oxy)phenyl)amino)-7-methoxyquinazolin-6-yl)oxy)piperidin-1-yl)prop-2-en-1-one;
1-(4-((4-((4-((2-((3S,5R)-3,5-dimethylpiperidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(4-methoxy-3,3-dimethylpiperidin-1-yl)pyridin-4-yl)oxy)phenyl)amino)-7-methoxyquinazolin-6-yl)amino)piperidin-1-yl)prop-2-en-1-one;
1-(4-((4-((4-((2-(6,6-difluoro-3-azabicyclo[3.1.0]hexan-3-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-(trifluoromethyl)azetidin-1-yl)pyridin-4-yl)oxy)phenyl)amino)-7-methoxyquinazolin-6-yl)amino)piperidin-1-yl)prop-2-en-1-one;
(R)-1-(4-((4-((4-((2-(3-(dimethylamino)pyrrolidin-1-yl)pyridin-4-yl)oxy)-2-fluorophenyl)amino)-7-methoxyquinazolin-6-yl)oxy)piperidin-1-yl)prop-2-en-1-one;
1-(4-((4-((4-((2-((3R,4S)-3,4-dimethoxypyrrolidin-1-yl)pyridin-4-yl)oxy)-2-fluorophenyl)amino)-7-methoxyquinazolin-6-yl)oxy)piperidin-1-yl)prop-2-en-1-one;
(R)-1-(4-((4-((2-fluoro-4-((2-(3-methoxypiperidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-((3aR,6aS)-tetrahydro-1H-furo[3,4-c]pyrrol-5(3H)-yl)pyridin-4-yl)oxy)phenyl)amino)-7-methoxyquinazolin-6-yl)oxy)piperidin-1-yl)prop-2-en-1-one;
1-(4-((4-((4-((5-chloro-2-((2R,6S)-2,6-dimethylmorpholino)pyridin-4-yl)oxy)-2-fluorophenyl)amino)-7-methoxyquinazolin-6-yl)oxy)piperidin-1-yl)prop-2-en-1-one;
4-(4-((6-((1-acryloylpiperidin-4-yl)amino)-7-methoxyquinazolin-4-yl)amino)-3-fluorophenoxy)-N-(tert-butyl)picolinamide;
N-(7-ethoxy-4-((2-fluoro-4-((2-(4-methylthiazol-2-yl)pyridin-4-yl)oxy)phenyl)amino)quinazolin-6-yl)acrylamide;
N-(4-((4-((2-((2S,6R)-2,6-dimethylmorpholino)pyridin-4-yl)oxy)-2-fluorophenyl)amino)-7-ethoxyquinazolin-6-yl)acrylamide;
1-(4-((4-((2-fluoro-4-((2-((3R,4S)-3-fluoro-4-hydroxypiperidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-hydroxy-3,3-dimethylpiperidin-1-yl)pyridin-4-yl)oxy)phenyl)amino)-7-methoxyquinazolin-6-yl)amino)piperidin-1-yl)prop-2-en-1-one;
1-(4-((4-((4-((2-(3,3-difluoro-4-hydroxypiperidin-1-yl)pyridin-4-yl)oxy)-2-fluorophenyl)amino)-7-methoxyquinazolin-6-yl)amino)piperidin-1-yl)prop-2-en-1-one;
(R)-1-(4-((4-((2-fluoro-4-((2-(3-hydroxypyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-hydroxypyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(6-hydroxy-2-azaspiro[3.3]heptan-2-yl)pyridin-4-yl)oxy)phenyl)amino)-7-methoxyquinazolin-6-yl)amino)piperidin-1-yl)prop-2-en-1-one;
1-(4-((4-((4-((2-(4,5-dimethylthiazol-2-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(2,5-dimethylthiazol-4-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(4-aminopiperidin-1-yl)pyridin-4-yl)oxy)-2-fluorophenyl)amino)-7-methoxyquinazolin-6-yl)amino)piperidin-1-yl)prop-2-en-1-one;
(S)-1-(4-((4-((4-((2-(3,4-dimethylpiperazin-1-yl)pyridin-4-yl)oxy)-2-fluorophenyl)amino)-7-methoxyquinazolin-6-yl)amino)piperidin-1-yl)prop-2-en-1-one;
(R)-1-(4-((4-((4-((2-(3,4-dimethylpiperazin-1-yl)pyridin-4-yl)oxy)-2-fluorophenyl)amino)-7-methoxyquinazolin-6-yl)amino)piperidin-1-yl)prop-2-en-1-one;
4-(4-((6-((1-acryloylpiperidin-4-yl)amino)-7-methoxyquinazolin-4-yl)amino)-3-fluorophenoxy)-N-methylpicolinamide;
4-(4-((6-((1-acryloylpiperidin-4-yl)amino)-7-methoxyquinazolin-4-yl)amino)-3-fluorophenoxy)-N,N-dimethylpicolinamide;
1-(4-((4-((2-fluoro-4-((2-(oxetan-3-ylamino)pyridin-4-yl)oxy)phenyl)amino)-7-methoxyquinazolin-6-yl)amino)piperidin-1-yl)prop-2-en-1-one;
1-(4-((4-((2-fluoro-4-((2-((1-methylpiperidin-4-yl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-((tetrahydro-2H-pyran-4-yl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-(1-methylpiperidin-4-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-isopropylpiperazin-1-yl)pyridin-4-yl)oxy)phenyl)amino)-7-methoxyquinazolin-6-yl)amino)piperidin-1-yl)prop-2-en-1-one;
1-(4-((4-((4-((2-(6,6-difluoro-3-azabicyclo[3.1.0]hexan-3-yl)pyridin-4-yl)oxy)-2-fluorophenyl)amino)-7-methoxyquinazolin-6-yl)oxy)piperidin-1-yl)prop-2-en-1-one;
1-(4-((4-((4-((2-((3R,4S)-3,4-dimethoxypyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((3aR,6aS)-tetrahydro-1H-furo[3,4-c]pyrrol-5(3H)-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-methyl-1,2,4-oxadiazol-5-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-methyl-1,2,4-thiadiazol-5-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(5-methyl-1,3,4-oxadiazol-2-yl)pyridin-4-yl)oxy)phenyl)amino)-7-methoxyquinazolin-6-yl)amino)piperidin-1-yl)prop-2-en-1-one;
1-(4-((4-((4-((2-(3,3-difluoropyrrolidin-1-yl)pyridin-4-yl)oxy)-2-fluorophenyl)amino)-7-methoxyquinazolin-6-yl)amino)piperidin-1-yl)prop-2-en-1-one;
(R)-1-(4-((4-((2-fluoro-4-((2-(3-fluoropyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-fluoropyrrolidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-methylpyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-methylpyrrolidin-1-yl)pyridin-4-yl)oxy)phenyl)amino)-7-methoxyquinazolin-6-yl)amino)piperidin-1-yl)prop-2-en-1-one;
1-(4-((4-((4-((2-(3-azabicyclo[3.1.0]hexan-3-yl)pyridin-4-yl)oxy)-2-fluorophenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)-4-methylpiperidine-4-carbonitrile;
1-(4-((4-((4-((2-(3,3-difluoroazetidin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(3,3-dimethylazetidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-hydroxy-3-methylazetidin-1-yl)pyridin-4-yl)oxy)phenyl)amino)-7-methoxyquinazolin-6-yl)amino)piperidin-1-yl)prop-2-en-1-one;
1-(4-((4-((4-((2-(7-oxa-2-azaspiro[3.5]nonan-2-yl)pyridin-4-yl)oxy)-2-fluorophenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)-3-methylazetidine-3-carbonitrile;
1-(4-((4-((2-fluoro-4-((2-(3-hydroxy-3-(trifluoromethyl)azetidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-methoxy-3-methylazetidin-1-yl)pyridin-4-yl)oxy)phenyl)amino)-7-methoxyquinazolin-6-yl)amino)piperidin-1-yl)prop-2-en-1-one;
2-(1-(4-(4-((6-((1-acryloylpiperidin-4-yl)amino)-7-methoxyquinazolin-4-yl)amino)-3-fluorophenoxy)pyridin-2-yl)azetidin-3-yl)acetonitrile;
1-(4-((4-((2-fluoro-4-((2-(pyrrolidin-1-ylmethyl)pyridin-4-yl)oxy)phenyl)amino)-7-methoxyquinazolin-6-yl)amino)piperidin-1-yl)prop-2-en-1-one;
1-(4-((4-((2-fluoro-4-((2-(morpholinomethyl)pyridin-4-yl)oxy)phenyl)amino)-7-methoxyquinazolin-6-yl)amino)piperidin-1-yl)prop-2-en-1-one;
methyl 4-(4-((6-((1-acryloylpiperidin-4-yl)amino)-7-methoxyquinazolin-4-yl)amino)-3-fluorophenoxy)picolinate;
4-(4-((6-((1-acryloylpiperidin-4-yl)amino)-7-methoxyquinazolin-4-yl)amino)-3-fluorophenoxy)picolinic acid;
1-(4-((4-((2-fluoro-4-((1′-methyl-1′,2′,3′,6′-tetrahydro-[2,4′-bipyridin]-4-yl)oxy)phenyl)amino)-7-methoxyquinazolin-6-yl)amino)piperidin-1-yl)prop-2-en-1-one;
(R)-1-(4-((4-((2-fluoro-4-((2-(3-hydroxypiperidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-methoxypiperidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-methylpiperidin-1-yl)pyridin-4-yl)oxy)phenyl)amino)-7-methoxyquinazolin-6-yl)amino)piperidin-1-yl)prop-2-en-1-one;
(R)-1-(4-((4-((4-((2-(3-aminopiperidin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(4,4-difluoropiperidin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(3,3-difluoropiperidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-hydroxy-3-methylpiperidin-1-yl)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)piperidine-3-carbonitrile;
(S)-1-(4-((4-((2-fluoro-4-((2-(3-methylpiperidin-1-yl)pyridin-4-yl)oxy)phenyl)amino)-7-methoxyquinazolin-6-yl)amino)piperidin-1-yl)prop-2-en-1-one;
4-(4-(4-((6-((1-acryloylpiperidin-4-yl)amino)-7-methoxyquinazolin-4-yl)amino)-3-fluorophenoxy)pyridin-2-yl)-1-methylpiperazin-2-one;
(R)-1-(4-((4-((2-fluoro-4-((2-(3-methylpiperazin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-methylpiperazin-1-yl)pyridin-4-yl)oxy)phenyl)amino)-7-methoxyquinazolin-6-yl)amino)piperidin-1-yl)prop-2-en-1-one;
1-(4-((4-((4-((2-(3,3-dimethylpiperazin-1-yl)pyridin-4-yl)oxy)-2-fluorophenyl)amino)-7-methoxyquinazolin-6-yl)amino)piperidin-1-yl)prop-2-en-1-one;
(R)-1-(4-((4-((3-chloro-4-((2-(3-methoxypiperidin-1-yl)pyridin-4-yl)oxy)phenyl)amino)-7-methoxyquinazolin-6-yl)amino)piperidin-1-yl)prop-2-en-1-one;
1-(4-((4-((3-chloro-4-((2-(4-methylpiperazin-1-yl)pyridin-4-yl)oxy)phenyl)amino)-7-methoxyquinazolin-6-yl)amino)piperidin-1-yl)prop-2-en-1-one;
1-(4-((4-((3-chloro-4-((2-(4-hydroxypiperidin-1-yl)pyridin-4-yl)oxy)phenyl)amino)-7-methoxyquinazolin-6-yl)amino)piperidin-1-yl)prop-2-en-1-one;
(R)-1-(4-((7-methoxy-4-((4-((2-(3-methoxypiperidin-1-yl)pyridin-4-yl)oxy)phenyl)amino)quinazolin-6-yl)amino)piperidin-1-yl)prop-2-en-1-one;
1-(4-((7-methoxy-4-((4-((2-(4-methylpiperazin-1-yl)pyridin-4-yl)oxy)phenyl)amino)quinazolin-6-yl)amino)piperidin-1-yl)prop-2-en-1-one;
1-(4-((4-((2-fluoro-4-((2-(4-methyl-1H-pyrazol-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-phenylpyridin-4-yl)oxy)phenyl)amino)-7-methoxyquinazolin-6-yl)amino)piperidin-1-yl)prop-2-en-1-one;
1-(4-((4-((2-fluoro-4-((2-(m-tolyl)pyridin-4-yl)oxy)phenyl)amino)-7-methoxyquinazolin-6-yl)amino)piperidin-1-yl)prop-2-en-1-one;
1-(4-((4-((2-fluoro-4-((2-(p-tolyl)pyridin-4-yl)oxy)phenyl)amino)-7-methoxyquinazolin-6-yl)amino)piperidin-1-yl)prop-2-en-1-one;
1-(4-((4-((4-([2,2′-bipyridin]-4-yloxy)-2-fluorophenyl)amino)-7-methoxyquinazolin-6-yl)amino)piperidin-1-yl)prop-2-en-1-one;
1-(4-((4-((4-([2,3′-bipyridin]-4-yloxy)-2-fluorophenyl)amino)-7-methoxyquinazolin-6-yl)amino)piperidin-1-yl)prop-2-en-1-one;
1-(4-((4-((4-([2,4′-bipyridin]-4-yloxy)-2-fluorophenyl)amino)-7-methoxyquinazolin-6-yl)amino)piperidin-1-yl)prop-2-en-1-one;
1-(4-((4-((2-fluoro-4-((2-(hexahydrocyclopenta[c]pyrrol-2(1H)-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(5-methylhexahydropyrrolo[3,4-c]pyrrol-2(1H)-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-((3aR,7aS)-octahydro-2H-isoindol-2-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-methyl-1,4-diazepan-1-yl)pyridin-4-yl)oxy)phenyl)amino)-7-methoxyquinazolin-6-yl)amino)piperidin-1-yl)prop-2-en-1-one;
1-(4-((4-((4-((2-(cyclobutylamino)pyridin-4-yl)oxy)-2-fluorophenyl)amino)-7-methoxyquinazolin-6-yl)amino)piperidin-1-yl)prop-2-en-1-one;
1-(4-((4-((4-((2-(cyclopropylamino)pyridin-4-yl)oxy)-2-fluorophenyl)amino)-7-methoxyquinazolin-6-yl)amino)piperidin-1-yl)prop-2-en-1-one;
1-(4-((4-((4-((2-(tert-butylamino)pyridin-4-yl)oxy)-2-fluorophenyl)amino)-7-methoxyquinazolin-6-yl)amino)piperidin-1-yl)prop-2-en-1-one;
1-(4-((4-((4-((2-(cyclopentylamino)pyridin-4-yl)oxy)-2-fluorophenyl)amino)-7-methoxyquinazolin-6-yl)amino)piperidin-1-yl)prop-2-en-1-one;
(S)-1-(4-((4-((2-fluoro-4-((2-((tetrahydrofuran-3-yl)amino)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-((tetrahydrofuran-3-yl)amino)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(2-methylpyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-(hydroxymethyl)pyrrolidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-(hydroxymethyl)pyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(2-methylpyrrolidin-1-yl)pyridin-4-yl)oxy)phenyl)amino)-7-methoxyquinazolin-6-yl)amino)piperidin-1-yl)prop-2-en-1-one;
1-(4-((4-((4-((2-((3S,4R)-3,4-difluoropyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((4aR,7aS)-hexahydro-6H-[1,4]dioxino[2,3-c]pyrrol-6-yl)pyridin-4-yl)oxy)phenyl)amino)-7-methoxyquinazolin-6-yl)amino)piperidin-1-yl)prop-2-en-1-one;
1-(4-((4-((4-((2-((3R,4R)-3,4-dimethoxypyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-((3S,4S)-3,4-dimethoxypyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((4aR,7aS)-hexahydro-6H-[1,4]dioxino[2,3-c]pyrrol-6-yl)pyrimidin-4-yl)oxy)phenyl)amino)-7-methoxyquinazolin-6-yl)amino)piperidin-1-yl)prop-2-en-1-one;
(R)-1-(4-(4-((6-((1-acryloylpiperidin-4-yl)amino)-7-methoxyquinazolin-4-yl)amino)-3-fluorophenoxy)pyridin-2-yl)pyrrolidine-3-carbonitrile;
(S)-1-(4-(4-((6-((1-acryloylpiperidin-4-yl)amino)-7-methoxyquinazolin-4-yl)amino)-3-fluorophenoxy)pyridin-2-yl)pyrrolidine-3-carbonitrile;
1-(4-((4-((4-((2-((3R,4S)-3,4-dimethoxypyrrolidin-1-yl)pyrimidin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3,3,4,4-tetrafluoropyrrolidin-1-yl)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)-3-methylpyrrolidine-3-carbonitrile;
1-(4-((4-((2-fluoro-4-((2-((3R,4S)-3-fluoro-4-methoxypyrrolidin-1-yl)pyridin-4-yl)oxy)phenyl)amino)-7-methoxyquinazolin-6-yl)amino)piperidin-1-yl)prop-2-en-1-one;
1-(4-((4-((4-((2-((3S,4S)-3,4-difluoropyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(3-fluoro-3-methylpyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(isoxazolidin-2-yl)pyridin-4-yl)oxy)phenyl)amino)-7-methoxyquinazolin-6-yl)amino)piperidin-1-yl)prop-2-en-1-one;
(R)-4-(4-((6-((1-acryloylpiperidin-4-yl)amino)-7-methoxyquinazolin-4-yl)amino)-3-fluorophenoxy)-N-(tetrahydrofuran-3-yl)picolinamide;
4-(4-((6-((1-acryloylpiperidin-4-yl)amino)-7-methoxyquinazolin-4-yl)amino)-3-fluorophenoxy)-N-cyclopentylpicolinamide;
(S)-4-(4-((6-((1-acryloylpiperidin-4-yl)amino)-7-methoxyquinazolin-4-yl)amino)-3-fluorophenoxy)-N-(tetrahydrofuran-3-yl)picolinamide;
(R)-1-(4-((4-((2-fluoro-4-((2-(3-(trifluoromethyl)pyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-(trifluoromethyl)pyrrolidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-hydroxy-3-(trifluoromethyl)pyrrolidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-hydroxy-3-(trifluoromethyl)pyrrolidin-1-yl)pyridin-4-yl)oxy)phenyl)amino)-7-methoxyquinazolin-6-yl)amino)piperidin-1-yl)prop-2-en-1-one;
(R)-1-(4-((4-((4-((2-(3-(difluoromethyl)pyrrolidin-1-yl)pyridin-4-yl)oxy)-2-fluorophenyl)amino)-7-methoxyquinazolin-6-yl)amino)piperidin-1-yl)prop-2-en-1-one;
(S)-1-(4-((4-((4-((2-(3-(difluoromethyl)pyrrolidin-1-yl)pyridin-4-yl)oxy)-2-fluorophenyl)amino)-7-methoxyquinazolin-6-yl)amino)piperidin-1-yl)prop-2-en-1-one;
1-(4-((4-((4-((2-((3aR,6aS)-4,4-difluorohexahydrocyclopenta[c]pyrrol-2(1H)-yl)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((3R,4R)-3-fluoro-4-methoxypyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(2-hydroxy-6-azaspiro[3.4]octan-6-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(2-(trifluoromethyl)thiazol-4-yl)pyridin-4-yl)oxy)phenyl)amino)-7-methoxyquinazolin-6-yl)amino)piperidin-1-yl)prop-2-en-1-one;
1-(4-((4-((4-((2-(cyclobutylamino)pyridin-4-yl)oxy)-2-fluorophenyl)amino)-7-(methoxy-d3)quinazolin-6-yl)amino)piperidin-1-yl)prop-2-en-1-one;
1-(4-((4-((2-fluoro-4-((2-(4-(trifluoromethyl)thiazol-2-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(1-methyl-1H-pyrazol-5-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-(trifluoromethyl)-1H-pyrazol-1-yl)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)-1H-pyrazole-4-carbonitrile;
1-(4-((4-((2-fluoro-4-((2-(pyrimidin-5-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-(2-fluorophenyl)pyridin-4-yl)oxy)phenyl)amino)-7-methoxyquinazolin-6-yl)amino)piperidin-1-yl)prop-2-en-1-one;
1-(4-((4-((2-fluoro-4-((2-(3-fluorophenyl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-fluorophenyl)pyridin-4-yl)oxy)phenyl)amino)-7-methoxyquinazolin-6-yl)amino)piperidin-1-yl)prop-2-en-1-one;
1-(4-((4-((2-fluoro-4-((2-(4-(trifluoromethyl)piperidin-1-yl)pyridin-4-yl)oxy)phenyl)amino)-7-methoxyquinazolin-6-yl)amino)piperidin-1-yl)prop-2-en-1-one;
2-(4-(4-((6-((1-acryloylpiperidin-4-yl)amino)-7-methoxyquinazolin-4-yl)amino)-3-fluorophenoxy)pyridin-2-yl)thiazole-4-carbonitrile;
(R)-1-(4-((4-((2-fluoro-4-((2-(3-(trifluoromethyl)pyrrolidin-1-yl)pyrimidin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-(trifluoromethyl)pyrrolidin-1-yl)pyrimidin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-fluoropyrrolidin-1-yl)pyrimidin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-fluoropyrrolidin-1-yl)pyrimidin-4-yl)oxy)phenyl)amino)-7-methoxyquinazolin-6-yl)amino)piperidin-1-yl)prop-2-en-1-one;
1-(4-((4-((4-((2-((1R,4R)-2-oxa-5-azabicyclo[2.2.1]heptan-5-yl)pyrimidin-4-yl)oxy)-2-fluorophenyl)amino)-7-methoxyquinazolin-6-yl)amino)piperidin-1-yl)prop-2-en-1-one;
(R)-1-(4-((4-((2-fluoro-4-((2-(3-methylpyrrolidin-1-yl)pyrimidin-4-yl)oxy)phenyl)amino)-7-methoxyquinazolin-6-yl)amino)piperidin-1-yl)prop-2-en-1-one;
1-(4-((4-((2-fluoro-4-((2-((3aR,6aS)-tetrahydro-1H-furo[3,4-c]pyrrol-5(3H)-yl)pyrimidin-4-yl)oxy)phenyl)amino)-7-methoxyquinazolin-6-yl)amino)piperidin-1-yl)prop-2-en-1-one;
1-(4-((4-((2-fluoro-4-((2-((tetrahydro-2H-pyran-4-yl)oxy)pyridin-4-yl)oxy)phenyl)amino)-7-methoxyquinazolin-6-yl)amino)piperidin-1-yl)prop-2-en-1-one;
(S)-1-(4-(4-((6-((1-acryloylpiperidin-4-yl)amino)-7-methoxyquinazolin-4-yl)amino)-3-fluorophenoxy)pyridin-2-yl)pyrrolidine-2-carbonitrile;
(R)-1-(4-(4-((6-((1-acryloylpiperidin-4-yl)amino)-7-methoxyquinazolin-4-yl)amino)-3-fluorophenoxy)pyridin-2-yl)pyrrolidine-2-carbonitrile;
1-(4-((4-((4-((2-(cyclobutylamino)pyridin-4-yl)oxy)-2-fluorophenyl)amino)-7-ethoxyquinazolin-6-yl)amino)piperidin-1-yl)prop-2-en-1-one;
1-(4-((4-((2-fluoro-4-((2-((3S,4S)-3-methoxy-4-methylpyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-((3S,4R)-3-fluoro-4-methoxypyrrolidin-1-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-((3R,4R)-3-fluoro-4-methoxypyrrolidin-1-yl)pyridin-4-yl)oxy)phenyl)amino)-7-(methoxy-d3)quinazolin-6-yl)amino)piperidin-1-yl)prop-2-en-1-one;
1-(4-((4-((4-((2-((1,2,4-thiadiazol-5-yl)amino)pyridin-4-yl)oxy)-2-fluorophenyl)amino)-7-methoxyquinazolin-6-yl)amino)piperidin-1-yl)prop-2-en-1-one;
(S)-1-(4-((4-((2-fluoro-4-((2-(3-fluoropiperidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-fluoropiperidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(3-(trifluoromethyl)piperidin-1-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(3-(trifluoromethyl)piperidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(2-isopropylmorpholino)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(2-isopropylmorpholino)pyridin-4-yl)oxy)phenyl)amino)-7-methoxyquinazolin-6-yl)amino)piperidin-1-yl)prop-2-en-1-one;
(R)-1-(4-((4-((2-fluoro-4-((2-(2-(methoxymethyl)morpholino)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(2-(methoxymethyl)morpholino)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-(2-(trifluoromethyl)morpholino)pyridin-4-yl)oxy)phenyl)amino)-7-methoxyquinazolin-6-yl)amino)piperidin-1-yl)prop-2-en-1-one;
(S)-1-(4-((4-((2-fluoro-4-(3-(3-(trifluoromethyl)pyrrolidin-1-yl)phenoxy)phenyl)amino)-7-methoxyquinazolin-6-yl)amino)piperidin-1-yl)prop-2-en-1-one;
(R)-1-(4-((4-((2-fluoro-4-((2-(2-(trifluoromethyl)morpholino)pyridin-4-yl)oxy)phenyl)amino)-7-methoxyquinazolin-6-yl)amino)piperidin-1-yl)prop-2-en-1-one;
(S)-1-(4-(4-((6-((1-acryloylpiperidin-4-yl)amino)-7-methoxyquinazolin-4-yl)amino)-3-fluorophenoxy)pyridin-2-yl)-3-methylpyrrolidine-3-carbonitrile;
(R)-1-(4-(4-((6-((1-acryloylpiperidin-4-yl)amino)-7-methoxyquinazolin-4-yl)amino)-3-fluorophenoxy)pyridin-2-yl)-3-methylpyrrolidine-3-carbonitrile;
1-(4-((4-((4-((2-(5,7-dihydro-6H-pyrrolo[3,4-d]pyrimidin-6-yl)pyridin-4-yl)oxy)-2-fluorophenyl)amino)-7-methoxyquinazolin-6-yl)amino)piperidin-1-yl)prop-2-en-1-one;
(R)-1-(4-((4-((2-fluoro-4-(3-(3-(trifluoromethyl)pyrrolidin-1-yl)phenoxy)phenyl)amino)-7-methoxyquinazolin-6-yl)amino)piperidin-1-yl)prop-2-en-1-one;
1-(4-((4-((4-((2-(1,3-dihydro-2H-pyrrolo[3,4-c]pyridin-2-yl)pyridin-4-yl)oxy)-2-fluorophenyl)amino)-7-methoxyquinazolin-6-yl)amino)piperidin-1-yl)prop-2-en-1-one;
1-(4-((4-((4-((2-(5,7-dihydro-6H-pyrrolo[3,4-b]pyridin-6-yl)pyridin-4-yl)oxy)-2-fluorophenyl)amino)-7-methoxyquinazolin-6-yl)amino)piperidin-1-yl)prop-2-en-1-one;
(R)-4-(4-(4-((6-((1-acryloylpiperidin-4-yl)amino)-7-methoxyquinazolin-4-yl)amino)-3-fluorophenoxy)pyridin-2-yl)morpholine-2-carbonitrile;
(S)-4-(4-(4-((6-((1-acryloylpiperidin-4-yl)amino)-7-methoxyquinazolin-4-yl)amino)-3-fluorophenoxy)pyridin-2-yl)morpholine-2-carbonitrile;
1-(4-((4-((2-fluoro-4-((2-(isoindolin-2-yl)pyridin-4-yl)oxy)phenyl)amino)-7-methoxyquinazolin-6-yl)amino)piperidin-1-yl)prop-2-en-1-one;
(S)-1-(4-((4-((2-fluoro-4-(3-(3-fluoropyrrolidin-1-yl)phenoxy)phenyl)amino)-7-methoxyquinazolin-6-yl)amino)piperidin-1-yl)prop-2-en-1-one;
(S)-2-(1-(4-(4-((6-((1-acryloylpiperidin-4-yl)amino)-7-methoxyquinazolin-4-yl)amino)-3-fluorophenoxy)pyridin-2-yl)pyrrolidin-3-yl)acetonitrile;
(R)-2-(1-(4-(4-((6-((1-acryloylpiperidin-4-yl)amino)-7-methoxyquinazolin-4-yl)amino)-3-fluorophenoxy)pyridin-2-yl)pyrrolidin-3-yl)acetonitrile;
1-(4-((4-((4-((2-(4,6-dihydropyrrolo[3,4-c]pyrazol-5(1H)-yl)pyridin-4-yl)oxy)-2-fluorophenyl)amino)-7-methoxyquinazolin-6-yl)amino)piperidin-1-yl)prop-2-en-1-one;
(3R,4R)-1-(4-(4-((6-((1-acryloylpiperidin-4-yl)amino)-7-methoxyquinazolin-4-yl)amino)-3-fluorophenoxy)pyridin-2-yl)-4-hydroxypyrrolidine-3-carbonitrile;
1-(4-((4-((2-fluoro-4-((2-((3R,4S)-3-fluoro-4-hydroxypyrrolidin-1-yl)pyridin-4-yl)oxy)phenyl)amino)-7-methoxyquinazolin-6-yl)amino)piperidin-1-yl)prop-2-en-1-one;
(R)-1-(4-((4-((2-fluoro-4-(3-(3-fluoropyrrolidin-1-yl)phenoxy)phenyl)amino)-7-methoxyquinazolin-6-yl)amino)piperidin-1-yl)prop-2-en-1-one;
1-(4-((4-((2-fluoro-4-((2-(1-methyl-4,6-dihydropyrrolo[3,4-c]pyrazol-5(1H)-yl)pyridin-4-yl)oxy)phenyl)amino)-7-methoxyquinazolin-6-yl)amino)piperidin-1-yl)prop-2-en-1-one;
1-(4-((4-((2-fluoro-4-((2-((2,2,2-trifluoroethyl)amino)pyridin-4-yl)oxy)phenyl)amino)-7-methoxyquinazolin-6-yl)amino)piperidin-1-yl)prop-2-en-1-one;
4-(4-((6-((1-acryloylpiperidin-4-yl)amino)-7-methoxyquinazolin-4-yl)amino)-3-fluorophenoxy)-N-(2,2,2-trifluoroethyl)picolinamide;
(S)-4-(4-((6-((1-acryloylpiperidin-4-yl)amino)-7-methoxyquinazolin-4-yl)amino)-3-fluorophenoxy)-N-(1,1,1-trifluoropropan-2-yl)picolinamide;
(R)-4-(4-((6-((1-acryloylpiperidin-4-yl)amino)-7-methoxyquinazolin-4-yl)amino)-3-fluorophenoxy)-N-(1,1,1-trifluoropropan-2-yl)picolinamide;
(R)-1-(4-((4-((2-fluoro-4-((2-((1,1,1-trifluoropropan-2-yl)amino)pyridin-4-yl)oxy)phenyl)amino)-7-methoxyquinazolin-6-yl)amino)piperidin-1-yl)prop-2-en-1-one;
(S)-1-(4-((4-((2-fluoro-4-((2-((1,1,1-trifluoropropan-2-yl)amino)pyridin-4-yl)oxy)phenyl)amino)-7-methoxyquinazolin-6-yl)amino)piperidin-1-yl)prop-2-en-1-one;
1-(4-(4-((6-((1-acryloylpiperidin-4-yl)amino)-7-methoxyquinazolin-4-yl)amino)-3-fluorophenoxy)pyridin-2-yl)piperidine-4-carbonitrile;
1-(4-((4-((4-((2-((3,3-difluorocyclobutyl)amino)pyridin-4-yl)oxy)-2-fluorophenyl)amino)-7-methoxyquinazolin-6-yl)amino)piperidin-1-yl)prop-2-en-1-one;
1-(4-((4-((4-((2-(bicyclo[1.1.1]pentan-1-ylamino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((3-fluorocyclobutyl)amino)pyridin-4-yl)oxy)phenyl)amino)-7-methoxyquinazolin-6-yl)amino)piperidin-1-yl)prop-2-en-1-one;
1-(4-((4-((4-((2-((3,3-dimethylcyclobutyl)amino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((3-methoxycyclobutyl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-((6-hydroxyspiro[3.3]heptan-2-yl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-((3-hydroxycyclobutyl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-(oxetan-3-ylamino)pyridin-4-yl)oxy)phenyl)amino)-7-(methoxy-d3)quinazolin-6-yl)amino)piperidin-1-yl)prop-2-en-1-one;
1-(4-((4-((2-fluoro-4-((2-(methyl(oxetan-3-yl)amino)pyridin-4-yl)oxy)phenyl)amino)-7-methoxyquinazolin-6-yl)amino)piperidin-1-yl)prop-2-en-1-one;
1-(4-((4-((2-fluoro-4-((2-(isopropylamino)pyridin-4-yl)oxy)phenyl)amino)-7-methoxyquinazolin-6-yl)amino)piperidin-1-yl)prop-2-en-1-one;
1-(4-((4-((4-((2-((3,3-difluorocyclopentyl)amino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-((1-methylcyclopropyl)amino)pyridin-4-yl)oxy)phenyl)amino)-7-methoxyquinazolin-6-yl)amino)piperidin-1-yl)prop-2-en-1-one;
1-(4-((4-((4-((2-(cyclobutyl(methyl)amino)pyridin-4-yl)oxy)-2-fluorophenyl)amino)-7-methoxyquinazolin-6-yl)amino)piperidin-1-yl)prop-2-en-1-one;
1-(4-((4-((3-chloro-4-((2-(cyclobutylamino)pyridin-4-yl)oxy)phenyl)amino)-7-methoxyquinazolin-6-yl)amino)piperidin-1-yl)prop-2-en-1-one;
1-(4-((4-((4-((2-(cyclobutylamino)pyrimidin-4-yl)oxy)-2-fluorophenyl)amino)-7-methoxyquinazolin-6-yl)amino)piperidin-1-yl)prop-2-en-1-one;
1-(4-((4-((4-((6-(cyclobutylamino)pyridazin-4-yl)oxy)-2-fluorophenyl)amino)-7-methoxyquinazolin-6-yl)amino)piperidin-1-yl)prop-2-en-1-one;
1-(4-((4-((4-((2-((1H-pyrazol-3-yl)amino)pyridin-4-yl)oxy)-2-fluorophenyl)amino)-7-methoxyquinazolin-6-yl)amino)piperidin-1-yl)prop-2-en-1-one;
1-(4-((4-((4-((2-((4H-1,2,4-triazol-3-yl)amino)pyridin-4-yl)oxy)-2-fluorophenyl)amino)-7-methoxyquinazolin-6-yl)amino)piperidin-1-yl)prop-2-en-1-one;
1-(4-((4-((2-fluoro-4-((2-(oxazol-2-ylamino)pyridin-4-yl)oxy)phenyl)amino)-7-methoxyquinazolin-6-yl)amino)piperidin-1-yl)prop-2-en-1-one;
1-(4-((4-((4-((2-((1H-pyrazol-4-yl)amino)pyridin-4-yl)oxy)-2-fluorophenyl)amino)-7-methoxyquinazolin-6-yl)amino)piperidin-1-yl)prop-2-en-1-one;
1-(4-((4-((4-((2-((1,3,4-thiadiazol-2-yl)amino)pyridin-4-yl)oxy)-2-fluorophenyl)amino)-7-methoxyquinazolin-6-yl)amino)piperidin-1-yl)prop-2-en-1-one; and
1-(4-((4-((2-fluoro-4-((2-(thiazol-2-ylamino)pyridin-4-yl)oxy)phenyl)amino)-7-(methoxy-d3)quinazolin-6-yl)amino)piperidin-1-yl)prop-2-en-1-one.

33. A pharmaceutical composition comprising the at least one compound according to claim 1 and at least one additional component selected from pharmaceutically acceptable carriers, pharmaceutically acceptable vehicles, and pharmaceutically acceptable excipients.

34. The pharmaceutical composition according to claim 33, wherein the at least one compound is present in a therapeutically effective amount.

35. A method of treating cancer in a subject in need thereof, comprising administering to the subject an effective amount of the at least one compound according to claim 1 or of the pharmaceutical composition according to claim 33.

36. The method according to claim 35, wherein the cancer is associated with an EGFR or HER2 exon 20 insertion mutation.

37. The method according to claim 35, wherein the cancer is chosen from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, and esophageal cancer.

38. (canceled)

39. (canceled)

40. (canceled)

41. (canceled)

42. (canceled)

43. (canceled)

44. (canceled)

45. (canceled)

46. The method according to claim 35, further comprising administering to the subject in combination with at least one another additional agent.

47. The method according to claim 46, wherein the at least one agent is chosen from gemcitabine, cisplatin, erlotinib, gefitinib, pemetrexed, bevacizumab, cetuximab, trastuzumab, pertuzumab, sorafenib, lapatinib, cobimetinib, selumetinib, and everolimus.

48. The method according to claim 46, wherein the at least one compound of claim 1 and the at least one additional agent are administered concomitantly.

49. The method according to claim 46, wherein the at least one compound of claim 1 and the at least one additional agent are administered sequentially.

50. The method according to claim 46, wherein the pharmaceutical composition of claim 32 and the at least one additional agent are administered concomitantly.

51. The method according to claim 46, wherein the pharmaceutical composition of claim 32 and the at least one additional agent are administered sequentially.
